SEQUENCE LISTING (1460 sequences)

SEQ ID NO: 1 - DNA, 29 bp, Homo sapiens
  modified_base (15)..(15): a, c, g, t, unknown or other
agcaggctgtaggcnatgcgcttctggtg

SEQ ID NO: 2 - DNA, 29 bp, Homo sapiens
  modified_base (15)..(15): a, c, g, t, unknown or other
tggatgattgactcngctgccccgtgcct

SEQ ID NO: 3 - DNA, 29 bp, Homo sapiens
  modified_base (15)..(15): a, c, g, t, unknown or other
caaaagccagaaacncctgacgaatttca

SEQ ID NO: 4 - DNA, 29 bp, Homo sapiens
  modified_base (15)..(15): a, c, g, t, unknown or other
gtgccgggcatacantggtgcccagtaaa

SEQ ID NO: 5 - DNA, 29 bp, Homo sapiens
  modified_base (15)..(15): a, c, g, t, unknown or other
ccgccttcagtgcanctaaaagctaggaa

SEQ ID NO: 6 - DNA, 29 bp, Homo sapiens
  modified_base (15)..(15): a, c, g, t, unknown or other
cttagtttctgagtncagtcttctttgca

SEQ ID NO: 7 - DNA, 29 bp, Homo sapiens
  modified_base (15)..(15): a, c, g, t, unknown or other
gaatgtaggaattcnaaactttgtgcttt

SEQ ID NO: 8 - DNA, 29 bp, Homo sapiens
  modified_base (15)..(15): a, c, g, t, unknown or other
ttctctagatatggnttgctttgagggaa

SEQ ID NO: 9 - DNA, 29 bp, Homo sapiens
  modified_base (15)..(15): a, c, g, t, unknown or other
ttttatagttaaagngatgttcttcaagc

SEQ ID NO: 10 - DNA, 29 bp, Homo sapiens
  modified_base (15)..(15): a, c, g, t, unknown or other
tattttcctaccttntggatcacttcctt

SEQ ID NO: 11 - DNA, 29 bp, Homo sapiens
  modified_base (15)..(15): a, c, g, t, unknown or other
tatcagacgggacantcttagacccttct

SEQ ID NO: 12 - DNA, 29 bp, Homo sapiens
  modified_base (15)..(15): a, c, g, t, unknown or other
cgcctctcctgcacngctgcttgttgagt

SEQ ID NO: 13 - DNA, 29 bp, Homo sapiens
  modified_base (15)..(15): a, c, g, t, unknown or other
gaagggttatgcacnaggacgttagatat

SEQ ID NO: 14 - DNA, 29 bp, Homo sapiens
  modified_base (15)..(15): a, c, g, t, unknown or other
ttccctcaatgatgncgacgtggccccct

SEQ ID NO: 15 - DNA, 29 bp, Homo sapiens
  modified_base (15)..(15): a, c, g, t, unknown or other
tcagccactcaaagncaataataatagta

SEQ ID NO: 16 - DNA, 29 bp, Homo sapiens
  modified_base (15)..(15): a, c, g, t, unknown or other
cgcggcatgtttgcnccgctgacactctg

SEQ ID NO: 17 - DNA, 29 bp, Homo sapiens
  modified_base (15)..(15): a, c, g, t, unknown or other
gaaggatgcccagantgagacaggttgga

SEQ ID NO: 18 - DNA, 29 bp, Homo sapiens
  modified_base (15)..(15): a, c, g, t, unknown or other
gctctcctgagaaanatcgacatgcgaga

SEQ ID NO: 19 - DNA, 29 bp, Homo sapiens
  modified_base (15)..(15): a, c, g, t, unknown or other
ctttcttcatctatncattgggattcttt

SEQ ID NO: 20 - DNA, 29 bp, Homo sapiens
  modified_base (15)..(15): a, c, g, t, unknown or other
tctaagaggatgtgntttgaaaagatgca

SEQ ID NO: 21 - DNA, 29 bp, Homo sapiens
  modified_base (15)..(15): a, c, g, t, unknown or other
ctgactcctcttcangtctgtattataat

SEQ ID NO: 22 - DNA, 29 bp, Homo sapiens
  modified_base (15)..(15): a, c, g, t, unknown or other
tatgtggaacaaccnttgcagatttcaga

SEQ ID NO: 23 - DNA, 29 bp, Homo sapiens
  modified_base (15)..(15): a, c, g, t, unknown or other
ggccagccattgagnttttgaggatcacc

SEQ ID NO: 24 - DNA, 29 bp, Homo sapiens
  modified_base (15)..(15): a, c, g, t, unknown or other
ccgtataatcatctnaattgtctgtaacc

SEQ ID NO: 25 - DNA, 29 bp, Homo sapiens
  modified_base (15)..(15): a, c, g, t, unknown or other
ggggtcattgttctnatctaccatgcacg

SEQ ID NO: 26 - DNA, 29 bp, Homo sapiens
  modified_base (15)..(15): a, c, g, t, unknown or other
cctttcatcttgacntcagtggcttgatg

SEQ ID NO: 27 - DNA, 29 bp, Homo sapiens
  modified_base (15)..(15): a, c, g, t, unknown or other
actacgtgttctgtntgattctcaaagaa

SEQ ID NO: 28 - DNA, 29 bp, Homo sapiens
  modified_base (15)..(15): a, c, g, t, unknown or other
aagatttcaaaatgngctttacccagtat

SEQ ID NO: 29 - DNA, 29 bp, Homo sapiens
  modified_base (15)..(15): a, c, g, t, unknown or other
tcacttggaagttgnataagcatcaccaa

SEQ ID NO: 30 - DNA, 29 bp, Homo sapiens
  modified_base (15)..(15): a, c, g, t, unknown or other
ggattaattggatanattatttggtattt

SEQ ID NO: 31 - DNA, 29 bp, Homo sapiens
  modified_base (15)..(15): a, c, g, t, unknown or other
gataaacggaatgtnattttttacaatct

SEQ ID NO: 32 - DNA, 29 bp, Homo sapiens
  modified_base (15)..(15): a, c, g, t, unknown or other
agagcatttaattcngtgctggaagaggt

SEQ ID NO: 33 - DNA, 29 bp, Homo sapiens
  modified_base (15)..(15): a, c, g, t, unknown or other
gacaatatttagacncattgagtgttaga

SEQ ID NO: 34 - DNA, 29 bp, Homo sapiens
  modified_base (15)..(15): a, c, g, t, unknown or other
tatcattaagactcnatgcatttcctagt

SEQ ID NO: 35 - DNA, 29 bp, Homo sapiens
  modified_base (15)..(15): a, c, g, t, unknown or other
gatagctttcaaatncaatgattagagag

SEQ ID NO: 36 - DNA, 29 bp, Homo sapiens
  modified_base (15)..(15): a, c, g, t, unknown or other
aactaggagtggtcnttcaaaatgatccc

SEQ ID NO: 37 - DNA, 29 bp, Homo sapiens
  modified_base (15)..(15): a, c, g, t, unknown or other
gtggggaaaatatcntttaggattcatca

SEQ ID NO: 38 - DNA, 29 bp, Homo sapiens
  modified_base (15)..(15): a, c, g, t, unknown or other
ccattctgggaaaanaagtaacatgatga

SEQ ID NO: 39 - DNA, 29 bp, Homo sapiens
  modified_base (15)..(15): a, c, g, t, unknown or other
tataactctcctttntgaatgagaagcct

SEQ ID NO: 40 - DNA, 29 bp, Homo sapiens
  modified_base (15)..(15): a, c, g, t, unknown or other
ggttttctgaggcantttgaaggctcatt

SEQ ID NO: 41 - DNA, 29 bp, Homo sapiens
  modified_base (15)..(15): a, c, g, t, unknown or other
tccagaactcttctntagcaaaacaagaa

SEQ ID NO: 42 - DNA, 29 bp, Homo sapiens
  modified_base (15)..(15): a, c, g, t, unknown or other
gtgagaagtagaaancaccactatgatat

SEQ ID NO: 43 - DNA, 29 bp, Homo sapiens
  modified_base (15)..(15): a, c, g, t, unknown or other
tgtagctaagtggtngagctggaattcaa

SEQ ID NO: 44 - DNA, 29 bp, Homo sapiens
  modified_base (15)..(15): a, c, g, t, unknown or other
caatctctgagaaangaatagtaggctct

SEQ ID NO: 45 - DNA, 29 bp, Homo sapiens
  modified_base (15)..(15): a, c, g, t, unknown or other
gaataaagaacaagncaaagcaagcagga

SEQ ID NO: 46 - DNA, 29 bp, Homo sapiens
  modified_base (15)..(15): a, c, g, t, unknown or other
ttgcatcttggcaancctggaatgtcaca

SEQ ID NO: 47 - DNA, 29 bp, Homo sapiens
  modified_base (15)..(15): a, c, g, t, unknown or other
aactatctgaataangcctcttctcaaat

SEQ ID NO: 48 - DNA, 29 bp, Homo sapiens
  modified_base (15)..(15): a, c, g, t, unknown or other
ttattatgcaaccanccttgccttatgtc

SEQ ID NO: 49 - DNA, 29 bp, Homo sapiens
  modified_base (15)..(15): a, c, g, t, unknown or other
cttcaaagattcccnttctattagtcttt

SEQ ID NO: 50 - DNA, 29 bp, Homo sapiens
  modified_base (15)..(15): a, c, g, t, unknown or other
tagacgacactgccngttccagagggcac

SEQ ID NO: 51 - DNA, 29 bp, Homo sapiens
  modified_base (15)..(15): a, c, g, t, unknown or other
ctgaaggcctggatngcattccctttgtc

SEQ ID NO: 52 - DNA, 29 bp, Homo sapiens
  modified_base (15)..(15): a, c, g, t, unknown or other
gataccccacctagntaccactcacactt

SEQ ID NO: 53 - DNA, 29 bp, Homo sapiens
  modified_base (15)..(15): a, c, g, t, unknown or other
atagaagcctaaagnatatccatttctgg

SEQ ID NO: 54 - DNA, 29 bp, Homo sapiens
  modified_base (15)..(15): a, c, g, t, unknown or other
cagactctggagaanttccaatagaagct

SEQ ID NO: 55 - DNA, 29 bp, Homo sapiens
  modified_base (15)..(15): a, c, g, t, unknown or other
gcagcacagtaaacntcaagtttgttaat

SEQ ID NO: 56 - DNA, 29 bp, Homo sapiens
  modified_base (15)..(15): a, c, g, t, unknown or other
aaacaccttcagaanagagcactttaacc

SEQ ID NO: 57 - DNA, 29 bp, Homo sapiens
  modified_base (15)..(15): a, c, g, t, unknown or other
taatacaagtcaccnttctcttcctgttc

SEQ ID NO: 58 - DNA, 29 bp, Homo sapiens
  modified_base (15)..(15): a, c, g, t, unknown or other
tttcttctcagcatnctttatggtctttc

SEQ ID NO: 59 - DNA, 29 bp, Homo sapiens
  modified_base (15)..(15): a, c, g, t, unknown or other
ggagtatcacaagangcaacctggggaac

SEQ ID NO: 60 - DNA, 29 bp, Homo sapiens
  modified_base (15)..(15): a, c, g, t, unknown or other
ccctggaagtcatancattgcagaggctg

SEQ ID NO: 61 - DNA, 29 bp, Homo sapiens
  modified_base (15)..(15): a, c, g, t, unknown or other
cactctgctttgccngctgcctctcaccg

SEQ ID NO: 62 - DNA, 29 bp, Homo sapiens
  modified_base (15)..(15): a, c, g, t, unknown or other
agcaatagacggtgnctcagcctgccgag

SEQ ID NO: 63 - DNA, 29 bp, Homo sapiens
  modified_base (15)..(15): a, c, g, t, unknown or other
accatgcctcgccantggtgaaactgttt

SEQ ID NO: 64 - DNA, 29 bp, Homo sapiens
  modified_base (15)..(15): a, c, g, t, unknown or other
aaagggaataatctntgccagccaccagg

SEQ ID NO: 65 - DNA, 29 bp, Homo sapiens
  modified_base (15)..(15): a, c, g, t, unknown or other
agagggtttattgtngggaagcaagagca

SEQ ID NO: 66 - DNA, 29 bp, Homo sapiens
  modified_base (15)..(15): a, c, g, t, unknown or other
attctttctttcctnaccagctttctctg

SEQ ID NO: 67 - DNA, 29 bp, Homo sapiens
  modified_base (15)..(15): a, c, g, t, unknown or other
ttagtacttgtgggntctttccttttcat

SEQ ID NO: 68 - DNA, 29 bp, Homo sapiens
  modified_base (15)..(15): a, c, g, t, unknown or other
tatcttcttcccaanttcacaatatgagc

SEQ ID NO: 69 - DNA, 29 bp, Homo sapiens
  modified_base (15)..(15): a, c, g, t, unknown or other
cttggtttaagtaangcaccccatacggg

SEQ ID NO: 70 - DNA, 29 bp, Homo sapiens
  modified_base (15)..(15): a, c, g, t, unknown or other
cgacgaaagcccaantgaacgaggtgccc

SEQ ID NO: 71 - DNA, 29 bp, Homo sapiens
  modified_base (15)..(15): a, c, g, t, unknown or other
ggcctaggtgggagngttgcttgaagaaa

SEQ ID NO: 72 - DNA, 29 bp, Homo sapiens
  modified_base (15)..(15): a, c, g, t, unknown or other
aagctcagaggtagnatagagtcggggcg

SEQ ID NO: 73 - DNA, 29 bp, Homo sapiens
  modified_base (15)..(15): a, c, g, t, unknown or other
gatctgttctctaancatctgacctccag

SEQ ID NO: 74 - DNA, 29 bp, Homo sapiens
  modified_base (15)..(15): a, c, g, t, unknown or other
cctgcagactctganatgcctttaccttt

SEQ ID NO: 75 - DNA, 29 bp, Homo sapiens
  modified_base (15)..(15): a, c, g, t, unknown or other
agattcaaggtgtcntaggattccaaagg

SEQ ID NO: 76 - DNA, 29 bp, Homo sapiens
  modified_base (15)..(15): a, c, g, t, unknown or other
caggcaattcctctngtggtggaaagctg

SEQ ID NO: 77 - DNA, 29 bp, Homo sapiens
  modified_base (15)..(15): a, c, g, t, unknown or other
ctcgtccaagcagcntgtccttgtgtgga

SEQ ID NO: 78 - DNA, 29 bp, Homo sapiens
  modified_base (15)..(15): a, c, g, t, unknown or other
aatgcttaaaaatanagtatggcaagttt

SEQ ID NO: 79 - DNA, 29 bp, Homo sapiens
  modified_base (15)..(15): a, c, g, t, unknown or other
gctcttctaaatggnctgggataattcag

SEQ ID NO: 80 - DNA, 29 bp, Homo sapiens
  modified_base (15)..(15): a, c, g, t, unknown or other
ctctttgtttggaanctacactcaggaga

SEQ ID NO: 81 - DNA, 29 bp, Homo sapiens
  modified_base (15)..(15): a, c, g, t, unknown or other
gcattgacttgatgntataaagctccaag

SEQ ID NO: 82 - DNA, 29 bp, Homo sapiens
  modified_base (15)..(15): a, c, g, t, unknown or other
cctgtgtctttcttnaagccatcttaaat

SEQ ID NO: 83 - DNA, 29 bp, Homo sapiens
  modified_base (15)..(15): a, c, g, t, unknown or other
gtaggcccaggtgtnctgaggatgagctg

SEQ ID NO: 84 - DNA, 29 bp, Homo sapiens
  modified_base (15)..(15): a, c, g, t, unknown or other
taacatgcaataacngtcttattacgttt

SEQ ID NO: 85 - DNA, 29 bp, Homo sapiens
  modified_base (15)..(15): a, c, g, t, unknown or other
actatagctcatctntccctgcactctaa

SEQ ID NO: 86 - DNA, 29 bp, Homo sapiens
  modified_base (15)..(15): a, c, g, t, unknown or other
catcctttcacccanaataagtttggaag

SEQ ID NO: 87 - DNA, 29 bp, Homo sapiens
  modified_base (15)..(15): a, c, g, t, unknown or other
gaaagcttgtttggntgtttctcccaaca

SEQ ID NO: 88 - DNA, 29 bp, Homo sapiens
  modified_base (15)..(15): a, c, g, t, unknown or other
ctggagaaacgcctnaggtgaaagcagaa

SEQ ID NO: 89 - DNA, 29 bp, Homo sapiens
  modified_base (15)..(15): a, c, g, t, unknown or other
ttatagtccaaacanctcagtgtgacatt

SEQ ID NO: 90 - DNA, 29 bp, Homo sapiens
  modified_base (15)..(15): a, c, g, t, unknown or other
aaaagaatgactctnctctaatggttatc

SEQ ID NO: 91 - DNA, 29 bp, Homo sapiens
  modified_base (15)..(15): a, c, g, t, unknown or other
tagcatcttttttcntgatgatgttactt

SEQ ID NO: 92 - DNA, 29 bp, Homo sapiens
  modified_base (15)..(15): a, c, g, t, unknown or other
tgaggtttttggcancacttctcccagcc

SEQ ID NO: 93 - DNA, 29 bp, Homo sapiens
  modified_base (15)..(15): a, c, g, t, unknown or other
ggcaaacatagctgnccttgctgctgagc

SEQ ID NO: 94 - DNA, 29 bp, Homo sapiens
  modified_base (15)..(15): a, c, g, t, unknown or other
ggaggctgcctgggnctggctgacaacca

SEQ ID NO: 95 - DNA, 29 bp, Homo sapiens
  modified_base (15)..(15): a, c, g, t, unknown or other
caagagaaattcccngaggaaatgagctg

SEQ ID NO: 96 - DNA, 29 bp, Homo sapiens
  modified_base (15)..(15): a, c, g, t, unknown or other
attgtcttaagagantaataagctttgtg

SEQ ID NO: 97 - DNA, 29 bp, Homo sapiens
  modified_base (15)..(15): a, c, g, t, unknown or other
tttcccaagggcacnttttctggagcaaa

SEQ ID NO: 98 - DNA, 29 bp, Homo sapiens
  modified_base (15)..(15): a, c, g, t, unknown or other
agtgtataagcagcnatgtggacacagca

SEQ ID NO: 99 - DNA, 29 bp, Homo sapiens
  modified_base (15)..(15): a, c, g, t, unknown or other
cctggagtccttgcngtaggatattgact

SEQ ID NO: 100 - DNA, 29 bp, Homo sapiens
  modified_base (15)..(15): a, c, g, t, unknown or other
gtgggggcccatcantgtgggagtttgaa

SEQ ID NO: 101 - DNA, 29 bp, Homo sapiens
  modified_base (15)..(15): a, c, g, t, unknown or other
aaatcaaaactcctnagcacagtgtacat

SEQ ID NO: 102 - DNA, 29 bp, Homo sapiens
  modified_base (15)..(15): a, c, g, t, unknown or other
aatcagtaacattgnaattcaactaagga

SEQ ID NO: 103 - DNA, 29 bp, Homo sapiens
  modified_base (15)..(15): a, c, g, t, unknown or other
ctcctatcctgtcantgtttttgaaattt

SEQ ID NO: 104 - DNA, 29 bp, Homo sapiens
  modified_base (15)..(15): a, c, g, t, unknown or other
tatctaatcaactantaagtgctgccaaa

SEQ ID NO: 105 - DNA, 29 bp, Homo sapiens
  modified_base (15)..(15): a, c, g, t, unknown or other
attgtatggaatcantttctctacaagat

SEQ ID NO: 106 - DNA, 29 bp, Homo sapiens
  modified_base (15)..(15): a, c, g, t, unknown or other
gtcatactatgacangaaacaaagcataa

SEQ ID NO: 107 - DNA, 29 bp, Homo sapiens
  modified_base (15)..(15): a, c, g, t, unknown or other
ctttgtgtggattantacaaaggacaaat

SEQ ID NO: 108 - DNA, 29 bp, Homo sapiens
  modified_base (15)..(15): a, c, g, t, unknown or other
aagtcagtgaacttnaacacaaatcaatc

SEQ ID NO: 109 - DNA, 29 bp, Homo sapiens
  modified_base (15)..(15): a, c, g, t, unknown or other
cctcagcatctctgnagatttccctttat

SEQ ID NO: 110 - DNA, 29 bp, Homo sapiens
  modified_base (15)..(15): a, c, g, t, unknown or other
agcaatttagacctntgtgaatgagaatg

SEQ ID NO: 111 - DNA, 29 bp, Homo sapiens
  modified_base (15)..(15): a, c, g, t, unknown or other
ctctctgagtacctntatgaacaaaattt

SEQ ID NO: 112 - DNA, 29 bp, Homo sapiens
  modified_base (15)..(15): a, c, g, t, unknown or other
aagaaagaaaaccanaggcaaaattgcaa

SEQ ID NO: 113 - DNA, 29 bp, Homo sapiens
  modified_base (15)..(15): a, c, g, t, unknown or other
ggtggaagaggaggngatggataggcctt

SEQ ID NO: 114 - DNA, 29 bp, Homo sapiens
  modified_base (15)..(15): a, c, g, t, unknown or other
acattctataagatnatggttttagcagc

SEQ ID NO: 115 - DNA, 29 bp, Homo sapiens
  modified_base (15)..(15): a, c, g, t, unknown or other
aatggaataatgttnagtaacaggtagtg

SEQ ID NO: 116 - DNA, 29 bp, Homo sapiens
  modified_base (15)..(15): a, c, g, t, unknown or other
ctgctttgatgtccngtgacagacaccct

SEQ ID NO: 117 - DNA, 29 bp, Homo sapiens
  modified_base (15)..(15): a, c, g, t, unknown or other
ccctgaaatccagangcgatcagcctagt

SEQ ID NO: 118 - DNA, 29 bp, Homo sapiens
  modified_base (15)..(15): a, c, g, t, unknown or other
gttaagttccctaanccggtgtcctgacc

SEQ ID NO: 119 - DNA, 29 bp, Homo sapiens
  modified_base (15)..(15): a, c, g, t, unknown or other
ttttgggtattaaanttccatccagttta

SEQ ID NO: 120 - DNA, 29 bp, Homo sapiens
  modified_base (15)..(15): a, c, g, t, unknown or other
ttattgagagaattncagttcaataacac

SEQ ID NO: 121 - DNA, 29 bp, Homo sapiens
  modified_base (15)..(15): a, c, g, t, unknown or other
ttaatttgtacttgnatttttaagatcat

SEQ ID NO: 122 - DNA, 29 bp, Homo sapiens
  modified_base (15)..(15): a, c, g, t, unknown or other
ccaaagttatcatgncaagttagaactaa

SEQ ID NO: 123 - DNA, 29 bp, Homo sapiens
  modified_base (15)..(15): a, c, g, t, unknown or other
ataatagttctcaantccagaagcccact

SEQ ID NO: 124 - DNA, 29 bp, Homo sapiens
  modified_base (15)..(15): a, c, g, t, unknown or other
aatatgaaaaaagtnctaaagctaacatg

SEQ ID NO: 125 - DNA, 29 bp, Homo sapiens
  modified_base (15)..(15): a, c, g, t, unknown or other
aacagctatgctccncttgaaagagcttc

SEQ ID NO: 126 - DNA, 29 bp, Homo sapiens
  modified_base (15)..(15): a, c, g, t, unknown or other
agtttctcgaggacnggaacagtattccc

SEQ ID NO: 127 - DNA, 29 bp, Homo sapiens
  modified_base (15)..(15): a, c, g, t, unknown or other
tatagctgccttcanaaagttcttttaag

SEQ ID NO: 128 - DNA, 29 bp, Homo sapiens
  modified_base (15)..(15): a, c, g, t, unknown or other
gatgaacctacctanagatagagcagagt

SEQ ID NO: 129 - DNA, 29 bp, Homo sapiens
  modified_base (15)..(15): a, c, g, t, unknown or other
cctcacacaagcaangatctcatctccac

SEQ ID NO: 130 - DNA, 29 bp, Homo sapiens
  modified_base (15)..(15): a, c, g, t, unknown or other
aatggtgactgttanccgtctactttggg

SEQ ID NO: 131 - DNA, 29 bp, Homo sapiens
  modified_base (15)..(15): a, c, g, t, unknown or other
tgctgtttctaaacnaaatactaaagctt

SEQ ID NO: 132 - DNA, 29 bp, Homo sapiens
  modified_base (15)..(15): a, c, g, t, unknown or other
catccaaataacacnagtaccttcaacta

SEQ ID NO: 133 - DNA, 29 bp, Homo sapiens
  modified_base (15)..(15): a, c, g, t, unknown or other
tcccggaagtggacnttagcagcgcctgt

SEQ ID NO: 134 - DNA, 29 bp, Homo sapiens
  modified_base (15)..(15): a, c, g, t, unknown or other
gtttgctactccgtnatttacttgcatct

SEQ ID NO: 135 - DNA, 29 bp, Homo sapiens
  modified_base (15)..(15): a, c, g, t, unknown or other
tcgtgcattcaactngttcccgctcatgg

SEQ ID NO: 136 - DNA, 29 bp, Homo sapiens
  modified_base (15)..(15): a, c, g, t, unknown or other
aaggctttgcaaganaaaacatccacttt

SEQ ID NO: 137 - DNA, 29 bp, Homo sapiens
  modified_base (15)..(15): a, c, g, t, unknown or other
atctactaagatgtnagacttttttcaca

SEQ ID NO: 138 - DNA, 29 bp, Homo sapiens
  modified_base (15)..(15): a, c, g, t, unknown or other
tattttattacatcntgctcagtctgtac

SEQ ID NO: 139 - DNA, 29 bp, Homo sapiens
  modified_base (15)..(15): a, c, g, t, unknown or other
gaatatataggttcntagaagttggtatc

SEQ ID NO: 140 - DNA, 29 bp, Homo sapiens
  modified_base (15)..(15): a, c, g, t, unknown or other
acagaagaagtcacngggactgacaatct

SEQ ID NO: 141 - DNA, 29 bp, Homo sapiens
  modified_base (15)..(15): a, c, g, t, unknown or other
aacttaaaattgccntgcttaaagacctg

SEQ ID NO: 142 - DNA, 29 bp, Homo sapiens
  modified_base (15)..(15): a, c, g, t, unknown or other
aaatttgtactaganttctttataacaca

SEQ ID NO: 143 - DNA, 29 bp, Homo sapiens
  modified_base (15)..(15): a, c, g, t, unknown or other
ttcaccgcgatcccnaaatgcttacatgt

SEQ ID NO: 144 - DNA, 29 bp, Homo sapiens
  modified_base (15)..(15): a, c, g, t, unknown or other
gatgctattcttagncttcacgttaattg

SEQ ID NO: 145 - DNA, 29 bp, Homo sapiens
  modified_base (15)..(15): a, c, g, t, unknown or other
aggcctaccaatttntcagaatagagaaa

SEQ ID NO: 146 - DNA, 29 bp, Homo sapiens
  modified_base (15)..(15): a, c, g, t, unknown or other
ggagtgactcatctnattagagaatttgg

SEQ ID NO: 147 - DNA, 29 bp, Homo sapiens
  modified_base (15)..(15): a, c, g, t, unknown or other
aagagtcaacagttnggatgcattacgtt

SEQ ID NO: 148 - DNA, 29 bp, Homo sapiens
  modified_base (15)..(15): a, c, g, t, unknown or other
aagattagttcacangtatgatgcaaact

SEQ ID NO: 149 - DNA, 29 bp, Homo sapiens
  modified_base (15)..(15): a, c, g, t, unknown or other
atttaggacagagangtttcacagacaac

SEQ ID NO: 150 - DNA, 29 bp, Homo sapiens
  modified_base (15)..(15): a, c, g, t, unknown or other
ttcctcaaggctgtnttaaggttacattt

SEQ ID NO: 151 - DNA, 29 bp, Homo sapiens
  modified_base (15)..(15): a, c, g, t, unknown or other
attagtattgaaacngcagaattaggtta

SEQ ID NO: 152 - DNA, 29 bp, Homo sapiens
  modified_base (15)..(15): a, c, g, t, unknown or other
ggctttctatgctgnacttagcagctacg

SEQ ID NO: 153 - DNA, 29 bp, Homo sapiens
  modified_base (15)..(15): a, c, g, t, unknown or other
ttttctgaaactaangatggaataaccat

SEQ ID NO: 154 - DNA, 29 bp, Homo sapiens
  modified_base (15)..(15): a, c, g, t, unknown or other
aaaatccctgagatnatttctatgcgatg

SEQ ID NO: 155 - DNA, 29 bp, Homo sapiens
  modified_base (15)..(15): a, c, g, t, unknown or other
gcaatctgttttatntcaggtggattaca

SEQ ID NO: 156 - DNA, 29 bp, Homo sapiens
  modified_base (15)..(15): a, c, g, t, unknown or other
agctaagaagtagcngagctgagaaacct

SEQ ID NO: 157 - DNA, 29 bp, Homo sapiens
  modified_base (15)..(15): a, c, g, t, unknown or other
caagggtgccagccnccaccagaagccag

SEQ ID NO: 158 - DNA, 29 bp, Homo sapiens
  modified_base (15)..(15): a, c, g, t, unknown or other
ttcttcctattggcntgaggataacagag

SEQ ID NO: 159 - DNA, 29 bp, Homo sapiens
  modified_base (15)..(15): a, c, g, t, unknown or other
caataaaaccataangattggtgttattt

SEQ ID NO: 160 - DNA, 29 bp, Homo sapiens
  modified_base (15)..(15): a, c, g, t, unknown or other
catctctcgctatcntcttctgcctctca

SEQ ID NO: 161 - DNA, 29 bp, Homo sapiens
  modified_base (15)..(15): a, c, g, t, unknown or other
ttgcatcggccctcngataaagagaagca

SEQ ID NO: 162 - DNA, 29 bp, Homo sapiens
  modified_base (15)..(15): a, c, g, t, unknown or other
cagtacggctttcantaatggtgatgaac

SEQ ID NO: 163 - DNA, 29 bp, Homo sapiens
  modified_base (15)..(15): a, c, g, t, unknown or other
aaataagatgtatanggtaaaccttagag

SEQ ID NO: 164 - DNA, 29 bp, Homo sapiens
  modified_base (15)..(15): a, c, g, t, unknown or other
acatatcccaggagntaagatgaagctaa

SEQ ID NO: 165 - DNA, 29 bp, Homo sapiens
  modified_base (15)..(15): a, c, g, t, unknown or other
aattcaaaaactgtnccatattaagctgt

SEQ ID NO: 166 - DNA, 29 bp, Homo sapiens
  modified_base (15)..(15): a, c, g, t, unknown or other
tttgtgtttaaaatngcccaagctccact

SEQ ID NO: 167 - DNA, 29 bp, Homo sapiens
  modified_base (15)..(15): a, c, g, t, unknown or other
tcagtaattttctanaaagtcagatggcc

SEQ ID NO: 168 - DNA, 29 bp, Homo sapiens
  modified_base (15)..(15): a, c, g, t, unknown or other
tacccattgggaatncactttctgcattt

SEQ ID NO: 169 - DNA, 29 bp, Homo sapiens
  modified_base (15)..(15): a, c, g, t, unknown or other
ggacttcgtagggangtgtcagttaagac

SEQ ID NO: 170 - DNA, 29 bp, Homo sapiens
  modified_base (15)..(15): a, c, g, t, unknown or other
cagcagaaacctcgnaggcaattccagag

SEQ ID NO: 171 - DNA, 29 bp, Homo sapiens
  modified_base (15)..(15): a, c, g, t, unknown or other
atcttcctgggcttntcaactggatacct

SEQ ID NO: 172 - DNA, 29 bp, Homo sapiens
  modified_base (15)..(15): a, c, g, t, unknown or other
gttctcctccagtgngttctgggtaccag

SEQ ID NO: 173 - DNA, 29 bp, Homo sapiens
  modified_base (15)..(15): a, c, g, t, unknown or other
agcacctgacccacngcaatgggaataaa

SEQ ID NO: 174 - DNA, 29 bp, Homo sapiens
  modified_base (15)..(15): a, c, g, t, unknown or other
tctcagagcttgggntatgagaaatcagg

SEQ ID NO: 175 - DNA, 29 bp, Homo sapiens
  modified_base (15)..(15): a, c, g, t, unknown or other
cagtgctgggtgaantgactggcatctcc

SEQ ID NO: 176 - DNA, 29 bp, Homo sapiens
  modified_base (15)..(15): a, c, g, t, unknown or other
tgctgtgatgcaatntaggctatgagtgg

SEQ ID NO: 177 - DNA, 29 bp, Homo sapiens
  modified_base (15)..(15): a, c, g, t, unknown or other
atgcagtggtaacanaggaacatttaaca

SEQ ID NO: 178 - DNA, 29 bp, Homo sapiens
  modified_base (15)..(15): a, c, g, t, unknown or other
gtgaagaattgatanacgtaaagggttaa

SEQ ID NO: 179 - DNA, 29 bp, Homo sapiens
  modified_base (15)..(15): a, c, g, t, unknown or other
cagaaatctaagcancaatccctccaacg

SEQ ID NO: 180 - DNA, 29 bp, Homo sapiens
  modified_base (15)..(15): a, c, g, t, unknown or other
actaaaacttcaggnaaaatcgtgtgttc

SEQ ID NO: 181 - DNA, 29 bp, Homo sapiens
  modified_base (15)..(15): a, c, g, t, unknown or other
agtcggtcagctatntggggcctgaatta

SEQ ID NO: 182 - DNA, 29 bp, Homo sapiens
  modified_base (15)..(15): a, c, g, t, unknown or other
tccagaagaccatgnaacttgtctatagt

SEQ ID NO: 183 - DNA, 29 bp, Homo sapiens
  modified_base (15)..(15): a, c, g, t, unknown or other
aaccatcaaacaccnggaacccatcttgg

SEQ ID NO: 184 - DNA, 29 bp, Homo sapiens
  modified_base (15)..(15): a, c, g, t, unknown or other
cagtgcttcccagtnactgaacaaaaact

SEQ ID NO: 185 - DNA, 29 bp, Homo sapiens
  modified_base (15)..(15): a, c, g, t, unknown or other
tatttttataggccnacaccgaactgtgt

SEQ ID NO: 186 - DNA, 29 bp, Homo sapiens
  modified_base (15)..(15): a, c, g, t, unknown or other
atgcaccccaaaacntaatatctatgtgt

SEQ ID NO: 187 - DNA, 29 bp, Homo sapiens
  modified_base (15)..(15): a, c, g, t, unknown or other
ggagagcagaactcnctgcaagcaaaaag

SEQ ID NO: 188 - DNA, 29 bp, Homo sapiens
  modified_base (15)..(15): a, c, g, t, unknown or other
tccctcctatttgcnatcaaccctggaac

SEQ ID NO: 189 - DNA, 29 bp, Homo sapiens
  modified_base (15)..(15): a, c, g, t, unknown or other
aattggagaatgaanaaaatccagaaaat

SEQ ID NO: 190 - DNA, 29 bp, Homo sapiens
  modified_base (15)..(15): a, c, g, t, unknown or other
tgttctgtggcggcngagccatgactcag

SEQ ID NO: 191 - DNA, 29 bp, Homo sapiens
  modified_base (15)..(15): a, c, g, t, unknown or other
cggacggctccaacnactcagtctcctgt

SEQ ID NO: 192 - DNA, 29 bp, Homo sapiens
  modified_base (15)..(15): a, c, g, t, unknown or other
cagccaggatcaggnatactgggagttca

SEQ ID NO: 193 - DNA, 29 bp, Homo sapiens
  modified_base (15)..(15): a, c, g, t, unknown or other
ccagggtccacatgntctttttctaactt

SEQ ID NO: 194 - DNA, 29 bp, Homo sapiens
  modified_base (15)..(15): a, c, g, t, unknown or other
tgccagagggctttnaggacagaaatggg

SEQ ID NO: 195 - DNA, 29 bp, Homo sapiens
  modified_base (15)..(15): a, c, g, t, unknown or other
ggaaagagcggtaangatggttcaggaat

SEQ ID NO: 196 - DNA, 29 bp, Homo sapiens
  modified_base (15)..(15): a, c, g, t, unknown or other
ctggttgctattttntactcccggagtca

SEQ ID NO: 197 - DNA, 29 bp, Homo sapiens
  modified_base (15)..(15): a, c, g, t, unknown or other
taatgcataccaggngcaaagatcagccc

SEQ ID NO: 198 - DNA, 29 bp, Homo sapiens
  modified_base (15)..(15): a, c, g, t, unknown or other
gccaatgacttcccngttgtaaccctcac

SEQ ID NO: 199 - DNA, 29 bp, Homo sapiens
  modified_base (15)..(15): a, c, g, t, unknown or other
ctttgattttagtanaagtggactctgcc

SEQ ID NO: 200 - DNA, 29 bp, Homo sapiens
  modified_base (15)..(15): a, c, g, t, unknown or other
gagttgggggtgttnttgcacctggctgg

SEQ ID NO: 201 - DNA, 29 bp, Homo sapiens
  modified_base (15)..(15): a, c, g, t, unknown or other
tttatgcacttaaanagggaacagagatg

SEQ ID NO: 202 - DNA, 29 bp, Homo sapiens
  modified_base (15)..(15): a, c, g, t, unknown or other
tccctgtggtatctntcatgtacttggac

SEQ ID NO: 203 - DNA, 29 bp, Homo sapiens
  modified_base (15)..(15): a, c, g, t, unknown or other
accttcatattcaanggtggatgtcctgg

SEQ ID NO: 204 - DNA, 29 bp, Homo sapiens
  modified_base (15)..(15): a, c, g, t, unknown or other
tcttgatggaggaantcaagggaggcttc

SEQ ID NO: 205 - DNA, 29 bp, Homo sapiens
  modified_base (15)..(15): a, c, g, t, unknown or other
atcctgaggatcggnctccccctggggtc

SEQ ID NO: 206 - DNA, 29 bp, Homo sapiens
  modified_base (15)..(15): a, c, g, t, unknown or other
tgttggaatgtccangaacagaaatagta

SEQ ID NO: 207 - DNA, 29 bp, Homo sapiens
  modified_base (15)..(15): a, c, g, t, unknown or other
tattttcacagtacnatgttcagattgag

SEQ ID NO: 208 - DNA, 29 bp, Homo sapiens
  modified_base (15)..(15): a, c, g, t, unknown or other
gtattagtagaagangtttgtaaatgtga

SEQ ID NO: 209 - DNA, 29 bp, Homo sapiens
  modified_base (15)..(15): a, c, g, t, unknown or other
gcttctgggacacanagatgctgaacagg

SEQ ID NO: 210 - DNA, 29 bp, Homo sapiens
  modified_base (15)..(15): a, c, g, t, unknown or other
tctgatcacaattanaggcctatcctttc

SEQ ID NO: 211 - DNA, 29 bp, Homo sapiens
  modified_base (15)..(15): a, c, g, t, unknown or other
gaagacacaggttcnttatctctctttaa

SEQ ID NO: 212 - DNA, 29 bp, Homo sapiens
  modified_base (15)..(15): a, c, g, t, unknown or other
gaatcggttagattngtattcaggtaaca

SEQ ID NO: 213 - DNA, 29 bp, Homo sapiens
  modified_base (15)..(15): a, c, g, t, unknown or other
ctctgcgtaatctgnttttttcccagcag

SEQ ID NO: 214 - DNA, 29 bp, Homo sapiens
  modified_base (15)..(15): a, c, g, t, unknown or other
atgaccaaaggcacnagacataactactg

SEQ ID NO: 215 - DNA, 29 bp, Homo sapiens
  modified_base (15)..(15): a, c, g, t, unknown or other
agaggaagcatttcnataactcataggtt

SEQ ID NO: 216 - DNA, 29 bp, Homo sapiens
  modified_base (15)..(15): a, c, g, t, unknown or other
atgaaactgtaattntctagttcggagtt

SEQ ID NO: 217 - DNA, 29 bp, Homo sapiens
  modified_base (15)..(15): a, c, g, t, unknown or other
cacagcagtttctcntttcatagccgatt

SEQ ID NO: 218 - DNA, 29 bp, Homo sapiens
  modified_base (15)..(15): a, c, g, t, unknown or other
gagtaagtcactgtngaacctttatctga

SEQ ID NO: 219 - DNA, 29 bp, Homo sapiens
  modified_base (15)..(15): a, c, g, t, unknown or other
aaatcaggatacctntttcactataaaca

SEQ ID NO: 220 - DNA, 29 bp, Homo sapiens
  modified_base (15)..(15): a, c, g, t, unknown or other
taaacaaactcttcnagcaaataacaggc

SEQ ID NO: 221 - DNA, 29 bp, Homo sapiens
  modified_base (15)..(15): a, c, g, t, unknown or other
gaaaggatgccagcnttgttgtgtttgga

SEQ ID NO: 222 - DNA, 29 bp, Homo sapiens
  modified_base (15)..(15): a, c, g, t, unknown or other
ccaatatgttctctnaaagactcaaagat

SEQ ID NO: 223 - DNA, 29 bp, Homo sapiens
  modified_base (15)..(15): a, c, g, t, unknown or other
ccataactgtacacnggaagagttatgca

SEQ ID NO: 224 - DNA, 29 bp, Homo sapiens
  modified_base (15)..(15): a, c, g, t, unknown or other
aataataatcatggngagcaacagataaa

SEQ ID NO: 225 - DNA, 29 bp, Homo sapiens
  modified_base (15)..(15): a, c, g, t, unknown or other
aaactgttcattcanagcaactctcagta

SEQ ID NO: 226 - DNA, 29 bp, Homo sapiens
  modified_base (15)..(15): a, c, g, t, unknown or other
cagtttacgtaatcnggaaaataagagtg

SEQ ID NO: 227 - DNA, 29 bp, Homo sapiens
  modified_base (15)..(15): a, c, g, t, unknown or other
ctttctagtctattnaggcatttttgttg

SEQ ID NO: 228 - DNA, 29 bp, Homo sapiens
  modified_base (15)..(15): a, c, g, t, unknown or other
acaactgtatttgcntaagcaccccataa

SEQ ID NO: 229 - DNA, 29 bp, Homo sapiens
  modified_base (15)..(15): a, c, g, t, unknown or other
aattgtacaggcccntgaaagtggatgat

SEQ ID NO: 230 - DNA, 29 bp, Homo sapiens
  modified_base (15)..(15): a, c, g, t, unknown or other
aatttttgtctttancataaccctgctgt

SEQ ID NO: 231 - DNA, 29 bp, Homo sapiens
  modified_base (15)..(15): a, c, g, t, unknown or other
gctggggtggcaacntaatccaaaattcc

SEQ ID NO: 232 - DNA, 29 bp, Homo sapiens
  modified_base (15)..(15): a, c, g, t, unknown or other
tacttgtatgcctancacgatgtaatttt

SEQ ID NO: 233 - DNA, 29 bp, Homo sapiens
  modified_base (15)..(15): a, c, g, t, unknown or other
gtaaaacttatcctngggaagtgcattgc

SEQ ID NO: 234 - DNA, 29 bp, Homo sapiens
  modified_base (15)..(15): a, c, g, t, unknown or other
agtttctatatgcanatcatgactgaata

SEQ ID NO: 235 - DNA, 29 bp, Homo sapiens
  modified_base (15)..(15): a, c, g, t, unknown or other
tggatatcttggctntgtcactgtttccc

SEQ ID NO: 236 - DNA, 29 bp, Homo sapiens
  modified_base (15)..(15): a, c, g, t, unknown or other
gtgctgggcaatcanataggcactgtgaa

SEQ ID NO: 237 - DNA, 29 bp, Homo sapiens
  modified_base (15)..(15): a, c, g, t, unknown or other
cctgtaaaaagatancattcccttccaat

SEQ ID NO: 238 - DNA, 29 bp, Homo sapiens
  modified_base (15)..(15): a, c, g, t, unknown or other
aataggataaatacngataatcttaataa

SEQ ID NO: 239 - DNA, 29 bp, Homo sapiens
  modified_base (15)..(15): a, c, g, t, unknown or other
atggcttaaattatnctgttaaaagcaat

SEQ ID NO: 240 - DNA, 29 bp, Homo sapiens
  modified_base (15)..(15): a, c, g, t, unknown or other
taccatgctattcanttcggtatatgcca

SEQ ID NO: 241 - DNA, 29 bp, Homo sapiens
  modified_base (15)..(15): a, c, g, t, unknown or other
caaataacataatgnttgcatgcacatag

SEQ ID NO: 242 - DNA, 29 bp, Homo sapiens
  modified_base (15)..(15): a, c, g, t, unknown or other
agttattttggtggnaacggcttaggtgt

SEQ ID NO: 243 - DNA, 29 bp, Homo sapiens
  modified_base (15)..(15): a, c, g, t, unknown or other
caagtacaaggtctntgcagtgtgttggc

SEQ ID NO: 244 - DNA, 29 bp, Homo sapiens
  modified_base (15)..(15): a, c, g, t, unknown or other
tagagtacctgaagntgtacctcaaaata

SEQ ID NO: 245 - DNA, 29 bp, Homo sapiens
  modified_base (15)..(15): a, c, g, t, unknown or other
gttaccagtccttanggcatttgggtaag

SEQ ID NO: 246 - DNA, 29 bp, Homo sapiens
  modified_base (15)..(15): a, c, g, t, unknown or other
ctgtcctttcatagngctcagtactagaa

SEQ ID NO: 247 - DNA, 29 bp, Homo sapiens
  modified_base (15)..(15): a, c, g, t, unknown or other
tactcaatgtcagtntatccatcctcaat

SEQ ID NO: 248 - DNA, 29 bp, Homo sapiens
  modified_base (15)..(15): a, c, g, t, unknown or other
catggaagtccatanataaattttgtgta

SEQ ID NO: 249 - DNA, 29 bp, Homo sapiens
  modified_base (15)..(15): a, c, g, t, unknown or other
atttcagaatcctangatttttcctgcca

SEQ ID NO: 250 - DNA, 29 bp, Homo sapiens
  modified_base (15)..(15): a, c, g, t, unknown or other
cttagttttcaattnttaaaatgtcaagc

SEQ ID NO: 251 - DNA, 29 bp, Homo sapiens
  modified_base (15)..(15): a, c, g, t, unknown or other
gtgtctaaccataanggaagtaaagagaa

SEQ ID NO: 252 - DNA, 29 bp, Homo sapiens
  modified_base (15)..(15): a, c, g, t, unknown or other
atccaccatcttctntattacaacaacat

SEQ ID NO: 253 - DNA, 29 bp, Homo sapiens
  modified_base (15)..(15): a, c, g, t, unknown or other
ctctattaagttacncacatttccaaata

SEQ ID NO: 254 - DNA, 29 bp, Homo sapiens
  modified_base (15)..(15): a, c, g, t, unknown or other
taatctctgggtacnaagactagtaactg

SEQ ID NO: 255 - DNA, 29 bp, Homo sapiens
  modified_base (15)..(15): a, c, g, t, unknown or other
gaaatgaaaattacnaagaaagaactcat

SEQ ID NO: 256 - DNA, 29 bp, Homo sapiens
  modified_base (15)..(15): a, c, g, t, unknown or other
gtgccggtgagtctntgcgcttatcagga

SEQ ID NO: 257 - DNA, 29 bp, Homo sapiens
  modified_base (15)..(15): a, c, g, t, unknown or other
aaatctgttaagccnaaatggtttttttt

SEQ ID NO: 258 - DNA, 29 bp, Homo sapiens
  modified_base (15)..(15): a, c, g, t, unknown or other
gaaagtatgtctacnttgaggctgtattt

SEQ ID NO: 259 - DNA, 29 bp, Homo sapiens
  modified_base (15)..(15): a, c, g, t, unknown or other
aacttgatactttcngaaatctaaccaag

SEQ ID NO: 260 - DNA, 29 bp, Homo sapiens
  modified_base (15)..(15): a, c, g, t, unknown or other
tacatagaaagtacnccaagatacatcga

SEQ ID NO: 261 - DNA, 29 bp, Homo sapiens
  modified_base (15)..(15): a, c, g, t, unknown or other
agcatctagaatgtnaaaaaaatgcaact

SEQ ID NO: 262 - DNA, 29 bp, Homo sapiens
  modified_base (15)..(15): a, c, g, t, unknown or other
tctttgcattaatcnatgaatcttaccat

SEQ ID NO: 263 - DNA, 29 bp, Homo sapiens
  modified_base (15)..(15): a, c, g, t, unknown or other
aattgtcaagtagantttggtttaagtta

SEQ ID NO: 264 - DNA, 29 bp, Homo sapiens
  modified_base (15)..(15): a, c, g, t, unknown or other
agcatggtttaaagnctgattttactcta

SEQ ID NO: 265 - DNA, 29 bp, Homo sapiens
  modified_base (15)..(15): a, c, g, t, unknown or other
ttagctgtattttgntgatgtgatcaata

SEQ ID NO: 266 - DNA, 29 bp, Homo sapiens
  modified_base (15)..(15): a, c, g, t, unknown or other
ttgatttcctattangtattcatcttaga

SEQ ID NO: 267 - DNA, 29 bp, Homo sapiens
  modified_base (15)..(15): a, c, g, t, unknown or other
acatttccatgaganacatatttttagaa

SEQ ID NO: 268 - DNA, 29 bp, Homo sapiens
  modified_base (15)..(15): a, c, g, t, unknown or other
cgactgtcttttttngtgaataacagtga

SEQ ID NO: 269 - DNA, 29 bp, Homo sapiens
  modified_base (15)..(15): a, c, g, t, unknown or other
agcatgagttctccngtaggtactaattg

SEQ ID NO: 270 - DNA, 29 bp, Homo sapiens
  modified_base (15)..(15): a, c, g, t, unknown or other
gttaatttctccggnggtcactctatccg

SEQ ID NO: 271 - DNA, 29 bp, Homo sapiens
  modified_base (15)..(15): a, c, g, t, unknown or other
tgtcagattccaccnttgatgttctttct

SEQ ID NO: 272 - DNA, 29 bp, Homo sapiens
  modified_base (15)..(15): a, c, g, t, unknown or other
ggaagtaaatatgtnactgaatctatcat

SEQ ID NO: 273 - DNA, 29 bp, Homo sapiens
  modified_base (15)..(15): a, c, g, t, unknown or other
aaattcttcaccagnaaagggattaacat

SEQ ID NO: 274 - DNA, 29 bp, Homo sapiens
  modified_base (15)..(15): a, c, g, t, unknown or other
gcaatgtaaagagcntcgtactaagaaaa

SEQ ID NO: 275 - DNA, 29 bp, Homo sapiens
  modified_base (15)..(15): a, c, g, t, unknown or other
tttataaaccgaccntatctttcgatatc

SEQ ID NO: 276 - DNA, 29 bp, Homo sapiens
  modified_base (15)..(15): a, c, g, t, unknown or other
cattcatttattcantctcaaaatgttta

SEQ ID NO: 277 - DNA, 29 bp, Homo sapiens
  modified_base (15)..(15): a, c, g, t, unknown or other
atgtaattttctcancctgatgaggaaag

SEQ ID NO: 278 - DNA, 29 bp, Homo sapiens
  modified_base (15)..(15): a, c, g, t, unknown or other
gacctgatagaagtnagagataaccctgc

SEQ ID NO: 279 - DNA, 29 bp, Homo sapiens
  modified_base (15)..(15): a, c, g, t, unknown or other
ccgcattttcacttngtaattcatcagag

SEQ ID NO: 280 - DNA, 29 bp, Homo sapiens
  modified_base (15)..(15): a, c, g, t, unknown or other
agaaaatggaaagcnattagagtaaaaga

SEQ ID NO: 281 - DNA, 29 bp, Homo sapiens
  modified_base (15)..(15): a, c, g, t, unknown or other
gtgaagacggggccnggcttatgatagaa

SEQ ID NO: 282 - DNA, 29 bp, Homo sapiens
  modified_base (15)..(15): a, c, g, t, unknown or other
taaataatgcatgtnctaaattcataagt

SEQ ID NO: 283 - DNA, 29 bp, Homo sapiens
  modified_base (15)..(15): a, c, g, t, unknown or other
ttacagaccaaaatnaaataacataattc

SEQ ID NO: 284 - DNA, 29 bp, Homo sapiens
  modified_base (15)..(15): a, c, g, t, unknown or other
tgagaaagtgatgcntaagcagagaaatg

SEQ ID NO: 285 - DNA, 29 bp, Homo sapiens
  modified_base (15)..(15): a, c, g, t, unknown or other
agatgtataacatgntttaaaaataaaca

SEQ ID NO: 286 - DNA, 29 bp, Homo sapiens
  modified_base (15)..(15): a, c, g, t, unknown or other
caggcaaagcaaatnttggtgaattcttt

SEQ ID NO: 287 - DNA, 29 bp, Homo sapiens
  modified_base (15)..(15): a, c, g, t, unknown or other
cattttgcagatacngaaagtaaggcaca

SEQ ID NO: 288 - DNA, 29 bp, Homo sapiens
  modified_base (15)..(15): a, c, g, t, unknown or other
attttcaaataactngttgtctaattcag

SEQ ID NO: 289 - DNA, 29 bp, Homo sapiens
  modified_base (15)..(15): a, c, g, t, unknown or other
ttaattgtagagcangaactatgtgacta

SEQ ID NO: 290 - DNA, 29 bp, Homo sapiens
  modified_base (15)..(15): a, c, g, t, unknown or other
aataataattaatangctaaaaactcaag

SEQ ID NO: 291 - DNA, 29 bp, Homo sapiens
  modified_base (15)..(15): a, c, g, t, unknown or other
ttatatacaaactgngatttctttccccc

SEQ ID NO: 292 - DNA, 29 bp, Homo sapiens
  modified_base (15)..(15): a, c, g, t, unknown or other
ccaaaagtccattgncatttcaagcatag

SEQ ID NO: 293 - DNA, 29 bp, Homo sapiens
  modified_base (15)..(15): a, c, g, t, unknown or other
ttagtagagcagagntttatccagactga

SEQ ID NO: 294 - DNA, 29 bp, Homo sapiens
  modified_base (15)..(15): a, c, g, t, unknown or other
ccctatattccaatngtactggaacccag

SEQ ID NO: 295 - DNA, 29 bp, Homo sapiens
  modified_base (15)..(15): a, c, g, t, unknown or other
tcaaatacgaaaagncatacatacattta

SEQ ID NO: 296 - DNA, 29 bp, Homo sapiens
  modified_base (15)..(15): a, c, g, t, unknown or other
tttcttagtatagcntcgtaataaatctt

SEQ ID NO: 297 - DNA, 29 bp, Homo sapiens
  modified_base (15)..(15): a, c, g, t, unknown or other
ccagttgaagacatnagtgcccagttgcc

SEQ ID NO: 298 - DNA, 29 bp, Homo sapiens
  modified_base (15)..(15): a, c, g, t, unknown or other
catttatttagagangcatcaagaaaaat

SEQ ID NO: 299 - DNA, 29 bp, Homo sapiens
  modified_base (15)..(15): a, c, g, t, unknown or other
atcagagcacttggnttgtgacaagggat

SEQ ID NO: 300 - DNA, 29 bp, Homo sapiens
  modified_base (15)..(15): a, c, g, t, unknown or other
ttatcctgattttanggatgggagaattt

SEQ ID NO: 301 - DNA, 29 bp, Homo sapiens
  modified_base (15)..(15): a, c, g, t, unknown or other
gtgacagatagctcngtttcttccctgcc

SEQ ID NO: 302 - DNA, 29 bp, Homo sapiens
  modified_base (15)..(15): a, c, g, t, unknown or other
caagaatgagaatangggaaatttttaag

SEQ ID NO: 303 - DNA, 29 bp, Homo sapiens
  modified_base (15)..(15): a, c, g, t, unknown or other
tcatcgtcattgtcntcatcatcatcatc

SEQ ID NO: 304 - DNA, 29 bp, Homo sapiens
  modified_base (15)..(15): a, c, g, t, unknown or other
attgcagattacaanacaaaaatcttcaa

SEQ ID NO: 305 - DNA, 29 bp, Homo sapiens
  modified_base (15)..(15): a, c, g, t, unknown or other
ggacacacacttacntttccaaacatatt

SEQ ID NO: 306 - DNA, 29 bp, Homo sapiens
  modified_base (15)..(15): a, c, g, t, unknown or other
aacagcaatggatanctgctgagtataga

SEQ ID NO: 307 - DNA, 29 bp, Homo sapiens
  modified_base (15)..(15): a, c, g, t, unknown or other
ttttttatgggaagnctacatcttacgaa

SEQ ID NO: 308 - DNA, 29 bp, Homo sapiens
  modified_base (15)..(15): a, c, g, t, unknown or other
aggaattaaaccagntgtaacggctataa

SEQ ID NO: 309 - DNA, 29 bp, Homo sapiens
  modified_base (15)..(15): a, c, g, t, unknown or other
caaaagttgtccagnatgttgtcgcttgc

SEQ ID NO: 310 - DNA, 29 bp, Homo sapiens
  modified_base (15)..(15): a, c, g, t, unknown or other
atagccattgatcanaaactgactatcat

SEQ ID NO: 311 - DNA, 29 bp, Homo sapiens
  modified_base (15)..(15): a, c, g, t, unknown or other
ccctgggtttaatgncaaatcaaggcaaa

SEQ ID NO: 312 - DNA, 29 bp, Homo sapiens
  modified_base (15)..(15): a, c, g, t, unknown or other
gataaagcagtcttntccaatggaaaaat

SEQ ID NO: 313 - DNA, 29 bp, Homo sapiens
  modified_base (15)..(15): a, c, g, t, unknown or other
gctaccattctcacntaatctagccaagt

SEQ ID NO: 314 - DNA, 29 bp, Homo sapiens
  modified_base (15)..(15): a, c, g, t, unknown or other
acactggacaagcanaaactggcacataa

SEQ ID NO: 315 - DNA, 29 bp, Homo sapiens
  modified_base (15)..(15): a, c, g, t, unknown or other
atgttcaagcatagncttggctactgagt

SEQ ID NO: 316 - DNA, 29 bp, Homo sapiens
  modified_base (15)..(15): a, c, g, t, unknown or other
agatcaatattcagnaacaatcagttccc

SEQ ID NO: 317 - DNA, 29 bp, Homo sapiens
  modified_base (15)..(15): a, c, g, t, unknown or other
agtggccggggaagntgttttggaagcag

SEQ ID NO: 318 - DNA, 29 bp, Homo sapiens
  modified_base (15)..(15): a, c, g, t, unknown or other
ctgttctgtgatatngattttttaagcaa

SEQ ID NO: 319 - DNA, 29 bp, Homo sapiens
  modified_base (15)..(15): a, c, g, t, unknown or other
tgaaaaggaaaacanagggtggcagagga

SEQ ID NO: 320 - DNA, 29 bp, Homo sapiens
  modified_base (15)..(15): a, c, g, t, unknown or other
tttaacaactaatanccaatccatgaggg

SEQ ID NO: 321 - DNA, 29 bp, Homo sapiens
  modified_base (15)..(15): a, c, g, t, unknown or other
acaaattgtgacccncgtaggcaatatgc

SEQ ID NO: 322 - DNA, 29 bp, Homo sapiens
  modified_base (15)..(15): a, c, g, t, unknown or other
tactcaacatgttanttcctcctaagcta

SEQ ID NO: 323 - DNA, 29 bp, Homo sapiens
  modified_base (15)..(15): a, c, g, t, unknown or other
gttctgatactaagnctgaaagattcatg

SEQ ID NO: 324 - DNA, 29 bp, Homo sapiens
  modified_base (15)..(15): a, c, g, t, unknown or other
tctgaaatcttagtncaacttcctcaccc

SEQ ID NO: 325 - DNA, 29 bp, Homo sapiens
  modified_base (15)..(15): a, c, g, t, unknown or other
tttcaactacacttnttacccatctctca

SEQ ID NO: 326 - DNA, 29 bp, Homo sapiens
  modified_base (15)..(15): a, c, g, t, unknown or other
aacataaattatacngtggcaggttggta

SEQ ID NO: 327 - DNA, 29 bp, Homo sapiens
  modified_base (15)..(15): a, c, g, t, unknown or other
gatccttatgtgccnattcgtaccaccct

SEQ ID NO: 328 - DNA, 29 bp, Homo sapiens
  modified_base (15)..(15): a, c, g, t, unknown or other
acagcagaagccgcnatgctgtttctgaa

SEQ ID NO: 329 - DNA, 29 bp, Homo sapiens
  modified_base (15)..(15): a, c, g, t, unknown or other
gccacggtgttcatntggacagaaacaac

SEQ ID NO: 330 - DNA, 29 bp, Homo sapiens
  modified_base (15)..(15): a, c, g, t, unknown or other
ttatcccatcaaaantcattcatcacaac

SEQ ID NO: 331 - DNA, 29 bp, Homo sapiens
  modified_base (15)..(15): a, c, g, t, unknown or other
attaggtacgcagangaaactcttgttca

SEQ ID NO: 332 - DNA, 29 bp, Homo sapiens
  modified_base (15)..(15): a, c, g, t, unknown or other
tttaaatgtgacaantcttggaagaatta

SEQ ID NO: 333 - DNA, 29 bp, Homo sapiens
  modified_base (15)..(15): a, c, g, t, unknown or other
tgactttattttccnctaaatgtgtgtcc

SEQ ID NO: 334 - DNA, 29 bp, Homo sapiens
  modified_base (15)..(15): a, c, g, t, unknown or other
actgcacagatggangacccaaaccagca

SEQ ID NO: 335 - DNA, 29 bp, Homo sapiens
  modified_base (15)..(15): a, c, g, t, unknown or other
actctaaattctacncgataaggtgcatg

SEQ ID NO: 336 - DNA, 29 bp, Homo sapiens
  modified_base (15)..(15): a, c, g, t, unknown or other
ggtaggatgggacanatcatggaatttgg

SEQ ID NO: 337 - DNA, 29 bp, Homo sapiens
  modified_base (15)..(15): a, c, g, t, unknown or other
ttctcccgagctgangagtatgtttttcc

SEQ ID NO: 338 - DNA, 29 bp, Homo sapiens
  modified_base (15)..(15): a, c, g, t, unknown or other
tagatgcttcccagncgattctcccgtga

SEQ ID NO: 339 - DNA, 29 bp, Homo sapiens
  modified_base (15)..(15): a, c, g, t, unknown or other
gtgggttggccttcnatcctctagctgtc

SEQ ID NO: 340 - DNA, 29 bp, Homo sapiens
  modified_base (15)..(15): a, c, g, t, unknown or other
ttttcactatttcantcatggcctagtct

SEQ ID NO: 341 - DNA, 29 bp, Homo sapiens
  modified_base (15)..(15): a, c, g, t, unknown or other
tttgcatgatgacantcagtgatcacagt

SEQ ID NO: 342 - DNA, 29 bp, Homo sapiens
  modified_base (15)..(15): a, c, g, t, unknown or other
ttctgtggacactcnatttgacggcagaa

SEQ ID NO: 343 - DNA, 29 bp, Homo sapiens
  modified_base (15)..(15): a, c, g, t, unknown or other
taaaatagaaataantttttattgccatt

SEQ ID NO: 344 - DNA, 29 bp, Homo sapiens
  modified_base (15)..(15): a, c, g, t, unknown or other
gggccatctgttccnaggctggttggaga

SEQ ID NO: 345 - DNA, 29 bp, Homo sapiens
  modified_base (15)..(15): a, c, g, t, unknown or other
gtctgtcatgcaccnagttgatggtcttc

SEQ ID NO: 346 - DNA, 29 bp, Homo sapiens
  modified_base (15)..(15): a, c, g, t, unknown or other
aggtcattgtgtctntggtgattaaacca

SEQ ID NO: 347 - DNA, 29 bp, Homo sapiens
  modified_base (15)..(15): a, c, g, t, unknown or other
ccagtgcagttccangtctcagtgtatat

SEQ ID NO: 348 - DNA, 29 bp, Homo sapiens
  modified_base (15)..(15): a, c, g, t, unknown or other
ccaggctccatacanctgccattttattc

SEQ ID NO: 349 - DNA, 29 bp, Homo sapiens
  modified_base (15)..(15): a, c, g, t, unknown or other
agtaggaggaatgcnggttgaaagggatg

SEQ ID NO: 350 - DNA, 29 bp, Homo sapiens
  modified_base (15)..(15): a, c, g, t, unknown or other
tcttggtcctaagangtctgtctgcagca

SEQ ID NO: 351 - DNA, 29 bp, Homo sapiens
  modified_base (15)..(15): a, c, g, t, unknown or other
ggcagccatctcccnggattgggcaatgc

SEQ ID NO: 352 - DNA, 29 bp, Homo sapiens
  modified_base (15)..(15): a, c, g, t, unknown or other
cagtgaaggtctagngattctcccctaaa

SEQ ID NO: 353 - DNA, 29 bp, Homo sapiens
  modified_base (15)..(15): a, c, g, t, unknown or other
gctgcagatgagagnttctcccaggtgga

SEQ ID NO: 354 - DNA, 29 bp, Homo sapiens
  modified_base (15)..(15): a, c, g, t, unknown or other
ctgtctgggtgttanagaaacaaacatcc

SEQ ID NO: 355 - DNA, 29 bp, Homo sapiens
  modified_base (15)..(15): a, c, g, t, unknown or other
atgatggttataacntgggtatggagcac

SEQ ID NO: 356 - DNA, 29 bp, Homo sapiens
  modified_base (15)..(15): a, c, g, t, unknown or other
tctggcagactttcnttcagtggccagga

SEQ ID NO: 357 - DNA, 29 bp, Homo sapiens
  modified_base (15)..(15): a, c, g, t, unknown or other
tattttcacagtaanagtcactaaagggg

SEQ ID NO: 358 - DNA, 29 bp, Homo sapiens
  modified_base (15)..(15): a, c, g, t, unknown or other
tcctgcctcagagtngagatctgcagttt

SEQ ID NO: 359 - DNA, 29 bp, Homo sapiens
  modified_base (15)..(15): a, c, g, t, unknown or other
catctgggaggttgntaacagtgagctct

SEQ ID NO: 360 - DNA, 29 bp, Homo sapiens
  modified_base (15)..(15): a, c, g, t, unknown or other
agcccgaaataatgncaagtggattcatg

SEQ ID NO: 361 - DNA, 29 bp, Homo sapiens
  modified_base (15)..(15): a, c, g, t, unknown or other
aagtctgatggaaanagtgattctgaagg

SEQ ID NO: 362 - DNA, 29 bp, Homo sapiens
  modified_base (15)..(15): a, c, g, t, unknown or other
aaacacggatggacntcgccagaatttaa

SEQ ID NO: 363 - DNA, 29 bp, Homo sapiens
  modified_base (15)..(15): a, c, g, t, unknown or other
atggaattgggaaangtgaattaccttta

SEQ ID NO: 364 - DNA, 29 bp, Homo sapiens
  modified_base (15)..(15): a, c, g, t, unknown or other
cattggcatctgcangtcttcttgttaat

SEQ ID NO: 365 - DNA, 29 bp, Homo sapiens
  modified_base (15)..(15): a, c, g, t, unknown or other
cttgcaaagtgctanatccactatcactg

SEQ ID NO: 366 - DNA, 29 bp, Homo sapiens
  modified_base (15)..(15): a, c, g, t, unknown or other
attgccgaagtatangcccagggactcac

SEQ ID NO: 367 - DNA, 29 bp, Homo sapiens
  modified_base (15)..(15): a, c, g, t, unknown or other
acagaattgcacagngaggccctagcaac

SEQ ID NO: 368 - DNA, 29 bp, Homo sapiens
  modified_base (15)..(15): a, c, g, t, unknown or other
taggttccatgaccncacggctttcacag

SEQ ID NO: 369 - DNA, 29 bp, Homo sapiens
  modified_base (15)..(15): a, c, g, t, unknown or other
taccctgctattccngaacctgcccatct

SEQ ID NO: 370 - DNA, 29 bp, Homo sapiens
  modified_base (15)..(15): a, c, g, t, unknown or other
ggcatgaagccctcnctttttatcccatt

SEQ ID NO: 371 - DNA, 29 bp, Homo sapiens
  modified_base (15)..(15): a, c, g, t, unknown or other
aaatgtctgctggcnaagatgtggtcccc

SEQ ID NO: 372 - DNA, 29 bp, Homo sapiens
  modified_base (15)..(15): a, c, g, t, unknown or other
agacactcctgcagngaaccaatttttct

SEQ ID NO: 373 - DNA, 29 bp, Homo sapiens
  modified_base (15)..(15): a, c, g, t, unknown or other
attcatttgaaaagngcttggatggttta

SEQ ID NO: 374 - DNA, 29 bp, Homo sapiens
  modified_base (15)..(15): a, c, g, t, unknown or other
ttaaagggtcagtcnttgaaacattgctg

SEQ ID NO: 375 - DNA, 29 bp, Homo sapiens
  modified_base (15)..(15): a, c, g, t, unknown or other
tctgggatgcaggtngtggttgccttgca

SEQ ID NO: 376 - DNA, 29 bp, Homo sapiens
  modified_base (15)..(15): a, c, g, t, unknown or other
gttcctgtgatgccntcaaaatgagtaag

SEQ ID NO: 377 - DNA, 29 bp, Homo sapiens
  modified_base (15)..(15): a, c, g, t, unknown or other
aagacctgactaccnctgcgggattccct

SEQ ID NO: 378 - DNA, 29 bp, Homo sapiens
  modified_base (15)..(15): a, c, g, t, unknown or other
catacaattcaaacntggtcattgtcatt

SEQ ID NO: 379 - DNA, 29 bp, Homo sapiens
  modified_base (15)..(15): a, c, g, t, unknown or other
gcaccatccagagcnctccaaggacactc

SEQ ID NO: 380 - DNA, 29 bp, Homo sapiens
  modified_base (15)..(15): a, c, g, t, unknown or other
gtcaagcaaaggagngtgtcttattcgtg

SEQ ID NO: 381 - DNA, 29 bp, Homo sapiens
  modified_base (15)..(15): a, c, g, t, unknown or other
tagagtagctgttgnccagtttcatggtc

SEQ ID NO: 382 - DNA, 29 bp, Homo sapiens
  modified_base (15)..(15): a, c, g, t, unknown or other
tattccagctgtatngcagaaatatttta

SEQ ID NO: 383 - DNA, 29 bp, Homo sapiens
  modified_base (15)..(15): a, c, g, t, unknown or other
gctgtaaggctacantgagaatttacgaa

SEQ ID NO: 384 - DNA, 29 bp, Homo sapiens
  modified_base (15)..(15): a, c, g, t, unknown or other
acttctttagcatanaattcagatacaac

SEQ ID NO: 385 - DNA, 29 bp, Homo sapiens
  modified_base (15)..(15): a, c, g, t, unknown or other
ttttgtgtcctaacntgatctatcctgga

SEQ ID NO: 386 - DNA, 29 bp, Homo sapiens
  modified_base (15)..(15): a, c, g, t, unknown or other
acctattgtcctcantgctacaggtatta

SEQ ID NO: 387 - DNA, 29 bp, Homo sapiens
  modified_base (15)..(15): a, c, g, t, unknown or other
agagaattggagtcngttaagcaatgggt

SEQ ID NO: 388 - DNA, 29 bp, Homo sapiens
  modified_base (15)..(15): a, c, g, t, unknown or other
aggaagacagacaantaaacaagtacaat

SEQ ID NO: 389 - DNA, 29 bp, Homo sapiens
  modified_base (15)..(15): a, c, g, t, unknown or other
agagacacagtgaantccaactaaatgtc

SEQ ID NO: 390 - DNA, 29 bp, Homo sapiens
  modified_base (15)..(15): a, c, g, t, unknown or other
ctgaaatccccactngaatgagaggaaat

SEQ ID NO: 391 - DNA, 29 bp, Homo sapiens
  modified_base (15)..(15): a, c, g, t, unknown or other
agttctggggatcangggccttgtgaaaa

SEQ ID NO: 392 - DNA, 29 bp, Homo sapiens
  modified_base (15)..(15): a, c, g, t, unknown or other
gatacttgaattatngtgcgcctgtgagc

SEQ ID NO: 393 - DNA, 29 bp, Homo sapiens
  modified_base (15)..(15): a, c, g, t, unknown or other
cctcatttgtcaaantggattgatgataa

SEQ ID NO: 394 - DNA, 29 bp, Homo sapiens
  modified_base (15)..(15): a, c, g, t, unknown or other
tatctcatcctggtntcttctctccatat

SEQ ID NO: 395 - DNA, 29 bp, Homo sapiens
  modified_base (15)..(15): a, c, g, t, unknown or other
ccatggcagtatatngagctattttctca

SEQ ID NO: 396 - DNA, 29 bp, Homo sapiens
  modified_base (15)..(15): a, c, g, t, unknown or other
gggacataaagaaangttgagtgcacgac

SEQ ID NO: 397 - DNA, 29 bp, Homo sapiens
  modified_base (15)..(15): a, c, g, t, unknown or other
agttcgaaagaagangcaagcgctggcct

SEQ ID NO: 398 - DNA, 29 bp, Homo sapiens
  modified_base (15)..(15): a, c, g, t, unknown or other
ttgaaagtgaccttnggccatggactgtt

SEQ ID NO: 399 - DNA, 29 bp, Homo sapiens
  modified_base (15)..(15): a, c, g, t, unknown or other
cagcactggctgtgnactgtacccgtggg

SEQ ID NO: 400 - DNA, 29 bp, Homo sapiens
  modified_base (15)..(15): a, c, g, t, unknown or other
cccaaagcaaagtcnttaataatattcac

SEQ ID NO: 401 - DNA, 29 bp, Homo sapiens
  modified_base (15)..(15): a, c, g, t, unknown or other
gggcctctttgccgngaggcattttgtgc

SEQ ID NO: 402 - DNA, 29 bp, Homo sapiens
  modified_base (15)..(15): a, c, g, t, unknown or other
ccgatatcaagtgtngtgtgtcatctccc

SEQ ID NO: 403 - DNA, 29 bp, Homo sapiens
  modified_base (15)..(15): a, c, g, t, unknown or other
gggacaagagcaatngccgtacctcaggc

SEQ ID NO: 404 - DNA, 29 bp, Homo sapiens
  modified_base (15)..(15): a, c, g, t, unknown or other
tctgggctgttcttngcggagaacggaaa

SEQ ID NO: 405 - DNA, 29 bp, Homo sapiens
  modified_base (15)..(15): a, c, g, t, unknown or other
gggacggagcagggnccactgagtcaacc

SEQ ID NO: 406 - DNA, 29 bp, Homo sapiens
  modified_base (15)..(15): a, c, g, t, unknown or other
ccttttcaaatcgtnttgcacaccttacc

SEQ ID NO: 407 - DNA, 29 bp, Homo sapiens
  modified_base (15)..(15): a, c, g, t, unknown or other
aataacaaaaatgantgctggtgtcgtta

SEQ ID NO: 408 - DNA, 29 bp, Homo sapiens
  modified_base (15)..(15): a, c, g, t, unknown or other
cttattttacagacncaaaagctcaccag

SEQ ID NO: 409 - DNA, 29 bp, Homo sapiens
  modified_base (15)..(15): a, c, g, t, unknown or other
ctttgtttgtcagtngcctgttgcccacc

SEQ ID NO: 410 - DNA, 29 bp, Homo sapiens
  modified_base (15)..(15): a, c, g, t, unknown or other
gactctccaccttcnggcacagagggcac

SEQ ID NO: 411 - DNA, 29 bp, Homo sapiens
  modified_base (15)..(15): a, c, g, t, unknown or other
atggatttatggttnaactggtctaatcc

SEQ ID NO: 412 - DNA, 29 bp, Homo sapiens
  modified_base (15)..(15): a, c, g, t, unknown or other
agtgtttcagaggcnagtggtgttttgtg

SEQ ID NO: 413 - DNA, 29 bp, Homo sapiens
  modified_base (15)..(15): a, c, g, t, unknown or other
aggaaatagggtgtnggtttgctataaag

SEQ ID NO: 414 - DNA, 29 bp, Homo sapiens
  modified_base (15)..(15): a, c, g, t, unknown or other
gacttttctacccancctcttgtcacaaa

SEQ ID NO: 415 - DNA, 29 bp, Homo sapiens
  modified_base (15)..(15): a, c, g, t, unknown or other
tttaatttaacaatnagacataaaacatt

SEQ ID NO: 416 - DNA, 29 bp, Homo sapiens
  modified_base (15)..(15): a, c, g, t, unknown or other
tttaagaaatagttngtatgtttactaat

SEQ ID NO: 417 - DNA, 29 bp, Homo sapiens
  modified_base (15)..(15): a, c, g, t, unknown or other
tcttttgatcattangtttcctgttttca

SEQ ID NO: 418 - DNA, 29 bp, Homo sapiens
  modified_base (15)..(15): a, c, g, t, unknown or other
aaataaaggatttcngttggatttctagc

SEQ ID NO: 419 - DNA, 29 bp, Homo sapiens
  modified_base (15)..(15): a, c, g, t, unknown or other
tattgttttccattncaaggtgtgtattc

SEQ ID NO: 420 - DNA, 29 bp, Homo sapiens
  modified_base (15)..(15): a, c, g, t, unknown or other
aacacctaaagtagntgaagaggttaatg

SEQ ID NO: 421 - DNA, 29 bp, Homo sapiens
  modified_base (15)..(15): a, c, g, t, unknown or other
tggggtgacagaagnattctgcatcatga

SEQ ID NO: 422 - DNA, 29 bp, Homo sapiens
  modified_base (15)..(15): a, c, g, t, unknown or other
cttttttgaacctcngttgctggggcatt

SEQ ID NO: 423 - DNA, 29 bp, Homo sapiens
  modified_base (15)..(15): a, c, g, t, unknown or other
cccaaatttttagantacagccaaagtaa

SEQ ID NO: 424 - DNA, 29 bp, Homo sapiens
  modified_base (15)..(15): a, c, g, t, unknown or other
ctgccttttgccttnggtgaactggctag

SEQ ID NO: 425 - DNA, 29 bp, Homo sapiens
  modified_base (15)..(15): a, c, g, t, unknown or other
tatctgaacccgtanaaagatttagtaat

SEQ ID NO: 426 - DNA, 29 bp, Homo sapiens
  modified_base (15)..(15): a, c, g, t, unknown or other
ctacttcagtaattnaatgtgcttgcaga

SEQ ID NO: 427 - DNA, 29 bp, Homo sapiens
  modified_base (15)..(15): a, c, g, t, unknown or other
aaatgttaaaagtanattttgtctcattg

SEQ ID NO: 428 - DNA, 29 bp, Homo sapiens
  modified_base (15)..(15): a, c, g, t, unknown or other
ttcaagtttaaggtnctcaatatttttaa

SEQ ID NO: 429 - DNA, 29 bp, Homo sapiens
  modified_base (15)..(15): a, c, g, t, unknown or other
agaatggcctctctnttgcagatattcac

SEQ ID NO: 430 - DNA, 29 bp, Homo sapiens
  modified_base (15)..(15): a, c, g, t, unknown or other
ctaaagcattatatnccgcctatacattt

SEQ ID NO: 431 - DNA, 29 bp, Homo sapiens
  modified_base (15)..(15): a, c, g, t, unknown or other
gctgtattaaccaantttgataaatagaa

SEQ ID NO: 432 - DNA, 29 bp, Homo sapiens
  modified_base (15)..(15): a, c, g, t, unknown or other
aactgcagttaagtnagggagttaagtca

SEQ ID NO: 433 - DNA, 29 bp, Homo sapiens
  modified_base (15)..(15): a, c, g, t, unknown or other
ataagtggacttganttacctaatgtttt

SEQ ID NO: 434 - DNA, 29 bp, Homo sapiens
  modified_base (15)..(15): a, c, g, t, unknown or other
cgcaaacttcttatnagattctgattcca

SEQ ID NO: 435 - DNA, 29 bp, Homo sapiens
  modified_base (15)..(15): a, c, g, t, unknown or other
tgcttatatggatcngactaccctgtcct

SEQ ID NO: 436 - DNA, 29 bp, Homo sapiens
  modified_base (15)..(15): a, c, g, t, unknown or other
ctaaaaaaaataagntaactatcctgacg

SEQ ID NO: 437 - DNA, 29 bp, Homo sapiens
  modified_base (15)..(15): a, c, g, t, unknown or other
agttttaaagcagcnaagtcatgtttctt

SEQ ID NO: 438 - DNA, 29 bp, Homo sapiens
  modified_base (15)..(15): a, c, g, t, unknown or other
gagtttctttcagtngatcaacaacgcaa

SEQ ID NO: 439 - DNA, 29 bp, Homo sapiens
  modified_base (15)..(15): a, c, g, t, unknown or other
tcacagtacatccanttgagaaggcctcc

SEQ ID NO: 440 - DNA, 29 bp, Homo sapiens
  modified_base (15)..(15): a, c, g, t, unknown or other
tctatgaaacaacgntaaaccacttattc

SEQ ID NO: 441 - DNA, 29 bp, Homo sapiens
  modified_base (15)..(15): a, c, g, t, unknown or other
gtaaaaatcaaattntgtgaagctgctaa

SEQ ID NO: 442 - DNA, 29 bp, Homo sapiens
  modified_base (15)..(15): a, c, g, t, unknown or other
caggtgcaatggatntactcttaatcctc

SEQ ID NO: 443 - DNA, 29 bp, Homo sapiens
  modified_base (15)..(15): a, c, g, t, unknown or other
actcagaataattcntgattttatgatag

SEQ ID NO: 444 - DNA, 29 bp, Homo sapiens
  modified_base (15)..(15): a, c, g, t, unknown or other
tctgagattcagaangcagcaaacttata

SEQ ID NO: 445 - DNA, 29 bp, Homo sapiens
  modified_base (15)..(15): a, c, g, t, unknown or other
ttttggacaatttcnaatacactgcataa

SEQ ID NO: 446 - DNA, 29 bp, Homo sapiens
  modified_base (15)..(15): a, c, g, t, unknown or other
tcggtatgaaggaanagaatgctgagaat

SEQ ID NO: 447 - DNA, 29 bp, Homo sapiens
  modified_base (15)..(15): a, c, g, t, unknown or other
ttcggtcttattttngggtaaatttatac

SEQ ID NO: 448 - DNA, 29 bp, Homo sapiens
  modified_base (15)..(15): a, c, g, t, unknown or other
agtgtgatgcttagntacaaaaattttgg

SEQ ID NO: 449 - DNA, 29 bp, Homo sapiens
  modified_base (15)..(15): a, c, g, t, unknown or other
aaactccatcctatntaactgccctgttt

SEQ ID NO: 450 - DNA, 29 bp, Homo sapiens
  modified_base (15)..(15): a, c, g, t, unknown or other
gaaattacttcaggncaaagtggaaggga

SEQ ID NO: 451 - DNA, 29 bp, Homo sapiens
  modified_base (15)..(15): a, c, g, t, unknown or other
gacgaattctacagngacctgaaagctat

SEQ ID NO: 452 - DNA, 29 bp, Homo sapiens
  modified_base (15)..(15): a, c, g, t, unknown or other
tcctcagaaattccntcaaaatttacatc

SEQ ID NO: 453 - DNA, 29 bp, Homo sapiens
  modified_base (15)..(15): a, c, g, t, unknown or other
tgttagctattactnttaattttatcata

SEQ ID NO: 454 - DNA, 29 bp, Homo sapiens
  modified_base (15)..(15): a, c, g, t, unknown or other
taataaattatgatntgctctctaattat

SEQ ID NO: 455 - DNA, 29 bp, Homo sapiens
  modified_base (15)..(15): a, c, g, t, unknown or other
gtgctgtcttttcanctgcaaattcctag

SEQ ID NO: 456 - DNA, 29 bp, Homo sapiens
  modified_base (15)..(15): a, c, g, t, unknown or other
atattatataaaganttttctgcttttaa

SEQ ID NO: 457 - DNA, 29 bp, Homo sapiens
  modified_base (15)..(15): a, c, g, t, unknown or other
taatttgtgattcantgtttgtacttctg

SEQ ID NO: 458 - DNA, 29 bp, Homo sapiens
  modified_base (15)..(15): a, c, g, t, unknown or other
gccagctgaggaaanatggaacacctaat

SEQ ID NO: 459 - DNA, 29 bp, Homo sapiens
  modified_base (15)..(15): a, c, g, t, unknown or other
catttcttggtgaanaaaatagaactata

SEQ ID NO: 460 - DNA, 29 bp, Homo sapiens
  modified_base (15)..(15): a, c, g, t, unknown or other
tgaaaatcctagaantaattcaaaacaat

SEQ ID NO: 461 - DNA, 29 bp, Homo sapiens
  modified_base (15)..(15): a, c, g, t, unknown or other
atttaaaatgcttgntgttttgagaaact

SEQ ID NO: 462 - DNA, 29 bp, Homo sapiens
  modified_base (15)..(15): a, c, g, t, unknown or other
tttagggtgatacanatatacttttatag

SEQ ID NO: 463 - DNA, 29 bp, Homo sapiens
  modified_base (15)..(15): a, c, g, t, unknown or other
ataaaggagcatatntttttctacagcag

SEQ ID NO: 464 - DNA, 29 bp, Homo sapiens
  modified_base (15)..(15): a, c, g, t, unknown or other
gtaaattgaaaagtnacataattggtact

SEQ ID NO: 465 - DNA, 29 bp, Homo sapiens
  modified_base (15)..(15): a, c, g, t, unknown or other
tttgactggaggaanaaaatagagcaaaa

SEQ ID NO: 466 - DNA, 29 bp, Homo sapiens
  modified_base (15)..(15): a, c, g, t, unknown or other
ctctgcagaaaaagntatgatgcttaaca

SEQ ID NO: 467 - DNA, 29 bp, Homo sapiens
  modified_base (15)..(15): a, c, g, t, unknown or other
acttaataatattcnttcactaaacacaa

SEQ ID NO: 468 - DNA, 29 bp, Homo sapiens
  modified_base (15)..(15): a, c, g, t, unknown or other
atgactggtgggatnaattgaatttattt

SEQ ID NO: 469 - DNA, 29 bp, Homo sapiens
  modified_base (15)..(15): a, c, g, t, unknown or other
gctacctcttcttgnaaaggacctattta

SEQ ID NO: 470 - DNA, 29 bp, Homo sapiens
  modified_base (15)..(15): a, c, g, t, unknown or other
tgcaggagtttgtantctcagggagctgc

SEQ ID NO: 471 - DNA, 29 bp, Homo sapiens
  modified_base (15)..(15): a, c, g, t, unknown or other
gggacaaagttcctntctactcccattgt

SEQ ID NO: 472 - DNA, 29 bp, Homo sapiens
  modified_base (15)..(15): a, c, g, t, unknown or other
agcattatttctgangacaaaagcaatct

SEQ ID NO: 473 - DNA, 29 bp, Homo sapiens
  modified_base (15)..(15): a, c, g, t, unknown or other
cactcactagttctnatgccatttctaag

SEQ ID NO: 474 - DNA, 29 bp, Homo sapiens
  modified_base (15)..(15): a, c, g, t, unknown or other
acaggctttaatttnctaaaagtctagac

SEQ ID NO: 475 - DNA, 29 bp, Homo sapiens
  modified_base (15)..(15): a, c, g, t, unknown or other
ccaatgggacataanccctggggaaagac

SEQ ID NO: 476 - DNA, 29 bp, Homo sapiens
  modified_base (15)..(15): a, c, g, t, unknown or other
tattttcaatcatgncttttttctgttta

SEQ ID NO: 477 - DNA, 29 bp, Homo sapiens
  modified_base (15)..(15): a, c, g, t, unknown or other
actctaagtattccnaacatgaaaggctc

SEQ ID NO: 478 - DNA, 29 bp, Homo sapiens
  modified_base (15)..(15): a, c, g, t, unknown or other
cataaactctaatgnatctaatttatttc

SEQ ID NO: 479 - DNA, 29 bp, Homo sapiens
  modified_base (15)..(15): a, c, g, t, unknown or other
gtgatctcgaaggancccatgctttgctg

SEQ ID NO: 480 - DNA, 29 bp, Homo sapiens
  modified_base (15)..(15): a, c, g, t, unknown or other
aggttctgtctcgtngcattgtattcctt

SEQ ID NO: 481 - DNA, 29 bp, Homo sapiens
  modified_base (15)..(15): a, c, g, t, unknown or other
atgactgactgtacnatgagccacatact

SEQ ID NO: 482 - DNA, 29 bp, Homo sapiens
  modified_base (15)..(15): a, c, g, t, unknown or other
gtcctatgactgcantggtccccaaaaag

SEQ ID NO: 483 - DNA, 29 bp, Homo sapiens
  modified_base (15)..(15): a, c, g, t, unknown or other
aggacacggggtcanttagggcaatagtg

SEQ ID NO: 484 - DNA, 29 bp, Homo sapiens
  modified_base (15)..(15): a, c, g, t, unknown or other
ggtatatagcagtgncttagtaaatgctg

SEQ ID NO: 485 - DNA, 29 bp, Homo sapiens
  modified_base (15)..(15): a, c, g, t, unknown or other
ttgcctgaaccttangctttgggattcac

SEQ ID NO: 486 - DNA, 29 bp, Homo sapiens
  modified_base (15)..(15): a, c, g, t, unknown or other
cgtgacaggatcctnaggggcaactctat

SEQ ID NO: 487 - DNA, 29 bp, Homo sapiens
  modified_base (15)..(15): a, c, g, t, unknown or other
aaatttttaactaanaagttctctgttgt

SEQ ID NO: 488 - DNA, 29 bp, Homo sapiens
  modified_base (15)..(15): a, c, g, t, unknown or other
aatggtaatgaatgntcttcaatcagtat

SEQ ID NO: 489 - DNA, 29 bp, Homo sapiens
  modified_base (15)..(15): a, c, g, t, unknown or other
cagagagatatctantaagggtaaaaaag

SEQ ID NO: 490 - DNA, 29 bp, Homo sapiens
  modified_base (15)..(15): a, c, g, t, unknown or other
gtacttgacattcantttaacgtgcttaa

SEQ ID NO: 491 - DNA, 29 bp, Homo sapiens
  modified_base (15)..(15): a, c, g, t, unknown or other
acctgctggttttanggagaaggcagggg

SEQ ID NO: 492 - DNA, 29 bp, Homo sapiens
  modified_base (15)..(15): a, c, g, t, unknown or other
tttctccataggttntatcttgacagaaa

SEQ ID NO: 493 - DNA, 29 bp, Homo sapiens
  modified_base (15)..(15): a, c, g, t, unknown or other
ttgctttaaatatanagcaagtagagctc

SEQ ID NO: 494 - DNA, 29 bp, Homo sapiens
  modified_base (15)..(15): a, c, g, t, unknown or other
tgattttgttcctantgtgtcgatgtgcg

SEQ ID NO: 495 - DNA, 29 bp, Homo sapiens
  modified_base (15)..(15): a, c, g, t, unknown or other
gataactcccttcantccctagaaactta

SEQ ID NO: 496 - DNA, 29 bp, Homo sapiens
  modified_base (15)..(15): a, c, g, t, unknown or other
atttgtccaggttanatctatagagttct

SEQ ID NO: 497 - DNA, 29 bp, Homo sapiens
  modified_base (15)..(15): a, c, g, t, unknown or other
gcagacatcaaacanttcaagggactcag

SEQ ID NO: 498 - DNA, 29 bp, Homo sapiens
  modified_base (15)..(15): a, c, g, t, unknown or other
tgaatacaccatcantgtgggtgacatgg

SEQ ID NO: 499 - DNA, 29 bp, Homo sapiens
  modified_base (15)..(15): a, c, g, t, unknown or other
taagagatgtaaaangaaatgaatagaag

SEQ ID NO: 500 - DNA, 29 bp, Homo sapiens
  modified_base (15)..(15): a, c, g, t, unknown or other
aaataattagtaatntatgcagacgtgtc

SEQ ID NO: 501 - DNA, 29 bp, Homo sapiens
  modified_base (15)..(15): a, c, g, t, unknown or other
ttgacttcctgtaangtgtatggtggtat

SEQ ID NO: 502 - DNA, 29 bp, Homo sapiens
  modified_base (15)..(15): a, c, g, t, unknown or other
cactggaagttacantcatcattatgtgg

SEQ ID NO: 503 - DNA, 29 bp, Homo sapiens
  modified_base (15)..(15): a, c, g, t, unknown or other
ttatttgcaaaaagnctatcataagatga

SEQ ID NO: 504 - DNA, 29 bp, Homo sapiens
  modified_base (15)..(15): a, c, g, t, unknown or other
ctcaaagaccacccnatgtcattttctac

SEQ ID NO: 505 - DNA, 29 bp, Homo sapiens
  modified_base (15)..(15): a, c, g, t, unknown or other
aaattagaatgtgcntagcgttttgtcgt

SEQ ID NO: 506 - DNA, 29 bp, Homo sapiens
  modified_base (15)..(15): a, c, g, t, unknown or other
gaattaaaacaccanactcataagcactg

SEQ ID NO: 507 - DNA, 29 bp, Homo sapiens
  modified_base (15)..(15): a, c, g, t, unknown or other
cacatatttcagccntcaactcctaattt

SEQ ID NO: 508 - DNA, 29 bp, Homo sapiens
  modified_base (15)..(15): a, c, g, t, unknown or other
atataaaaacgttgnagtgaactgaaata

SEQ ID NO: 509 - DNA, 29 bp, Homo sapiens
  modified_base (15)..(15): a, c, g, t, unknown or other
gcaagggataacacnaaatggtgcaatta

SEQ ID NO: 510 - DNA, 29 bp, Homo sapiens
  modified_base (15)..(15): a, c, g, t, unknown or other
acttgattttttttnctgaaaattaggta

SEQ ID NO: 511 - DNA, 29 bp, Homo sapiens
  modified_base (15)..(15): a, c, g, t, unknown or other
gataagaataaaacngcacagcaacaatt

SEQ ID NO: 512 - DNA, 29 bp, Homo sapiens
  modified_base (15)..(15): a, c, g, t, unknown or other
gctgcttctgcaaanccatgacctaaatc

SEQ ID NO: 513 - DNA, 29 bp, Homo sapiens
  modified_base (15)..(15): a, c, g, t, unknown or other
tggcaggaatcagancgtgaatacactta

SEQ ID NO: 514 - DNA, 29 bp, Homo sapiens
  modified_base (15)..(15): a, c, g, t, unknown or other
gaagactgaataatngcatggatatttag

SEQ ID NO: 515 - DNA, 29 bp, Homo sapiens
  modified_base (15)..(15): a, c, g, t, unknown or other
aaatttgtaccacantccacattcattaa

SEQ ID NO: 516 - DNA, 29 bp, Homo sapiens
  modified_base (15)..(15): a, c, g, t, unknown or other
aatttgaataaaacntcttgatgaaatga

SEQ ID NO: 517 - DNA, 29 bp, Homo sapiens
  modified_base (15)..(15): a, c, g, t, unknown or other
cctttcatgtaagtnattgagatggtgag

SEQ ID NO: 518 - DNA, 29 bp, Homo sapiens
  modified_base (15)..(15): a, c, g, t, unknown or other
catgcaaccagtaantgacagatctcact

SEQ ID NO: 519 - DNA, 29 bp, Homo sapiens
  modified_base (15)..(15): a, c, g, t, unknown or other
cccagaggttcattncaatttttggccaa

SEQ ID NO: 520 - DNA, 29 bp, Homo sapiens
  modified_base (15)..(15): a, c, g, t, unknown or other
agatttgtttggtanatattagctataaa

SEQ ID NO: 521 - DNA, 29 bp, Homo sapiens
  modified_base (15)..(15): a, c, g, t, unknown or other
tgtgtgctaaataantgaatgagtgaaaa

SEQ ID NO: 522 - DNA, 29 bp, Homo sapiens
  modified_base (15)..(15): a, c, g, t, unknown or other
acatgtcaatgggtntgacgctgacactt

SEQ ID NO: 523 - DNA, 29 bp, Homo sapiens
  modified_base (15)..(15): a, c, g, t, unknown or other
tgttacagatgtganacagggtagattct

SEQ ID NO: 524 - DNA, 29 bp, Homo sapiens
  modified_base (15)..(15): a, c, g, t, unknown or other
atttttacttttcantgtggctgatttta

SEQ ID NO: 525 - DNA, 29 bp, Homo sapiens
  modified_base (15)..(15): a, c, g, t, unknown or other
ctccttaggacacanttgaatgcctagaa

SEQ ID NO: 526 - DNA, 29 bp, Homo sapiens
  modified_base (15)..(15): a, c, g, t, unknown or other
tggttgaataatcantgttctggccaaat

SEQ ID NO: 527 - DNA, 29 bp, Homo sapiens
  modified_base (15)..(15): a, c, g, t, unknown or other
ttaaagagatcacanaagcatgtagcata

SEQ ID NO: 528 - DNA, 29 bp, Homo sapiens
  modified_base (15)..(15): a, c, g, t, unknown or other
tgcgtagagaagatnctcagtcagcagca

SEQ ID NO: 529 - DNA, 29 bp, Homo sapiens
  modified_base (15)..(15): a, c, g, t, unknown or other
tggcacaatctctcnggattcctgattca

SEQ ID NO: 530 - DNA, 29 bp, Homo sapiens
  modified_base (15)..(15): a, c, g, t, unknown or other
ttacttcgagaaaanggaaagggaaacac

SEQ ID NO: 531 - DNA, 29 bp, Homo sapiens
  modified_base (15)..(15): a, c, g, t, unknown or other
catctctaagttgcnttccactttttaga

SEQ ID NO: 532 - DNA, 29 bp, Homo sapiens
  modified_base (15)..(15): a, c, g, t, unknown or other
agatgcaatgtcttngcaagtccagacac

SEQ ID NO: 533 - DNA, 29 bp, Homo sapiens
  modified_base (15)..(15): a, c, g, t, unknown or other
ccagcaaatggaaantgaacaattttata

SEQ ID NO: 534 - DNA, 29 bp, Homo sapiens
  modified_base (15)..(15): a, c, g, t, unknown or other
aaagagccatttcantagaaaagatctgg

SEQ ID NO: 535 - DNA, 29 bp, Homo sapiens
  modified_base (15)..(15): a, c, g, t, unknown or other
ttaagtataggacangacctaaggacaat

SEQ ID NO: 536 - DNA, 29 bp, Homo sapiens
  modified_base (15)..(15): a, c, g, t, unknown or other
ttgtactaaacacantatgagcatcattt

SEQ ID NO: 537 - DNA, 29 bp, Homo sapiens
  modified_base (15)..(15): a, c, g, t, unknown or other
gtaagaaagagatangctactgttgtcgt

SEQ ID NO: 538 - DNA, 29 bp, Homo sapiens
  modified_base (15)..(15): a, c, g, t, unknown or other
gatcttccacaagcnaggtaacatcatca

SEQ ID NO: 539 - DNA, 29 bp, Homo sapiens
  modified_base (15)..(15): a, c, g, t, unknown or other
tgcaatttatactantaacaaaagtgaac

SEQ ID NO: 540 - DNA, 29 bp, Homo sapiens
  modified_base (15)..(15): a, c, g, t, unknown or other
aattagtaaaatctntgggagtttattct

SEQ ID NO: 541 - DNA, 29 bp, Homo sapiens
  modified_base (15)..(15): a, c, g, t, unknown or other
caggtattttgtatntatgagactagagg

SEQ ID NO: 542 - DNA, 29 bp, Homo sapiens
  modified_base (15)..(15): a, c, g, t, unknown or other
attactttagagctntacttttaaatact

SEQ ID NO: 543 - DNA, 29 bp, Homo sapiens
  modified_base (15)..(15): a, c, g, t, unknown or other
cctaaatgtcattcnggattgtactgtat

SEQ ID NO: 544 - DNA, 29 bp, Homo sapiens
  modified_base (15)..(15): a, c, g, t, unknown or other
actgacaacattagngtagatatttcaac

SEQ ID NO: 545 - DNA, 29 bp, Homo sapiens
  modified_base (15)..(15): a, c, g, t, unknown or other
ctgtatatgtattanaaatgttgggcata

SEQ ID NO: 546 - DNA, 29 bp, Homo sapiens
  modified_base (15)..(15): a, c, g, t, unknown or other
ctaaaactctggcantctgggcagttgat

SEQ ID NO: 547 - DNA, 29 bp, Homo sapiens
  modified_base (15)..(15): a, c, g, t, unknown or other
cagaaccatctcagntaagtctgatgaaa

SEQ ID NO: 548 - DNA, 29 bp, Homo sapiens
  modified_base (15)..(15): a, c, g, t, unknown or other
ctacatggaagtagnaccatgctcccagg

SEQ ID NO: 549 - DNA, 29 bp, Homo sapiens
  modified_base (15)..(15): a, c, g, t, unknown or other
taacaattgaaatcngtgaccttgagcag

SEQ ID NO: 550 - DNA, 29 bp, Homo sapiens
  modified_base (15)..(15): a, c, g, t, unknown or other
caacacatacagcantttagttaccttga

SEQ ID NO: 551 - DNA, 29 bp, Homo sapiens
  modified_base (15)..(15): a, c, g, t, unknown or other
atcatttttaccttnagtgcttaggctgg

SEQ ID NO: 552 - DNA, 29 bp, Homo sapiens
  modified_base (15)..(15): a, c, g, t, unknown or other
tccaacctagaaaanggcgatggtgttgg

SEQ ID NO: 553 - DNA, 29 bp, Homo sapiens
  modified_base (15)..(15): a, c, g, t, unknown or other
attgaagaagggacngcttcttgccaaat

SEQ ID NO: 554 - DNA, 29 bp, Homo sapiens
  modified_base (15)..(15): a, c, g, t, unknown or other
gtataatgtcaaaantgttgaatttaaat

SEQ ID NO: 555 - DNA, 29 bp, Homo sapiens
  modified_base (15)..(15): a, c, g, t, unknown or other
actttgtctaaatgnaagagttcatacac

SEQ ID NO: 556 - DNA, 29 bp, Homo sapiens
  modified_base (15)..(15): a, c, g, t, unknown or other
attccatggtgttcngaaaatattagggt

SEQ ID NO: 557 - DNA, 29 bp, Homo sapiens
  modified_base (15)..(15): a, c, g, t, unknown or other
acacaaaacagtagnagattccattggaa

SEQ ID NO: 558 - DNA, 29 bp, Homo sapiens
  modified_base (15)..(15): a, c, g, t, unknown or other
tcaaacaaggtctcnttaacacattgcat

SEQ ID NO: 559 - DNA, 29 bp, Homo sapiens
  modified_base (15)..(15): a, c, g, t, unknown or other
tgtgctcaatatcanattcttttttagat

SEQ ID NO: 560 - DNA, 29 bp, Homo sapiens
  modified_base (15)..(15): a, c, g, t, unknown or other
ttcattatgctacanggactggcttccat

SEQ ID NO: 561 - DNA, 29 bp, Homo sapiens
  modified_base (15)..(15): a, c, g, t, unknown or other
ggtctcataattagntttaaaatggggaa

SEQ ID NO: 562 - DNA, 29 bp, Homo sapiens
  modified_base (15)..(15): a, c, g, t, unknown or other
taacaatggagatgntatggctgatatag

SEQ ID NO: 563 - DNA, 29 bp, Homo sapiens
  modified_base (15)..(15): a, c, g, t, unknown or other
catcatcacagtgangcttgtgcaacagc

SEQ ID NO: 564 - DNA, 29 bp, Homo sapiens
  modified_base (15)..(15): a, c, g, t, unknown or other
atgttctgtggatcnctcaggtatctctt

SEQ ID NO: 565 - DNA, 29 bp, Homo sapiens
  modified_base (15)..(15): a, c, g, t, unknown or other
aaagagtcattcacngtggctttcattgg

SEQ ID NO: 566 - DNA, 29 bp, Homo sapiens
  modified_base (15)..(15): a, c, g, t, unknown or other
catcttgcatatctntgtgatgtgaataa

SEQ ID NO: 567 - DNA, 29 bp, Homo sapiens
  modified_base (15)..(15): a, c, g, t, unknown or other
ctctgcccagctagnaagtagcagaatca

SEQ ID NO: 568 - DNA, 29 bp, Homo sapiens
  modified_base (15)..(15): a, c, g, t, unknown or other
tttacatttactgcngcagatatctctgt

SEQ ID NO: 569 - DNA, 29 bp, Homo sapiens
  modified_base (15)..(15): a, c, g, t, unknown or other
ctgactgaccagggntggacagttaactc

SEQ ID NO: 570 - DNA, 29 bp, Homo sapiens
  modified_base (15)..(15): a, c, g, t, unknown or other
acactgaataccttntgatggcactgcca

SEQ ID NO: 571 - DNA, 29 bp, Homo sapiens
  modified_base (15)..(15): a, c, g, t, unknown or other
tcctgttggaatggngaatctcatattag

SEQ ID NO: 572 - DNA, 29 bp, Homo sapiens
  modified_base (15)..(15): a, c, g, t, unknown or other
aacaaaagtgtacgncaaaacccatataa

SEQ ID NO: 573 - DNA, 29 bp, Homo sapiens
  modified_base (15)..(15): a, c, g, t, unknown or other
gcttcaagttggaanatagcctacagttt

SEQ ID NO: 574 - DNA, 29 bp, Homo sapiens
  modified_base (15)..(15): a, c, g, t, unknown or other
aataatgtcaagaanatgctggcacataa

SEQ ID NO: 575 - DNA, 29 bp, Homo sapiens
  modified_base (15)..(15): a, c, g, t, unknown or other
aaaagcaagagtttnccaaagtgtaacag

SEQ ID NO: 576 - DNA, 29 bp, Homo sapiens
  modified_base (15)..(15): a, c, g, t, unknown or other
gcagtatggactggngcttacatcatgtc

SEQ ID NO: 577 - DNA, 29 bp, Homo sapiens
  modified_base (15)..(15): a, c, g, t, unknown or other
cacatcatgagtgtncccagttcattgtg

SEQ ID NO: 578 - DNA, 29 bp, Homo sapiens
  modified_base (15)..(15): a, c, g, t, unknown or other
acctgtcctctatgntatgacaaatatgc

SEQ ID NO: 579 - DNA, 29 bp, Homo sapiens
  modified_base (15)..(15): a, c, g, t, unknown or other
aataaaccattttcntcagtctaattatt

SEQ ID NO: 580 - DNA, 29 bp, Homo sapiens
  modified_base (15)..(15): a, c, g, t, unknown or other
tgcaaaggaaccaanaagcatgaggtaag

SEQ ID NO: 581 - DNA, 29 bp, Homo sapiens
  modified_base (15)..(15): a, c, g, t, unknown or other
ctgatgaaacctttnaaatccaatggatt

SEQ ID NO: 582 - DNA, 29 bp, Homo sapiens
  modified_base (15)..(15): a, c, g, t, unknown or other
aaataaaatagagtncaatgatatgccca

SEQ ID NO: 583 - DNA, 29 bp, Homo sapiens
  modified_base (15)..(15): a, c, g, t, unknown or other
ggctaaatcatttanaaacttgttaacac

SEQ ID NO: 584 - DNA, 29 bp, Homo sapiens
  modified_base (15)..(15): a, c, g, t, unknown or other
attttactgccctcngtgagctcaacatc

SEQ ID NO: 585 - DNA, 29 bp, Homo sapiens
  modified_base (15)..(15): a, c, g, t, unknown or other
tgacttaattcatcnggccatggagaagg

SEQ ID NO: 586 - DNA, 29 bp, Homo sapiens
  modified_base (15)..(15): a, c, g, t, unknown or other
ttagggagaaaaatnaataagttaataaa

SEQ ID NO: 587 - DNA, 29 bp, Homo sapiens
  modified_base (15)..(15): a, c, g, t, unknown or other
tgtttcatgttttanagaggacttaacac

SEQ ID NO: 588 - DNA, 29 bp, Homo sapiens
  modified_base (15)..(15): a, c, g, t, unknown or other
gtttgtactaaccangtgctgcttctaaa

SEQ ID NO: 589 - DNA, 29 bp, Homo sapiens
  modified_base (15)..(15): a, c, g, t, unknown or other
tgagagttgggggtncaagtatctgagta

SEQ ID NO: 590 - DNA, 29 bp, Homo sapiens
  modified_base (15)..(15): a, c, g, t, unknown or other
ccaaccctcaagggncattggtttctgaa

SEQ ID NO: 591 - DNA, 29 bp, Homo sapiens
  modified_base (15)..(15): a, c, g, t, unknown or other
gacccaaaaattacntttgtcaacccaaa

SEQ ID NO: 592 - DNA, 29 bp, Homo sapiens
  modified_base (15)..(15): a, c, g, t, unknown or other
aaatccaccataagnttcaaagattttgg

SEQ ID NO: 593 - DNA, 29 bp, Homo sapiens
  modified_base (15)..(15): a, c, g, t, unknown or other
taggtatatggtganggacatagatctcc

SEQ ID NO: 594 - DNA, 29 bp, Homo sapiens
  modified_base (15)..(15): a, c, g, t, unknown or other
tctaccaaatcctgntattctcacatggg

SEQ ID NO: 595 - DNA, 29 bp, Homo sapiens
  modified_base (15)..(15): a, c, g, t, unknown or other
tacagtgaaaaggcntagaagatcttgat

SEQ ID NO: 596 - DNA, 29 bp, Homo sapiens
  modified_base (15)..(15): a, c, g, t, unknown or other
cccagtcaagatgcntattctccccactt

SEQ ID NO: 597 - DNA, 29 bp, Homo sapiens
  modified_base (15)..(15): a, c, g, t, unknown or other
tgtttttctttccangagtacatcataca

SEQ ID NO: 598 - DNA, 29 bp, Homo sapiens
  modified_base (15)..(15): a, c, g, t, unknown or other
agcgggattaagtcnaggaaatgaacaaa

SEQ ID NO: 599 - DNA, 29 bp, Homo sapiens
  modified_base (15)..(15): a, c, g, t, unknown or other
agatgagatttgaangatgagtagagacc

SEQ ID NO: 600 - DNA, 29 bp, Homo sapiens
  modified_base (15)..(15): a, c, g, t, unknown or other
attataccacatatngtaatctctgcaga

SEQ ID NO: 601 - DNA, 29 bp, Homo sapiens
  modified_base (15)..(15): a, c, g, t, unknown or other
gaatacggatgttgnttattgtttgaata

SEQ ID NO: 602 - DNA, 29 bp, Homo sapiens
  modified_base (15)..(15): a, c, g, t, unknown or other
cattaattgtgtccnattttttttttctg

SEQ ID NO: 603 - DNA, 29 bp, Homo sapiens
  modified_base (15)..(15): a, c, g, t, unknown or other
ttgttgcttttgaanatcaattgactgct

SEQ ID NO: 604 - DNA, 29 bp, Homo sapiens
  modified_base (15)..(15): a, c, g, t, unknown or other
tgacaagcaaaaatngaatgatgactgca

SEQ ID NO: 605 - DNA, 29 bp, Homo sapiens
  modified_base (15)..(15): a, c, g, t, unknown or other
ttaagggttttgggnacccagctgcaatt

SEQ ID NO: 606 - DNA, 29 bp, Homo sapiens
  modified_base (15)..(15): a, c, g, t, unknown or other
taagattaaaatatntagccaccctcctt

SEQ ID NO: 607 - DNA, 29 bp, Homo sapiens
  modified_base (15)..(15): a, c, g, t, unknown or other
gacacagttctgacnaggttaaataagta

SEQ ID NO: 608 - DNA, 29 bp, Homo sapiens
  modified_base (15)..(15): a, c, g, t, unknown or other
atatcgatcaaaaangtcatgttgttcca

SEQ ID NO: 609 - DNA, 29 bp, Homo sapiens
  modified_base (15)..(15): a, c, g, t, unknown or other
tcttggatttagtanaggtctttcagatt

SEQ ID NO: 610 - DNA, 29 bp, Homo sapiens
  modified_base (15)..(15): a, c, g, t, unknown or other
taaccctcttccagnttcccacagagatc

SEQ ID NO: 611 - DNA, 29 bp, Homo sapiens
  modified_base (15)..(15): a, c, g, t, unknown or other
tccacctggaaaacnctcttgccaaatag

SEQ ID NO: 612 - DNA, 29 bp, Homo sapiens
  modified_base (15)..(15): a, c, g, t, unknown or other
agaacatttctccangtttctcaggtatg

SEQ ID NO: 613 - DNA, 29 bp, Homo sapiens
  modified_base (15)..(15): a, c, g, t, unknown or other
ccgatataattcttnaaaaatatgctggc

SEQ ID NO: 614 - DNA, 29 bp, Homo sapiens
  modified_base (15)..(15): a, c, g, t, unknown or other
attacattctgaaantgaaatgtaaaaga

SEQ ID NO: 615 - DNA, 29 bp, Homo sapiens
  modified_base (15)..(15): a, c, g, t, unknown or other
aatgtaagagaaacntctaccaaatgctg

SEQ ID NO: 616 - DNA, 29 bp, Homo sapiens
  modified_base (15)..(15): a, c, g, t, unknown or other
atgaacaaaagccantaattcccttcata

SEQ ID NO: 617 - DNA, 29 bp, Homo sapiens
  modified_base (15)..(15): a, c, g, t, unknown or other
ctccaatgttgtggnaagtgtgatagctg

SEQ ID NO: 618 - DNA, 29 bp, Homo sapiens
  modified_base (15)..(15): a, c, g, t, unknown or other
ttgactctaaaaagncacatcattcccca

SEQ ID NO: 619 - DNA, 29 bp, Homo sapiens
  modified_base (15)..(15): a, c, g, t, unknown or other
ctcttgcagtcagcngtcccagaaagaca

SEQ ID NO: 620 - DNA, 29 bp, Homo sapiens
  modified_base (15)..(15): a, c, g, t, unknown or other
ttctccttctgggcntatgagagtattat

SEQ ID NO: 621 - DNA, 29 bp, Homo sapiens
  modified_base (15)..(15): a, c, g, t, unknown or other
tgctgaatgtgtgcngtaactgcagtgtt

SEQ ID NO: 622 - DNA, 29 bp, Homo sapiens
  modified_base (15)..(15): a, c, g, t, unknown or other
ctttctgtctctgantaacaacaggttgc

SEQ ID NO: 623 - DNA, 29 bp, Homo sapiens
  modified_base (15)..(15): a, c, g, t, unknown or other
ctcttgagcacttangctttatttgtgta

SEQ ID NO: 624 - DNA, 29 bp, Homo sapiens
  modified_base (15)..(15): a, c, g, t, unknown or other
cctgtttttttttancttctaggaggttt

SEQ ID NO: 625 - DNA, 29 bp, Homo sapiens
  modified_base (15)..(15): a, c, g, t, unknown or other
tctcacagtattcancatgcaaattatgg

SEQ ID NO: 626 - DNA, 29 bp, Homo sapiens
  modified_base (15)..(15): a, c, g, t, unknown or other
tagccagcaaatacngaattacagaaatc

SEQ ID NO: 627 - DNA, 29 bp, Homo sapiens
  modified_base (15)..(15): a, c, g, t, unknown or other
agcacaatttttccnttttcacatttcag

SEQ ID NO: 628 - DNA, 29 bp, Homo sapiens
  modified_base (15)..(15): a, c, g, t, unknown or other
tctctctgttctacnatctttaaacatct

SEQ ID NO: 629 - DNA, 29 bp, Homo sapiens
  modified_base (15)..(15): a, c, g, t, unknown or other
actttagcaactccntgccgatctccata

SEQ ID NO: 630 - DNA, 29 bp, Homo sapiens
  modified_base (15)..(15): a, c, g, t, unknown or other
ctatagttctgtaangtgagcgccacctt

SEQ ID NO: 631 - DNA, 29 bp, Homo sapiens
  modified_base (15)..(15): a, c, g, t, unknown or other
cctatattctaaacnctaccttccaaaca

SEQ ID NO: 632 - DNA, 29 bp, Homo sapiens
  modified_base (15)..(15): a, c, g, t, unknown or other
tcccatgcttggtanagtacgtttactga

SEQ ID NO: 633 - DNA, 29 bp, Homo sapiens
  modified_base (15)..(15): a, c, g, t, unknown or other
aatgagacagatagnaatgttcaatagaa

SEQ ID NO: 634 - DNA, 29 bp, Homo sapiens
  modified_base (15)..(15): a, c, g, t, unknown or other
gtaagattttctgtntagtatcatcaaat

SEQ ID NO: 635 - DNA, 29 bp, Homo sapiens
  modified_base (15)..(15): a, c, g, t, unknown or other
aaccaccacgaatgnaggaaatcttcccc

SEQ ID NO: 636 - DNA, 29 bp, Homo sapiens
  modified_base (15)..(15): a, c, g, t, unknown or other
aattagagtagctcncagtatctgtctct

SEQ ID NO: 637 - DNA, 29 bp, Homo sapiens
  modified_base (15)..(15): a, c, g, t, unknown or other
tccaacactgattantgcagtcttaaaac

SEQ ID NO: 638 - DNA, 29 bp, Homo sapiens
  modified_base (15)..(15): a, c, g, t, unknown or other
ctgcaggcagggcanatctattaaaaaac

SEQ ID NO: 639 - DNA, 29 bp, Homo sapiens
  modified_base (15)..(15): a, c, g, t, unknown or other
ggcccattttagggngtgtgttttttctg

SEQ ID NO: 640 - DNA, 29 bp, Homo sapiens
  modified_base (15)..(15): a, c, g, t, unknown or other
aggatgggaactcanagtcatttaatttg

SEQ ID NO: 641 - DNA, 29 bp, Homo sapiens
  modified_base (15)..(15): a, c, g, t, unknown or other
cccagccagtcgagntgaccacgctgaaa

SEQ ID NO: 642 - DNA, 29 bp, Homo sapiens
  modified_base (15)..(15): a, c, g, t, unknown or other
tttctgaggttcacntctcatgtcatgat

SEQ ID NO: 643 - DNA, 29 bp, Homo sapiens
  modified_base (15)..(15): a, c, g, t, unknown or other
taagcagtgaattcngatattattcaggg

SEQ ID NO: 644 - DNA, 29 bp, Homo sapiens
  modified_base (15)..(15): a, c, g, t, unknown or other
gaacttgtacaatancctttacctctcca

SEQ ID NO: 645 - DNA, 29 bp, Homo sapiens
  modified_base (15)..(15): a, c, g, t, unknown or other
caggcttgacagttnactaaaccctcgca

SEQ ID NO: 646 - DNA, 29 bp, Homo sapiens
  modified_base (15)..(15): a, c, g, t, unknown or other
tgaggacttctcccnttaaaagttcaagt

SEQ ID NO: 647 - DNA, 29 bp, Homo sapiens
  modified_base (15)..(15): a, c, g, t, unknown or other
ggtcacaaaagaaanccgtgtttaagtca

SEQ ID NO: 648 - DNA, 29 bp, Homo sapiens
  modified_base (15)..(15): a, c, g, t, unknown or other
gtctatacacacaanttaatgaacactgc

SEQ ID NO: 649 - DNA, 29 bp, Homo sapiens
  modified_base (15)..(15): a, c, g, t, unknown or other
gaggagattccccangttcactttgaata

SEQ ID NO: 650 - DNA, 29 bp, Homo sapiens
  modified_base (15)..(15): a, c, g, t, unknown or other
actttaatttcgccntccccttccagcag

SEQ ID NO: 651 - DNA, 29 bp, Homo sapiens
  modified_base (15)..(15): a, c, g, t, unknown or other
gtcaagggacacagnagagctatttcagg

SEQ ID NO: 652 - DNA, 29 bp, Homo sapiens
  modified_base (15)..(15): a, c, g, t, unknown or other
ggtcaactgatgatntgctaataaatgaa

SEQ ID NO: 653 - DNA, 29 bp, Homo sapiens
  modified_base (15)..(15): a, c, g, t, unknown or other
taaacatactcaaanatttcagcagtctt

SEQ ID NO: 654 - DNA, 29 bp, Homo sapiens
  modified_base (15)..(15): a, c, g, t, unknown or other
ttccacaatgcgtgnaatatttcactaaa

SEQ ID NO: 655 - DNA, 29 bp, Homo sapiens
  modified_base (15)..(15): a, c, g, t, unknown or other
cactggcccctgganacgttgtcctcaca

SEQ ID NO: 656 - DNA, 29 bp, Homo sapiens
  modified_base (15)..(15): a, c, g, t, unknown or other
agcaggggcagcccnaggtctattcctat

SEQ ID NO: 657 - DNA, 29 bp, Homo sapiens
  modified_base (15)..(15): a, c, g, t, unknown or other
tctagtctagcccangatttcttgacctt

SEQ ID NO: 658 - DNA, 29 bp, Homo sapiens
  modified_base (15)..(15): a, c, g, t, unknown or other
aggcagtttctaacntaagaggtagttca

SEQ ID NO: 659 - DNA, 29 bp, Homo sapiens
  modified_base (15)..(15): a, c, g, t, unknown or other
cctttctaccaaaancttcctccttcagc

SEQ ID NO: 660 - DNA, 29 bp, Homo sapiens
  modified_base (15)..(15): a, c, g, t, unknown or other
tgatcactggatgancatgtcaagcaaga

SEQ ID NO: 661 - DNA, 29 bp, Homo sapiens
  modified_base (15)..(15): a, c, g, t, unknown or other
gctgggaaaatcagntgtatctttgattc

SEQ ID NO: 662 - DNA, 29 bp, Homo sapiens
  modified_base (15)..(15): a, c, g, t, unknown or other
ctgtgtggccagggngcagacagaagtga

SEQ ID NO: 663 - DNA, 29 bp, Homo sapiens
  modified_base (15)..(15): a, c, g, t, unknown or other
gtgtgctataaccantcagagagataaga

SEQ ID NO: 664 - DNA, 29 bp, Homo sapiens
  modified_base (15)..(15): a, c, g, t, unknown or other
ctcatttcccactanttaactgagagcaa

SEQ ID NO: 665 - DNA, 29 bp, Homo sapiens
  modified_base (15)..(15): a, c, g, t, unknown or other
tgaagatgacaaaantttctcaatgcagc

SEQ ID NO: 666 - DNA, 29 bp, Homo sapiens
  modified_base (15)..(15): a, c, g, t, unknown or other
ggaaggatgtagacngactagaaagacaa

SEQ ID NO: 667 - DNA, 29 bp, Homo sapiens
  modified_base (15)..(15): a, c, g, t, unknown or other
tctgtagaatgcagnatccctcaagatgg

SEQ ID NO: 668 - DNA, 29 bp, Homo sapiens
  modified_base (15)..(15): a, c, g, t, unknown or other
gtttttcttactctncctgaagtctcctg

SEQ ID NO: 669 - DNA, 29 bp, Homo sapiens
  modified_base (15)..(15): a, c, g, t, unknown or other
aactgaggtagatgntataaagaaaggga

SEQ ID NO: 670 - DNA, 29 bp, Homo sapiens
  modified_base (15)..(15): a, c, g, t, unknown or other
aggaatctggttttntgatggggagccaa

SEQ ID NO: 671 - DNA, 29 bp, Homo sapiens
  modified_base (15)..(15): a, c, g, t, unknown or other
ccttgacatgtctgngtaaaggcttaatc

SEQ ID NO: 672 - DNA, 29 bp, Homo sapiens
  modified_base (15)..(15): a, c, g, t, unknown or other
caaatcttcttcccntaggtccatggcaa

SEQ ID NO: 673 - DNA, 29 bp, Homo sapiens
  modified_base (15)..(15): a, c, g, t, unknown or other
tggggataaattgtncttcccttacaggg

SEQ ID NO: 674 - DNA, 29 bp, Homo sapiens
  modified_base (15)..(15): a, c, g, t, unknown or other
cttaatcacagctcnactgcttacaagct

SEQ ID NO: 675 - DNA, 29 bp, Homo sapiens
  modified_base (15)..(15): a, c, g, t, unknown or other
gagccgaacactcantgttatggaaacgg

SEQ ID NO: 676 - DNA, 29 bp, Homo sapiens
  modified_base (15)..(15): a, c, g, t, unknown or other
gggacaaatgatgcngaggaaagagaaaa

SEQ ID NO: 677 - DNA, 29 bp, Homo sapiens
  modified_base (15)..(15): a, c, g, t, unknown or other
cttaagataagaacntctcattgtaatta

SEQ ID NO: 678 - DNA, 29 bp, Homo sapiens
  modified_base (15)..(15): a, c, g, t, unknown or other
gctggggtcttgcangcttgcaacagcct

SEQ ID NO: 679 - DNA, 29 bp, Homo sapiens
  modified_base (15)..(15): a, c, g, t, unknown or other
attactcacacagcngatcaagttactcc

SEQ ID NO: 680 - DNA, 29 bp, Homo sapiens
  modified_base (15)..(15): a, c, g, t, unknown or other
tcaagtaacagacanggcatcgcctttgc

SEQ ID NO: 681 - DNA, 29 bp, Homo sapiens
  modified_base (15)..(15): a, c, g, t, unknown or other
aatccataaggtaantggttttctgcagg

SEQ ID NO: 682 - DNA, 29 bp, Homo sapiens
  modified_base (15)..(15): a, c, g, t, unknown or other
tgctttgggaggagngacctctgagctac

SEQ ID NO: 683 - DNA, 29 bp, Homo sapiens
  modified_base (15)..(15): a, c, g, t, unknown or other
aaattagggccttangtttcattaccaag

SEQ ID NO: 684 - DNA, 29 bp, Homo sapiens
  modified_base (15)..(15): a, c, g, t, unknown or other
gagactaatcaaggnacatgatgtgacgc

SEQ ID NO: 685 - DNA, 29 bp, Homo sapiens
  modified_base (15)..(15): a, c, g, t, unknown or other
agtaaattagatagnatgttggaaggccg

SEQ ID NO: 686 - DNA, 29 bp, Homo sapiens
  modified_base (15)..(15): a, c, g, t, unknown or other
ggccagacagagtgnccaaaacccctgtt

SEQ ID NO: 687 - DNA, 29 bp, Homo sapiens
  modified_base (15)..(15): a, c, g, t, unknown or other
actgtggagataganaagggagatggtca

SEQ ID NO: 688 - DNA, 29 bp, Homo sapiens
  modified_base (15)..(15): a, c, g, t, unknown or other
gaactatttttcccntttgttctaggtgg

SEQ ID NO: 689 - DNA, 29 bp, Homo sapiens
  modified_base (15)..(15): a, c, g, t, unknown or other
atactttcctgtatnttgggcttttggca

SEQ ID NO: 690 - DNA, 29 bp, Homo sapiens
  modified_base (15)..(15): a, c, g, t, unknown or other
tggttttcaagcaangtggttttcaattt

SEQ ID NO: 691 - DNA, 29 bp, Homo sapiens
  modified_base (15)..(15): a, c, g, t, unknown or other
caactgtaagataanctctgtgtgcatca

SEQ ID NO: 692 - DNA, 29 bp, Homo sapiens
  modified_base (15)..(15): a, c, g, t, unknown or other
atatgcttgctgtgnatgtctccccctgt

SEQ ID NO: 693 - DNA, 29 bp, Homo sapiens
  modified_base (15)..(15): a, c, g, t, unknown or other
actattaagaacaantgtgggatttattc

SEQ ID NO: 694 - DNA, 29 bp, Homo sapiens
  modified_base (15)..(15): a, c, g, t, unknown or other
tgcttagcagtggtngacgagtcacctgg

SEQ ID NO: 695 - DNA, 29 bp, Homo sapiens
  modified_base (15)..(15): a, c, g, t, unknown or other
taaaaatgtaggctnactgtcactagcca

SEQ ID NO: 696 - DNA, 29 bp, Homo sapiens
  modified_base (15)..(15): a, c, g, t, unknown or other
aaaacctttaaaaanatatctccagtgtt

SEQ ID NO: 697 - DNA, 29 bp, Homo sapiens
  modified_base (15)..(15): a, c, g, t, unknown or other
tggacaatgaaaacntagtcaattttaac

SEQ ID NO: 698 - DNA, 29 bp, Homo sapiens
  modified_base (15)..(15): a, c, g, t, unknown or other
ctgcagggagagccnccgtctgccgccct

SEQ ID NO: 699 - DNA, 29 bp, Homo sapiens
  modified_base (15)..(15): a, c, g, t, unknown or other
cctgtcagattcccngctcacagctcaaa

SEQ ID NO: 700 - DNA, 29 bp, Homo sapiens
  modified_base (15)..(15): a, c, g, t, unknown or other
atgtgtccaaaagcngtctgcaggtcaca

SEQ ID NO: 701 - DNA, 29 bp, Homo sapiens
  modified_base (15)..(15): a, c, g, t, unknown or other
ctgtgcactgatganttcagaagcagaag

SEQ ID NO: 702 - DNA, 29 bp, Homo sapiens
  modified_base (15)..(15): a, c, g, t, unknown or other
tctagtattcttganagcaactgctttaa

SEQ ID NO: 703 - DNA, 29 bp, Homo sapiens
  modified_base (15)..(15): a, c, g, t, unknown or other
caagacttagaagantaagcatggtagaa

SEQ ID NO: 704 - DNA, 29 bp, Homo sapiens
  modified_base (15)..(15): a, c, g, t, unknown or other
aaagggtgtttactngcaacatttttgaa

SEQ ID NO: 705 - DNA, 29 bp, Homo sapiens
  modified_base (15)..(15): a, c, g, t, unknown or other
ttagattcagcagcnttccagcgctctgc

SEQ ID NO: 706 - DNA, 29 bp, Homo sapiens
  modified_base (15)..(15): a, c, g, t, unknown or other
ttactgtgtgcaagngtagaatccaagtc

SEQ ID NO: 707 - DNA, 29 bp, Homo sapiens
  modified_base (15)..(15): a, c, g, t, unknown or other
gaagagtagaaaaanagtagatgaagatg

SEQ ID NO: 708 - DNA, 29 bp, Homo sapiens
  modified_base (15)..(15): a, c, g, t, unknown or other
tgtaaccatagattnacttgagtgataag

SEQ ID NO: 709 - DNA, 29 bp, Homo sapiens
  modified_base (15)..(15): a, c, g, t, unknown or other
aatgtgtcaaatganaggagggttaagtg

SEQ ID NO: 710 - DNA, 29 bp, Homo sapiens
  modified_base (15)..(15): a, c, g, t, unknown or other
aatgcagacacgccntgatgactgtggtg

SEQ ID NO: 711 - DNA, 29 bp, Homo sapiens
  modified_base (15)..(15): a, c, g, t, unknown or other
ggacaatcaacactncttcctggtttgaa

SEQ ID NO: 712 - DNA, 29 bp, Homo sapiens
  modified_base (15)..(15): a, c, g, t, unknown or other
agagcacgtgtgagnaagatgcccctctc

SEQ ID NO: 713 - DNA, 29 bp, Homo sapiens
  modified_base (15)..(15): a, c, g, t, unknown or other
actttgctgttaccnctcctatgactgtg

SEQ ID NO: 714 - DNA, 29 bp, Homo sapiens
  modified_base (15)..(15): a, c, g, t, unknown or other
agagcactcacaatnggcagagtcacatg

SEQ ID NO: 715 - DNA, 29 bp, Homo sapiens
  modified_base (15)..(15): a, c, g, t, unknown or other
ctaggatctgcagangtattccctggctg

SEQ ID NO: 716 - DNA, 29 bp, Homo sapiens
  modified_base (15)..(15): a, c, g, t, unknown or other
gtagggagatagctntaagggagaaagac

SEQ ID NO: 717 - DNA, 29 bp, Homo sapiens
  modified_base (15)..(15): a, c, g, t, unknown or other
agagaaacttcaagncctgatcttgactg

SEQ ID NO: 718 - DNA, 29 bp, Homo sapiens
  modified_base (15)..(15): a, c, g, t, unknown or other
ctatatccatttacncagtgaatactgag

SEQ ID NO: 719 - DNA, 29 bp, Homo sapiens
  modified_base (15)..(15): a, c, g, t, unknown or other
gctcaccaaatttangcaaggagcatgaa

SEQ ID NO: 720 - DNA, 29 bp, Homo sapiens
  modified_base (15)..(15): a, c, g, t, unknown or other
gtaacttgccaaagntttcaggatacaca

SEQ ID NO: 721 - DNA, 29 bp, Homo sapiens
  modified_base (15)..(15): a, c, g, t, unknown or other
ggatgttggccaacngagccagtttctct

SEQ ID NO: 722 - DNA, 29 bp, Homo sapiens
  modified_base (15)..(15): a, c, g, t, unknown or other
accctcactgaagangtttgcaagaaccc

SEQ ID NO: 723 - DNA, 29 bp, Homo sapiens
  modified_base (15)..(15): a, c, g, t, unknown or other
cacaggtaggaaacngccagatgcaaatt

SEQ ID NO: 724 - DNA, 29 bp, Homo sapiens
  modified_base (15)..(15): a, c, g, t, unknown or other
gaagttctaataccnacctgacacgtttc

SEQ ID NO: 725 - DNA, 29 bp, Homo sapiens
  modified_base (15)..(15): a, c, g, t, unknown or other
gcagtctctgatctncgggactaaaaaca

SEQ ID NO: 726 - DNA, 29 bp, Homo sapiens
  modified_base (15)..(15): a, c, g, t, unknown or other
ttccaatctttatanccattctttttcct

SEQ ID NO: 727 - DNA, 29 bp, Homo sapiens
  modified_base (15)..(15): a, c, g, t, unknown or other
gcttaatcttttttngcatcagagtttga

SEQ ID NO: 728 - DNA, 29 bp, Homo sapiens
  modified_base (15)..(15): a, c, g, t, unknown or other
gcatggaagagcagnaccttatgttaggc

SEQ ID NO: 729 - DNA, 29 bp, Homo sapiens
  modified_base (15)..(15): a, c, g, t, unknown or other
gaagagaccagaganatcaaagaagctcc

SEQ ID NO: 730 - DNA, 29 bp, Homo sapiens
  modified_base (15)..(15): a, c, g, t, unknown or other
cttgctgagtggtcngttctgagaaggca

SEQ ID NO: 731 - DNA, 29 bp, Homo sapiens
  modified_base (15)..(15): a, c, g, t, unknown or other
gtaacaattttacangtctacttttgaag

SEQ ID NO: 732 - DNA, 29 bp, Homo sapiens
  modified_base (15)..(15): a, c, g, t, unknown or other
ggactcctttattangagagaagccatca

SEQ ID NO: 733 - DNA, 29 bp, Homo sapiens
  modified_base (15)..(15): a, c, g, t, unknown or other
gtgttttgcctttcntaatagccagatac

SEQ ID NO: 734 - DNA, 29 bp, Homo sapiens
  modified_base (15)..(15): a, c, g, t, unknown or other
ccagtaatactggcnattaatgtattcaa

SEQ ID NO: 735 - DNA, 29 bp, Homo sapiens
  modified_base (15)..(15): a, c, g, t, unknown or other
tcttattcaatttgnatttgtgttacata

SEQ ID NO: 736 - DNA, 29 bp, Homo sapiens
  modified_base (15)..(15): a, c, g, t, unknown or other
aacaataaagttccnaattgaaagatgta

SEQ ID NO: 737 - DNA, 29 bp, Homo sapiens
  modified_base (15)..(15): a, c, g, t, unknown or other
aatgcatacattaanagatatagattcac

SEQ ID NO: 738 - DNA, 29 bp, Homo sapiens
  modified_base (15)..(15): a, c, g, t, unknown or other
tagaatagattacantataccaaaggaaa

SEQ ID NO: 739 - DNA, 29 bp, Homo sapiens
  modified_base (15)..(15): a, c, g, t, unknown or other
atcctctctttaggnatccacagtttatg

SEQ ID NO: 740 - DNA, 29 bp, Homo sapiens
  modified_base (15)..(15): a, c, g, t, unknown or other
tgagggaagccttcnaaaagtcacctaca

SEQ ID NO: 741 - DNA, 29 bp, Homo sapiens
  modified_base (15)..(15): a, c, g, t, unknown or other
atgagctttgtatanttgtaatcttttat

SEQ ID NO: 742 - DNA, 29 bp, Homo sapiens
  modified_base (15)..(15): a, c, g, t, unknown or other
cattaatcagtttcngaaattaagagata

SEQ ID NO: 743 - DNA, 29 bp, Homo sapiens
  modified_base (15)..(15): a, c, g, t, unknown or other
tacagttttaggacnaaattgcaaaatga

SEQ ID NO: 744 - DNA, 29 bp, Homo sapiens
  modified_base (15)..(15): a, c, g, t, unknown or other
taatattctccactngggtattatgcaaa

SEQ ID NO: 745 - DNA, 29 bp, Homo sapiens
  modified_base (15)..(15): a, c, g, t, unknown or other
tctgccaaccggttnacctctcgctttca

SEQ ID NO: 746 - DNA, 29 bp, Homo sapiens
  modified_base (15)..(15): a, c, g, t, unknown or other
tatttgccaaaatanaagaaacttgtatc

SEQ ID NO: 747 - DNA, 29 bp, Homo sapiens
  modified_base (15)..(15): a, c, g, t, unknown or other
tacttttaactcccncaaaacttgttttc

SEQ ID NO: 748 - DNA, 29 bp, Homo sapiens
  modified_base (15)..(15): a, c, g, t, unknown or other
tttatttcatattgnaaaatgtatgcatg

SEQ ID NO: 749 - DNA, 29 bp, Homo sapiens
  modified_base (15)..(15): a, c, g, t, unknown or other
atctttgctttgccngtggatagtttatg

SEQ ID NO: 750 - DNA, 29 bp, Homo sapiens
  modified_base (15)..(15): a, c, g, t, unknown or other
cctccgccctgtctnttcaccatagtttg

SEQ ID NO: 751 - DNA, 29 bp, Homo sapiens
  modified_base (15)..(15): a, c, g, t, unknown or other
gctacttccatcacntacagatccaagca

SEQ ID NO: 752 - DNA, 29 bp, Homo sapiens
  modified_base (15)..(15): a, c, g, t, unknown or other
acaaattatgctttngtgaatagtcctag

SEQ ID NO: 753 - DNA, 29 bp, Homo sapiens
  modified_base (15)..(15): a, c, g, t, unknown or other
tggaatcctccccantatctgacccggtt

SEQ ID NO: 754 - DNA, 29 bp, Homo sapiens
  modified_base (15)..(15): a, c, g, t, unknown or other
aatggaaatcatganggaccgggactggg

SEQ ID NO: 755 - DNA, 29 bp, Homo sapiens
  modified_base (15)..(15): a, c, g, t, unknown or other
tgcagttaacttggnattgttatgtaact

SEQ ID NO: 756 - DNA, 29 bp, Homo sapiens
  modified_base (15)..(15): a, c, g, t, unknown or other
tatccttccagtttncgaagagccagaaa

SEQ ID NO: 757 - DNA, 29 bp, Homo sapiens
  modified_base (15)..(15): a, c, g, t, unknown or other
cattgagcaaaacgnccatggtctttctt

SEQ ID NO: 758 - DNA, 29 bp, Homo sapiens
  modified_base (15)..(15): a, c, g, t, unknown or other
aaagtcagattaganagatatgatatgaa

SEQ ID NO: 759 - DNA, 29 bp, Homo sapiens
  modified_base (15)..(15): a, c, g, t, unknown or other
agcagtttataaaanaaagaacttgttgc

SEQ ID NO: 760 - DNA, 29 bp, Homo sapiens
  modified_base (15)..(15): a, c, g, t, unknown or other
ttcaggatagaagantcatggcagagggc

SEQ ID NO: 761 - DNA, 29 bp, Homo sapiens
  modified_base (15)..(15): a, c, g, t, unknown or other
taaagttttcactanaggtctgcatttat

SEQ ID NO: 762 - DNA, 29 bp, Homo sapiens
  modified_base (15)..(15): a, c, g, t, unknown or other
ctcagctggaaaganctgtttttgctgga

SEQ ID NO: 763 - DNA, 29 bp, Homo sapiens
  modified_base (15)..(15): a, c, g, t, unknown or other
tggttttggccttantatacagtagtttc

SEQ ID NO: 764 - DNA, 29 bp, Homo sapiens
  modified_base (15)..(15): a, c, g, t, unknown or other
ttgcccagggtctcnttgtacttttagca

SEQ ID NO: 765 - DNA, 29 bp, Homo sapiens
  modified_base (15)..(15): a, c, g, t, unknown or other
aagcttattgaggtntaatttgtatacag

SEQ ID NO: 766 - DNA, 29 bp, Homo sapiens
  modified_base (15)..(15): a, c, g, t, unknown or other
acacttaccaaaatntccgccaacgatca

SEQ ID NO: 767 - DNA, 29 bp, Homo sapiens
  modified_base (15)..(15): a, c, g, t, unknown or other
ggtgaagaataaacnaaaagcgttctggg

SEQ ID NO: 768 - DNA, 29 bp, Homo sapiens
  modified_base (15)..(15): a, c, g, t, unknown or other
atccacacaactaangtcagttccactaa

SEQ ID NO: 769 - DNA, 29 bp, Homo sapiens
  modified_base (15)..(15): a, c, g, t, unknown or other
gaattaaattacaantgcaatcagtcatg

SEQ ID NO: 770 - DNA, 29 bp, Homo sapiens
  modified_base (15)..(15): a, c, g, t, unknown or other
tgatcactgaaatantaaagtgatactaa

SEQ ID NO: 771 - DNA, 29 bp, Homo sapiens
  modified_base (15)..(15): a, c, g, t, unknown or other
gccaacaggtatcanttacttatacaaca

SEQ ID NO: 772 - DNA, 29 bp, Homo sapiens
  modified_base (15)..(15): a, c, g, t, unknown or other
ataatactttcaggnccaacaatactggc

SEQ ID NO: 773 - DNA, 29 bp, Homo sapiens
  modified_base (15)..(15): a, c, g, t, unknown or other
cttctaaatagacangacatttgtgtgga

SEQ ID NO: 774 - DNA, 29 bp, Homo sapiens
  modified_base (15)..(15): a, c, g, t, unknown or other
tggagttctagtggnatgttgggtagaag

SEQ ID NO: 775 - DNA, 29 bp, Homo sapiens
  modified_base (15)..(15): a, c, g, t, unknown or other
attgcttactccctntaggcagcagaatt

SEQ ID NO: 776 - DNA, 29 bp, Homo sapiens
  modified_base (15)..(15): a, c, g, t, unknown or other
cgccattcttagttnaaattgcccttcag

SEQ ID NO: 777 - DNA, 29 bp, Homo sapiens
  modified_base (15)..(15): a, c, g, t, unknown or other
tacttgattttttcnttttagatagttag

SEQ ID NO: 778 - DNA, 29 bp, Homo sapiens
  modified_base (15)..(15): a, c, g, t, unknown or other
cacacaagcactatngtgacgcgcgcttt

SEQ ID NO: 779 - DNA, 29 bp, Homo sapiens
  modified_base (15)..(15): a, c, g, t, unknown or other
aggggtgagtccccnggtcagggcagtaa

SEQ ID NO: 780 - DNA, 29 bp, Homo sapiens
  modified_base (15)..(15): a, c, g, t, unknown or other
gcaaataattatggngtgcccactctgca

SEQ ID NO: 781 - DNA, 29 bp, Homo sapiens
  modified_base (15)..(15): a, c, g, t, unknown or other
ttcttttgatgatangattgttcaggtca

SEQ ID NO: 782 - DNA, 29 bp, Homo sapiens
  modified_base (15)..(15): a, c, g, t, unknown or other
tttgattattgtcantggagctttgggct

SEQ ID NO: 783 - DNA, 29 bp, Homo sapiens
  modified_base (15)..(15): a, c, g, t, unknown or other
acatttcagtaccantggaaaatctctcc

SEQ ID NO: 784 - DNA, 29 bp, Homo sapiens
  modified_base (15)..(15): a, c, g, t, unknown or other
ttacagttgaggttnattacttctggttc

SEQ ID NO: 785 - DNA, 29 bp, Homo sapiens
  modified_base (15)..(15): a, c, g, t, unknown or other
aaagacatgtagttnaggtataaagatgc

SEQ ID NO: 786 - DNA, 29 bp, Homo sapiens
  modified_base (15)..(15): a, c, g, t, unknown or other
tctcagagcacatgnatatatgaactgga

SEQ ID NO: 787 - DNA, 29 bp, Homo sapiens
  modified_base (15)..(15): a, c, g, t, unknown or other
atagctctttttgtntacggtgaaattac

SEQ ID NO: 788 - DNA, 29 bp, Homo sapiens
  modified_base (15)..(15): a, c, g, t, unknown or other
tcttcatttcagtanccttctaaaagtct

SEQ ID NO: 789 - DNA, 29 bp, Homo sapiens
  modified_base (15)..(15): a, c, g, t, unknown or other
tcaccaagtcaattnaatacttttaattt

SEQ ID NO: 790 - DNA, 29 bp, Homo sapiens
  modified_base (15)..(15): a, c, g, t, unknown or other
attcctgagggggantcttgatgatgaag

SEQ ID NO: 791 - DNA, 29 bp, Homo sapiens
  modified_base (15)..(15): a, c, g, t, unknown or other
ttttcaattatcccnaaataaacaatgtc

SEQ ID NO: 792 - DNA, 29 bp, Homo sapiens
  modified_base (15)..(15): a, c, g, t, unknown or other
accctacccacttangtgcttataaagtc

SEQ ID NO: 793 - DNA, 29 bp, Homo sapiens
  modified_base (15)..(15): a, c, g, t, unknown or other
aaatcaggagcattnaagcttgtaaatct

SEQ ID NO: 794 - DNA, 29 bp, Homo sapiens
  modified_base (15)..(15): a, c, g, t, unknown or other
tacattcctatcacnagagggttaaatag

SEQ ID NO: 795 - DNA, 29 bp, Homo sapiens
  modified_base (15)..(15): a, c, g, t, unknown or other
taactagactttcanctggagttcatatg

SEQ ID NO: 796 - DNA, 29 bp, Homo sapiens
  modified_base (15)..(15): a, c, g, t, unknown or other
atgtttaaagtgtantaaggcaaaaacaa

SEQ ID NO: 797 - DNA, 29 bp, Homo sapiens
  modified_base (15)..(15): a, c, g, t, unknown or other
tgctctcaactgacntactcctacattgc

SEQ ID NO: 798 - DNA, 29 bp, Homo sapiens
  modified_base (15)..(15): a, c, g, t, unknown or other
atttttaaatgtacngtagaaggaaagaa

SEQ ID NO: 799 - DNA, 29 bp, Homo sapiens
  modified_base (15)..(15): a, c, g, t, unknown or other
aagtggaggtacacnagcaactgcagtgt

SEQ ID NO: 800 - DNA, 29 bp, Homo sapiens
  modified_base (15)..(15): a, c, g, t, unknown or other
gatcccaaactgccnaggtcaagggccca

SEQ ID NO: 801 - DNA, 29 bp, Homo sapiens
  modified_base (15)..(15): a, c, g, t, unknown or other
ttacttaaagacacntttctgggagaaat

SEQ ID NO: 802 - DNA, 29 bp, Homo sapiens
  modified_base (15)..(15): a, c, g, t, unknown or other
actatgactacacantattactttttctt

SEQ ID NO: 803 - DNA, 29 bp, Homo sapiens
  modified_base (15)..(15): a, c, g, t, unknown or other
tagtctgtaagatgntttagtaattaagg

SEQ ID NO: 804 - DNA, 29 bp, Homo sapiens
  modified_base (15)..(15): a, c, g, t, unknown or other
agaaacacaatcatnttgcttctaccata

SEQ ID NO: 805 - DNA, 29 bp, Homo sapiens
  modified_base (15)..(15): a, c, g, t, unknown or other
actaccatttgtacntgtgtgggcgtaaa

SEQ ID NO: 806 - DNA, 29 bp, Homo sapiens
  modified_base (15)..(15): a, c, g, t, unknown or other
gagcacttagtaacngatgaaagagggct

SEQ ID NO: 807 - DNA, 29 bp, Homo sapiens
  modified_base (15)..(15): a, c, g, t, unknown or other
gttattatgccaccnccctacccaatctc

SEQ ID NO: 808 - DNA, 29 bp, Homo sapiens
  modified_base (15)..(15): a, c, g, t, unknown or other
agatgagatttcctncattcctaacccat

SEQ ID NO: 809 - DNA, 29 bp, Homo sapiens
  modified_base (15)..(15): a, c, g, t, unknown or other
ccatctgtttttacnaatattggtttttt

SEQ ID NO: 810 - DNA, 29 bp, Homo sapiens
  modified_base (15)..(15): a, c, g, t, unknown or other
tggagatgggcgagnttaacaacttctca

SEQ ID NO: 811 - DNA, 29 bp, Homo sapiens
  modified_base (15)..(15): a, c, g, t, unknown or other
aaatctgtgcaatangtaaatctacctta

SEQ ID NO: 812 - DNA, 29 bp, Homo sapiens
  modified_base (15)..(15): a, c, g, t, unknown or other
ggtgtagctcaaaantttttatagctcta

SEQ ID NO: 813 - DNA, 29 bp, Homo sapiens
  modified_base (15)..(15): a, c, g, t, unknown or other
acaattgttaccaangttaaactctctac

SEQ ID NO: 814 - DNA, 29 bp, Homo sapiens
  modified_base (15)..(15): a, c, g, t, unknown or other
tttcaagtaaactangctgtgtatggtac

SEQ ID NO: 815 - DNA, 29 bp, Homo sapiens
  modified_base (15)..(15): a, c, g, t, unknown or other
acatctgagagggancctattagaccaca

SEQ ID NO: 816 - DNA, 29 bp, Homo sapiens
  modified_base (15)..(15): a, c, g, t, unknown or other
ctcaagcagtttatntgccagaattataa

SEQ ID NO: 817 - DNA, 29 bp, Homo sapiens
  modified_base (15)..(15): a, c, g, t, unknown or other
cctctaatgtataanggttccaaatctac

SEQ ID NO: 818 - DNA, 29 bp, Homo sapiens
  modified_base (15)..(15): a, c, g, t, unknown or other
aaattgaatttcagncagtaagcattgtt

SEQ ID NO: 819 - DNA, 29 bp, Homo sapiens
  modified_base (15)..(15): a, c, g, t, unknown or other
tcttctttttatccnttcccatctcagat

SEQ ID NO: 820 - DNA, 29 bp, Homo sapiens
  modified_base (15)..(15): a, c, g, t, unknown or other
aactgccctaaaagntacaaagcaaatgt

SEQ ID NO: 821 - DNA, 29 bp, Homo sapiens
  modified_base (15)..(15): a, c, g, t, unknown or other
gctgtagcatttaancaactatgcacaga

SEQ ID NO: 822 - DNA, 29 bp, Homo sapiens
  modified_base (15)..(15): a, c, g, t, unknown or other
gtggattttgttaanaagaggttagaaaa

SEQ ID NO: 823 - DNA, 29 bp, Homo sapiens
  modified_base (15)..(15): a, c, g, t, unknown or other
aataaggtcaaactnttgataactagaat

SEQ ID NO: 824 - DNA, 29 bp, Homo sapiens
  modified_base (15)..(15): a, c, g, t, unknown or other
cctcttaggtatgantgcatgcctgaagc

SEQ ID NO: 825 - DNA, 29 bp, Homo sapiens
  modified_base (15)..(15): a, c, g, t, unknown or other
gtgtgatctaggtgncaatgaactttgcc

SEQ ID NO: 826 - DNA, 29 bp, Homo sapiens
  modified_base (15)..(15): a, c, g, t, unknown or other
tattttaactcttgnccccattatcatat

SEQ ID NO: 827 - DNA, 29 bp, Homo sapiens
  modified_base (15)..(15): a, c, g, t, unknown or other
atcttcaaatactanccttgactttttct

SEQ ID NO: 828 - DNA, 29 bp, Homo sapiens
  modified_base (15)..(15): a, c, g, t, unknown or other
ctgtcaaaagtaacntcagaacacaaaag

SEQ ID NO: 829 - DNA, 29 bp, Homo sapiens
  modified_base (15)..(15): a, c, g, t, unknown or other
aagtgtaatgttttnttccaattgtgaga

SEQ ID NO: 830 - DNA, 29 bp, Homo sapiens
  modified_base (15)..(15): a, c, g, t, unknown or other
aatgccaccatgatnaatgatgccatttt

SEQ ID NO: 831 - DNA, 29 bp, Homo sapiens
  modified_base (15)..(15): a, c, g, t, unknown or other
ataattctgagcttnacatttttcatact

SEQ ID NO: 832 - DNA, 29 bp, Homo sapiens
  modified_base (15)..(15): a, c, g, t, unknown or other
ggttcaacagagtcnatgagaaaatcctt

SEQ ID NO: 833 - DNA, 29 bp, Homo sapiens
  modified_base (15)..(15): a, c, g, t, unknown or other
tacacagaaggccantgaatacatatctt

SEQ ID NO: 834 - DNA, 29 bp, Homo sapiens
  modified_base (15)..(15): a, c, g, t, unknown or other
atcatgtttccaaanagaaattagtgtat

SEQ ID NO: 835 - DNA, 29 bp, Homo sapiens
  modified_base (15)..(15): a, c, g, t, unknown or other
aggatgatgttgaanacattgaaatggag

SEQ ID NO: 836 - DNA, 29 bp, Homo sapiens
  modified_base (15)..(15): a, c, g, t, unknown or other
caatgtctagtatgncattttcaccaatt

SEQ ID NO: 837 - DNA, 29 bp, Homo sapiens
  modified_base (15)..(15): a, c, g, t, unknown or other
tttttaaagacagcntgtcactcatgaaa

SEQ ID NO: 838 - DNA, 29 bp, Homo sapiens
  modified_base (15)..(15): a, c, g, t, unknown or other
ttctccaaacatacncatttctagtttag

SEQ ID NO: 839 - DNA, 29 bp, Homo sapiens
  modified_base (15)..(15): a, c, g, t, unknown or other
aataagtgaaatacntcagatagttaaag

SEQ ID NO: 840 - DNA, 29 bp, Homo sapiens
  modified_base (15)..(15): a, c, g, t, unknown or other
actagtttattcttnagagcaatacaaat

SEQ ID NO: 841 - DNA, 29 bp, Homo sapiens
  modified_base (15)..(15): a, c, g, t, unknown or other
caatggaatccaaanatttgtttaatcta

SEQ ID NO: 842 - DNA, 29 bp, Homo sapiens
  modified_base (15)..(15): a, c, g, t, unknown or other
ggaagagatgctgantgaggtaatttgag

SEQ ID NO: 843 - DNA, 29 bp, Homo sapiens
  modified_base (15)..(15): a, c, g, t, unknown or other
aaaactatccttcgncatggtgtcaggta

SEQ ID NO: 844 - DNA, 29 bp, Homo sapiens
  modified_base (15)..(15): a, c, g, t, unknown or other
attttgagtagttgnaattttgattaata

SEQ ID NO: 845 - DNA, 29 bp, Homo sapiens
  modified_base (15)..(15): a, c, g, t, unknown or other
ttttcattataaatncacatgctgtcatc

SEQ ID NO: 846 - DNA, 29 bp, Homo sapiens
  modified_base (15)..(15): a, c, g, t, unknown or other
aatgtctgtacacanaggctaccaggcta

SEQ ID NO: 847 - DNA, 29 bp, Homo sapiens
  modified_base (15)..(15): a, c, g, t, unknown or other
agcagtaaactctangcacactgggctat

SEQ ID NO: 848 - DNA, 29 bp, Homo sapiens
  modified_base (15)..(15): a, c, g, t, unknown or other
atgggtagcatatancattgactcaggtc

SEQ ID NO: 849 - DNA, 29 bp, Homo sapiens
  modified_base (15)..(15): a, c, g, t, unknown or other
tgtgtttttgcctcnaagagggatttgcc

SEQ ID NO: 850 - DNA, 29 bp, Homo sapiens
  modified_base (15)..(15): a, c, g, t, unknown or other
gaaattcaagattcntatatgtaaagtcc

SEQ ID NO: 851 - DNA, 29 bp, Homo sapiens
  modified_base (15)..(15): a, c, g, t, unknown or other
ctcacaatgtcaaangtcaaattcagaac

SEQ ID NO: 852 - DNA, 29 bp, Homo sapiens
  modified_base (15)..(15): a, c, g, t, unknown or other
tttaaatggcagtanaagaaatactgatt

SEQ ID NO: 853 - DNA, 29 bp, Homo sapiens
  modified_base (15)..(15): a, c, g, t, unknown or other
taaaaataacatgcnctcattaactgaga

SEQ ID NO: 854 - DNA, 29 bp, Homo sapiens
  modified_base (15)..(15): a, c, g, t, unknown or other
ctccagacagtgtgnttctactagaaagc

SEQ ID NO: 855 - DNA, 29 bp, Homo sapiens
  modified_base (15)..(15): a, c, g, t, unknown or other
tggtggatctttganaacaagtgaccatg

SEQ ID NO: 856 - DNA, 29 bp, Homo sapiens
  modified_base (15)..(15): a, c, g, t, unknown or other
taattatcataaatnaagagtattgtatc

SEQ ID NO: 857 - DNA, 29 bp, Homo sapiens
  modified_base (15)..(15): a, c, g, t, unknown or other
tttacctgtctattncacaactgttattg

SEQ ID NO: 858 - DNA, 29 bp, Homo sapiens
  modified_base (15)..(15): a, c, g, t, unknown or other
tctggttggcatatnctcctccattccat

SEQ ID NO: 859 - DNA, 29 bp, Homo sapiens
  modified_base (15)..(15): a, c, g, t, unknown or other
gtctgcaaagaaacngttagggaatttta

SEQ ID NO: 860 - DNA, 29 bp, Homo sapiens
  modified_base (15)..(15): a, c, g, t, unknown or other
acataaacatgttgnggataagctccaca

SEQ ID NO: 861 - DNA, 29 bp, Homo sapiens
  modified_base (15)..(15): a, c, g, t, unknown or other
tttctccctatgaanctcttttcacccag

SEQ ID NO: 862 - DNA, 29 bp, Homo sapiens
  modified_base (15)..(15): a, c, g, t, unknown or other
cttccatcctcttanagtatttccctaca

SEQ ID NO: 863 - DNA, 29 bp, Homo sapiens
  modified_base (15)..(15): a, c, g, t, unknown or other
agggcaacattttanacacaatggggata

SEQ ID NO: 864 - DNA, 29 bp, Homo sapiens
  modified_base (15)..(15): a, c, g, t, unknown or other
ctggttcttcaatantacagtgattttac

SEQ ID NO: 865 - DNA, 29 bp, Homo sapiens
  modified_base (15)..(15): a, c, g, t, unknown or other
acaagtgagtgaganacattcagactttt

SEQ ID NO: 866 - DNA, 29 bp, Homo sapiens
  modified_base (15)..(15): a, c, g, t, unknown or other
gtgatattcactgtnttaaagaaaccgga

SEQ ID NO: 867 - DNA, 29 bp, Homo sapiens
  modified_base (15)..(15): a, c, g, t, unknown or other
caaaagctgtcattntgagacaagttctc

SEQ ID NO: 868 - DNA, 29 bp, Homo sapiens
  modified_base (15)..(15): a, c, g, t, unknown or other
atctaacctggtggncgtcctactgggac

SEQ ID NO: 869 - DNA, 29 bp, Homo sapiens
  modified_base (15)..(15): a, c, g, t, unknown or other
gattaatcatagccngtcaatacgaaagc

SEQ ID NO: 870 - DNA, 29 bp, Homo sapiens
  modified_base (15)..(15): a, c, g, t, unknown or other
ttttaaaattccaangtaaaggtctcaca

SEQ ID NO: 871 - DNA, 29 bp, Homo sapiens
  modified_base (15)..(15): a, c, g, t, unknown or other
atctatttgctcagnattctctattcttt

SEQ ID NO: 872 - DNA, 29 bp, Homo sapiens
  modified_base (15)..(15): a, c, g, t, unknown or other
tctacatgcgctcangaaaacaatgcctg

SEQ ID NO: 873 - DNA, 29 bp, Homo sapiens
  modified_base (15)..(15): a, c, g, t, unknown or other
tatatttttactccnaatactgtttcaca

SEQ ID NO: 874 - DNA, 29 bp, Homo sapiens
  modified_base (15)..(15): a, c, g, t, unknown or other
atgtttcaaaactgnttaggacatcaaga

SEQ ID NO: 875 - DNA, 29 bp, Homo sapiens
  modified_base (15)..(15): a, c, g, t, unknown or other
gggctgtgctgagcntcctccaccttcat

SEQ ID NO: 876 - DNA, 29 bp, Homo sapiens
  modified_base (15)..(15): a, c, g, t, unknown or other
ttggaagcatgttgncacctacagtagta

SEQ ID NO: 877 - DNA, 29 bp, Homo sapiens
  modified_base (15)..(15): a, c, g, t, unknown or other
cagcctgttgaattngcaatgtcaatgtc

SEQ ID NO: 878 - DNA, 29 bp, Homo sapiens
  modified_base (15)..(15): a, c, g, t, unknown or other
aggaagcctaagatngatttcacctggct

SEQ ID NO: 879 - DNA, 29 bp, Homo sapiens
  modified_base (15)..(15): a, c, g, t, unknown or other
atttagagggaacgnaaattaaccaaatc

SEQ ID NO: 880 - DNA, 29 bp, Homo sapiens
  modified_base (15)..(15): a, c, g, t, unknown or other
aacaggtcatgatgnttcagaaaagcaaa

SEQ ID NO: 881 - DNA, 29 bp, Homo sapiens
  modified_base (15)..(15): a, c, g, t, unknown or other
gaaggatcctagtancatttcagacattt

SEQ ID NO: 882 - DNA, 29 bp, Homo sapiens
  modified_base (15)..(15): a, c, g, t, unknown or other
ttcaggggtgtgatncagtgcattgatgg

SEQ ID NO: 883 - DNA, 29 bp, Homo sapiens
  modified_base (15)..(15): a, c, g, t, unknown or other
ggacatctggccagntggagagatccacc

SEQ ID NO: 884 - DNA, 29 bp, Homo sapiens
  modified_base (15)..(15): a, c, g, t, unknown or other
cgtgatctgtgaacnatatcaagggtcat

SEQ ID NO: 885 - DNA, 29 bp, Homo sapiens
  modified_base (15)..(15): a, c, g, t, unknown or other
atatattttgttttnaagaaaattgagag

SEQ ID NO: 886 - DNA, 29 bp, Homo sapiens
  modified_base (15)..(15): a, c, g, t, unknown or other
agattgtggtatatncataaagcgaaata

SEQ ID NO: 887 - DNA, 29 bp, Homo sapiens
  modified_base (15)..(15): a, c, g, t, unknown or other
ttaggatgtaagagngtcttttactacag

SEQ ID NO: 888 - DNA, 29 bp, Homo sapiens
  modified_base (15)..(15): a, c, g, t, unknown or other
aaacagaatgatgantactaagctattca

SEQ ID NO: 889 - DNA, 29 bp, Homo sapiens
  modified_base (15)..(15): a, c, g, t, unknown or other
gaacagtttttttanaattggaatgtagt

SEQ ID NO: 890 - DNA, 29 bp, Homo sapiens
  modified_base (15)..(15): a, c, g, t, unknown or other
ttatgctcctttctnttccaatcccagga

SEQ ID NO: 891 - DNA, 29 bp, Homo sapiens
  modified_base (15)..(15): a, c, g, t, unknown or other
gagaccagtcgttgngttgatgacactgt

SEQ ID NO: 892 - DNA, 29 bp, Homo sapiens
  modified_base (15)..(15): a, c, g, t, unknown or other
tgcgctgaatggtanatatggtaagaaat

SEQ ID NO: 893 - DNA, 29 bp, Homo sapiens
  modified_base (15)..(15): a, c, g, t, unknown or other
gctgggatttgcttnacagagaaggaggc

SEQ ID NO: 894 - DNA, 29 bp, Homo sapiens
  modified_base (15)..(15): a, c, g, t, unknown or other
ttttatattgacccntggctactgtggta

SEQ ID NO: 895 - DNA, 29 bp, Homo sapiens
  modified_base (15)..(15): a, c, g, t, unknown or other
taagggcacagtttntatcttctacctct

SEQ ID NO: 896 - DNA, 29 bp, Homo sapiens
  modified_base (15)..(15): a, c, g, t, unknown or other
ggatggagagggagncagtgatgttctga

SEQ ID NO: 897 - DNA, 29 bp, Homo sapiens
  modified_base (15)..(15): a, c, g, t, unknown or other
gtgtctgtggcctgnctgagctccggaag

SEQ ID NO: 898 - DNA, 29 bp, Homo sapiens
  modified_base (15)..(15): a, c, g, t, unknown or other
ctgtatgacggaccngtgtgtgaagtgtc

SEQ ID NO: 899 - DNA, 29 bp, Homo sapiens
  modified_base (15)..(15): a, c, g, t, unknown or other
cagcctgcgggtgcntggcaaaccaagtg

SEQ ID NO: 900 - DNA, 29 bp, Homo sapiens
  modified_base (15)..(15): a, c, g, t, unknown or other
tcctctcccctgtgnatcttttctcaaag

SEQ ID NO: 901 - DNA, 29 bp, Homo sapiens
  modified_base (15)..(15): a, c, g, t, unknown or other
aagagagtttagatncctaaaagaattga

SEQ ID NO: 902 - DNA, 29 bp, Homo sapiens
  modified_base (15)..(15): a, c, g, t, unknown or other
ataagcaaaaggctncaaaacaatggcag

SEQ ID NO: 903 - DNA, 29 bp, Homo sapiens
  modified_base (15)..(15): a, c, g, t, unknown or other
gaagtatagttttcnatgctctgatcttg

SEQ ID NO: 904 - DNA, 29 bp, Homo sapiens
  modified_base (15)..(15): a, c, g, t, unknown or other
ttgttaagaggcacntgagcatagtgctg

SEQ ID NO: 905 - DNA, 29 bp, Homo sapiens
  modified_base (15)..(15): a, c, g, t, unknown or other
gatgcagccttcctntacatttccattct

SEQ ID NO: 906 - DNA, 29 bp, Homo sapiens
  modified_base (15)..(15): a, c, g, t, unknown or other
tttcaccgccattgntcattcacttttcc

SEQ ID NO: 907 - DNA, 29 bp, Homo sapiens
  modified_base (15)..(15): a, c, g, t, unknown or other
ctggctgaatacagnttaattggtgtcaa

SEQ ID NO: 908 - DNA, 29 bp, Homo sapiens
  modified_base (15)..(15): a, c, g, t, unknown or other
gggaaggtcaagggnttttgtcaatgcag

SEQ ID NO: 909 - DNA, 29 bp, Homo sapiens
  modified_base (15)..(15): a, c, g, t, unknown or other
atcatccaggcatcngcgtgctcttgcaa

SEQ ID NO: 910 - DNA, 29 bp, Homo sapiens
  modified_base (15)..(15): a, c, g, t, unknown or other
ggcacaacctcagantgattatatttttg

SEQ ID NO: 911 - DNA, 29 bp, Homo sapiens
  modified_base (15)..(15): a, c, g, t, unknown or other
cctgatggttttcantgagaatgagatac

SEQ ID NO: 912 - DNA, 29 bp, Homo sapiens
  modified_base (15)..(15): a, c, g, t, unknown or other
gacaagtttagtcanagctttattcctaa

SEQ ID NO: 913 - DNA, 29 bp, Homo sapiens
  modified_base (15)..(15): a, c, g, t, unknown or other
cccagaaaataccanactgatttcctcat

SEQ ID NO: 914 - DNA, 29 bp, Homo sapiens
  modified_base (15)..(15): a, c, g, t, unknown or other
atgactgtcaagaancaagaacttgttga

SEQ ID NO: 915 - DNA, 29 bp, Homo sapiens
  modified_base (15)..(15): a, c, g, t, unknown or other
atgaagctgaaaaanaatttggaagtcaa

SEQ ID NO: 916 - DNA, 29 bp, Homo sapiens
  modified_base (15)..(15): a, c, g, t, unknown or other
tctcaaatgtgtacngtacaggatttgct

SEQ ID NO: 917 - DNA, 29 bp, Homo sapiens
  modified_base (15)..(15): a, c, g, t, unknown or other
tatggaaacaatcanatgatgtagcagta

SEQ ID NO: 918 - DNA, 29 bp, Homo sapiens
  modified_base (15)..(15): a, c, g, t, unknown or other
tagaaaaaaatgaanccgtctcttttcag

SEQ ID NO: 919 - DNA, 29 bp, Homo sapiens
  modified_base (15)..(15): a, c, g, t, unknown or other
gcttacaaatgtgantcataaatgacttg

SEQ ID NO: 920 - DNA, 29 bp, Homo sapiens
  modified_base (15)..(15): a, c, g, t, unknown or other
actaaaattggaaangttgtaagtcatct

SEQ ID NO: 921 - DNA, 29 bp, Homo sapiens
  modified_base (15)..(15): a, c, g, t, unknown or other
ttcctgagtgatacnctgggtaaaggtga

SEQ ID NO: 922 - DNA, 29 bp, Homo sapiens
  modified_base (15)..(15): a, c, g, t, unknown or other
agcgtgaggattacnagaggcatatgtgg

SEQ ID NO: 923 - DNA, 29 bp, Homo sapiens
  modified_base (15)..(15): a, c, g, t, unknown or other
tgctgctctcagttncaaaagggatccag

SEQ ID NO: 924 - DNA, 29 bp, Homo sapiens
  modified_base (15)..(15): a, c, g, t, unknown or other
atattagatagaagnctttgctacatgta

SEQ ID NO: 925 - DNA, 29 bp, Homo sapiens
  modified_base (15)..(15): a, c, g, t, unknown or other
ttcagttgagcttcntgactcgtttctag

SEQ ID NO: 926 - DNA, 29 bp, Homo sapiens
  modified_base (15)..(15): a, c, g, t, unknown or other
acagacaatgtctantggctgtgctgtac

SEQ ID NO: 927 - DNA, 29 bp, Homo sapiens
  modified_base (15)..(15): a, c, g, t, unknown or other
tttcatctatgtagnaatatgtgatagca

SEQ ID NO: 928 - DNA, 29 bp, Homo sapiens
  modified_base (15)..(15): a, c, g, t, unknown or other
tacactttagtggangagacaggaaaaca

SEQ ID NO: 929 - DNA, 29 bp, Homo sapiens
  modified_base (15)..(15): a, c, g, t, unknown or other
gtagagtgcagcaanctgcttatagaaaa

SEQ ID NO: 930 - DNA, 29 bp, Homo sapiens
  modified_base (15)..(15): a, c, g, t, unknown or other
cctaatggaatgtanacttattaaccttt

SEQ ID NO: 931 - DNA, 29 bp, Homo sapiens
  modified_base (15)..(15): a, c, g, t, unknown or other
acaaacatgtgttanagatacaccatgtg

SEQ ID NO: 932 - DNA, 29 bp, Homo sapiens
  modified_base (15)..(15): a, c, g, t, unknown or other
agccttagagtctcncttaatcctctgat

SEQ ID NO: 933 - DNA, 29 bp, Homo sapiens
  modified_base (15)..(15): a, c, g, t, unknown or other
atgtacatcacctangaagtattcttgtt

SEQ ID NO: 934 - DNA, 29 bp, Homo sapiens
  modified_base (15)..(15): a, c, g, t, unknown or other
ctccaagaagtcacnattaagcttataag

SEQ ID NO: 935 - DNA, 29 bp, Homo sapiens
  modified_base (15)..(15): a, c, g, t, unknown or other
gtcatctccctgganctcagactcatgca

SEQ ID NO: 936 - DNA, 29 bp, Homo sapiens
  modified_base (15)..(15): a, c, g, t, unknown or other
accagagtgagaggntgaggtaaggcaga

SEQ ID NO: 937 - DNA, 29 bp, Homo sapiens
  modified_base (15)..(15): a, c, g, t, unknown or other
tttctttttttaccnttattgttgaaaac

SEQ ID NO: 938 - DNA, 29 bp, Homo sapiens
  modified_base (15)..(15): a, c, g, t, unknown or other
cagtgaaaaggcaanggatttacgtactt

SEQ ID NO: 939 - DNA, 29 bp, Homo sapiens
  modified_base (15)..(15): a, c, g, t, unknown or other
atgatgatatctgancaaagatctaaata

SEQ ID NO: 940 - DNA, 29 bp, Homo sapiens
  modified_base (15)..(15): a, c, g, t, unknown or other
agaacaaagaaagangtgaactgacactc

SEQ ID NO: 941 - DNA, 29 bp, Homo sapiens
  modified_base (15)..(15): a, c, g, t, unknown or other
cacatcatgctgcangccagggaactata

SEQ ID NO: 942 - DNA, 29 bp, Homo sapiens
  modified_base (15)..(15): a, c, g, t, unknown or other
cacctggcggcatcnagggagcaaggagg

SEQ ID NO: 943 - DNA, 29 bp, Homo sapiens
  modified_base (15)..(15): a, c, g, t, unknown or other
ccccacaaacagggnctaacacaagctcg

SEQ ID NO: 944 - DNA, 29 bp, Homo sapiens
  modified_base (15)..(15): a, c, g, t, unknown or other
gtggtttaagacacntatcatattcacgg

SEQ ID NO: 945 - DNA, 29 bp, Homo sapiens
  modified_base (15)..(15): a, c, g, t, unknown or other
ctctgagtgccctgnctctatctctggtc

SEQ ID NO: 946 - DNA, 29 bp, Homo sapiens
  modified_base (15)..(15): a, c, g, t, unknown or other
gtccttccaatctcnatggaggcacccaa

SEQ ID NO: 947 - DNA, 29 bp, Homo sapiens
  modified_base (15)..(15): a, c, g, t, unknown or other
caactgcaacaatgncaagtgctcctaga

SEQ ID NO: 948 - DNA, 29 bp, Homo sapiens
  modified_base (15)..(15): a, c, g, t, unknown or other
tcagtatagagcatngaatcacaactcct

SEQ ID NO: 949 - DNA, 29 bp, Homo sapiens
  modified_base (15)..(15): a, c, g, t, unknown or other
tcctccatgtaaaantcctccacaaaaag

SEQ ID NO: 950 - DNA, 29 bp, Homo sapiens
  modified_base (15)..(15): a, c, g, t, unknown or other
tccagccaggaagangatcacgcatgcca

SEQ ID NO: 951 - DNA, 29 bp, Homo sapiens
  modified_base (15)..(15): a, c, g, t, unknown or other
tgtcacctttcctcnagttttttccacca

SEQ ID NO: 952 - DNA, 29 bp, Homo sapiens
  modified_base (15)..(15): a, c, g, t, unknown or other
gtcttctttgacctntcctgctccctgca

SEQ ID NO: 953 - DNA, 29 bp, Homo sapiens
  modified_base (15)..(15): a, c, g, t, unknown or other
ctttcaaaagctgtngaatccttctttga

SEQ ID NO: 954 - DNA, 29 bp, Homo sapiens
  modified_base (15)..(15): a, c, g, t, unknown or other
gtcagcgacacgttntgtgggaccctgag

SEQ ID NO: 955 - DNA, 29 bp, Homo sapiens
  modified_base (15)..(15): a, c, g, t, unknown or other
caatcatttcatcantagtccctgggcct

SEQ ID NO: 956 - DNA, 29 bp, Homo sapiens
  modified_base (15)..(15): a, c, g, t, unknown or other
gaggtgtagacatgnttcccgcctatcca

SEQ ID NO: 957 - DNA, 29 bp, Homo sapiens
  modified_base (15)..(15): a, c, g, t, unknown or other
cttattctctcttangcttctgtagtcaa

SEQ ID NO: 958 - DNA, 29 bp, Homo sapiens
  modified_base (15)..(15): a, c, g, t, unknown or other
aaaaatcagttgagnccacctatcctata

SEQ ID NO: 959 - DNA, 29 bp, Homo sapiens
  modified_base (15)..(15): a, c, g, t, unknown or other
ggaagccaagaatancgttggtgttgttg

SEQ ID NO: 960 - DNA, 29 bp, Homo sapiens
  modified_base (15)..(15): a, c, g, t, unknown or other
ggaaatgactaacanctgcttaagtcctt

SEQ ID NO: 961 - DNA, 29 bp, Homo sapiens
  modified_base (15)..(15): a, c, g, t, unknown or other
taacccatcttagcnagactccaggtttc

SEQ ID NO: 962 - DNA, 29 bp, Homo sapiens
  modified_base (15)..(15): a, c, g, t, unknown or other
cactcatcactatcngcatgttcgtggtc

SEQ ID NO: 963 - DNA, 29 bp, Homo sapiens
  modified_base (15)..(15): a, c, g, t, unknown or other
taaacagtagctccntccttaacatcagt

SEQ ID NO: 964 - DNA, 29 bp, Homo sapiens
  modified_base (15)..(15): a, c, g, t, unknown or other
cagcaactcagagcntatggaagactgtc

SEQ ID NO: 965 - DNA, 29 bp, Homo sapiens
  modified_base (15)..(15): a, c, g, t, unknown or other
gttaatactcaatcntctgtgaaattcaa

SEQ ID NO: 966 - DNA, 29 bp, Homo sapiens
  modified_base (15)..(15): a, c, g, t, unknown or other
tccgtgttgggatgngtggaaggttgcat

SEQ ID NO: 967 - DNA, 29 bp, Homo sapiens
  modified_base (15)..(15): a, c, g, t, unknown or other
ttatgaattacgttntgtccagttatgat

SEQ ID NO: 968 - DNA, 29 bp, Homo sapiens
  modified_base (15)..(15): a, c, g, t, unknown or other
aatagtaactcctcngagacttccaggga

SEQ ID NO: 969 - DNA, 29 bp, Homo sapiens
  modified_base (15)..(15): a, c, g, t, unknown or other
gtcaaaactgaactnttaaaattcttaca

SEQ ID NO: 970 - DNA, 29 bp, Homo sapiens
  modified_base (15)..(15): a, c, g, t, unknown or other
gcatttctaatcctntgtctcatcattta

SEQ ID NO: 971 - DNA, 29 bp, Homo sapiens
  modified_base (15)..(15): a, c, g, t, unknown or other
gacagtgggaatgcntagtgaatggctct

SEQ ID NO: 972 - DNA, 29 bp, Homo sapiens
  modified_base (15)..(15): a, c, g, t, unknown or other
gcataaagagatctngcccgatgtcaacc

SEQ ID NO: 973 - DNA, 29 bp, Homo sapiens
  modified_base (15)..(15): a, c, g, t, unknown or other
tcattctcatagtcngagtgtgtttccac

SEQ ID NO: 974 - DNA, 29 bp, Homo sapiens
  modified_base (15)..(15): a, c, g, t, unknown or other
ggctgtttctccggnatccacgtgccttt

SEQ ID NO: 975 - DNA, 29 bp, Homo sapiens
  modified_base (15)..(15): a, c, g, t, unknown or other
ttccaggtcatttcnagtcctgccttcga

SEQ ID NO: 976 - DNA, 29 bp, Homo sapiens
  modified_base (15)..(15): a, c, g, t, unknown or other
aagaaaacctttttnacaatgaccgcggg

SEQ ID NO: 977 - DNA, 29 bp, Homo sapiens
  modified_base (15)..(15): a, c, g, t, unknown or other
ccagaggtttgttcntaagtcaataggtg

SEQ ID NO: 978 - DNA, 29 bp, Homo sapiens
  modified_base (15)..(15): a, c, g, t, unknown or other
acctctctcccagcntagcaggataagta

SEQ ID NO: 979 - DNA, 29 bp, Homo sapiens
  modified_base (15)..(15): a, c, g, t, unknown or other
taacttattgttacnccttctgacaattt

SEQ ID NO: 980 - DNA, 29 bp, Homo sapiens
  modified_base (15)..(15): a, c, g, t, unknown or other
ctttgtttaggccangaatcttgcttttg

SEQ ID NO: 981 - DNA, 29 bp, Homo sapiens
  modified_base (15)..(15): a, c, g, t, unknown or other
ctttaacacactctnctcccttgaactct

SEQ ID NO: 982 - DNA, 29 bp, Homo sapiens
  modified_base (15)..(15): a, c, g, t, unknown or other
gtgacttccatactncaaatggtacatac

SEQ ID NO: 983 - DNA, 29 bp, Homo sapiens
  modified_base (15)..(15): a, c, g, t, unknown or other
gtgtcatcctggtanagagtaattccaga

SEQ ID NO: 984 - DNA, 29 bp, Homo sapiens
  modified_base (15)..(15): a, c, g, t, unknown or other
gaatggactgtctcntctatgatgcaaat

SEQ ID NO: 985 - DNA, 29 bp, Homo sapiens
  modified_base (15)..(15): a, c, g, t, unknown or other
tccactgttcatcanaatgtttcggtatg

SEQ ID NO: 986 - DNA, 29 bp, Homo sapiens
  modified_base (15)..(15): a, c, g, t, unknown or other
atccgaggagagtcntgcaaacaacccaa

SEQ ID NO: 987 - DNA, 29 bp, Homo sapiens
  modified_base (15)..(15): a, c, g, t, unknown or other
aagaagcccttcctngggagaatgccaga

SEQ ID NO: 988 - DNA, 29 bp, Homo sapiens
  modified_base (15)..(15): a, c, g, t, unknown or other
gtccaacacttcaantattttcctttaga

SEQ ID NO: 989 - DNA, 29 bp, Homo sapiens
  modified_base (15)..(15): a, c, g, t, unknown or other
cccatgcaaagtacntgtgaatatttgta

SEQ ID NO: 990 - DNA, 29 bp, Homo sapiens
  modified_base (15)..(15): a, c, g, t, unknown or other
taacctggtcctatntatgctctggatca

SEQ ID NO: 991 - DNA, 29 bp, Homo sapiens
  modified_base (15)..(15): a, c, g, t, unknown or other
atctagagatggatncagcaatcccaagg

SEQ ID NO: 992 - DNA, 29 bp, Homo sapiens
  modified_base (15)..(15): a, c, g, t, unknown or other
gaagtactcttatcngtctcgtgtgtgtg

SEQ ID NO: 993 - DNA, 29 bp, Homo sapiens
  modified_base (15)..(15): a, c, g, t, unknown or other
agaagcactttctcncaaaaggacaatgg

SEQ ID NO: 994 - DNA, 29 bp, Homo sapiens
  modified_base (15)..(15): a, c, g, t, unknown or other
caaattctagctcangttgccttccaagc

SEQ ID NO: 995 - DNA, 29 bp, Homo sapiens
  modified_base (15)..(15): a, c, g, t, unknown or other
ttcgccaatcttatnacccaaactcagat

SEQ ID NO: 996 - DNA, 29 bp, Homo sapiens
  modified_base (15)..(15): a, c, g, t, unknown or other
gcaggctgatttganggtttttgtcagcg

SEQ ID NO: 997 - DNA, 29 bp, Homo sapiens
  modified_base (15)..(15): a, c, g, t, unknown or other
ctacttctacctacnatgcattgagcact

SEQ ID NO: 998 - DNA, 29 bp, Homo sapiens
  modified_base (15)..(15): a, c, g, t, unknown or other
tatgtcttgagtctnagcaatgacattca

SEQ ID NO: 999 - DNA, 29 bp, Homo sapiens
  modified_base (15)..(15): a, c, g, t, unknown or other
ctgttcaatttgtgncacttgaagagtca

SEQ ID NO: 1000 - DNA, 29 bp, Homo sapiens
  modified_base (15)..(15): a, c, g, t, unknown or other
ctgtcataaccaganatgcaggaaggaga

SEQ ID NO: 1001 - DNA, 29 bp, Homo sapiens
  modified_base (15)..(15): a, c, g, t, unknown or other
taagtgcctgctccntaaagtgaaatctc

SEQ ID NO: 1002 - DNA, 29 bp, Homo sapiens
  modified_base (15)..(15): a, c, g, t, unknown or other
ttctgagaaagtganagtgtaataagata

SEQ ID NO: 1003 - DNA, 29 bp, Homo sapiens
  modified_base (15)..(15): a, c, g, t, unknown or other
tccctccatggctgnggctcagacacaca

SEQ ID NO: 1004 - DNA, 29 bp, Homo sapiens
  modified_base (15)..(15): a, c, g, t, unknown or other
ttctttttagtttcntgggccaaggggtg

SEQ ID NO: 1005 - DNA, 29 bp, Homo sapiens
  modified_base (15)..(15): a, c, g, t, unknown or other
caattcaagattacntcactataatttac

SEQ ID NO: 1006 - DNA, 29 bp, Homo sapiens
  modified_base (15)..(15): a, c, g, t, unknown or other
tttgggtttttccantggatccttcacaa

SEQ ID NO: 1007 - DNA, 29 bp, Homo sapiens
  modified_base (15)..(15): a, c, g, t, unknown or other
cggaaggtcctgctncaagcctgagaatt

SEQ ID NO: 1008 - DNA, 29 bp, Homo sapiens
  modified_base (15)..(15): a, c, g, t, unknown or other
ctctgtatctgagcnagggtgtgcggaag

SEQ ID NO: 1009 - DNA, 29 bp, Homo sapiens
  modified_base (15)..(15): a, c, g, t, unknown or other
agctctgtaagcagngtggcccccatagc

SEQ ID NO: 1010 - DNA, 29 bp, Homo sapiens
  modified_base (15)..(15): a, c, g, t, unknown or other
ctgcattagagttanaaaacaaagtcata

SEQ ID NO: 1011 - DNA, 29 bp, Homo sapiens
  modified_base (15)..(15): a, c, g, t, unknown or other
cccagtgtcagtganggcattaggaggca

SEQ ID NO: 1012 - DNA, 29 bp, Homo sapiens
  modified_base (15)..(15): a, c, g, t, unknown or other
ttcaagatcccactntccatccgccagaa

SEQ ID NO: 1013 - DNA, 29 bp, Homo sapiens
  modified_base (15)..(15): a, c, g, t, unknown or other
ttcccttgcttgctnttactaagacggtc

SEQ ID NO: 1014 - DNA, 29 bp, Homo sapiens
  modified_base (15)..(15): a, c, g, t, unknown or other
acaccactggagatngtcaggacagggac

SEQ ID NO: 1015 - DNA, 29 bp, Homo sapiens
  modified_base (15)..(15): a, c, g, t, unknown or other
agggagaggaatganagctgatgcaccaa

SEQ ID NO: 1016 - DNA, 29 bp, Homo sapiens
  modified_base (15)..(15): a, c, g, t, unknown or other
accctggtggctctntaagcaacttgagt

SEQ ID NO: 1017 - DNA, 29 bp, Homo sapiens
  modified_base (15)..(15): a, c, g, t, unknown or other
aatgtaggtaacttnataaactgcctcag

SEQ ID NO: 1018 - DNA, 29 bp, Homo sapiens
  modified_base (15)..(15): a, c, g, t, unknown or other
tgtatttttctcctnttgctgcaagtctc

SEQ ID NO: 1019 - DNA, 29 bp, Homo sapiens
  modified_base (15)..(15): a, c, g, t, unknown or other
gtcattttcgtaagnagacagactctagg

SEQ ID NO: 1020 - DNA, 29 bp, Homo sapiens
  modified_base (15)..(15): a, c, g, t, unknown or other
ttctcttaaaaatancagcatgtctgtgg

SEQ ID NO: 1021 - DNA, 29 bp, Homo sapiens
  modified_base (15)..(15): a, c, g, t, unknown or other
atggattcttaaagngacagaagatgctc

SEQ ID NO: 1022 - DNA, 29 bp, Homo sapiens
  modified_base (15)..(15): a, c, g, t, unknown or other
ctttctacaagtacngctaacctgatctt

SEQ ID NO: 1023 - DNA, 29 bp, Homo sapiens
  modified_base (15)..(15): a, c, g, t, unknown or other
tgcgctccattatcncaggcatttgtagc

SEQ ID NO: 1024 - DNA, 29 bp, Homo sapiens
  modified_base (15)..(15): a, c, g, t, unknown or other
tatggcctggtaccngatctcaggagaag

SEQ ID NO: 1025 - DNA, 29 bp, Homo sapiens
  modified_base (15)..(15): a, c, g, t, unknown or other
ctacccaaagtttgntaagaaaatgtgct

SEQ ID NO: 1026 - DNA, 29 bp, Homo sapiens
  modified_base (15)..(15): a, c, g, t, unknown or other
cccagctctatgacngacatcatgtggat

SEQ ID NO: 1027 - DNA, 29 bp, Homo sapiens
  modified_base (15)..(15): a, c, g, t, unknown or other
tgtaatgagagcagngaacataaatcacc

SEQ ID NO: 1028 - DNA, 29 bp, Homo sapiens
  modified_base (15)..(15): a, c, g, t, unknown or other
ctgaagtcttcctantaagatcaaaagta

SEQ ID NO: 1029 - DNA, 29 bp, Homo sapiens
  modified_base (15)..(15): a, c, g, t, unknown or other
cctccacttcttggngcagcaggacttgg

SEQ ID NO: 1030 - DNA, 29 bp, Homo sapiens
  modified_base (15)..(15): a, c, g, t, unknown or other
gccccattccttccntgttccttaaatat

SEQ ID NO: 1031 - DNA, 29 bp, Homo sapiens
  modified_base (15)..(15): a, c, g, t, unknown or other
tggtgataaataaanaatggcaacacact

SEQ ID NO: 1032 - DNA, 29 bp, Homo sapiens
  modified_base (15)..(15): a, c, g, t, unknown or other
tagaaaagccactantgatgagaggtagg

SEQ ID NO: 1033 - DNA, 29 bp, Homo sapiens
  modified_base (15)..(15): a, c, g, t, unknown or other
tattcagaaataacnagaaatggccctca

SEQ ID NO: 1034 - DNA, 29 bp, Homo sapiens
  modified_base (15)..(15): a, c, g, t, unknown or other
cgatcaactgatgcnccactggtctccaa

SEQ ID NO: 1035 - DNA, 29 bp, Homo sapiens
  modified_base (15)..(15): a, c, g, t, unknown or other
ctctgcttaatgtanaggtcatttttcaa

SEQ ID NO: 1036 - DNA, 29 bp, Homo sapiens
  modified_base (15)..(15): a, c, g, t, unknown or other
gcttcaggcctccantcaaacgccctctt

SEQ ID NO: 1037 - DNA, 29 bp, Homo sapiens
  modified_base (15)..(15): a, c, g, t, unknown or other
tcatctttccccccnacaactggagacac

SEQ ID NO: 1038 - DNA, 29 bp, Homo sapiens
  modified_base (15)..(15): a, c, g, t, unknown or other
gtgttgggacagctncgtttaaaaaggca

SEQ ID NO: 1039 - DNA, 29 bp, Homo sapiens
  modified_base (15)..(15): a, c, g, t, unknown or other
ttaaaaaacaagggncctgtaaaaccctt

SEQ ID NO: 1040 - DNA, 29 bp, Homo sapiens
  modified_base (15)..(15): a, c, g, t, unknown or other
tgagtggcctttagnggagctcaagcttt

SEQ ID NO: 1041 - DNA, 29 bp, Homo sapiens
  modified_base (15)..(15): a, c, g, t, unknown or other
atctgggctagtttncccctccttaggca

SEQ ID NO: 1042 - DNA, 29 bp, Homo sapiens
  modified_base (15)..(15): a, c, g, t, unknown or other
tttttaaaatgaganagaccttcaaaggg

SEQ ID NO: 1043 - DNA, 29 bp, Homo sapiens
  modified_base (15)..(15): a, c, g, t, unknown or other
gggtgcatttgtgcnggggttaacaatct

SEQ ID NO: 1044 - DNA, 29 bp, Homo sapiens
  modified_base (15)..(15): a, c, g, t, unknown or other
tgcagtttctttgtngcagatcccacaag

SEQ ID NO: 1045 - DNA, 29 bp, Homo sapiens
  modified_base (15)..(15): a, c, g, t, unknown or other
ctcagatgaaggtgncaccaattcttcaa

SEQ ID NO: 1046 - DNA, 29 bp, Homo sapiens
  modified_base (15)..(15): a, c, g, t, unknown or other
gtccaggaaactagnagcagaatggctgt

SEQ ID NO: 1047 - DNA, 29 bp, Homo sapiens
  modified_base (15)..(15): a, c, g, t, unknown or other
tacttaaaagataanatatctgtgacttg

SEQ ID NO: 1048 - DNA, 29 bp, Homo sapiens
  modified_base (15)..(15): a, c, g, t, unknown or other
tttaccatccatagngcctcaaatatgtc

SEQ ID NO: 1049 - DNA, 29 bp, Homo sapiens
  modified_base (15)..(15): a, c, g, t, unknown or other
aattgaaagttctanaaacagatgaagat

SEQ ID NO: 1050 - DNA, 29 bp, Homo sapiens
  modified_base (15)..(15): a, c, g, t, unknown or other
ttcttttactgcatntcaaaggtggaaag

SEQ ID NO: 1051 - DNA, 29 bp, Homo sapiens
  modified_base (15)..(15): a, c, g, t, unknown or other
tctcagagctaactngcagaaagcctggc

SEQ ID NO: 1052 - DNA, 29 bp, Homo sapiens
  modified_base (15)..(15): a, c, g, t, unknown or other
aaaacatagggtggnaaatacactaaaaa

SEQ ID NO: 1053 - DNA, 29 bp, Homo sapiens
  modified_base (15)..(15): a, c, g, t, unknown or other
attattttagacccnagtcccacacgaat

SEQ ID NO: 1054 - DNA, 29 bp, Homo sapiens
  modified_base (15)..(15): a, c, g, t, unknown or other
gccatcttttcaagnacaataggagcatc

SEQ ID NO: 1055 - DNA, 29 bp, Homo sapiens
  modified_base (15)..(15): a, c, g, t, unknown or other
ctgcctttgggtagnggtactatgtaaga

SEQ ID NO: 1056 - DNA, 29 bp, Homo sapiens
  modified_base (15)..(15): a, c, g, t, unknown or other
cccagcactttccantgtactgactagtc

SEQ ID NO: 1057 - DNA, 29 bp, Homo sapiens
  modified_base (15)..(15): a, c, g, t, unknown or other
ggcacaccattgtgncaaaggttatactg

SEQ ID NO: 1058 - DNA, 29 bp, Homo sapiens
  modified_base (15)..(15): a, c, g, t, unknown or other
tcctctttcttgacncctagggctgtgaa

SEQ ID NO: 1059 - DNA, 29 bp, Homo sapiens
  modified_base (15)..(15): a, c, g, t, unknown or other
atttaaagttccctntaaacaaatcacta

SEQ ID NO: 1060 - DNA, 29 bp, Homo sapiens
  modified_base (15)..(15): a, c, g, t, unknown or other
atttctacagcatgnttctttcggacaga

SEQ ID NO: 1061 - DNA, 29 bp, Homo sapiens
  modified_base (15)..(15): a, c, g, t, unknown or other
aagaggttctctcanttacgtgataccct

SEQ ID NO: 1062 - DNA, 29 bp, Homo sapiens
  modified_base (15)..(15): a, c, g, t, unknown or other
tctgggccttgaagncaaactaagggaat

SEQ ID NO: 1063 - DNA, 29 bp, Homo sapiens
  modified_base (15)..(15): a, c, g, t, unknown or other
attacacctgtacangactcttagccatg

SEQ ID NO: 1064 - DNA, 29 bp, Homo sapiens
  modified_base (15)..(15): a, c, g, t, unknown or other
gaggaagtcaaaagngccacctgtgaaaa

SEQ ID NO: 1065 - DNA, 29 bp, Homo sapiens
  modified_base (15)..(15): a, c, g, t, unknown or other
ggtggcatagctgantgatgtataaaatg

SEQ ID NO: 1066 - DNA, 29 bp, Homo sapiens
  modified_base (15)..(15): a, c, g, t, unknown or other
agttcaaaaacacangtagcagcagcagt

SEQ ID NO: 1067 - DNA, 29 bp, Homo sapiens
  modified_base (15)..(15): a, c, g, t, unknown or other
ttcattagtaggaanctcaaacttactct

SEQ ID NO: 1068 - DNA, 29 bp, Homo sapiens
  modified_base (15)..(15): a, c, g, t, unknown or other
gcaaacaacactttntacagtgaaactca

SEQ ID NO: 1069 - DNA, 29 bp, Homo sapiens
  modified_base (15)..(15): a, c, g, t, unknown or other
catgatggggaaacngtttttcactaacc

SEQ ID NO: 1070 - DNA, 29 bp, Homo sapiens
  modified_base (15)..(15): a, c, g, t, unknown or other
ttgaattactaatangcaacctcaccatt

SEQ ID NO: 1071 - DNA, 29 bp, Homo sapiens
  modified_base (15)..(15): a, c, g, t, unknown or other
agtcaggacgtctcncctaatgatattat

SEQ ID NO: 1072 - DNA, 29 bp, Homo sapiens
  modified_base (15)..(15): a, c, g, t, unknown or other
tatcaatttcttacntgtttagcaagtac

SEQ ID NO: 1073 - DNA, 29 bp, Homo sapiens
  modified_base (15)..(15): a, c, g, t, unknown or other
gaactgccagctccngattcattttcctt

SEQ ID NO: 1074 - DNA, 29 bp, Homo sapiens
  modified_base (15)..(15): a, c, g, t, unknown or other
gggaccttatctatngattacctatacca

SEQ ID NO: 1075 - DNA, 29 bp, Homo sapiens
  modified_base (15)..(15): a, c, g, t, unknown or other
taatatgtagcgagnaagtagatcaaggc

SEQ ID NO: 1076 - DNA, 29 bp, Homo sapiens
  modified_base (15)..(15): a, c, g, t, unknown or other
ctgaaaaagacaatnatataaatcattaa

SEQ ID NO: 1077 - DNA, 29 bp, Homo sapiens
  modified_base (15)..(15): a, c, g, t, unknown or other
acttgcttatgatantacatcagtccatg

SEQ ID NO: 1078 - DNA, 29 bp, Homo sapiens
  modified_base (15)..(15): a, c, g, t, unknown or other
ctatgaatggtaaancttttgagactatg

SEQ ID NO: 1079 - DNA, 29 bp, Homo sapiens
  modified_base (15)..(15): a, c, g, t, unknown or other
ctggtcctaataagngctatgatcctgag

SEQ ID NO: 1080 - DNA, 29 bp, Homo sapiens
  modified_base (15)..(15): a, c, g, t, unknown or other
tgcccactcattttntagatttccactta

SEQ ID NO: 1081 - DNA, 29 bp, Homo sapiens
  modified_base (15)..(15): a, c, g, t, unknown or other
ctatgcttataagtnatggagttttcaac

SEQ ID NO: 1082 - DNA, 29 bp, Homo sapiens
  modified_base (15)..(15): a, c, g, t, unknown or other
acacatcaccataangaatgacagaatta

SEQ ID NO: 1083 - DNA, 29 bp, Homo sapiens
  modified_base (15)..(15): a, c, g, t, unknown or other
gaatatttcagcaanacttaactcctaac

SEQ ID NO: 1084 - DNA, 29 bp, Homo sapiens
  modified_base (15)..(15): a, c, g, t, unknown or other
aacttacatagagtnctacaaccagagca

SEQ ID NO: 1085 - DNA, 29 bp, Homo sapiens
  modified_base (15)..(15): a, c, g, t, unknown or other
aagactagttagagncaatctaggagaga

SEQ ID NO: 1086 - DNA, 29 bp, Homo sapiens
  modified_base (15)..(15): a, c, g, t, unknown or other
ataagcaaacgaatncaaaaaaagtgcag

SEQ ID NO: 1087 - DNA, 29 bp, Homo sapiens
  modified_base (15)..(15): a, c, g, t, unknown or other
gactatagctggtgnaatcattagcaaac

SEQ ID NO: 1088 - DNA, 29 bp, Homo sapiens
  modified_base (15)..(15): a, c, g, t, unknown or other
gctggaacttaaggnaaatgtgtggcagg

SEQ ID NO: 1089 - DNA, 29 bp, Homo sapiens
  modified_base (15)..(15): a, c, g, t, unknown or other
ttcagtgaaactgtntagcatgacaaaca

SEQ ID NO: 1090 - DNA, 29 bp, Homo sapiens
  modified_base (15)..(15): a, c, g, t, unknown or other
ctgttctaccctgangccagcgtcctgct

SEQ ID NO: 1091 - DNA, 29 bp, Homo sapiens
  modified_base (15)..(15): a, c, g, t, unknown or other
ccgtgaggtaaggcnaagggccaagcagt

SEQ ID NO: 1092 - DNA, 29 bp, Homo sapiens
  modified_base (15)..(15): a, c, g, t, unknown or other
ttcttctagtgacanatctcaaacgtcct

SEQ ID NO: 1093 - DNA, 29 bp, Homo sapiens
  modified_base (15)..(15): a, c, g, t, unknown or other
caacagttgatttangtcatgcttttgtt

SEQ ID NO: 1094 - DNA, 29 bp, Homo sapiens
  modified_base (15)..(15): a, c, g, t, unknown or other
ctctcatcccagacnaataggcatgtgag

SEQ ID NO: 1095 - DNA, 29 bp, Homo sapiens
  modified_base (15)..(15): a, c, g, t, unknown or other
ccaatgcctcagacntgctgcaatataaa

SEQ ID NO: 1096 - DNA, 29 bp, Homo sapiens
  modified_base (15)..(15): a, c, g, t, unknown or other
tttcaatgagaaaanagaaatcttggcag

SEQ ID NO: 1097 - DNA, 29 bp, Homo sapiens
  modified_base (15)..(15): a, c, g, t, unknown or other
aagttttagcagtgngaaggctttgaaag

SEQ ID NO: 1098 - DNA, 29 bp, Homo sapiens
  modified_base (15)..(15): a, c, g, t, unknown or other
ccagttgtgattgcngactcttccccctt

SEQ ID NO: 1099 - DNA, 29 bp, Homo sapiens
  modified_base (15)..(15): a, c, g, t, unknown or other
actgcctgcttcttngtatagctggaagc

SEQ ID NO: 1100 - DNA, 29 bp, Homo sapiens
  modified_base (15)..(15): a, c, g, t, unknown or other
agaggacttagacanggttccagtttttg

SEQ ID NO: 1101 - DNA, 29 bp, Homo sapiens
  modified_base (15)..(15): a, c, g, t, unknown or other
acctacaattttttnaaaagtgccactgg

SEQ ID NO: 1102 - DNA, 29 bp, Homo sapiens
  modified_base (15)..(15): a, c, g, t, unknown or other
cataaagatttgacntgagttcatggaat

SEQ ID NO: 1103 - DNA, 29 bp, Homo sapiens
  modified_base (15)..(15): a, c, g, t, unknown or other
ttttaagtccaaaanaaattctgtttaac

SEQ ID NO: 1104 - DNA, 29 bp, Homo sapiens
  modified_base (15)..(15): a, c, g, t, unknown or other
ttttaaaacatctangctttttccttttg

SEQ ID NO: 1105 - DNA, 29 bp, Homo sapiens
  modified_base (15)..(15): a, c, g, t, unknown or other
ttatcctaggattanaggaagactaattt

SEQ ID NO: 1106 - DNA, 29 bp, Homo sapiens
  modified_base (15)..(15): a, c, g, t, unknown or other
atgcagtatatctanttatttggtagaaa

SEQ ID NO: 1107 - DNA, 29 bp, Homo sapiens
  modified_base (15)..(15): a, c, g, t, unknown or other
gtgcctcttgttganaaacattgagaagg

SEQ ID NO: 1108 - DNA, 29 bp, Homo sapiens
  modified_base (15)..(15): a, c, g, t, unknown or other
actagtaaatcactntttaggtgtttaac

SEQ ID NO: 1109 - DNA, 29 bp, Homo sapiens
  modified_base (15)..(15): a, c, g, t, unknown or other
gcatagggtacagantagttgagaaaatg

SEQ ID NO: 1110 - DNA, 29 bp, Homo sapiens
  modified_base (15)..(15): a, c, g, t, unknown or other
aaatttactgcttanaaagagacctaaga

SEQ ID NO: 1111 - DNA, 29 bp, Homo sapiens
  modified_base (15)..(15): a, c, g, t, unknown or other
agctcagaaggctcncttactttgggcat

SEQ ID NO: 1112 - DNA, 29 bp, Homo sapiens
  modified_base (15)..(15): a, c, g, t, unknown or other
tcagtggtgaagacntgcttcctggtcca

SEQ ID NO: 1113 - DNA, 29 bp, Homo sapiens
  modified_base (15)..(15): a, c, g, t, unknown or other
cctgcctgtctttangtaccttgaccctg

SEQ ID NO: 1114 - DNA, 29 bp, Homo sapiens
  modified_base (15)..(15): a, c, g, t, unknown or other
ggtcagctttgctcnaattttgttttcat

SEQ ID NO: 1115 - DNA, 29 bp, Homo sapiens
  modified_base (15)..(15): a, c, g, t, unknown or other
gtttcacaatgaatnatggcagcaactgg

SEQ ID NO: 1116 - DNA, 29 bp, Homo sapiens
  modified_base (15)..(15): a, c, g, t, unknown or other
tatacacacgattgnttttaagactgtta

SEQ ID NO: 1117 - DNA, 29 bp, Homo sapiens
  modified_base (15)..(15): a, c, g, t, unknown or other
tgttacttagataanactgcaagatccat

SEQ ID NO: 1118 - DNA, 29 bp, Homo sapiens
  modified_base (15)..(15): a, c, g, t, unknown or other
acagcatcataattnactgaagacccaaa

SEQ ID NO: 1119 - DNA, 29 bp, Homo sapiens
  modified_base (15)..(15): a, c, g, t, unknown or other
tccatataggctacntgcatactgggtat

SEQ ID NO: 1120 - DNA, 29 bp, Homo sapiens
  modified_base (15)..(15): a, c, g, t, unknown or other
caggccgacactgangcctggaaactacc

SEQ ID NO: 1121 - DNA, 29 bp, Homo sapiens
  modified_base (15)..(15): a, c, g, t, unknown or other
ttacggtcccttttnaaactcagcctgaa

SEQ ID NO: 1122 - DNA, 29 bp, Homo sapiens
  modified_base (15)..(15): a, c, g, t, unknown or other
cttgtttcattgctntggaagttgtgtta

SEQ ID NO: 1123 - DNA, 29 bp, Homo sapiens
  modified_base (15)..(15): a, c, g, t, unknown or other
gtttcagtgattgancttcatttaaagag

SEQ ID NO: 1124 - DNA, 29 bp, Homo sapiens
  modified_base (15)..(15): a, c, g, t, unknown or other
ccagttaccaactgngttctcttctccaa

SEQ ID NO: 1125 - DNA, 29 bp, Homo sapiens
  modified_base (15)..(15): a, c, g, t, unknown or other
cagccaaatttaggnttgataatattatg

SEQ ID NO: 1126 - DNA, 29 bp, Homo sapiens
  modified_base (15)..(15): a, c, g, t, unknown or other
tcctgagatgtccanggagtagcaagaga

SEQ ID NO: 1127 - DNA, 29 bp, Homo sapiens
  modified_base (15)..(15): a, c, g, t, unknown or other
atgataaaacatagnaacttttaaaagat

SEQ ID NO: 1128 - DNA, 29 bp, Homo sapiens
  modified_base (15)..(15): a, c, g, t, unknown or other
gaacctaaagatctntaaagtctctgcaa

SEQ ID NO: 1129 - DNA, 29 bp, Homo sapiens
  modified_base (15)..(15): a, c, g, t, unknown or other
tgaaatggaggtctntgtaatgaacaaga

SEQ ID NO: 1130 - DNA, 29 bp, Homo sapiens
  modified_base (15)..(15): a, c, g, t, unknown or other
gggagacagaaggtnaaccatatgtggag

SEQ ID NO: 1131 - DNA, 29 bp, Homo sapiens
  modified_base (15)..(15): a, c, g, t, unknown or other
ccccttgaccaatgntttggtgattggga

SEQ ID NO: 1132 - DNA, 29 bp, Homo sapiens
  modified_base (15)..(15): a, c, g, t, unknown or other
tattttgtaagtcanagtcaagggcttga

SEQ ID NO: 1133 - DNA, 29 bp, Homo sapiens
  modified_base (15)..(15): a, c, g, t, unknown or other
tcctatggatgcggnattctctattcctg

SEQ ID NO: 1134 - DNA, 29 bp, Homo sapiens
  modified_base (15)..(15): a, c, g, t, unknown or other
aaaactctcacactncattctatgatgga

SEQ ID NO: 1135 - DNA, 29 bp, Homo sapiens
  modified_base (15)..(15): a, c, g, t, unknown or other
aaatgtcatcctacnctatttcctctgag

SEQ ID NO: 1136 - DNA, 29 bp, Homo sapiens
  modified_base (15)..(15): a, c, g, t, unknown or other
ctgcaacactgtggnttcaccaacaccta

SEQ ID NO: 1137 - DNA, 29 bp, Homo sapiens
  modified_base (15)..(15): a, c, g, t, unknown or other
aagatttgtccatgnaaacctgtcccatt

SEQ ID NO: 1138 - DNA, 29 bp, Homo sapiens
  modified_base (15)..(15): a, c, g, t, unknown or other
tatcaaaacctaagntaagtgacttcttg

SEQ ID NO: 1139 - DNA, 29 bp, Homo sapiens
  modified_base (15)..(15): a, c, g, t, unknown or other
atttcagccaccggnccagcattgctctt

SEQ ID NO: 1140 - DNA, 29 bp, Homo sapiens
  modified_base (15)..(15): a, c, g, t, unknown or other
ataaatcaggaggantaaatgaagttaag

SEQ ID NO: 1141 - DNA, 29 bp, Homo sapiens
  modified_base (15)..(15): a, c, g, t, unknown or other
ccggttgtatcttantgtgtgacttgtaa

SEQ ID NO: 1142 - DNA, 29 bp, Homo sapiens
  modified_base (15)..(15): a, c, g, t, unknown or other
tctgtagtatattcnccaactttacacat

SEQ ID NO: 1143 - DNA, 29 bp, Homo sapiens
  modified_base (15)..(15): a, c, g, t, unknown or other
tatgttgggcggtcngccagagaactatc

SEQ ID NO: 1144 - DNA, 29 bp, Homo sapiens
  modified_base (15)..(15): a, c, g, t, unknown or other
gtgtccttaacattnaggaatgacatagc

SEQ ID NO: 1145 - DNA, 29 bp, Homo sapiens
  modified_base (15)..(15): a, c, g, t, unknown or other
ctacctttctttganaatgtgatgctgtg

SEQ ID NO: 1146 - DNA, 29 bp, Homo sapiens
  modified_base (15)..(15): a, c, g, t, unknown or other
tctattgtgctgtangccagaaccaattc

SEQ ID NO: 1147 - DNA, 29 bp, Homo sapiens
  modified_base (15)..(15): a, c, g, t, unknown or other
ctcagatttttaatngagtaatggtagat

SEQ ID NO: 1148 - DNA, 29 bp, Homo sapiens
  modified_base (15)..(15): a, c, g, t, unknown or other
agcactataggattngagtctcccgatgg

SEQ ID NO: 1149 - DNA, 29 bp, Homo sapiens
  modified_base (15)..(15): a, c, g, t, unknown or other
tgatcacgaacgggntttgtttgttgcac

SEQ ID NO: 1150 - DNA, 29 bp, Homo sapiens
  modified_base (15)..(15): a, c, g, t, unknown or other
tggttttagtgccangatggaatcagggc

SEQ ID NO: 1151 - DNA, 29 bp, Homo sapiens
  modified_base (15)..(15): a, c, g, t, unknown or other
ctggtcaacctcatngcccaaatcccttt

SEQ ID NO: 1152 - DNA, 29 bp, Homo sapiens
  modified_base (15)..(15): a, c, g, t, unknown or other
tatgattaaataccnaaatatgtacttct

SEQ ID NO: 1153 - DNA, 29 bp, Homo sapiens
  modified_base (15)..(15): a, c, g, t, unknown or other
tttgaacaacttagnttaagccgttcttt

SEQ ID NO: 1154 - DNA, 29 bp, Homo sapiens
  modified_base (15)..(15): a, c, g, t, unknown or other
gtatttgagatcacnttgtaaagctagtt

SEQ ID NO: 1155 - DNA, 29 bp, Homo sapiens
  modified_base (15)..(15): a, c, g, t, unknown or other
cctattagatgcaantataccgacctcca

SEQ ID NO: 1156 - DNA, 29 bp, Homo sapiens
  modified_base (15)..(15): a, c, g, t, unknown or other
gtgatgacattaacnatgaatttacctca

SEQ ID NO: 1157 - DNA, 29 bp, Homo sapiens
  modified_base (15)..(15): a, c, g, t, unknown or other
tacctaattcagggntccatagctgagaa

SEQ ID NO: 1158 - DNA, 29 bp, Homo sapiens
  modified_base (15)..(15): a, c, g, t, unknown or other
ttgtagaaatacccnttgcctaccaatat

SEQ ID NO: 1159 - DNA, 29 bp, Homo sapiens
  modified_base (15)..(15): a, c, g, t, unknown or other
aatcctccagcattncagtcatgagacaa

SEQ ID NO: 1160 - DNA, 29 bp, Homo sapiens
  modified_base (15)..(15): a, c, g, t, unknown or other
tttacaaaacattcntgtacttccaaagg

SEQ ID NO: 1161 - DNA, 29 bp, Homo sapiens
  modified_base (15)..(15): a, c, g, t, unknown or other
ttgccgatactatcntcaagcctttccca

SEQ ID NO: 1162 - DNA, 29 bp, Homo sapiens
  modified_base (15)..(15): a, c, g, t, unknown or other
atcacagagccccgnagcaagcgctgaac

SEQ ID NO: 1163 - DNA, 29 bp, Homo sapiens
  modified_base (15)..(15): a, c, g, t, unknown or other
gattttaccactcanagcatcaggtatgc

SEQ ID NO: 1164 - DNA, 29 bp, Homo sapiens
  modified_base (15)..(15): a, c, g, t, unknown or other
ttctgggcagaatanttaaccgatactgt

SEQ ID NO: 1165 - DNA, 29 bp, Homo sapiens
  modified_base (15)..(15): a, c, g, t, unknown or other
aaaagttctgtatcnatgttgagtttgtt

SEQ ID NO: 1166 - DNA, 29 bp, Homo sapiens
  modified_base (15)..(15): a, c, g, t, unknown or other
tatgagaacatttangcatttttctctta

SEQ ID NO: 1167 - DNA, 29 bp, Homo sapiens
  modified_base (15)..(15): a, c, g, t, unknown or other
cattaatcaatctgnaattcctttcaccc

SEQ ID NO: 1168 - DNA, 29 bp, Homo sapiens
  modified_base (15)..(15): a, c, g, t, unknown or other
ctgaacctttaatangattcaatagctct

SEQ ID NO: 1169 - DNA, 29 bp, Homo sapiens
  modified_base (15)..(15): a, c, g, t, unknown or other
aatgacctccttctnttgggttagcagaa

SEQ ID NO: 1170 - DNA, 29 bp, Homo sapiens
  modified_base (15)..(15): a, c, g, t, unknown or other
ttacctcgtcaaacnttcacatgtatcac

SEQ ID NO: 1171 - DNA, 29 bp, Homo sapiens
  modified_base (15)..(15): a, c, g, t, unknown or other
agactatctttattnataactgtggactt

SEQ ID NO: 1172 - DNA, 29 bp, Homo sapiens
  modified_base (15)..(15): a, c, g, t, unknown or other
acaaatgaaataccntggagacacgattt

SEQ ID NO: 1173 - DNA, 29 bp, Homo sapiens
  modified_base (15)..(15): a, c, g, t, unknown or other
acctgggacagagcngccctgttattcta

SEQ ID NO: 1174 - DNA, 29 bp, Homo sapiens
  modified_base (15)..(15): a, c, g, t, unknown or other
aagtacctacttgtnttagagaagtgtgt

SEQ ID NO: 1175 - DNA, 29 bp, Homo sapiens
  modified_base (15)..(15): a, c, g, t, unknown or other
aaataaaactgacangagccttaccctct

SEQ ID NO: 1176 - DNA, 29 bp, Homo sapiens
  modified_base (15)..(15): a, c, g, t, unknown or other
ggagttgaaactgcnaaataaatttgtag

SEQ ID NO: 1177 - DNA, 29 bp, Homo sapiens
  modified_base (15)..(15): a, c, g, t, unknown or other
caaacaaataaaggntttggcaagtcaag

SEQ ID NO: 1178 - DNA, 29 bp, Homo sapiens
  modified_base (15)..(15): a, c, g, t, unknown or other
tacaactggccaganactgtgctgctata

SEQ ID NO: 1179 - DNA, 29 bp, Homo sapiens
  modified_base (15)..(15): a, c, g, t, unknown or other
caccgaacagactgncagcccaaatgtta

SEQ ID NO: 1180 - DNA, 29 bp, Homo sapiens
  modified_base (15)..(15): a, c, g, t, unknown or other
aaacgttgcccttanatacagctgcactg

SEQ ID NO: 1181 - DNA, 29 bp, Homo sapiens
  modified_base (15)..(15): a, c, g, t, unknown or other
ttaaatgcttgaacncagccattatgcac

SEQ ID NO: 1182 - DNA, 29 bp, Homo sapiens
  modified_base (15)..(15): a, c, g, t, unknown or other
cacagtgggagcctntctgtggtatcccc

SEQ ID NO: 1183 - DNA, 29 bp, Homo sapiens
  modified_base (15)..(15): a, c, g, t, unknown or other
tttgggcctgccacntctatacggagcta

SEQ ID NO: 1184 - DNA, 29 bp, Homo sapiens
  modified_base (15)..(15): a, c, g, t, unknown or other
gtcacgctgggatcncgaatagtttccca

SEQ ID NO: 1185 - DNA, 29 bp, Homo sapiens
  modified_base (15)..(15): a, c, g, t, unknown or other
cctttataatattcntcttttgcttaagt

SEQ ID NO: 1186 - DNA, 29 bp, Homo sapiens
  modified_base (15)..(15): a, c, g, t, unknown or other
gaactggtactgacnggctccaacttcct

SEQ ID NO: 1187 - DNA, 29 bp, Homo sapiens
  modified_base (15)..(15): a, c, g, t, unknown or other
ttaaccaaaggttcngggattcatgctcc

SEQ ID NO: 1188 - DNA, 29 bp, Homo sapiens
  modified_base (15)..(15): a, c, g, t, unknown or other
actgttttttctgantggcaaacctaaga

SEQ ID NO: 1189 - DNA, 29 bp, Homo sapiens
  modified_base (15)..(15): a, c, g, t, unknown or other
tatcagattcacccnccagttatttgtaa

SEQ ID NO: 1190 - DNA, 29 bp, Homo sapiens
  modified_base (15)..(15): a, c, g, t, unknown or other
gtcaggaggaacccntaagtgtctctcat

SEQ ID NO: 1191 - DNA, 29 bp, Homo sapiens
  modified_base (15)..(15): a, c, g, t, unknown or other
aagagccaaaagagnacatgacggccctg

SEQ ID NO: 1192 - DNA, 29 bp, Homo sapiens
  modified_base (15)..(15): a, c, g, t, unknown or other
ttgtgggagcaggcngtagttatctcgag

SEQ ID NO: 1193 - DNA, 29 bp, Homo sapiens
  modified_base (15)..(15): a, c, g, t, unknown or other
cactttcctaattcnagcacttctaggta

SEQ ID NO: 1194 - DNA, 29 bp, Homo sapiens
  modified_base (15)..(15): a, c, g, t, unknown or other
aatgttactgagtanaaaggacaacatca

SEQ ID NO: 1195 - DNA, 29 bp, Homo sapiens
  modified_base (15)..(15): a, c, g, t, unknown or other
agtttctgtaatcancacccttaattcac

SEQ ID NO: 1196 - DNA, 29 bp, Homo sapiens
  modified_base (15)..(15): a, c, g, t, unknown or other
ttgttttgtcttctnttatttaccaaccc

SEQ ID NO: 1197 - DNA, 29 bp, Homo sapiens
  modified_base (15)..(15): a, c, g, t, unknown or other
accattgccataganattgttttttctaa

SEQ ID NO: 1198 - DNA, 29 bp, Homo sapiens
  modified_base (15)..(15): a, c, g, t, unknown or other
acctctgtcagacangaggagctacattt

SEQ ID NO: 1199 - DNA, 29 bp, Homo sapiens
  modified_base (15)..(15): a, c, g, t, unknown or other
attgttggttttccntgagtggtacctgg

SEQ ID NO: 1200 - DNA, 29 bp, Homo sapiens
  modified_base (15)..(15): a, c, g, t, unknown or other
atattgtggtagttnatgaacataattcc

SEQ ID NO: 1201 - DNA, 29 bp, Homo sapiens
  modified_base (15)..(15): a, c, g, t, unknown or other
caagtgttttaatanatgcgctaattgag

SEQ ID NO: 1202 - DNA, 29 bp, Homo sapiens
  modified_base (15)..(15): a, c, g, t, unknown or other
tttctgattttcagnccaagaagttttgt

SEQ ID NO: 1203 - DNA, 29 bp, Homo sapiens
  modified_base (15)..(15): a, c, g, t, unknown or other
gtttatttgcagacntaaaaggcagttcc

SEQ ID NO: 1204 - DNA, 29 bp, Homo sapiens
  modified_base (15)..(15): a, c, g, t, unknown or other
gaaccaaatcattgnggttgttgaggtag

SEQ ID NO: 1205 - DNA, 29 bp, Homo sapiens
  modified_base (15)..(15): a, c, g, t, unknown or other
aagactgctagattnttttgaaccagatg

SEQ ID NO: 1206 - DNA, 29 bp, Homo sapiens
  modified_base (15)..(15): a, c, g, t, unknown or other
ataaatgacagtcanttcaaaaatcaggg

SEQ ID NO: 1207 - DNA, 29 bp, Homo sapiens
  modified_base (15)..(15): a, c, g, t, unknown or other
aaatcccctgctccntgttatgtcttttc

SEQ ID NO: 1208 - DNA, 29 bp, Homo sapiens
  modified_base (15)..(15): a, c, g, t, unknown or other
tttgtacgtgtgtangggtgagcaggtca

SEQ ID NO: 1209 - DNA, 29 bp, Homo sapiens
  modified_base (15)..(15): a, c, g, t, unknown or other
agctctatttttcantctagcccaaatac

SEQ ID NO: 1210 - DNA, 29 bp, Homo sapiens
  modified_base (15)..(15): a, c, g, t, unknown or other
gtgctgtcaccgtcnggcccgctgtcccc

SEQ ID NO: 1211 - DNA, 29 bp, Homo sapiens
  modified_base (15)..(15): a, c, g, t, unknown or other
ttcaaatggattcanaatcaatgataaca

SEQ ID NO: 1212 - DNA, 29 bp, Homo sapiens
  modified_base (15)..(15): a, c, g, t, unknown or other
cctaaggcaagaccnctaatttagctgtg

SEQ ID NO: 1213 - DNA, 29 bp, Homo sapiens
  modified_base (15)..(15): a, c, g, t, unknown or other
gaccagcttgggctncgtgcgttctgcct

SEQ ID NO: 1214 - DNA, 29 bp, Homo sapiens
  modified_base (15)..(15): a, c, g, t, unknown or other
catctttccctttcncttgggtaaatacc

SEQ ID NO: 1215 - DNA, 29 bp, Homo sapiens
  modified_base (15)..(15): a, c, g, t, unknown or other
atttatgttcctctntatactagccattc

SEQ ID NO: 1216 - DNA, 29 bp, Homo sapiens
  modified_base (15)..(15): a, c, g, t, unknown or other
taaactaatttgganatttggagggctgc

SEQ ID NO: 1217 - DNA, 29 bp, Homo sapiens
  modified_base (15)..(15): a, c, g, t, unknown or other
caagcacaaagtacnggccaggggggtct

SEQ ID NO: 1218 - DNA, 29 bp, Homo sapiens
  modified_base (15)..(15): a, c, g, t, unknown or other
agtagtttgtggagngatgtttccatctc

SEQ ID NO: 1219 - DNA, 29 bp, Homo sapiens
  modified_base (15)..(15): a, c, g, t, unknown or other
gaaagcctgctggcncaactctttacagg

SEQ ID NO: 1220 - DNA, 29 bp, Homo sapiens
  modified_base (15)..(15): a, c, g, t, unknown or other
aatccttggcataangtttaagatctttc

SEQ ID NO: 1221 - DNA, 29 bp, Homo sapiens
  modified_base (15)..(15): a, c, g, t, unknown or other
aatcagtcatagatntaaagctgcagtaa

SEQ ID NO: 1222 - DNA, 29 bp, Homo sapiens
  modified_base (15)..(15): a, c, g, t, unknown or other
taagttttatatttnactaaggttgaata

SEQ ID NO: 1223 - DNA, 29 bp, Homo sapiens
  modified_base (15)..(15): a, c, g, t, unknown or other
gatgaggtgggaaanaaagaggagtccct

SEQ ID NO: 1224 - DNA, 29 bp, Homo sapiens
  modified_base (15)..(15): a, c, g, t, unknown or other
ttgggtgatcagctntttggacatagagt

SEQ ID NO: 1225 - DNA, 29 bp, Homo sapiens
  modified_base (15)..(15): a, c, g, t, unknown or other
cactaacgagaaagntaattcacctacag

SEQ ID NO: 1226 - DNA, 29 bp, Homo sapiens
  modified_base (15)..(15): a, c, g, t, unknown or other
tgaaagatatctagnaccattgctggtac

SEQ ID NO: 1227 - DNA, 29 bp, Homo sapiens
  modified_base (15)..(15): a, c, g, t, unknown or other
aggtacttataatantttgcagggtatct

SEQ ID NO: 1228 - DNA, 29 bp, Homo sapiens
  modified_base (15)..(15): a, c, g, t, unknown or other
ctaaatgctaagagnagagtgtagtttgt

SEQ ID NO: 1229 - DNA, 29 bp, Homo sapiens
  modified_base (15)..(15): a, c, g, t, unknown or other
tttatattgttacanttaagtgttcttgc

SEQ ID NO: 1230 - DNA, 29 bp, Homo sapiens
  modified_base (15)..(15): a, c, g, t, unknown or other
gaacctcaggacaangtctacacacacac

SEQ ID NO: 1231 - DNA, 29 bp, Homo sapiens
  modified_base (15)..(15): a, c, g, t, unknown or other
gttcacatgcttttntacacatgtgcatt

SEQ ID NO: 1232 - DNA, 29 bp, Homo sapiens
  modified_base (15)..(15): a, c, g, t, unknown or other
gcactaaccctgaanagggataggcgccc

SEQ ID NO: 1233 - DNA, 29 bp, Homo sapiens
  modified_base (15)..(15): a, c, g, t, unknown or other
caatagggcttttangatatcagaaaagt

SEQ ID NO: 1234 - DNA, 29 bp, Homo sapiens
  modified_base (15)..(15): a, c, g, t, unknown or other
gtaactcgatcttcnacagattaagtgac

SEQ ID NO: 1235 - DNA, 29 bp, Homo sapiens
  modified_base (15)..(15): a, c, g, t, unknown or other
tctgtctttctgttngttggtggctaatg

SEQ ID NO: 1236 - DNA, 29 bp, Homo sapiens
  modified_base (15)..(15): a, c, g, t, unknown or other
tctttgcagccaggngaacctttagctaa

SEQ ID NO: 1237 - DNA, 29 bp, Homo sapiens
  modified_base (15)..(15): a, c, g, t, unknown or other
caacatgttgtggtngggcctagttggtt

SEQ ID NO: 1238 - DNA, 29 bp, Homo sapiens
  modified_base (15)..(15): a, c, g, t, unknown or other
ccacacatcctgccntgaagtactacctt

SEQ ID NO: 1239 - DNA, 29 bp, Homo sapiens
  modified_base (15)..(15): a, c, g, t, unknown or other
ttcaccctgcaaagngtgatatgtctgaa

SEQ ID NO: 1240 - DNA, 29 bp, Homo sapiens
  modified_base (15)..(15): a, c, g, t, unknown or other
ttcctaccgttatcntgactgtagagtgg

SEQ ID NO: 1241 - DNA, 29 bp, Homo sapiens
  modified_base (15)..(15): a, c, g, t, unknown or other
ggacctgctgctcanaggacatttcagag

SEQ ID NO: 1242 - DNA, 29 bp, Homo sapiens
  modified_base (15)..(15): a, c, g, t, unknown or other
aagttgccgtctatnagatacgtatttta

SEQ ID NO: 1243 - DNA, 29 bp, Homo sapiens
  modified_base (15)..(15): a, c, g, t, unknown or other
ttggctgagatccangcttcttggagccc

SEQ ID NO: 1244 - DNA, 29 bp, Homo sapiens
  modified_base (15)..(15): a, c, g, t, unknown or other
aactgtggctctctntcctgaattccatg

SEQ ID NO: 1245 - DNA, 29 bp, Homo sapiens
  modified_base (15)..(15): a, c, g, t, unknown or other
caaaaggagcccttngagaggttggctca

SEQ ID NO: 1246 - DNA, 29 bp, Homo sapiens
  modified_base (15)..(15): a, c, g, t, unknown or other
acagggaacctgagngcaggatggtaagg

SEQ ID NO: 1247 - DNA, 29 bp, Homo sapiens
  modified_base (15)..(15): a, c, g, t, unknown or other
ccctgccattaccgnggacggccctcact

SEQ ID NO: 1248 - DNA, 29 bp, Homo sapiens
  modified_base (15)..(15): a, c, g, t, unknown or other
atattttaggatacngactggattttgga

SEQ ID NO: 1249 - DNA, 29 bp, Homo sapiens
  modified_base (15)..(15): a, c, g, t, unknown or other
tatgctgtatatcancaatggattaaaga

SEQ ID NO: 1250 - DNA, 29 bp, Homo sapiens
  modified_base (15)..(15): a, c, g, t, unknown or other
gagaagagtgtgacnagtgtcatttgcac

SEQ ID NO: 1251 - DNA, 29 bp, Homo sapiens
  modified_base (15)..(15): a, c, g, t, unknown or other
tgtgagggcagctgngtctctggctgtgt

SEQ ID NO: 1252 - DNA, 29 bp, Homo sapiens
  modified_base (15)..(15): a, c, g, t, unknown or other
ggcttgtatagtttncgcttcttgggatt

SEQ ID NO: 1253 - DNA, 29 bp, Homo sapiens
  modified_base (15)..(15): a, c, g, t, unknown or other
gcatcatactgcggngatttgacccgagt

SEQ ID NO: 1254 - DNA, 29 bp, Homo sapiens
  modified_base (15)..(15): a, c, g, t, unknown or other
tcttcagtttgttanaatccttacaactt

SEQ ID NO: 1255 - DNA, 29 bp, Homo sapiens
  modified_base (15)..(15): a, c, g, t, unknown or other
tagcagatgaattancagatgatgaggtc

SEQ ID NO: 1256 - DNA, 29 bp, Homo sapiens
  modified_base (15)..(15): a, c, g, t, unknown or other
acatttcaaccatcnagaaccactgaact

SEQ ID NO: 1257 - DNA, 29 bp, Homo sapiens
  modified_base (15)..(15): a, c, g, t, unknown or other
ttagggcttttccancattccactttcct

SEQ ID NO: 1258 - DNA, 29 bp, Homo sapiens
  modified_base (15)..(15): a, c, g, t, unknown or other
aagttctcatgtgancctacccgcctgat

SEQ ID NO: 1259 - DNA, 29 bp, Homo sapiens
  modified_base (15)..(15): a, c, g, t, unknown or other
acacttcattatagnttggatcatattta

SEQ ID NO: 1260 - DNA, 29 bp, Homo sapiens
  modified_base (15)..(15): a, c, g, t, unknown or other
aaagtattatgcacntttatcattgcagg

SEQ ID NO: 1261 - DNA, 29 bp, Homo sapiens
  modified_base (15)..(15): a, c, g, t, unknown or other
ttgatttagaggctnataggtccagctaa

SEQ ID NO: 1262 - DNA, 29 bp, Homo sapiens
  modified_base (15)..(15): a, c, g, t, unknown or other
caatatgaacataancaaaattactcctc

SEQ ID NO: 1263 - DNA, 29 bp, Homo sapiens
  modified_base (15)..(15): a, c, g, t, unknown or other
cagtccttggtcatnagactgaatgagtt

SEQ ID NO: 1264 - DNA, 29 bp, Homo sapiens
  modified_base (15)..(15): a, c, g, t, unknown or other
tagagacaaaaaatnagaagtcagtgact

SEQ ID NO: 1265 - DNA, 29 bp, Homo sapiens
  modified_base (15)..(15): a, c, g, t, unknown or other
aggtgttactaactngatcattcttctgg

SEQ ID NO: 1266 - DNA, 29 bp, Homo sapiens
  modified_base (15)..(15): a, c, g, t, unknown or other
tagctgtcaaaatcnagatttttttaaga

SEQ ID NO: 1267 - DNA, 29 bp, Homo sapiens
  modified_base (15)..(15): a, c, g, t, unknown or other
tgtgcattaactgcntagacagctgtgga

SEQ ID NO: 1268 - DNA, 29 bp, Homo sapiens
  modified_base (15)..(15): a, c, g, t, unknown or other
agggaatgagagagnaaattctagtcaaa

SEQ ID NO: 1269 - DNA, 29 bp, Homo sapiens
  modified_base (15)..(15): a, c, g, t, unknown or other
tcactagacactttnaattactgtctata

SEQ ID NO: 1270 - DNA, 29 bp, Homo sapiens
  modified_base (15)..(15): a, c, g, t, unknown or other
ttcacattaacatantctacttagctcat

SEQ ID NO: 1271 - DNA, 29 bp, Homo sapiens
  modified_base (15)..(15): a, c, g, t, unknown or other
catacctttccagtntattcagtaaatgc

SEQ ID NO: 1272 - DNA, 29 bp, Homo sapiens
  modified_base (15)..(15): a, c, g, t, unknown or other
acatcagtgcttagnatataaagtatgtg

SEQ ID NO: 1273 - DNA, 29 bp, Homo sapiens
  modified_base (15)..(15): a, c, g, t, unknown or other
acttgactgtgggancaccattccctgca

SEQ ID NO: 1274 - DNA, 29 bp, Homo sapiens
  modified_base (15)..(15): a, c, g, t, unknown or other
tccctttttaatgantacccacacagagc

SEQ ID NO: 1275 - DNA, 29 bp, Homo sapiens
  modified_base (15)..(15): a, c, g, t, unknown or other
gagaagaaagatgtntgcttttctgttat

SEQ ID NO: 1276 - DNA, 29 bp, Homo sapiens
  modified_base (15)..(15): a, c, g, t, unknown or other
ttgctgcttagacanggttttttttattg

SEQ ID NO: 1277 - DNA, 29 bp, Homo sapiens
  modified_base (15)..(15): a, c, g, t, unknown or other
aattattctggtaanattggtatgaagtg

SEQ ID NO: 1278 - DNA, 29 bp, Homo sapiens
  modified_base (15)..(15): a, c, g, t, unknown or other
tcagtgacgctctancatagcttgttata

SEQ ID NO: 1279 - DNA, 29 bp, Homo sapiens
  modified_base (15)..(15): a, c, g, t, unknown or other
tctcatgaataattncagaactaggatta

SEQ ID NO: 1280 - DNA, 29 bp, Homo sapiens
  modified_base (15)..(15): a, c, g, t, unknown or other
atacatttgacaacnatatgacgtaccat

SEQ ID NO: 1281 - DNA, 29 bp, Homo sapiens
  modified_base (15)..(15): a, c, g, t, unknown or other
tgctgtctctttctntttcactggttgac

SEQ ID NO: 1282 - DNA, 29 bp, Homo sapiens
  modified_base (15)..(15): a, c, g, t, unknown or other
aaggaacgttagganggacctatctggaa

SEQ ID NO: 1283 - DNA, 29 bp, Homo sapiens
  modified_base (15)..(15): a, c, g, t, unknown or other
gttatattatcaaanacattgaagtttta

SEQ ID NO: 1284 - DNA, 29 bp, Homo sapiens
  modified_base (15)..(15): a, c, g, t, unknown or other
gtagagaacagtgangatattctagagtt

SEQ ID NO: 1285 - DNA, 29 bp, Homo sapiens
  modified_base (15)..(15): a, c, g, t, unknown or other
agttggtatgcttcnaagtgattagctca

SEQ ID NO: 1286 - DNA, 29 bp, Homo sapiens
  modified_base (15)..(15): a, c, g, t, unknown or other
tattgaatgcttaangttaatggctgtga

SEQ ID NO: 1287 - DNA, 29 bp, Homo sapiens
  modified_base (15)..(15): a, c, g, t, unknown or other
gagattcacaagacntcaacactgccttc

SEQ ID NO: 1288 - DNA, 29 bp, Homo sapiens
  modified_base (15)..(15): a, c, g, t, unknown or other
tgttgactttggtcntcactcctttaggc

SEQ ID NO: 1289 - DNA, 29 bp, Homo sapiens
  modified_base (15)..(15): a, c, g, t, unknown or other
tgcagatgccctganctttggaatgaatt

SEQ ID NO: 1290 - DNA, 29 bp, Homo sapiens
  modified_base (15)..(15): a, c, g, t, unknown or other
aataagaacaatttntccatgacagcttt

SEQ ID NO: 1291 - DNA, 29 bp, Homo sapiens
  modified_base (15)..(15): a, c, g, t, unknown or other
caggtttagatcacntcgctaatgttctt

SEQ ID NO: 1292 - DNA, 29 bp, Homo sapiens
  modified_base (15)..(15): a, c, g, t, unknown or other
ctttgtgtggtgatngtacactctctgca

SEQ ID NO: 1293 - DNA, 29 bp, Homo sapiens
  modified_base (15)..(15): a, c, g, t, unknown or other
gcaaagtgcataganggatttattaattt

SEQ ID NO: 1294 - DNA, 29 bp, Homo sapiens
  modified_base (15)..(15): a, c, g, t, unknown or other
actggcaattcttgntccccattgttcta

SEQ ID NO: 1295 - DNA, 29 bp, Homo sapiens
  modified_base (15)..(15): a, c, g, t, unknown or other
gttgcagcctggccntttacgttctggcc

SEQ ID NO: 1296 - DNA, 29 bp, Homo sapiens
  modified_base (15)..(15): a, c, g, t, unknown or other
caataactttatccngctgatgagcacat

SEQ ID NO: 1297 - DNA, 29 bp, Homo sapiens
  modified_base (15)..(15): a, c, g, t, unknown or other
ggtttggcttcagcngtccctcagaccta

SEQ ID NO: 1298 - DNA, 29 bp, Homo sapiens
  modified_base (15)..(15): a, c, g, t, unknown or other
ctagcaatccaagcnttagacccagtgga

SEQ ID NO: 1299 - DNA, 29 bp, Homo sapiens
  modified_base (15)..(15): a, c, g, t, unknown or other
acccttttgctgagnatatttggtggtgt

SEQ ID NO: 1300 - DNA, 29 bp, Homo sapiens
  modified_base (15)..(15): a, c, g, t, unknown or other
ccactgcttttaggntcaaatataaacct

SEQ ID NO: 1301 - DNA, 29 bp, Homo sapiens
  modified_base (15)..(15): a, c, g, t, unknown or other
cactcacactacagnagagttggatactg

SEQ ID NO: 1302 - DNA, 29 bp, Homo sapiens
  modified_base (15)..(15): a, c, g, t, unknown or other
gtgactggctgaacntggatcaattgctc

SEQ ID NO: 1303 - DNA, 29 bp, Homo sapiens
  modified_base (15)..(15): a, c, g, t, unknown or other
aactagagataccangtattttgttactg

SEQ ID NO: 1304 - DNA, 29 bp, Homo sapiens
  modified_base (15)..(15): a, c, g, t, unknown or other
agatcaatggtcacnaaacgttttattta

SEQ ID NO: 1305 - DNA, 29 bp, Homo sapiens
  modified_base (15)..(15): a, c, g, t, unknown or other
agtaacaccaatatntgtagaaccattct

SEQ ID NO: 1306 - DNA, 29 bp, Homo sapiens
  modified_base (15)..(15): a, c, g, t, unknown or other
caagggctcagaacngtagctatctgatc

SEQ ID NO: 1307 - DNA, 29 bp, Homo sapiens
  modified_base (15)..(15): a, c, g, t, unknown or other
gaagtttagatagcnagaatcatgcattg

SEQ ID NO: 1308 - DNA, 29 bp, Homo sapiens
  modified_base (15)..(15): a, c, g, t, unknown or other
cctgtgtgtctctgnggctaaatataaac

SEQ ID NO: 1309 - DNA, 29 bp, Homo sapiens
  modified_base (15)..(15): a, c, g, t, unknown or other
agatagtggccccanagaagcagggtaga

SEQ ID NO: 1310 - DNA, 29 bp, Homo sapiens
  modified_base (15)..(15): a, c, g, t, unknown or other
acttttgatgtcccnacagatttagcagc

SEQ ID NO: 1311 - DNA, 29 bp, Homo sapiens
  modified_base (15)..(15): a, c, g, t, unknown or other
ttatcaggtattgcnatgctctttgttgc

SEQ ID NO: 1312 - DNA, 29 bp, Homo sapiens
  modified_base (15)..(15): a, c, g, t, unknown or other
tatgcccctcagtangtcacacacactca

SEQ ID NO: 1313 - DNA, 29 bp, Homo sapiens
  modified_base (15)..(15): a, c, g, t, unknown or other
ttgagatggtcctgnccaaagagtcttca

SEQ ID NO: 1314 - DNA, 29 bp, Homo sapiens
  modified_base (15)..(15): a, c, g, t, unknown or other
atctttgaactttcnagaaatggttctag

SEQ ID NO: 1315 - DNA, 29 bp, Homo sapiens
  modified_base (15)..(15): a, c, g, t, unknown or other
ttctgagtttatgtngtaaattgtaattt

SEQ ID NO: 1316 - DNA, 29 bp, Homo sapiens
  modified_base (15)..(15): a, c, g, t, unknown or other
tgcacatttgctggntctatagatgtaga

SEQ ID NO: 1317 - DNA, 29 bp, Homo sapiens
  modified_base (15)..(15): a, c, g, t, unknown or other
gaaaatggagacacngaaacacatcagag

SEQ ID NO: 1318 - DNA, 29 bp, Homo sapiens
  modified_base (15)..(15): a, c, g, t, unknown or other
aataggtcaagaatngaatcgtgtgaatg

SEQ ID NO: 1319 - DNA, 29 bp, Homo sapiens
  modified_base (15)..(15): a, c, g, t, unknown or other
agccattttctgccncgattgtggatttg

SEQ ID NO: 1320 - DNA, 29 bp, Homo sapiens
  modified_base (15)..(15): a, c, g, t, unknown or other
tatagtaaatttacngtaggtttagacgt

SEQ ID NO: 1321 - DNA, 29 bp, Homo sapiens
  modified_base (15)..(15): a, c, g, t, unknown or other
tctgccacataatanatactcaataaagt

SEQ ID NO: 1322 - DNA, 29 bp, Homo sapiens
  modified_base (15)..(15): a, c, g, t, unknown or other
taggcaaggttttgngggctaagcctgag

SEQ ID NO: 1323 - DNA, 29 bp, Homo sapiens
  modified_base (15)..(15): a, c, g, t, unknown or other
ctgaactagtaatgnataagtaggagaaa

SEQ ID NO: 1324 - DNA, 29 bp, Homo sapiens
  modified_base (15)..(15): a, c, g, t, unknown or other
aagcttaataatttntcaaagttcaacta

SEQ ID NO: 1325 - DNA, 29 bp, Homo sapiens
  modified_base (15)..(15): a, c, g, t, unknown or other
aggtggcccaaggtngattgtgaacactc

SEQ ID NO: 1326 - DNA, 29 bp, Homo sapiens
  modified_base (15)..(15): a, c, g, t, unknown or other
tttagtcacatgganataaaagttgacct

SEQ ID NO: 1327 - DNA, 29 bp, Homo sapiens
  modified_base (15)..(15): a, c, g, t, unknown or other
aagactatgatgatnaatggaattattac

SEQ ID NO: 1328 - DNA, 29 bp, Homo sapiens
  modified_base (15)..(15): a, c, g, t, unknown or other
ccaaccatgtcttancttagtgtccttag

SEQ ID NO: 1329 - DNA, 29 bp, Homo sapiens
  modified_base (15)..(15): a, c, g, t, unknown or other
ttatcttatggttgnctactattcataaa

SEQ ID NO: 1330 - DNA, 29 bp, Homo sapiens
  modified_base (15)..(15): a, c, g, t, unknown or other
ctcagagaccctcanctgtatctttcagg

SEQ ID NO: 1331 - DNA, 29 bp, Homo sapiens
  modified_base (15)..(15): a, c, g, t, unknown or other
ttacctatggcagcnacacctctgatagc

SEQ ID NO: 1332 - DNA, 29 bp, Homo sapiens
  modified_base (15)..(15): a, c, g, t, unknown or other
atacaaagtgatacntaatacactagatg

SEQ ID NO: 1333 - DNA, 29 bp, Homo sapiens
  modified_base (15)..(15): a, c, g, t, unknown or other
actcaggctccatanatagaacactaagc

SEQ ID NO: 1334 - DNA, 29 bp, Homo sapiens
  modified_base (15)..(15): a, c, g, t, unknown or other
aaaaaaatgttttcnggtgttataaataa

SEQ ID NO: 1335 - DNA, 29 bp, Homo sapiens
  modified_base (15)..(15): a, c, g, t, unknown or other
aaggaacacattcantgagtactttactt

SEQ ID NO: 1336 - DNA, 29 bp, Homo sapiens
  modified_base (15)..(15): a, c, g, t, unknown or other
tttcttggtgaattnttccattttgggta

SEQ ID NO: 1337 - DNA, 29 bp, Homo sapiens
  modified_base (15)..(15): a, c, g, t, unknown or other
acatctagataatgnaactaagaacaaaa

SEQ ID NO: 1338 - DNA, 29 bp, Homo sapiens
  modified_base (15)..(15): a, c, g, t, unknown or other
gataaaaagaggaanggtgatttcacaga

SEQ ID NO: 1339 - DNA, 29 bp, Homo sapiens
  modified_base (15)..(15): a, c, g, t, unknown or other
ttctgagactatttnccaggaatgtagtt

SEQ ID NO: 1340 - DNA, 29 bp, Homo sapiens
  modified_base (15)..(15): a, c, g, t, unknown or other
ttgaattaggtgaanaaatgcatcctccc

SEQ ID NO: 1341 - DNA, 29 bp, Homo sapiens
  modified_base (15)..(15): a, c, g, t, unknown or other
ttacttctgttttgntctctctcagtctt

SEQ ID NO: 1342 - DNA, 29 bp, Homo sapiens
  modified_base (15)..(15): a, c, g, t, unknown or other
ctattactgctttgnaagtactaagaaga

SEQ ID NO: 1343 - DNA, 29 bp, Homo sapiens
  modified_base (15)..(15): a, c, g, t, unknown or other
aaaacccgaaaaggntggactagatgagg

SEQ ID NO: 1344 - DNA, 29 bp, Homo sapiens
  modified_base (15)..(15): a, c, g, t, unknown or other
agttagctatggcantattctgggcaata

SEQ ID NO: 1345 - DNA, 29 bp, Homo sapiens
  modified_base (15)..(15): a, c, g, t, unknown or other
aggtcagtgttcacntggtgtagtcttgc

SEQ ID NO: 1346 - DNA, 29 bp, Homo sapiens
  modified_base (15)..(15): a, c, g, t, unknown or other
acttgccttgcctanagtaacctggtctg

SEQ ID NO: 1347 - DNA, 29 bp, Homo sapiens
  modified_base (15)..(15): a, c, g, t, unknown or other
tctaaagagctaggnatatttttaaaggt

SEQ ID NO: 1348 - DNA, 29 bp, Homo sapiens
  modified_base (15)..(15): a, c, g, t, unknown or other
tacctcagctatccnaaagaactgtgtca

SEQ ID NO: 1349 - DNA, 29 bp, Homo sapiens
  modified_base (15)..(15): a, c, g, t, unknown or other
ctgttttatttaagntgctgcattttggt

SEQ ID NO: 1350 - DNA, 29 bp, Homo sapiens
  modified_base (15)..(15): a, c, g, t, unknown or other
tgtgtctaatcccanttccacggccttcc

SEQ ID NO: 1351 - DNA, 29 bp, Homo sapiens
  modified_base (15)..(15): a, c, g, t, unknown or other
tgcattatacaaatngcatctttctcaaa

SEQ ID NO: 1352 - DNA, 29 bp, Homo sapiens
  modified_base (15)..(15): a, c, g, t, unknown or other
taggtctccttatantttagccaatgttc

SEQ ID NO: 1353 - DNA, 29 bp, Homo sapiens
  modified_base (15)..(15): a, c, g, t, unknown or other
atgtgttaaagaganacttatgattggct

SEQ ID NO: 1354 - DNA, 29 bp, Homo sapiens
  modified_base (15)..(15): a, c, g, t, unknown or other
tagctaagatatttngaccccagttttat

SEQ ID NO: 1355 - DNA, 29 bp, Homo sapiens
  modified_base (15)..(15): a, c, g, t, unknown or other
aataagtttttgatnatttaaaattactc

SEQ ID NO: 1356 - DNA, 29 bp, Homo sapiens
  modified_base (15)..(15): a, c, g, t, unknown or other
actggagaatctctnctcttgacgttgtg

SEQ ID NO: 1357 - DNA, 29 bp, Homo sapiens
  modified_base (15)..(15): a, c, g, t, unknown or other
tatatatgcaccccnaggtatacatgaat

SEQ ID NO: 1358 - DNA, 29 bp, Homo sapiens
  modified_base (15)..(15): a, c, g, t, unknown or other
ttattactaattttnatggaacacatcta

SEQ ID NO: 1359 - DNA, 29 bp, Homo sapiens
  modified_base (15)..(15): a, c, g, t, unknown or other
gcagagaaatgtttntacttatcaatata

SEQ ID NO: 1360 - DNA, 29 bp, Homo sapiens
  modified_base (15)..(15): a, c, g, t, unknown or other
cccagcctagttacncttaccataaaatg

SEQ ID NO: 1361 - DNA, 29 bp, Homo sapiens
  modified_base (15)..(15): a, c, g, t, unknown or other
actttatctaaaatnaaaaggcattggat

SEQ ID NO: 1362 - DNA, 29 bp, Homo sapiens
  modified_base (15)..(15): a, c, g, t, unknown or other
agaggaccaatttgntagcaacaaaaagt

SEQ ID NO: 1363 - DNA, 29 bp, Homo sapiens
  modified_base (15)..(15): a, c, g, t, unknown or other
tcatttattgggtanaaaaatgccgtaag

SEQ ID NO: 1364 - DNA, 29 bp, Homo sapiens
  modified_base (15)..(15): a, c, g, t, unknown or other
cacagctaggagccntggaagagattctt

SEQ ID NO: 1365 - DNA, 29 bp, Homo sapiens
  modified_base (15)..(15): a, c, g, t, unknown or other
tttttacattaaagnttgactctaaagaa

SEQ ID NO: 1366 - DNA, 29 bp, Homo sapiens
  modified_base (15)..(15): a, c, g, t, unknown or other
aagggatctcactgngtgctgaacatgta

SEQ ID NO: 1367 - DNA, 29 bp, Homo sapiens
  modified_base (15)..(15): a, c, g, t, unknown or other
gttcccgagagattnatgtggtactgtgg

SEQ ID NO: 1368 - DNA, 29 bp, Homo sapiens
  modified_base (15)..(15): a, c, g, t, unknown or other
aatatacatgaaacngttggaactgtgac

SEQ ID NO: 1369 - DNA, 29 bp, Homo sapiens
  modified_base (15)..(15): a, c, g, t, unknown or other
gttccattttgggcntctaagccacccac

SEQ ID NO: 1370 - DNA, 29 bp, Homo sapiens
  modified_base (15)..(15): a, c, g, t, unknown or other
attctttgccattangatgaggtcacatt

SEQ ID NO: 1371 - DNA, 29 bp, Homo sapiens
  modified_base (15)..(15): a, c, g, t, unknown or other
cctcacagttgcccnaacagcgctgcctc

SEQ ID NO: 1372 - DNA, 29 bp, Homo sapiens
  modified_base (15)..(15): a, c, g, t, unknown or other
ataaaaacagatccngcttagtcaagtgt

SEQ ID NO: 1373 - DNA, 29 bp, Homo sapiens
  modified_base (15)..(15): a, c, g, t, unknown or other
tttggcaaatataantagttttaataatg

SEQ ID NO: 1374 - DNA, 29 bp, Homo sapiens
  modified_base (15)..(15): a, c, g, t, unknown or other
tatgtaccatgaggngaatcaaagaacca

SEQ ID NO: 1375 - DNA, 29 bp, Homo sapiens
  modified_base (15)..(15): a, c, g, t, unknown or other
atgtggattgtacangctttcctggagac

SEQ ID NO: 1376 - DNA, 29 bp, Homo sapiens
  modified_base (15)..(15): a, c, g, t, unknown or other
tagtcatcacagcanaacgagttgattat

SEQ ID NO: 1377 - DNA, 29 bp, Homo sapiens
  modified_base (15)..(15): a, c, g, t, unknown or other
tgccaagaaaagagnatgccttatgcaaa

SEQ ID NO: 1378 - DNA, 29 bp, Homo sapiens
  modified_base (15)..(15): a, c, g, t, unknown or other
ttatagacgcctccngtgtagaaaatggc

SEQ ID NO: 1379 - DNA, 29 bp, Homo sapiens
  modified_base (15)..(15): a, c, g, t, unknown or other
ggatgtacctgatgnttgaattaacaaat

SEQ ID NO: 1380 - DNA, 29 bp, Homo sapiens
  modified_base (15)..(15): a, c, g, t, unknown or other
tttcttctgggaaantctttttgtttatt

SEQ ID NO: 1381 - DNA, 29 bp, Homo sapiens
  modified_base (15)..(15): a, c, g, t, unknown or other
tgaggtaatgctagncaagactttatcca

SEQ ID NO: 1382 - DNA, 29 bp, Homo sapiens
  modified_base (15)..(15): a, c, g, t, unknown or other
cttacccaaaagacngagttactactctc

SEQ ID NO: 1383 - DNA, 29 bp, Homo sapiens
  modified_base (15)..(15): a, c, g, t, unknown or other
aaagccaaagttccntttcatgggctgca

SEQ ID NO: 1384 - DNA, 29 bp, Homo sapiens
  modified_base (15)..(15): a, c, g, t, unknown or other
tagtttagttcctanagaaggatagtaaa

SEQ ID NO: 1385 - DNA, 29 bp, Homo sapiens
  modified_base (15)..(15): a, c, g, t, unknown or other
agatgtatctatcantctgcataaatacg

SEQ ID NO: 1386 - DNA, 29 bp, Homo sapiens
  modified_base (15)..(15): a, c, g, t, unknown or other
tcatgttacatattntagcctcataaata

SEQ ID NO: 1387 - DNA, 29 bp, Homo sapiens
  modified_base (15)..(15): a, c, g, t, unknown or other
acaataaaataaggntcatacagcttggg

SEQ ID NO: 1388 - DNA, 29 bp, Homo sapiens
  modified_base (15)..(15): a, c, g, t, unknown or other
tcacgaaaatcatangactctacggaaag

SEQ ID NO: 1389 - DNA, 29 bp, Homo sapiens
  modified_base (15)..(15): a, c, g, t, unknown or other
cacattaattaagangtgaaggttggcaa

SEQ ID NO: 1390 - DNA, 29 bp, Homo sapiens
  modified_base (15)..(15): a, c, g, t, unknown or other
tgcatgcttgcatcnatactcaaatatta

SEQ ID NO: 1391 - DNA, 29 bp, Homo sapiens
  modified_base (15)..(15): a, c, g, t, unknown or other
ttcatcctgactcanttctctaagtatat

SEQ ID NO: 1392 - DNA, 29 bp, Homo sapiens
  modified_base (15)..(15): a, c, g, t, unknown or other
ccaattaagtttacntctcaatattttaa

SEQ ID NO: 1393 - DNA, 29 bp, Homo sapiens
  modified_base (15)..(15): a, c, g, t, unknown or other
ctctcaagtgtaagntttcaaggcactcg

SEQ ID NO: 1394 - DNA, 29 bp, Homo sapiens
  modified_base (15)..(15): a, c, g, t, unknown or other
ggatggcctccatgnaagagtaaaatgaa

SEQ ID NO: 1395 - DNA, 29 bp, Homo sapiens
  modified_base (15)..(15): a, c, g, t, unknown or other
aaaggaaaaaatccnttacagctattaaa

SEQ ID NO: 1396 - DNA, 29 bp, Homo sapiens
  modified_base (15)..(15): a, c, g, t, unknown or other
tctgctctgtaaganatcaacaaacattt

SEQ ID NO: 1397 - DNA, 29 bp, Homo sapiens
  modified_base (15)..(15): a, c, g, t, unknown or other
ttactgagaaagcangcaagcattcaata

SEQ ID NO: 1398 - DNA, 29 bp, Homo sapiens
  modified_base (15)..(15): a, c, g, t, unknown or other
atgtgtctgaaattntattatttgtctat

SEQ ID NO: 1399 - DNA, 29 bp, Homo sapiens
  modified_base (15)..(15): a, c, g, t, unknown or other
aatctgggactaccnagggtgctgaagac

SEQ ID NO: 1400 - DNA, 29 bp, Homo sapiens
  modified_base (15)..(15): a, c, g, t, unknown or other
ttaactgattataanttttctgtcagttt

SEQ ID NO: 1401 - DNA, 29 bp, Homo sapiens
  modified_base (15)..(15): a, c, g, t, unknown or other
tccaataatttcagnaagccgtcatatta

SEQ ID NO: 1402 - DNA, 29 bp, Homo sapiens
  modified_base (15)..(15): a, c, g, t, unknown or other
gctaacttcctgganatatgaactatatt

SEQ ID NO: 1403 - DNA, 29 bp, Homo sapiens
  modified_base (15)..(15): a, c, g, t, unknown or other
tttgagattcatgangctttgaggattct

SEQ ID NO: 1404 - DNA, 29 bp, Homo sapiens
  modified_base (15)..(15): a, c, g, t, unknown or other
atctgttggaggcantaacttcttacata

SEQ ID NO: 1405 - DNA, 29 bp, Homo sapiens
  modified_base (15)..(15): a, c, g, t, unknown or other
gattagtggtctacnatagcaattctttt

SEQ ID NO: 1406 - DNA, 29 bp, Homo sapiens
  modified_base (15)..(15): a, c, g, t, unknown or other
ctttgagggcaactnaacactctggctca

SEQ ID NO: 1407 - DNA, 29 bp, Homo sapiens
  modified_base (15)..(15): a, c, g, t, unknown or other
aagaatttcttaaanagtatgccaaactt

SEQ ID NO: 1408 - DNA, 29 bp, Homo sapiens
  modified_base (15)..(15): a, c, g, t, unknown or other
taatgttgtcatcanacacagttcatagg

SEQ ID NO: 1409 - DNA, 29 bp, Homo sapiens
  modified_base (15)..(15): a, c, g, t, unknown or other
atattgtactggccntatcaaaacagaaa

SEQ ID NO: 1410 - DNA, 29 bp, Homo sapiens
  modified_base (15)..(15): a, c, g, t, unknown or other
tggcatggactgggngagctctaaggact

SEQ ID NO: 1411 - DNA, 29 bp, Homo sapiens
  modified_base (15)..(15): a, c, g, t, unknown or other
gtgttcatcttctcnagggaaacctggcc

SEQ ID NO: 1412 - DNA, 29 bp, Homo sapiens
  modified_base (15)..(15): a, c, g, t, unknown or other
ctttaaaattggttntgatgctgacctac

SEQ ID NO: 1413 - DNA, 29 bp, Homo sapiens
  modified_base (15)..(15): a, c, g, t, unknown or other
tgactcagccctggnatgtttacagccta

SEQ ID NO: 1414 - DNA, 29 bp, Homo sapiens
  modified_base (15)..(15): a, c, g, t, unknown or other
cttttgcaccctttnggtaccagaagctc

SEQ ID NO: 1415 - DNA, 29 bp, Homo sapiens
  modified_base (15)..(15): a, c, g, t, unknown or other
gaagtcactgtgatnaggggagatgtctg

SEQ ID NO: 1416 - DNA, 29 bp, Homo sapiens
  modified_base (15)..(15): a, c, g, t, unknown or other
ctctcctctcacacnggcctactgctgcc

SEQ ID NO: 1417 - DNA, 29 bp, Homo sapiens
  modified_base (15)..(15): a, c, g, t, unknown or other
tgtgttaatgtatangtagactttaaact

SEQ ID NO: 1418 - DNA, 29 bp, Homo sapiens
  modified_base (15)..(15): a, c, g, t, unknown or other
aatttcccaggcagntgaaaaggttccag

SEQ ID NO: 1419 - DNA, 29 bp, Homo sapiens
  modified_base (15)..(15): a, c, g, t, unknown or other
tgcagttcagctttnaaaaaatgttccac

SEQ ID NO: 1420 - DNA, 29 bp, Homo sapiens
  modified_base (15)..(15): a, c, g, t, unknown or other
gacgatataatgggnacagtgcctggcaa

SEQ ID NO: 1421 - DNA, 29 bp, Homo sapiens
  modified_base (15)..(15): a, c, g, t, unknown or other
ctccttaagacttanaattgtttggccaa

SEQ ID NO: 1422 - DNA, 29 bp, Homo sapiens
  modified_base (15)..(15): a, c, g, t, unknown or other
tgacctaacttcctnttttagcggctgcc

SEQ ID NO: 1423 - DNA, 29 bp, Homo sapiens
  modified_base (15)..(15): a, c, g, t, unknown or other
tgtaaggcctggagntcttgtgaggctgt

SEQ ID NO: 1424 - DNA, 29 bp, Homo sapiens
  modified_base (15)..(15): a, c, g, t, unknown or other
aggatatcggtgatnaaagtatttgaaaa

SEQ ID NO: 1425 - DNA, 29 bp, Homo sapiens
  modified_base (15)..(15): a, c, g, t, unknown or other
tttagaagaggactncaaattaaccttat

SEQ ID NO: 1426 - DNA, 29 bp, Homo sapiens
  modified_base (15)..(15): a, c, g, t, unknown or other
cagtcctcagctcangtctacttggttct

SEQ ID NO: 1427 - DNA, 29 bp, Homo sapiens
  modified_base (15)..(15): a, c, g, t, unknown or other
tcagtaactaccgtnctacaaaaactcaa

SEQ ID NO: 1428 - DNA, 29 bp, Homo sapiens
  modified_base (15)..(15): a, c, g, t, unknown or other
gttaactgggtgaangtcttgcctttata

SEQ ID NO: 1429 - DNA, 29 bp, Homo sapiens
  modified_base (15)..(15): a, c, g, t, unknown or other
tgccataaggaacangctgttagccagaa

SEQ ID NO: 1430 - DNA, 29 bp, Homo sapiens
  modified_base (15)..(15): a, c, g, t, unknown or other
aagtatgaaaaacanttctctcttcatga

SEQ ID NO: 1431 - DNA, 29 bp, Homo sapiens
  modified_base (15)..(15): a, c, g, t, unknown or other
ttcttattctttttnttgataactgtaaa

SEQ ID NO: 1432 - DNA, 29 bp, Homo sapiens
  modified_base (15)..(15): a, c, g, t, unknown or other
gagagatcccttcantctggaaagctgac

SEQ ID NO: 1433 - DNA, 29 bp, Homo sapiens
  modified_base (15)..(15): a, c, g, t, unknown or other
cttctgttactcatntaagctgctgtcct

SEQ ID NO: 1434 - DNA, 29 bp, Homo sapiens
  modified_base (15)..(15): a, c, g, t, unknown or other
aaggctaaccttggnttccccagctaagc

SEQ ID NO: 1435 - DNA, 29 bp, Homo sapiens
  modified_base (15)..(15): a, c, g, t, unknown or other
agagttggaaagcanccatcattattatc

SEQ ID NO: 1436 - DNA, 29 bp, Homo sapiens
  modified_base (15)..(15): a, c, g, t, unknown or other
gtaaatggcaagacngaatcagttccgga

SEQ ID NO: 1437 - DNA, 29 bp, Homo sapiens
  modified_base (15)..(15): a, c, g, t, unknown or other
gcagaaaagcagtgnaccatgtggttttg

SEQ ID NO: 1438 - DNA, 29 bp, Homo sapiens
  modified_base (15)..(15): a, c, g, t, unknown or other
gatgggggatagccntgattgttgagcaa

SEQ ID NO: 1439 - DNA, 29 bp, Homo sapiens
  modified_base (15)..(15): a, c, g, t, unknown or other
gtgaagcgtaggacnggatacccagagct

SEQ ID NO: 1440 - DNA, 29 bp, Homo sapiens
  modified_base (15)..(15): a, c, g, t, unknown or other
tgagtttaacttccncgccttgctttcct

SEQ ID NO: 1441 - DNA, 29 bp, Homo sapiens
  modified_base (15)..(15): a, c, g, t, unknown or other
ggttctttccaatanctccttttggagct

SEQ ID NO: 1442 - DNA, 29 bp, Homo sapiens
  modified_base (15)..(15): a, c, g, t, unknown or other
cagaagctctctccnccttaatctcaggt

SEQ ID NO: 1443 - DNA, 29 bp, Homo sapiens
  modified_base (15)..(15): a, c, g, t, unknown or other
tgctgtgaacatccntgtgctcctgggct

SEQ ID NO: 1444 - DNA, 29 bp, Homo sapiens
  modified_base (15)..(15): a, c, g, t, unknown or other
gtgacaggttagganacagatgaggccct

SEQ ID NO: 1445 - DNA, 29 bp, Homo sapiens
  modified_base (15)..(15): a, c, g, t, unknown or other
tagagttgcaggcanttttgaaagaagga

SEQ ID NO: 1446 - DNA, 29 bp, Homo sapiens
  modified_base (15)..(15): a, c, g, t, unknown or other
cataaattgtctganaggatctccaccat

SEQ ID NO: 1447 - DNA, 29 bp, Homo sapiens
  modified_base (15)..(15): a, c, g, t, unknown or other
tttcactgaaaaccntcactctaccctct

SEQ ID NO: 1448 - DNA, 29 bp, Homo sapiens
  modified_base (15)..(15): a, c, g, t, unknown or other
gcgatctcccgttcnaatcaaaatcaaac

SEQ ID NO: 1449 - DNA, 29 bp, Homo sapiens
  modified_base (15)..(15): a, c, g, t, unknown or other
gatatgcagtgctgngtaatggactgtgt

SEQ ID NO: 1450 - DNA, 29 bp, Homo sapiens
  modified_base (15)..(15): a, c, g, t, unknown or other
ctgggagccaaagtnctcaatggagaaca

SEQ ID NO: 1451 - DNA, 29 bp, Homo sapiens
  modified_base (15)..(15): a, c, g, t, unknown or other
taatagcccagttgntatggctaaattaa

SEQ ID NO: 1452 - DNA, 29 bp, Homo sapiens
  modified_base (15)..(15): a, c, g, t, unknown or other
ctaaattatgttgtntactagcccagaga

SEQ ID NO: 1453 - DNA, 29 bp, Homo sapiens
  modified_base (15)..(15): a, c, g, t, unknown or other
cactgtcatctctcntatgaatcactctt

SEQ ID NO: 1454 - DNA, 29 bp, Homo sapiens
  modified_base (15)..(15): a, c, g, t, unknown or other
agtaatttgtcgcanttttataccactgg

SEQ ID NO: 1455 - DNA, 29 bp, Homo sapiens
  modified_base (15)..(15): a, c, g, t, unknown or other
cgtttgtgggttagncctccatggattta

SEQ ID NO: 1456 - DNA, 29 bp, Homo sapiens
  modified_base (15)..(15): a, c, g, t, unknown or other
aattcagttgcacancagtgtcagtggaa

SEQ ID NO: 1457 - DNA, 29 bp, Homo sapiens
  modified_base (15)..(15): a, c, g, t, unknown or other
aatatatcttaattnaaaagggctgcttg

SEQ ID NO: 1458 - DNA, 29 bp, Homo sapiens
  modified_base (15)..(15): a, c, g, t, unknown or other
caggaaggacaggcncctagtaaggatgt

SEQ ID NO: 1459 - DNA, 29 bp, Homo sapiens
  modified_base (15)..(15): a, c, g, t, unknown or other
accgagctggacttntccgtggtctggga

SEQ ID NO: 1460 - DNA, 29 bp, Homo sapiens
  modified_base (15)..(15): a, c, g, t, unknown or other
gggggccctagaacnctcctcctcctgag

(19) United States
(12) Patent Application Publication
(10) Pub. No.: US 2008/0131893 A1
(43) Pub. Date: Jun. 5, 2008

(54) Predicting Parkinson's Disease

(75) Inventors: Demetrius M. Maraganore, Rochester, MN (US); Timothy G. Lesnick, Rochester, MN (US)

Correspondence Address: FISH & RICHARDSON P.C., PO BOX 1022, MINNEAPOLIS, MN, 55440-1022, US

(73) Assignee: MAYO FOUNDATION FOR MEDICAL EDUCATION AND RESEARCH, Rochester, MN (US)

(21) Appl. No.: 11/849,090

(22) Filed: Aug. 31, 2007

Related U.S. Application Data
(60) Provisional application No. 60/842,054, filed on Aug. 31, 2006.

(51) Int. Cl. C12Q 1/68

(52) U.S. Cl. 435/6

(57) ABSTRACT

This document relates to methods and materials involved in predicting Parkinson's disease. For example, methods for assessing the genotype of a human to determine whether or not the human has an increased susceptibility of developing Parkinson's disease are provided. In addition, diagnostic devices containing probe or primer collections designed to detect the genotype of a human, thereby providing the ability to assess the human for increased susceptibility to develop Parkinson's disease, also are provided.

### CROSS-REFERENCE TO RELATED APPLICATION

This application claims the benefit of U.S. Provisional Application Ser. No. 60/842,054, filed Aug. 31, 2006; which is incorporated by reference in its entirety.

### STATEMENT AS TO FEDERALLY SPONSORED RESEARCH

Funding for the work described herein was provided by the federal government under grant numbers ES10751 and NS33978 awarded by the National Institute of Health. The federal government has certain rights in the invention.

### BACKGROUND

1. Technical Field

This document relates to methods and materials involved in predicting Parkinson's disease.

2. Background Information

Complex diseases occur commonly in the population and are a major source of disability and death in societies worldwide. They are thought to arise from multiple predisposing factors, both genetic and non-genetic, and joint effects of those factors are thought to be of key importance. Parkinson's disease (PD) serves as an example of a complex disease. Other examples include Alzheimer's disease, diabetes mellitus, nicotine and alcohol dependence, and many cancers. While major inroads have been made in identifying the genetic causes of rare Mendelian disorders, little progress has been made in the discovery of gene variations that predispose to complex diseases. The single gene variants that have been shown to associate reproducibly with complex diseases typically have small effect sizes.

### SUMMARY

This document relates to methods and materials involved in predicting PD. For example, this document provides methods for assessing the genotype of a human to determine whether or not the human has an increased susceptibility of developing PD. In addition, this document provides diagnostic devices containing probe or primer collections designed to detect the genotype of a human, thereby providing the ability to assess the human for increased susceptibility to develop PD.

As described herein, common variations (e.g., polymorphisms) within genes that encode polypeptides within an axon guidance pathway can be strong predictors of phenotypes such as PD susceptibility and age at onset of PD. For example, the models provided herein can be used to predict PD susceptibility (P=4.64×10⁻³⁸), survival free of PD (P=5.43×10⁻⁴⁸), and age at PD onset (P=1.68×10⁻⁵¹). As demonstrated herein, polymorphisms in axon guidance pathway genes accounted for nearly 70% of the age at onset variance of PD, and people with high versus low model scores differed in survival free of PD by more than 20 years. The results provided herein help demonstrate that complex genetics can be a paradigm for common, late onset diseases in man.

In general, one aspect of this document features a method for assessing Parkinson's disease susceptibility. The method comprises, or consists essentially of, determining whether or not a human contains an axon guidance pathway genotype predisposing the human to develop Parkinson's disease. The human can be any age (e.g., 40 years of age or older). The determining step can comprise sequencing nucleic acid from the human. The determining step can comprise using a probe to determine the presence or absence of a polymorphism in the human. The genotype can comprise at least two of the polymorphisms of Table 1.

In another aspect, this document features a method for predicting age of Parkinson's disease onset in a human. The method comprises, or consists essentially of:

(a) determining the presence or absence of polymorphisms in a set of axon guidance pathway genes of the human to obtain information about the axon guidance pathway genotype of the human, and

(b) calculating an age of Parkinson's disease onset for the human based on the information. The human can be any age (e.g., 20 years of age or older). The determining step can comprise sequencing nucleic acid from the human. The determining step can comprise using a probe to determine the presence or absence of a polymorphism in the human. The genotype can comprise at least two of the polymorphisms of Table 3.

In another aspect, this document features a method for assessing a human for the likelihood of survival free of Parkinson's disease. The method comprises, or consists essentially of, determining whether or not the human contains an axon guidance pathway genotype indicative of survival free of Parkinson's disease. The human can be any age (e.g., 40 years of age or older). The determining step can comprise sequencing nucleic acid from the human. The determining step can comprise using a probe to determine the presence or absence of a polymorphism in the human. The genotype can comprise at least two of the polymorphisms of Table 2.

In another aspect, this document features a diagnostic device for assessing a human's predisposition to develop Parkinson's disease. The device comprises, or consists essentially of, an array of nucleic acid probes, wherein each nucleic acid probe of the array is located at an identifiable location of the device, wherein at least 50 percent of the probes of the device are selected from the group of probes consisting of probes having a sequence capable of hybridizing to a polymorphic nucleic acid set forth in Table 1, 2, or 3. The array can comprise at least 25 nucleic acid probes. The array can comprise at least 50 nucleic acid probes. The array can comprise at least 100 nucleic acid probes. At least 75 percent of the probes of the device are selected from the group. At least 90 percent of the probes of the device are selected from the group. At least 50 percent of the probes of the device are selected from the group of probes consisting of probes having a sequence capable of hybridizing to a polymorphic nucleic acid set forth in Table 1. At least 50 percent of the probes of the device are selected from the group of probes consisting of probes having a sequence capable of hybridizing to a polymorphic nucleic acid set forth in Table 2. At least 50 percent of the probes of the device are selected from the group of probes consisting of probes having a sequence capable of hybridizing to a polymorphic nucleic acid set forth in Table 3.

In another aspect, this document features a method for assessing Parkinson's disease susceptibility. The method comprises, or consists essentially of, determining whether or not a human contains a polymorphism in PPP3CA, MRAS, PLXNA2, RAC2, SEMA5A, NFATC2, ROBO1, ROBO2, EPHB1, NFATC4, NTNG1, or EFNA5 nucleic acid (or a polymorphism in any of the brain expressed axon guidance pathway genes of Table 20 or as annotated in KEGG (World Wide Web at “genome.jp/kegg/pathway/hsa/hsa04360.html”)), wherein the presence of the polymorphism indicates that the human is susceptible to developing Parkinson's disease. The method can comprise diagnosing the human as being susceptible to develop Parkinson's disease if the human contains the polymorphism, and diagnosing the human as not being susceptible to develop Parkinson's disease if the human lacks the polymorphism. The method can comprise recording that the human as being susceptible to develop Parkinson's disease if the human contains the polymorphism, and recording that the human as not being susceptible to develop Parkinson's disease if the human lacks the polymorphism.

In another aspect, this document features a method for assessing Parkinson's disease susceptibility. The method comprises using a model to determine whether or not a human contains an axon guidance pathway genotype predisposing the human to develop Parkinson's disease, wherein the model comprises a model concordance of at least 0.6 (e.g., greater than 0.65, greater than 0.7, greater than 0.75, greater than 0.8, between 0.6 and 0.99, between 0.65 and 0.99, or between 0.65 and 0.95), an r-squared of at least 0.6 (e.g., greater than 0.65, greater than 0.7, greater than 0.75, greater than 0.8, between 0.6 and 0.99, between 0.65 and 0.99, or between 0.65 and 0.95), or a sensitivity and specificity of at least 0.6 (e.g., greater than 0.65, greater than 0.7, greater than 0.75, greater than 0.8, between 0.6 and 0.99, between 0.65 and 0.99, or between 0.65 and 0.95).

In another aspect, this document features a method for diagnosing a brain disorder or assessing brain disorder susceptibility. The method comprises using a model to determine whether or not a human contains an axon guidance pathway genotype diagnositic for the brain disorder or predisposing the human to develop the brain disorder, wherein the model comprises a model concordance of at least 0.6 (e.g., greater than 0.65, greater than 0.7, greater than 0.75, greater than 0.8, between 0.6 and 0.99, between 0.65 and 0.99, or between 0.65 and 0.95), an r-squared of at least 0.6 (e.g., greater than 0.65, greater than 0.7, greater than 0.75, greater than 0.8, between 0.6 and 0.99, between 0.65 and 0.99, or between 0.65 and 0.95), or a sensitivity and specificity of at least 0.6 (e.g., greater than 0.65, greater than 0.7, greater than 0.75, greater than 0.8, between 0.6 and 0.99, between 0.65 and 0.99, or between 0.65 and 0.95). The brain disorder can be selected from the group consisting of Parkinson's disease, Alzheimer's disease, ALS, Tourette's syndrome, dyslexia, autism, mental retardation, epilepsy, stuttering, schizophrenia, addiction, anxiety, depression, obsessive-compulsive disorder, ADHD, MS, brain tumors, brain injury, and spinal cord injury.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 contains two graphs of goodness-of-fit model results using axon guidance genes to predict susceptibility to PD. (Top) Histogram of predicted probabilities of PD in cases. (Bottom) Histogram of predicted probabilities of PD in unaffected siblings. A perfect fit would have all predicted probabilities for cases equal to one, and all predicted probabilities for unaffected siblings equal to zero. A model with no explanatory value would have histograms which were indistinguishable from each other.

FIG. 2 is a Kaplan-Meier survival plot for age at onset in PD patients, grouped by categorized risk score from proportional hazards model. The model clearly differentiates between early age at onset cases and late age at onset cases. Cases generating the long-dashed line were predicted to be at highest risk for early onset of PD, followed by the medium-dashed, short-dashed, and continuous lines, respectively. Since only cases were included, all of the lines end at zero percent free of PD.

FIG. 3 is a graph plotting predicted vs. reported age at onset in PD patients. Points indicate reported individual values, and the line represents perfect agreement. In this case, the pattern shows a fairly tight elliptical pattern, reflecting a good fit and high R² of 0.68. The fit appears equally good from the minimum to maximum reported age at onset.

FIG. 4 contains grafts plotting the distributions of the test statistics from the models with randomly selected SNPs and the values of the test statistics from final models for PD susceptibility (A), survival free of PD (B), and age at onset of PD (C), within the primary whole-genome association dataset.

FIG. 5 contains a table of convergence of findings for whole-genome association and gene expression profiling datasets. Listed are genes that had one or more SNPs included in at least one of the three genetic models that predicted PD susceptibility or survival free of PD or age at onset of PD (n=45 genes). Expression data are summarized for each of the three nigrostriatal regions (substantia nigra, putamen, and caudate). The number 1 indicates that the gene had no difference in expression in cases and controls. The number 2 indicates that there were no informative probe sets for the gene and region. The number 3 indicates that the gene had increased expression in cases compared to controls. The number 4 indicates that the gene had reduced expression in cases compared to controls.

FIG. 6 is a flowchart of a procedure used to build joint action models using SNPs from genes in the axon guidance pathway that predict PD susceptibility, survival free of PD, and age at onset of PD, within both whole-genome association datasets.

FIG. 7 contains two graphs of goodness-of-fit final model results using axon guidance genes to predict susceptibility to PD, within a second whole-genome association dataset. Histogram of predicted probabilities of PD in cases (top). Histogram of predicted probabilities of PD in unrelated controls (bottom). A perfect fit would have all predicted probabilities for cases equal to one, and all predicted probabilities for unrelated controls equal to zero. A model with no explanatory value would have histograms which were indistinguishable from each other.

FIG. 8 is a Kaplan-Meier survival plot for age at onset in PD patients, grouped by categorized risk score from proportional hazards model, within a second whole-genome association dataset. The model clearly differentiates between early age at onset cases and late age at onset cases. Cases generating the long-dashed line were predicted to be at highest risk for early onset of PD, followed by the medium-dashed, short-dashed, and continuous lines respectively. Since only cases were included, all of the lines end at zero percent free of PD.

FIG. 9 is graph plotting predicted vs. reported age at onset in PD patients, within a second whole-genome association dataset. Points indicate reported individual values, and the line represents perfect agreement. In this case, the pattern reveals a fairly tight elliptical pattern, reflecting a good fit and high R² of 0.71. The fit appears equally good from the minimum to maximum reported age at onset. By contrast to the primary dataset (which reported age at onset to the month), the second dataset reported age at onset to the year. This and smaller sample size may account for the slightly grainy appearance of the plot for this secondary study as compared to the primary study.

FIG. 10. Goodness-of-Fit of Final Model Using Axon Guidance Genes to Predict Susceptibility to ALS. Histogram of predicted probabilities of ALS in cases (A). Histogram of predicted probabilities of ALS in controls (B). A perfect fit would have all predicted probabilities for cases equal to one, and all predicted probabilities for controls equal to zero. A model with no explanatory value would have histograms which were indistinguishable from each other.

FIG. 11. Kaplan-Meier Survival Plot for Age at Onset in ALS Patients, Grouped by Categorized Risk Score from Proportional Hazards Model. The model clearly differentiates between early age at onset cases and late age at onset cases. Cases generating the long-dashed line were predicted to be at highest risk for early onset of ALS, followed by the medium-dashed, short-dashed, and continuous lines respectively. Since only cases were included, all of the lines end at zero percent free of ALS.

FIG. 12. Predicted vs. Reported Age at Onset in ALS Patients. Points indicate reported individual values, and the line represents perfect agreement. In this case, the pattern shows a fairly tight elliptical pattern, reflecting a good fit and high R² of 0.86. The fit appears equally good from the minimum to maximum reported age at onset.

FIG. 13 is a flowchart of a procedure used to construct joint action models using SNPs from genes in the axon guidance pathway that predict ALS susceptibility, survival free of ALS, and age at onset of ALS.

FIG. 14. Goodness-of-Fit of Final Model Using Axon Guidance Genes to Predict Susceptibility to PD. Histogram of predicted probabilities of PD in cases (A). Histogram of predicted probabilities of PD in controls (B). A perfect fit would have all predicted probabilities for cases equal to one, and all predicted probabilities for controls equal to zero. A model with no explanatory value would have histograms which were indistinguishable from each other.

FIG. 15. Kaplan-Meier Survival Plot for Age at Onset in PD Patients, Grouped by Categorized Risk Score from Proportional Hazards Model. The model clearly differentiates between early age at onset cases and late age at onset cases. Cases generating the long-dashed line were predicted to be at highest risk for early onset of PD, followed by the medium-dashed, short-dashed, and continuous lines respectively. Since only cases were included, all of the lines end at zero percent free of PD.

FIG. 16. Predicted vs. Reported Age at Onset in PD Patients. Points indicate reported individual values, and the line represents perfect agreement. In this case, the pattern shows a fairly tight elliptical pattern, reflecting a good fit and high R² of 0.86. The fit appears equally good from the minimum to maximum reported age at onset.

FIG. 17. Distributions of the Significant SNPs in the Genes from the ALS and PD Final Models. This figure shows results from final models for susceptibility (a), survival free of disease (b), and age at onset of disease (c). The bars on the right represent ALS, and the bars bars of the left represent PD.

### DETAILED DESCRIPTION

This document provides methods and materials related to assessing PD. For example, this document provides methods for assessing the genotype of a human to determine whether or not the human has an increased susceptibility of developing PD. In addition, this document provides diagnostic devices containing probe or primer collections designed to detect the genotype of a human, thereby providing the ability to assess the human for increased susceptibility to develop PD.

In addition to PD, the methods and materials provided herein can be used to assess other brain disorders including, without limitation, Alzheimer's disease, ALS, Tourette's syndrome, dyslexia, autism, mental retardation, epilepsy, stuttering, schizophrenia, addiction, anxiety, depression, obsessive-compulsive disorder, ADHD, MS, brain tumors, brain injury, and spinal cord injury. For example, the methods and materials provided herein can be used to diagnose a brain disorder (e.g., PD) or to assess brain disorder susceptibility in a mammal.

Any type of sample containing a human's nucleic acid can be used to determine the human's genotype. For example, cells such as white blood cells or skin cells can be collected and assessed for the presence or absence of polymorphisms such as those described in Table 1, 2, 3, 5, 6, 7, 13, 14, 15, 17, 18, or 19. In some cases, a polymorphism located near (e.g., a polymorphism considered linked to another polymorphism) one or more of the polymorphisms set forth in Table 1, 2, 3, 5, 6, 7, 13, 14, 15, 17, 18, or 19 can be used to determine the human's genotype. In some cases, a biopsy (e.g., punch biopsy, aspiration biopsy, excision biopsy, needle biopsy, or shave biopsy), tissue section, lymph fluid sample, blood sample, or synovial fluid sample can be used.

Any method can be used to determine whether or not a particular polymorphism or collection of polymorphisms are present within a human's genome. For example, sequencing techniques or hybridization techniques (e.g., chip hybridization techniques) can be performed to detect polymorphisms set forth in Table 1, 2, 3, 5, 6, 7, 13, 14, 15, 17, 18, or 19. Methods for chip hybridization assays include, without limitation, methods that involve using nucleic acid arrays having probes designed to distinguish between different polymorphic sequences via hybridization. Such methods can be used to determine simultaneously the presence or absence of multiple polymorphisms.

The methods and materials provided herein can be used at any time during a human's life to determine whether or not the human is susceptible to developing a brain disorder (e.g., PD or ALS). In some cases, the methods and materials provided herein can be used to assess a human embryo, fetus, newborn, infant, child, or adult. For example, a sample can be obtained from a human at least 20, 30, 40, 50, 60, or more years of age. Once obtained, the sample can be evaluated to determine the human's genotype (e.g., the human's genotype regarding the SNPs listed in Table 1, 2, 3, 5, 6, 7, 13, 14, 15, 17, 18, or 19).

This description also provides nucleic acid arrays. The arrays provided herein can be two-dimensional arrays, and can contain at least 10 different nucleic acid molecules (e.g., at least 20, at least 30, at least 50, at least 100, or at least 200 different nucleic acid molecules). Each nucleic acid molecule can have any length. For example, each nucleic acid molecule can be between 10 and 250 nucleotides (e.g., between 12 and 200, 14 and 175, 15 and 150, 16 and 125, 18 and 100, 20 and 75, or 25 and 50 nucleotides) in length. In addition, each nucleic acid molecule can have any sequence. For example, the nucleic acid molecules of the arrays provided herein can contain sequences capable of detecting the polymorphisms set forth in Table 1, 2, 3, 5, 6, 7, 13, 14, 15, 17, 18, or 19. Typically, at least 25% (e.g., at least 30%, at least 40%, at least 50%, at least 60%, at least 75%, at least 80%, at least 90%, at least 95%, or 100%) of the nucleic acid molecules of an array provided herein can contain a sequence capable of detecting a polymorphism set forth in Table 1, 2, 3, 5, 6, 7, 13, 14, 15, 17, 18, or 19.

The nucleic acid arrays provided herein can contain nucleic acid molecules attached to any suitable surface (e.g., plastic or glass). In addition, any method can be use to make a nucleic acid array. For example, spotting techniques and in situ synthesis techniques can be used to make nucleic acid arrays. Further, the methods disclosed in U.S. Pat. Nos. 5,744,305 and 5,143,854 can be used to make nucleic acid arrays.

TABLE 20
Axon guidance genes.

| | Kegg | |
|---|---|---|
| | Axon | |
| Kegg Axon Guidance | Guidance | |
| Pathway Gene | listing | Chromosome |
| SRGAP3/SRGAP2 | srGAP | 3 |
| LRRC4C | NGL-1 | 11 |
| L1CAM/MASA | L1CAM | 23 |
| SEMA3G | Sema3A | 3 |
| KRAS/KRAS2 | Ras | 12 |
| SRGAP2/FNBP2/srGAP3 | srGAP | 1 |
| ABL1/ABL | Abl | 9 |
| ABLIM1 | Ablim | 10 |
| ABLIM2 | Ablim | 4 |
| ABLIM3 | Ablim | 5 |
| ARHGEF12 | LARG | 11 |
| CDC42 | Cdc42 | 1 |
| CDK5 | CDK5 | 7 |
| CFL2 | Cofilin | 14 |
| CHP | CALN | 15 |
| CXCL12/SDF1A/SDF1B/SDF | CXCL12 | 10 |
| CXCR4 | CXCR4 | 2 |
| DCC | DCC | 18 |
| DPYSL2 | CRMP | 8 |
| DPYSL5 | CRAM | 2 |
| EFNA1/TNFAIP4/EPLG1 | Ephrin-A | 1 |
| EFNA5 | Ephrin-A | 5 |
| EFNB1 | Ephrin-B | 23 |
| EFNB2 | Ephrin-B | 13 |
| EPHA2/ECK | EphA | 1 |
| EPHA3/ETK/ETK1/TYRO4 | EphA | 3 |
| EPHA4 | EphA | 2 |
| EPHA5 | EphA | 4 |
| EPHA7 | EphA | 6 |
| EPHA8/EEK | EphA | 1 |
| EPHB1/EPHT2 | EphB | 3 |
| EPHB2/DRT/ERK/EPHT3 | EphB | 1 |
| EPHB4/HTK | EphB | 7 |
| EPHB6 | EphB | 7 |
| FYN | Fyn | 6 |
| GNAI1 | Gi | 7 |
| GNAI2 | Gi | 3 |
| GNAI3 | Gi | 1 |
| GSK3B | GSK3β | 3 |
| HRAS/HRAS1 | Ras | 11 |
| ITGB1/FNRB/MSK12/MDF2 | ITGB1 | 10 |
| LIMK1 | LIMK | 7 |
| LIMK2 | LIMK | 22 |
| MAPK1 | ERK | 22 |
| MET | MET | 7 |
| MRAS | Ras | 3 |
| NCK1 | Nck1 | 3 |
| NCK2 | Grb4 | 2 |
| NFAT5 | NFAT | 16 |
| NFATC1 | NFAT | 18 |
| NFATC2 | NFAT | 20 |
| NFATC3 | NFAT | 16 |
| NFATC4 | NFAT | 14 |
| NGEF | Ephexin | 2 |
| NRAS | Ras | 1 |
| NRP1 | NRP1 | 10 |
| NTN1 | Netrin1 | 17 |
| NTN4 | Netrin4 | 12 |
| NTNG1 | NetrinG | 1 |
| PAK1 | PAK | 11 |
| PAK2 | PAK | 3 |
| PAK3 | PAK | 23 |
| PAK4 | PAK | 19 |
| PAK6 | PAK | 15 |
| PAK7 | PAK | 20 |
| PLXNA1/PLXN1 | Plexin A | 3 |
| PLXNA2 | Plexin A | 1 |
| PLXNA3/PLXN4 | Plexin A | 23 |
| PLXNB1 | Plexin B | 3 |
| PLXNB3/PLXN6 | Plexin B | 23 |
| PLXNC1 | Plexin C | 12 |
| PPP3CA | CALN | 4 |
| PPP3CB/CALNB | CALN | 10 |
| PPP3CC | CALN | 8 |
| PPP3R1 | CALN | 2 |
| PPP3R2 | CALN | 9 |
| PTK2 | FAK | 8 |
| RAC1 | Rac | 7 |
| RAC2 | Rac | 22 |
| RASA1 | RasGAP/ | 5 |
| | p120 | |
| RGS3 | RGS3 | 9 |
| RHOA/ARH12/ARHA | Rhoa | 3 |
| RHOD | RhoD | 11 |
| RND1 | Rnd1 | 12 |
| ROBO1 | Robo1 | 3 |
| ROBO2 | Robo2 | 3 |
| ROBO3 | Robo3 | 11 |
| ROCK1 | ROCK | 18 |
| ROCK2 | ROCK | 2 |
| RRAS2 | Ras | 11 |
| SEMA3A/SEMAD | Sema3A | 7 |
| SEMA3B/SEMAA | Sema3A | 3 |
| SEMA3C/SEMAE | Sema3A | 7 |
| SEMA3D | Sema3A | 7 |
| SEMA3E/SEMAH | Sema3A | 7 |
| SEMA3F | Sema3A | 3 |
| SEMA4A/SEMAB | Sema4D | 1 |
| SEMA4B | Sema4D | 15 |
| SEMA4C/SEMAI | Sema4D | 2 |
| SEMA4D/SEMAJ | Sema4D | 9 |
| SEMA4F | Sema4D | 2 |
| SEMA4G | Sema4D | 10 |
| SEMA5A | Sema5 | 5 |
| SEMA5B | Sema5 | 3 |
| SEMA6A | Sema6 | 5 |
| SEMA6B | Sema6 | 19 |
| SEMA6C | Sema6 | 1 |
| SEMA6D | Sema6 | 15 |
| SEMA7A | Sema7A | 15 |
| SLIT1 | Slit1 | 10 |
| SLIT2 | Slit2 | 4 |
| SLIT3 | Slit3 | 5 |
| SRGAP1 | srGAP | 12 |
| UNC5A | UNC5 | 5 |
| UNC5B | UNC5 | 10 |
| UNC5C | UNC5 | 4 |
| UNC5D | UNC5 | 8 |
| CFL1/CFL | Cofilin | 11 |
| EFNA2/EPLG6 | Ephrin-A | 19 |
| EFNA3/EPLG3 | Ephrin-A | 1 |
| EFNA4/EPLG4 | Ephrin-A | 1 |
| EFNB3 | Ephrin-B | 17 |
| EPHB3/ETK2 | EphB | 3 |
| FES | Fes | 15 |
| MAPK3 | ERK | 16 |
| PLXNB2 | Plexin B | 22 |
| RAC3 | Rac | 17 |
| RRAS | Ras | 19 |

The invention will be further described in the following examples, which do not limit the scope of the invention described in the claims.

### EXAMPLES

### Example 1

### Identifying Sequence Polymorphisms that can Predict Parkinson's Disease

The following was performed to determine whether or not common variations in genes that encode polypeptides involved in the axon guidance pathway predispose to humans to PD. First, the Kyoto Encyclopedia of Genes and Genomes (KEGG) (Kanehisa, Trends Genet. 13:375 (1997); Kanehisa and Goto, Nucleic Acids Res. 28:27 (2000); and Kanehisa et al., Nucleic Acids Res. 34:D354 (2006)) was consulted. The KEGG pathway database is a bioinformatics resource that provides wiring diagrams of molecular interactions, reactions, and relations. There are several hundred pathways in KEGG related to Homo sapiens and diseases. This includes a detailed summary of the axon guidance pathway (World Wide Web at “genome.jp/dbget-bin/www_bget?path:hsa04360”). All of the genes that encoded polypeptides within the pathway were identified via Entrez Gene (World Wide Web at ncbi.nlm.nih.gov/entrez/query.fcgi?db=Gene). The UniGene database (World Wide Web at “ncbi.nlm.nih.gov/entrez/query.fcgi?db=unigene”) was used to determine which of the genes were expressed in human brain. The whole-genome association dataset for PD (Maraganore et al., Am. J. Hum. Genet. 77:685 (2005)) was used to identify those SNPs that were genotyped in brain-expressed, axon guidance pathway genes. Briefly, the whole-genome association dataset contains 198,345 genomic SNPs that were individually genotyped, uniformly spaced (1 per 12 kb average gap distance), and informative in 443 sibling pairs discordant for PD. This included 1,460 SNPs within 117 axon guidance pathway genes expressed in the brain.

All statistical tests were two-tailed, and considered significant at the conventional alpha level of 0.05. All statistical analyses were performed in SAS v. 9.1 (SAS Institute Inc., Cary, N.C.) or S-Plus v. 7 (Insightful Corp., Seattle, Wash.). Two phenotypes and three outcomes were considered of interest. For the first phenotype, PD susceptibility, the goal was to identify SNPs that predicted risk of PD (first outcome of interest). For the second phenotype, PD age at onset, the goal was to identify SNPs that predicted survival free of PD (second outcome of interest) or the reported age at onset of PD (third outcome of interest). A goal was to identify joint action models of SNPs from the axon guidance pathway that predicted each of the three outcomes.

For the first outcome, conditional logistic regressions stratified on sibship were used to examine associations of the SNPs with PD susceptibility while adjusting for age and gender (Breslow and Day, IARC Sci. Publ. 32:5 (1980)). For each SNP, odds ratios (ORs), 95% confidence intervals, and P values were calculated. Goodness-of-fit was assessed through measuring concordance and visually through histograms of predicted probabilities (Harrell et al., Stat. Med. 15:361 (1996)). The overall odds ratios were estimated by categorizing the predicted probability of PD from the model into four groups (<0.25, 0.25-0.50, 0.50-0.75, and >0.75), and then calculating the odds ratios for each group relative to the <0.25 group. A likelihood ratio test was used to assess the significance of the overall model, and a 95% bias-corrected bootstrap CI was calculated for the associated p value using 10,000 re-samples.

For the second outcome, Cox proportional hazards models were used to test for associations of the SNPs with survival free of PD (Cox, J. R. Stat. Soc. [Ser. B] 34:187 (1972)). For each SNP, hazard ratios (HRs), 95% confidence intervals, and P values were calculated. Concordance was again calculated for the proportional hazards models, and Kaplan-Meier plots of categorized scores predicting risk of PD were generated to provide visual gauges for goodness-of-fit (Kaplan and Meier, J. Am. Stat. Assoc. 53:457 (1958)). Hazard ratios were calculated for risk groups categorized at the quartiles, using the lowest risk group as reference. A likelihood ratio test was used to assess the significance of the overall model, and a 95% bias-corrected bootstrap CI was calculated for the associated p value using 10,000 re-samples.

For the third outcome, the reported age at onset of PD using multiple regression models was predicted (Draper and Smith, Applied Regression Analysis (Wiley, New York, ed. 2, 1981)). Goodness-of-fit was described through the model R² values and plots of the predicted vs. observed ages at onset. An F test was used to assess the significance of the overall model, and a 95% bias-corrected bootstrap CI was calculated for the associated p value and the R² using 10,000 re-samples. Assumptions were tested throughout. Only the linear regression models required a data transformation; age at onset-squared was used as the outcome to meet the required normality assumption. Tests of linkage disequilibrium (LD) in unaffected siblings was performed for the SNPs in the final models for the three outcomes using LDSELECT v. 1.0 (2004 by Deborah A. Nickerson, Mark Rieder, Chris Carlson, Qian Yi, University of Washington) with a threshold R² of 0.80.

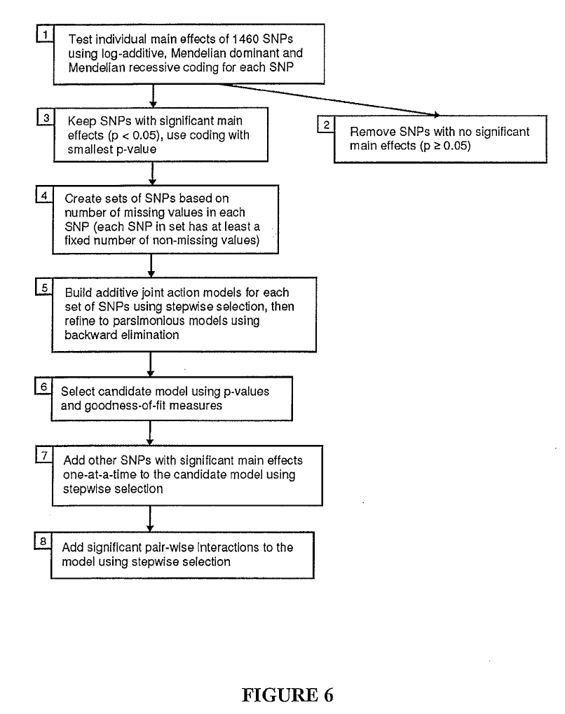

The following scheme was used to develop models for each outcome (FIG. 6). Since the modes of expression of the alleles in the SNPs of interest were not known, each SNP was analyzed using three coding schemes: log-additive, Mendelian dominant, and Mendelian recessive (Step 1). Subsequent analyses was simplified by removing from further consideration those SNPs with no significant main effects in any coding scheme (Step 2). Removing those SNPs was a conservative approach, potentially biasing tests towards the null hypotheses, since the SNPs were prevented from possibly entering the joint action models after adjustment for other variables. The remaining SNPs were generally coded using the schemes which produced the smallest p values, since these provided best estimates of the modes of expression in the data (Step 3). For each outcome, multiple sets of SNPs were created, where each set contained only SNPs with at least a certain number of non-missing values (Step 4). This was done to address issues due to missing values.

While most SNPs had fairly complete data, others had missing values from substantial numbers of subjects: up to 18% of cases, and up to 32% of case-sib pairs. An approach was chosen where candidate models were constructed using sets of SNPs with fairly complete data (effective sample sizes close to the maximum 443) to explain as much of the outcomes as possible and then checked to see if adding other SNPs on top of the candidate models would contribute significantly. The candidate models for each set were constructed using standard automated procedures (Step 5). A final candidate model was selected for each outcome based on significance and goodness-of-fit (Step 6). Other SNPs, which were significant given the candidate models (Step 7) and had significant pair-wise interactions (Step 8), were then added.

To compare the significance of axon guidance pathway SNP models to the significance of randomly selected genomic SNP models, 4,000 models were constructed for each outcome by randomly selecting the appropriate number of SNPs from the entire available data set. The distributions of the test statistics from those models and the values of the test statistics from final models were then plotted.

Validation with a Second Whole-Genome Association Dataset

A second whole-genome association study of PD is described elsewhere (Fung et al., Lancet Neurol., 5: 911-916 (2006)). That study included 276 patients with PD and 276 neurologically normal and unrelated controls. The samples used for that study were derived from the NINDS Neurogenetics repository hosted by the Coriell Institute for Medical Research. There were 408,803 SNPs individually genotyped, and call rates and Hardy-Weinberg equilibrium p-values were described elsewhere (Elbaz et al., Lancet Neurol., 5:917-923 (2006)). The individual level data for that study were downloaded from the Coriell Institute website, and SNPs were identified in that secondary dataset that were assigned to the genes represented by SNPs in each of the predictive genetic models for the primary dataset (same genes and outcomes, different SNPs and samples). The same statistical methods were then employed to construct predictive genetic models for the same outcomes in the secondary dataset, with two exceptions. First, because the secondary dataset employed unrelated controls that were not individually matched, unconditional logistic regression analyses were performed instead of conditional logistic regression analyses for the PD susceptibility outcome. Second, the age at onset of PD analyses in the secondary dataset was restricted to exclude subjects with ages younger than those reported in the primary dataset, which resulted in the exclusion of one subject whose age at onset was reportedly 13 years.

Validation with a Gene Expression Profiling Dataset

An available gene expression profiling dataset was explored to determine if there was convergence of those functional data with the genetic association data and models (Papapetropoulos et al., Gene Expression, 13:205-215 (2006)). That study included multiregional gene expression data from postmortem brain specimens from 22 PD cases and 23 normal aged brain donors and represents the most comprehensive expression profiling study of PD to date (largest numbers of subjects, brain regions studied, and genes assayed). Very strict RNA quality control criteria were used. Data derived from Affymetrix Human Genome U133 Plus 2.0 GeneChip arrays, which included probe set data for 126 of the 128 brain-expressed axon guidance pathway genes that were initially identified were analyzed. Probe set data for the substantia nigra, putamen, and caudate regions were analyzed using methods and criteria similar to those described elsewhere (Papapetropoulos et al., Gene Expression, 13:205-215 (2006)).

This and the following three paragraphs contain information regarding the study design, expression values calculations, and array normalization methods for the published study. Briefly, postmortem brain tissue was obtained from University of Miami/NPF Brain Endowment Bank donors diagnosed with neuropathologically confirmed PD (Hughes et al., J. Neurol. Neurosurg. Psychiatry, 55(3):181-184 (1992)) (n=22) or aged individuals with no history or pathological diagnosis of neurologic or psychiatric disease (n=23). Detailed demographics of the cohort is described elsewhere (Papapetropoulos et al., Gene Expression, 13(3):205-215 (2006)). All patients were carefully characterized for clinical phenotype, treatment, and agonal state. Regional samples of postmortem brain were taken from frozen coronal blocks based on surface and cytoarchitectural landmarks after controlling for important determinants of RNA quality (postmortem interval, brain pH).

Whole tissue from the substantia nigra, putamen, and caudate was used to conduct gene microarray experiments. Total RNA isolation was performed using a TriZol method and RNeasy columns, and labeled cRNA was prepared according to the manufacturer's protocol (Affymetrix, Santa Clara, Calif.). Total RNA from each sample was used to prepare biotinylated target RNA, following the manufacturer's recommendations (See, e.g., World Wide Web at “affymetrix.com/support/technical/manual/expression_manual.affx”). The target cDNA generated from each sample was processed as per manufacturer's recommendation using an Affymetrix GeneChip Instrument System (See, e.g., World Wide Web at “affymetrix.com/support/technical/manual/expression_manual.affx”). The samples were checked for evidence of degradation and integrity, A260/A280>1.9 and 28S/18S>1.6, (2100 Bioanalyzer; Agilent Technologies, Palo Alto, Calif.). The Human Genome U133 Plus 2.0 GeneChips were used (See, e.g., World Wide Web at “affymetrix.com/products/arrays/specific/hgu133plus.affx”). Microarray quality control parameters included the following: background, noise (RawQ), consistent number of genes detected as present across arrays, consistent scale factors, and consistent β-actin and glyceraldehyde-3-phosphate dehydrogenase 5′/3′ signal ratios.

The gene expression from three different brain regions in PD patients and their age matched normal subjects were analyzed. After checking for microarray quality control, the following number of samples were used in the analysis of genes in the constructed models of axonal guidance: for substantia nigra 16 Parkinson's disease patients vs. 8 controls, for putamen 13 Parkinson's disease patients vs. 7 controls, and for caudate nucleus 14 Parkinson's disease patients vs. 9 controls (22 cases and 23 controls total).

Genes were selected on the basis of “present calls” by Microarray Analysis Suite 5.0. For a gene to be included, it had to be present (detectable) in at least 75% of the subjects in at least 1 of the 2 groups (Parkinson's disease patients and controls) to reduce the chances of false-positive findings. Expression data were analyzed using Genesis (GeneLogic, Gaithersburg, Md.) and AVADIS software (Strand Genomics, Redwood City, Calif.). Following normalization, one-way analysis of variance was performed for each gene to identify statistically significant gene expression changes. Two criteria were used to determine whether a gene was differentially expressed: p value ≦0.05 and fold-change (FC) of ±1.3.

For the differential expression analyses of the present study, probe sets were identified from within the original dataset that were assigned to the axon guidance pathway genes of interest (those represented by SNPs in either of the three predictive genetic models in the primary whole-genome association dataset). For each probe set, expression for cases and controls were compared, in each of the three nigrostriatal regions. A probe set was considered informative in a given region if it was expressed in at least 75% of cases or 75% of controls. Although differential expression of a single probe set is most of the times enough to characterize a gene as differentially expressed (multiple polyadenylation sites are represented on Affymetrix gene chips to account for multiple gene transcripts), for the present study a more conservative definition of differential gene expression than for the original study was employed. A gene was defined as differentially expressed in a given region if at least one accurate-type (at) probe set assigned to the gene had a t-test p value <0.05 and absolute value of the fold expression ≧1.3. Alternatively, a gene was defined as differentially expressed in a given region if at least 30% of accurate-type (at) or cross reacting-type (x_at, s_at) probe sets assigned to the gene had t-test p values <0.05 and absolute values of the fold expression ≧1.3. These definitions weigh the significance of cross-reactive type probe sets lower than accurate-type probe sets because they have less specificity, and also account for the possibility that multiple probe sets for the same gene may not provide concordant gene expression measurements (Elbez et al., BMC Genomics, 7:136 (2006)).

A sensitivity analysis was also performed where a gene was defined as differentially expressed in a given region if at least one probe set of any type assigned to the gene had a t-test p value <0.05 and absolute value of the fold expression ≧1.3. This was consistent with the differential expression analyses performed for the original study. Finally, a second sensitivity analysis was performed whereby the same CEL file data as for the original gene expression profiling study was employed. The data were re-normalized and re-analyzed using a second standard software package (GeneSpring, Agilent Technologies). That normalization procedure obtained informative results for all probe sets and in all 45 genes. Briefly, gene chip raw Affymetrix data in the form of CEL files were uploaded in GeneSpring v7.2 (Agilent Technologies), normalized with GC-RMA, and re-analyzed using the same statistical tests and criteria.

For the primary differential expression analyses, possible bias due to the number of informative probe sets per gene was tested by comparing the distributions for differentially expressed versus normally expressed genes using the Wilcoxon rank sum test. For the primary and two sensitivity analyses, the differential expression of genes was coded as increased or reduced if all probe sets assigned to the gene had the same direction of effect, or ambiguous if the probe sets had opposite directions of effect.

### Results

The available whole-genome association study dataset included 443 PD cases and 443 unaffected sibling controls (Tier 1 sample). Details regarding the demographic and clinical characteristics of these subjects is described elsewhere (Maraganore et al., Am. J. Hum. Genet. 77:685 (2005)). The median age at onset of PD among the cases was 61 years (range 31-94). Details regarding the SNP markers genotyped, including call rates, Hardy-Weinberg equilibrium estimations, and re-genotyping concordance rates are described elsewhere (Maraganore et al, Am. J. Hum. Genet. 77:685 (2005)). A detailed listing of SNPs within axon guidance pathway genes expressed in the brain is provided in Table 4. Table 4 contains sequences that can be used to assess a human's nucleic acid for the presence of a polymorphism set forth in Table 1, 2, 3, or 4.

Of the 1,376 SNPs within brain-expressed genes of the KEGG axon-guidance pathway, 183 SNPs (13.3%) were individually associated with susceptibility to PD. Table 1 contains results for the final model produced by running SNPs through the multi-stage process to predict PD susceptibility. This model used data from 442 matched PD patients/sibling controls (1 pair was missing data on one or more SNPs). To determine the significance of the pathway, rather than individual SNPs, the P value for the overall model was assessed. In this case, the model had an overall P value of 4.64×10⁻³⁸.

TABLE 1
Axon-guidance SNPs predicting Parkinson's disease susceptibility.

| Gene* (rs ID or ss ID), | Odds Ratio | | | | Odds Ratio | | |
|---|---|---|---|---|---|---|---|
| Final Coding Scheme^{†} | (95% CI)** | | P value** | Interaction* | (95% CI)** | | P value** |
| CDC42 (rs12740705), d | 0.16 | (0.06-0.41) | 1.3 × 10⁻⁰⁴ | DCC*PAK4 | 0.02 | (0.002-0.19) | 6.0 × 10⁻⁰⁴ |
| CHP (rs6492998), d | 0.21 | (0.09-0.47) | 1.6 × 10⁻⁰⁴ | EPHA4*FYN | 0.16 | (0.05-0.53) | 2.8 × 10⁻⁰³ |
| DCC (rs17468382), d | 0.07 | (0.01-0.67) | 2.1 × 10⁻⁰² | EPHA4*PAK7 | 0.26 | (0.11-0.62) | 2.7 × 10⁻⁰³ |
| EFNA5 (ss46558780), d | 0.80 | (0.29-2.21) | 6.7 × 10⁻⁰¹ | EPHB2*EFNA5 | 5.25 | (2.10-13.13) | 4.0 × 10⁻⁰⁴ |
| EPHA4 (rs13386128), a | 16.29 | (5.95-44.59) | 5.6 × 10⁻⁰⁸ | FYN*RRAS2 | 0.04 | (0.003-0.50) | 1.3 × 10⁻⁰² |
| EPHB1 (rs2030737), d | 2.60 | (1.23-5.51) | 1.2 × 10⁻⁰² | FYN*SLIT3 | 4.52 | (1.32-15.47) | 1.6 × 10⁻⁰² |
| EPHB2 (rs10917325), a | 0.11 | (0.05-0.28) | 1.6 × 10⁻⁰⁶ | MRAS*SLIT3 | 0.18 | (0.08-0.41) | 5.9 × 10⁻⁰⁵ |
| FYN (rs6910116), r | 59.95 | (5.01-717.33) | 1.2 × 10⁻⁰³ | PAK7*CHP | 2.94 | (1.23-7.01) | 1.5 × 10⁻⁰² |
| GNAI3 (rs6692804), r | 0.07 | (0.01-0.32) | 6.3 × 10⁻⁰⁴ | SEMA5A*RAC2 | 4.95 | (1.37-17.91) | 1.5 × 10⁻⁰² |
| GSK3B (rs16830689), r | 0.09 | (0.02-0.44) | 2.9 × 10⁻⁰³ | UNC5C₁*DCC | 6.89 | (1.20-39.51) | 3.0 × 10⁻⁰² |
| MRAS (rs4678260), la | 0.97 | (0.57-1.65) | 9.1 × 10⁻⁰¹ | | | | |
| NTNG1 (rs11185076), r | 0.02 | (0.001-0.32) | 5.7 × 10⁻⁰³ | | | | |
| PAK4 (rs17641276), r | 9.55 | (3.73-24.43) | 2.5 × 10⁻⁰⁶ | | | | |
| PAK7 (rs2072952), a | 1.70 | (0.75-3.90) | 2.1 × 10⁻⁰¹ | | | | |
| PLXNA2 (rs6656034), a | 2.92 | (1.75-4.87) | 4.0 × 10⁻⁰⁵ | | | | |
| PLXNC1 (rs2068435), a | 0.51 | (0.25-1.02) | 5.5 × 10⁻⁰² | | | | |
| PPP3CA (rs2044041), d | 5.72 | (2.84-11.51) | 1.1 × 10⁻⁰⁶ | | | | |
| RAC2 (rs739043), r | 0.16 | (0.069-0.39) | 4.8 × 10⁻⁰⁵ | | | | |
| RRAS2 (rs2970332), d | 8.78 | (2.45-31.44) | 8.5 × 10⁻⁰⁴ | | | | |
| SEMA5A (ss46559487), d | 0.22 | (0.10-0.49) | 2.0 × 10⁻⁰⁴ | | | | |
| SLIT3 (rs9688032), d | 17.46 | (4.61-66.07) | 2.5 × 10⁻⁰⁵ | | | | |
| UNC5C₁ (rs11097458), a | 2.25 | (1.41-3.61) | 7.2 × 10⁻⁰⁴ | | | | |
| UNC5C₂ (rs4444836), d | 0.38 | (0.17-0.85) | 1.8 × 10⁻⁰² | | | | |

*Subscript is used to identify individual SNPs in interactions.
**Results adjusted for age and gender.
^{†}a = log additive, d = Mendelian dominant, r = Mendelian recessive.

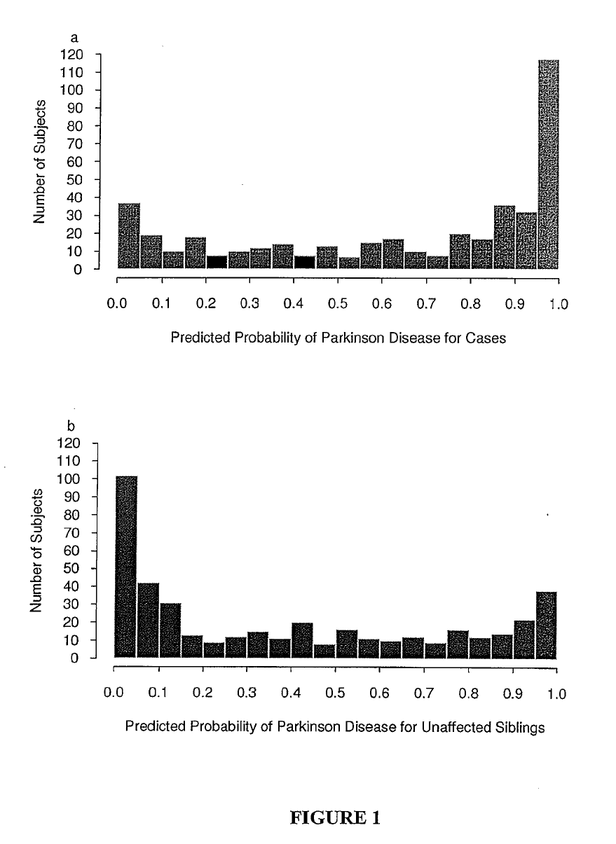

This model significantly predicted whether or not an individual was a case or unaffected sibling. The predicted probabilities of PD were high (towards 1) for most of the cases, and low (towards 0) for most of the unaffected siblings (FIG. 1). Indeed, 35% of the cases had predicted probabilities above 0.9, and 34% of unaffected siblings had predicted probabilities below 0.1. The model did not completely distinguish the two groups—some cases had low predicted probabilities, and some controls had high predicted probabilities (FIG. 1). The concordance for the model was about 0.70, indicating good but not near complete agreement between predicted and observed case/sibling status.

Of the 1,376 SNPs, 175 (12.7%) were individually associated with age at onset (hazard function) using Cox proportional hazards models. Table 2 contains results for the final proportional hazards model produced by running SNPs through the multi-stage process to predict PD age at onset. This model used data from 400 PD patients (43 patients were missing data on one or more SNPs). In this case, the model had an overall P value of 5.43×10⁻⁴⁸. The model was not significant at predicting age at study of the matched sibling controls (P=0.73). This last finding suggests that the model predicts age at onset (hazard function) of PD, not age in general, and that the model is specific for PD cases.

TABLE 2
Axon-guidance SNPs predicting survival free of Parkinson's disease.

| Gene* (rs ID or ss ID), | Hazard Ratio | | | Hazard Ratio | |
|---|---|---|---|---|---|
| Final Coding Scheme^{†} | (95% CI) | P value | Interaction* | (95% CI) | P value |
| ABLIM2 (rs10008860), r | 1.68 (1.30-2.18) | 7.1 × 10⁻⁰⁵ | DCC₁*NTNG1 | 2.91 (1.30-6.52) | 9.3 × 10⁻⁰³ |
| DCC₁ (rs16954702), a | 0.78 (0.38-1.57) | 4.8 × 10⁻⁰¹ | EPHA8*EPHB1₁ | 0.12 (0.02-0.76) | 2.5 × 10⁻⁰² |
| DCC₂ (rs4940245), d | 0.46 (0.33-0.64) | 4.5 × 10⁻⁰⁶ | EPHB1₁*DPYSL2 | 2.89 (1.26-6.62) | 1.2 × 10⁻⁰² |
| DPYSL2 (rs327234), r | 0.30 (0.13-0.66) | 2.7 × 10⁻⁰³ | PAK6*NFATC4 | 1.46 (1.08-1.97) | 1.3 × 10⁻⁰² |
| EPHA8 (ss46536672), r | 1.81 (0.38-8.68) | 4.6 × 10⁻⁰¹ | ROBO1₂*PLXNC1₂ | 0.43 (0.25-0.75) | 2.9 × 10⁻⁰³ |
| EPHB1₁ (rs1554675), r | 1.08 (0.71-1.64) | 7.1 × 10⁻⁰¹ | ROBO2*DCC₂ | 1.82 (1.18-2.82) | 7.2 × 10⁻⁰³ |
| EPHB1₂ (rs36183), r | 1.65 (1.28-2.13) | 1.1 × 10⁻⁰⁴ | ROBO2*SEMA3A | 0.40 (0.26-0.63) | 4.7 × 10⁻⁰⁵ |
| KRAS (rs11047902), d | 0.62 (0.48-0.80) | 3.0 × 10⁻⁰⁴ | SEMA3A*RRAS2 | 3.39 (1.66-6.90) | 7.8 × 10⁻⁰⁴ |
| NFATC2 (ss46555247), a | 1.47 (1.20-1.79) | 1.6 × 10⁻⁰⁴ | SLIT3₂*NTNG1 | 0.47 (0.28-0.79) | 3.9 × 10⁻⁰³ |
| NFATC4 (rs2295298), a | 1.29 (1.04-1.60) | 2.3 × 10⁻⁰² | SLIT3₁*SEMA3A | 0.57 (0.35-0.91) | 1.9 × 10⁻⁰² |
| NTNG1 (rs12731259), d | 2.41 (1.55-3.75) | 9.1 × 10⁻⁰⁵ | | | |
| PAK3 (rs10521534), r | 5.89 (2.26-15.31) | 2.8 × 10⁻⁰⁴ | | | |
| PAK6 (ss46552269), a | 0.46 (0.30-0.70) | 2.6 × 10⁻⁰⁴ | | | |
| PLXNC1₁ (rs2068435), r | 3.27 (1.61-6.65) | 1.1 × 10⁻⁰³ | | | |
| PLXNC1₂ (rs3803071), a | 1.45 (1.22-1.72) | 2.9 × 10⁻⁰⁵ | | | |
| PPP3CA (rs1506801), a | 1.58 (1.34-1.85) | 3.6 × 10⁻⁰⁸ | | | |
| ROBO1₁ (rs6769328), d | 0.49 (0.35-0.69) | 2.8 × 10⁻⁰⁵ | | | |
| ROBO1₂ (ss46540991), r | 1.24 (0.64-2.41) | 5.2 × 10⁻⁰¹ | | | |
| ROBO2 (rs17015294), d | 0.63 (0.42-0.93) | 2.0 × 10⁻⁰² | | | |
| ROCK2 (rs6721445), d | 0.26 (0.12-0.60) | 1.4 × 10⁻⁰³ | | | |
| RRAS2 (rs1431374), r | 0.36 (0.23-0.59) | 3.2 × 10⁻⁰⁵ | | | |
| SEMA3A (ss46545340), d | 1.13 (0.80-1.60) | 5.0 × 10⁻⁰¹ | | | |
| SEMA3D (rs12176601), d | 1.32 (1.06-1.64) | 1.3 × 10⁻⁰² | | | |
| SEMA3E (rs215285), r | 1.98 (1.45-2.71) | 1.8 × 10⁻⁰⁵ | | | |
| SEMA5A (rs3822787), r | 5.28 (2.47-11.3) | 1.8 × 10⁻⁰⁵ | | | |
| SLIT3₁ (rs1432899), d | 2.21 (1.60-3.06) | 1.7 × 10⁻⁰⁶ | | | |
| SLIT3₂ (rs7715777), d | 1.21 (0.80-1.83) | 3.6 × 10⁻⁰¹ | | | |
| SLIT3₃ (ss46557969), r | 0.54 (0.42-0.71) | 5.2 × 10⁻⁰⁶ | | | |
| SRGAP1 (rs1840664), d | 2.30 (1.69-3.12) | 9.4 × 10⁻⁰⁸ | | | |
| SRGAP2 (rs6762693), a | 0.66 (0.54-0.82) | 1.2 × 10⁻⁰⁴ | | | |
| UNC5A (rs539822), d | 2.51 (1.23-5.09) | 1.1 × 10⁻⁰² | | | |
| UNC5D (rs13256961), a | 1.12 (0.96-1.31) | 1.6 × 10⁻⁰¹ | | | |

*Subscript is used to identify individual SNPs in interactions.
^{†}a = log additive, d = Mendelian dominant, r = Mendelian recessive.

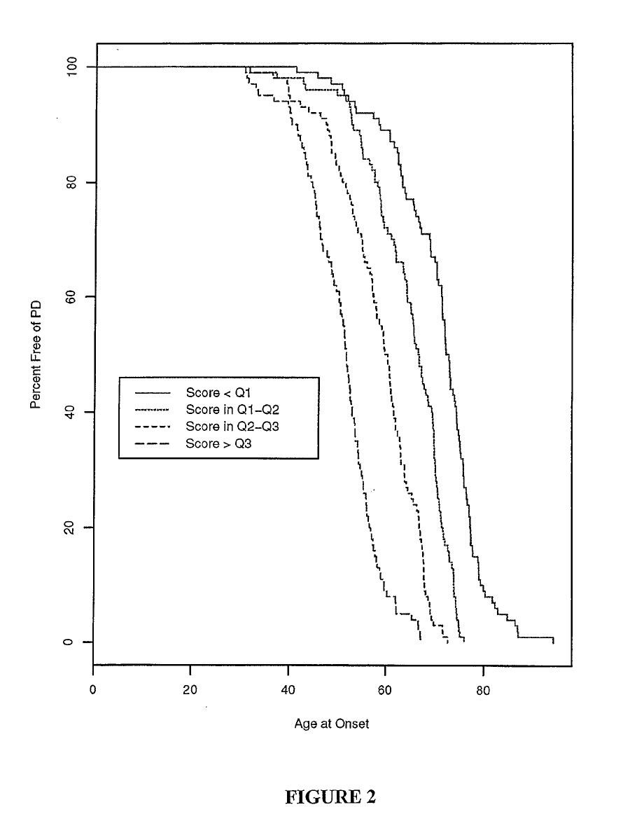

FIG. 2 uses a Kaplan-Meier plot to describe the results of the model. The groups were formed by calculating a risk score for each PD patient using the equation from the proportional hazards model, then categorizing the score at the 25^{th} (Q1), 50^{th} (Q2), and 75^{th} (Q3) percentiles. The survival curves separated nicely right from the earliest ages of onset. By age 60, only 9% of PD patients in the predicted highest risk group were still free of PD, whereas 89% of PD patients in the predicted lowest risk group were free of PD. By age 70, none of the PD patients in the predicted highest risk group were still free of PD, whereas 66% of PD patients in the predicted lowest risk group were free of PD. The median ages at onset for each group, from lowest risk group to highest risk group, were 72.1, 66.5, 59.7, and 51.7, a difference of more than 20 years from lowest to highest. The concordance for this model was 0.76.

Of the 1,376 SNPs, 160 (11.6%) were individually associated with age at onset using linear regression models. Table 3 contains results for the final model produced by running SNPs through the multi-stage process to predict PD age at onset-squared. This model used data from 395 PD patients (48 patients were missing data on one or more SNPs). In this case, the model had an overall P value of 1.68×10⁻⁵¹. The set of SNPs was not significant at predicting age at study-squared of the matched sibling controls (P=0.34). This last finding suggests that the model predicts age at onset of PD, not age in general, and that the model is specific for PD cases.

TABLE 3
Axon-guidance SNPs predicting age at onset of Parkinson's disease.

| | Regression | | | | | | |
|---|---|---|---|---|---|---|---|
| Gene* (rs ID or ss ID), | coefficient | | | | Regression coefficient | | |
| Final Coding Scheme^{†} | (standard error) | | P value | Interaction* | (standard error) | | P value |
| (Intercept) | 2177.35 | (643.06) | 8.0 × 10⁻⁰⁴ | ABLIM2₁*CXCR4 | 3399.00 | (993.00) | 7.0 × 10⁻⁰⁴ |
| ABL1 (rs17147263), r | −1845.63 | (610.17) | 2.7 × 10⁻⁰³ | ABLIM2₁*NFATC4 | 466.16 | (169.44) | 6.3 × 10⁻⁰³ |
| ABLIM2₁ (rs10008860), r | −919.68 | (247.79) | 2.4 × 10⁻⁰⁴ | CDC42*CXCR4 | −2274.37 | (976.73) | 2.0 × 10⁻⁰² |
| ABLIM2₂ (rs2386113), d | 2385.58 | (530.76) | 9.7 × 10⁻⁰⁶ | DPYSL2*DCC | 548.09 | (236.29) | 2.1 × 10⁻⁰² |
| CDC42 (rs2056974), d | −366.15 | (212.79) | 8.6 × 10⁻⁰² | DPYSL2*PLXNC1 | 513.80 | (203.78) | 1.2 × 10⁻⁰² |
| CXCR4 (rs2734871), r | 4797.71 | (1169.33) | 5.1 × 10⁻⁰⁵ | EFNA5₁*CXCR4 | −3066.66 | (750.24) | 5.5 × 10⁻⁰⁵ |
| DCC (rs1367637), r | −514.16 | (105.45) | 1.7 × 10⁻⁰⁶ | EFNA5₁*NFATC4 | 383.76 | (182.88) | 3.7 × 10⁻⁰² |
| DPYSL2 (rs327234), r | −287.03 | (188.67) | 1.3 × 10⁻⁰¹ | EPHA4*PPP3CA | −384.13 | (105.22) | 3.0 × 10⁻⁰⁴ |
| EFNA5₁ (rs152562), d | −710.18 | (243.20) | 3.7 × 10⁻⁰³ | NFATC2₁*NFATC4 | −473.13 | (156.98) | 2.8 × 10⁻⁰³ |
| EFNA5₂ (rs3797510), r | 502.49 | (307.99) | 1.0 × 10⁻⁰¹ | NFATC2₂*ROBO1₂ | 739.45 | (274.32) | 7.4 × 10⁻⁰³ |
| EPHA4 (rs2303901), a | −28.75 | (93.95) | 7.6 × 10⁻⁰¹ | NFATC4*PAK6 | −415.00 | (117.66) | 4.8 × 10⁻⁰⁴ |
| EPHB1 (ss46540509), a | −386.45 | (65.46) | 8.8 × 10⁻⁰⁹ | PAK1*NFATC2₁ | 301.08 | (139.99) | 3.2 × 10⁻⁰² |
| NFATC2₁ (ss46555246), r | 737.17 | (350.16) | 3.6 × 10⁻⁰² | PPP3CA*SLIT3₂ | 645.01 | (154.72) | 3.9 × 10⁻⁰⁵ |
| NFATC2₂ (ss46555247), a | 90.19 | (169.86) | 6.0 × 10⁻⁰¹ | PPP3CA*SRGAP1₂ | −560.10 | (206.16) | 6.9 × 10⁻⁰³ |
| NFATC4 (rs2295298), a | −305.84 | (180.30) | 9.1 × 10⁻⁰² | ROBO1₁*NFATC2₁ | −790.72 | (294.22) | 7.6 × 10⁻⁰³ |
| NTNG1 (rs4579786), r | 1579.13 | (539.98) | 3.7 × 10⁻⁰³ | ROBO1₁*UNC5D | 1536.78 | (383.22) | 7.5 × 10⁻⁰⁵ |
| PAK1 (rs503406), a | 168.89 | (79.54) | 3.4 × 10⁻⁰² | ROBO2*PLXNC1 | −588.78 | (160.31) | 2.8 × 10⁻⁰⁴ |
| PAK3 (rs10521534), r | −956.72 | (410.60) | 2.0 × 10⁻⁰² | ROBO2*PPP3CA | −299.35 | (139.63) | 3.3 × 10⁻⁰² |
| PAK6 (ss46552269), a | 836.89 | (166.71) | 8.5 × 10⁻⁰⁷ | ROBO2*SLIT3₂ | 493.18 | (199.90) | 1.4 × 10⁻⁰² |
| PAK7 (rs6086976), r | 456.37 | (247.31) | 6.6 × 10⁻⁰² | SEMA4D*NFATC2₂ | −421.48 | (121.09) | 5.7 × 10⁻⁰⁴ |
| PLXNC1 (rs10507035), a | 57.35 | (122.66) | 6.4 × 10⁻⁰¹ | SEMA5A₁*SEMA4D | 865.24 | (323.95) | 7.9 × 10⁻⁰³ |
| PPP3CA (rs1506801), a | 189.91 | (158.71) | 2.3 × 10⁻⁰¹ | SEMA6C*EFNA5₂ | 1497.93 | (550.81) | 6.9 × 10⁻⁰³ |
| ROBO1₁ (rs6769328), d | 319.29 | (169.17) | 6.0 × 10⁻⁰² | SEMA6C*ROBO2 | 712.21 | (184.45) | 1.4 × 10⁻⁰⁴ |
| ROBO1₂ (ss46540991), r | 118.28 | (188.93) | 5.3 × 10⁻⁰¹ | SEMA6C*SEMA5A₁ | 1275.38 | (429.55) | 3.2 × 10⁻⁰³ |
| ROBO2 (rs17015294), d | 232.75 | (182.73) | 2.0 × 10⁻⁰¹ | SLIT3₁*UNC5D | −3038.73 | (977.12) | 2.0 × 10⁻⁰³ |
| SEMA3E (rs215285), r | −416.74 | (128.40) | 1.3 × 10⁻⁰³ | SLIT3₂*UNC5D | −575.60 | (229.89) | 1.3 × 10⁻⁰² |
| SEMA4D (rs9969727), a | 297.51 | (78.41) | 1.8 × 10⁻⁰¹ | SRGAP2*SLIT3₂ | −446.03 | (179.54) | 1.3 × 10⁻⁰² |
| SEMA5A₁ (rs16882671), a | −2288.01 | (575.23) | 8.6 × 10⁻⁰⁵ | SRGAP2*SLIT3₃ | −397.10 | (165.82) | 1.7 × 10⁻⁰² |
| SEMA5A₂ (rs3822787), r | −1172.76 | (314.54) | 2.3 × 10⁻⁰⁴ | | | | |
| SEMA6C (rs4357530), r | −41.57 | (141.18) | 7.7 × 10⁻⁰¹ | | | | |
| SLIT2 (rs1355512), r | −482.36 | (192.17) | 1.3 × 10⁻⁰² | | | | |
| SLIT3₁ (rs11738097), r | 278.84 | (289.97) | 3.4 × 10⁻⁰¹ | | | | |
| SLIT3₂ (rs1432899), d | −736.37 | (193.67) | 1.7 × 10⁻⁰⁴ | | | | |
| SLIT3₃ (rs4868530), d | 398.81 | (112.49) | 4.5 × 10⁻⁰⁴ | | | | |
| SRGAP1₁ (rs10784373), d | 271.13 | (93.81) | 4.1 × 10⁻⁰³ | | | | |
| SRGAP1₂ (rs1840664), d | −247.09 | (174.00) | 1.6 × 10⁻⁰¹ | | | | |
| SRGAP2 (rs6762693), a | 518.98 | (124.81) | 4.1 × 10⁻⁰⁵ | | | | |
| UNC5D (ss46546978), a | −791.59 | (375.74) | 3.6 × 10⁻⁰² | | | | |

*Subscript is used to identify individual SNPs in interactions.
^{†}a = log additive, d = Mendelian dominant, r = Mendelian recessive.

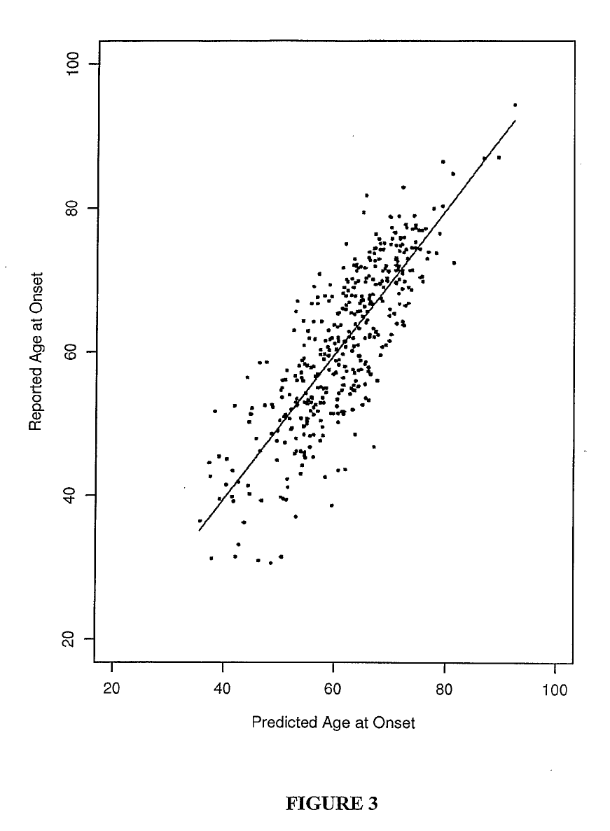

FIG. 3 shows a plot of predicted age at onset vs. reported age at onset to summarize the results of the model. The plot revealed a nice elliptical pattern, reflecting the model R² of 0.683. The model explained about 68% of the variability in age at onset of PD.

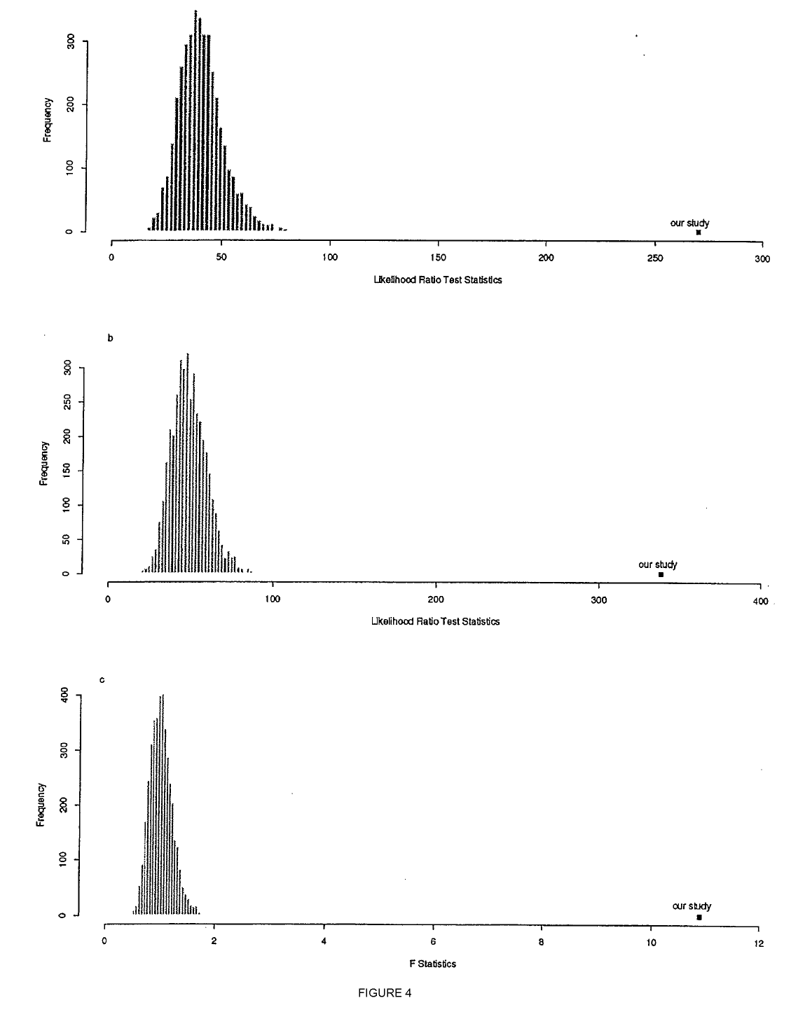

FIG. 4 shows the distributions of the test statistics from the models with randomly selected SNPs and the values of the test statistics from the final models for PD susceptibility (A), survival free of PD (B), and age at onset of PD (C). The test statistics from the final models were in every case much greater than those observed from the other models.

Other combinations of SNPs from the axon guidance pathway also performed well in predicting PD susceptibility and age at onset. Although the models provided herein revealed the best fit of the data, the results do not preclude other combinations of axon guidance pathway SNPs as significant predictors of PD. The SNPs in the final models selected exhibited no significant LD in unaffected siblings.

TABLE 4
Axon-guidance SNPs.

| | | | | | | | | |
|---|---|---|---|---|---|---|---|---|
| | | | | | Contig | Ref.* | Alt | |
| ID | Gene Name | rsID | ssID | Chr | Position | Allele | Allele | Sequence |

| | | | | | | | | |
|---|---|---|---|---|---|---|---|---|
| 1 | EPHA2 | 3754334 | 24245700 | 1 | 16197073 | G | A | AGCAGGCTGTAGGCNATGCGCTTCTGGTG |
| 2 | EPHA2 | 3768294 | 23160860 | 1 | 16201482 | G | A | TGGATGATTGACTCNGCTGCCCCGTGCCT |
| 3 | EPHA2 | 3820609 | 23160923 | 1 | 16212951 | T | G | CAAAAGCCAGAAACNCCTGACGAATTTCA |
| 4 | EPHA2 | 12408813 | 24245927 | 1 | 16233975 | G | A | GTGCCGGGCATACANTGGTGCCCAGTAAA |
| 5 | CDC42 | • | 46534404 | 1 | 22125615 | C | T | CCGCCTTCAGTGCANCTAAAAGCTAGGAA |
| 6 | CDC42 | • | 46536668 | 1 | 22140557 | C | G | CTTAGTTTCTGAGTNCAGTCTTCTTTGCA |
| 7 | CDC42 | 2056974 | 24249108 | 1 | 22154400 | T | A | GAATGTAGGAATTCNAAACTTTGTGCTTT |
| 8 | CDC42 | 2268179 | 23846491 | 1 | 22160091 | G | T | TTCTCTAGATATGGNTTGCTTTGAGGGAA |
| 9 | CDC42 | 12740705 | 24249196 | 1 | 22166419 | C | T | TTTTATAGTTAAAGNGATGTTCTTCAAGC |
| 10 | CDC42 | 6672792 | 24249250 | 1 | 22174007 | A | G | TATTTTCCTACCTTNTGGATCACTTCCTT |
| 11 | EPHA8 | • | 46536672 | 1 | 22630879 | G | A | TATCAGACGGGACANTCTTAGACCCTTCT |
| 12 | EPHA8 | • | 46536671 | 1 | 22647539 | T | A | CGCCTCTCCTGCACNGCTGCTTGTTGAGT |
| 13 | EPHA8 | 10917268 | 24152512 | 1 | 22683124 | C | T | GAAGGGTTATGCACNAGGACGTTAGATAT |
| 14 | EPHB2 | 12562997 | 23847502 | 1 | 22787777 | G | A | TTCCCTCAATGATGNCGACGTGGCCCCCT |
| 15 | EPHB2 | • | 46536667 | 1 | 22795658 | G | A | TCAGCCACTCAAAGNCAATAATAATAGTA |
| 16 | EPHB2 | 17487259 | 24250674 | 1 | 22808469 | G | A | CGCGGCATGTTTGCNCCGCTGACACTCTG |
| 17 | EPHB2 | 6426763 | 23847596 | 1 | 22814067 | G | A | GAAGGATGCCCAGANTGAGACAGGTTGGA |
| 18 | EPHB2 | 4654814 | 24250766 | 1 | 22839727 | C | T | GCTCTCCTGAGAAANATCGACATGCGAGA |
| 19 | EPHB2 | 12028458 | 23847693 | 1 | 22847678 | C | G | CTTTCTTCATCTATNCATTGGGATTCPTTT |
| 20 | EPHB2 | 12724658 | 23167980 | 1 | 22861055 | G | C | TCTAAGAGGATGTGNTTTGAAAAGATGCA |
| 21 | EPHB2 | 12407525 | 23847866 | 1 | 22895127 | A | C | CTGACTCCTCTTCANGTCTGTATTATAAT |
| 22 | EPHB2 | 4655130 | 24251137 | 1 | 22921300 | G | A | TATGTGGAACAACCNTTGCAGATTTCAGA |
| 23 | EPHB2 | 16827672 | 23847999 | 1 | 22927990 | G | A | GGCCAGCCATTGAGNTTTTGAGGATCACC |
| 24 | EPHB2 | 6670405 | 23848017 | 1 | 22934429 | T | C | CCGTATAATCATCTNAATTGTCTGTAACC |
| 25 | EPHB2 | 908503 | 23168361 | 1 | 22940620 | A | G | GGGGTCATTGTTCTNATCTACCATGCACG |
| 26 | EPHB2 | 10917325 | 24630944 | 1 | 22954584 | C | T | CCTTTCATCTTGACNTCAGTGGCTTGATG |
| 27 | NTNG1 | 11185063 | 23172592 | 1 | 107390776 | C | T | ACTACGTGTTCTGTNTGATTCTCAAAGAA |
| 28 | NTNG1 | 11185064 | 24254044 | 1 | 107401214 | G | T | AAGATTTCAAAATGNGCTTTACCCAGTAT |
| 29 | NTNG1 | 4915020 | 24254267 | 1 | 107417200 | C | T | TCACTTGGAAGTTGNATAAGCATCACCAA |
| 30 | NTNG1 | 7522251 | 23173004 | 1 | 107425312 | G | T | GGATTAATTGGATANATTATTTGGTATTT |
| 31 | NTNG1 | 4556390 | 23173101 | 1 | 107431339 | G | A | GATAAACGGAATGTNATTTTTTACAATCT |
| 32 | NTNG1 | 4311917 | 23173173 | 1 | 107437785 | C | A | AGAGCATTTAATTCNGTGCTGGAAGAGGT |
| 33 | NTNG1 | • | 46535862 | 1 | 107452170 | C | T | GACAATATTTAGACNCATTGAGTGTTAGA |
| 34 | NTNG1 | 4430358 | 23173416 | 1 | 107475426 | T | C | TATCATTAAGACTCNATGCATTTCCTAGT |
| 35 | NTNG1 | 10159449 | 23173534 | 1 | 107488346 | A | G | GATAGCTTTCAAATNCAATGATTAGAGAG |
| 36 | NTNG1 | 12731259 | 24254770 | 1 | 107502674 | A | G | AACTAUGAGTGGTCNTTCAAAATGATCCC |
| 37 | NTNG1 | 11185076 | 23850970 | 1 | 107508742 | T | A | GTGGGGAAAATATCNTTTAGGATTCATCA |
| 38 | NTNG1 | 4579786 | 23173971 | 1 | 107528897 | A | C | CCATTCTGGGAAAANAAGTAACATGATGA |
| 39 | NTNG1 | 3123375 | 24255006 | 1 | 107535321 | T | A | TATAACTCTCCTTTNTGAATGAGAAGCCT |
| 40 | NTNG1 | 6694986 | 23851153 | 1 | 107541720 | T | C | GGTTTTCTGAGGCANTTTGAAGGCTCATT |
| 41 | NTNG1 | 11185082 | 23851200 | 1 | 107554750 | A | G | TCCAGAACTCTTCTNTAGCAAAACAAGAA |
| 42 | NTNG1 | 11185084 | 24255157 | 1 | 107560725 | G | A | GTGAGAAGTAGAAANCACCACTATGATAT |
| 43 | NTNG1 | 6678045 | 24255209 | 1 | 107567192 | A | G | TGTAGCTAAGTGGTNGAGCTGGAATTCAA |
| 44 | NTNG1 | 7542677 | 24255229 | 1 | 107574095 | C | T | CAATCTCTGAGAAANGAATAGTAGGCTCT |
| 45 | NTNG1 | 7555385 | 23174634 | 1 | 107601690 | G | T | GAATAAAGAACAAGNCAAAGCAAGCAGGA |
| 46 | NTNG1 | 4542239 | 24255397 | 1 | 107622225 | G | A | TTGCATCTTGGCAANCCTGGAATGTCACA |
| 47 | NTNG1 | 6662404 | 23851449 | 1 | 107631565 | C | T | AACTATCTGAATAANGCCTCTTCTCAAAT |
| 48 | NTNG1 | 11185105 | 24255511 | 1 | 107645587 | G | A | TTATTATGCAACCANCCTTGCCTTATGTC |
| 49 | NTNG1 | • | 46535843 | 1 | 107654602 | C | A | CTTCAAAGATTCCCNTTCTATTAGTCTTT |
| 50 | NTNG1 | 2218404 | 23851652 | 1 | 107664647 | T | G | TAGACGACACTGCCNGTTCCAGAGGGCAC |
| 51 | NTNG1 | 4915043 | 24256010 | 1 | 107678161 | C | T | CTGAAGGCCTGGATNGCATTCCCTTTGTC |
| 52 | NTNG1 | 12089521 | 24256045 | 1 | 107684404 | T | C | GATACCCCACCTAGNTACCACTCACACTT |
| 53 | NTNG1 | 10748506 | 24256136 | 1 | 107690524 | G | T | ATAGAAGCCTAAAGNATATCCATTTCTGG |
| 54 | NTNG1 | 17019069 | 23175804 | 1 | 107697674 | G | A | CAGACTCTGGAGAANTTCCAATAGAAGCT |
| 55 | NTNG1 | 12117762 | 23852066 | 1 | 107707884 | T | C | GCAGCACAGTAAACNTCAAGTTTGTTAAT |
| 56 | NTNG1 | 4914945 | 24256414 | 1 | 107714402 | T | A | AAACACCTTCAGAANAGAGCACTTTAACC |
| 57 | NTNG1 | • | 46535839 | 1 | 107723900 | A | G | TAATACAAGTCACCNTTCTCTTCCTGTTC |
| 58 | NTNG1 | 96501 | 23176261 | 1 | 107730889 | C | T | TTTCTTCTCAGCATNCTTTATGGTCTTTC |
| 59 | NTNG1 | 597332 | 24256714 | 1 | 107736910 | T | C | GGAGTATCACAAGANGCAACCTGGGGAAC |
| 60 | NTNG1 | 503828 | 24256839 | 1 | 107746271 | G | A | CCCTGGAAGTCATANCATTGCAGAGGCTG |
| 61 | GNAI3_GPR61 | 552101 | 23185781 | 1 | 109794515 | G | T | CACTCTGCTTTGCCNGCTGCCTCTCACCG |
| 62 | GNAI3_GPR61 | 3737182 | 24263446 | 1 | 109803313 | C | T | AGCAATAGACGGTGNCTCAGCCTGCCGAG |
| 63 | GNAI3 | 12023950 | 24263473 | 1 | 109811900 | T | C | ACCATGCCTCGCCANTGGTGAAACTGTTT |
| 64 | GNAI3 | 6692804 | 23185950 | 1 | 109820784 | G | A | AAAGGGAATAATCTNTGCCAGCCACCAGG |
| 65 | GNAI3 | 11803546 | 24263579 | 1 | 109831527 | G | A | AGAGGGTTTATTGTNGGGAAGCAAGAGCA |
| 66 | GNAI3 | 12033376 | 23202109 | 1 | 109842683 | A | T | ATTCTTTCTTTCCTNACCAGCTTTCTCTG |
| 67 | GNAI3_GNAT | 559479 | 24274941 | 1 | 109850578 | T | A | TTAGTACTTGTGGGNTCTTTCCTTTTCAT |
| | 2 | | | | | | | |
| 68 | D1S155E_NRA | 10489525 | 23191563 | 1 | 114975727 | G | A | TATCTTCTTCCCAANTTCACAATATGAGC |
| | S | | | | | | | |
| 69 | SEMA6C | 4357530 | 24278041 | 1 | 147916226 | G | A | CTTGGTTTAAGTAANGCACCCCATACGGG |
| 70 | SEMA6C | 12088941 | 23206664 | 1 | 147931753 | T | C | CGACGAAAGCCCAANTGAACGAGGTGCCC |
| 71 | DPM3_EFNA1_ | • | 46535665 | 1 | 151928333 | T | C | GGCCTAGGTGGGAGNGTTGCTTGAAGAAA |
| | LOC55974 | | | | | | | |
| 72 | SEMA4A | 11805565 | 24638319 | 1 | 152933561 | C | G | AAGCTCAGAGGTAGNATAGAGTCGGGGCG |
| 73 | SEMA4A | 6701295 | 24638342 | 1 | 152961238 | T | C | GATCTGTTCTCTAANCATCTGACCTCCAG |
| 74 | FNBP2 | 17045185 | 23702866 | 1 | 202974625 | G | T | CCTGCAGACTCTGANATGCCTTTACCTTT |
| 75 | FNBP2 | 9430095 | 24642037 | 1 | 202982299 | G | C | AGATTCAAGGTGTCNTAGGATTCCAAAUG |
| 76 | FNBP2 | 2297539 | 23702917 | 1 | 202991934 | G | A | CAGGCAATTCCTCTNGTGGTGGAAAGCTG |
| 77 | FNBP2 | 2483064 | 23702928 | 1 | 203003171 | G | A | CTCGTCCAAGCAUCNTGTCCTTGTGTGGA |
| 78 | FNBP2 | 2026081 | 23702954 | 1 | 203014267 | T | C | AATUCTTAAAAATANAGTATGGCAAGYTT |
| 79 | LOC439926_P | • | 46535089 | 1 | 204579018 | T | G | GCTCTTCTAAATGGNCTGGGATAATTCAG |
| | LXNA2 | | | | | | | |
| 80 | LOC439926_P | 6659522 | 23669188 | 1 | 204587495 | T | A | CTCTTTGTTTGGAANCTACACTCAGGAGA |
| | LXNA2 | | | | | | | |
| 81 | PLXNA2 | 4844633 | 24142021 | 1 | 204597780 | G | T | CCATTGACTTGATGNTATAAAGCTCCAAG |
| 82 | PLXNA2 | 2281913 | 24142025 | 1 | 204604512 | T | A | CCTGTGTCTTTCTTNAAGCCATCTTAAAT |
| 83 | PLXNA2 | 7514550 | 23669195 | 1 | 204610043 | G | T | GTAGGCCCAGGTGTNCTGAGGATGAGCTG |
| 84 | PLXNA2 | 11118979 | 24142028 | 1 | 204617623 | T | G | TAACATGCAATAACNGTCTTATTACGTTT |
| 85 | PLXNA2 | 12057548 | 24142031 | 1 | 204624559 | G | A | ACTATAGCTCATCTNTCCCTGCACTCTAA |
| 86 | PLXNA2 | 6656034 | 24142034 | 1 | 204627571 | A | G | CATCCTTTCACCCANAATAAGTTGGAAG |
| 87 | PLXNA2 | 2478819 | 23669224 | 1 | 204662509 | C | T | GAAAGCTTGTTTGGNTGTTTCTCCCAACA |
| 88 | PLXNA2 | 4844406 | 24617588 | 1 | 204670334 | G | A | CTGGAGAAACGCCTNAGGTGAAAGCAGAA |
| 89 | PLXNA2 | 4844409 | 23669233 | 1 | 204677703 | C | T | TTATAGTCCAAACANCTCAGTGTGACATT |
| 90 | PLXNA2 | 12568876 | 23669237 | 1 | 204685422 | C | G | AAAAGAATGACTCTNCTCTAATGGTTATC |
| 91 | PLXNA2 | 895241 | 23669250 | 1 | 204691028 | C | T | TAGCATCTTTTTTCNTGATGATGTTACTT |
| 92 | PLXNA2 | 10863700 | 23669255 | 1 | 204695814 | C | G | TGAGGTTTTTGGCANCACTTCTCCCAGCC |
| 93 | PLXNA2 | 1875431 | 23669259 | 1 | 204709513 | C | T | GGCAAACATAGCTGNCCTTGCTGCTGAGC |
| 94 | PLXNA2 | 632165 | 24142052 | 1 | 204717618 | T | G | GGAGGCTGCCTGGGNCTGGCTGACAACCA |
| 95 | PLXNA2 | 2016717 | 24617617 | 1 | 204736606 | C | A | CAAGAGAAATTCCCNGAGGAAATGAGCTG |
| 96 | PLXNA2 | 1892286 | 24617618 | 1 | 204747506 | G | A | ATTGTCTTAAGAGANTAATAAGCTTTGTG |
| 97 | PLXNA2 | 11590686 | 24142059 | 1 | 204756596 | C | T | TTTCCCAAGGGCACNTTTTCTGGAGCAAA |
| 98 | PLXNA2 | 12142104 | 24617625 | 1 | 204769172 | T | C | AGTGTATAAGCAGCNATGTGGACACAGCA |
| 99 | PLXNA2 | • | 46535086 | 1 | 204799243 | C | T | CCTGGAGTCCTTGCNGTAGGATATTGACT |
| 100 | PLXNA2 | 17012229 | 24142066 | 1 | 204812093 | A | G | GTGGGGGCCCATCANTGTGGGAGTTTGAA |
| 101 | C2orf22_ROC | 6753921 | 24645321 | 2 | 11277353 | G | A | AAATCAAAACTCCTNAGCACAGTGTACAT |
| | K2 | | | | | | | |
| 102 | ROCK2 | 12470004 | 24645338 | 2 | 11286630 | T | C | AATCAGTAACATTGNAATTCAACTAAGGA |
| 103 | ROCK2 | 17366657 | 24645352 | 2 | 11295112 | T | C | CTCCTATCCTGTCANTGTTTTTGAAATTT |
| 104 | ROCK2 | 7598032 | 24645368 | 2 | 11303798 | T | C | TATCTAATCAACTANTAAGTGCTGCCAAA |
| 105 | ROCK2 | 9808232 | 24645383 | 2 | 11309718 | G | T | ATTGTATGGAATCANTTTCTCTACAAGAT |
| 106 | ROCK2 | 10167277 | 24163456 | 2 | 11316598 | T | A | GTCATACTATGACANGAAACAAAGCATAA |
| 107 | ROCK2 | 13393192 | 24645404 | 2 | 11319264 | T | C | CTTTGTGTGGATTANTACAAAGGACAAAT |
| 108 | ROCK2 | 6721445 | 23707505 | 2 | 11346706 | C | G | AAGTCAGTGAACTTNAACACAAATCAATC |
| 109 | ROCK2 | 4669700 | 24163482 | 2 | 11352136 | T | C | CCTCAGCATCTCTGNAGATTTCCCTTTAT |
| 110 | ROCK2 | 4668720 | 23707533 | 2 | 11356412 | T | C | AGCAATTTAGACCTNTGTGAATGAGAATG |
| 111 | ROCK2 | • | 46539209 | 2 | 11388527 | T | A | CTCTCTGAGTACCTNTATGAACAAAATTT |
| 112 | ROCK2 | 6713273 | 23707554 | 2 | 11398932 | C | T | AAGAAAGAAAACCANAGGCAAAATTGCAA |
| 113 | ROCK2 | 12992581 | 23707593 | 2 | 11406971 | T | C | GGTGGAAGAGGAGGNGATGGATAGGCCTT |
| 114 | ROCK2 | • | 465392082 | 2 | 11417111 | T | G | ACATTCTATAAGATNATGGTTTTAGCAGC |
| 115 | DPYSL5 | • | 46539093 | 2 | 26976228 | T | C | AATGGAATAATGTTNAGTAACAGGTAGTG |
| 116 | DPYSL5 | • | 46539092 | 2 | 26994920 | T | C | CTGCTTTGATGTCCNGTGACAGACACCCT |
| 117 | DPYSL5 | 486582 | 23304562 | 2 | 27015782 | T | C | CCCTGAAATCCAGANGCGATCAGCCTAGT |
| 118 | DPYSL5 | 17005660 | 23694134 | 2 | 27038346 | C | G | GTTAAGTTCCCTAANCCGGTGTCCTGACC |
| 119 | DPYSL5 | 12615423 | 24635831 | 2 | 27070274 | G | T | TTTTGGGTATTAAANTTCCATCCAGTTTA |
| 120 | LOC56902_PP | 875 | 24265772 | 2 | 68319577 | A | G | TTATTGAGAGAATTNCAGTTCAATAACAC |
| | P3R1 | | | | | | | |
| 121 | PPP3R1 | 6546359 | 24265838 | 2 | 68329729 | C | G | TTAATTTGTACTTGNATTTTTAAGATCAT |
| 122 | PPP3R1 | 7577500 | 24265874 | 2 | 68342947 | C | T | CCAAAGTTATCATGNCAAGTTAGAACTAA |
| 123 | PPP3R1 | 12713636 | 23860279 | 2 | 68347082 | C | G | ATAATAGTTCTCAANTCCAGAAGCCCACT |
| 124 | PPP3R1 | 10208241 | 23860303 | 2 | 68358587 | C | T | AATATGAAAAAAGTNCTAAAGCTAACATG |
| 125 | PPP3R1 | • | 46538705 | 2 | 68375263 | G | A | AACAGCTATGCTCCNCTTGAAAGAGCTTC |
| 126 | PPP3R1 | 7560138 | 24266081 | 2 | 68386417 | A | G | AGTTTCTCGAGGACNGGAACAGTATTCCC |
| 127 | LOC130951_S | 10175000 | 23220107 | 2 | 74787538 | C | G | TATAGCTGCCTTCANAAAGTTCTTTTAAG |
| | EMA4F | | | | | | | |
| 128 | SEMA4F | 363612 | 23220518 | 2 | 74822728 | T | C | GATGAACCTACCTANAGATAGAGCAGAGT |
| 129 | MGC41816_SE | 6576989 | 23211899 | 2 | 96946973 | T | C | CCTCACACAAGCAANGATCTCATCTCCAC |
| | MA4C | | | | | | | |
| 130 | NCK2 | 6543342 | 23902800 | 2 | 105920774 | T | C | AATGGTGACTGTTANCCGTCTACTTTGGG |
| 131 | NCK2 | 7580718 | 23902867 | 2 | 105929845 | C | A | TCCTGTTTCTAAACNAAATACTAAAGCTT |
| 132 | NCK2 | 3769500 | 23902927 | 2 | 105939942 | G | A | CATCCAAATAACACNAGTACCTTCAACTA |
| 133 | NCK2 | • | 46537105 | 2 | 105947913 | A | G | TCCCGGAAGTGGACNTTAGCAGCGCCTGT |
| 134 | NCK2 | 3739131 | 23876083 | 2 | 105957177 | G | C | GTTTGCTACTCCGTNATTTACTTGCATCT |
| 135 | NCK2 | 10469862 | 24286175 | 2 | 105968425 | C | T | TCGTGCATTCAACTNGTTCCCGCTCATGG |
| 136 | CXCR4 | 2734871 | 23708651 | 2 | 136703605 | G | A | AAGGCTTTGCAAGANAAAACATCCACTTT |
| 137 | CXCR4 | 9973445 | 24164129 | 2 | 136712348 | C | G | ATCTACTAAGATGTNAGACTTTTTTCACA |
| 138 | EPHA4 | 7422206 | 24343062 | 2 | 222104874 | T | A | TATTTTATTACATCNTGCTCAGTCTGTAC |
| 139 | EPHA4 | 13386128 | 23301862 | 2 | 222110870 | G | C | GAATATATAGGTTCNTAGAAGTTGGTATC |
| 140 | EPHA4 | 2303901 | 24343274 | 2 | 222116407 | A | G | ACAGAAGAAGTCACNGGGACTGACAATCT |
| 141 | EPHA4 | 10211091 | 23302038 | 2 | 222119773 | G | A | AACTTAAAATTGCCNTGCTTAAAGACCTG |
| 142 | EPHA4 | 3770192 | 24343418 | 2 | 222133561 | G | A | AAATTTGTACTAGANTTCTTTATAACACA |
| 143 | EPHA4 | 2052942 | 23302449 | 2 | 222142080 | T | G | TTCACCGCGATCCCNAAATGCTTACATGT |
| 144 | EPHA4 | 13026588 | 24343802 | 2 | 222155674 | C | T | GATGCTATTCTTAGNCTTCACGTTAATTG |
| 145 | EPHA4 | 975194 | 23302962 | 2 | 222163131 | G | A | AGGCCTACCAATTTNTCAGAATAGAGAAA |
| 146 | EPHA4 | 3770148 | 23922446 | 2 | 222168892 | C | A | GGAGTGACTCATCTNATTAGAGAATTTGG |
| 147 | EPHA4 | 16862777 | 23922589 | 2 | 222180978 | G | C | AAGAGTCAACAGTTNGGATGCATfACGTT |
| 148 | EPHA4 | 7598307 | 24344336 | 2 | 222194490 | C | T | AAGATTAGTTCACANGTATGATGCAAACT |
| 149 | EPHA4 | 7558703 | 23922719 | 2 | 222199928 | T | C | AITTAGGACAGAGANGTTTCACAGACAAC |
| 150 | EPHA4 | 2710507 | 23922771 | 2 | 222206305 | G | A | TTCCTCAAGGCTGTNTTAAGGTTACATTT |
| 151 | EPHA4 | 2680848 | 24333335 | 2 | 222221395 | T | G | ATTAGTATTGAAACNGCAGAATTAGGTTA |
| 152 | EPHA4 | 2710493 | 24333548 | 2 | 222233842 | T | A | GGCTTTCTATGCTGNACTTAGCAGCTACG |
| 153 | EPHA4 | 2710491 | 24333562 | 2 | 222235873 | G | A | TTTTCTGAAACTAANGATGGAATAACCAT |
| 154 | EPHA4 | 10498118 | 23914596 | 2 | 222248894 | G | A | AAAATCCCTGAGATNATTTCTATGCGATG |
| 155 | EPHA4_LOC4 | 7340471 | 23288344 | 2 | 222257825 | T | C | GCAATCTGTTTTATNTCAGGTGGATTACA |
| | 02120_LOC440 | | | | | | | |
| | 933 | | | | | | | |
| 156 | NGEF | • | 46537033 | 2 | 233606587 | G | A | AGCTAAGAAGTAGCNGAGCTGAGAAACCT |
| 157 | NGEF | • | 46536997 | 2 | 233635802 | A | G | CAAGGGTGCCAGCCNCCACCAGAAGCCAG |
| 158 | NGEF | 4973583 | 24276333 | 2 | 233644264 | G | A | TTCTTCCTATTGGCNTGAGGATAACAGAG |
| 159 | NGEF | 7583124 | 24280058 | 2 | 233661159 | C | T | CAATAAAACCATAANGATTGGTGTTATTT |
| 160 | NGEF | • | 46537386 | 2 | 233671260 | G | A | CATCTCTCGCTATCNTCTTCTGCCTCTCA |
| 161 | SRGAP2 | 780907 | 23238606 | 3 | 8990504 | A | G | TTGCATCGGCCCTCNGATAAAGAGAAGCA |
| 162 | SRGAP2 | 341791 | 23886846 | 3 | 8999017 | A | G | CAGTACGGCTTTCANTAATGGTGATGAAC |
| 163 | SRGAP2 | • | 46539676 | 3 | 9017691 | T | C | AAATAAGATGTATANGGTAAACCTTAGAG |
| 164 | SRGAP2 | 399747 | 23886920 | 3 | 9027506 | G | T | ACATATCCCAGGAGNTAAGATGAAGCTAA |
| 165 | SRGAP2 | 6443217 | 24299606 | 3 | 9037146 | A | G | AATTCAAAAACTGTNCCATATTAAGCTGT |
| 166 | SRGAP2 | 7617156 | 24299764 | 3 | 9047878 | T | C | TTTGTGTTTAAAATNGCCCAAGCTCCACT |
| 167 | SRGAP2 | 3933080 | 24299928 | 3 | 9060419 | G | T | TCAGTAATTTTCTANAAAGTCAGATGGCC |
| 168 | SRGAP2 | 13091604 | 24300018 | 3 | 9069531 | T | C | TACCCATTGGGAATNCACTTTCTGCATTT |
| 169 | SRGAP2 | 1162395 | 23887339 | 3 | 9086221 | T | C | GGACTTCGTAGGGANGTGTCAGTTAAGAC |
| 170 | SRGAP2 | 1162393 | 24300192 | 3 | 9099624 | G | A | CAGCAGAAACCTCGNAGGCAATTCCAGAG |
| 171 | SRGAP2 | 1162394 | 24300199 | 3 | 9100744 | G | C | ATCTTCCTGGGCTTNTCAACTGGATACCT |
| 172 | SRGAP2 | 2664073 | 24135002 | 3 | 9124099 | C | T | GTTCTCCTCCAGTGNGTTCTGGGTACCAG |
| 173 | SRGAP2 | 13078449 | 23656283 | 3 | 9137915 | T | C | AGCACCTGACCCACNGCAATGGGAATAAA |
| 174 | SRGAP2 | 2077115 | 24135008 | 3 | 9140840 | A | G | TCTCAGAGCTTGGGNTATGAGAAATCAGG |
| 175 | SRGAP2 | • | 46539673 | 3 | 9154090 | A | G | CAGTGCTGGGTGAANTGACTGGCATCTCC |
| 176 | SRGAP2 | 2257056 | 24608153 | 3 | 9163615 | T | G | TGCTGTGATGCAATNTAGGCTATGAGTGG |
| 177 | SRGAP2 | 6762693 | 23191239 | 3 | 9170331 | T | C | ATGCAGTGGTAACANAGGAACATTTAACA |
| 178 | SRGAP2 | 2668349 | 24135010 | 3 | 9177419 | C | T | GTGAAGAATTGATANACGTAAAGGGTTAA |
| 179 | SRGAP2 | 396323 | 24608158 | 3 | 9190296 | T | C | CAGAAATCTAAGCANCAATCCCTCCAACG |
| 180 | SRGAP2 | 422810 | 23656289 | 3 | 9193797 | A | G | ACTAAAACTTCAGGNAAAATCGTGTGTTC |
| 181 | SRGAP2 | 1154389 | 24608160 | 3 | 9207362 | C | T | AGTCGGTCAGCTATNTGGGGCCTGAATTA |
| 182 | SRGAP2 | 17050161 | 23656301 | 3 | 9214315 | T | C | TCCAGAAGACCATGNAACTTGTCTATAGT |
| 183 | SRGAP2 | • | 46541670 | 3 | 9220822 | A | G | AACCATCAAACACCNGGAACCCATCTTGG |
| 184 | SRGAP2 | • | 46541667 | 3 | 9231186 | C | T | CAGTGCTTCCCAGTNACTGAACAAAAACT |
| 185 | SRGAP2 | 2700323 | 23887490 | 3 | 9248236 | A | T | TATTTTTATAGGCCNACACCGAACTGTGT |
| 186 | SRGAP2 | 2137215 | 23239891 | 3 | 9253830 | G | A | ATGCACCCCAAAACNTAATATCTATGTGT |
| 187 | SRGAP2 | 17744749 | 24300372 | 3 | 9266441 | A | G | GGAGAGCAGAACTCNCTGCAAGCAAAAAG |
| 188 | SRGAP2 | 6787581 | 23887605 | 3 | 9275554 | G | C | TCCCTCCTATTTGCNATCAACCCTGGAAC |
| 189 | FBXW12_PLX | 1563736 | 23867468 | 3 | 48411798 | G | A | AATTGGAGAATGAANAAAATCCAGAAAAT |
| | NB1 | | | | | | | |
| 190 | FBXW12_PLX | • | 46539719 | 3 | 48418820 | C | T | TGTTCTGTGGCGGCNGAGCCATGACTCAG |
| | NB1 | | | | | | | |
| 191 | PLXNB1 | 10470686 | 23867531 | 3 | 48424960 | C | T | CGGACGGCTCCAACNACTCAGTCTCCTGT |
| 192 | GPX1_RHOA | 17080528 | 23203153 | 3 | 49364846 | C | T | CAGCCAGGATCAGGNATACTGGGAGTTCA |
| 193 | GPX1_RHOA | 974495 | 24275210 | 3 | 49375486 | T | C | CCAGGGTCCACATGNTCTTTTTCTAACTT |
| 194 | RHOA | 7621003 | 24275246 | 3 | 49386408 | T | C | TGCCAGAGGGCTTTNAGGACAGAAATGGG |
| 195 | RHOA | • | 46539701 | 3 | 49407325 | C | T | GGAAAGAGCGGTAANGATGGTTCAGGAAT |
| 196 | AMT_RHOA_ | • | 46541286 | 3 | 49424642 | G | A | CTGGTTGCTATTTTNTACTCCCGGAGTCA |
| | TCTA | | | | | | | |
| 197 | AMT_NICN1_ | • | 38339034 | 3 | 49429281 | G | A | TAATGCATACCAGGNGCAAAGATCAGCCC |
| | RHOA_TCTA | | | | | | | |
| 198 | SEMA3F | • | 46541293 | 3 | 50164061 | C | T | GCCAATGACTTCCCNGTTGTAACCCTCAC |
| 199 | SEMA3F | • | 46541291 | 3 | 50171422 | C | A | CTTTGATTTTAGTANAAGTGGACTCTGCC |
| 200 | GNAT1_SEM | • | 46539367 | 3 | 50194995 | C | G | GAGTTGGGGGTGTTNTTGCACCTGGCTGG |
| | A3F | | | | | | | |
| 201 | GNA12 | 10510755 | 23868602 | 3 | 50256172 | C | T | TTTATGCACTTAAANAGGGAACAGAGATG |
| 202 | GNA12 | 9852677 | 23204030 | 3 | 50266621 | G | A | TCCCTGTGGTATCTNTCATGTACTTGGAC |
| 203 | FLJ38608_HY | 12494414 | 23868652 | 3 | 50298176 | C | T | ACCTTCATATTCAANGGTGGATGTCCTGG |
| | AL3_IFRD2_S | | | | | | | |
| | EMA3B | | | | | | | |
| 204 | LOC56920_NI | 10865971 | 23281082 | 3 | 52456446 | A | G | TCTTGATGGAGGAANTCAAGGGAGGCTTC |
| | SCH_TNNC1 | | | | | | | |
| 205 | ROBO2 | • | 46541014 | 3 | 77601166 | T | G | ATCCTGAGGATCGGNCTCCCCCTGGGGTC |
| 206 | ROBO2 | • | 46541013 | 3 | 77608306 | A | C | TGTTGGAATGTCCANGAACAGAAATAGTA |
| 207 | ROBO2 | 17015294 | 23890743 | 3 | 77618836 | G | A | TATTTTCACAGTACNATGTTCAGATTGAG |
| 208 | ROBO2 | 6794832 | 23245250 | 3 | 77626835 | G | C | GTATTAGTAGAAGANGTTTGTAAATGTGA |
| 209 | ROBO2 | 12489432 | 23890807 | 3 | 77630572 | G | A | GCTTCTGGGACACANAGATGCTGAACAGG |
| 210 | ROBO2 | 9309770 | 14908996 | 3 | 77647000 | G | C | TCTGATCACAATTANAGGCCTATCCTTTC |
| 211 | ROBO2 | 6767931 | 23890912 | 3 | 77664762 | T | C | GAAGACACAGGTTCNTTATCTCTCTTTAA |
| 212 | ROBO2 | 17015331 | 23890933 | 3 | 77671228 | T | C | GAATCGGTTAGATTNGTATTCAGGTAACA |
| 213 | ROBO2 | 6783764 | 24265866 | 3 | 77683424 | A | G | CTCTGCGTAATCTGNTTTTTTCCCAGCAG |
| 214 | ROBO2 | 1109413 | 24304656 | 3 | 77702723 | A | C | ATGACCAAAGGCACNAGACATAACTACTG |
| 215 | ROBO2 | 1666132 | 23891087 | 3 | 77731880 | C | T | AGAGGAAGCATTTCNATAACTCATAGGTT |
| 216 | ROBO2 | 1721175 | 23891093 | 3 | 77733384 | C | T | ATGAAACTGTAATTNTCTAGTTCGGAGTT |
| 217 | ROBO2 | 1163748 | 24329692 | 3 | 77780633 | G | A | CACAGCAGTTTCTCNTTTCATAGCCGATT |
| 218 | ROBO1 | 13070644 | 23915040 | 3 | 78726324 | C | T | GAGTAAGTCACTGTNGAACCTTTATCTGA |
| 219 | ROBO1 | 12496962 | 24334119 | 3 | 78735521 | C | T | AAATCAGGATACCTNTTTCACTATAAACA |
| 220 | ROBO1 | 1027833 | 24334222 | 3 | 78746783 | C | T | TAAACAAACTCTTCNAGCAAATAACAGGC |
| 221 | ROBO1 | 17016412 | 23915156 | 3 | 78753263 | A | C | GAAAGGATGCCAGCNTTGTTGTGTTTGGA |
| 222 | ROBO1 | 3821596 | 24334352 | 3 | 78759728 | A | G | CCAATATGTTCTCTNAAAGACTCAAAGAT |
| 223 | ROBO1 | 3821597 | 16335307 | 3 | 78771951 | G | A | CCATAACTGTACACNGGAAGAGTTATGCA |
| 224 | ROBO1 | • | 46540995 | 3 | 78784493 | C | T | AATAATAATCATGGNGAGCAACAGATAAA |
| 225 | ROBO1 | • | 46540994 | 3 | 78794040 | T | C | AAACTGTTCATTCANAGCAACTCTCAGTA |
| 226 | ROBO1 | 6788434 | 23289716 | 3 | 78816377 | G | A | CAGTTTACGTAATCNGGAAAATAAGAGTG |
| 227 | ROBO1 | 17016456 | 23915408 | 3 | 78823838 | T | C | CTTTCTAGTCTATTNAGGCATTTTTGTTG |
| 228 | ROBO1 | 17016466 | 23289872 | 3 | 78837168 | A | G | ACAACTGTATTTGCNTAAGCACCCCATAA |
| 229 | ROBO1 | 6785152 | 24334779 | 3 | 78845821 | C | T | AATTGTACAGGCCCNTGAAAGTGGATGAT |
| 230 | ROBO1 | 2164549 | 24334793 | 3 | 78851827 | T | A | AATTTTTGTCTTTANCATAACCCTGCTGT |
| 231 | ROBO1 | • | 46540992 | 3 | 78865774 | C | T | GCTGGGGTGGCAACNTAATCCAAAATTCC |
| 232 | ROBO1 | 17016495 | 23290020 | 3 | 78875814 | C | T | TACTTGTATGCCTANCACGATGTAATTTT |
| 233 | ROBO1 | 416025 | 23915662 | 3 | 78886724 | C | T | GTAAAACTTATCCTNGGGAAGTGCATTGC |
| 234 | ROBO1 | • | 46540991 | 3 | 78897021 | G | A | AGTTTCTATATGCANATCATGACTGAATA |
| 235 | ROBO1 | • | 46540990 | 3 | 78905774 | C | G | TGGATATCTTGGCTNTGTCACTGTTTCCC |
| 236 | ROBO1 | • | 46540989 | 3 | 78914822 | T | C | GTGCTGGGCAATCANATAGGCACTGTGAA |
| 237 | ROBO1 | 7650002 | 23290459 | 3 | 78926964 | G | A | CCTGTAAAAAGATANCATTCCCTTCCAAT |
| 238 | ROBO1 | 6548600 | 24335151 | 3 | 78932798 | G | A | AATAGGATAAATACNGATAATCTTAATAA |
| 239 | ROBO1 | 11127635 | 23915896 | 3 | 78940492 | A | G | ATGGCTTAAATTATNCTGTTAAAAGCAAT |
| 240 | ROBO1 | 7629522 | 24335217 | 3 | 78945848 | C | T | TACCATGCTATTCANTTCGGTATATGCCA |
| 241 | ROBO1 | 3856695 | 23915971 | 3 | 78956626 | G | A | CAAATAACATAATGNTTGCATGCACATAG |
| 242 | ROBO1 | 7617433 | 24335287 | 3 | 78966169 | G | A | AGTTATTTTGGTGGNAACGGCTTAGGTGT |
| 243 | ROBO1 | 17016554 | 23916051 | 3 | 78973288 | C | T | CAAGTACAAGGTCTNTGCAGTGTGTTGGC |
| 244 | ROBO1 | 9835958 | 23916086 | 3 | 78979288 | T | C | TAGAGTACCTGAAGNTGTACCTCAAAATA |
| 245 | ROBO1 | 9309820 | 23916106 | 3 | 78983266 | T | C | GTTACCAGTCCTTANGGCATTTGGGTAAG |
| 246 | ROBO1 | • | 46541007 | 3 | 79003534 | A | C | CTGTCCPTTCATAGNCCTCAGTACTAGAA |
| 247 | ROBO1 | • | 46541005 | 3 | 79014481 | A | G | TACTCAATGTCAGTNTATCCATCCTCAAT |
| 248 | ROBO1 | 329820 | 24335552 | 3 | 79030645 | C | T | CATGGAAGTCCATANATAAATTTTGTGTA |
| 249 | ROBO1 | 162264 | 23291025 | 3 | 79037961 | C | T | ATTTCAGAATCCTANGATTTTTCCTGCCA |
| 250 | ROBO1 | 328045 | 24335615 | 3 | 79045254 | T | G | CTTAGTTTTCAATTNTTAAAATGTCAAGC |
| 251 | ROBO1 | 189022 | 24335729 | 3 | 79065987 | T | A | GTGTCTAACCATAANGGAAGTAAAGAGAA |
| 252 | ROBO1 | 9822620 | 23291302 | 3 | 79075910 | G | T | ATCCACCATCTTCTNTATTACAACAACAT |
| 253 | ROBO1 | 7617538 | 24335815 | 3 | 79084475 | A | T | CTCTATTAAGTTACNCACATTTCCAAATA |
| 254 | ROBO1 | 9818387 | 23291359 | 3 | 79090086 | G | A | TAATCTCTGGGTACNAAGACTAGTAACTG |
| 255 | ROBO1 | 17313732 | 24335862 | 3 | 79096568 | G | A | GAAATGAAAATTACNAAGAAAGAACTCAT |
| 256 | ROBO1 | 4680919 | 23916509 | 3 | 79108259 | T | C | GTGCCGGTGAGTCTNTGCGCTTATCAGGA |
| 257 | ROBO1 | 17016680 | 23916566 | 3 | 79125696 | A | G | AAATCTGTTAAGCCNAAATGGTTTTTTTT |
| 258 | ROBO1 | 11708079 | 23916631 | 3 | 79138475 | A | G | GAAAGTATGTCTACNTTGAGGCTGTATTT |
| 259 | ROBO1 | 6790493 | 23916668 | 3 | 79143910 | T | G | AACTTGATACTTTCNGAAATCTAACCAAG |
| 260 | ROBO1 | 9866364 | 23291933 | 3 | 79240044 | C | T | TACATAGAAAGTACNCCAAGATACATCGA |
| 261 | ROBO1 | 17315187 | 24336280 | 3 | 79252565 | T | C | AGCATCTAGAATGTNAAAAAAATGCAACT |
| 262 | ROBO1 | 6786179 | 24336347 | 3 | 79259720 | A | G | TCTTTGCATTAATCNATGAATCTTACCAT |
| 263 | ROBO1 | 9867774 | 24336366 | 3 | 79270875 | G | A | AATTGTCAAGTAGANTTTGGTTTAAGTTA |
| 264 | ROBO1 | 7638301 | 24336500 | 3 | 79279060 | C | G | AGCATGGTTTAAAGNCTGATTTTACTCTA |
| 265 | ROBO1 | 2311349 | 24336527 | 3 | 79280684 | C | T | TTAGCTGTATTTTGNTGATGTGATCAATA |
| 266 | ROBO1 | 966144 | 23292354 | 3 | 79317376 | G | T | TTGATTTCCTATTANGTATTCATCTTAGA |
| 267 | ROBO1 | 9845808 | 23292472 | 3 | 79331518 | C | T | ACATTTCCATGAGANACATATTTTTAGAA |
| 268 | ROBO1 | 17044180 | 23292507 | 3 | 79344330 | A | G | CGACTGTCTTTTTTNGTGAATAACAGTGA |
| 269 | ROBO1 | 11920171 | 23292585 | 3 | 79354884 | G | T | AGCATGAGTTCTCCNGTAGGTACTAATTG |
| 270 | ROBO1 | 11924816 | 23292673 | 3 | 79369491 | C | T | GTTAATTTCTCCGGNGGTCACTCTATCCG |
| 271 | ROBO1 | 17336055 | 24336804 | 3 | 79375985 | G | A | TGTCAGATTCCACCNTTGATGTTCTTTCT |
| 272 | ROBO1 | 1387668 | 23292762 | 3 | 79403096 | A | G | GGAAGTAAATATGTNACTGAATCTATCAT |
| 273 | ROBO1 | 17395749 | 24336869 | 3 | 79431200 | G | C | AAATTCTTCACCAGNAAAGGGATTAACAT |
| 274 | ROBO1 | 17395825 | 24336892 | 3 | 79440079 | A | G | GCAATGTAAAGAGCNTCGTACTAAGMAA |
| 275 | ROBO1 | 6769328 | 23917259 | 3 | 79455526 | G | A | TTTTATAAACCGACCNTATCTTTCGATATC |
| 276 | ROBO1 | • | 46541002 | 3 | 79485023 | A | G | CATTCATTTATTCANTCTCAAAATGTTTA |
| 277 | ROBO1 | 6784911 | 24337076 | 3 | 79494882 | G | A | ATGTAATTTTCTCANCCTGATGAGGAAAG |
| 278 | ROBO1 | • | 46541001 | 3 | 79501791 | G | C | GACCTGATAGAAGTNAGAGATAACCCTGC |
| 279 | ROBO1 | 6798357 | 24337143 | 3 | 79509504 | C | T | CCGCATTTTCACTTNGTAATTCATCAGAG |
| 280 | ROBO1 | 4373036 | 24337174 | 3 | 79519603 | C | A | AGAAAATGGAAAGCNATTAGAGTAAAGA |
| 281 | ROBO1 | 17396958 | 24337219 | 3 | 79525741 | G | A | GTGAAGACGGGGCCNGGCTTATGATAGAA |
| 282 | ROBO1 | • | 46541000 | 3 | 79532529 | T | C | TAAATAATGCATGTNCTAAATTCATAAGT |
| 283 | ROBO1 | • | 46539600 | 3 | 79539676 | G | A | TTACAGACCAAAATNAAATAACATAATTC |
| 284 | ROBO1 | 4452292 | 24337323 | 3 | 79547259 | A | G | TGAUAAAUTGATGCNTAAGCAGAGAAATG |
| 285 | ROBO1 | • | 46540998 | 3 | 79578444 | G | C | AGATGTATAACATGNTTTAAAAATAAACA |
| 286 | ROBO1 | 6770755 | 24267423 | 3 | 79591703 | G | A | CAGGCAAAGCAAATNTTGGTGAATTCTTT |
| 287 | ROBO1 | 7429701 | 23191450 | 3 | 79617522 | A | G | CATTTTGCAGATACNGAAAGTAAGGCACA |
| 288 | ROBO1 | • | 46540997 | 3 | 79643294 | G | A | ATTTTCAAATAACTNGTTGTCTAATTCAG |
| 289 | ROBO1 | 7611237 | 23861629 | 3 | 79654673 | C | T | TAATTGTAGAGCANGAACTATGTGACTA |
| 290 | ROBO1 | • | 46540996 | 3 | 79679725 | T | C | AATAATAATTAATANGCTAAAAACTCAAG |
| 291 | EPHA3 | 7626554 | 24162842 | 3 | 89249704 | A | C | TTATATACAAACTGNGATTTCTTTCCCCC |
| 292 | EPHA3 | 7651124 | 24162849 | 3 | 89287286 | A | G | CCAAAAGTCCATTGNCATTTCAAGCATAG |
| 293 | EPHA3 | 7647240 | 23706498 | 3 | 89326187 | T | A | TTAGTAGAGCAGAGNTTTATCCAGACTGA |
| 294 | EPHA3 | • | 46540864 | 3 | 89333769 | C | T | CCCTATATTCCAATNGTACTGGAACCCAG |
| 295 | EPHA3 | 6551408 | 24644827 | 3 | 89353428 | C | T | TCAAATACGAAAAGNCATACATACATTTA |
| 296 | EPHA3 | 9838512 | 24299971 | 3 | 89367286 | G | A | TTTCTTAGTATAGCNTCGTAATAAATCTT |
| 297 | EPHA3 | 17026816 | 23239496 | 3 | 89380283 | A | G | CCAGTTGAAGACATNAGTGCCCAGTTGCC |
| 298 | EPHA3 | 17800347 | 24300052 | 3 | 89397119 | T | G | CATTTATTTAGAGANGCATCAAGAAAAAT |
| 299 | EPHA3 | 1040017 | 24300195 | 3 | 89412322 | T | G | ATCAGAGCACTTGGNTTGTGACAAGGGAT |
| 300 | EPHA3 | 1512909 | 24300212 | 3 | 89419107 | C | T | TTATCCTGATTTTANGGATGGGAGAATTT |
| 301 | EPHA3 | 1512908 | 24300237 | 3 | 89422824 | G | A | GTGACAGATAGCTCNGTTTCTTCCCTGCC |
| 302 | EPHA3 | 1512911 | 24300243 | 3 | 89431215 | T | C | CAAGAATGAGAATANGGGAAATTTTTAAG |
| 303 | EPHA3 | 2048588 | 24300252 | 3 | 89437883 | G | A | TCATCGTCATTGTCNTCATCATCATCATC |
| 304 | EPHA3 | 7631682 | 23239808 | 3 | 89476833 | A | C | ATTGCAGATTACAANACAAAAATCTTCAA |
| 305 | EPHA3 | 17801001 | 24300274 | 3 | 89497245 | A | C | GGACACACACTTACNTTTCCAAACATATT |
| 306 | EPHA3 | 17801031 | 24300284 | 3 | 89498492 | C | A | AACAGCAATGGATANCTGCTGAGTATAGA |
| 307 | EPHA3 | 17026915 | 23887501 | 3 | 89541302 | C | G | TTTTTTATGGGAAGNCTACATCTTACGAA |
| 308 | EPHA3 | • | 46540883 | 3 | 89598762 | G | A | AGGAATTAAACCAGNTGTAACGGCTATAA |
| 309 | EPHA3 | 12636275 | 23239994 | 3 | 89605728 | C | A | CAAAAGTTGTCCAGNATGTTGTCGCTTGC |
| 310 | EPHA3 | 3762717 | 24300427 | 3 | 89611638 | T | C | ATAGCCATTGATCANAAACTGACTATCAT |
| 311 | GSK3B_NR1I2 | 3814058 | 23254825 | 3 | 121019981 | T | C | CCCTGGGTTTAATGNCAAATCAAGGCAAA |
| 312 | GSK3B | 16830567 | 23896790 | 3 | 121052865 | T | C | GATAAAGCAGTCTTNTCCAATGGAAAAAT |
| 313 | GSK3B | 1719889 | 23254875 | 3 | 121061478 | T | A | GCTACCATTCTCACNTAATCTAGCCAAGT |
| 314 | GSK3B | • | 46540637 | 3 | 121073938 | A | T | ACACTGGACAAGCANAAACTGGCACATAA |
| 315 | GSK3B | 7620750 | 23320928 | 3 | 121125871 | G | A | ATGTTCAAGCATAGNCTTGGCTACTGAGT |
| 316 | GSK3B | • | 46540636 | 3 | 121130705 | A | G | AGATCAATATTCAGNAACAATCAGTTCCC |
| 317 | GSK3B | 17810235 | 24311270 | 3 | 121157741 | C | T | AGTGGCCGGGGAAGNTGTTTTGGAAGCAG |
| 318 | GSK3B | 7644234 | 24311300 | 3 | 121174301 | G | T | CTGTTCTGTGATATNGATTTTTTAAGCAA |
| 319 | GSK3B | 4688054 | 23896924 | 3 | 121216400 | T | C | TGAAAAGGAAAACANAGGGTGGCAGAGGA |
| 320 | GSK3B | 1154595 | 24311339 | 3 | 121219284 | C | T | TTTAACAACTAATANCCAATCCATGAGGG |
| 321 | GSK3B | 6770314 | 23255097 | 3 | 121225411 | G | A | ACAAATTGTGACCCNCGTAGGCAATATGC |
| 322 | GSK3B | 1870931 | 23896950 | 3 | 121239703 | C | G | TACTCAACATGTTANTTCCTCCTAAGCTA |
| 323 | GSK3B | 16830689 | 23255131 | 3 | 121253961 | G | C | GTTCTGATACTAAGNCTGAAAGATTCATG |
| 324 | GSK3B | 2199503 | 23896976 | 3 | 121261179 | T | C | TCTGAAATCTTAGTNCAACTTCCTCACCC |
| 325 | GSK3B | 334535 | 23255219 | 3 | 121274994 | T | C | TTTCAACTACACTTNTTACCCATCTCTCA |
| 326 | GSK3B_LOC3 | | 46540634 | 3 | 121294157 | G | A | AACATAAATTATACNGTGGCAGGTTGGTA |
| | 89143 | | | | | | | |
| 327 | SEMA5B | 7631746 | 23904005 | 3 | 124107946 | 6 | C | GATCCTTATGTGCCNATTCGTACCACCCT |
| 328 | SEMA5B | 2276778 | 23904039 | 3 | 124125280 | G | A | ACA6CAGAAGCCGCNATGCTGTTTCTGAA |
| 329 | SEMA5B | 9835012 | 23268599 | 3 | 124131938 | C | T | GCCACGGTGTTCATNTGGACAGAAACAAC |
| 330 | SEMA5B | 10934622 | 23904124 | 3 | 124142519 | A | G | TTATCCCATCAAAANTCATTCATCACAAC |
| 331 | SEMA5B | 4677981 | 23268699 | 3 | 124148245 | G | C | ATTAGGTACGCAGANGAAACTCTTGTTCA |
| 332 | SEMA5B | 6764954 | 24361038 | 3 | 124168761 | A | G | TTTAAATGTGACAANTCTTGGAAGAATTA |
| 333 | SEMA5B | 1470399 | 23268815 | 3 | 124181451 | C | T | TGACTTTATTTTCCNCTAAATGTGTGTCC |
| 334 | SEMA5B | 10934633 | 24320632 | 3 | 124192662 | G | A | ACTGCACAGATGGANGACCCAAACCAGCA |
| 335 | SEMA5B | 16833766 | 23269126 | 3 | 124203397 | A | G | ACTCTAAATTCTACNCGATAAGGTGCATG |
| 336 | SEMA5B | 2034177 | 23269150 | 3 | 124211779 | A | G | GGTAGGATGGGACANATCATGGAATTTGG |
| 337 | SEMA5B | 7629297 | 23269178 | 3 | 124219868 | A | T | TTTCTCCCGAGCTGANGAGTATGTTTTCC |
| 338 | LOC442088_P | 17751931 | 24291218 | 3 | 128239188 | G | A | TAGATGCTTCCCAGNCGATTCTCCCGTGA |
| | LXNA1 | | | | | | | |
| 339 | EPHB1 | 719893 | 24392684 | 3 | 135991269 | G | A | GTGGGTTGGCCTTCNATCCTCTAGCTGTC |
| 340 | EPHB1 | 17695812 | 24392734 | 3 | 136010621 | A | G | TTTTCACTATTTCANTCATGGCCTAGTCT |
| 341 | EPHB1 | 4955455 | 23366127 | 3 | 136015335 | A | G | TTTGCATGATGACANTCAGTGATCACAGT |
| 342 | EPHB1 | 1399708 | 24648031 | 3 | 136037585 | C | T | TTCTGTGGACACTCNATTTGACGGCAGAA |
| 343 | EPHB1 | 2292659 | 23711454 | 3 | 136050152 | C | G | TAAAATAGAAATAANTTTTTATTGCCATT |
| 344 | EPHB1 | • | 46540505 | 3 | 136061921 | T | C | GGGCCATCTGTTCCNAGGCTGGTTGGAGA |
| 345 | EPHB1 | 1515367 | 23366237 | 3 | 136075138 | G | T | GTCTGTCATGCACCNAGTTGATGGTCTTC |
| 346 | EPHB1 | 6439536 | 24392908 | 3 | 136101542 | A | T | AGGTCATTGTGTCTNTGGTGATTAAACCA |
| 347 | EPHB1 | 36206 | 24393028 | 3 | 136115385 | C | T | CCAGTGCAGTTCCANGTCTCAGTGTATAT |
| 348 | EPHB1 | 36193 | 23960461 | 3 | 136121534 | T | C | CCAGGCTCCATACANCTGCCATTTTATTC |
| 349 | EPHB1 | 36183 | 24393111 | 3 | 136128099 | C | T | AGTAGGAGGAATGCNGGTTGAAAGGGATG |
| 350 | EPHB1 | 36170 | 23366794 | 3 | 136134783 | C | T | TCTTGGTCCTAAGANGTCTGTCTGCAGCA |
| 351 | EPHB1 | 3772657 | 24620546 | 3 | 136141352 | C | T | GGCAGCCATCTCCCNGGATTGGGCAATGC |
| 352 | EPHB1 | • | 46540509 | 3 | 136147631 | A | G | CAGTGAAGGTCTAGNGATTCTCCCCTAAA |
| 353 | EPHB1 | 7644369 | 23673466 | 3 | 136153222 | C | T | GCTGCAGATGAGAGNTTCTCCCAGGTGGA |
| 354 | EPHB1 | 6785203 | 23673474 | 3 | 136159208 | T | C | CTGTCTGGGTGTTANAGAAACAAACATCC |
| 355 | EPHB1 | 936325 | 23673483 | 3 | 136164992 | C | G | ATGATGGTTATAACNTGGGTATGGAGCAC |
| 356 | EPHB1 | 17282355 | 24620553 | 3 | 136179029 | C | T | TCTGGCAGACTTTCNTTCAGTGGCCAGGA |
| 357 | EPHB1 | 36105 | 24620560 | 3 | 136185639 | A | G | TATTTTCACAGTAANAGTCACTAAAGGGG |
| 358 | EPHB1 | 9289488 | 23673491 | 3 | 136193002 | G | A | TCCTGCCTCAGAGTNGAGATCTGCAGTTT |
| 359 | EPHB1 | 36073 | 24620574 | 3 | 136202852 | C | T | CATCTGGGAGGTTGNTAACAGTGAGCTCT |
| 360 | EPHB1 | 41182 | 23673499 | 3 | 136208979 | G | A | AGCCCGAAATAATGNCAAGTGGATTCATG |
| 361 | EPHB1 | 36059 | 23673504 | 3 | 136215430 | C | A | AAGTCTGATGGAAANAGTGATTCTGAAGG |
| 362 | EPHB1 | • | 46540492 | 3 | 136226032 | A | G | AAACACGGATGGACNTC0CCAGAATTTAA |
| 363 | EPHB1 | 9815942 | 24620579 | 3 | 136237021 | A | C | ATGGAATTGGGAAANGTGAATTACCTTTA |
| 364 | EPHB1 | 36041 | 24620580 | 3 | 136248996 | G | A | CATTGGCATCTGCANGTCTTCTTGTTAAT |
| 365 | EPHB1 | 2030737 | 24648172 | 3 | 136292075 | C | T | CTTGCAAAGTGCTANATCCACTATCACTG |
| 366 | EPHB1 | 1554675 | 23367146 | 3 | 136309615 | C | T | ATTGCCGAAGTATANGCCCAGGGACTCAC |
| 367 | EPHB1 | 16842632 | 23367222 | 3 | 136320313 | G | A | ACAGAATTGCACAGNGAGGCCCTAGCAAC |
| 368 | EPHB1 | 7625747 | 23367267 | 3 | 136328391 | T | G | TAGGTTCCATGACCNCACGGCTTTCACAG |
| 369 | EPHB1 | 11713704 | 24393645 | 3 | 136336460 | C | T | TACCCTGCTATTCCNGAACCTGCCCATCT |
| 370 | EPHB1 | 3772648 | 23961027 | 3 | 136346310 | A | T | GGCATGAAGCCCTCNCTTTTTATCCCATT |
| 371 | EPHB1 | 1501206 | 23961119 | 3 | 136362452 | A | C | AAATGTCTGCTGGCNAAGATGTGGTCCCC |
| 372 | EPHB1 | 1875371 | 24394023 | 3 | 136383645 | G | A | AGACACTCCTGCAGNGAACCAATTTTTCT |
| 373 | EPHB1 | 4894286 | 23368000 | 3 | 136390877 | A | T | ATTCATTTGAAAAGNGCTTGGATGGTTTA |
| 374 | EPHB1 | 3821502 | 23368064 | 3 | 136395265 | A | G | TTAAAGGGTCAGTCNTTGAAACATTGCTG |
| 375 | EPHB1 | 6439561 | 24394231 | 3 | 136401537 | G | C | TCTGGGATGCAGGTNGTGGTTGCCTTGCA |
| 376 | EPHB1 | 1875370 | 23368228 | 3 | 136426983 | A | G | GTTCCTGTGATGCCNTCAAAATGAGTAAG |
| 377 | EPHB1 | 9828343 | 23368318 | 3 | 136432601 | C | A | AAGACCTGACTACCNCTGCGGGATTCCCT |
| 378 | EPHB1 | 17712106 | 24394514 | 3 | 136446103 | C | G | CATACAATTCAAACNTGGTCATTGTCATT |
| 379 | EPHB1 | 17712383 | 24394605 | 3 | 136464893 | A | C | GCACCATCCAGAGCNCTCCAAGGACACTC |
| 380 | EPHB1 | 13072079 | 24394675 | 3 | 136470303 | T | A | GTCAAGCAAAGGAGNGTGTCTTATTCGTG |
| 381 | NCK1_TMEM | • | 46540496 | 3 | 138061897 | C | A | TAGAGTAGCTGTTGNCCAGTTTCATGGTC |
| | 22 | | | | | | | |
| 382 | NCK1 | 9818792 | 23288753 | 3 | 138072018 | C | T | TATTCCAGCTGTATNGCAGAAATATTTTA |
| 383 | NCK1 | 1865149 | 23288793 | 3 | 138078308 | G | A | GCTGTAAGGCTACANTGAGAATTTACGAA |
| 384 | NCK1 | 12488365 | 23288897 | 3 | 138089176 | C | A | ACTTCTTTAGCATANAATTCAGATACAAC |
| 385 | NCK1 | • | 46539382 | 3 | 138104136 | T | A | TTTTGTGTCCTAACNTGATCTATCCTGGA |
| 386 | NCK1 | 1347209 | 23288987 | 3 | 138109395 | G | T | ACCTATTGTCCTCANTGCTACAGGTATTA |
| 387 | NCK1 | 4678275 | 23289141 | 3 | 138136381 | G | A | AGAGAATTGGAGTCNGTTAAGCAATGGGT |
| 388 | NCK1 | • | 46540495 | 3 | 138145301 | G | A | AGGAAGACAGACAANTAAACAAGTACAAT |
| 389 | MGC34923_N | 835637 | 24648412 | 3 | 138155855 | T | A | AGAGACACAGTGAANTCCAACTAAATGTC |
| | CK1 | | | | | | | |
| 390 | MRAS | 4678260 | 23673640 | 3 | 139572211 | C | T | CTGAAATCCCCACTNGAATGAGAGGAAAT |
| 391 | MRAS | 1209710 | 24620657 | 3 | 139589396 | T | C | AGTTCTGGGGATCANGGGCCTTGTGAAAA |
| 392 | MRAS | • | 46540488 | 3 | 139606913 | T | A | GATACTTGAATTATNGTGCGCCTGTGAGC |
| 393 | MRAS | 1871516 | 24144410 | 3 | 139615132 | C | T | CCTCATTTGTCAAANTGGATTGATGATAA |
| 394 | PAK2 | • | 46539746 | 3 | 197969334 | T | C | TATCTCATCCTGGTNTCTTCTCTCCATAT |
| 395 | PAK2 | • | 46539745 | 3 | 197982271 | G | A | CCATGGCAGTATATNGAGCTATTTTCTCA |
| 396 | PAK2 | • | 46539744 | 3 | 198007519 | T | C | GGGACATAAAGAAANGTTGAGTGCACGAC |
| 397 | PAK2 | • | 46539564 | 3 | 198047259 | A | G | AGTTCGAAAGAAGANGCAAGCGCTGGCCT |
| 398 | PAK2 | • | 46539381 | 3 | 198050436 | T | A | TTGAAAGTGACCTTNGGCCATGGACTGTT |
| 399 | ABLIM2 | 10008860 | 24307862 | 4 | 8087665 | G | A | CAGCACTGGCTGTGNACTGTACCCGTGGG |
| 400 | ABLIM2 | 4696726 | 23970035 | 4 | 8110703 | A | G | CCCAAAGCAAAGTCNTTAATAATATTCAC |
| 401 | ABLIM2 | 16841552 | 23894039 | 4 | 8127808 | C | T | GGGCCTCTTTGCCGNGAGGCATTTTGTGC |
| 402 | ABLIM2 | 17795113 | 24308070 | 4 | 8136227 | C | T | CCGATATCAAGTGTNGTGTGTCATCTCCC |
| 403 | ABLIM2 | 7690495 | 23382521 | 4 | 8164833 | G | A | GGGACAAGAGCAATNGCCGTACCTCAGGC |
| 404 | ABLIM2 | 6842276 | 24405693 | 4 | 8193205 | C | T | TCTGGGCTGTTCTTNGCGGAGAACGGAAA |
| 405 | ABLIM2 | 17178920 | 24405714 | 4 | 8201634 | C | T | GGGACGGAGCAGGGNCCACTGAGTCAACC |
| 406 | ABLIM2 | 2386113 | 23250964 | 4 | 8225125 | G | A | CCTTTTCAAATCGTNTTGCACACCTTACC |
| 407 | ABLIM2 | 13116608 | 24308278 | 4 | 8231961 | T | C | AATAACAAAAATGANTGCTGGTGTCGTTA |
| 408 | ABLIM2 | 4696763 | 23251174 | 4 | 8238737 | G | A | CTTATTTTACAGACNCAAAAGCTCACCAG |
| 409 | ABLIM2 | • | 46544376 | 4 | 8256394 | G | C | CTTTGTTTGTCAGTNGCCTGTTGCCCACC |
| 410 | ABLIM2 | 16841849 | 23251340 | 4 | 8262222 | G | A | GACTCTCCACCTTCNGGCACAGAGGGCAC |
| 411 | SLIT2 | 643562 | 24366326 | 4 | 19925417 | C | T | ATGGATTTATGGTTNAACTGGTCTAATCC |
| 412 | SLIT2 | 7692329 | 23331966 | 4 | 19942571 | C | T | AGTGTTTCAGAGGCNAGTGGTGTTTTGTG |
| 413 | SLIT2 | 587032 | 24366512 | 4 | 19948042 | A | G | AGGAAATAGGGTGTNGGTTTGCTATAAAG |
| 414 | SLIT2 | 16869475 | 23940246 | 4 | 19956421 | G | A | GACTTTTCTACCCANCCTCTTGTCACAAA |
| 415 | SLIT2 | 11945395 | 24366777 | 4 | 19966493 | A | G | TTTAATTTAACAATNAGACATAAAACATT |
| 416 | SLIT2 | 10516351 | 23940403 | 4 | 19975144 | C | T | TTTAAGAAATAGTTNGTATGTTTACTAAT |
| 417 | SLIT2 | 13140329 | 24366927 | 4 | 19984191 | C | T | TCTTTTGATCATTANGTTTCCTGTTTTCA |
| 418 | SLIT2 | 1344849 | 23940570 | 4 | 19995818 | T | C | AAATAAAGGATTTCNGTTGGATTTCTAGC |
| 419 | SLIT2 | • | 46541954 | 4 | 20033389 | T | G | TATTGTTTTCCATTNCAAGGTGTGTATTC |
| 420 | SLIT2 | • | 46544266 | 4 | 20046412 | G | A | AACACCTAAAGTAGNTGAAGAGGTTAATG |
| 421 | SLIT2 | • | 46544264 | 4 | 20062313 | A | C | TGGGGTGACAGAAGNATTCTGCATCATGA |
| 422 | SLIT2 | • | 46544263 | 4 | 20081301 | C | T | CTTTTTTGAACCTCNGTTGCTGGGGCATT |
| 423 | SLIT2 | 1995982 | 23940671 | 4 | 20101043 | C | G | CCCAAATTTTTAGANTACAGCCAAAGTAA |
| 424 | SLIT2 | 17610209 | 24367359 | 4 | 20110053 | C | T | CTGCCTTTTGCCTThGGTGAACTGGCTAG |
| 425 | SLIT2 | 10516355 | 24367403 | 4 | 20117672 | C | T | TATCTGAACCCGTANAAAGATTTAGTAAT |
| 426 | SLIT2 | 6834573 | 24367463 | 4 | 20123113 | G | C | CTACTTCAGTAATTNAATGTGCTTGCAGA |
| 427 | SLIT2 | 17610727 | 24367503 | 4 | 20129891 | A | G | AAATGTTAAAAGTANATTTTGTCTCATTG |
| 428 | SLIT2 | • | 46541810 | 4 | 20140182 | G | C | TTCAAGTTTAAGGTNCTCAATATTTTTAA |
| 429 | SLIT2 | 625614 | 24367723 | 4 | 20153069 | G | A | AGAATGGCCTCTCTNTTGCAGATATTCAC |
| 430 | SLIT2 | 1355512 | 23941121 | 4 | 20159363 | G | A | CTAAAGCATTATATNCCGCCTATACATTT |
| 431 | SLIT2 | 490478 | 24368111 | 4 | 20174753 | A | G | GCTGTATTAACCAANTTTGATAAATAGAA |
| 432 | SLIT2 | • | 46544261 | 4 | 20191104 | C | T | AACTGCAGTTAAGTNAGGGAGTTAAGTCA |
| 433 | SLIT2 | • | 46544260 | 4 | 20192610 | T | C | ATAAGTGGACTTGANTTACCTAATGTTTT |
| 434 | SLIT2 | 16869714 | 23334694 | 4 | 20204956 | G | A | CGCAAACTTCTTATNAGATTCTGATTCCA |
| 435 | SLIT2 | 3775816 | 24368395 | 4 | 20211986 | T | C | TGCTTATATGGATCNGACTACCCTGTCCT |
| 436 | SLIT2 | 606524 | 23335104 | 4 | 20230636 | A | G | CTAAAAAAAATAAGNTAACTATCCTGACG |
| 437 | SLIT2 | 1463463 | 23941788 | 4 | 20237953 | A | G | AGTTTTAAAGCAGCNAAGTCATGTTTCTT |
| 438 | SLIT2 | • | 46544259 | 4 | 20243919 | A | G | GAGTTTCTTTCAGTNGATCAACAACGCAA |
| 439 | SLIT2 | 2290749 | 23335283 | 4 | 20244958 | A | G | TCACAGTACATCCANTTGAGAAGGCCTCC |
| 440 | SLIT2 | 10516358 | 23335385 | 4 | 20253376 | A | T | TCTATGAAACAACGNTAAACCACTTATTC |
| 441 | SLIT2 | 9992591 | 24368931 | 4 | 20270015 | C | A | GTAAAAATCAAATTNTGTGAAGCTGCTAA |
| 442 | SLIT2 | 7683520 | 23335563 | 4 | 20279428 | C | G | CAGGTGCAATGGATNTACTCTTAATCCTC |
| 443 | SLIT2 | 17621372 | 24369081 | 4 | 20292755 | C | T | ACTCAGAATAATTCNTGATTTTATGATAG |
| 444 | SLIT2 | 936359 | 24369202 | 4 | 20304528 | C | A | TCTGAGATTCAGAANGCAGCAAACTTATA |
| 445 | EPHA5 | 4860651 | 24290719 | 4 | 66017061 | T | C | TTTTGGACAATTTCNAATACACTGCATAA |
| 446 | EPHA5 | 4860652 | 24290818 | 4 | 66025401 | G | A | TCGGTATGAAGGAANAGAATGCTGAGAAT |
| 447 | EPHA5 | 12651591 | 23880273 | 4 | 66033080 | C | T | TTCGGTCTTATTTTNGGGTAAATTTATAC |
| 448 | EPHA5 | 7656497 | 23880359 | 4 | 66039430 | A | G | AGTGTGATGCTTAGNTACAAAAATTTTGG |
| 449 | EPHA5 | • | 46543786 | 4 | 66048199 | C | A | AAACTCCATCCTATNTAACTGCCCTGTTT |
| 450 | EPHA5 | • | 46543785 | 4 | 66057493 | A | G | GAAATTACTTCAGGNCAAAGTGGAAGGGA |
| 451 | EPHA5 | 17086125 | 23226131 | 4 | 66064957 | A | C | GACGAATTCTACAGNGACCTGAAAGCTAT |
| 452 | EPHA5 | 7697278 | 23226262 | 4 | 66071892 | C | T | TCCTCAGAAATTCCNTCAAAATTTACATC |
| 453 | EPHA5 | 17752325 | 24291351 | 4 | 66074122 | C | T | TGTTAGCTATTACTNTTAATTTTATCATA |
| 454 | EPHA5 | 1451174 | 24291392 | 4 | 66086658 | G | C | TAATAAATTATGATNTGCTCTCTAATTAT |
| 455 | EPHA5 | 10517937 | 23226603 | 4 | 66096086 | A | C | GTGCTGTCTTTTCANCTGCAAATTCCTAG |
| 456 | EPHA5 | 10030406 | 23227041 | 4 | 66112025 | T | G | ATATTATATAAAGANTTTTCTGCTTTTAA |
| 457 | EPHA5 | 4406062 | 24651593 | 4 | 66131088 | A | G | TAATTTGTGATTCANTGTTTGTACTTCTG |
| 458 | EPHA5 | • | 46542115 | 4 | 66162699 | C | G | GCCAGCTGAGGAAANATGGAACACCTAAT |
| 459 | EPHA5 | 6551929 | 24651631 | 4 | 66173956 | A | G | CATTTCTTGGTGAANAAAATAGAACTATA |
| 460 | EPHA5 | 7684022 | 23716975 | 4 | 66218467 | G | A | TGAAAATCCTAGAANTAATTCAAAACAAT |
| 461 | EPHA5 | 6551935 | 23716989 | 4 | 66242150 | G | A | ATTTAAAATGCTTGNTGTTTTGAGAAACT |
| 462 | EPHA5 | • | 46541858 | 4 | 66259270 | T | C | TTTAGGGTGATACANATATACTTTTATAG |
| 463 | EPHA5 | 4406064 | 24168485 | 4 | 66280307 | G | A | ATAAAGGAGCATATNTTTTTCTACAGCAG |
| 464 | EPHA5 | 4597887 | 24168510 | 4 | 66305784 | A | G | GTAAATTGAAAAGTNACATAATTGGTACT |
| 465 | EPHA5 | • | 46543765 | 4 | 66329908 | G | T | TTTGACTGGAGGAANAAAATAGAGCAAAA |
| 466 | EPHA5 | • | 46543764 | 4 | 66345280 | A | G | CTCTGCAGAAAAAGNTATGATGCTTAACA |
| 467 | EPHA5 | 6551939 | 24651790 | 4 | 66352249 | G | A | ACTTAATAATATTCNTTCACTAAACACAA |
| 468 | EPHA5 | 4510507 | 24168571 | 4 | 66371004 | A | G | ATGACTGGTGGGATNAATTGAATTTATTT |
| 469 | HNRPDL_MA | 10516674 | 23146012 | 4 | 83715941 | T | C | GCTACCTCTTCTTGNAAAGGACCTATTTA |
| | SA | | | | | | | |
| 470 | MASA | 17351082 | 24235863 | 4 | 83732925 | G | A | TGCAGGAGTTTGTANTCTCAGGGAGCTGC |
| 471 | MASA | 3821975 | 23835889 | 4 | 83741474 | C | T | GGGACAAAGTTCCTNTCTACTCCCATTGT |
| 472 | UNC5C | 2241743 | 24340726 | 4 | 96448702 | G | A | AGCATTTATTTCTGANGACAAAAGCAATCT |
| 473 | UNC5C | 11097458 | 24340746 | 4 | 96456545 | T | C | CACTCACTAGTTCTNATGCCATTTCTAAG |
| 474 | UNC5C | 10026552 | 23919930 | 4 | 96461977 | C | T | ACAGGCTTTAATTTNCTAAAAGTCTAGAC |
| 475 | UNC5C | 767824 | 23919975 | 4 | 96466214 | G | A | CCAATGGGACATAANCCCTGGGGAAAGAC |
| 476 | UNC5C | 11737434 | 24340874 | 4 | 96479793 | T | C | TNTTTTCAATCATGNCTTTTTTCTGTTTA |
| 477 | UNC5C | 9307155 | 23298575 | 4 | 96486699 | C | T | ACTCTAAGTATTCCNAACATGAAAGGCTC |
| 478 | UNC5C | 7676599 | 24341179 | 4 | 96500878 | C | G | CATAAACTCTAATGNATCTAATTTATTTC |
| 479 | UNC5C | 17023302 | 23299004 | 4 | 96510877 | G | T | GTGATCTCGAAGGANCCCATGCTTTGCTG |
| 480 | UNC5C | 4699414 | 23920379 | 4 | 96519220 | G | C | AGGTTCTGTCTCGTNGCATTGTATTCCTT |
| 481 | UNC5C | 4699837 | 23299259 | 4 | 96527617 | T | C | ATGACTGACTGTACNATGAGCCACATACT |
| 482 | UNC5C | 6846193 | 23299355 | 4 | 96538418 | C | T | GTCCTATGACTGCANTGGTCCCCAAAAAG |
| 483 | UNC5C | • | 46543454 | 4 | 96555731 | A | G | AGGACACGGGGTCANTTAGGGCAATAGTG |
| 484 | UNC5C | 2865431 | 23300063 | 4 | 96566251 | C | T | GGTATATAGCAGTGNCTTAGTAAATGCTG |
| 485 | UNC5C | 17432897 | 24342072 | 4 | 96573500 | A | T | TTGCCTGAACCTTANGCTTTGGGATTCAC |
| 486 | UNC5C | 10049501 | 23300250 | 4 | 96582519 | A | G | CGTGACAGGATCCTNAGGGGCAACTCTAT |
| 487 | UNC5C | 4699416 | 24342362 | 4 | 96598100 | A | G | AAATTTTTAACTAANAAGTTCTCTGTTGT |
| 488 | UNC5C | 17023543 | 23300853 | 4 | 96605013 | A | T | AATGGTAATGAATGNTCTTCAATCAGTAT |
| 489 | UNC5C | 4699420 | 24342582 | 4 | 96612699 | G | T | CAGAGAGATATCTANTAAGGGTAAAAAAG |
| 490 | UNC5C | • | 46542051 | 4 | 96628453 | T | C | GTACTTGACATTCANTTTAACGTGCTTAA |
| 491 | UNC5C | 17023624 | 23301666 | 4 | 96642908 | C | T | ACCTGCTGGTTTTANGGAGAAGGCAGGGG |
| 492 | UNC5C | 17023642 | 23921707 | 4 | 96649574 | A | G | TTTCTCCATAGGTTNTATCTTGACAGAAA |
| 493 | UNC5C | 6845966 | 23301954 | 4 | 96656271 | C | T | TTGCTTTAAATATANAGCAAGTAGAGCTC |
| 494 | UNC5C | 9998018 | 23302266 | 4 | 96663585 | T | C | TGATTTTGTTCCTANTGTGTCGATGTGCG |
| 495 | UNC5C | 17023728 | 23302457 | 4 | 96670546 | C | T | GATAACTCCCTTCANTCCCTAGAAACTTA |
| 496 | UNC5C | 17023753 | 23302594 | 4 | 96673797 | A | G | ATTTGTCCAGGTTANATCTATAGAGTTCT |
| 497 | UNC5C | 9307158 | 23302709 | 4 | 96687694 | G | A | GCAGACATCAAACANTTCAAGGGACTCAG |
| 498 | UNC5C | 9307159 | 23302998 | 4 | 96699064 | C | T | TGAATACACCATCANTGTGGGTGACATGG |
| 499 | UNC5C | 1483748 | 24344077 | 4 | 96710351 | G | A | TAAGAGATGTAAAANGAAATGAATAGAAG |
| 500 | UNC5C | 4699266 | 24344240 | 4 | 96721087 | G | A | AAATAATTAGTAATNTATGCAGACGTGTC |
| 501 | UNC5C | 4103380 | 24344316 | 4 | 96727783 | G | C | TTGACTTCCTGTAANGTGTATGGTGGTAT |
| 502 | UNC5C | 4444836 | 23304197 | 4 | 96745829 | A | G | CACTGGAAGTTACANTCATCATTATGTGG |
| 503 | UNC5C | 9996606 | 23923090 | 4 | 96751972 | C | G | TTATTTGCAAAAAGNCTATCATAAGATGA |
| 504 | UNC5C | 265070 | 24344895 | 4 | 96758825 | C | T | CTCAAAGACCACCCNATGTCATTTTCTAC |
| 505 | UNC5C | 265011 | 23304813 | 4 | 96770673 | A | T | AAATTAGAATGTGCNTAGCGTTTTGTCGT |
| 506 | UNC5C | 1485604 | 24345179 | 4 | 96779400 | U | A | GAATTAAAACACCANACTCATAAGCACTG |
| 507 | UNC5C | • | 46543442 | 4 | 96787710 | C | T | CACATATTTCAGCCNTCAACTCCTAATTT |
| 508 | UNC5C | • | 46543441 | 4 | 96794340 | A | T | ATATAAAAACGTTGNAGTGAACTGAAATA |
| 509 | UNC5C | 10016515 | 24345428 | 4 | 96800167 | T | A | GCAAGGGATAACACNAAATGGTGCAATTA |
| 510 | UNC5C | 10021966 | 23305389 | 4 | 96807271 | T | C | ACTTGATTTTTTTTNCTGAAAATTAGGTA |
| 511 | UNC5C | 17386118 | 24345672 | 4 | 96813780 | G | A | GATAAGAATAAAACNGCACAGCAACAATT |
| 512 | UNC5C | 11097469 | 23305766 | 4 | 96823244 | G | A | GCTGCTTCTGCAAANCCATGACCTAAATC |
| 513 | PPP3CA | • | 46543373 | 4 | 102299661 | T | C | TGGCAGGAATCAGANCGTGAATACACTTA |
| 514 | PPP3CA | • | 46543372 | 4 | 102300025 | T | C | GAAGACTGAATAATNCCATGGATATTTAG |
| 515 | PPP3CA | 2037773 | 24313586 | 4 | 102315795 | T | C | AAATTTGTACCACANTCCACATTCATTAA |
| 516 | PPP3CA | 2695196 | 24313656 | 4 | 102325135 | T | C | AATTTGAATAAAACNTCTTGATGAAATGA |
| 517 | PPP3CA | 1405686 | 24313707 | 4 | 102337045 | G | A | CCTTTCATGTAAGTNATTGAGATGGTGAG |
| 518 | PPP3CA | 2695205 | 24313752 | 4 | 102343941 | G | C | CATGCAACCAGTAANTGACAGATCTCACT |
| 519 | PPP3CA | 17030739 | 23258274 | 4 | 102353990 | G | A | CCCAGAGGTTCATTNCAATTTTTGGCCAA |
| 520 | PPP3CA | 17030740 | 23258317 | 4 | 102363455 | T | C | AGATTTGTTTGGTANATATTAGCTATAAA |
| 521 | PPP3CA | 2732504 | 24313826 | 4 | 102367005 | C | T | TGTGTGCTAAATAANTGAATGAGTGAAAA |
| 522 | PPP3CA | 10516473 | 24314026 | 4 | 102389686 | G | T | ACATGTCAATGGGTNTGACGCTGACACTT |
| 523 | PPP3CA | 1506801 | 23258780 | 4 | 102400788 | C | T | TGTTACAGATGTGANACAGGGTAGATTCT |
| 524 | PPP3CA | 2732514 | 23258854 | 4 | 102416020 | T | C | ATTTTTACTTTTCANTGTGGCTGATTTTA |
| 525 | PPP3CA | 10516472 | 23898948 | 4 | 102422767 | T | C | CTCCTTAGGACACANTTGAATGCCTAGAA |
| 526 | PPP3CA | 2695206 | 24314190 | 4 | 102429540 | T | A | TGGTTGAATAATCANTGTTCTGGCCAAAT |
| 527 | PPP3CA | • | 46543368 | 4 | 102441793 | G | A | TTAAAUAGATCACANAAGCATGTAGCATA |
| 528 | PPP3CA | • | 46541970 | 4 | 102447655 | G | A | TGCGTAGAGAAGATNCTCAGTCAGCAGCA |
| 529 | PPP3CA | 950177 | 24314401 | 4 | 102455751 | G | A | TGGCACAATCTCTCNGGATTCCTGATTCA |
| 530 | PPP3CA | 2659506 | 23259565 | 4 | 102466242 | C | T | TTACTTCGAGAAAANGGAAAGGGAAACAC |
| 531 | PPP3CA | 10005338 | 24314497 | 4 | 102474032 | A | G | CATCTCTAAGTTGCNTTCCACTTTTTAGA |
| 532 | PPP3CA | • | 46543366 | 4 | 102479933 | C | T | AGATGCAATGTCTTNGCAAGTCCAGACAC |
| 533 | PPP3CA | • | 46541879 | 4 | 102484737 | A | G | CCAGCAAATGGAAANTGAACAATTTTATA |
| 534 | PPP3CA | 2659526 | 23899634 | 4 | 102507162 | A | T | AAAGAGCCATTTCANTAGAAAAGATCTGG |
| 535 | PPP3CA | 3804397 | 23899731 | 4 | 102517829 | A | G | TTAAGTATAGGACANGACCTAAGGACAAT |
| 536 | PPP3CA | 13115754 | 23899828 | 4 | 102525885 | G | C | TTGTACTAAACACANTATGAGCATCATTT |
| 537 | PPP3CA | 2028475 | 23899959 | 4 | 102533659 | C | T | GTAAGAAAGAGATANGCTACTGTTGTCGT |
| 538 | PPP3CA | 2583410 | 23900086 | 4 | 102539377 | A | C | GATCTTCCACAAGCNAGGTAACATCATCA |
| 539 | PPP3CA | 2850338 | 23900151 | 4 | 102546050 | U | C | TGCAATTTATACTANTAACAAAAGTGAAC |
| 540 | PPP3CA | 11937437 | 23900239 | 4 | 102553061 | C | I | AATTAGTAAAATCTNTGGGAGTTTATTCT |
| 541 | PPP3CA | 2850975 | 23900276 | 4 | 102558817 | A | I | CAGGTATTTTGTATNTATGAGACTAGAGG |
| 542 | PPP3CA | 2583406 | 23900373 | 4 | 102570898 | C | T | ATTACTTTAGAGCTNTACTTTTAAATACT |
| 543 | PPP3CA | • | 46543393 | 4 | 102579164 | T | U | CCTAAATGTCATTCNGGATTGTACTGTAT |
| 544 | PPP3CA | 2044041 | 24315813 | 4 | 102592547 | T | A | ACTGACAACATTAGNGTAGATATTTCAAC |
| 545 | PPP3CA | 1031033 | 23900488 | 4 | 102606014 | T | G | CTGTATATGTATTANAAATGTTGGGCATA |
| 546 | SEMA5A | 17235366 | 24324380 | 5 | 9092288 | C | T | CTAAAACTCTGGCANTCTGGGCAGTTGAT |
| 547 | SEMA5A | 1354570 | 23274439 | 5 | 9112223 | T | G | CAGAACCATCTCAGNTAAGTCTGATGAAA |
| 548 | SEMA5A | 16881913 | 23274584 | 5 | 9125053 | A | G | CTACATGGAAGTAGNACCATGCTCCCAGG |
| 549 | SEMA5A | 16881928 | 23274676 | 5 | 9131372 | G | A | TAACAATTGAAATCNGTGACCTTGAGCAG |
| 550 | SEMA5A | • | 46559485 | 5 | 9137076 | G | T | CAACACATACAGCANTTTAGTTACCTTGA |
| 551 | SEMA5A | 253633 | 24324813 | 5 | 9144138 | G | C | ATCATTTTTACCTTNAGTGCTTAGGCTGG |
| 552 | SEMA5A | 3777246 | 23907645 | 5 | 9163584 | C | T | TCCAACCTAGAAAANGGCGATGGTGTTGG |
| 553 | SEMA5A | 253660 | 23275441 | 5 | 9177406 | A | G | ATTGAAGAAGGGACNGCTTCTTGCCAAAT |
| 554 | SEMA5A | 10062083 | 23907732 | 5 | 9183973 | A | C | GTATAATGTCAAAANTGTTGAAITTAAAT |
| 555 | SEMA5A | 16882062 | 23275690 | 5 | 9190499 | A | G | ACTTTGTCTAAATGNAAGAGTTCATACAC |
| 556 | SEMA5A | 12189232 | 23907866 | 5 | 9201278 | A | G | ATTCCATGGTGTTCNGAAAATATTAGGGT |
| 557 | SEMA5A | 3797914 | 23276167 | 5 | 9213984 | T | G | ACACAAAACAGTAGNAGATTCCATTGGAA |
| 558 | SEMA5A | 40718 | 23908049 | 5 | 9226880 | A | G | TCAAACAAGGTCTCNTTAACACATTGCAT |
| 559 | SEMA5A | 42354 | 24325718 | 5 | 9239777 | A | G | TGTGCTCAATATCANATTCTTTTTTAGAT |
| 560 | SEMA5A | 1806088 | 23276628 | 5 | 9247903 | T | C | TTCATTATGCTACANGGACTGGCTTCCAT |
| 561 | SEMA5A | 40681 | 23908411 | 5 | 9256073 | C | T | GGTCTCATAATTAGNTTTAAAATGGGGAA |
| 562 | SEMA5A | • | 46559489 | 5 | 9268306 | A | C | TAACAATGGAGATGNTATGGCTGATATAG |
| 563 | SEMA5A | 17254539 | 24326225 | 5 | 9278088 | C | T | CATCATCACAGTGANGCTTGTGCAACAGC |
| 564 | SEMA5A | 40660 | 23277359 | 5 | 9285330 | T | C | ATGTTCTGTGGATCNCTCAGGTATCTCTT |
| 565 | SEMA5A | 1805954 | 23277462 | 5 | 9292922 | G | A | AAAGAGTCATTCACNGTGGCTTTCATTGG |
| 566 | SEMA5A | 1805948 | 24326528 | 5 | 9310053 | A | G | CATCTTGCATATCTNTGTGATGTGAATAA |
| 567 | SEMA5A | 4702617 | 24326614 | 5 | 9320171 | A | G | CTCTGCCCAGCTAGNAAGTAGCAGAATCA |
| 568 | SEMA5A | 3777303 | 24326706 | 5 | 9333338 | A | G | TTTACATTTACTGCNGCAGATATCTCTGT |
| 569 | SEMA5A | 6874451 | 23657206 | 5 | 9339718 | C | A | CTGACTGACCAGGGNTGGACAGTTAACTC |
| 570 | SEMA5A | 3822787 | 23657209 | 5 | 9346213 | A | G | ACACTGAATACCTTNTGATGGCACTGCCA |
| 571 | SEMA5A | 3734152 | 23657215 | 5 | 9352338 | T | G | TCCTGTTGGAATGGNGAATCTCATATTAG |
| 572 | SEMA5A | • | 46559487 | 5 | 9358725 | T | C | AACAAAAGTGTACGNCAAAACCCATATAA |
| 573 | SEMA5A | • | 46559486 | 5 | 9366958 | A | G | GCTTCAAGTTGGAANATAGCCTACAGTTT |
| 574 | SEMA5A | 3777325 | 24608741 | 5 | 9372778 | C | G | AATAATGTCAAGAANATGCTGGCACATAA |
| 575 | SEMA5A | 11745486 | 24135527 | 5 | 9378274 | G | T | AAAAGCAAGAGTTTNCCAAAGTGTAACAG |
| 576 | SEMA5A | 7702187 | 23657222 | 5 | 9385281 | A | T | GCAGTATGGACTGGNGCTTACATCATGTC |
| 577 | SEMA5A | 1918778 | 23657228 | 5 | 9404827 | T | C | CACATCATGAGTGTNCCCAGTTCATTGTG |
| 578 | SEMA5A | 17196614 | 24608754 | 5 | 9411947 | C | T | ACCTGTCCTCTATGNTATGACAAATATGC |
| 579 | SEMA5A | 40654 | 23657251 | 5 | 9424411 | G | A | AATAAACCATTTTCNTCAGTCTAATTATT |
| 580 | SEMA5A | 1805927 | 24608769 | 5 | 9435861 | G | A | TGCAAAGGAACCAANAAGCATGAGGTAAG |
| 581 | SEMA5A | 380206 | 23908985 | 5 | 9443722 | T | C | CTGATGAAACCTTTNAAATCCAATGGATT |
| 582 | SEMA5A | 414115 | 23909077 | 5 | 9451165 | G | A | AAATAAAATAGAGTNCAATGATATGCCCA |
| 583 | SEMA5A | 16882671 | 23278516 | 5 | 9457648 | T | C | GGCTAAATCATTTANAAACTTGTTAACAC |
| 584 | SEMA5A | 16882720 | 23909387 | 5 | 9476158 | A | G | ATTTTACTGCCCTCNGTGAGCTCAACATC |
| 585 | SEMA5A | 3846587 | 23278910 | 5 | 9482200 | C | T | TGACTTAATTCATCNGGCCATGGAGAAGG |
| 586 | SEMA5A | 17258493 | 24327355 | 5 | 9489801 | A | G | TTAGGGAGAAAAATNAATAAGTTAATAAA |
| 587 | SEMA5A | 3798028 | 23909591 | 5 | 9500717 | T | G | TGTTTCATGTTTTANAGAGGACTTAACAC |
| 588 | SEMA5A | 3798031 | 23279457 | 5 | 9510243 | C | T | GTTTGTACTAACCANGTGCTGCTTCTAAA |
| 589 | SEMA5A | 16882852 | 23279573 | 5 | 9519215 | G | A | TGAGAGTTGGGGGTNCAAGTATCTGAGTA |
| 590 | SEMA5A | 433755 | 23279691 | 5 | 9527946 | A | C | CCAACCCTCAAGGGNCATTGGTTTCTGAA |
| 591 | SEMA5A | 418768 | 23279784 | 5 | 9535178 | T | C | GACCCAAAAATTACNTTTGTCAACCCAAA |
| 592 | SEMA5A | 10513020 | 24327990 | 5 | 9541913 | G | T | AAATCCACCATAAGNTTCAAAGATTTTGG |
| 593 | SEMA5A | 17260975 | 24328064 | 5 | 9547465 | T | C | TAGGTATATGGTGANGGACATAGATCTCC |
| 594 | SEMA5A | 7735345 | 24328228 | 5 | 9570081 | T | G | TCTACCAAATCCTGNTATTCTCACATGGG |
| 595 | SEMA5A | 462057 | 23910382 | 5 | 9581021 | C | G | TACAGTGAAAAGGCNTAGAAGATCTTGAT |
| 596 | SEMA5A | 3756737 | 24328454 | 5 | 9587392 | T | C | CCCAGTCAAGATGCNTATTCTCCCCACTT |
| 597 | SEMA5A | 17262778 | 24328576 | 5 | 9594160 | T | C | TGTTTTTCTTTCCANGAGTACATCATACA |
| 598 | SEMA5A | 2115243 | 23910625 | 5 | 9599874 | A | C | AGCGGGATTAAGTCNAGGAAATGAACAAA |
| 599 | SEMA5A | 17263917 | 24328907 | 5 | 9605338 | G | A | AGATGAGATTTGAANGATGAGTAGAGACC |
| 600 | RASA1 | 6874962 | 24145154 | 5 | 86595943 | T | G | ATTATACCACATATNGTAATCTCTGCAGA |
| 601 | RASA1 | 7713115 | 23674870 | 5 | 86607880 | T | G | GAATACGGATGTTGNTTATTGTTTGAATA |
| 602 | RASA1 | 12659702 | 24145156 | 5 | 86634924 | T | A | CATTAATTGTGTCCNATTTTTTTTTTCTG |
| 603 | RASA1 | • | 46558070 | 5 | 86641699 | A | G | TTGTTGCTTTTGAANATCAATTGACTGCT |
| 604 | RASA1 | 16902623 | 23674873 | 5 | 86652405 | T | C | TGACAAGCAAAAATNGAATGATGACTGCA |
| 605 | RASA1 | • | 46558935 | 5 | 86685599 | T | C | TTAAGGGTTTTGGGNACCCAGCTGCAATT |
| 606 | RASA1 | • | 46558934 | 5 | 86706726 | T | C | TAAGATTAAAATATNTAGCCACCCTCCTT |
| 607 | CCNH_RASA1 | 1062035 | 24621570 | 5 | 86718861 | C | G | GACACAGTTCTGACNAGGTTAAATAAGTA |
| 608 | EFNA5 | 365807 | 23919166 | 5 | 106741517 | A | G | ATATCGATCAAAAANGTCATGTTGTTCCA |
| 609 | EFNA5 | 3797510 | 23919211 | 5 | 106753230 | C | G | TCTTGGATTTAGTANAGGTCTTTCAGATT |
| 610 | EFNA5 | 252813 | 24339833 | 5 | 106761925 | G | A | TAACCCTCTTCCAGNTTCCCACAGAGATC |
| 611 | EFNA5 | 682609 | 24656199 | 5 | 106770052 | T | C | TCCACCTGGAAAACNCTCTTGCCAAATAG |
| 612 | EFNA5 | 468998 | 23940994 | 5 | 106776173 | T | C | AGAACATTTCTCCANGTTTCTCAGGTATG |
| 613 | EFNA5 | • | 46558782 | 5 | 106783044 | A | C | CCGATATAATTCTTNAAAAATATGCTGGC |
| 614 | EFNA5 | 152609 | 24172380 | 5 | 106798537 | T | C | ATTACATTCTGAAANTGAAATGTAAAAGA |
| 615 | EFNA5 | 152591 | 24172420 | 5 | 106811193 | G | A | AATGTAAGAGAAACNTCTACCAAATGCTG |
| 616 | EFNA5 | 17533588 | 24656328 | 5 | 106817663 | A | G | ATGAACAAAAGCCANTAATTCCCTTCATA |
| 617 | EFNA5 | 17449339 | 24656382 | 5 | 106830433 | G | A | CTCCAATGTTGTGGNAAGTGTGATAGCTG |
| 618 | EFNA5 | • | 46558780 | 5 | 106836832 | C | G | TTGACTCTAAAAAGNCACATCATGCCCCA |
| 619 | EFNA5 | • | 46558779 | 5 | 106844352 | G | A | CTCTTGCAGTCAGCNGTCCCAGAAAGACA |
| 620 | EFNA5 | 26239 | 23723814 | 5 | 106850166 | G | A | TTCTCCTTCTGGGCNTATGAGAGTAGTAT |
| 621 | EFNA5 | 153695 | 24172538 | 5 | 106856995 | G | C | TGCTGAATGTGTGCNGTAACTGCAGTGTT |
| 622 | EFNA5 | 25976 | 24656482 | 5 | 106862909 | G | A | CTTTCTGTCTCTGANTAACAACAGGTTGC |
| 623 | EFNA5 | 152562 | 24656498 | 5 | 106869682 | C | T | CTCTTGAGCACTTANGCTTTATTTGTGTA |
| 624 | EFNA5 | 251960 | 23723928 | 5 | 106876063 | C | A | CCTGTTTTTTTTTANCTTCTAGGAGGTTT |
| 625 | EFNA5 | 250253 | 24656534 | 5 | 106880865 | T | C | TCTCACAGTATTCANCATGCAAATTATGG |
| 626 | EFNA5 | 446774 | 24339874 | 5 | 106889695 | A | G | TAGCCAGCAAATACNGAATTACAGAAATC |
| 627 | EFNA5 | 341276 | 23296947 | 5 | 106898137 | U | C | AGCACAATTTTTCCNTTTTCACATTTCAG |
| 628 | EFNA5 | 163893 | 23297022 | 5 | 106905473 | T | C | TCTCTCTGTTCTACNATCTTTAAACATCT |
| 629 | EFNA5 | 17413725 | 24340203 | 5 | 106915238 | C | G | ACTTTAGCAACTCCNTGCCGATCTCCATA |
| 630 | EFNA5 | 2484103 | 23919539 | 5 | 106924516 | T | C | CTATAGTTCTGTAANGTGAGCGCCACCTT |
| 631 | EFNA5 | 3844311 | 23297774 | 5 | 106953865 | T | C | CCTATATTCTAAACNCTACCTTCCAAACA |
| 632 | EFNA5 | 26732 | 24340647 | 5 | 106967264 | A | T | TCCCATGCTTTGGTANAGTACGTTTACTGA |
| 633 | EFNA5 | 26727 | 23919880 | 5 | 106973590 | C | G | AATGAGACAGATAGNAATGTTCAATAGAA |
| 634 | EFNA5 | 164842 | 23920014 | 5 | 106990603 | G | A | GTAAGATTTTCTGTNTAGTATCATCAAAT |
| 635 | EFNA5 | 17160287 | 23920034 | 5 | 106999002 | A | C | AACCACCACGAATGNAGGAAATCTTCCCC |
| 636 | EFNA5 | 17160302 | 23920070 | 5 | 107007644 | T | C | AATTAGAGTAGCTCNCAGTATCTGTCTCT |
| 637 | EFNA5 | 165263 | 23920109 | 5 | 107017374 | T | C | TCCAACACTGATTANTGCAGTCTTAAAAC |
| 638 | EFNA5 | 13170272 | 23920171 | 5 | 107031948 | G | A | CTGCAGGCAGGGCANATCTATTAAAAAAC |
| 639 | EFNA5 | 12188264 | 23920194 | 5 | 107037963 | T | C | GCCCCATTTTAGGGNGTGTGTTTTTTCTG |
| 640 | SEMA6A | 33066 | 23444522 | 5 | 115800733 | T | A | AGGATGGGAACTCANAGTCATTTAAGTTG |
| 641 | SEMA6A | 7728334 | 24453482 | 5 | 115818214 | G | A | CCCAGCCAGTCGAGNTGACCACGCTGAAA |
| 642 | SEMA6A | 1046683 | 24453505 | 5 | 115832928 | A | G | TTTCTGAGGTTCACNTCTCATGTCATGAT |
| 643 | SEMA6A | 3213797 | 24009047 | 5 | 115839418 | A | G | TAAGCAGTGAATTCNGATATTATTCAGGG |
| 644 | SEMA6A | 17139898 | 24009156 | 5 | 115845741 | C | T | GAACTTGTACAATANCCTGTACCTCTCCA |
| 645 | SEMA6A | 9885339 | 24009255 | 5 | 115853167 | C | A | CAGGCTTGACAGTTNACTAAACCCTCGCA |
| 646 | SEMA6A | 2287686 | 24453945 | 5 | 115859229 | A | G | TGAGGACTTCTCCCNTTAAAAGTTCAAGT |
| 647 | SEMA6A | 54100 | 24454057 | 5 | 115866016 | C | G | GGTCACAAAAGAAANCCGTGTTTAAGTCA |
| 648 | SEMA6A | 153600 | 23445520 | 5 | 115872595 | A | G | GTCTATACACACAANTTAATGAACACTGC |
| 649 | SEMA6A | 1553530 | 23445738 | 5 | 115882495 | C | G | GAGGAGATTCCCCANGTTCACTTTGAATA |
| 650 | SEMA6A | 153645 | 23445853 | 5 | 115892477 | G | A | ACTTTAATTTCGCCNTCCCCTTCCAGCAG |
| 651 | SEMA6A | 254224 | 24454559 | 5 | 115909567 | T | A | GTCAAGGGACACAGNAGAGCTATTTCAGG |
| 652 | SEMA6A | 254234 | 23446179 | 5 | 115916094 | T | C | GGTCAACTGATGATNTGCTAATAAATGAA |
| 653 | SEMA6A | 254208 | 23446273 | 5 | 115922794 | A | C | TAAACATACTCAAANATTTCAGCAGTCTT |
| 654 | SEMA6A | 2840113 | 24009926 | 5 | 115931808 | T | C | TTCCACAATGCGTGNAATA7TTCACTAAA |
| 655 | ABLIM3 | • | 46558439 | 5 | 148494322 | C | A | CACTGGCCCCTGGANACGTTGTCCTCACA |
| 656 | ABLIM3 | 6580598 | 24195973 | 5 | 148513945 | A | G | AGCAGGGGCAGCCCNAGGTCTATTCCTAT |
| 657 | ABLIM3 | 9763202 | 24683764 | 5 | 148528612 | G | A | TCTAGTCTAGCCCANGATTTCTTGACCTT |
| 658 | ABLIM3 | 17109719 | 23763725 | 5 | 148539907 | A | G | AGGCAGTTTCTAACNTAAGAGGTAGTTCA |
| 659 | ABLIM3 | 17795663 | 24196025 | 5 | 148555253 | G | C | CCTTTCTACCAAAANCTTCCTCCTTCAGC |
| 660 | ABLIM3 | 2918286 | 24196045 | 5 | 148564824 | C | T | TGATCACTGGATGANCATGTCAAGCAAGA |
| 661 | ABLIM3 | 2918280 | 24196054 | 5 | 148570813 | A | T | GCTGGGAAAATCAGNTGTATCTTTGATTC |
| 662 | ABLIM3 | 2963498 | 23763834 | 5 | 148578325 | T | C | CTGTGTGGCCAGGGNGCAGACAGAAGTGA |
| 663 | ABLIM3 | • | 46558437 | 5 | 148586949 | G | A | GTGTGCTATAACCANTCAGAGAGATAAGA |
| 664 | ABLIM3 | 10079296 | 24683904 | 5 | 148593382 | C | T | CTCATTTCCCACTANTTAACTGAGAGCAA |
| 665 | ABLIM3 | 3816159 | 24683926 | 5 | 148605091 | G | A | TGAAGATGACAAAANTTTCTCAATGCAGC |
| 666 | ABLIM3 | 17709363 | 24683942 | 5 | 148613377 | G | T | GGAAGGATGTAGACNGACTAGAAAGACAA |
| 667 | ABLIM3 | 1866376 | 23763933 | 5 | 148620386 | G | C | TCTGTAGAATGCAGNATCCCTCAAGATGG |
| 668 | ABLIM3_FLJ3 | 10515625 | 24196157 | 5 | 148627746 | T | C | GTTTTTCTTACTCTNCCTGAAGTCTCCTG |
| | 6748 | | | | | | | |
| 669 | SLIT3 | 10516048 | 24185906 | 5 | 168026895 | C | T | AACTGAGGTAGATGNTATAAAGAAAGGGA |
| 670 | SLIT3 | 1870563 | 23747144 | 5 | 168036018 | T | G | AGGAATCTGGTTTTNTGATGGGGAGCCAA |
| 671 | SLIT3 | • | 46558313 | 5 | 168041434 | C | T | CCTTGACATGTCTGNGTAAAGGCTTAATC |
| 672 | SLIT3 | 17553085 | 24671664 | 5 | 168050721 | C | T | CAAATCTTCTTCCCNTAGGTCCATGGCAA |
| 673 | SLIT3 | 17553140 | 24671680 | 5 | 168059653 | A | G | TGGGGATAAATTGTNCTTCCCTTACAGGG |
| 674 | SLIT3 | 11746577 | 24185993 | 5 | 168066734 | T | C | CTTAATCACAGCTCNACTGCTTACAAGCT |
| 675 | SLIT3 | 4868530 | 24671699 | 5 | 168072724 | T | C | GAGCCGAACACTCANTGTTATGGAAACGG |
| 676 | SLIT3 | 7715777 | 24186011 | 5 | 168079907 | T | C | GGGACAAATGATGCNGAGGAAAGAGAAAA |
| 677 | SLIT3 | 17665285 | 24186015 | 5 | 168084887 | G | A | CTTAAGATAAGAACNTCTCATTGTAATTA |
| 678 | SLIT3 | 7728435 | 24671722 | 5 | 168099959 | T | C | GCTGGGGTCTTGCANGCTTGCAACAGCCT |
| 679 | SLIT3 | 10036727 | 24671734 | 5 | 168112659 | C | T | ATTACTCACACAGCNGATCAAGTTACTCC |
| 680 | SLIT3 | 2288795 | 24671756 | 5 | 168122403 | C | T | TCAAGTAACAGACANGGCATCGCCTTTGC |
| 681 | SLIT3 | 4867902 | 23747338 | 5 | 168132202 | C | T | AATCCATAAGCTAANTGGTTTTCTGCAGG |
| 682 | SLIT3 | 7720465 | 23747376 | 5 | 168142029 | G | A | TGCTTTGGGAGGAGNGACCTCTGAGCTAC |
| 683 | SLIT3 | 891926 | 23247169 | 5 | 168149539 | C | T | AAATTAGGGCCTTANGTTTCATTACCAAG |
| 684 | SLIT3 | 17665720 | 24186159 | 5 | 168157173 | A | G | GAGACTAATCAAGGNACATGATGTGACGC |
| 685 | SLIT3 | 17733268 | 24186173 | 5 | 168164017 | T | C | AGTAAATTAGATAGNATGTTGGAAGGCCG |
| 686 | SLIT3 | 905799 | 24671909 | 5 | 168177121 | A | G | GGCCAGACAGAGTGNCCAAAACCCCTGTT |
| 687 | SLIT3 | 924875 | 24186242 | 5 | 168182928 | C | G | ACTGTGGAGATAGANAAGGGAGATGGTCA |
| 688 | SLIT3 | 2938759 | 24671970 | 5 | 168196154 | G | A | GAACTATTTTTCCCNTTTGTTCTAGGTGG |
| 689 | SLIT3 | 12523259 | 24186327 | 5 | 168210546 | A | G | ATACTTTCCTGTATNTTGGGCTTTTGGCA |
| 690 | SLIT3 | 7736650 | 24186339 | 5 | 168219168 | T | C | TGGTTTTCAAGCAANGTGGTTTTCAATTT |
| 691 | SLIT3 | 2974437 | 24186344 | 5 | 168227746 | T | A | CAACTGTAAGATAANCTCTGTGTGCATCA |
| 692 | SLIT3 | • | 46558310 | 5 | 168234495 | C | T | ATATGCTTGCTGTGNATGTCTCCCCCTGT |
| 693 | SLIT3 | • | 46558309 | 5 | 168240947 | C | T | ACTATTAAGAACAANTGTGGGATTTATTC |
| 694 | SLIT3 | • | 46557969 | 5 | 168267877 | A | G | TGCTTAGCAGTGGTNGACGAGTCACCTGG |
| 695 | SLIT3 | • | 46558308 | 5 | 168274485 | T | C | TAAAAATGTAGGCTNACTGTCACTAGCCA |
| 696 | SLIT3 | 1982062 | 24186388 | 5 | 168288225 | C | T | AAAACCTTTAAAAANATATCTCCAGTGTT |
| 697 | SLIT3 | 13160953 | 24186405 | 5 | 168300611 | C | T | TGGACAATGAAAACNTAGTCAATTTTAAC |
| 698 | SLIT3 | 7712757 | 24186423 | 5 | 168306966 | A | G | CTGCAGGGAGAGCCNCCGTCTGCCGCCCT |
| 699 | SLIT3 | 6555838 | 23747738 | 5 | 168314072 | G | A | CCTGTCAGATTCCCNGCTCACAGCTCAAA |
| 700 | SLIT3 | 6885618 | 23747753 | 5 | 168324520 | A | G | ATGTGTCCAAAAGCNGTCTGCAGGTCACA |
| 701 | SLIT3 | 7708885 | 24186462 | 5 | 168328507 | C | T | CTGTGCACTGATGANTTCAGAAGCAGAAG |
| 702 | SLIT3 | 13359468 | 24186557 | 5 | 168376646 | A | T | TCTAGTATTCTTGANAGCAACTGCTTTAA |
| 703 | SLIT3 | 1432899 | 24186575 | 5 | 168391547 | G | A | CAAGACTTAGAAGANTAAGCATGGTAGAA |
| 704 | SLIT3 | 7717558 | 24186608 | 5 | 168402556 | G | T | AAAGGGTGTTTACTNGCAACATTTTTGAA |
| 705 | SLIT3 | 11134555 | 24186628 | 5 | 168410507 | C | A | TTAGATTCAGCAGCNTTCCAGCGCTCTGC |
| 706 | SLIT3 | 10516058 | 24672317 | 5 | 168431265 | T | C | TTACTGTGTGCAAGNGTAGAATCCAAGTC |
| 707 | SLIT3 | 17636543 | 24672339 | 5 | 168442310 | T | C | GAAGAGTAGAAAAANAGTAGATGAAGATG |
| 708 | SLIT3 | 296005 | 24672357 | 5 | 168449379 | A | T | TGTAACCATAGATTNACTTGAGTGATAAG |
| 709 | SLIT3 | 10067964 | 23748147 | 5 | 168458268 | C | T | AATGTGTCAAATGANAGGAGGGTTAAGTG |
| 710 | SLIT3 | 295996 | 23748190 | 5 | 168465045 | A | G | AATGCAGACACGCCNTGATGACTGTGGTG |
| 711 | SLIT3 | 2668053 | 24186720 | 5 | 168481279 | T | A | GGACAATCAACACTNCTTCCTGGTTTGAA |
| 712 | SLIT3 | 9688032 | 24186737 | 5 | 168488773 | T | A | AGAGCACGTGTGAGNAAGATGCCCCTCTC |
| 713 | SLIT3 | 17735037 | 24186764 | 5 | 168496065 | G | A | ACTTTGCTGTTACCNCTCCTATGACTGTG |
| 714 | SLIT3 | 2161115 | 24672472 | 5 | 168503474 | G | A | AGAGCACTCACAATNGGCAGAGTCACATG |
| 715 | SLIT3 | 297811 | 24672543 | 5 | 168516070 | C | A | CTAGGATCTGCAGANGTATTCCCTGGCTG |
| 716 | SLIT3 | 10516060 | 14916782 | 5 | 168522506 | C | T | GTAGGGAGATAGCTNTAAGGGAGAAAGAC |
| 717 | SLIT3 | 10462980 | 24672584 | 5 | 168529461 | T | G | AGAGAAACTTCAAGNCCTGATCTTGACTG |
| 718 | SLIT3 | 297869 | 24672601 | 5 | 168534849 | A | G | CTATATCCATTTACNCAGTGAATACTGAG |
| 719 | SLIT3 | 6869130 | 23748515 | 5 | 168541859 | G | C | GCTCACCAAATTTANGCAAGGAGCATGAA |
| 720 | SLIT3 | • | 46558303 | 5 | 168548441 | G | C | GTAACTTGCCAAAGNTTTCAGGATACACA |
| 721 | SLIT3 | • | 46558302 | 5 | 168554351 | A | G | GGATGTTGGCCAACNGAGCCAGTTTCTCT |
| 722 | SLIT3 | 17070983 | 23748607 | 5 | 168560914 | T | A | ACCCTCACTGAAGANGTTTGCAAGAACCC |
| 723 | SLIT3 | 11738097 | 24186900 | 5 | 168566025 | 1 | G | CACAGGTAGGAAACNGCCAGATGCAAATT |
| 724 | SLIT3 | 17070994 | 23748656 | 5 | 168575990 | C | G | GAAGTTCTAATACCNACCTGACACGTTTC |
| 725 | SLIT3 | 17735629 | 24186957 | 5 | 168586080 | T | C | GCAGTCTCTGATCTNCGGGACTAAAAACA |
| 726 | SLIT3 | 17668151 | 24186978 | 5 | 168593330 | T | A | TTCCAATCTTTATANCCATTCTTTTTCCT |
| 727 | SLIT3 | 4868461 | 24305960 | 5 | 168625096 | C | G | GCTTAATCTTTTTTNGCATCAGAGTTTGA |
| 728 | SLIT3 | 7729641 | 24306108 | 5 | 168638962 | C | G | GCATGGAAGAGCAGNACCTTATGTTAGGC |
| 729 | SLIT3 | 2338526 | 24186992 | 5 | 168657826 | C | T | GAAGAGACCAGAGANATCAAAGAAGCTCC |
| 730 | HK3_UNC5A | 539822 | 23337829 | 5 | 176242770 | A | G | CTTGCTGAGTGGTCNGTTCTGAGAAGGCA |
| 731 | EPHA7 | 1486757 | 24012014 | 6 | 93998582 | C | T | GTAACAATTTTACANGTCTACTTTTGAAG |
| 732 | EPHA7 | 9354002 | 24012053 | 6 | 94004028 | T | C | GGACTCCTTTATTANGAGAGAAGCCATCA |
| 733 | EPHA7 | 431650 | 24457585 | 6 | 94010599 | T | C | GTGTTTTGCCTTTCNTAATAGCCAGATAC |
| 734 | EPHA7 | • | 46560452 | 6 | 94021040 | C | T | CCAGTAATACTGGCNATTAATGTATTCAA |
| 735 | EPHA7 | 345726 | 24457789 | 6 | 94029125 | T | G | TCTTATTCAATTTGNATTTGTGTTACATA |
| 736 | EPHA7 | 345717 | 24457872 | 6 | 94041750 | A | G | AACAATAAAGTTCCNAATTGAAAGATGTA |
| 737 | EPHA7 | 346462 | 24012341 | 6 | 94061206 | T | C | AATGCATACATTAANAGATATAGATTCAC |
| 738 | EPHA7 | 1684822 | 24458061 | 6 | 94074770 | T | A | TAGAATAGATTACANTATACCAAAGGAAA |
| 739 | EPHA7 | 164294 | 24458118 | 6 | 94082305 | A | G | ATCCTCTCTTTAGGNATCCACAGTTTATG |
| 740 | EPHA7 | 9354004 | 24012485 | 6 | 94089228 | C | T | TGAGGGAAGCCTTCNAAAAGTCACCTACA |
| 741 | EPHA7 | 164560 | 23362319 | 6 | 94098867 | T | C | ATGAGCTTTGTATANTTGTAATCTTTTAT |
| 742 | EPHA7 | 4706437 | 23958022 | 6 | 94107893 | G | A | CATTAATCAGTTTCNGAAATTAAGAGATA |
| 743 | EPHA7 | 164536 | 23362565 | 6 | 94115082 | T | C | TACAGTTTTAGGACNAAATTGCAAAATGA |
| 744 | EPHA7 | 10944667 | 23958242 | 6 | 94126000 | T | C | TAATATTCTCCACTNGGGTATTATGCAAA |
| 745 | EPHA7 | 4707796 | 23958267 | 6 | 94133139 | T | C | TCTGCCAACCGGTTNACCTCTCGCTTTCA |
| 746 | EPHA7 | 1895639 | 23362934 | 6 | 94141033 | G | C | TATTTGCCAAAATANAAGAAACTTGTATC |
| 747 | EPHA7 | 621890 | 23363021 | 6 | 94151853 | G | T | TACTTTTAACTCCCNCAAAACTTGTTTTC |
| 748 | EPHA7 | 665252 | 24390428 | 6 | 94159178 | C | A | TTTATTTCATATTGNAAAATGTATGCATG |
| 749 | EPHA7 | 376102 | 23958503 | 6 | 94169700 | A | C | ATCTTTGCTTTGCCNGTGGATAGTTTATG |
| 750 | EPHA7 | 351332 | 23958634 | 6 | 94186765 | T | C | CCTCCGCCCTGTCTNTTCACCATAGTTTG |
| 751 | EPHA7 | 578847 | 23363512 | 6 | 94193761 | C | T | GCTACTTCCATCACNTACAGATCCAAGCA |
| 752 | FYN | 1794834 | 24682177 | 6 | 112081702 | G | A | ACAAATTATGCTTTNGTGAATAGTCCTAG |
| 753 | FYN | 9387025 | 24194538 | 6 | 112091093 | G | A | TGGAATCCTCCCCANTATCTGACCCGGTT |
| 754 | FYN | 2237255 | 24194543 | 6 | 112099520 | C | T | AATGGAAATCATGANGGACCGGGACTGGG |
| 755 | FYN | 2344706 | 24194548 | 6 | 112105713 | A | T | TGCAGTTAACTTGGNATTGTTATGTAACT |
| 756 | FYN | 2237258 | 24194560 | 6 | 112112848 | C | T | TATCCTTCCAGTTTNCGAAGAGCCAGAAA |
| 757 | FYN | 809193 | 24194567 | 6 | 112127107 | T | C | CATTGAGCAAAACGNCCATGGTCTTTCTT |
| 758 | FYN | 9487709 | 24194574 | 6 | 112133093 | T | C | AAAGTCAGATTAGANAGATATGATATGAA |
| 759 | FYN | 804184 | 24194588 | 6 | 112141801 | T | C | AGCAGTTTATAAAANAAAGAACTTGTTGC |
| 760 | FYN | 10484480 | 24194607 | 6 | 112153017 | T | C | TTCAGGATAGAAGANTCATGGCAGAGGGC |
| 761 | FYN | 697642 | 23761260 | 6 | 112159798 | G | A | TAAAGTTTTCACTANAGGTCTGCATTTAT |
| 762 | FYN | 6568703 | 23966269 | 6 | 112172394 | T | A | CTCAGCTGGAAAGANCTGTTTTTGCTGGA |
| 763 | FYN | 6910116 | 24400760 | 6 | 112182621 | A | G | TGGTTTTGGCCTTANTATACAGTAGTTTC |
| 764 | FYN | 13218316 | 24400795 | 6 | 112189727 | C | T | TTGCCCAGGGTCTCNTTGTACTTTTAGCA |
| 765 | FYN | 9372311 | 24400837 | 6 | 112196361 | G | A | AAGCTTATTGAGGTNTAATTTGTATACAG |
| 766 | FYN | 12910 | 24400874 | 6 | 112208468 | G | T | ACACTTACCAAAATNTCCGCCAACGATCA |
| 767 | FYN | 1022649 | 23376623 | 6 | 112226674 | A | G | GGTGAAGAATAAACNAAAAGCGTTCTGGG |
| 768 | FYN | 9481192 | 23376736 | 6 | 112237171 | C | T | ATCCACACAACTAANGTCAGTTCCACTAA |
| 769 | FYN | 9387040 | 23376755 | 6 | 112238271 | G | A | GAATTAAATTACAANTGCAATCAGTCATG |
| 770 | FYN | 2018548 | 23248694 | 6 | 112263263 | C | T | TGATCACTGAAATANTAAAGTGATACTAA |
| 771 | FYN | 9487735 | 233768606 | 6 | 12272269 | T | C | GCCAACAGGTATCANTTACTTATACAACA |
| 772 | FYN | 910683 | 23966677 | 6 | 112289980 | C | T | ATAATACTTTCAGGNCCAACAATACTGGC |
| 773 | FYN | 9320379 | 23376984 | 6 | 112291314 | T | C | CTTCTAAATAGACANGACATTTGTGTGGA |
| 774 | RAC1 | • | 46545898 | 7 | 6183561 | A | G | TGGAGTTCTAGTGGNATGTTGGGTAGAAG |
| 775 | RAC1 | 836483 | 24507116 | 7 | 6200981 | A | G | ATTGCTTACTCCCTNTAGGCAGCAGAATT |
| 776 | RAC1 | 836475 | 23520220 | 7 | 6207172 | G | A | CGCCATTCTTAGTTNAAATTGCCCTTCAG |
| 777 | LOC221955_R | 836548 | 24052532 | 7 | 6212688 | A | T | TACTTGATTTTTTCNTTTTAGATAGTTAG |
| | AC1 | | | | | | | |
| 778 | LOC221955_R | 836559 | 24507263 | 7 | 6222003 | G | C | CACACAAGCACTATNGTGACGCGCGCTTT |
| | AC1 | | | | | | | |
| 779 | LIMK1 | • | 46545406 | 7 | 72949010 | A | G | AGGGGTGAGTCCCCNGGTCAGGGCAGTAA |
| 780 | LIMK1 | • | 46545405 | 7 | 72949839 | C | T | GCAAATAATTATGGNGTGCCCACTCTGCA |
| 781 | GNAI1 | 2523189 | 24511553 | 7 | 79421079 | C | T | TTCTTTTGATGATANGATTTGTTCAGGTCA |
| 782 | GNAI1 | 2714467 | 23526850 | 7 | 79428777 | G | C | TTTGATTATTGTCANTGGAGCTTTGGGCT |
| 783 | GNAI1 | 2714449 | 24511673 | 7 | 79436598 | G | A | ACATTTCAGTACCANTGGAAAATCTCTCC |
| 784 | GNAI1 | 916905 | 23527045 | 7 | 79444998 | C | T | TTACAGTTGAGGTTNATTACTTCTGGTTC |
| 785 | GNAI1 | 6980462 | 23532970 | 7 | 79453486 | C | G | AAAGACATGTAGTTNAGGTATAAAGATGC |
| 786 | GNAI1 | 17153558 | 23533094 | 7 | 79470150 | A | G | TCTCAGAGCACATGNATATATGAACTGGA |
| 787 | GNAI1 | 10256687 | 24515673 | 7 | 79488138 | G | A | ATAGCTCTTTTTGTNTACGGTGAAATTAC |
| 788 | GNAI1 | 6954752 | 23533479 | 7 | 79494577 | A | C | TCTTCATTTCAGTANCCTTCTAAAAGTCT |
| 789 | GNAI1 | 2886609 | 23533530 | 7 | 79501062 | T | C | TCACCAAGTCAATTNAATACTTTTAATTT |
| 790 | SEMA3C | • | 46545359 | 7 | 80011267 | C | G | ATTCCTGAGGGGGANTCGGATGATGAAG |
| 791 | SEMA3C | • | 46544632 | 7 | 80019837 | A | C | TTTTCAATTATCCCNAAATAAACAATGTC |
| 792 | SEMA3C | • | 46544524 | 7 | 80038069 | C | T | ACCCTACCCACTTANGTGCTTATAAAGTC |
| 793 | SEMA3C | 12669309 | 24061990 | 7 | 80044821 | C | A | AAATCAGGAGCATTNAAGCTTGTAAATCT |
| 794 | SEMA3C | 3778677 | 24062025 | 7 | 80055755 | C | T | TACATTCCTATCACNAGAGGGTTAAATAG |
| 795 | SEMA3C | 3801355 | 23537866 | 7 | 80066070 | T | C | TAACTAGACTTTCANCTGGAGTTCATATG |
| 796 | SEMA3C | 12673477 | 24062109 | 7 | 80075398 | G | T | ATGTTTAAAGTGTANTAAGGCAAAAACAA |
| 797 | SEMA3C | 17154487 | 24062179 | 7 | 80086563 | C | G | TGCTCTCAACTGACNTACTCCTACATTGC |
| 798 | SEMA3C | 1527481 | 24062270 | 7 | 80096211 | C | T | ATTTTTAAATGTACNGTAGAAGGAAAGAA |
| 799 | SEMA3C | 7341415 | 24422292 | 7 | 80114614 | G | A | AAGTGGAGGTACACNAGCAACTGCAGTGT |
| 800 | SEMA3C | 11974793 | 23404227 | 7 | 80134633 | A | G | GATCCCAAACTGCCNAGGTCAAGGGCCCA |
| 801 | SEMA3C | 2189958 | 23404307 | 7 | 80149388 | A | G | TTACTTAAAGACACNTTTCTGGGAGAAAT |
| 802 | SEMA3C | 17154586 | 23404432 | 7 | 80168572 | T | C | ACTATGACTACACANTATTACTTTTTCTT |
| 803 | SEMA3C | 12707079 | 24422706 | 7 | 80189182 | C | T | TAGTCTGTAAGATGNTTTAGTAATTAAGG |
| 804 | SEMA3C | 12707081 | 23404657 | 7 | 80191698 | T | C | AGAAACACAATCATNTTGCTTCTACCATA |
| 805 | SEMA3E | 17212207 | 24521210 | 7 | 82633963 | C | T | ACTACCATTTGTACNTGTGTGGGCGTAAA |
| 806 | SEMA3E | 2723005 | 23541202 | 7 | 82646206 | T | C | GAGCACTTAGTAACNGATGAAAGAGGGCT |
| 807 | SEMA3E | 10273103 | 23541299 | 7 | 82654593 | A | G | GTTATTATGCCACCNCCCTACCCAATCTC |
| 808 | SEMA3E | 17213034 | 24521444 | 7 | 82660426 | C | T | AGATGAGATTTCCTNCATTCCTAACCCAT |
| 809 | SEMA3E | 2722974 | 24064408 | 7 | 82674089 | C | T | CCATCTGTTTTTACNAATATTGGTTTTTT |
| 810 | SEMA3E | 2709962 | 24521790 | 7 | 82685866 | T | C | TGGAGATGGGCGAGNTTAACAACTTCTCA |
| 811 | SEMA3E | 2713141 | 24521985 | 7 | 82697166 | C | T | AAATCTGTGCAATANGTAAATCTACCTTA |
| 812 | SEMA3E | 2709948 | 23542097 | 7 | 82707789 | G | C | GGTGTAGCTCAAAANTTTTTATAGCTCTA |
| 813 | SEMA3E | 2713174 | 24522178 | 7 | 82720608 | C | T | ACAATTGTTACCAANGTTAAACTCTCTAC |
| 814 | SEMA3E | 2466440 | 24522243 | 7 | 82732623 | T | C | TTTCAAGTAAACTANGCTGTGTATGGTAC |
| 815 | SEMA3E | • | 46545341 | 7 | 82742338 | A | G | ACATCTGAGAGGGANCCTATTAGACCACA |
| 816 | SEMA3E | • | 46544640 | 7 | 82751519 | G | A | CTCAAGCAGTTTATNTGCCAGAATTATAA |
| 817 | SEMA3E | 3801511 | 24065198 | 7 | 82764555 | T | C | CCTCTAATGTATAANGGTTCCAAATCTAC |
| 818 | SEMA3E | 2535378 | 24353491 | 7 | 82771456 | T | G | AAATTGAATTTCAGNCAGTAAGCATTGTT |
| 819 | SEMA3E | 17504676 | 24353553 | 7 | 82777248 | A | G | TCTTCTTTTTATCCNTTCCCATCTCAGAT |
| 820 | SEMA3E | 7796415 | 23315579 | 7 | 82784730 | C | G | AACTGCCCTAAAAGNTACAAAGCAAATGT |
| 821 | SEMA3E | 17445947 | 24353750 | 7 | 82796965 | A | C | GCTGTAGCATTTAANCAACTATGCACAGA |
| 822 | SEMA3E | 17157750 | 23316288 | 7 | 82809710 | C | G | GTGGATTTTGTTAANAAGAGGTTAGAAAA |
| 823 | SEMA3E | 17447180 | 24354096 | 7 | 82816332 | C | T | AATAAGGTCAAACTNTTGATAACTAGAAT |
| 824 | SEMA3E | 1639305 | 23930502 | 7 | 82822751 | C | T | CCTCTTAGGTATGANTGCATGCCTGAAGC |
| 825 | SEMA3E | 17157824 | 23930644 | 7 | 82834737 | C | T | GTGTGATCTAGGTGNCAATGAACTTTGCC |
| 826 | SEMA3E | 6951661 | 23317012 | 7 | 82840439 | G | T | TATTTTAACTCTTGNCCCCATTATCATAT |
| 827 | SEMA3E | 215285 | 24354535 | 7 | 82848050 | T | A | ATCTTCAAATACTANCCTTGACTTTTTCT |
| 828 | SEMA3E | 17157869 | 23930818 | 7 | 82855057 | A | G | CTGTCAAAAGTAACNTCAGAACACAAAAG |
| 829 | SEMA3E | 215313 | 24354796 | 7 | 82865983 | G | T | AAGTGTAATGTTTTNTTCCAATTGTGAGA |
| 830 | SEMA3E | 10239110 | 23930978 | 7 | 82872339 | A | T | AATGCCACCATGATNAATGATGCCATTTT |
| 831 | SEMA3E | • | 46545337 | 7 | 82882500 | C | T | ATAATTCTGAGCTTNACATTTTTCATACT |
| 832 | SEMA3E | 42002 | 23931162 | 7 | 82905736 | C | T | GGTTCAACAGAGTCNATGAGAAAATCCTT |
| 833 | SEMA3E | 3757623 | 23317976 | 7 | 82914666 | A | G | TACACAGAAGGCCANTGAATACATATCTT |
| 834 | SEMA3E | 13223636 | 23931308 | 7 | 82930809 | G | A | ATCATGTTTCCAAANAGAAATTAGTGTAT |
| 835 | SEMA3A | • | 46545340 | 7 | 83232566 | C | T | AGGATGATGTTGAANACATTGAAATGGAG |
| 836 | SEMA3A | 797807 | 23985992 | 7 | 83245549 | A | T | CAATGTCTAGTATGNCATTTTCACCAATT |
| 837 | SEMA3A | 6943412 | 24425461 | 7 | 83252822 | G | A | TTTTTAAAGACAGCNTGTCACTCATGAAA |
| 838 | SEMA3A | 10499878 | 23544253 | 7 | 83284338 | A | G | TTCTCCAAACATACNCATTTCTAGTTTAG |
| 839 | SEMA3A | 17158507 | 24066332 | 7 | 83287362 | C | A | AATAAGTGAAATACNTCAGATAGTTAAAG |
| 840 | SEMA3A | 10235789 | 24523550 | 7 | 83301451 | T | C | ACTAGTTTATTCTTNAGAGCAATACAAAT |
| 841 | SEMA3A | 1524879 | 24066413 | 7 | 83310121 | A | C | CAATGGAATCCAAANATTTGTTTAATCTA |
| 842 | SEMA3A | • | 46545339 | 7 | 83318974 | T | C | GGAAGAGATGCTGANTGAGGTAATTTGAG |
| 843 | SEMA3A | 2527031 | 24422869 | 7 | 83342828 | T | C | AAAACTATCCTTCGNCATGGTGTCAGGTA |
| 844 | SEMA3A | • | 46545338 | 7 | 83354478 | C | A | ATTTTGAGTAGTTGNAATTTTGATTAATA |
| 845 | SEMA3A | • | 46545336 | 7 | 83362088 | C | T | TTTTCATTATAAATNCACATGCTGTCATC |
| 846 | SEMA3A | • | 46545335 | 7 | 83367998 | A | G | AATGTCTGTACACANAGGCTACCAGGCTA |
| 847 | SEMA3A | 17232537 | 24423517 | 7 | 83373994 | T | C | AGCAGTAAACTCTANGCACACTGGGCTAT |
| 848 | SEMA3A | 10277194 | 23984752 | 7 | 83388387 | A | G | ATGGGTAGCATATANCATTGACTCAGGTC |
| 849 | SEMA3A | 10261415 | 24424210 | 7 | 83405532 | G | T | TGTGTTTTTGCCTCNAAGAGGGATTTGCC |
| 850 | SEMA3A | 2040874 | 23985068 | 7 | 83413735 | T | C | GAAATTCAAGATTCNTATATGTAAAGTCC |
| 851 | SEMA3A | 17301441 | 24424417 | 7 | 83423543 | C | T | CTCACAATGTCAAANGTCAAATTCAGAAC |
| 852 | SEMA3A | 464 | 24424435 | 7 | 83431111 | A | G | TTTAAATGGCAGTANAAGAAATACTGATT |
| 853 | SEMA3A | 17242340 | 24424496 | 7 | 83443943 | C | T | TAAAAATAACATGCNCTCATTAACTGAGA |
| 854 | SEMA3A | 17302032 | 24424584 | 7 | 83453363 | C | T | CTCCAGACAGTGTGNTTCTACTAGAAAGC |
| 855 | SEMA3A | 10256270 | 23985411 | 7 | 83466053 | A | G | TGGTGGATCTTTGANAACAAGTGACCATG |
| 856 | SEMA3D | 2040957 | 23365843 | 7 | 84271039 | C | T | TAATTATCATAAATNAAGAGTATTGTATC |
| 857 | SEMA3D | 10486845 | 24471674 | 7 | 84283783 | G | A | TTTACCTGTCTATTNCACAACTGTTATTG |
| 858 | SEMA3D | 7793649 | 23469525 | 7 | 84291120 | A | G | TCTGGTTGGCATATNCTCCTCCATTCCAT |
| 859 | SEMA3D | • | 46545325 | 7 | 84297153 | T | C | GTCTGCAAAGAAACNGTTAGGGAATTTTA |
| 860 | SEMA3D | 17159577 | 24023490 | 7 | 84310613 | G | A | ACATAAACATGTTGNGGATAAGCTCCACA |
| 861 | SEMA3D | 2286190 | 24472056 | 7 | 84334644 | A | T | TTTCTCCCTATGAANCTCTTTTCACCCAG |
| 862 | SEMA3D | 16887821 | 23470078 | 7 | 84340965 | G | C | CTT7CCATCCTCTANAGTATTTCCCTACA |
| 863 | SEMA3D | 2286192 | 23470180 | 7 | 84349360 | T | C | AGGGCAACATTTTANACACAATGGGGATA |
| 864 | SEMA3D | 12176601 | 24023758 | 7 | 84358394 | A | T | CTGGTTCTTCAATANTACAGTGATTTTAC |
| 865 | SEMA3D | • | 46544528 | 7 | 84368277 | T | C | ACAAGTGAGTGAGANACATTCAGACTTTT |
| 866 | SEMA3D | 17647248 | 24472505 | 7 | 84389473 | A | T | GTGATATTCACTGTNTTAAAGAAACCGGA |
| 867 | SEMA3D | • | 46545324 | 7 | 84396602 | T | C | CAAAAGCTGTCATTNTGAGACAAGTTCTC |
| 868 | EPHB4_ZAN | 314344 | 23451218 | 7 | 100039654 | G | C | ATCTAACCTGGTGGNCGTCCTACTGGGAC |
| 869 | MET | 40238 | 24454076 | 7 | 115908278 | A | C | GATTAATCATAGCCNGTCAATACGAAAGC |
| 870 | MET | 38842 | 24009444 | 7 | 115914356 | T | A | TTTTAAAATTCCAANGTAAAGGTCTCACA |
| 871 | MET | 39748 | 24009460 | 7 | 115921381 | C | G | ATCTATTTGCTCAGNATTCTCTATTCTTT |
| 872 | MET | 2237712 | 23445687 | 7 | 115932609 | A | G | TCTACATGCGCTCANGAAAACAATGCCTG |
| 873 | MET | 10243024 | 23445816 | 7 | 115940554 | G | A | TATATTTTTACTCCNAATACTGTTTCACA |
| 874 | MET | • | 46545044 | 7 | 115959364 | T | C | ATGTTTCAAAACTGNTTAGGACATCAAGA |
| 875 | MET | 38859 | 24009695 | 7 | 115973045 | C | T | GGGCTGTGCTGAGCNTCCTCCACCTTCAT |
| 876 | MET | 11770163 | 23446130 | 7 | 116011799 | G | C | TTGGAAGCATGTTGNCACCTACAGTAGTA |
| 877 | MET | 2073560 | 24009830 | 7 | 116017112 | 6 | A | CAGCCTGTTGAATTNGCAATGTCAATGTC |
| 878 | MET | 41741 | 23446303 | 7 | 116032462 | G | T | AGGAAGCCTAAGATNGATTTCACCTGGCT |
| 879 | MET | 41749 | 24009939 | 7 | 116040833 | C | A | ATTTAGAGGGAACGNAAATTAACCAAATC |
| 880 | EPHB6 | 10952539 | 23370649 | 7 | 142062195 | A | 6 | AACAGGTCATGATGNTTCAGAAAAGCAAA |
| 881 | EPHB6 | 7792701 | 23370680 | 7 | 142075187 | A | G | GAAGGATCCTAGTANCATTTCAGACATTT |
| 882 | ABCB8_ACCN | • | 46544761 | 7 | 150181468 | A | G | TTCAGGGGTGTGATNCAGTGCATTGATGG |
| | 3_CDK5 | | | | | | | |
| 883 | PPP3CC_SLC3 | 4872496 | 24223236 | 8 | 22345233 | G | A | GGACATCTGGCCAGNTGGAGAGATCCACC |
| | 9A14 | | | | | | | |
| 884 | PPP3CC | 4871998 | 24488206 | 8 | 22364095 | A | T | CGTGATCTGTGAACNATATCAAGGGTCAT |
| 885 | PPP3CC | • | 46547091 | 8 | 22392834 | C | T | ATATATTTTGTTTTNAAGAAAATTGAGAG |
| 886 | PPP3CC | 2469758 | 24488313 | 8 | 22418977 | C | T | AGATTGTGGTATATNCATAAAGCGAAATA |
| 887 | PPP3CC | 2461489 | 23492507 | 8 | 22426198 | G | A | TTAGGATGTAAGAGNGTCTTTTACTACAG |
| 888 | PPP3CC | • | 46547090 | 8 | 22435801 | G | A | AAACAGAATGATGANTACTAAGCTATTCA |
| 889 | PPP3CC | 2449340 | 24488612 | 8 | 22445845 | T | G | GAACAGTTTTTTTANAATTGGAATGTAGT |
| 890 | PPP3CC_SCA | 2249098 | 24488709 | 8 | 22456043 | T | C | TTATGCTCCTTTCTNTTCCAATCCCAGGA |
| | M-1 | | | | | | | |
| 891 | DPYSL2 | 17320156 | 24537503 | 8 | 26481651 | 6 | C | GAGACCAGTCGTTGNGTTGATGACACTGT |
| 892 | DPYSL2 | 7827953 | 23564653 | 8 | 26488872 | A | G | TGCGCTGAATGGTANATATGGTAAGAAAT |
| 893 | DPYSL2 | 327234 | 24537742 | 8 | 26500586 | T | C | GCTGGGATTTGCTTNACAGAGAAGGAGGC |
| 894 | DPYSL2 | 327232 | 23565023 | 8 | 26511632 | G | A | TTTTATATTGACCCNTGGCTACTGTGGTA |
| 895 | DPYSL2 | 17321828 | 24537981 | 8 | 26527954 | G | A | TAAGGGCACAGTTTNTATCTTCTACCTCT |
| 896 | DPYSL2 | 7004868 | 23565428 | 8 | 26546740 | C | G | GGATGGAGAGGGAGNCAGTGATGTTCTGA |
| 897 | DPYSL2 | 13277175 | 24538303 | 8 | 26553930 | G | T | GTGTCTGTGGCCTGNCTGAGCTCCGGAAG |
| 898 | DPYSL2 | 6557934 | 24078789 | 8 | 26566709 | T | C | CTGTATGACGGACCNGTGTGTGAAGTGTC |
| 899 | DPYSL2 | 7827731 | 23565895 | 8 | 26571810 | A | G | CAGCCTGCGGGTGCNTGGCAAACCAAGTG |
| 900 | DPYSL2 | 17055673 | 23565935 | 8 | 26580338 | C | T | TCCTCTCCCCTGTGNATCTTTTCTCAAAG |
| 901 | UNC5D | • | 46546978 | 8 | 35515328 | T | C | AAGAGAGTTTAGATNCCTAAAAGAATTGA |
| 902 | UNC5D | 7833633 | 23553518 | 8 | 35529967 | A | G | ATAAGCAAAAGGCTNCAAAACAATGGCAG |
| 903 | UNC5D | 2579895 | 23553623 | 8 | 35535572 | G | A | GAAGTATAGTTTTCNATGCTCTGATCTTG |
| 904 | UNC5D | 6468320 | 23553690 | 8 | 35543622 | C | G | TTGTTAAGAGGCACNTGAGCATAGTGCTG |
| 905 | UNC5D | 2053343 | 24529879 | 8 | 35551808 | A | G | GATGCAGCCTTCCTNTACATTTCCATTCT |
| 906 | UNC5D | 2589346 | 23553826 | 8 | 35559341 | A | T | TTTCACCGCCATTGNTCATTCACTTTTCC |
| 907 | UNC5D | • | 46546977 | 8 | 35570418 | G | A | CTGGCTGAATACAGNTTAATTGGTGTCAA |
| 908 | UNC5D | 2918959 | 23553994 | 8 | 35589303 | C | T | GGGAAGGTCAAGGGNTTTTGTCAATGCAG |
| 909 | UNC5D | 2978581 | 23554165 | 8 | 35603766 | T | C | ATCATCCAGGCATCNGCGTGCTCTTGCAA |
| 910 | UNC5D | 13256961 | 24530216 | 8 | 35613128 | G | A | GGCACAACCTCAGANTGATTATATTTTTG |
| 911 | UNC5D | • | 46546976 | 8 | 35627942 | A | G | CCTGATGGTTTTCANTGAGAATGAGATAC |
| 912 | UNC5D | 1436612 | 23554483 | 8 | 35648583 | C | G | GACAAGTTTAGTCANAGCTTTATTCCTAA |
| 913 | UNC5D | 10112630 | 24530487 | 8 | 35654668 | G | A | CCCAGAAAATACCANACTGATTTCCTCAT |
| 914 | UNC5D | 11985782 | 23554695 | 8 | 35668723 | U | A | ATGACTGTCAAGAANCAAGAACTTGTTGA |
| 915 | UNC5D | 16884240 | 24072125 | 8 | 35674897 | T | C | ATGAAGCTGAAAAANAATTTGGAAGTCAA |
| 916 | UNC5D | 16884271 | 24072232 | 8 | 35681523 | C | T | TCTCAAATGTGTACNGTACAGGATTTGCT |
| 917 | UNC5D | 12675805 | 23554928 | 8 | 35691847 | C | A | TATGGAAACAATCANATGATGTAUCAGTA |
| 918 | UNC5D | 16884299 | 23554997 | 8 | 35700158 | A | G | TAUAAAAAAATGAANCCGTCTCTTTTCAG |
| 919 | UNC5D | 10503983 | 23555141 | 8 | 35706965 | A | T | GCTTACAAATGTGANTCATAAATGACTTG |
| 920 | UNC5D | 16884351 | 24072446 | 8 | 35712468 | T | C | ACTAAAATTGGAAANGTTGTAAGTCATCT |
| 921 | UNC5D | 16884371 | 23555296 | 8 | 35719590 | C | T | TTCCTGAGTGATACNCTGGGTAAAGGTGA |
| 922 | UNC5D | 16884403 | 23555477 | 8 | 35742984 | C | T | AGCGTGAGGATTACNAGAGGCATATGTGG |
| 923 | UNC5D | 6468338 | 24072757 | 8 | 35752828 | G | T | TGCTGCTCTCAGTTNCAAAAGGGATCCAG |
| 924 | UNC5D | 6987079 | 23555650 | 8 | 35769426 | T | G | ATATTAGATAGAAGNCTTTGCTACATGTA |
| 925 | PTK2 | 6991311 | 24049292 | 8 | 141733067 | T | C | TTCAGTTGAGCTTCNTGACTCGTTTCTAG |
| 926 | PTK2 | • | 46546213 | 8 | 141758154 | C | T | ACAGACAATGTCTANTGGCTGTGCTGTAC |
| 927 | PTK2 | 7820179 | 24503556 | 8 | 141770658 | C | T | TTTCATCTATGTAGNAATATGTGATAGCA |
| 928 | PTK2 | 4961287 | 24503572 | 8 | 141775502 | G | A | TACACTTTAGTGGANGAGACAGGAAAACA |
| 929 | PTK2 | 4543590 | 24356271 | 8 | 141795016 | T | C | GTAGAGTGCAGCAANCTGCTTATAGAAAA |
| 930 | PTK2 | 4445273 | 24356296 | 8 | 141819855 | C | T | CCTAATGGAATGTANACTTATTAACCTTT |
| 931 | PTK2 | 6993266 | 24356318 | 8 | 141831841 | G | A | ACAAACATGTGTTANAGATACACCATGTG |
| 932 | PTK2 | 4554515 | 24356383 | 8 | 141847875 | T | G | AGCCTTAGAGTCTCNCTTAATCCTCTGAT |
| 933 | PTK2 | 7843014 | 24356399 | 8 | 141859663 | A | C | ATGTACATCACCTANGAAGTATTCTTGTT |
| 934 | PTK2 | 7837141 | 23319494 | 8 | 141867413 | G | A | CTCCAAGAAGTCACNATTAAGCTTATAAG |
| 935 | PTK2 | 4961294 | 24356454 | 8 | 141884416 | A | C | GTCATCTCCCTGGANCTCAGACTCATGCA |
| 936 | PTK2 | 7010459 | 24356496 | 8 | 141900423 | T | C | ACCAGAGTGAGAGGNTGAGGTAAGGCAGA |
| 937 | PTK2 | 4961234 | 23932330 | 8 | 141910063 | T | C | TTTCTTTTTTTACCNTTATTGTTGAAAAC |
| 938 | PTK2 | • | 46546226 | 8 | 141926504 | A | C | CAGTGAAAAGGCAANGGATTTACGTACTT |
| 939 | PTK2 | 7008498 | 24356661 | 8 | 141946210 | A | G | ATGATGATATCTGANCAAAGATCTAAATA |
| 940 | PTK2 | 7843337 | 24503712 | 8 | 142031824 | T | C | AGAACAAAGAAAGANGTGAACTGACACTC |
| 941 | PTK2 | 1031262 | 24503837 | 8 | 142055087 | T | G | CACATCATGCTGCANGCCAGGGAACTATA |
| 942 | SEMA4D | 7465881 | 24558872 | 9 | 89233303 | A | G | CACCTGGCGGCATCNAGGGAGCAAGGACG |
| 943 | SEMA4D | 6559349 | 23592645 | 9 | 89236779 | G | T | CCCCACAAACAGGGNCTAACACAAGCTCG |
| 944 | SEMA4D | 4074459 | 24455444 | 9 | 89264492 | G | T | GTGGTTTAAGACACNTATCATATTCACGG |
| 945 | SEMA4D | 4078338 | 24010605 | 9 | 89276987 | T | C | CTCTGAGTGCCCTGNCTCTATCTCTGGTC |
| 946 | SEMA4D | 9330435 | 23447477 | 9 | 89291592 | G | A | GTCCTTCCAATCTCNATGGAGGCACCCAA |
| 947 | SEMA4D | 9969727 | 24010676 | 9 | 89304388 | T | C | CAACTGCAACAATGNCAAGTGCTCCTAGA |
| 948 | SEMA4D | 4405007 | 24455704 | 9 | 89330358 | C | T | TCAGTATAGAGCATNGAATCACAACTCCT |
| 949 | GRIN3A_PPP3 | 3739723 | 24052211 | 9 | 101433905 | T | A | TCCTCCATGTAAAANTCCTCCACAAAAAG |
| | R2 | | | | | | | |
| 950 | RGS3 | 16933949 | 23518179 | 9 | 113304006 | G | A | TCCAGCCAGGAAGANGATCACGCATGCCA |
| 951 | RGS3 | • | 46547548 | 9 | 113321261 | T | C | TGTCACCTTTCCTCNAGTTTTTTCCACCA |
| 952 | RGS3 | • | 46547547 | 9 | 113343568 | T | C | GTCTTCTTTGACCTNTCCTGCTCCCTGCA |
| 953 | RGS3 | 7026656 | 23518472 | 9 | 113359657 | C | T | CTTTCAAAAGCTGTNGAATCCTTCTTTGA |
| 954 | RGS3 | 16905607 | 23518477 | 9 | 113362505 | T | C | GTCAGCGACACGTTNTGTGGGACCCTGAG |
| 955 | RGS3 | • | 46547546 | 9 | 113399214 | A | G | CAATCATTTCATCANTAGTCCCTGGGCCT |
| 956 | RGS3 | 4979254 | 23518785 | 9 | 113411796 | T | A | GAGGTGTAGACATGNTTCCCGCCTATCCA |
| 957 | RGS3 | 2812398 | 24506291 | 9 | 113447379 | T | C | CTTATTCTCTCTTANGCTTCTGTAGTCAA |
| 958 | ABL1 | • | 46547438 | 9 | 130620282 | C | T | AAAAATCAGTTGAGNCCACCTATCCTATA |
| 959 | ABL1 | 4740359 | 23586699 | 9 | 130624773 | G | A | GGAAGCCAAGAATANCGTTGGTGTTGTTG |
| 960 | ABL1 | 2791725 | 24554227 | 9 | 130637510 | A | G | GGAAATGACTAACANCTGCTTAAGTCCTT |
| 961 | ABL1 | 2791731 | 24554327 | 9 | 130647425 | T | C | TAACCCATCTTAGCNAGACTCCAGGTTTC |
| 962 | ABL1 | 2855169 | 24554449 | 9 | 130660455 | T | C | CACTCATCACTATCNGCATGTTCGTGGTC |
| 963 | ABL1 | 11244131 | 24092330 | 9 | 130665206 | C | G | TAAACAGTAGCTCCNTCCTTAACATCAGT |
| 964 | ABL1 | 2791745 | 23586980 | 9 | 130673294 | G | A | CAGCAACTCAGAGCNTATGGAAGACTGTC |
| 965 | ABL1 | 2855172 | 24554570 | 9 | 130681202 | C | A | GTTAATACTCAATCNTCTGTGAAATTCAA |
| 966 | ABL1 | 9695221 | 23587073 | 9 | 130689849 | A | G | TCCGTGTTGGGATGNGTGGAAGGTTGCAT |
| 967 | ABL1 | 10901285 | 24554670 | 9 | 130700428 | G | A | TTATGAATTACGTTNTGTCCAGTTATGAT |
| 968 | ABL1 | 11244146 | 23587166 | 9 | 130704697 | G | A | AATAGTAACTCCTCNGAGACTTCCAGGGA |
| 969 | ABL1 | 11244149 | 23587176 | 9 | 130713731 | C | G | GTCAAAACTGAACTNTTAAAATTCTTACA |
| 970 | ABL1 | 17147263 | 23587190 | 9 | 130725679 | T | C | GCATTTCTAATCCTNTGTCTCATCATTTA |
| 971 | ABL1 | 6597642 | 23587208 | 9 | 130755321 | G | A | GACAGTGGGAATGCNTAGTGAATGGCTCT |
| 972 | ABL1 | 1800609 | 23587249 | 9 | 130760738 | T | C | GCATAAAGAGATCTNGCCCGATGTCAACC |
| 973 | ABL1 | 17147276 | 24092583 | 9 | 130768163 | C | T | TCATTCTCATAGTCNGAGTGTGTTTCCAC |
| 974 | ABL1 | 12005009 | 23587437 | 9 | 130785308 | T | C | GGCTGTTTCTCCGGNATCCACGTGCCTTT |
| 975 | ABL1_P518 | 2855197 | 23587499 | 9 | 130793960 | A | G | TTCCAGGTCATTTCNAGTCCTGCCTTCGA |
| 976 | ABL1_FIBCD1_ | 7853654 | 24092734 | 9 | 130799824 | G | A | AAGAAAACCTTTTTNACAATGACCGCGGG |
| | P518 | | | | | | | |
| 977 | ITGB1 | 2000217 | 24147441 | 10 | 33222000 | A | G | CCAGAGGTTTGITCNTAAGTCAATAGGTG |
| 978 | ITGB1 | 2488315 | 24147448 | 10 | 33229095 | G | A | ACCTCTCTCCCAGCNTAGCAGGATAAGTA |
| 979 | ITGB1 | 2230396 | 23678328 | 10 | 33249272 | G | T | TAACTTATTGTTACNCCTTCTGACAATTT |
| 980 | ITGB1 | 16933790 | 24147450 | 10 | 33251010 | T | C | CTTTGTTTAGGCCANGAATCTTGCTTTTG |
| 981 | ITGB1 | • | 46548998 | 10 | 33269789 | A | G | CTTTAACACACTCTNCTCCCTTGAACTCT |
| 982 | ITGB1 | 1187071 | 24147454 | 10 | 33281583 | C | T | GTGACTTCCATACTNCAAATGGTACATAC |
| 983 | NRPL | 6481838 | 24550790 | 10 | 33500750 | C | T | GTGTCATCCTGGTANAGAGTAATTCCAGA |
| 984 | NRP1 | 10080 | 23582752 | 10 | 33507114 | G | A | GAATGGACTGTCTCNTCTATGATGCAAAT |
| 985 | NRP1 | • | 46548997 | 10 | 33514250 | A | G | TCCACTGTTCATCANAATGTTTCGGTATG |
| 986 | NRP1 | 10827212 | 24089769 | 10 | 33536295 | A | G | ATCCGAGGAGAGTCNTGCAAACAACCCAA |
| 987 | NRP1 | 2070303 | 24089802 | 10 | 33542246 | C | T | AAGAAGCCCTTCCTNGGGAGAATGCCAGA |
| 988 | NRP1 | 2254826 | 24551199 | 10 | 33544471 | G | T | GTCCAACACTTCAANTATTTTCCTTTAGA |
| 989 | NRP1 | 11009311 | 24089996 | 10 | 33560360 | C | A | CCCATGCAAAGTACNTGTGAATATTTGTA |
| 990 | NRP1 | 1888690 | 24090040 | 10 | 33575802 | G | C | TAACCTGGTCCTATNTATGCTCTGGATCA |
| 991 | NRP1 | 10763919 | 24090120 | 10 | 33581712 | T | A | ATCTAGAGATGGATNCAGCAATCCCAAGG |
| 992 | NRP1 | 10490938 | 24551677 | 10 | 33588382 | C | I | GAAGTACTCTTATCNGTCTCGTGTGTGTG |
| 993 | NRP1 | 1331312 | 24624588 | 10 | 33595519 | G | C | AGAAGCACTTTCTCNCAAAAGGACAATGG |
| 994 | NRP1 | 10827227 | 24147500 | 10 | 33601794 | C | T | CAAATTCTAGCTCANGTTGCCTTCCAAGC |
| 995 | NRP1 | 10827229 | 24624594 | 10 | 33607195 | C | A | TTCGQCAATCTTATNACCCAAACTCAGAT |
| 996 | NRP1 | 1331325 | 23678393 | 10 | 33620175 | T | C | GCAGGCTGATTTGANGGTTTTTGTCAGCG |
| 997 | NRP1 | 2776930 | 23678395 | 10 | 33627887 | T | C | CTACTTCTACCTACNATGCATTGAGCACT |
| 998 | NRP1 | 11597915 | 24624602 | 10 | 33633828 | A | G | TATGTCTTGAGTCTNAGCAATGACATTCA |
| 999 | NRP1 | 2776934 | 24147505 | 10 | 33639848 | C | G | CTGTTCAATTTGTGNCACTTGAAGAGTCA |
| 1000 | NRP1 | 11009338 | 24624606 | 10 | 33646312 | A | T | CTGTCATAACCAGANATGCAGGAAGGAGA |
| 1001 | NRP1 | 10827235 | 24147512 | 10 | 33652884 | C | G | TAAGTGCCTGCTCCNTAAAGTGAAATCTC |
| 1002 | NRP1 | 2026319 | 24147515 | 10 | 33661508 | C | G | TTCTGAGAAAGTGANAGTGTAATAAGATA |
| 1003 | CXCL12 | 266102 | 23579580 | 10 | 44176648 | C | T | TCCCTCCATGGCTGNGGCTCAGACACACA |
| 1004 | CXCL12 | 266096 | 23579675 | 10 | 44182463 | T | A | TTCTTTTTAGTTTCNTGGGCCAAGGGGTG |
| 1005 | CXCL12 | 1029153 | 24548898 | 10 | 44187152 | A | G | CAATTCAAGATTACNTCACTATAATTTAC |
| 1006 | UNC5B | 10762430 | 23606210 | 10 | 72644934 | G | T | TTTGGGTTTTTCCANTGGATCCTTCACAA |
| 1007 | UNC5B | 10999736 | 24569099 | 10 | 72659151 | C | G | CGGAAGGTCCTGCTNCAAGCCTGAGAATT |
| 1008 | UNC5B | 7076430 | 24104024 | 10 | 72675756 | G | A | CTCTGTATCTGAGCNAGGGTGTGCGGAAG |
| 1009 | UNC5B | 10740372 | 24104157 | 10 | 72701391 | C | T | AGCTCTGTAAGCAGNGTGGCCCCCATAGC |
| 1010 | UNC5B | 16928594 | 24104193 | 10 | 72706905 | T | C | CTGCATTAGAGTTANAAAACAAAGTCATA |
| 1011 | UNC5B | 10823719 | 23606624 | 10 | 72719470 | A | G | CCCAGTGTCAGTGANGGCATTAGGAGGCA |
| 1012 | UNC5B | 10823720 | 24104341 | 10 | 72727761 | G | C | TTCAAGATCCCACTNTCCATCCGCCAGAA |
| 1013 | UNC5B | 17628806 | 24569522 | 10 | 72735101 | A | G | TTCCCTTGCTTGCTNTTACTAAGACGGTC |
| 1014 | SLC29A3_UN | 7079524 | 23606893 | 10 | 72739498 | A | G | ACACCACTGGAGATNGTCAGGACAGGGAC |
| | C5B | | | | | | | |
| 1015 | PPP3CB_ZMY | 2297452 | 23630748 | 10 | 74856618 | G | C | AGGGAGAGGAATGANAGCTGATGCACCAA |
| | ND17 | | | | | | | |
| 1016 | PPP3CB_ZMY | 12644 | 24118870 | 10 | 74867057 | C | T | ACCCTGGTGGCTCTNTAAGCAACTTGAGT |
| | NDI7 | | | | | | | |
| 1017 | PPP3CB | 12571454 | 24118909 | 10 | 74878961 | T | C | AATGTAGGTAACTTNATAAACTGCCTCAG |
| 1018 | PPP3CB | 11000666 | 24118943 | 10 | 74903000 | G | T | TGTATTTTTCTCCTNTTGCTGCAAGTCTC |
| 1019 | PPP3CB_USP5 | 3793663 | 24118964 | 10 | 74930189 | T | C | GTCATTTTCGTAAGNAGACAGACTCTAGG |
| | 4 | | | | | | | |
| 1020 | C10orf12_SLIT | 10882861 | 24088924 | 10 | 98741433 | C | T | TTCTCTTAAAAATANCAGCATGTCTGTGG |
| | 1 | | | | | | | |
| 1021 | SLIT1 | 4917753 | 24089004 | 10 | 98756580 | C | A | ATGGATTCTTAAAGNGACAGAAGATGCTC |
| 1022 | SLIT1 | 7896883 | 24089077 | 10 | 98777344 | T | C | CTTTCTACAAGTACNGCTAACCTGATCTT |
| 1023 | SLIT1 | 7924206 | 23581980 | 10 | 98810099 | A | G | TGCGCTCCATTATCNCAGGCATTTGTAGC |
| 1024 | SLIT1 | 12268671 | 24550432 | 10 | 98830669 | T | C | TATGGCCTGGTACCNGATCTCAGGAGAAG |
| 1025 | SLIT1 | 2817686 | 23582079 | 10 | 98837657 | C | G | CTACCCAAAGTTTGNTAAGAAAATGTGCT |
| 1026 | SLIT1 | 17112401 | 23582140 | 10 | 98845455 | G | A | CCCAGCTCTATGACNGACATCATGTGGAT |
| 1027 | SLIT1 | 2817692 | 23582188 | 10 | 98855276 | A | G | TGTAATGAGAGCAGNGAACATAAATCACC |
| 1028 | SLIT1 | 2817701 | 24550638 | 10 | 98864103 | G | A | CTGAAGTCTTCCTANTAAGATCAAAAGTA |
| 1029 | SLITI | 2817703 | 24550648 | 10 | 98868116 | C | T | CCTCCACTTCTTGGNGCAGCAGGACTTGG |
| 1030 | SLITI | 2484879 | 24550696 | 10 | 98878639 | G | C | GCCCCATTCCTTCCNTGTTCCTTAAATAT |
| 1031 | SLIT1 | 2817711 | 24089427 | 10 | 98885824 | G | C | TGGTGATAAATAAANAATGGCAACACACT |
| 1032 | SLIT1 | 17112452 | 23582494 | 10 | 98893384 | T | C | TAGAAAAGCCACTANTGATGAGAGGTAGG |
| 1033 | SLIT1 | 2784920 | 23582570 | 10 | 98903618 | G | T | TATTCAGAAATAACNAGAAATGGCCCTCA |
| 1034 | SLIT1 | 17502307 | 24550831 | 10 | 98910830 | G | A | CGATCAACTGATGCNCCACTGGTCTCCAA |
| 1035 | SLIT1 | 7073919 | 24550843 | 10 | 98916951 | A | G | CTCTGCTTAATGTANAGGTCATTTTTCAA |
| 1036 | SLIT1 | 2817666 | 23582713 | 10 | 98923843 | G | A | GCTTCAGGCCTCCANTCAAACGCCCTCTT |
| 1037 | C10orf6_SEM | • | 46548573 | 10 | 102712906 | G | C | TCATCTTTCCCCCCNACAACTGGAGACAC |
| | A4G | | | | | | | |
| 1038 | C10orf6_MRP | 4919510 | 24037660 | 10 | 102724768 | C | G | GTGTTGGGACAGCTNCGTTTAAAAAGGCA |
| | L43_SEMA4G | | | | | | | |
| 1039 | MRPL43_PEO | • | 46548572 | 10 | 102736221 | C | T | TTAAAAAACAAGGGNCCTGTAAAACCCTT |
| | 1_SEMA4G | | | | | | | |
| 1040 | LZTS2_MRPL | • | 46548570 | 10 | 102740773 | C | A | TGAGTGGCCTTTAGNGGAGCTCAAGCTTT |
| | 43_PEO1_SEM | | | | | | | |
| | A4G | | | | | | | |
| 1041 | ABLIM1 | 17406965 | 23640225 | 10 | 116173637 | G | A | ATCTGGGCTAGTTTNCCCCTCCTTAGGCA |
| 1042 | ABLIM1 | 12218918 | 24124965 | 10 | 116180806 | T | C | TTTTTAAAATGAGANAGACCTTCAAAGGG |
| 1043 | ABLIM1 | 4751908 | 24595682 | 10 | 116188360 | T | C | GGGTGCATTTGTGCNGGGGTTAACAATCT |
| 1044 | ABLIM1 | • | 46548493 | 10 | 116193964 | A | G | TGCAGTTTCTTTGTNGCAGATCCCACAAG |
| 1045 | ABLIM1 | 7080135 | 24595772 | 10 | 116204106 | A | C | CTCAGATGAAGGTGNCACCAATTCTTCAA |
| 1046 | ABLIM1 | 11196754 | 24595847 | 10 | 116222151 | C | G | GTCCAGGAAACTAGNAGCAGAATGGCTGT |
| 1047 | ABLIM1 | 2145896 | 24595889 | 10 | 116232258 | C | A | TACTTAAAAGATAANATATCTGTGACTTG |
| 1048 | ABLIM1 | 17717312 | 24595949 | 10 | 116247895 | G | A | TTTACCATCCATAGNGCCTCAAATATGTC |
| 1049 | ABLIM1 | 12765660 | 23640814 | 10 | 116258629 | C | A | AATTGAAAGTTCTANAAACAGATGAAGAT |
| 1050 | ABLIM1 | 2475242 | 24125352 | 10 | 116275793 | A | T | TTCTTTTACTGCATNTCAAAGGTGGAAAG |
| 1051 | ABLIM1 | 11196784 | 24125406 | 10 | 116295130 | A | G | TCTCAGAGCTAACTNGCAUAAAGCCTGGC |
| 1052 | ABLIM1 | 2483556 | 24125450 | 10 | 116311230 | G | A | AAAACATAGGGTGGNAAATACACTAAAAA |
| 1053 | ABLIM1 | 2483567 | 24596340 | 10 | 116322760 | A | G | ATTATTTTAGACCCNAGTCCCACACGAAT |
| 1054 | ABLIM1 | 2483580 | 24125577 | 10 | 116337328 | A | G | GCCATCTTTTCAAGNACAATAGGAGCATC |
| 1055 | ABLIM1 | 2253338 | 24596450 | 10 | 116347520 | C | T | CTGCCTTTGGGTAGNGGTACTATGTAAGA |
| 1056 | ABLIM1 | 2483589 | 23641358 | 10 | 116355306 | C | T | CCCAGCACTTTCCANTGTACTGACTAGTC |
| 1057 | ABLIM1 | 2497668 | 24125697 | 10 | 116363265 | C | T | GGCACACCATTGTGNCAAAGGTTATACTG |
| 1058 | ABLIM1 | 11196811 | 24125729 | 10 | 116374052 | C | A | TCCTCTTTCTTGACNCCTAGGGCTGTGAA |
| 1059 | ABLIM1 | • | 46548303 | 10 | 116387416 | C | A | ATTTAAAGTTCCCTNTAAACAAATCACTA |
| 1060 | ABLIM1 | • | 46548479 | 10 | 116393696 | T | C | ATTTCTACAGCATGNTTCTTTCGGACAGA |
| 1061 | ABLIM1 | 17092220 | 24125860 | 10 | 116402184 | C | T | AAGAGGTTCTCTCANTTACGTGATACCCT |
| 1062 | ABLIM1 | 11196850 | 24596747 | 10 | 116415129 | A | G | TCTGGGCCTTGAAGNCAAACTAAGGGAAT |
| 1063 | DKFZp761L15 | • | 46550486 | 11 | 532844 | T | C | ATTACACCTGTACANGACTCTAGCCATG |
| | 18_HRAS | | | | | | | |
| 1064 | RRAS2_SPON1 | 4756787 | 23593529 | 11 | 14248953 | C | A | GAGGAAGTCAAAAGNGCCACCTGTGAAAA |
| 1065 | RRAS2 | 1431374 | 24096649 | 11 | 14260551 | T | C | GGTGGCATAGCTGANTGATGTATAAAATG |
| 1066 | RRAS2 | • | 46550381 | 11 | 14266858 | G | A | AGTTCAAAAACACANGTAGCAGCAGCAGT |
| 1067 | RRAS2 | • | 46550380 | 11 | 14274839 | C | A | TTCATTAGTAGGAANCTCAAACTTACTCT |
| 1068 | RRAS2 | 11023187 | 24559791 | 11 | 14302135 | T | G | GCAAACAACACTTTNTACAGTGAAACTCA |
| 1069 | RRAS2 | 2970332 | 23593842 | 11 | 14317011 | G | A | CATGATGGGGAAACNGTTTTTCACTAACC |
| 1070 | RRAS2 | 2915400 | 23593858 | 11 | 14321527 | T | C | TTGAATTACTAATANGCAACCTCACCATT |
| 1071 | NGL-1 | 10501220 | 24546853 | 11 | 40104412 | A | G | AGTCAGGACGTCTCNCCTAATGATATTAT |
| 1072 | NGL-1 | 11035708 | 23577166 | 11 | 40110341 | G | A | TATCAATTTCTTACNTGTTTAGCAAGTAC |
| 1073 | NGL-1 | 12273845 | 23577228 | 11 | 40116743 | G | A | GAACTGCCAGCTCCNGATTCATTTTCCTT |
| 1074 | NGL-1 | 17385222 | 24547038 | 11 | 40123689 | T | C | GGGACCTTATCTATNGATTACCTATACCA |
| 1075 | NGL-1 | 4429048 | 24547125 | 11 | 40149102 | G | A | TAATATGTAGCGAGNAAGTAGATCAAGGC |
| 1076 | NGL-1 | 6485187 | 24086441 | 11 | 40162079 | A | C | CTGAAAAAGACAATNATATAAATCATTAA |
| 1077 | NGL-1 | 1993167 | 24086536 | 11 | 40186158 | C | T | ACTTGCTTATGATANTACATCAGTCCATG |
| 1078 | NGL-1 | 10501222 | 23577835 | 11 | 40198300 | C | A | CTATGAATGGTAAANCTTTTGAGACTATG |
| 1079 | NGL-1 | 10837367 | 23577967 | 11 | 40204827 | C | T | CTGGTCCTAATAAGNGCTATGATCCTGAG |
| 1080 | NGL-1 | 6485190 | 23578042 | 11 | 40211317 | A | T | TGCCCACTCATTTTNTAGATTTCCACTTA |
| 1081 | NGL-1 | 1377107 | 23578285 | 11 | 40218796 | G | A | CTATGCTTATAAGTNATGGAGTTTTCAAC |
| 1082 | NGL-1 | 1020579 | 24087109 | 11 | 40237012 | G | A | ACACATCACCATAANGAATGACAGAATTA |
| 1083 | NGL-1 | 16934661 | 24087147 | 11 | 40243736 | A | C | GAATATTTCAGCAANACTTAACTCCTAAC |
| 1084 | NGL-1 | • | 46550172 | 11 | 40252910 | G | C | AACTTACATAGAGTNCTACAACCAGAGCA |
| 1085 | NGL-1 | 10501227 | 24548156 | 11 | 40262733 | A | G | AAGACTAGTTAGAGNCAATCTAGGAGAGA |
| 1086 | NGL-1 | • | 46550171 | 11 | 40268425 | G | A | ATAAGCAAACGAATNCAAAAAAAGTGCAG |
| 1087 | NGL-1 | 11035770 | 24087331 | 11 | 40275193 | T | C | GACTATAGCTGGTGNAATCATTAGCAAAC |
| 1088 | NGL-1 | 16934694 | 24087355 | 11 | 40281076 | G | A | GCTGGAACTTAAGGNAAATGTGTGGCAGG |
| 1089 | RHOD_SYT12 | 17148051 | 23598960 | 11 | 66579215 | A | G | TTCAGTGAAACTGTNTAGCATGACAAACA |
| 1090 | RHOD | • | 46549993 | 11 | 66590828 | C | T | CTGTTCTACCCTGANGCCAGCGTCCTGCT |
| 1091 | PAK1 | • | 46549428 | 11 | 76729331 | G | C | CCGTGAGGTAAGGCNAAGGGCCAAGCAGT |
| 1092 | PAK1 | 2602475 | 24129780 | 11 | 76751007 | G | A | TTCTTCTAGTGACANATCTCAAACGTCCT |
| 1093 | PAK1 | 2844336 | 24129792 | 11 | 76759373 | T | C | CAACAGTTGATTTANGTCATGCTTTTGTT |
| 1094 | PAK1 | 562939 | 24129805 | 11 | 76767859 | T | C | CTCTCATCCCAGACNAATAGGCATGTGAG |
| 1095 | PAK1 | 503406 | 23648055 | 11 | 76785784 | G | A | CCAATGCCTCAGACNTGCTGCAATATAAA |
| 1096 | DKFZp434E1 | 1476925 | 24129827 | 11 | 76793285 | T | C | TTTCAATGAGAAAANAGAAATCTTGGCAG |
| | 19_PAK1 | | | | | | | |
| 1097 | DKFZp434E11 | 535008 | 24601873 | 11 | 76801862 | G | A | AAGTTTTAGCAGTGNGAAGGCTTTGAAAG |
| | 19_PAK1 | | | | | | | |
| 1098 | DKFZp434E11 | • | 46549922 | 11 | 76819366 | T | C | CCAGTTGTGATTGCNGACTCTTCCCCCTT |
| | 19_PAK1 | | | | | | | |
| 1099 | DKYZp434E11 | 17135665 | 24129857 | 11 | 76825556 | A | C | ACTGCCTGCTTCTTNGTATAGCTGGAAGC |
| | 19_PAK1 | | | | | | | |
| 1100 | DKFZp434E11 | 7112264 | 24129866 | 11 | 76839389 | T | G | AGAGGACTTAGACANGGTTCCAGTTTTTG |
| | 19_PAK1 | | | | | | | |
| 1101 | DKFZp434E11 | 17135689 | 24129892 | 11 | 76853900 | A | T | ACCTACAATTTTTTNAAAAGTGCCACTGG |
| | 19_PAK1 | | | | | | | |
| 1102 | ARGGEF12 | • | 46549566 | 11 | 119773232 | A | G | CATAAAGATTTGACNTGAGTTCATGGAAT |
| 1103 | ARHGEF12 | • | 46549565 | 11 | 119778807 | G | A | TTTTAAGTCCAAAANAAATTCTGTTTAAC |
| 1104 | ARHGEF12 | • | 46549564 | 11 | 119784342 | G | C | TTTTAAAACATCTANGCTTTTTCCTTTTG |
| 1105 | ARHGEF12 | • | 46549563 | 11 | 119804917 | T | C | TTATCCTAGGATTANAGGAAGACTAATTT |
| 1106 | ARHGEF12 | 10502236 | 23681484 | 11 | 119812952 | A | G | ATGCAGTATATCTANTTATTTGGTAGAAA |
| 1107 | ARHGEF12 | 11217872 | 24627013 | 11 | 119826411 | G | C | GTGCCTCTTGTTGANAAACATTGAGAAGG |
| 1108 | ARHGEF12 | 11600200 | 23681494 | 11 | 119839890 | T | G | ACTAGTAAATCACTNTTTAGGTGTTTAAC |
| 1109 | ARHGEF12 | 11217878 | 23681497 | 11 | 119845593 | G | A | GCATAGGGTACAGANTAGTTGAGAAAATG |
| 1110 | ARHGEF12 | 2276035 | 23681503 | 11 | 119851570 | G | A | AAATTTACTGCTTANAAAGAGACCTAAGA |
| 1111 | ARHGEF12 | • | 46549562 | 11 | 119865481 | G | A | AGCTCAGAAGGCTCNCTTACTTTGGGCAT |
| 1112 | ROBO3 | • | 46549505 | 11 | 124230670 | G | C | TCAGTGGTGAAGACNTGCTTCCTGGTCCA |
| 1113 | ROBO3 | 3923890 | 24124283 | 11 | 124245620 | A | T | CCTGCCTGTCTTTANGTACCTTGACCCTG |
| 1114 | ROBO3_ROB | 12823 | 24363370 | 11 | 124259821 | T | A | GGTCAGCTTTGCTCNAATTTTGTTTTCAT |
| | O4 | | | | | | | |
| 1115 | KRAS2_LOC1 | 4246229 | 24659660 | 12 | 25258936 | G | A | GTTTCACAATGAATNATGGCAGCAACTGG |
| | 44363 | | | | | | | |
| 1116 | KRAS2 | 11047902 | 24436544 | 12 | 25271060 | C | T | TATACACACGATTGNTTTTAAGACTGTTA |
| 1117 | KRAS2 | 10505980 | 24436767 | 12 | 25283710 | C | T | TGTTACTTAGATAANACTGCAAGATCCAT |
| 1118 | KRAS2 | 10842518 | 24436886 | 12 | 25291473 | G | T | ACAGCATCATAATTNACTGAAGACCCAAA |
| 1119 | KRAS2 | 12320328 | 23423267 | 12 | 25299731 | A | G | TCCATATAGGCTACNTGCATACTGGGTAT |
| 1120 | DDX23_RND1 | 2446977 | 23396212 | 12 | 47532293 | A | G | CAGGCCGACACTGANGCCTGGAAACTACC |
| 1121 | RND1 | 4760597 | 24416092 | 12 | 47543869 | T | A | TTACGGTCCCTTTTNAAACTCAGCCTGAA |
| 1122 | SRGAP1 | 17099873 | 23960757 | 12 | 62526269 | A | G | CTTGTTTCATTGCTNTGGAAGTTGTGTTA |
| 1123 | SRGAP1 | • | 46551254 | 12 | 62547516 | A | G | GTTTCAGTGATTGANCTTCATTTAAAGAG |
| 1124 | SRGAP1 | 17099889 | 23367243 | 12 | 62568240 | T | C | CCAGTTACCAACTGNGTTCTCTTCTCCAA |
| 1125 | SRGAP1 | 3851606 | 23367318 | 12 | 62577391 | C | A | CAGCCAAATTTAGGNTTGATAATATTATG |
| 1126 | SRGAP1 | 4763132 | 23367362 | 12 | 62600425 | T | C | TCCTGAGATGTCCANGGAGTAGCAAGAGA |
| 1127 | SRGAP1 | 10784373 | 23960985 | 12 | 62610517 | G | A | ATGATAAAACATAGNAACTTTTAAAAGAT |
| 1128 | SRGAP1 | 7973936 | 23961021 | 12 | 62619912 | G | A | GAACCTAAAGATCTNTAAAGTCTCTGCAA |
| 1129 | SRGAP1 | 11175213 | 24393742 | 12 | 62626201 | T | C | TGAAATGGAGGTCTNTGTAATGAACAAGA |
| 1130 | SRGAP1 | 17099954 | 23367548 | 12 | 62633296 | A | G | GGGAGACAGAAGGTNAACCATATGTGGAG |
| 1131 | SRGAP1 | 4604983 | 23961084 | 12 | 62642438 | G | C | CCCCTTGACCAATGNTTTGGTGATTGGGA |
| 1132 | SRGAP1 | • | 46550560 | 12 | 62672284 | G | A | TATTTTGTAAGTCANAGTCAAGGGCTTGA |
| 1133 | SRGAP1 | 7957913 | 23961178 | 12 | 62680858 | G | C | TCCTATGGATGCGGNATTCTCTATTCCTG |
| 1134 | SRGAP1 | 17100009 | 23367914 | 12 | 62704085 | T | G | AAAACTCTCACACTNCATTCTATGATGGA |
| 1135 | SRGAP1 | 7295635 | 24394100 | 12 | 62711690 | A | G | AAATGTCATCCTACNCTATTTCCTCTGAG |
| 1136 | SRGAP1 | 7963940 | 23368092 | 12 | 62717554 | A | T | CTGCAACACTGTGGNTTCACCAACACCTA |
| 1137 | SRGAP1 | 990309 | 23368160 | 12 | 62734563 | G | A | AAGATTTGTCCATGNAAACCTGTCCCATT |
| 1138 | SRGAP1 | 1827073 | 23961407 | 12 | 62744468 | A | C | TATCAAAACCTAAGNTAAGTGACTTCTTG |
| 1139 | SRGAP1 | 1840664 | 23368244 | 12 | 62750105 | T | C | ATTTCAGCCACCGGNCCAGCATTGCTCTT |
| 1140 | SRGAP1 | 789709 | 24394326 | 12 | 62757574 | G | C | ATAAATCAGGAGGANTAAATGAAGTTAAG |
| 1141 | SRGAP1 | 1874314 | 23961521 | 12 | 62770981 | C | T | CCGGTTGTATCTTANTGTGTGACTTGTAA |
| 1142 | SRGAP1 | 11831686 | 23368511 | 12 | 62794201 | A | G | TCTGTAGTATATTCNCCAACTTTACACAT |
| 1143 | SRGAP1 | 789722 | 24394550 | 12 | 62806073 | T | C | TATGTTGGGCGGTCNGCCAGAGAACTATC |
| 1144 | SRGAP1 | 10748000 | 23368666 | 12 | 62826499 | T | C | GTGTCCTTAACATTNAGGAATGACATAGC |
| 1145 | SRGAP1 | 10748001 | 23961667 | 12 | 62832677 | A | G | CTACCTTTCTTTGANAATGTGATGCTGTG |
| 1146 | PLXNC1 | 17298648 | 24423613 | 12 | 93052018 | C | T | TCTATTGTGCTGTANGCCAGAACCAATTC |
| 1147 | PLXNC1 | 2365736 | 23984538 | 12 | 93066386 | C | T | CTCAGATTTTTAATNGAGTAATGGTAGAT |
| 1148 | PLXNC1 | 4761590 | 14902356 | 12 | 93076279 | C | T | AGCACTATAGGATTNGAGTCTCCCGATGG |
| 1149 | PLXNC1 | 3847812 | 24423763 | 12 | 93083855 | A | G | TGATCACGAACGGGNTTTGTTTGTTGCAC |
| 1150 | PLXNC1 | 17022174 | 23406188 | 12 | 93091688 | T | C | TGGThTTAGTGCCANGATGGAATCAGGGC |
| 1151 | PLXNC1 | 7137116 | 23406248 | 12 | 93097589 | T | G | CTGGTCAACCTCATNGCCCAAATCCCTTT |
| 1152 | PLXNC1 | 11107440 | 23406370 | 12 | 93103951 | G | A | TATGATTAAATACCNAAATATGTACTTCT |
| 1153 | PLXNC1 | 11107453 | 24424049 | 12 | 93111089 | C | T | TTTGAACAACTTAGNTTAAGCCGTTCTTT |
| 1154 | PLXNC1 | 11107458 | 23984870 | 12 | 93118453 | C | T | GTATTTGAGATCACNTTGTAAAGCTAGTT |
| 1155 | PLXNC1 | 10745681 | 23984928 | 12 | 93127171 | G | T | CCTATTAGATGCAANTATACCGACCTCCA |
| 1156 | PLXNC1 | 2361355 | 23984971 | 12 | 93132497 | A | G | GTGATGACATTAACNATGAATTTACCTCA |
| 1157 | PLXNC1 | 17241948 | 24424385 | 12 | 93146670 | T | A | TACCTAATTCAGGGNTCCATAGCTGAGAA |
| 1158 | PLXNC1 | 10507035 | 24424504 | 12 | 93157454 | G | A | TTGTAGAAATACCCNTTGCCTACCAATAT |
| 1159 | PLXNC1 | 2068435 | 23407167 | 12 | 93163442 | T | C | AATCCTCCAGCATTNCAGTCATGAGACAA |
| 1160 | PLXNC1 | 11107494 | 23985381 | 12 | 93170957 | T | C | TTTACAAAACATTCNTGTACTTCCAAAGG |
| 1161 | PLXNC1 | 832507 | 24424639 | 12 | 93174033 | A | G | TTGCCGATACTATCNTCAAGCCTTTCCCA |
| 1162 | PLXNC1 | 3803070 | 23985436 | 12 | 93191156 | A | C | ATCACAGAGCCCCGNAGCAAGCGCTGAAC |
| 1163 | NY-REN- | 3803071 | 24424763 | 12 | 93201348 | C | T | GATTTTACCACTCANAGCATCAGGTATGC |
| | 58_PLXNC1 | | | | | | | |
| 1164 | NTN4 | 10859918 | 23416268 | 12 | 94549154 | G | T | TTCTGGGCAGAATANTTAACCGATACTGT |
| 1165 | NTN4 | 7315571 | 23416360 | 12 | 94560745 | A | G | AAAAGTTCTGTATCNATGTTGAGTTTGTT |
| 1166 | NTN4 | 17286105 | 24431580 | 12 | 94586791 | G | A | TATGAGAACATTTANGCATTTTTCTCTTA |
| 1167 | NTN4 | 12423171 | 24431652 | 12 | 94593526 | A | G | CATTAATCAATCTGNAATTCCTTTCACCC |
| 1168 | NTN4 | 2431010 | 24431742 | 12 | 94600355 | T | C | CTGAACCTTTAATANGATTCAATAGCTCT |
| 1169 | NTN4 | 2254316 | 24431836 | 12 | 94606606 | T | C | AATGACCTCCTTCTNTTGGGTTAGCAGAA |
| 1170 | NTN4 | 17287370 | 24431942 | 12 | 94613586 | A | G | TTACCTCGTCAAACNTTCACATGTATCAC |
| 1171 | NTN4 | 10777721 | 24431974 | 12 | 94619421 | G | T | AGACTATCTTTATTNATAACTGTGGACTT |
| 1172 | NTN4 | 4128893 | 24432064 | 12 | 94626077 | A | G | ACAAATGAAATACCNTGGAGACACGATTT |
| 1173 | NTN4 | 11108226 | 23417259 | 12 | 94634547 | C | T | ACCTGGGACAGAGCNGCCCTGTTATTCTA |
| 1174 | NTN4 | 4341610 | 24022941 | 12 | 94651756 | A | C | AAGTACCTACTTGTNTTAGAGAAGTGTGT |
| 1175 | NTN4 | 7959421 | 24432438 | 12 | 94678510 | T | C | AAATAAAACTGACANGAGCCTTACCCTCT |
| 1176 | NTN4 | 4762623 | 23417680 | 12 | 94680456 | G | A | GGAGTTGAAACTGCNAAATAAATTTGTAG |
| 1177 | NTN4 | 17289597 | 24432577 | 12 | 94693363 | C | T | CAAACAAATAAAGGNTTTGGCAAGTCAAG |
| 1178 | EFNB2 | 1407584 | 23320878 | 13 | 105937791 | A | C | TACAACTGGCCAGANACTGTGCTGCTATA |
| 1179 | EFNB2 | 9555252 | 23933400 | 13 | 105944377 | C | T | CACCGAACAGACTGNCACCCCAAATGTTA |
| 1180 | EFNB2 | 9301140 | 23321095 | 13 | 105950970 | C | G | AAACGTTGCCCTTANATACAGCTGCACTG |
| 1181 | EFNB2 | 7983579 | 23933497 | 13 | 105957447 | G | T | TTAAATGCTTGAACNCAGCCATTATGCAC |
| 1182 | EFNB2 | 2391333 | 23933551 | 13 | 105964695 | C | T | CACAGTGGGAGCCTNTCTGTGGTATCCCC |
| 1183 | EFNB2 | 2893262 | 24357891 | 13 | 105970807 | A | G | TTTGGGCCTGCCACNTCTATACGGAGCTA |
| 1184 | EFNB2 | • | 46561432 | 13 | 105981057 | T | G | GTCACGCTGGGATCNCGAATAGTTTCCCA |
| 1185 | NFATC4 | • | 46563063 | 14 | 23900690 | T | C | CCTTTATAATATTCNTCTTTTGCTTAAGT |
| 1186 | NFATC4 | 2295298 | 24709564 | 14 | 23913460 | T | C | GAACTGGTACTGACNGGCTCCAACTTCCT |
| 1187 | NFATC4 | 10129213 | 23799265 | 14 | 23926042 | T | G | TTAACCAAAGGTTCNGGGATTCATGCTCC |
| 1188 | CFL2_LOC441 | 17453240 | 24555612 | 14 | 34248919 | G | C | ACTGTTTTTTCTGANTGGCAAACCTAAGA |
| | 681 | | | | | | | |
| 1189 | CFL2_LOC440 | 6571682 | 24555752 | 14 | 34258119 | G | A | TATCAGATTCACCCNCCAGTTATTTGTAA |
| | 176_LOC441681 | | | | | | | |
| 1190 | PAK6 | • | 46552269 | 15 | 38321369 | A | G | GTCAGGAGGAACCCNTAAGTGTCTCTCAT |
| 1191 | PAK6 | 16970474 | 24035934 | 15 | 38342704 | A | C | AAGAGCCAAAAGAGNACATGACGGCCCTG |
| 1192 | PAK6 | 7183540 | 23491750 | 15 | 38347607 | G | A | TTGTGGGAGCAGGCNGTAGTTATCTCGAG |
| 1193 | CHP_MGC336 | 7166182 | 23358004 | 15 | 39301991 | A | G | CACTTTCCTAATTCNAGCACTTCTAGGTA |
| | 37 | | | | | | | |
| 1194 | CHP_MGC336 | 16971693 | 23358053 | 15 | 39308629 | C | T | AATGTTACTGAGTANAAAGGACAACATCA |
| | 37 | | | | | | | |
| 1195 | CHP_MGC336 | 7166556 | 23955512 | 15 | 39314666 | T | C | AGTTTCTGTAATCANCACCCTTAATTCAC |
| | 37 | | | | | | | |
| 1196 | CHP | 2164996 | 23955533 | 15 | 39323916 | T | C | TTGTTTTGTCTTCTNTTATTTACCAACCC |
| 1197 | CHP | 11858115 | 23955540 | 15 | 39329883 | G | A | ACCATTGCCATAGANATTGTTTTTTCTAA |
| 1198 | CHP | 6492998 | 24387112 | 15 | 39333923 | G | A | ACCTCTGTCAGACANGAGGAGCTACATTT |
| 1199 | CHP | 16971734 | 23358257 | 15 | 39349685 | C | T | ATTGTTGGTTTTCCNTGAGTGGTACCTGG |
| 1200 | SEMA6D | 16959955 | 23422868 | 15 | 45789992 | T | C | ATATTGTGGTAGTTNATGAACATAATTCC |
| 1201 | SEMA6D | 17329236 | 24436660 | 15 | 45804781 | A | C | CAAGTGTTTTAATANATCCGCTAATTGAG |
| 1202 | SEMA6D | 628501 | 24436756 | 15 | 45814620 | C | G | TTTCTGATTTTCAGNCCAAGAAGTTTTGT |
| 1203 | SEMA6D | • | 46552230 | 15 | 45822977 | G | A | GTTTATTTGCAGACNTAAAAGGCAGTTCC |
| 1204 | SEMA6D | 586118 | 24436984 | 15 | 45837068 | C | T | GAACCAAATCATTGNGGTTGTTGAGGTAG |
| 1205 | SEMA6D | 483656 | 24437083 | 15 | 45843224 | T | C | AAGACTGCTAGATTNTTTTGAACCAGATG |
| 1206 | SEMA6D | 1344312 | 24437288 | 15 | 45857225 | A | G | ATAAATGACAGTCANTTCAAAAATCAGGG |
| 1207 | SEMA6D | 607289 | 24437460 | 15 | 45862845 | A | G | AAATCCCCTGCTCCNTGTTATGTCTTTTC |
| 1208 | SEMA7A | 7176416 | 23994902 | 15 | 72481543 | T | C | TTTGTACGTGTGTANGGGTGAGCAGGTCA |
| 1209 | SEMA7A | 885740 | 23994960 | 15 | 72492735 | G | A | ACCTCTATTTTTCANTCTAGCCCAAATAC |
| 1210 | SEMA7A | 11852686 | 24437188 | 15 | 72502258 | T | C | GTGCTGTCACCGTCNGGCCCGCTGTCCCC |
| 1211 | SEMA7A | 16968837 | 23423619 | 15 | 72515006 | A | G | TTCAAATGGATTCANAATCAATGATAACA |
| 1212 | SEMA4B | 12438592 | 23533046 | 15 | 88526471 | C | T | CCTAAGGCAAGACCNCTAATTTAGCTGTG |
| 1213 | CIB1_LOC390 | 7167139 | 24059429 | 15 | 88573386 | G | C | GACCAGCTTGGGCTNCGTGCGTTCTGCCT |
| | 637_SEMA4B | | | | | | | |
| 1214 | NFATC3 | • | 46552724 | 16 | 66689610 | T | C | CATCTTTCCCTTTCNCTTGGGTAAATACC |
| 1215 | NFATC3 | • | 46552723 | 16 | 66704360 | G | C | ATTTATGTTCCTCTNTATACTAGCCATTC |
| 1216 | NFATC3 | • | 46552722 | 16 | 66749109 | A | T | TAAACTAATTTGGANATTTGGAGGGCTGC |
| 1217 | NFATC3 | • | 46552721 | 16 | 66783016 | G | A | CAAGCACAAAGTACNGGCCAGGGGGGTCT |
| 1218 | FLJ21918_NF | 8056649 | 23759137 | 16 | 66817584 | C | T | AGTAGTTTGTGGAGNGATGTTTCCATCTC |
| | ATC3 | | | | | | | |
| 1219 | NFAT5 | • | 46552500 | 16 | 68147867 | A | G | GAAAGCCTGCTGGCNCAACTCTTTACAGG |
| 1220 | NFAT5 | 7196943 | 23983928 | 16 | 68149950 | T | A | AATCCTTGGCATAANGTTTAAGATCTTTC |
| 1221 | NFAT5 | • | 46552719 | 16 | 68175014 | C | G | AATCAGTCATAGATNTAAAGCTGCAGTAA |
| 1222 | NFAT5 | 16958965 | 23984026 | 16 | 68181014 | G | C | TAAGTTTTATATTTNACTAAGGTTGAATA |
| 1223 | NFAT5 | 12447326 | 23984050 | 16 | 68188354 | C | T | GATGAGGTGGGAAANAAAGAGGAGTCCCT |
| 1224 | NEAT5 | 39999 | 24423237 | 16 | 68211197 | C | G | TTGGGTGATCAGCTNTTTGGACATAGAGT |
| 1225 | NFAT5 | 244415 | 24423318 | 16 | 68224184 | G | A | CACTAACGAGAAAGNTAATTCACCTACAG |
| 1226 | NFAT5 | 9921045 | 23984322 | 16 | 68266199 | C | T | TGAAAGATATCTAGNACCATTGCTGGTAC |
| 1227 | NFAT5 | 9934681 | 23984364 | 16 | 68276473 | C | T | AGGTACTTATAATANTTTGCAGGGTATCT |
| 1228 | NFAT5 | 17232663 | 24423551 | 16 | 68281966 | G | A | CTAAATGCTAAGAGNAGAGTGTAGTTGT |
| 1229 | NFAT5_NQO1 | 7359387 | 23984452 | 16 | 68291166 | T | G | TTTATATTGTTACANTTAAGTGTTCTTGC |
| 1230 | NFAT5_NQO1 | 1043470 | 24423683 | 16 | 68295409 | C | T | GAACCTCAGGACAANGTCTACACACACAC |
| 1231 | NTN1 | 1880646 | 24210132 | 17 | 8870570 | A | G | GTTCACATGCTTTTNTACACATGTGCATT |
| 1232 | NTN1 | 9891446 | 23787139 | 17 | 8876141 | C | G | GCACTAACCCTGAANAGGGATAGGCGCCC |
| 1233 | NTN1 | 10521156 | 24700632 | 17 | 8883455 | G | A | CAATAGGGCTTTTANGATATCAGAAAAGT |
| 1234 | NTN1 | 16957821 | 23787144 | 17 | 8888829 | C | G | GTAACTCGATCTTCNACAGATTAAGTGAC |
| 1235 | NTN1 | • | 46553733 | 17 | 8900531 | A | T | TCTGTCTTTCTGTTNGTTGGTGGCTAATG |
| 1236 | NTN1 | 8082309 | 24210172 | 17 | 8906581 | G | A | TCTTTGCAGCCAGGNGAACCTTTAGCTAA |
| 1237 | NTN1 | 17808998 | 24210182 | 17 | 8919071 | C | T | CAACATGTTGTGGTNGGGCCTAGTTGGTT |
| 1238 | NTN1 | 869019 | 24700670 | 17 | 8925627 | A | G | CCACACATCCTGCCNTGAAGTACTACCTT |
| 1239 | NTN1 | 17809028 | 24210191 | 17 | 8932957 | C | T | TTCACCCTGCAAAGNGTGATATGTCTGAA |
| 1240 | NTN1 | 4791777 | 23787215 | 17 | 8942135 | A | G | TTCCTACCGTTATCNTGACTGTAGAGTGG |
| 1241 | NTN1 | 9912118 | 23787222 | 17 | 8948930 | C | T | GGACCTGCTGCTCANAGGACATTTCAGAG |
| 1242 | NTN1 | 4791794 | 24700711 | 17 | 8967535 | T | C | AAGTTGCCGTCTATNAGATACGTATTTTA |
| 1243 | NTN1 | 8071818 | 24700718 | 17 | 8979603 | C | T | TTGGCTGAGATCCANGCTTCTTGGAGCCC |
| 1244 | NTN1 | 16957959 | 24119359 | 17 | 8987583 | C | G | AACTGTGGCTCTCTNTCCTGAATTCCATG |
| 1245 | NTN1 | • | 46553732 | 17 | 8998585 | T | C | CAAAAGGAGCCCTTNGAGAGGTTGGCTCA |
| 1246 | NTN1 | 2315287 | 24588860 | 17 | 9005913 | G | C | ACAGGUAACCTGAGNGCAGGATGGTAAGG |
| 1247 | NTN1 | 3785977 | 23631718 | 17 | 9021296 | G | A | CCCTGCCATTACCGNGGACGGCCCTCACT |
| 1248 | NTN1 | 8067387 | 24588995 | 17 | 9028146 | A | G | ATATTTTAGGATACNGACTGGATTTTGGA |
| 1249 | NTN1 | 12945811 | 24119660 | 17 | 9035059 | C | T | TATGCTGTATATCANCAATGGATTAAAGA |
| 1250 | NTN1 | 12450397 | 24589094 | 17 | 9040765 | A | G | GAGAAGAGTGTGACNAGTGTCATTTGCAC |
| 1251 | NTN1 | 7212084 | 23632078 | 17 | 9046448 | C | T | TGTGAGGGCAGCTGNGTCTCTGGCTGTGT |
| 1252 | NTN1 | 10852913 | 24589321 | 17 | 9058298 | G | T | GGCTTGTATAGTTTNCGCTTCTTGGGATT |
| 1253 | NTN1 | 3785988 | 23632323 | 17 | 9065630 | G | A | GCATCATACTGCGGNGATTTGACCCGAGT |
| 1254 | ROCK1 | 7244299 | 23800139 | 18 | 16810503 | C | T | TCTTCAGTTTGTTANAATCCTTACAACTT |
| 1255 | ROCK1 | 8085654 | 23800143 | 18 | 16824233 | G | A | TAGCAGATGAATTANCAGATGATGAGGTC |
| 1256 | ROCK1 | 17202347 | 24710275 | 18 | 16849138 | G | A | ACATTTCAACCATCNAGAACCACTGAACT |
| 1257 | ROCK1 | 7239317 | 24710279 | 18 | 16864065 | C | T | TTAGGGCTTTTCCANCATTCCACTTTCCT |
| 1258 | ROCK1 | 11876799 | 23487572 | 18 | 16927597 | C | A | AAGTTCTCATGTGANCCTACCCGCCTGAT |
| 1259 | DCC | 16954702 | 23772067 | 18 | 48113019 | T | C | ACACTTCATTATAGNTTGGATCATATTTA |
| 1260 | DCC | 16954731 | 23772114 | 18 | 48126639 | C | G | AAAGTATTATGCACNTTTATCATTGCAGG |
| 1261 | DCC | 10502942 | 23772159 | 18 | 48132806 | C | T | TTGATTTAGAGGCTNATAGGTCCAGCTAA |
| 1262 | DCC | 17669415 | 24689429 | 18 | 48138995 | C | G | CAATATGAACATAANCAAAATTACTCCTC |
| 1263 | DCC | 9965892 | 24201074 | 18 | 48145226 | G | A | CAGTCCTTGGTCATNAGACTGAATGAGTT |
| 1264 | DCC | 3910783 | 24201084 | 18 | 48155562 | G | T | TAGAGACAAAAAATNAGAAGTCAGTGACT |
| 1265 | DCC | 12960169 | 23772396 | 18 | 48178779 | T | C | AGGTGTTACTAACTNGATCATTCTTCTGG |
| 1266 | DCC | 3915401 | 24689569 | 18 | 48188604 | C | A | TAGCTGTCAAAATCNAGATTTTTTTAAGA |
| 1267 | DCC | 9956738 | 23772468 | 18 | 48194971 | A | G | TGTGCATTAACTGCNTAGACAGCTGTGGA |
| 1268 | DCC | 17748116 | 24689626 | 18 | 48203972 | C | A | AGGGAATGAGAGAGNAAATTCTAGTCAAA |
| 1269 | DCC | 2339345 | 24201240 | 18 | 48211253 | A | C | TCACTAGACACTTTNAATTACTGTCTATA |
| 1270 | DCC | 1443581 | 24201272 | 18 | 48218841 | G | A | TTCACATTAACATANTCTACTTAGCTCAT |
| 1271 | DCC | 11082925 | 23772612 | 18 | 48230717 | T | G | CATACCTTTCCAGTNTATTCAGTAAATGC |
| 1272 | DCC | 2119839 | 24201305 | 18 | 48244707 | A | G | ACATCAGTGCTTAGNATATAAAGTATGTG |
| 1273 | DCC | 16955062 | 23772720 | 18 | 48257594 | T | C | ACTTGACTGTGGGANCACCATTCCCTGCA |
| 1274 | DCC | • | 46553820 | 18 | 48280140 | C | G | TCCCTTTTTAATGANTACCCACACAGAGC |
| 1275 | DCC | 17828227 | 24201390 | 18 | 48288557 | C | G | GAGAAGAAAGATGTNTGCTTTTCTGTTAT |
| 1276 | DCC | 16955130 | 23772855 | 18 | 48294683 | T | G | TTGCTGCTTAGACANGGTTTTTTTTATTG |
| 1277 | DCC | 1145261 | 23772872 | 18 | 48303362 | T | C | AATTATTCTGGTAANATTGGTATGAAGTG |
| 1278 | DCC | 7240443 | 24201430 | 18 | 48331798 | C | A | TCAGTGACGCTCTANCATAGCTTGTTATA |
| 1279 | DCC | 10502948 | 24689822 | 18 | 48339348 | G | T | TCTCATGAATAATTNCAGAACTAGGATTA |
| 1280 | DCC | 948593 | 23772940 | 18 | 48347247 | T | A | ATACATTTGACAACNATATGACGTACCAT |
| 1281 | DCC | 9946542 | 24201458 | 18 | 48354022 | A | G | TGCTGTCTCTTTCTNTTTCACTGGTTGAC |
| 1282 | DCC | 12961437 | 23772992 | 18 | 48360097 | T | G | AAGGAACGTTAGGANGGACCTATCTGGAA |
| 1283 | DCC | 1563225 | 24201483 | 18 | 48371396 | A | C | GTTATATTATCAAANACATTGAAGTTTTA |
| 1284 | DCC | 12456699 | 24201500 | 18 | 48378244 | A | G | GTAGAGAACAGTGANGATATTCTAGAGTT |
| 1285 | DCC | 17681706 | 24689899 | 18 | 48385281 | A | G | AGTTGGTATGCTTCNAAGTGATTAGCTCA |
| 1286 | DCC | 1221862 | 23773076 | 18 | 48397757 | T | C | TATTGAATGCTTAANGTTAATGGCTGTGA |
| 1287 | DCC | 8097187 | 24201560 | 18 | 48407778 | A | T | GAGATTCACAAGACNTCAACACTGCCTTC |
| 1288 | DCC | 17681801 | 24689931 | 18 | 48427619 | C | T | TGTTGACTTTGGTCNTCACTCCTTTAGGC |
| 1289 | DCC | 11877045 | 24201603 | 18 | 48439992 | G | A | TGCAGATGCCCTGANCTTTGGAATGAATT |
| 1290 | DCC | 17828903 | 24201620 | 18 | 48447955 | A | G | AATAAGAACAATTTNTCCATGACAGCTTT |
| 1291 | DCC | • | 46553890 | 18 | 48454103 | A | G | CAGGTTTAGATCACNTCGCTAATGTTCTT |
| 1292 | DCC | 12962177 | 24690039 | 18 | 48464878 | C | T | CTTTGTGTGGTGATNGTACACTCTCTGCA |
| 1293 | DCC | 17829061 | 24201672 | 18 | 48471391 | C | T | GCAAAGTGCATAGANGGATTTATTAATTT |
| 1294 | DCC | 16960544 | 23773219 | 18 | 48478896 | C | T | ACTGUCAATTCTTGNTCCCCATTGTTCTA |
| 1295 | DCC | 1160400 | 24690115 | 18 | 48485283 | A | G | GTTGCAGCCTGGCCNTTTACGTTCTGGCC |
| 1296 | DCC | 17756182 | 24201727 | 18 | 48491407 | G | A | CAATAACTTTATCCNGCTGATGAGCACAT |
| 1297 | DCC | 4641847 | 23773306 | 18 | 48503880 | C | A | GGTTTGGCTTCAGCNGTCCCTCAGACCTA |
| 1298 | DCC | 17468382 | 24546575 | 18 | 48515009 | T | C | CTAGCAATCCAAGCNTTAGACCCAGTGGA |
| 1299 | DCC | 12953702 | 24546647 | 18 | 48524257 | A | G | ACCCTTTTGCTGAGNATATTTGGTGGTGT |
| 1300 | DCC | 17469012 | 24546753 | 18 | 48550456 | A | T | CCACTGCTTTTAGGNTCAAATATAAACCT |
| 1301 | DCC | 11082944 | 24546824 | 18 | 48556392 | C | A | CACTCACACTACAGNAGAGTTGGATACTG |
| 1302 | DCC | 1821121 | 24546896 | 18 | 48563063 | C | T | GTGACTGGCTGAACNTGGATCAATTGCTC |
| 1303 | DCC | 17470011 | 24547043 | 18 | 48589648 | T | C | AACTAGAGATACCANGTATTTTGTTACTG |
| 1304 | DCC | 16955568 | 23577430 | 18 | 48599026 | C | A | AGATCAATGGTCACNAAACGTTTTATTTA |
| 1305 | DCC | 10502951 | 24547443 | 18 | 48620838 | T | C | AGTAACACCAATATNTGTAGAACCATTCT |
| 1306 | DCC | 8088779 | 23577999 | 18 | 48627805 | G | T | CAAGGGCTCAGAACNGTAGCTATCTGATC |
| 1307 | DCC | 17478731 | 24547714 | 18 | 48638468 | G | A | GAAGTTTAGATAGCNAGAATCATGCATTG |
| 1308 | DCC | 1943098 | 24547785 | 18 | 48658375 | G | T | CCTGTGTGTCTCTGNGGCTAAATATAAAC |
| 1309 | DCC | 17479650 | 24547978 | 18 | 48672713 | A | G | AGATAGTGCCCCCANAGAAGCAGGGTAGA |
| 1310 | DCC | 1943108 | 24087221 | 18 | 48679020 | T | C | ACTTTTGATGTCCCNACAGATTTAGCAGC |
| 1311 | DCC | 17389547 | 24548275 | 18 | 48686704 | A | C | TTATCAGGTATTGCNATGCTCTTTGTTGC |
| 1312 | DCC | 9964756 | 23579301 | 18 | 48695815 | T | G | TATGCCCCTCAGTANGTCACACACACTCA |
| 1313 | DCC | 1943126 | 24548567 | 18 | 48704488 | T | C | TTGAGATGGTCCTGNCCAAAGAGTCTTCA |
| 1314 | DCC | 4564688 | 24548839 | 18 | 48712944 | C | A | ATCTTTGAACTTTCNAGAAATGGTTCTAG |
| 1315 | DCC | 12456218 | 24087962 | 18 | 48724069 | T | A | TTCTGAGTTTATGTNGTAAATTGTAATTT |
| 1316 | DCC | 4147355 | 24549040 | 18 | 48732362 | G | A | TGCACATTTGCTGGNTCTATAGATGTAGA |
| 1317 | DCC | • | 46554206 | 18 | 48738994 | G | A | GAAAATGGAGACACNGAAACACATCAGAG |
| 1318 | DCC | 16955910 | 24088096 | 18 | 48739551 | T | G | AATAGGTCAAGAATNGAATCGTGTGAATG |
| 1319 | DCC | 7238466 | 24071431 | 18 | 48777957 | A | G | AGCCATTTTCTGCCNCGATTGTGGATTTG |
| 1320 | DCC | 7506454 | 23553651 | 18 | 48787669 | G | A | TATAGTAAATTTACNGTAGGTTTAGACGT |
| 1321 | DCC | 8088299 | 24071618 | 18 | 48810723 | T | G | TCTGCCACATAATANATACTCAATAAAGT |
| 1322 | DCC | • | 46554205 | 18 | 48842943 | T | A | TAGGCAAGGTTTTGNGGGCTAAGCCTGAG |
| 1323 | DCC | 8082863 | 23554045 | 18 | 48848566 | C | G | CTGAACTAGTAATGNATAAGTAGGAGAAA |
| 1324 | DCC | 4291980 | 24549109 | 18 | 48871173 | G | T | AAGCTTAATAATTTNTCAAAGTTCAACTA |
| 1325 | DCC | 9961246 | 24088277 | 18 | 48881563 | C | A | AGGTGGCCCAAGGTNGATTGTGAACACTC |
| 1326 | DCC | 16956165 | 24088376 | 18 | 48893969 | A | G | TTTAGTCACATGGANATAAAAGTTGACCT |
| 1327 | DCC | 9957822 | 24088421 | 18 | 48901321 | T | G | AAGACTATGATGATNAATGGAATTATTAC |
| 1328 | DCC | • | 46554204 | 18 | 48911104 | C | A | CCAACCATGTCTTANCTTAGTGTCCTTAG |
| 1329 | DCC | • | 46553825 | 18 | 48918767 | C | T | TTATCTTATGGTTGNCTACTATTCATAAA |
| 1330 | DCC | 8083973 | 24549710 | 18 | 48931254 | A | G | CTCAGAGACCCTCANCTGTATCTTTCAGG |
| 1331 | DCC | 2276147 | 24088707 | 18 | 48937689 | G | A | TTACCTATGGCAGCNACACCTCTGATAGC |
| 1332 | DCC | 4940245 | 24088788 | 18 | 48948135 | A | G | ATACAAAGTGATACNTAATACACTAGATG |
| 1333 | DCC | 17408063 | 24549941 | 18 | 48958837 | C | G | ACTCAGGCTCCATANATAGAACACTAAGC |
| 1334 | DCC | 12959099 | 24550066 | 18 | 48965508 | A | G | AAAAAAATGTTTTCNGGTGTTATAAATAA |
| 1335 | DCC | • | 46554208 | 18 | 48981718 | A | G | AAGGAACACATTCANTGAGTACTTTACTT |
| 1336 | DCC | 12963433 | 24089129 | 18 | 48987691 | A | C | TTTCTTGGTGAATTNTTCCATTTTGGGTA |
| 1337 | DCC | 4609919 | 23582002 | 18 | 48998837 | A | G | ACATCTAGATAATGNAACTAAGAACAAAA |
| 1338 | DCC | 1367638 | 24550493 | 18 | 49005423 | G | C | GATAAAAAGAGGAANGGTGATTTCACAGA |
| 1339 | DCC | 16956392 | 23582216 | 18 | 49011380 | T | C | TTCTGAGACTATTTNCCAGGAATGTAGTT |
| 1340 | DCC | 2339638 | 24089387 | 18 | 49032213 | T | G | TTGAATTAGGTGAANAAATGCATCCTCCC |
| 1341 | DCC | 17411061 | 24550800 | 18 | 49049111 | C | T | TTACTTCTGTTTTGNTCTCTCTCAGTCTT |
| 1342 | DCC | • | 46553889 | 18 | 49055644 | C | A | CTATTACTGCTTTGNAAGTACTAAGAAGA |
| 1343 | DCC | 12709769 | 24089553 | 18 | 49061166 | T | C | AAAACCCGAAAAGGNTGGACTAGATGAGG |
| 1344 | DCC | 1813854 | 23582832 | 18 | 49076958 | G | A | AGTTAGCTATGGCANTATTCTGGGCAATA |
| 1345 | DCC | • | 46554207 | 18 | 49086100 | A | G | AGGTCAGTGTTCACNTGGTGTAGTCTTGC |
| 1346 | DCC | 1367637 | 24089811 | 18 | 49114720 | T | C | ACTTGCCTTGCCTANAGTAACCTGGTCTG |
| 1347 | DCC | 16956615 | 23583229 | 18 | 49121844 | A | C | TCTAAAGAGCTAGGNATATTTTTAAAGGT |
| 1348 | DCC | 17503887 | 24551288 | 18 | 49141959 | G | A | TACCTCAGCTATCCNAAAGAACTGTGTCA |
| 1349 | DCC | 11082981 | 23583406 | 18 | 49147352 | C | G | CTGTTTTATTTAAGNTGCTGCATTTTGGT |
| 1350 | DCC | 16956637 | 23583432 | 18 | 49153132 | A | G | TGTGTCTAATCCCANTTCCACGGCCTTCC |
| 1351 | DCC | 17504520 | 24551471 | 18 | 49159355 | G | T | TGCATTATACAAATNGCATCTTTCTCAAA |
| 1352 | DCC | 10163659 | 24551783 | 18 | 49173432 | C | T | TAGGTCTCCTTATANTTTAGCCAATGTTC |
| 1353 | DCC | 16956678 | 24090399 | 18 | 49180702 | T | C | ATGTGTTAAAGAGANACTTATGATTGGCT |
| 1354 | DCC | 1010360 | 24090440 | 18 | 49182439 | T | G | TAGCTAAGATATTTNGACCCCAGTTTTAT |
| 1355 | DCC | 953122 | 24552242 | 18 | 49192693 | C | G | AATAAGTTITTGATNATTTAAAATTACTC |
| 1356 | DCC | 11872591 | 24552358 | 18 | 49202829 | G | A | ACTGGAGAATCTCTNCTCTTGACGTTGTG |
| 1357 | DCC | 8095430 | 24090737 | 18 | 49218895 | A | T | TATATATGCACCCCNAGGTATACATGAAT |
| 1358 | DCC | 12604940 | 24552431 | 18 | 49226213 | A | G | TTATTACTAATTTTNATGGAACACATCTA |
| 1359 | DCC | 10502978 | 23584660 | 18 | 49235438 | C | A | GCAGAGAAATGTTTNTACTTATCAATATA |
| 1360 | DCC | 9955474 | 23584810 | 18 | 49258268 | T | C | CCCAGCCTAGTTACNCTTACCATAAAATG |
| 1361 | DCC | 4510099 | 24090922 | 18 | 49269568 | C | G | ACTTTATCTAAAATNAAAAGGCATTGGAT |
| 1362 | DCC | 870507 | 24090988 | 18 | 49281972 | A | G | AGAGGACCAATTTGNTAGCAACAAAAAGT |
| 1363 | DCC | 8086556 | 24091046 | 18 | 49290854 | T | C | TCATTTATTGGGTANAAAAATGCCGTAAG |
| 1364 | DCC | • | 46554203 | 18 | 49300004 | G | A | CACAGCTAGGAGCCNTGGAAGAGATTCTT |
| 1365 | DCC | 1502219 | 24091111 | 18 | 49308887 | G | A | TTTTTACATTAAAGNTTGACTCTAAAGAA |
| 1366 | DCC | 16956878 | 23585172 | 18 | 49315397 | T | C | AAGGGATCTCACTGNGTGCTGAACATGTA |
| 1367 | NFATC1 | 7242726 | 24118592 | 18 | 75310556 | G | C | GTTCCCGAGAGATTNATGTGGTACTGTGG |
| 1368 | LOC440503_L | 11878179 | 23570997 | 19 | 4484664 | G | T | AATATACATGAAACNGTTGGAACTGTGAC |
| | RG1_SEMA6B | | | | | | | |
| 1369 | SEMA6B | 1610093 | 23571021 | 19 | 4505774 | T | C | GTTCCATTTTGGGCNTCTAAGCCACCCAC |
| 1370 | PAK4 | 17795295 | 24195905 | 19 | 44323525 | C | T | ATTCTTTGCCATTANGATGAGGTCACATT |
| 1371 | PAK4 | 513053 | 24683659 | 19 | 44332986 | A | G | CCTCACAGTTGCCCNAACAGCGCTGCCTC |
| 1372 | PAK4 | 17641276 | 24683662 | 19 | 44365874 | A | G | ATAAAAACAGATCCNGCTTAGTCAAGTGT |
| 1373 | C20orf103_PA | • | 46555546 | 20 | 9459363 | A | G | TTTGGCAAATATAAANTAGTTTAATAATG |
| | K7 | | | | | | | |
| 1374 | PAK7 | 2072952 | 24088446 | 20 | 9473601 | T | C | TATGTACCATGAGGNGAATCAAAGAACCA |
| 1375 | PAK7 | 10485733 | 24088511 | 20 | 9492722 | T | C | ATGTGGATTGTACANGCTTTCCTGGAGAC |
| 1376 | PAK7 | 17406954 | 24549628 | 20 | 9500795 | A | G | TAGTCATCACAGCANAACGAGTTGATTAT |
| 1377 | PAK7 | 11696945 | 24088687 | 20 | 9520689 | T | A | TGCCAAGAAAAGAGNATGCCTTATGCAAA |
| 1378 | PAK7 | 4816152 | 24088755 | 20 | 9531794 | G | A | TTATAGACGCCTCCNGTGTAGAAAATGGC |
| 1379 | PAK7 | 6056750 | 24088812 | 20 | 9539204 | C | T | GGATGTACCTGATGNTTGAATTAACAAAT |
| 1380 | PAK7 | 219861 | 24549927 | 20 | 9545555 | A | G | TTTCTTCTGGGAAANTCTTTTTGTTTATT |
| 1381 | PAK7 | 219870 | 24549949 | 20 | 9550875 | G | T | TGAGGTAATGCTAGNCAAGACTTTATCCA |
| 1382 | PAK7 | 6056767 | 24549991 | 20 | 9561645 | G | A | CTTACCCAAAAGACNGAGTTACTACTCTC |
| 1383 | PAK7 | 6056774 | 23581558 | 20 | 9572587 | C | T | AAAGCCAAAGTTCCNTTTCATGGGCTGCA |
| 1384 | PAK7 | 1885562 | 24550104 | 20 | 9577963 | G | A | TAGTTTAGTTCCTANAGAAG0ATAGTAAA |
| 1385 | PAK7 | 6086976 | 24550153 | 20 | 9584296 | T | C | AGATGTATCTATCANTCTGCATAAATACG |
| 1386 | PAK7 | 2423395 | 24550197 | 20 | 9593420 | G | A | TCATGTTACATATTNTAGCCTCATAAATA |
| 1387 | PAK7 | 2423412 | 23581905 | 20 | 9603984 | A | G | ACAATAAAATAAGGNTCATACAGCTTGGG |
| 1388 | PAK7 | 2423422 | 24089164 | 20 | 9620074 | A | C | TCACGAAAATCATANGACTCTACGGAAAG |
| 1389 | PAK7 | 2876169 | 24089201 | 20 | 9624293 | T | G | CACATTAATTAAGANGTGAAGGTTGGCAA |
| 1390 | PAK7 | 2206474 | 24089229 | 20 | 9631016 | A | G | TGCATGCTTGCATCNATACTCAAATATTA |
| 1391 | PAK7 | 6039556 | 23582184 | 20 | 9637896 | T | A | TTCATCCTGACTCANTTCTCTAAGTATAT |
| 1392 | PAK7 | 6056820 | 23582238 | 20 | 9643213 | C | T | CCAATTAAGTTTACNTCTCAATATTTTAA |
| 1393 | PAK7 | 2423438 | 24550592 | 20 | 9652943 | T | G | CTCTCAAGTGTAAGNTTTCAAGGCACTCG |
| 1394 | PAK7 | 2206486 | 23582389 | 20 | 9662982 | G | C | GGATGGCCTCCATGNAAGAGTAAAATGAA |
| 1395 | PAK7 | 2423460 | 24089430 | 20 | 9674101 | T | C | AAAGGAAAAAATCCNTTACAGCTATTAAA |
| 1396 | PAK7 | 6118715 | 24550749 | 20 | 9680540 | C | T | TCTGCTCTGTAAGANATCAACAAACATTT |
| 1397 | PAK7 | 16996384 | 24089514 | 20 | 9687730 | A | G | TTACTGAGAAAGCANGCAAGCATTCAATA |
| 1398 | PAK7 | 2327225 | 24550887 | 20 | 9696128 | C | A | ATGTGTCTGAAATTNTATTATTTGTCTAT |
| 1399 | PAK7 | 6056861 | 24550963 | 20 | 9702779 | T | C | AATCTGGGACTACCNAGGGTGCTGAAGAC |
| 1400 | PAK7 | 6056867 | 23582886 | 20 | 9708229 | A | C | TTAACTGATTATAANTTTTCTGTCAGTTT |
| 1401 | PAK7 | 6056871 | 24551019 | 20 | 9713652 | T | C | TCCAATAATTTCAGNAAGCCGTCATATTA |
| 1402 | PAK7 | 10485735 | 24551067 | 20 | 9721877 | G | A | GCTAACTTCCTGGANATATGAACTATATT |
| 1403 | PAK7 | 16996420 | 24089754 | 20 | 9728868 | C | T | TTTGAGATTCATGANGCTTTGAGGATTCT |
| 1404 | PAK7 | • | 46555551 | 20 | 9731166 | T | G | ATCTGTTGGAGGCANTAACTTCTTACATA |
| 1405 | PAK7 | 2050107 | 24551240 | 20 | 9744339 | C | T | GATTAGTGGTCTACNATAGCAATTCTTTT |
| 1406 | PAK7 | 7263675 | 23583322 | 20 | 9751165 | T | C | CTTTGAGGGCAACTNAACACTCTGGCTCA |
| 1407 | PAK7 | • | 46555074 | 20 | 9757383 | T | C | AAGAATTTCTTAAANAGTATGCCAAACTT |
| 1408 | PAK7 | • | 46555550 | 20 | 9764861 | A | C | TAATGTTGTCATCANACACAGTTCATAGG |
| 1409 | PAK7 | 761226 | 24551431 | 20 | 9769007 | A | G | ATATTGTACTGGCCNTATCAAAACAGAAA |
| 1410 | NFATC2 | • | 46555248 | 20 | 49440789 | C | T | TGGCATGGACTGGGNGAGCTCTAAGGACT |
| 1411 | NFATC2 | • | 46555247 | 20 | 49447237 | C | A | GTGTTCATCTTCTCNAGGGAAACCTGGCC |
| 1412 | NFATC2 | 6067766 | 23784308 | 20 | 49473493 | A | G | CTTTAAAATTGGTTNTGATGCTGACCTAC |
| 1413 | NFATC2 | 2244571 | 24208216 | 20 | 49482966 | G | A | TGACTCAGCCCTGGNATGTTTACAGCCTA |
| 1414 | NFATC2 | 17199672 | 24208224 | 20 | 49490593 | T | A | CTTTTGCACCCTTTNGGTACCAGAAGCTC |
| 1415 | NFATC2 | 228840 | 23784342 | 20 | 49504665 | A | G | GAAGTCACTGTGATNAGGGGAGATGTCTG |
| 1416 | NFATC2 | 8121649 | 23784355 | 20 | 49514051 | C | T | CTCTCCTCTCACACNGGCCTACTGCTGCC |
| 1417 | NFATC2 | 6021228 | 24698402 | 20 | 49522974 | A | G | TGTGTTAATGTATANGTAGACTTTAAACT |
| 1418 | NFATC2 | • | 46555246 | 20 | 49531477 | T | C | AATTTCCCAGGCAGNTGAAAAGGTTCCAG |
| 1419 | NFATC2 | 6063655 | 24698423 | 20 | 49537488 | G | A | TGCAGTTCAGCTTTNAAAAAATGTTCCAC |
| 1420 | NFATC2 | 3787192 | 24698436 | 20 | 49543965 | T | G | GACGATATAATGGGNACAGTGCCTGGCAA |
| 1421 | NFATC2 | 12481623 | 24698453 | 20 | 49564272 | A | C | CTCCTTAAGACTTANAATTGTTTGGCCAA |
| 1422 | NFATC2 | 6063664 | 24208311 | 20 | 49575231 | C | T | TGACCTAACTTCCTNTTTTAGCGGCTGCC |
| 1423 | NFATC2 | 13039818 | 24698487 | 20 | 49578909 | T | G | TGTAAGGCCTGGAGNTCTTGTGAGGCTGT |
| 1424 | LOC440806_M | 17759568 | 24703202 | 22 | 20435181 | A | G | AGGATATCGGTGATNAAAGTATTTGAAAA |
| | APK1 | | | | | | | |
| 1425 | LOC440806_M | 13943 | 24703209 | 22 | 20440756 | C | G | TTTAGAAGAGGACTNCAAATTAACCTTAT |
| | APK1 | | | | | | | |
| 1426 | LOC440806_M | 17759598 | 24703216 | 22 | 20446703 | T | C | CAGTCCTCAGCTCANGTCTACTTGGTTCT |
| | APK1 | | | | | | | |
| 1427 | MAPK1 | 2283792 | 24703227 | 22 | 20455679 | T | G | TCAGTAACTACCGTNCTACAAAAACTCAA |
| 1428 | MAPK1 | 5749998 | 24703255 | 22 | 20486928 | A | T | GTTAACTGGGTGAANGTCTTGCCTTTATA |
| 1429 | MAPK1 | 2266969 | 24703261 | 22 | 20498461 | G | A | TGCCATAAGGAACANGCTGTTAGCCAGAA |
| 1430 | MAPK1 | 2283793 | 24703271 | 22 | 20506032 | T | C | AAGTATGAAAAACANTTCTCTCTTCATGA |
| 1431 | MAPK1 | 5999752 | 24703277 | 22 | 20512737 | G | A | TTCTTATTCTTTTTNTTGATAACTGTAAA |
| 1432 | MAPK1 | 17759796 | 24703278 | 22 | 20514717 | C | A | GAGAGATCCCTTCANTCTGGAAAGCTGAC |
| 1433 | MAPK1 | 5999842 | 24703282 | 22 | 20539443 | G | A | CTTCTGTTACTCATNTAAGCTGCTGTCCT |
| 1434 | FLJ38628_LLM | 5749231 | 24212313 | 22 | 29923642 | A | C | AAGGCTAACCTTGGNTTCCCCAGCTAAGC |
| | K2 | | | | | | | |
| 1435 | LIMK2 | 5997919 | 24703552 | 22 | 29947996 | A | G | AGAGTTGGAAAGCANCCATCATTATTATC |
| 1436 | LIMK2 | 5753518 | 24212323 | 22 | 29955868 | G | A | GTAAATGGCAAGACNGAATCAGTTCCGGA |
| 1437 | LIMK2 | 2106294 | 24703576 | 22 | 29970313 | C | T | GCAGAAAAGCAGTGNACCATGTGGTTTTG |
| 1438 | LIMK2 | 2413047 | 24703602 | 22 | 29990580 | A | G | GATGGGGGATAGCCNTGATTGTTGAGCAA |
| 1439 | LIMK2_MGC1 | 16989469 | 23790680 | 22 | 29993348 | C | T | GTGAAGCGTAGGACNGGATACCCAGAGCT |
| | 7330 | | | | | | | |
| 1440 | LIMK2_MGC1 | 1034587 | 24212338 | 22 | 30004794 | G | T | TGAGTTTAACTTCCNCGCCTTGCTTTCCT |
| | 7330 | | | | | | | |
| 1441 | RAC2_SSTR3 | 5756562 | 24601586 | 22 | 35936307 | T | C | GGTTCTTTCCAATANCTCCTTTTGGAGCT |
| 1442 | RAC2 | 2284038 | 24129595 | 22 | 35959555 | A | G | CAGAAGCTCTCTCCNCCTTAATCTCAGGT |
| 1443 | RAC2 | 739043 | 24601614 | 22 | 35969730 | A | G | TGCTGTGAACATCCNTGTGCTCCTGGGCT |
| 1444 | EFNB1 | 16990746 | 23817627 | X | 67840631 | T | C | GTGACAGGTTAGGANACAGATGAGGCCCT |
| 1445 | EFNB1 | 241395 | 24722489 | X | 67852756 | G | C | TAGAGTTGCAGGCANTTTTGAAAGAAGGA |
| 1446 | PAK3 | • | 46556523 | X | 110139832 | G | A | CATAAATTGTCTGANAGGATCTCCACCAT |
| 1447 | PAK3 | 5985318 | 23828071 | X | 110155224 | G | A | TTTCACTGAAAACCNTCACTCTACCCTCT |
| 1448 | PAK3 | 5942721 | 23828087 | X | 110170703 | A | G | GCGATCTCCCGTTCNAATCAAAATCAAAC |
| 1449 | PAK3 | 5943137 | 23828088 | X | 110177597 | T | C | GATATGCAGTGCTGNGTAATGGACTGTGT |
| 1450 | PAK3 | 16986378 | 23828092 | X | 110186485 | A | C | CTGGGAGCCAAAGTNCTCAATGGAGAACA |
| 1451 | PAK3 | 1884552 | 23828100 | X | 110209121 | T | A | TAATAGCCCAGTTGNTATGGCTAAATTAA |
| 1452 | PAK3 | 16986393 | 23828104 | X | 110219841 | A | C | CTAAATTATGTTGTNTACTAGCCCAGAGA |
| 1453 | PAK3 | 10521534 | 23828112 | X | 110227586 | A | G | CACTGTCATCTCTCNTATGAATCACTCTT |
| 1454 | PAK3 | 16986417 | 23828116 | X | 110237476 | A | G | AGTAATTTGTCGCANTTTTATACCACTGG |
| 1455 | PAK3 | 16986424 | 23828117 | X | 110249921 | C | T | CGTTTGTGGGTTAGNCCTCCATGGATTTA |
| 1456 | PAK3 | 7884588 | 23828119 | X | 110259372 | T | C | AATTCAGTTGCACANCAGTGTCAGTGGAA |
| 1457 | PAK3 | 16986440 | 23820390 | X | 110269647 | C | A | AATATATCTTAATTNAAAAGGGCTGCTTG |
| 1458 | PLXNB3_STK | 22668822 | 3831984 | X | 152558655 | G | A | CAGGAAGGACAGGCNCCTAGTAAGGATGT |
| | 23 | | | | | | | |
| 1459 | PLXNA3 | • | 46555937 | X | 153257515 | C | T | ACCGAGCTGGACTTNTCCGTGGTCTGGGA |
| 1460 | DXS9879E_PL | • | 46555936 | X | 153265420 | A | G | GGGGGCCCTAGAACNCTCCTCCTCCTGAG |
| | XNA3 | | | | | | | |

* Always on + strand.

Validation with a Second Whole-Genome Association Dataset

A second available whole-genome association dataset for PD was mined to determine whether the genes in each of the predictive genetic models in the primary whole-genome association dataset were also predictive of the same PD outcomes in the secondary dataset (same genes and outcomes, different SNPs and samples). The secondary whole-genome association dataset included 1,195 SNPs in the 22 genes from the model predicting PD susceptibility in the primary dataset. Of those SNPs, 127 (10.6%) were individually associated with susceptibility to PD.

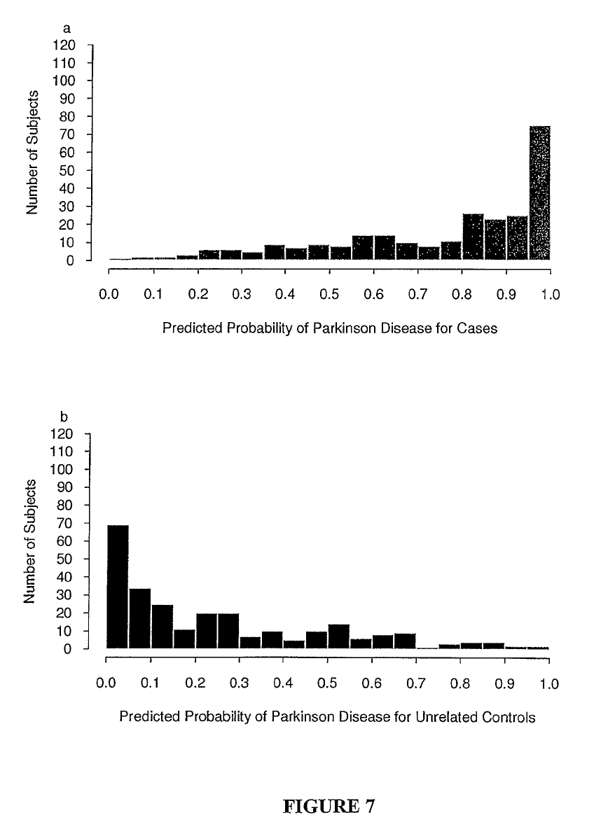

Table 5 contains results for the final model produced by running SNPs through the multi-stage process to predict PD susceptibility. This model used data from 528 subjects (264 PD patients and 264 unrelated controls; 8 subjects were missing data on one or more SNPs). The model had an overall p value of 3.93×10⁻⁴⁴. The odds ratios (95% CIs) for the groups defined by predicted PD probability of <0.25, 0.25-0.50, 0.50-0.75, and >0.75 were as follows: 1 (reference), 7.86 (3.94-15.71), 16.14 (8.13-32.05), and 121.14 (56.63-259.14), respectively. The predicted probabilities of PD were high (towards 1) for most of the cases, and low (towards 0) for most of the unaffected siblings (FIG. 7). Indeed, 38% of the cases had predicted probabilities above 0.9, and 39% of unaffected siblings had predicted probabilities below 0.1. However, as shown by FIG. 7, the model did not completely distinguish the two groups; some cases had low predicted probabilities, and some controls had high predicted probabilities.

TABLE 5
Axon-guidance SNPs predicting Parkinson's disease susceptibility, within a secondary
whole-genome association dataset.

| Gene* (rs ID), | Odds Ratio | | | | Odds Ratio | | |
|---|---|---|---|---|---|---|---|
| Final Coding Scheme^{†} | (95% CI)** | | P value** | Interaction* | (95% CI)** | | P value** |
| CDC42 (rs2056974), d | 2.09 | (1.24-3.52) | 5.6 × 10⁻⁰³ | DCC₅* FYN₁ | 17.01 | (3.06-94.59) | 1.2 × 10⁻⁰³ |
| DCC₁ (rs1160400), r | 7.75 | (1.06-57.01) | 4.4 × 10⁻⁰² | EPHB1₁* EPHB1₄ | 3.82 | (1.39-10.48) | 9.3 × 10⁻⁰³ |
| DCC₂ (rs1466373), d | 2.03 | (1.14-3.64) | 1.7 × 10⁻⁰² | PLXNC1₁* PLXNA2₂ | 29.53 | (1.82-478.49) | 1.7 × 10⁻⁰² |
| DCC₃ (rs1502219), a | 2.42 | (1.55-3.79) | 1.0 × 10⁻⁰⁴ | RRAS2* SEMA5A₂ | 4.85 | (1.44-16.33) | 1.1 × 10⁻⁰² |
| DCC₄ (rs4334396), r | 14.33 | (3.92-52.34) | 5.6 × 10⁻⁰⁵ | SLIT3₃* SEMA5A₂ | 15.93 | (2.00-127.05) | 9.0 × 10⁻⁰³ |
| DCC₅ (rs7506794), a | 0.07 | (0.03-0.19) | 1.6 × 10⁻⁰⁷ | RRAS2* EFNA5₃ | 0.03 | (0.001-0.67) | 2.7 × 10⁻⁰² |
| EFNA5₁ (rs152580), r | 2.78 | (1.49-5.19) | 1.3 × 10⁻⁰³ | | | | |
| EFNA5₂ (rs252802), d | 2.94 | (1.58-5.47) | 7.0 × 10⁻⁰⁴ | | | | |
| EFNA5₃ (rs6868410), r | 0.67 | (0.07-7.00) | 7.4 × 10⁻⁰¹ | | | | |
| EPHA4 (rs10498114), d | 0.38 | (0.21-0.68) | 1.2 × 10⁻⁰³ | | | | |
| EPHB1₁ (rs11917038), a | 0.93 | (0.49-1.76) | 8.2 × 10⁻⁰¹ | | | | |
| EPHB1₂ (rs1404577), r | 0.19 | (0.06-0.66) | 8.6 × 10⁻⁰³ | | | | |
| EPHB1₃ (rs6793828), r | 0.48 | (0.24-0.95) | 3.5 × 10⁻⁰² | | | | |
| EPHB1₄ (rs9865620), d | 0.24 | (0.13-0.45) | 6.8 × 10⁻⁰⁶ | | | | |
| EPHB2 (rs11800828), d | 1.89 | (1.07-3.35) | 3.0 × 10⁻⁰² | | | | |
| FYN₁ (rs11153311), a | 0.36 | (0.20-0.64) | 5.5 × 10⁻⁰⁴ | | | | |
| FYN₂ (rs809193), a | 2.31 | (1.48-3.62) | 2.4 × 10⁻⁰⁴ | | | | |
| GSK3B (rs3108749), a | 0.36 | (0.20-0.66) | 1.0 × 10⁻⁰³ | | | | |
| PAK7₁ (rs1033470), d | 2.58 | (1.51-4.43) | 5.7 × 10⁻⁰⁴ | | | | |
| PAK7₂ (rs219861), d | 0.53 | (0.30-0.93) | 2.6 × 10⁻⁰² | | | | |
| PLXNA2₁ (rs2782934), r | 0.19 | (0.07-0.51) | 1.0 × 10⁻⁰³ | | | | |
| PLXNA2₂ (rs6685511), d | 2.26 | (1.21-4.22) | 1.1 × 10⁻⁰² | | | | |
| PLXNC1₁ (rs10507033), r | 0.02 | (0.003-0.16) | 1.7 × 10⁻⁰⁴ | | | | |
| PLXNC1₂ (rs865569), r | 21.64 | (2.41-194.15) | 6.0 × 10⁻⁰³ | | | | |
| PPP3CA (rs2732504), d | 0.48 | (0.29-0.82) | 6.3 × 10⁻⁰³ | | | | |
| RAC2 (rs9610680), a | 1.95 | (1.19-3.21) | 8.2 × 10⁻⁰³ | | | | |
| RRAS2 (rs2970334), d | 0.32 | (0.17-0.60) | 4.2 × 10⁻⁰⁴ | | | | |
| SEMA5A₁ (rs1512508), d | 2.38 | (1.27-4.47) | 6.7 × 10⁻⁰³ | | | | |
| SEMA5A₂ (rs40701), r | 0.98 | (0.39-2.47) | 9.6 × 10⁻⁰¹ | | | | |
| SLIT3₁ (rs11746295), r | 0.14 | (0.03-0.58) | 6.7 × 10⁻⁰³ | | | | |
| SLIT3₂ (rs1979923), r | 0.20 | (0.08-0.53) | 1.2 × 10⁻⁰³ | | | | |
| SLIT3₃ (rs884787), d | 3.48 | (1.44-8.43) | 5.8 × 10⁻⁰³ | | | | |
| UNC5C (rs6820369), d | 2.22 | (1.03-4.76) | 4.1 × 10⁻⁰² | | | | |

*Subscript is used to identify individual SNPs in interactions.
**Results adjusted for age and gender.
^{†}a = log-additive, d = Mendelian dominant, r = Mendelian recessive.

The secondary whole-genome association dataset included 1,411 SNPs in the 26 genes from the model predicting age at onset of PD in the primary dataset. Of those SNPs, 142 (10.1%) were individually associated with survival free of PD (hazard function) using Cox proportional hazards models. Table 6 contains results for the final proportional hazards model produced by running SNPs through the multi-stage process to predict survival free of PD. This model used data from 263 PD patients (5 patients were missing data on one or more SNPs). In this case, the model had an overall p value of 6.30×10⁻³⁵. However, the model was not significant at predicting survival (age at study) of the matched sibling controls (p=0.14).

TABLE 6
Axon-guidance SNPs predicting survival free of Parkinson's disease, within a secondary
whole-genome association dataset.

| Gene* (rs ID), | Hazard Ratio | | | Hazard Ratio | |
|---|---|---|---|---|---|
| Final Coding Scheme^{†} | (95% CI) | P value | Interaction* | (95% CI) | P value |
| DCC₁ (rs1675225), a | 1.54 (1.12-2.13) | 8.70 × 10⁻⁰³ | NTNG1₂* ROBO1₁ | 0.25 (0.12-0.51) | 1.70 × 10⁻⁰⁴ |
| DCC₂ (rs12456286), r | 5.76 (2.16-15.37) | 4.70 × 10⁻⁰⁴ | | | |
| DPYSL2₁ (rs327216), a | 1.68 (1.16-2.44) | 6.00 × 10⁻⁰³ | | | |
| DPYSL2₂ (rs2585459), r | 0.60 (0.39-0.92) | 2.10 × 10⁻⁰² | | | |
| EPHB1₁ (rs868909), d | 0.58 (0.44-0.78) | 2.30 × 10⁻⁰⁴ | | | |
| EPHB1₂ (rs959562), d | 0.50 (0.33-0.74) | 5.50 × 10⁻⁰⁴ | | | |
| EPHB1₃ (rs6802089), a | 1.28 (1.04-1.59) | 2.20 × 10⁻⁰² | | | |
| EPHB1₄ (rs10512945), a | 1.59 (1.21-2.10) | 1.00 × 10⁻⁰³ | | | |
| NFATC2 (rs4811184), r | 4.59 (2.26-9.35) | 2.60 × 10⁻⁰⁵ | | | |
| NTNG1₁ (rs3813219), r | 0.26 (0.11-0.60) | 1.50 × 10⁻⁰³ | | | |
| NTNG1₂ (rs12133394), d | 0.75 (0.52-1.10) | 1.40 × 10⁻⁰¹ | | | |
| PLXNC1₁ (rs6538491), d | 0.55 (0.40-0.77) | 3.40 × 10⁻⁰⁴ | | | |
| PLXNC1₂ (rs10507031), a | 0.73 (0.60-0.90) | 2.30 × 10⁻⁰³ | | | |
| PLXNC1₃ (rs12579612), r | 2.83 (1.38-5.82) | 4.50 × 10⁻⁰³ | | | |
| PPP3CA (rs2732504), a | 0.75 (0.62-0.92) | 5.10 × 10⁻⁰³ | | | |
| ROBO1₁ (rs3773236), d | 2.42 (1.73-3.39) | 2.20 × 10⁻⁰⁷ | | | |
| ROBO1₂ (rs12486635), r | 1.72 (1.07-2.75) | 2.40 × 10⁻⁰² | | | |
| ROBO2₁ (rs2875436), r | 3.48 (1.19-10.20) | 2.30 × 10⁻⁰² | | | |
| ROBO2₂ (rs7614439), d | 1.45 (1.10-1.91) | 7.60 × 10⁻⁰³ | | | |
| ROBO2₃ (rs7640201), d | 4.65 (2.35-9.20) | 9.90 × 10⁻⁰⁶ | | | |
| ROCK2 (rs726843), r | 0.61 (0.45-0.84) | 2.10 × 10⁻⁰³ | | | |
| SEMA3E (rs2713178), a | 1.43 (1.17-1.76) | 5.80 × 10⁻⁰⁴ | | | |
| SEMA5A (rs1805958), r | 2.64 (1.31-5.32) | 6.70 × 10⁻⁰³ | | | |
| SLIT3₁ (rs11134568), r | 0.30 (0.13-0.70) | 5.70 × 10⁻⁰³ | | | |
| SLIT3₂ (rs11954621), d | 0.71 (0.53-0.95) | 2.30 × 10⁻⁰² | | | |
| SRAGP3₁ (rs370526), d | 0.76 (0.58-0.99) | 4.20 × 10⁻⁰² | | | |
| SRGAP3₂ (rs557377), r | 0.22 (0.12-0.39) | 2.70 × 10⁻⁰⁷ | | | |
| SRGAP3₃ (rs1043951), a | 1.72 (1.28-2.32) | 3.80 × 10⁻⁰⁴ | | | |
| SRGAP3₄ (rs3732977), r | 0.10 (0.03-0.32) | 1.10 × 10⁻⁰⁴ | | | |
| SRGAP3₅ (rs6793348), d | 0.63 (0.47-0.84) | 1.30 × 10⁻⁰³ | | | |
| SRGAP3₆ (rs17530358), r | 2.28 (1.41-3.69) | 8.30 × 10⁻⁰⁴ | | | |

*Subscript is used to identify individual SNPs in interactions.
^{†}a = log-additive, d = Mendelian dominant, r = Mendelian recessive.

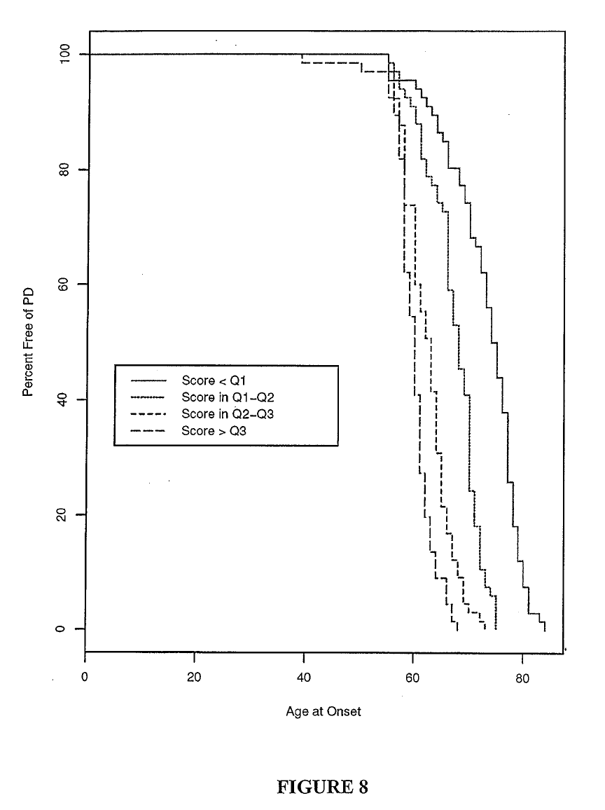

FIG. 8 uses a Kaplan-Meier plot to describe the results of the model. The groups were formed by calculating a risk score for each PD patient using the equation from the proportional hazards model, then categorizing the score at the 25th (Q1), 50th (Q2), and 75th (Q3) percentiles. The survival curves separated nicely right from the earliest ages of onset. By age 60, only 41% of PD patients in the predicted highest risk group were still free of PD, whereas 94% of PD patients in the predicted lowest risk group were free of PD. By age 70, none of the PD patients in the predicted highest risk group were still free of PD, whereas 68% of PD patients in the predicted lowest risk group were free of PD. The median ages at onset for each group, from lowest risk group to highest risk group, were 74, 68, 63, and 60, a difference in survival free of PD of 14 years from lowest to highest. The concordance for this model was 0.78. The hazard ratios (95% CIs) for the four groups, from lowest to highest risk, were 1 (reference), 4.02 (2.62-6.16), 10.69 (6.72-16.98), and 21.05 (12.85-34.48).

The secondary whole-genome association dataset included 1,605 SNPs in 28 of the 29 genes from the final model predicting age at onset of PD in our primary dataset. Of those SNPs, 157 (9.8%) were individually associated with age at onset of PD using linear regression models. Table 7 contains results for the final model produced by running SNPs through the multi-stage process to predict PD age at onset-squared. This model used data from 265 PD patients (3 patients were missing data on one or more SNPs). In this case, the model had an overall p value of 4.72×10⁻⁴⁰. However, the set of SNPs was not significant at predicting age at study-squared of the matched sibling controls (p=0.527).

TABLE 7
Axon-guidance SNPs predicting age at onset of Parkinson's disease, within a
secondary whole-genome association dataset.

| | Regression | | | | | | |
|---|---|---|---|---|---|---|---|
| Gene* (rs ID), | coefficient | | | | Regression coefficient | | |
| Final Coding Scheme^{†} | (standard error) | | P value | Interaction* | (standard error) | | P value |
| (Intercept) | 3795.40 | (147.38) | 4.3 × 10⁻⁷⁴ | NTNG1₂* NTNG1₁ | 1012.50 | (491.71) | 4.1 × 10⁻⁰² |
| ABLIM2 (rs1574393), d | −49.84 | (113.59) | 6.6 × 10⁻⁰¹ | PAK3* NTNG1₁ | −1400.09 | (635.70) | 2.9 × 10⁻⁰² |
| DCC₁ (rs11082952), d | 204.18 | (85.79) | 1.8 × 10⁻⁰² | DCC₁* DPYSL2 | 616.39 | (253.12) | 1.6 × 10⁻⁰² |
| DCC₂ (rs6508153), d | 494.94 | (75.56) | 3.0 × 10⁻¹⁰ | DPYSL2* ABLIM2 | 1081.60 | (269.80) | 8.1 × 10⁻⁰⁵ |
| DPYSL2 (rs2585459), r | 119.14 | (173.95) | 4.9 × 10⁻⁰¹ | EPHB1₃* SEMA5A | −525.25 | (232.19) | 2.5 × 10⁻⁰² |
| EFNA5 (rs252991), d | 271.73 | (78.45) | 6.2 × 10⁻⁰⁴ | EPHB1₁* SEMA3E₂ | −849.80 | (393.19) | 3.2 × 10⁻⁰² |
| EPHB1₁ (rs12488683), r | −375.90 | (140.38) | 7.9 × 10⁻⁰³ | EPHB1₁* SEMA3E₁ | 1264.51 | (467.74) | 7.4 × 10⁻⁰³ |
| EPHB1₂ (rs12489360), a | −233.83 | (58.97) | 9.4 × 10⁻⁰⁵ | SEMA3E₂* ABLIM2 | 546.98 | (240.42) | 2.4 × 10⁻⁰² |
| EPHB1₃ (rs2030737), a | 140.67 | (56.03) | 1.3 × 10⁻⁰² | SEMA3E₂* ROBO1₁ | 1190.80 | (370.50) | 1.5 × 10⁻⁰³ |
| EPHB1₄ (rs6439552), d | 514.37 | (139.71) | 2.8 × 10⁻⁰⁴ | SLIT2₁* EPHB1₄ | −682.99 | (220.23) | 2.2 × 10⁻⁰³ |
| NFATC2 (rs4811184), r | −671.00 | (190.98) | 5.2 × 10⁻⁰⁴ | SLIT3₂* ROBO1₁ | 1629.19 | (500.80) | 1.3 × 10⁻⁰³ |
| NTNG1₁ (rs11803905), r | 343.55 | (142.64) | 1.7 × 10⁻⁰² | | | | |
| NTNG1₂ (rs3813219), r | 450.90 | (312.93) | 1.5 × 10⁻⁰¹ | | | | |
| PAK3 (rs10521535), d | 752.30 | (229.63) | 1.2 × 10⁻⁰³ | | | | |
| PAK7 (rs7272361), d | −290.02 | (122.85) | 1.9 × 10⁻⁰² | | | | |
| ROBO1₁ (rs12486635), r | −430.73 | (135.39) | 1.7 × 10⁻⁰³ | | | | |
| ROBO1₂ (rs331146), d | −331.37 | (78.12) | 3.1 × 10⁻⁰⁵ | | | | |
| ROBO2₁ (rs1031377), r | 361.07 | (104.80) | 6.6 × 10⁻⁰⁴ | | | | |
| ROBO2₂ (rs6779560), r | 986.29 | (353.97) | 5.8 × 10⁻⁰³ | | | | |
| ROBO2₃ (rs7640201), d | −664.93 | (188.16) | 4.8 × 10⁻⁰⁴ | | | | |
| SEMA3E₁ (rs1639304), r | 281.84 | (188.58) | 1.4 × 10⁻⁰¹ | | | | |
| SEMA3E₂ (rs42002), r | −424.86 | (124.28) | 7.3 × 10⁻⁰⁴ | | | | |
| SEMA4D (rs9969727), r | 179.12 | (87.16) | 4.1 × 10⁻⁰² | | | | |
| SEMA5A (rs6873909), r | 16.38 | (306.90) | 9.6 × 10⁻⁰¹ | | | | |
| SLIT2₁ (rs10516357), d | −104.14 | (91.55) | 2.6 × 10⁻⁰¹ | | | | |
| SLIT2₂ (rs16869739), r | −1052.96 | (377.31) | 5.7 × 10⁻⁰³ | | | | |
| SLIT2₃ (rs644607), r | 1368.48 | (279.36) | 1.7 × 10⁻⁰⁶ | | | | |
| SLIT2₄ (rs9992591), r | 429.13 | (139.80) | 2.4 × 10⁻⁰³ | | | | |
| SLIT3₁ (rs1432906), d | 335.31 | (88.26) | 1.8 × 10⁻⁰⁴ | | | | |
| SLIT3₂ (rs2879135), r | −30.39 | (241.58) | 9.0 × 10⁻⁰¹ | | | | |
| SLIT3₃ (rs297838), r | 1030.35 | (317.25) | 1.3 × 10⁻⁰³ | | | | |
| SRGAP3₁ (rs3732977), r | 1122.79 | (316.61) | 4.6 × 10⁻⁰⁴ | | | | |
| SRGAP3₂ (rs557377), r | 833.81 | (152.83) | 1.1 × 10⁻⁰⁷ | | | | |

*Subscript is used to identify individual SNPs in interactions.
^{†}a = log-additive, d = Mendelian dominant, r = Mendelian recessive.

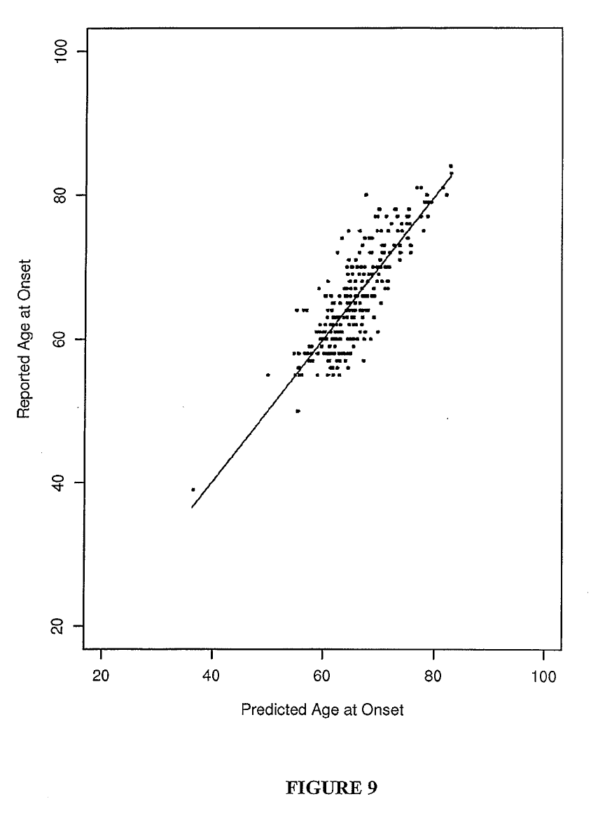

FIG. 9 shows a plot of predicted age at onset vs. reported age at onset to summarize the results of the model. The plot showed a nice elliptical pattern, reflecting the model R² of 0.714. The model explained about 71% of the variability in age at onset of PD.

Validation with a Gene Expression Profiling Dataset

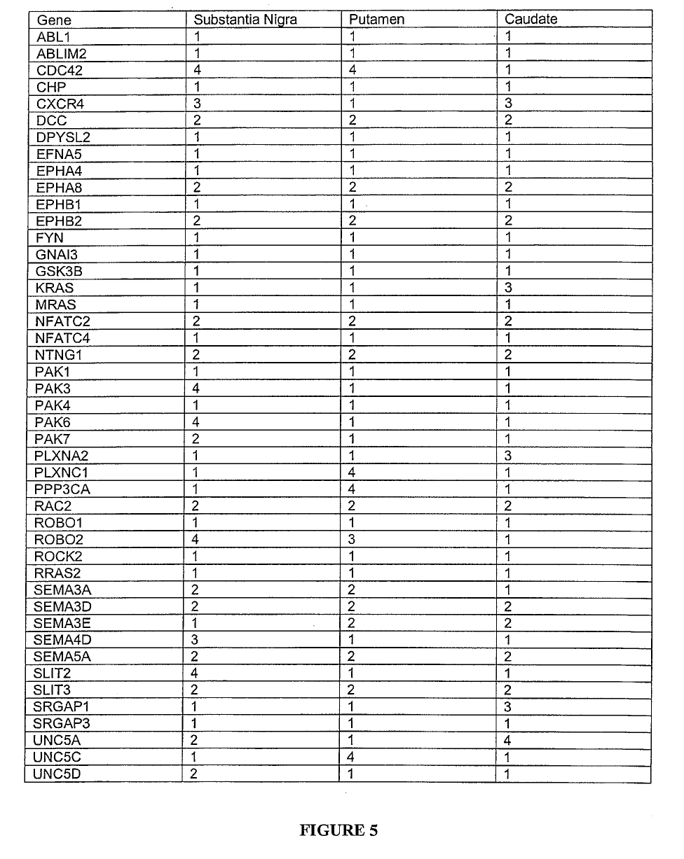

An available gene expression profiling dataset for PD that considered 21 different brain regions was mined. Details regarding the subjects, biological samples, and microarray experiments are described elsewhere (Papapetropoulos et al., Gene Expression, 13:205-215 (2006)). For this study, data analyses was limited to the substantia nigra and the striatum (putamen and caudate nuclei), since these are the brain regions contributing most significantly to the nigrostriatal dopamine deficiency that is characteristic of PD and since the three PD outcomes were defined according to the corresponding motor phenotype. There was a total of 45 genes represented by the SNPs listed for the three predictive genetic models (Tables 1-3), and the gene expression dataset had informative probe sets for 32 of those genes in the substantia nigra, 34 in the putamen, and 35 in the caudate. For the 45 axon guidance pathway genes represented by SNPs in the three predictive genetic models (as defined by the primary whole-genome association dataset), Table 8 provides detailed gene expression data for each of the three nigrostriatal regions considered, including probe set type, t-test p value, fold expression difference in cases and controls, and percent of cases and controls with expression present for the probe set. In each region, there were more differentially expressed genes observed than expected by chance: substantia nigra, 7 observed (22%) vs. 1.6 expected (5%); putamen, 5 observed (15%) vs. 1.7 expected (5%); and caudate, 5 observed (14%) vs. 1.8 expected (5%). Overall, 36 genes had data in at least one of the three regions, and 14 (39%) of those were differentially expressed in at least one region. FIG. 5 provides a detailed summary of the multiregional expression data for the 45 axon guidance pathway genes that were predictive of PD outcomes. No significant differences in the number of probe sets for differentially expressed versus normally expressed genes were observed, except for the putamen where two genes with eight fragments each were both differentially expressed. Removing those two genes (outliers) removed the significance (no differences in the median numbers of informative probe sets).

TABLE 8
Gene expression data for each of three nigrostriatal regions.

| | | SUBSTANTIA NIGRA | | | | PUTAMEN | | | | CAUDATE | | | |
|---|---|---|---|---|---|---|---|---|---|---|---|---|---|
| | | | | | % | | | | % | | | | % |
| Gene | | t-test | Fold | % Present | Present | t-test | Fold | % Present | Present | t-test | Fold | % Present | Present |
| Symbol | Probe Set ID | p-value | change | cases | controls | p-value | change | cases | controls | p-value | change | cases | controls |
| ABL1 | 202123_s_at | 0.024 | 1.22 | 100 | 100 | 0.520 | 1.08 | 100 | 100 | 0.150 | 1.15 | 100 | 100 |
| ABLIM2 | 1567622_at | 0.200 | 1.44 | 0 | 0 | 0.920 | 1.03 | 0 | 0 | 0.002 | −1.53 | 0 | 0 |
| ABLIM2 | 1567623_at | 0.300 | −1.37 | 9.09 | 0 | 0.820 | 1.04 | 0 | 0 | 0.460 | 1.29 | 0 | 0 |
| ABLIM2 | 215928_at | 0.930 | 1.02 | 27.27 | 25 | 0.870 | −1.04 | 87.5 | 92.31 | 0.630 | 1.14 | 60 | 100 |
| ABLIM2 | 228132_at | 0.310 | −1.14 | 100 | 93.75 | 0.440 | 1.33 | 87.5 | 100 | 0.600 | −1.12 | 100 | 100 |
| ABLIM2 | 236087_at | 0.120 | −1.42 | 63.64 | 31.25 | 1.000 | 1 | 75 | 69.23 | 0.370 | 1.15 | 90 | 100 |
| ABLIM2 | 242624_at | 0.053 | 1.78 | 0 | 6.25 | 0.620 | −1.12 | 0 | 0 | 0.370 | 1.28 | 0 | 7.14 |
| CDC42 | 1556931_at | 0.500 | 1.25 | 54.55 | 25 | 0.066 | −1.51 | 62.5 | 15.38 | 1.000 | −1 | 10 | 35.71 |
| CDC42 | 208727_s_at | 0.850 | −1.05 | 100 | 100 | 0.340 | −1.41 | 100 | 100 | 0.350 | 1.21 | 90 | 100 |
| CDC42 | 208728_s_at | 0.005 | −1.22 | 100 | 100 | 0.077 | −1.24 | 87.5 | 100 | 0.005 | −1.34 | 100 | 92.86 |
| CDC42 | 210232_at | 0.065 | −1.98 | 72.73 | 75 | 0.430 | −1.27 | 37.5 | 61.54 | 0.730 | −1.14 | 70 | 78.57 |
| CDC42 | 226400_at | 0.018 | −1.56 | 100 | 100 | 0.002 | −1.59 | 100 | 100 | 0.240 | −1.18 | 100 | 100 |
| CHP | 207993_s_at | 0.190 | −1.42 | 0 | 0 | 0.990 | 1.01 | 0 | 0 | 0.530 | 1.29 | 0 | 0 |
| CHP | 211369_at | 0.310 | 1.43 | 9.09 | 12.5 | 0.840 | 1.11 | 0 | 7.69 | 0.054 | −2.07 | 0 | 7.14 |
| CHP | 214665_s_at | 0.460 | −1.1 | 100 | 100 | 0.860 | −1.02 | 100 | 100 | 0.510 | −1.05 | 100 | 100 |
| CXCR4 | 209201_x_at | 0.004 | 1.89 | 0 | 43.75 | 0.200 | 1.59 | 12.5 | 7.69 | 0.250 | 1.41 | 20 | 42.86 |
| CXCR4 | 211919_s_at | 0.007 | 1.76 | 18.18 | 62.5 | 0.300 | 1.3 | 12.5 | 7.69 | 0.068 | 1.79 | 10 | 50 |
| CXCR4 | 217028_at | 0.003 | 2.21 | 100 | 100 | 0.280 | 1.48 | 100 | 92.31 | 0.030 | 2 | 100 | 100 |
| DCC | 206939_at | 0.029 | −2.3 | 0 | 0 | 0.094 | −1.84 | 0 | 0 | 0.940 | −1.04 | 0 | 0 |
| DCC | 242653_at | 0.880 | −1.06 | 0 | 6.25 | 0.090 | −1.66 | 12.5 | 0 | 0.110 | −1.6 | 10 | 7.14 |
| DPYSL2 | 1563482_at | 0.530 | −1.11 | 100 | 93.75 | 0.930 | −1.03 | 87.5 | 100 | 0.610 | 1.1 | 70 | 100 |
| DPYSL2 | 200762_at | 0.600 | −1.03 | 100 | 100 | 0.480 | −1.05 | 100 | 100 | 0.790 | −1.01 | 100 | 100 |
| EFNA5 | 1559360_at | 0.180 | 1.26 | 81.82 | 93.75 | 0.190 | 1.27 | 87.5 | 100 | 0.078 | 1.48 | 80 | 100 |
| EFNA5 | 207301_at | 0.460 | −1.31 | 0 | 0 | 0.079 | −1.9 | 0 | 0 | 0.700 | 1.11 | 0 | 0 |
| EPHA4 | 206114_at | 0.023 | 1.98 | 18.18 | 50 | 0.230 | 1.27 | 62.5 | 92.31 | 0.062 | 2.79 | 70 | 100 |
| EPHA4 | 227449_at | 0.210 | 1.4 | 90.91 | 100 | 0.860 | 1.03 | 100 | 100 | 0.840 | 1.03 | 100 | 100 |
| EPHA4 | 228948_at | 0.520 | 1.28 | 63.64 | 43.75 | 0.650 | −1.08 | 100 | 100 | 0.160 | 1.22 | 100 | 100 |
| EPHA4 | 229374_at | 0.630 | 1.1 | 45.45 | 37.5 | 0.220 | −1.29 | 50 | 76.92 | 0.710 | 1.07 | 80 | 100 |
| EPHA8 | 1554069_at | 0.220 | −1.43 | 0 | 0 | 0.050 | −1.7 | 0 | 0 | 0.770 | 1.09 | 0 | 0 |
| EPHA8 | 231796_at | 0.360 | 1.34 | 0 | 0 | 0.460 | −1.42 | 0 | 0 | 0.520 | −1.23 | 0 | 0 |
| EPHB1 | 210753_s_at | 0.810 | −1.1 | 0 | 6.25 | 0.050 | 1.25 | 37.5 | 76.92 | 0.630 | 1.06 | 40 | 92.86 |
| EPHB1 | 211898_s_at | 0.410 | 1.27 | 0 | 0 | 0.790 | 1.11 | 0 | 0 | 0.710 | 1.17 | 0 | 0 |
| EPHB1 | 217324_at | 0.980 | −1.01 | 27.27 | 6.25 | 0.590 | 1.2 | 12.5 | 38.46 | 0.580 | −1.16 | 10 | 0 |
| EPHB1 | 230425_at | 0.410 | 1.25 | 72.73 | 75 | 0.029 | 1.27 | 100 | 100 | 0.580 | 1.07 | 100 | 100 |
| EPHB2 | 209588_at | 0.320 | −1.22 | 0 | 0 | 0.440 | −1.32 | 0 | 0 | 0.310 | −1.4 | 0 | 0 |
| EPHB2 | 209589_s_at | 0.560 | −1.16 | 72.73 | 68.75 | 0.890 | −1.02 | 37.5 | 15.38 | 0.910 | −1.03 | 10 | 7.14 |
| EPHB2 | 210651_s_at | 0.850 | −1.03 | 45.45 | 56.25 | 0.630 | −1.18 | 25 | 0 | 0.690 | −1.13 | 40 | 21.43 |
| EPHB2 | 211165_x_at | 0.390 | 1.34 | 27.27 | 18.75 | 0.110 | 1.61 | 0 | 0 | 0.004 | 2.06 | 0 | 0 |
| EPHB2 | 233699_at | 0.100 | −1.91 | 0 | 0 | 0.450 | 1.3 | 0 | 0 | 0.230 | 1.49 | 0 | 0 |
| FYN | 1559101_at | 0.140 | 1.35 | 90.91 | 100 | 0.220 | 1.3 | 87.5 | 100 | 0.550 | 1.13 | 90 | 100 |
| FYN | 210105_s_at | 0.770 | 1.02 | 100 | 100 | 0.093 | −1.2 | 100 | 100 | 0.490 | −1.06 | 100 | 100 |
| FYN | 212486_s_at | 1.000 | −1 | 100 | 100 | 0.220 | −1.25 | 100 | 100 | 0.520 | −1.1 | 100 | 100 |
| FYN | 216033_s_at | 0.190 | 1.13 | 100 | 100 | 0.750 | −1.03 | 100 | 100 | 0.430 | 1.11 | 100 | 100 |
| FYN | 217697_at | 0.670 | 1.24 | 18.18 | 6.25 | 0.650 | −1.25 | 0 | 0 | 0.015 | −2.27 | 0 | 7.14 |
| FYN | 243006_at | 0.210 | 1.27 | 90.91 | 100 | 0.058 | −1.32 | 100 | 100 | 0.100 | −1.21 | 100 | 100 |
| GNAI3 | 201179_s_at | 0.450 | −1.06 | 54.55 | 75 | 0.420 | −1.16 | 37.5 | 46.15 | 0.650 | −1.06 | 70 | 85.71 |
| GNAI3 | 201180_s_at | 0.290 | 1.1 | 100 | 100 | 0.220 | −1.12 | 100 | 100 | 0.880 | 1.01 | 100 | 100 |
| GNAI3 | 201181_at | 0.500 | 1.1 | 90.91 | 68.75 | 0.550 | 1.1 | 62.5 | 69.23 | 0.880 | −1.02 | 70 | 85.71 |
| GSK3B | 209945_s_at | 0.054 | −1.34 | 100 | 100 | 0.650 | −1.09 | 100 | 100 | 0.100 | 1.52 | 100 | 100 |
| GSK3B | 226183_at | 0.180 | −1.28 | 100 | 100 | 0.120 | 1.19 | 100 | 100 | 0.850 | −1.02 | 100 | 100 |
| GSK3B | 226191_at | 0.120 | −1.21 | 100 | 100 | 0.890 | 1.02 | 100 | 100 | 0.620 | 1.04 | 100 | 100 |
| GSK3B | 242336_at | 0.490 | 1.26 | 36.36 | 50 | 0.260 | −1.39 | 37.5 | 38.46 | 0.600 | −1.14 | 50 | 28.57 |
| KRAS | 1559203_s_at | 0.320 | 1.42 | 18.18 | 18.75 | 0.140 | −1.51 | 25 | 7.69 | 0.830 | −1.11 | 20 | 14.29 |
| KRAS | 1559204_x_at | 0.190 | −1.45 | 45.45 | 12.5 | 0.640 | −1.21 | 12.5 | 7.69 | 0.086 | −1.51 | 20 | 21.43 |
| KRAS | 204009_s_at | 0.110 | −1.18 | 100 | 100 | 0.460 | −1.07 | 100 | 100 | 0.550 | 1.06 | 100 | 100 |
| KRAS | 204010_s_at | 0.170 | 1.18 | 100 | 100 | 0.860 | −1.04 | 100 | 92.31 | 0.270 | −1.17 | 90 | 100 |
| KRAS | 214352_s_at | 0.930 | −1.01 | 100 | 100 | 0.940 | 1.01 | 100 | 100 | 0.034 | 1.95 | 90 | 100 |
| MRAS | 206538_at | 0.041 | 1.37 | 72.73 | 68.75 | 0.310 | 1.3 | 75 | 84.62 | 0.810 | −1.07 | 70 | 92.86 |
| MRAS | 225185_at | 0.058 | 1.26 | 100 | 100 | 0.650 | 1.05 | 100 | 100 | 0.034 | 1.2 | 100 | 100 |
| NFATC2 | 226991_at | 0.091 | 1.67 | 9.09 | 18.75 | 0.069 | 1.56 | 37.5 | 23.08 | 0.180 | 1.43 | 10 | 64.29 |
| NFATC2 | 228442_at | 0.510 | 1.23 | 0 | 0 | 0.230 | 1.18 | 0 | 0 | 0.960 | 1.01 | 10 | 21.43 |
| NFATC2 | 233706_at | 0.850 | −1.09 | 0 | 0 | 0.410 | −1.53 | 0 | 0 | 0.110 | −1.75 | 0 | 0 |
| NFATC4 | 213345_at | 0.620 | 1.13 | 18.18 | 25 | 0.670 | −1.1 | 25 | 7.69 | 0.150 | −1.35 | 30 | 28.57 |
| NFATC4 | 236270_at | 0.280 | 1.28 | 63.64 | 81.25 | 0.490 | 1.1 | 87.5 | 84.62 | 0.940 | 1.01 | 100 | 100 |
| NTNG1 | 1561014_at | 0.045 | −2.02 | 0 | 6.25 | 0.710 | −1.17 | 12.5 | 0 | 0.910 | 1.05 | 0 | 0 |
| NTNG1 | 1561767_at | 0.760 | 1.1 | 9.09 | 12.5 | 0.640 | −1.25 | 0 | 0 | 0.390 | 1.51 | 0 | 7.14 |
| NTNG1 | 206713_at | 0.230 | −1.53 | 0 | 6.25 | 0.380 | −1.29 | 12.5 | 0 | 0.180 | −1.57 | 0 | 14.29 |
| NTNG1 | 236088_at | 0.240 | 1.43 | 63.64 | 43.75 | 0.680 | 1.1 | 25 | 7.69 | 0.980 | 1 | 20 | 50 |
| PAK1 | 209615_s_at | 0.920 | 1.04 | 36.36 | 12.5 | 0.860 | −1.09 | 25 | 15.38 | 0.470 | −1.23 | 20 | 21.43 |
| PAK1 | 226507_at | 0.890 | −1.03 | 100 | 100 | 0.570 | −1.11 | 100 | 100 | 0.480 | −1.11 | 100 | 100 |
| PAK3 | 214607_at | 0.045 | −1.43 | 100 | 100 | 0.440 | −1.11 | 100 | 100 | 0.120 | −1.29 | 100 | 100 |
| PAK4 | 203154_s_at | 0.910 | −1.01 | 0 | 6.25 | 0.260 | 1.2 | 0 | 7.69 | 0.520 | 1.15 | 0 | 14.29 |
| PAK4 | 33814_at | 0.210 | 1.14 | 90.91 | 100 | 0.600 | −1.07 | 100 | 100 | 0.980 | −1 | 100 | 100 |
| PAK6 | 1555310_b_at | 0.021 | −1.42 | 100 | 75 | 0.250 | −1.14 | 100 | 100 | 0.190 | −1.22 | 100 | 100 |
| PAK6 | 219461_at | 0.360 | −1.27 | 27.27 | 12.5 | 0.590 | −1.05 | 100 | 100 | 0.740 | −1.04 | 90 | 100 |
| PAK6 | 239470_at | 0.200 | 1.69 | 0 | 0 | 0.770 | 1.17 | 0 | 0 | 0.950 | −1.03 | 0 | 0 |
| PAK7 | 210721_s_at | 0.580 | 1.16 | 72.73 | 68.75 | 0.460 | 1.18 | 100 | 100 | 0.870 | −1.03 | 100 | 100 |
| PAK7 | 213990_s_at | 0.680 | 1.16 | 0 | 12.5 | 0.500 | 1.24 | 62.5 | 61.54 | 0.610 | −1.08 | 70 | 85.71 |
| PLXNA2 | 207290_at | 0.470 | −1.22 | 9.09 | 6.25 | 0.290 | 1.27 | 0 | 38.46 | 0.190 | 1.76 | 10 | 50 |
| PLXNA2 | 213030_s_at | 0.190 | 1.19 | 100 | 100 | 0.130 | 1.12 | 100 | 100 | 0.037 | 1.31 | 100 | 100 |
| PLXNA2 | 222159_at | 0.860 | 1.02 | 100 | 87.5 | 0.210 | −1.23 | 100 | 100 | 0.190 | −1.19 | 100 | 100 |
| PLXNA2 | 232474_at | 0.063 | 1.86 | 0 | 0 | 0.740 | 1.12 | 0 | 0 | 0.650 | 1.11 | 0 | 0 |
| PLXNA2 | 234636_at | 0.490 | 1.37 | 0 | 0 | 0.970 | −1.02 | 0 | 0 | 0.750 | −1.17 | 0 | 14.29 |
| PLXNA2 | 243890_at | 0.610 | −1.17 | 0 | 0 | 0.340 | 1.31 | 0 | 0 | 0.920 | 1.04 | 0 | 7.14 |
| PLXNC1 | 206470_at | 0.530 | −1.32 | 0 | 0 | 0.290 | −1.54 | 0 | 0 | 0.064 | 2.22 | 0 | 14.29 |
| PLXNC1 | 206471_s_at | 0.200 | 1.4 | 27.27 | 25 | 0.310 | −1.3 | 50 | 38.46 | 0.840 | 1.03 | 40 | 92.86 |
| PLXNC1 | 213241_at | 0.540 | 1.08 | 100 | 100 | 0.006 | −1.49 | 100 | 100 | 0.900 | 1.01 | 100 | 100 |
| PLXNC1 | 235328_at | 0.800 | −1.1 | 0 | 0 | 0.320 | −1.4 | 0 | 7.69 | 0.140 | 1.59 | 0 | 21.43 |
| PLXNC1 | 240638_at | 0.200 | 1.63 | 9.09 | 6.25 | 0.094 | −1.93 | 12.5 | 0 | 0.930 | −1.03 | 0 | 42.86 |
| PPP3CA | 1557637_at | 0.180 | 1.35 | 36.36 | 68.75 | 0.045 | −1.88 | 75 | 76.92 | 0.750 | 1.08 | 60 | 78.57 |
| PPP3CA | 1560552_b_at | 0.610 | 1.25 | 0 | 6.25 | 0.950 | 1.03 | 12.5 | 38.46 | 0.560 | 1.33 | 30 | 57.14 |
| PPP3CA | 1562467_at | 0.530 | −1.24 | 36.36 | 50 | 0.530 | 1.22 | 75 | 92.31 | 0.110 | 1.91 | 70 | 100 |
| PPP3CA | 1566201_at | 0.960 | −1.02 | 36.36 | 31.25 | 0.450 | 1.34 | 25 | 76.92 | 0.062 | −1.41 | 50 | 64.29 |
| PPP3CA | 1566202_at | 0.210 | 1.88 | 9.09 | 0 | 0.380 | 1.59 | 0 | 7.69 | 0.430 | −1.62 | 0 | 0 |
| PPP3CA | 202425_x_at | 0.065 | 1.24 | 100 | 100 | 0.003 | 1.6 | 100 | 100 | 0.021 | 1.59 | 100 | 100 |
| PPP3CA | 202429_s_at | 0.290 | 1.18 | 100 | 100 | 0.410 | 1.24 | 100 | 100 | 0.820 | −1.06 | 100 | 100 |
| PPP3CA | 202457_s_at | 0.210 | 1.18 | 100 | 100 | 0.074 | 1.21 | 100 | 100 | 0.960 | 1 | 100 | 100 |
| PPP3CA | 229606_at | 0.490 | −1.14 | 100 | 100 | 0.720 | −1.08 | 100 | 100 | 0.980 | −1.01 | 100 | 100 |
| PPP3CA | 236545_at | 0.063 | 1.27 | 100 | 100 | 0.420 | 1.16 | 100 | 100 | 0.510 | 1.12 | 100 | 100 |
| RAC2 | 207419_s_at | 0.400 | −1.49 | 0 | 0 | 0.120 | −1.91 | 0 | 0 | 0.100 | −2.02 | 0 | 0 |
| RAC2 | 213603_s_at | 0.290 | 1.48 | 27.27 | 37.5 | 0.690 | −1.16 | 25 | 7.69 | 0.210 | 1.23 | 0 | 14.29 |
| ROBO1 | 1558797_at | 0.980 | −1 | 54.55 | 50 | 0.930 | 1.04 | 50 | 53.85 | 1.000 | −1 | 60 | 42.86 |
| ROBO1 | 213194_at | 0.460 | 1.08 | 100 | 100 | 0.670 | 1.04 | 100 | 100 | 0.650 | 1.03 | 100 | 100 |
| ROBO1 | 240558_at | 0.830 | −1.09 | 9.09 | 6.25 | 0.087 | −2.3 | 0 | 0 | 0.750 | −1.18 | 0 | 14.29 |
| ROBO2 | 226709_at | 0.015 | −2.26 | 81.82 | 25 | 0.003 | 1.49 | 37.5 | 92.31 | 0.110 | 1.47 | 70 | 100 |
| ROBO2 | 226766_at | 0.002 | −2.9 | 100 | 75 | 0.950 | −1.01 | 100 | 100 | 0.230 | −1.22 | 100 | 100 |
| ROCK2 | 202762_at | 0.016 | 1.23 | 100 | 100 | 0.044 | 1.29 | 100 | 100 | 0.880 | 1.02 | 100 | 100 |
| ROCK2 | 211504_x_at | 0.630 | −1.06 | 100 | 100 | 0.530 | −1.09 | 100 | 100 | 0.600 | 1.09 | 100 | 100 |
| RRAS2 | 208456_s_at | 0.760 | −1.1 | 54.55 | 62.5 | 0.480 | 1.12 | 37.5 | 76.92 | 0.790 | −1.04 | 80 | 71.43 |
| RRAS2 | 212589_at | 0.180 | −1.2 | 100 | 100 | 0.068 | −1.19 | 100 | 100 | 0.640 | 1.06 | 100 | 100 |
| RRAS2 | 212590_at | 0.330 | −1.16 | 81.82 | 93.75 | 0.056 | −1.24 | 100 | 92.31 | 0.510 | −1.08 | 90 | 100 |
| SEMA3A | 206805_at | 0.470 | −1.27 | 9.09 | 6.25 | 0.510 | −1.15 | 62.5 | 46.15 | 0.260 | 1.16 | 60 | 100 |
| SEMA3A | 244163_at | 0.550 | −1.29 | 9.09 | 0 | 0.420 | −1.6 | 50 | 15.38 | 0.460 | −1.31 | 20 | 35.71 |
| SEMA3A | 244849_at | 0.700 | −1.17 | 0 | 0 | 0.002 | −4.9 | 0 | 0 | 0.078 | −1.9 | 0 | 0 |
| SEMA3D | 215324_at | 0.970 | −1.02 | 0 | 0 | 0.190 | 1.81 | 0 | 7.69 | 0.530 | −1.27 | 0 | 0 |
| SEMA3D | 215643_at | 0.150 | 1.47 | 36.36 | 56.25 | 0.210 | −1.46 | 50 | 53.85 | 0.027 | −1.42 | 50 | 57.14 |
| SEMA3D | 243373_at | 0.260 | 1.58 | 0 | 0 | 0.770 | −1.19 | 0 | 0 | 0.550 | 1.3 | 0 | 0 |
| SEMA3E | 206941_x_at | 0.540 | −1.09 | 81.82 | 93.75 | 0.340 | 1.85 | 37.5 | 69.23 | 0.780 | −1.12 | 10 | 28.57 |
| SEMA4D | 1562250_at | 0.370 | 1.22 | 72.73 | 50 | 0.270 | 1.79 | 37.5 | 30.77 | 0.410 | −1.38 | 20 | 35.71 |
| SEMA4D | 203528_at | 0.011 | 1.4 | 100 | 100 | 0.280 | 1.2 | 100 | 100 | 0.980 | −1 | 100 | 100 |
| SEMA5A | 1560889_b_at | 0.750 | 1.16 | 9.09 | 0 | 0.580 | 1.31 | 0 | 7.69 | 0.660 | 1.18 | 0 | 0 |
| SEMA5A | 1563331_at | 0.430 | 1.34 | 0 | 0 | 0.920 | −1.04 | 0 | 0 | 0.430 | 1.39 | 0 | 0 |
| SEMA5A | 1567705_at | 0.770 | 1.11 | 9.09 | 0 | 0.870 | −1.09 | 0 | 0 | 0.980 | −1.01 | 0 | 0 |
| SEMA5A | 1567706_at | 0.340 | −1.48 | 0 | 6.25 | 0.460 | 1.57 | 0 | 0 | 0.059 | −2.61 | 0 | 0 |
| SEMA5A | 205405_at | 0.530 | −1.1 | 54.55 | 37.5 | 0.640 | −1.11 | 0 | 7.69 | 0.530 | 1.24 | 10 | 7.14 |
| SLIT2 | 209897_s_at | 0.510 | −1.09 | 100 | 93.75 | 0.810 | −1.04 | 100 | 100 | 0.950 | 1.01 | 100 | 100 |
| SLIT2 | 228850_s_at | 0.096 | −1.25 | 90.91 | 93.75 | 0.330 | −1.13 | 100 | 92.31 | 0.450 | −1.11 | 100 | 100 |
| SLIT2 | 230130_at | 0.020 | −1.69 | 100 | 93.75 | 0.330 | −1.25 | 100 | 100 | 0.400 | −1.17 | 100 | 100 |
| SLIT3 | 1561574_at | 0.850 | 1.06 | 0 | 0 | 0.950 | −1.03 | 0 | 7.69 | 0.970 | −1.01 | 0 | 7.14 |
| SLIT3 | 203812_at | 0.500 | −1.1 | 27.27 | 12.5 | 0.830 | −1.04 | 50 | 61.54 | 0.150 | −1.2 | 40 | 64.29 |
| SLIT3 | 203813_s_at | 0.990 | −1 | 9.09 | 12.5 | 0.170 | 1.43 | 12.5 | 15.38 | 0.650 | 1.1 | 0 | 0 |
| SLIT3 | 216216_at | 0.035 | 2.07 | 9.09 | 6.25 | 0.330 | 1.35 | 0 | 7.69 | 0.120 | −1.69 | 0 | 0 |
| SRGAP1 | 1554473_at | 0.280 | 1.26 | 81.82 | 87.5 | 0.610 | −1.13 | 87.5 | 100 | 0.160 | 1.39 | 90 | 100 |
| SRGAP1 | 1569269_s_at | 0.370 | −1.15 | 0 | 0 | 0.300 | −1.23 | 12.5 | 0 | 0.420 | 1.19 | 20 | 0 |
| SRGAP1 | 233888_s_at | 0.910 | 1.01 | 100 | 100 | 0.940 | 1.01 | 100 | 100 | 0.032 | 1.42 | 90 | 100 |
| SRGAP3 | 1559333_at | 0.730 | 1.13 | 0 | 0 | 0.200 | 1.6 | 0 | 0 | 0.940 | −1.04 | 0 | 0 |
| SRGAP3 | 1563595_at | 0.013 | 1.8 | 9.09 | 25 | 0.160 | −1.79 | 12.5 | 7.69 | 0.970 | 1.01 | 0 | 7.14 |
| SRGAP3 | 209794_at | 0.820 | −1.04 | 100 | 100 | 0.130 | −1.18 | 100 | 100 | 0.630 | 1.04 | 100 | 100 |
| SRGAP3 | 215550_at | 0.210 | 1.3 | 36.36 | 25 | 0.510 | 1.36 | 0 | 0 | 0.004 | 3.85 | 0 | 21.43 |
| SRGAP3 | 232869_at | 0.340 | 1.39 | 9.09 | 0 | 0.005 | −2.79 | 0 | 0 | 0.620 | 1.17 | 0 | 0 |
| SRGAP3 | 232954_at | 0.270 | 1.19 | 0 | 37.5 | 0.990 | −1 | 25 | 0 | 0.860 | −1.03 | 20 | 28.57 |
| SRGAP3 | 237080_at | 0.200 | 1.43 | 0 | 12.5 | 0.820 | 1.09 | 0 | 0 | 0.740 | 1.11 | 0 | 0 |
| UNC5A | 236448_at | 0.190 | −1.51 | 9.09 | 12.5 | 0.670 | −1.07 | 87.5 | 76.92 | 0.037 | −1.45 | 90 | 71.43 |
| UNC5A | 243833_at | 0.990 | 1 | 0 | 0 | 0.270 | −1.55 | 0 | 0 | 0.240 | −1.48 | 0 | 0 |
| UNC5C | 1555341_at | 0.640 | 1.22 | 0 | 0 | 0.036 | −3.33 | 0 | 0 | 0.460 | −1.46 | 0 | 0 |
| UNC5C | 1555342_b_at | 0.880 | 1.07 | 9.09 | 0 | 0.840 | 1.09 | 0 | 0 | 0.790 | 1.08 | 0 | 0 |
| UNC5C | 206189_at | 0.690 | 1.05 | 100 | 93.75 | 0.460 | −1.15 | 87.5 | 76.92 | 0.560 | 1.1 | 20 | 85.71 |
| UNC5C | 240958_at | 0.230 | 1.25 | 18.18 | 25 | 0.890 | 1.06 | 12.5 | 15.38 | 0.028 | −1.64 | 20 | 28.57 |
| UNC5C | 241873_at | 0.060 | −1.37 | 100 | 100 | 0.011 | −1.56 | 100 | 92.31 | 0.080 | −1.34 | 100 | 100 |
| UNC5D | 231325_at | 0.730 | 1.09 | 63.64 | 43.75 | 0.890 | −1.02 | 87.5 | 76.92 | 0.340 | −1.23 | 70 | 78.57 |

Very similar results were found from the sensitivity analyses treating accurate-type and cross-reacting type fragments equally. Again, in each region, there were more differentially expressed genes than expected by chance: substantia nigra, 22 observed (24%) vs. 4.7 expected (5%); putamen 11 observed (12%) vs. 4.7 expected (5%); and caudate, 16 observed (17%) vs. 4.7 expected (5%). Restricting to the 45 genes represented in the three predictive genetic models, the results were: substantia nigra, 7 observed (22%) vs. 1.6 expected (5%); putamen, 5 observed (15%) vs. 1.7 expected (5%); and caudate, 7 observed (20%) vs. 1.8 expected (5%). Overall, of the 36 genes with data in at least one of the three regions, 14 (39%) were still differentially expressed in at least one region.

The raw data from the original gene expression profiling study were re-normalized and re-analyzed using a different standard software package, in order to obtain results for all probe sets in all 45 genes and in all three regions. Differential expression of axon guidance pathway genes in PD was again observed, although the findings were more modest. Thirteen genes were differentially expressed in at least one region (8 in the substantia nigra, 1 in the putamen, 4 in the caudate).

For all analyses of differential gene expression, it was possible to code the differential expression unambiguously as increased or reduced. For genes with multiple informative and differentially expressed probe sets, the direction of effect was always the same.

The results provided herein provide compelling evidence that genetic variability in the axon guidance pathway predisposes to PD. The scope and magnitude of the effect was sizeable. For example, polymorphism in the axon guidance pathway accounted for nearly 70% of the age at onset variance of PD, and persons with high versus low model scores differed in age at onset by more than 20 years. The observed P values are likely amongst the smallest ever observed for a genetic analysis of PD or any complex disease, and withstand traditional adjustments for multiple comparisons.

The SNPs that were independent predictors of susceptibility or age at onset of PD may be only indirect markers of the functional variants within the genes. Fine mapping of these gene loci in diverse populations and experimental studies in transfected cell lines or in transgenic animals may help to elucidate the pathogenic mechanism of the axon guidance pathway variability that was observed. It is postulated that polymorphism in the axon guidance pathway genes results in aberrant trajectory of the ascending dopaminergic pathway during embryonic brain development and thus renders persons congenitally deficient in nigrostriatal dopamine. In other words, some people are wired differently from birth and are thus at greater risk to develop PD and at an earlier age.

In summary, multiple SNPs in axon guidance pathway genes were strong predictors of susceptibility and age at onset of PD. These findings might also suggest a new focus on environmental exposures that occur during intrauterine life. This study demonstrates that the combination of bioinformatic characterization of pathways with analysis of available whole-genome datasets can make important contributions to the understanding of complex diseases. The results provide evidence that complex diseases can be due to the joint effects of many genes which, taken singly, would show only small effects (“additive effects model”, “epistasis”) (Fisher, Trans. R. Soc. Edin. 52:399 (1918); Burton et al., Lancet 366:941 (2005); W. Batteson, Mendel's Principles of Heredity. (Cambridge Univ. Press, Cambridge, 1993); and Cordell and Clayton, Lancet 366, 1121 (2005)). They also provide an example of how a complex disease that appears to be largely sporadic and non-genetic can in fact have a strong genetic component (McDonnell et al., Ann. Neurol. 59, 788 (2006); Tanner et al., JAMA 281:341 (1999); and Rocca et al., Ann. Neurol. 56:495 (2004)).

### Example 2

### Attempts to Cripple the Axon Guidance Models by Removing SNPs

An attempted was made to cripple the models by removing SNPs in the reverse order from which they were selected, which should generally remove the single most important SNP first. The results for the first 10 SNPs removed from each model were as set forth in Table 9, 10, and 11.

TABLE 9
Susceptibility.

| Number of Removed SNPs | SNP | Gene | Model P value |
|---|---|---|---|
| 0 | | | 4.6E−38 |
| 1 | rs2044041 | PPP3CA | 2.4E−33 |
| 2 | rs4678260 | MRAS | 1.8E−29 |
| 3 | rs6656034 | PLXNA2 | 1.0E−27 |
| 4 | rs739043 | RAC2 | 3.7E−25 |
| 5 | rs17641276 | PAK4 | 2.6E−22 |
| 6 | rs2072952 | PAK7 | 9.2E−21 |
| 7 | rs10917325 | EPHB2 | 2.4E−19 |
| 8 | rs6492998 | CHP | 9.5E−19 |
| 9 | rs12740705 | CDC42 | 4.4E−17 |
| 10 | rs11185076 | NTNG1 | 2.1E−16 |

TABLE 10
Survival free of PD.

| Number of Removed SNPs | SNP | Gene | Model P value |
|---|---|---|---|
| 0 | | | 5.4E−48 |
| 1 | rs3822787 | SEMA5A | 2.2E−46 |
| 2 | ss46555247 | NFATC2 | 2.0E−44 |
| 3 | rs6769328 | ROBO1 | 7.9E−42 |
| 4 | rs17015294 | ROBO2 | 2.2E−36 |
| 5 | rs6762693 | SRGAP3 | 4.3E−34 |
| 6 | rs215285 | SEMA3E | 6.5E−32 |
| 7 | rs10008860 | ABLIM2 | 9.3E−32 |
| 8 | rs13256961 | UNC5D | 7.8E−32 |
| 9 | rs36183 | EPHB1 | 3.0E−30 |
| 10 | rs1432899 | SLIT3 | 1.7E−27 |

TABLE 11
Age at PD onset

| Number of Removed SNPs | SNP | Gene | Model P value |
|---|---|---|---|
| 0 | | | 1.7E−51 |
| 1 | ss46540509 | EPHB1 | 1.1E−45 |
| 2 | rs2295298 | NFATC4 | 1.1E−40 |
| 3 | rs4579786 | NTNG1 | 3.2E−40 |
| 4 | rs152562 | EFNA5 | 2.1E−39 |
| 5 | rs10784373 | SRGAP1 | 7.6E−39 |
| 6 | ss46555247 | NFATC2 | 2.2E−35 |
| 7 | rs1432899 | SLIT3 | 1.5E−32 |
| 8 | rs17015294 | ROBO2 | 8.2E−29 |
| 9 | rs6769328 | ROBO1 | 2.1E−28 |
| 10 | rs3822787 | SEMA5A | 1.8E−27 |

While the biggest reductions in the overall model p-values came from removing the first four SNPs in each table, even removing ten SNPs (plus their interactions) from each model left highly significant p-values. These results demonstrate that the models are remarkably robust.

### Example 3

### Axon Guidance Pathway Genes and ALS

This study employed a genomic pathway approach to determine whether polymorphism in the axon guidance pathway predisposed to ALS. Specifically, bioinformatic methods were used to mine an available whole-genome association dataset for SNPs that were within brain-expressed, axon guidance pathway genes. Then, statistical methods were used to construct models of axon guidance pathway SNPs that predicted three outcomes: ALS susceptibility, survival free of ALS, and age at onset of ALS. The primary whole-genome association study dataset employed by this study included 275 ALS cases and 269 unrelated controls. The median age at onset of ALS among the cases was 54 years (range 26-87). Details regarding the SNP markers genotyped, including call rates, Hardy-Weinberg equilibrium estimations, and re-genotyping concordance rates are described are described elsewhere (Schymick et al., Lancet Neurol., 6(4):322-8 (2007)). The bioinformatic methods identified 128 brain-expressed axon guidance pathway genes, and the SNP dataset included 4,133 SNPs within 124 of those genes.

All statistical tests were two-tailed, and considered significant at the conventional alpha level of 0.05. All statistical analyses were performed in SAS v. 9.1 (SAS Institute Inc., Cary, N.C.) or S-Plus v. 7 (Insightful Corp., Seattle, Wash.). Three outcomes of interest were considered: 1) ALS susceptibility, 2) survival free of ALS, and 3) age at onset of ALS. One goal was to identify joint action models of SNPs from the axon guidance pathway that predicted each of the three outcomes.

For the first outcome, unconditional logistic regressions were used to examine associations of the SNPs with ALS susceptibility while adjusting for age and gender. For each SNP, odds ratios (ORs), 95% confidence intervals (CIs), and p values were calculated. Goodness-of-fit was assessed through measuring concordance and visually through histograms of predicted probabilities. Overall odds ratios were estimated by categorizing the predicted probability of ALS from the model into four groups (<0.25, 0.25-0.50, 0.50-0.75, and >0.75), and then calculating the odds ratios for each group relative to the <0.25 group. A likelihood ratio test was used to assess the significance of the overall model. A 95% bias-corrected bootstrap CI was calculated for the associated p value using 10,000 re-samples.

For the second outcome, Cox proportional hazards models were used to test for associations of the SNPs with survival free of ALS. For each SNP, hazard ratios (HRs), 95% confidence intervals, and p values were calculated. Concordance was again calculated for the proportional hazards models, and Kaplan-Meier plots of categorized scores predicting risk of ALS were generated to provide visual gauges for goodness-of-fit. Hazard ratios were calculated for risk groups categorized at the quartiles, using the lowest risk group as reference. A likelihood ratio test was used to assess the significance of the overall model. A 95% bias-corrected bootstrap CI was calculated for the associated p value using 10,000 re-samples.

For the third outcome, the reported age at onset of ALS was predicted using multiple regression models. Goodness-of-fit was described through the model R² values and plots of the predicted vs. observed ages at onset. An F test was used to assess the significance of the overall model. A 95% bias-corrected bootstrap CI was calculated for the associated p value and the R² using 10,000 re-samples. Assumptions were tested throughout. Tests of linkage disequilibrium in unaffected siblings for the SNPs in the final models for the three outcomes were performed using LDSELECT v. 1.0 (copyright 2004 by Deborah A. Nickerson, Mark Rieder, Chris Carlson, Qian Yi, University of Washington) with a threshold R² of 0.80.

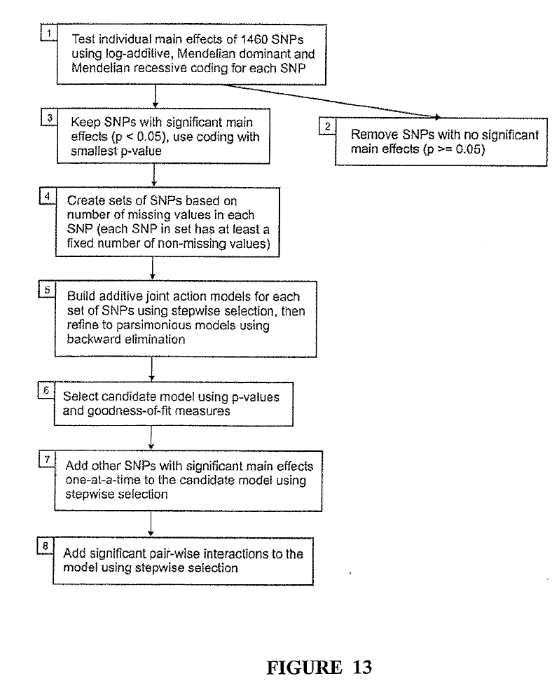

FIG. 13 summarizes the scheme used to develop models for each outcome. Since the modes of expression of the alleles in the SNPs of interest were not known, we first looked at each SNP using three coding schemes: log-additive, Mendelian dominant, and Mendelian recessive (Step 1). We simplified subsequent analyses by removing from further consideration those SNPs with no significant main effects in any coding scheme (Step 2). Removing those SNPs was a conservative approach, potentially biasing the tests towards the null hypotheses, since the SNPs were prevented from possibly entering the joint action models after adjustment for other variables. We generally coded the remaining SNPs using the schemes which produced the smallest p values, since these provided best estimates of the modes of expression in the data (Step 3). For each outcome, we then created multiple sets of SNPs, where each set contained only SNPs with at least a certain number of non-missing values (Step 4). This was done to address issues due to missing values.

While most SNPs had fairly complete data, others had missing values from substantial (up to 28%) numbers of subjects. We therefore chose an approach where we constructed candidate models using sets of SNPs with fairly complete data (effective sample sizes close to the maximum) to explain as much of the outcomes as possible, then checked to see if adding other SNPs on top of the candidate models would contribute significantly. We constructed the candidate models for each set using standard automated procedures (Step 5), and selected a final candidate model for each outcome based on significance and goodness-of-fit (Step 6). We then added other SNPs, which were significant given the candidate models (Step 7), and significant pair-wise interactions (Step 8).

To compare the significance of our axon guidance pathway SNP models to the significance of randomly selected genomic SNP models, we constructed 5,000 models for each outcome by randomly selecting the appropriate number of SNPs from the entire available data set. We then plotted the distributions of the test statistics from those models and the values of the test statistics from the final models.

### ALS Susceptibility

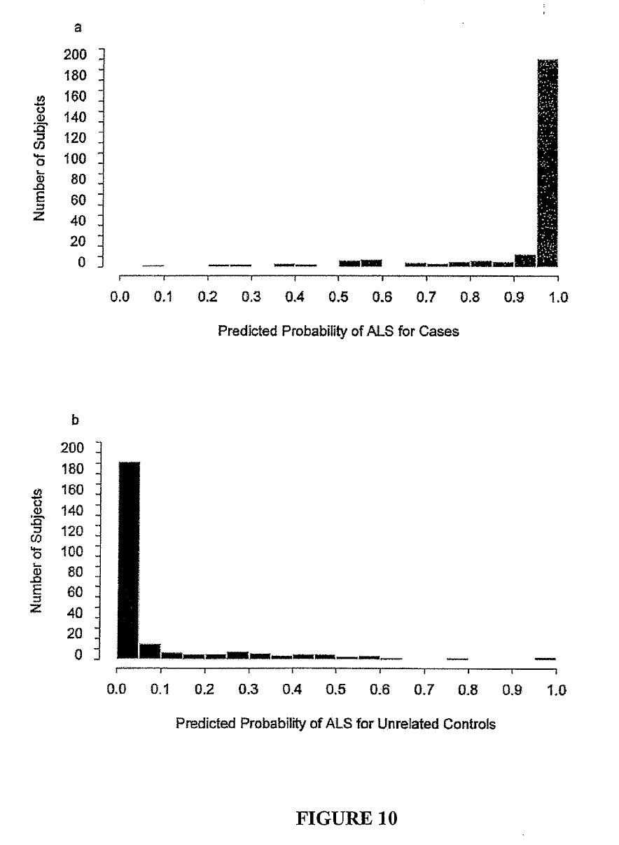

Of the 4,133 SNPs within brain-expressed genes of the axon-guidance pathway, 442 SNPs (10.7%) were individually associated with susceptibility to ALS, as detailed in Table 12. Table 13 contains results for the final model produced by running SNPs through the multi-stage process to predict ALS susceptibility. This model used data from 542 unmatched ALS patients and unrelated controls (2 subjects were missing data on one or more SNPs). The odds ratios (95% CIs) for the groups defined by predicted ALS probability of <0.25, 0.25-0.50, 0.50-0.75, and >0.75 were as follows: 1 (reference), 17.60 (5.70-54.36), 112.00 (35.45-353.83), and 1739.73 (523.53-5781.32) respectively. Since the interest was in the significance of the pathway, rather than individual SNPs, the p value for the overall model was of primary importance. In this case, the model had an overall p value of 2.92×10⁻⁶⁰ (95% CI 8.34×10⁻⁵²-1.16×10⁻⁶⁸). This model significantly predicted whether or not an individual was a case or an unrelated control. The predicted probabilities of ALS were very high (towards 1) for most of the cases, and very low (towards 0) for most of the unrelated controls (FIG. 10). Indeed, 78% of the cases had predicted probabilities above 0.9, and 77% of unrelated controls had predicted probabilities below 0.1. However, as shown by FIG. 10, the model did not completely distinguish the two groups; some cases had low predicted probabilities, and some controls had high predicted probabilities. The concordance for the model was about 0.99, indicating excellent agreement between predicted and observed case/control status.

TABLE 12
Axon-guidance SNPs.

| ID | Gene | rsID | Chromosome | Contig Position | allele1 | allele2 |
|---|---|---|---|---|---|---|
| 1 | EPHA2 | rs17416878 | 1 | 16319567 | A | G |
| 2 | EPHA2 | rs11260711 | 1 | 16322333 | C | T |
| 3 | EPHA2 | rs11260721 | 1 | 16327639 | T | C |
| 4 | EPHA2 | rs10907223 | 1 | 16332332 | G | A |
| 5 | EPHA2 | rs11260745 | 1 | 16338590 | T | C |
| 6 | EPHA2 | rs3768293 | 1 | 16340511 | A | C |
| 7 | EPHA2 | rs11260822 | 1 | 16358398 | T | C |
| 8 | CDC42 | rs2501275 | 1 | 22247652 | A | G |
| 9 | CDC42 | rs2473323 | 1 | 22261914 | A | C |
| 10 | CDC42 | rs2473322 | 1 | 22262659 | A | C |
| 11 | CDC42 | rs2056975 | 1 | 22281114 | C | T |
| 12 | CDC42 | rs12740705 | 1 | 22293700 | C | T |
| 13 | CDC42 | rs4076750 | 1 | 22296130 | T | C |
| 14 | EPHA8 | rs209736 | 1 | 22763996 | T | C |
| 15 | EPHA8 | rs11580191 | 1 | 22775110 | C | T |
| 16 | EPHA8 | rs649754 | 1 | 22778834 | T | G |
| 17 | EPHA8 | rs671485 | 1 | 22789451 | A | G |
| 18 | EPHA8 | rs209696 | 1 | 22792491 | C | T |
| 19 | EPHA8 | rs209693 | 1 | 22794613 | T | G |
| 20 | EPHA8 | rs209690 | 1 | 22795387 | G | A |
| 21 | EPHA8 | rs663002 | 1 | 22798200 | C | A |
| 22 | EPHA8 | rs12132543 | 1 | 22805640 | G | A |
| 23 | EPHB2 | rs294218 | 1 | 22905844 | A | C |
| 24 | EPHB2 | rs734445 | 1 | 22917415 | C | T |
| 25 | EPHB2 | rs2869513 | 1 | 22918530 | T | C |
| 26 | EPHB2 | rs882025 | 1 | 22922939 | G | A |
| 27 | EPHB2 | rs12732926 | 1 | 22929777 | G | A |
| 28 | EPHB2 | rs6687487 | 1 | 22934138 | G | A |
| 29 | EPHB2 | rs10917284 | 1 | 22935495 | G | A |
| 30 | EPHB2 | rs4612601 | 1 | 22939631 | A | G |
| 31 | EPHB2 | rs12043268 | 1 | 22940797 | C | T |
| 32 | EPHB2 | rs6673115 | 1 | 22942236 | C | T |
| 33 | EPHB2 | rs876685 | 1 | 22945007 | A | G |
| 34 | EPHB2 | rs4233292 | 1 | 22952938 | A | G |
| 35 | EPHB2 | rs893964 | 1 | 22958683 | G | A |
| 36 | EPHB2 | rs1561625 | 1 | 22959470 | G | A |
| 37 | EPHB2 | rs2582022 | 1 | 22959831 | A | G |
| 38 | EPHB2 | rs4654814 | 1 | 22967008 | C | T |
| 39 | EPHB2 | rs4655107 | 1 | 22967041 | G | A |
| 40 | EPHB2 | rs7516175 | 1 | 22969405 | A | C |
| 41 | EPHB2 | rs12410046 | 1 | 22971569 | G | A |
| 42 | EPHB2 | rs1561624 | 1 | 22974275 | C | A |
| 43 | EPHB2 | rs4655108 | 1 | 22976971 | G | A |
| 44 | EPHB2 | rs2817909 | 1 | 22978800 | A | G |
| 45 | EPHB2 | rs750012 | 1 | 22983092 | C | T |
| 46 | EPHB2 | rs2817896 | 1 | 22988636 | A | G |
| 47 | EPHB2 | rs893961 | 1 | 22990760 | A | G |
| 48 | EPHB2 | rs10917300 | 1 | 22990785 | T | C |
| 49 | EPHB2 | rs1465889 | 1 | 22992803 | G | A |
| 50 | EPHB2 | rs1866790 | 1 | 22996176 | G | A |
| 51 | EPHB2 | rs4506436 | 1 | 23000463 | C | T |
| 52 | EPHB2 | rs2817876 | 1 | 23009209 | C | — |
| 53 | EPHB2 | rs2119320 | 1 | 23011495 | G | A |
| 54 | EPHB2 | rs2869511 | 1 | 23013287 | A | G |
| 55 | EPHB2 | rs7536195 | 1 | 23015083 | C | T |
| 56 | EPHB2 | rs751022 | 1 | 23016234 | C | T |
| 57 | EPHB2 | rs893966 | 1 | 23016593 | G | A |
| 58 | EPHB2 | rs6667416 | 1 | 23017826 | G | A |
| 59 | EPHB2 | rs12090415 | 1 | 23027818 | G | A |
| 60 | EPHB2 | rs12027585 | 1 | 23028689 | C | T |
| 61 | EPHB2 | rs10753545 | 1 | 23030709 | A | G |
| 62 | EPHB2 | rs4655127 | 1 | 23039240 | A | G |
| 63 | EPHB2 | rs4655128 | 1 | 23042090 | C | T |
| 64 | EPHB2 | rs11800828 | 1 | 23044293 | A | G |
| 65 | EPHB2 | rs4655130 | 1 | 23048581 | A | G |
| 66 | EPHB2 | rs4654824 | 1 | 23055717 | C | T |
| 67 | EPHB2 | rs7413155 | 1 | 23056793 | C | A |
| 68 | EPHB2 | rs6670405 | 1 | 23061710 | T | C |
| 69 | EPHB2 | rs4655134 | 1 | 23071206 | T | G |
| 70 | EPHB2 | rs12131925 | 1 | 23071631 | C | T |
| 71 | EPHB2 | rs10799771 | 1 | 23079271 | A | G |
| 72 | EPHB2 | rs309499 | 1 | 23107533 | C | T |
| 73 | NTNG1 | rs4481881 | 1 | 107487941 | T | C |
| 74 | NTNG1 | rs4307594 | 1 | 107494690 | T | C |
| 75 | NTNG1 | rs17505688 | 1 | 107500564 | T | C |
| 76 | NTNG1 | rs12743478 | 1 | 107502629 | C | T |
| 77 | NTNG1 | rs4364907 | 1 | 107505396 | T | C |
| 78 | NTNG1 | rs9628993 | 1 | 107511243 | A | G |
| 79 | NTNG1 | rs10494061 | 1 | 107511901 | C | T |
| 80 | NTNG1 | rs6659943 | 1 | 107516001 | G | A |
| 81 | NTNG1 | rs4287207 | 1 | 107516477 | T | C |
| 82 | NTNG1 | rs4385741 | 1 | 107525842 | T | G |
| 83 | NTNG1 | rs10881456 | 1 | 107528015 | C | T |
| 84 | NTNG1 | rs12122485 | 1 | 107538207 | C | T |
| 85 | NTNG1 | rs4915025 | 1 | 107545507 | T | C |
| 86 | NTNG1 | rs6673296 | 1 | 107549799 | A | — |
| 87 | NTNG1 | rs4915029 | 1 | 107550427 | T | C |
| 88 | NTNG1 | rs10159449 | 1 | 107577827 | A | G |
| 89 | NTNG1 | rs12146111 | 1 | 107586887 | C | T |
| 90 | NTNG1 | rs10494066 | 1 | 107591031 | A | G |
| 91 | NTNG1 | rs3125685 | 1 | 107607205 | G | A |
| 92 | NTNG1 | rs3125677 | 1 | 107616418 | T | C |
| 93 | NTNG1 | rs4244126 | 1 | 107618274 | A | G |
| 94 | NTNG1 | rs12133394 | 1 | 107619073 | C | T |
| 95 | NTNG1 | rs3125673 | 1 | 107623708 | G | A |
| 96 | NTNG1 | rs7517024 | 1 | 107625466 | C | T |
| 97 | NTNG1 | rs3123376 | 1 | 107627389 | A | G |
| 98 | NTNG1 | rs7533102 | 1 | 107630473 | C | T |
| 99 | NTNG1 | rs10494067 | 1 | 107632381 | A | C |
| 100 | NTNG1 | rs4604720 | 1 | 107638682 | G | A |
| 101 | NTNG1 | rs4132604 | 1 | 107644362 | G | T |
| 102 | NTNG1 | rs10494068 | 1 | 107646033 | G | A |
| 103 | NTNG1 | rs4244127 | 1 | 107650563 | A | G |
| 104 | NTNG1 | rs6692538 | 1 | 107657726 | G | A |
| 105 | NTNG1 | rs7411507 | 1 | 107667501 | A | G |
| 106 | NTNG1 | rs11185090 | 1 | 107670247 | C | T |
| 107 | NTNG1 | rs7555412 | 1 | 107674459 | G | A |
| 108 | NTNG1 | rs4603156 | 1 | 107675338 | A | G |
| 109 | NTNG1 | rs6672549 | 1 | 107676482 | A | G |
| 110 | NTNG1 | rs12066815 | 1 | 107684566 | A | G |
| 111 | NTNG1 | rs7555385 | 1 | 107691171 | T | G |
| 112 | NTNG1 | rs17509795 | 1 | 107700129 | G | A |
| 113 | NTNG1 | rs12406016 | 1 | 107706631 | T | G |
| 114 | NTNG1 | rs6664221 | 1 | 107711401 | T | C |
| 115 | NTNG1 | rs11185100 | 1 | 107719842 | T | G |
| 116 | NTNG1 | rs6662274 | 1 | 107720889 | C | A |
| 117 | NTNG1 | rs12736002 | 1 | 107725673 | T | C |
| 118 | NTNG1 | rs6696776 | 1 | 107731728 | C | T |
| 119 | NTNG1 | rs6681988 | 1 | 107731888 | T | C |
| 120 | NTNG1 | rs746603 | 1 | 107732683 | T | C |
| 121 | NTNG1 | rs17014199 | 1 | 107736656 | G | A |
| 122 | NTNG1 | rs2587895 | 1 | 107737916 | C | T |
| 123 | NTNG1 | rs920835 | 1 | 107748325 | G | A |
| 124 | NTNG1 | rs12078289 | 1 | 107760492 | T | C |
| 125 | NTNG1 | rs12731224 | 1 | 107762682 | A | G |
| 126 | NTNG1 | rs600647 | 1 | 107773544 | G | A |
| 127 | NTNG1 | rs1373337 | 1 | 107781930 | G | A |
| 128 | NTNG1 | rs1444038 | 1 | 107782839 | G | A |
| 129 | NTNG1 | rs11185115 | 1 | 107784515 | G | A |
| 130 | NTNG1 | rs490516 | 1 | 107784870 | T | — |
| 131 | NTNG1 | rs12735678 | 1 | 107785658 | T | C |
| 132 | NTNG1 | rs536124 | 1 | 107789336 | A | G |
| 133 | NTNG1 | rs670053 | 1 | 107789741 | G | — |
| 134 | NTNG1 | rs11803905 | 1 | 107790837 | C | T |
| 135 | NTNG1 | rs640554 | 1 | 107791588 | C | T |
| 136 | NTNG1 | rs628117 | 1 | 107798629 | A | G |
| 137 | NTNG1 | rs597258 | 1 | 107800938 | T | — |
| 138 | NTNG1 | rs521721 | 1 | 107802971 | C | T |
| 139 | NTNG1 | rs6667549 | 1 | 107807146 | A | G |
| 140 | NTNG1 | rs977799 | 1 | 107807376 | C | T |
| 141 | NTNG1 | rs10881468 | 1 | 107812162 | T | G |
| 142 | NTNG1 | rs12354155 | 1 | 107812743 | G | A |
| 143 | NTNG1 | rs3813218 | 1 | 107817815 | G | T |
| 144 | NTNG1 | rs3813219 | 1 | 107817854 | T | C |
| 145 | NTNG1 | rs96501 | 1 | 107820370 | T | C |
| 146 | NTNG1 | rs594165 | 1 | 107820487 | C | A |
| 147 | NTNG1 | rs507203 | 1 | 107821720 | T | C |
| 148 | NTNG1 | rs4915045 | 1 | 107825112 | A | C |
| 149 | NTNG1 | rs597332 | 1 | 107826391 | G | A |
| 150 | NTNG1 | rs17019212 | 1 | 107830078 | T | C |
| 151 | GNAI3 | rs518076 | 1 | 109910562 | A | G |
| 152 | GNAI3 | rs17024043 | 1 | 109931928 | A | G |
| 153 | GNAI3 | rs7537507 | 1 | 109932628 | G | A |
| 154 | GNAI3 | rs541320 | 1 | 109933621 | A | G |
| 155 | GNAI3 | rs2483920 | 1 | 109935295 | G | T |
| 156 | GNAI3 | rs2301230 | 1 | 109937981 | C | T |
| 157 | GNAI3 | rs505303 | 1 | 109942500 | C | T |
| 158 | GNAI3 | rs1889470 | 1 | 109942944 | A | G |
| 159 | NRAS | rs969273 | 1 | 115058192 | T | C |
| 160 | NRAS | rs8453 | 1 | 115061122 | C | A |
| 161 | NRAS | rs10489525 | 1 | 115065208 | G | A |
| 162 | NRAS | rs6692870 | 1 | 115065314 | T | C |
| 163 | SEMA6C | rs4971004 | 1 | 149370702 | C | T |
| 164 | SEMA6C | rs4971007 | 1 | 149374761 | G | T |
| 165 | SEMA6C | rs11807526 | 1 | 149382621 | A | C |
| 166 | SEMA6C | rs12089658 | 1 | 149383634 | G | A |
| 167 | SEMA6C | rs7554936 | 1 | 149389113 | T | C |
| 168 | EFNA4 | rs12023499 | 1 | 153298000 | C | T |
| 169 | EFNA4 | rs4845710 | 1 | 153299638 | G | A |
| 170 | EFNA4 | rs11264305 | 1 | 153300196 | G | A |
| 171 | EFNA4 | rs3806256 | 1 | 153302235 | C | T |
| 172 | EFNA4 | rs4045192 | 1 | 153305380 | A | G |
| 173 | EFNA4 | rs11264306 | 1 | 153309949 | A | C |
| 174 | EFNA3 | rs3766916 | 1 | 153316026 | C | T |
| 175 | EFNA3 | rs1462855 | 1 | 153320343 | T | C |
| 176 | EFNA3 | rs2306124 | 1 | 153326475 | G | A |
| 177 | EFNA1 | rs9297 | 1 | 153373174 | A | G |
| 178 | SEMA4A | rs12128066 | 1 | 154393992 | T | C |
| 179 | SEMA4A | rs510441 | 1 | 154396592 | A | G |
| 180 | SEMA4A | rs1984508 | 1 | 154398241 | C | T |
| 181 | SEMA4A | rs11264452 | 1 | 154407556 | G | T |
| 182 | SEMA4A | rs12401573 | 1 | 154412842 | T | C |
| 183 | SEMA4A | rs7695 | 1 | 154413950 | T | C |
| 184 | SRGAP2 | rs12568232 | 1 | 204650485 | G | — |
| 185 | SRGAP2 | rs17006055 | 1 | 204655317 | C | T |
| 186 | SRGAP2 | rs12072062 | 1 | 204661633 | C | T |
| 187 | SRGAP2 | rs2244510 | 1 | 204661692 | T | C |
| 188 | SRGAP2 | rs12098229 | 1 | 204662187 | A | C |
| 189 | SRGAP2 | rs11120040 | 1 | 204667949 | C | T |
| 190 | SRGAP2 | rs2297539 | 1 | 204670158 | A | G |
| 191 | SRGAP2 | rs4845104 | 1 | 204675943 | C | T |
| 192 | SRGAP2 | rs2483064 | 1 | 204681395 | T | C |
| 193 | SRGAP2 | rs2483062 | 1 | 204684305 | C | T |
| 194 | SRGAP2 | rs2336938 | 1 | 204685422 | A | C |
| 195 | SRGAP2 | rs12129635 | 1 | 204687961 | G | T |
| 196 | SRGAP2 | rs2483061 | 1 | 204689619 | C | T |
| 197 | SRGAP2 | rs2483060 | 1 | 204690575 | G | A |
| 198 | SRGAP2 | rs12130290 | 1 | 204693800 | G | A |
| 199 | SRGAP2 | rs3813982 | 1 | 204696297 | T | C |
| 200 | SRGAP2 | rs7549189 | 1 | 204701918 | T | G |
| 201 | SRGAP2 | rs2990590 | 1 | 204702003 | T | C |
| 202 | PLXNA2 | rs2767567 | 1 | 206262704 | G | A |
| 203 | PLXNA2 | rs591752 | 1 | 206264408 | G | A |
| 204 | PLXNA2 | rs3811383 | 1 | 206270460 | G | A |
| 205 | PLXNA2 | rs1409831 | 1 | 206276460 | G | T |
| 206 | PLXNA2 | rs2767566 | 1 | 206279020 | T | C |
| 207 | PLXNA2 | rs2785622 | 1 | 206281127 | T | C |
| 208 | PLXNA2 | rs7514550 | 1 | 206288271 | T | G |
| 209 | PLXNA2 | rs6540451 | 1 | 206291980 | G | A |
| 210 | PLXNA2 | rs841865 | 1 | 206292532 | G | A |
| 211 | PLXNA2 | rs9803859 | 1 | 206297054 | T | C |
| 212 | PLXNA2 | rs12057548 | 1 | 206302787 | G | A |
| 213 | PLXNA2 | rs752016 | 1 | 206304300 | T | C |
| 214 | PLXNA2 | rs12057794 | 1 | 206306142 | C | A |
| 215 | PLXNA2 | rs12094094 | 1 | 206309981 | G | T |
| 216 | PLXNA2 | rs2478816 | 1 | 206311476 | C | T |
| 217 | PLXNA2 | rs2274446 | 1 | 206319079 | G | A |
| 218 | PLXNA2 | rs7540179 | 1 | 206320506 | G | A |
| 219 | PLXNA2 | rs716461 | 1 | 206324252 | G | A |
| 220 | PLXNA2 | rs2498024 | 1 | 206329773 | G | A |
| 221 | PLXNA2 | rs2498023 | 1 | 206329845 | T | G |
| 222 | PLXNA2 | rs2148699 | 1 | 206334201 | A | G |
| 223 | PLXNA2 | rs2249852 | 1 | 206334597 | A | G |
| 224 | PLXNA2 | rs12083980 | 1 | 206345500 | C | T |
| 225 | PLXNA2 | rs11118986 | 1 | 206345851 | G | A |
| 226 | PLXNA2 | rs4844642 | 1 | 206351974 | G | A |
| 227 | PLXNA2 | rs11118988 | 1 | 206362706 | A | G |
| 228 | PLXNA2 | rs6660047 | 1 | 206363721 | G | A |
| 229 | PLXNA2 | rs6540454 | 1 | 206364862 | G | A |
| 230 | PLXNA2 | rs681241 | 1 | 206367114 | T | G |
| 231 | PLXNA2 | rs895241 | 1 | 206369256 | T | C |
| 232 | PLXNA2 | rs3811390 | 1 | 206370676 | G | A |
| 233 | PLXNA2 | rs17187133 | 1 | 206373103 | T | G |
| 234 | PLXNA2 | rs17187140 | 1 | 206376398 | G | A |
| 235 | PLXNA2 | rs473500 | 1 | 206377532 | C | T |
| 236 | PLXNA2 | rs668878 | 1 | 206379933 | T | C |
| 237 | PLXNA2 | rs1875431 | 1 | 206387741 | C | T |
| 238 | PLXNA2 | rs11578181 | 1 | 206389763 | T | G |
| 239 | PLXNA2 | rs4844411 | 1 | 206392835 | C | T |
| 240 | PLXNA2 | rs550948 | 1 | 206394335 | C | T |
| 241 | PLXNA2 | rs493194 | 1 | 206394685 | G | A |
| 242 | PLXNA2 | rs664188 | 1 | 206394720 | T | C |
| 243 | PLXNA2 | rs2590688 | 1 | 206394847 | C | T |
| 244 | PLXNA2 | rs1604364 | 1 | 206396391 | A | G |
| 245 | PLXNA2 | rs510965 | 1 | 206397937 | C | T |
| 246 | PLXNA2 | rs11118996 | 1 | 206398272 | C | A |
| 247 | PLXNA2 | rs2256712 | 1 | 206404030 | G | A |
| 248 | PLXNA2 | rs2440900 | 1 | 206405591 | A | G |
| 249 | PLXNA2 | rs2590684 | 1 | 206405996 | C | A |
| 250 | PLXNA2 | rs11118997 | 1 | 206406224 | A | G |
| 251 | PLXNA2 | rs1249875 | 1 | 206408996 | A | G |
| 252 | PLXNA2 | rs6656560 | 1 | 206427770 | G | A |
| 253 | PLXNA2 | rs7411544 | 1 | 206428793 | G | T |
| 254 | PLXNA2 | rs12023768 | 1 | 206432924 | C | A |
| 255 | PLXNA2 | rs1609841 | 1 | 206434237 | C | T |
| 256 | PLXNA2 | rs11119011 | 1 | 206440018 | A | G |
| 257 | PLXNA2 | rs10863703 | 1 | 206442361 | T | C |
| 258 | PLXNA2 | rs1664232 | 1 | 206448663 | C | T |
| 259 | PLXNA2 | rs10863704 | 1 | 206448779 | C | T |
| 260 | PLXNA2 | rs1318276 | 1 | 206449632 | G | A |
| 261 | PLXNA2 | rs3748735 | 1 | 206457092 | C | T |
| 262 | PLXNA2 | rs881758 | 1 | 206460171 | C | A |
| 263 | PLXNA2 | rs3748737 | 1 | 206462779 | T | C |
| 264 | PLXNA2 | rs12126061 | 1 | 206462807 | G | A |
| 265 | PLXNA2 | rs1262046 | 1 | 206467115 | T | C |
| 266 | PLXNA2 | rs12566264 | 1 | 206473621 | C | T |
| 267 | PLXNA2 | rs2782934 | 1 | 206475126 | A | G |
| 268 | PLXNA2 | rs6685511 | 1 | 206476783 | G | A |
| 269 | PLXNA2 | rs12739500 | 1 | 206477133 | T | C |
| 270 | PLXNA2 | rs1272623 | 1 | 206477258 | A | C |
| 271 | PLXNA2 | rs1620298 | 1 | 206485911 | G | A |
| 272 | ITGB1 | rs7900442 | 10 | 33226550 | G | A |
| 273 | ITGB1 | rs2488314 | 10 | 33227470 | A | G |
| 274 | ITGB1 | rs1342548 | 10 | 33228108 | A | G |
| 275 | ITGB1 | rs2153875 | 10 | 33230573 | A | C |
| 276 | ITGB1 | rs2488320 | 10 | 33238917 | C | T |
| 277 | ITGB1 | rs4587680 | 10 | 33240788 | C | T |
| 278 | ITGB1 | rs2488326 | 10 | 33247503 | C | T |
| 279 | ITGB1 | rs3780873 | 10 | 33253686 | C | T |
| 280 | ITGB1 | rs10763902 | 10 | 33258082 | T | C |
| 281 | ITGB1 | rs2504001 | 10 | 33264893 | G | A |
| 282 | ITGB1 | rs10827164 | 10 | 33272634 | T | C |
| 283 | ITGB1 | rs2488336 | 10 | 33276311 | C | T |
| 284 | ITGB1 | rs1009002 | 10 | 33276907 | T | C |
| 285 | ITGB1 | rs1187070 | 10 | 33281144 | C | A |
| 286 | ITGB1 | rs11009157 | 10 | 33282407 | G | A |
| 287 | ITGB1 | rs1187078 | 10 | 33289139 | A | G |
| 288 | NRP1 | rs7099537 | 10 | 33502584 | A | G |
| 289 | NRP1 | rs7912484 | 10 | 33503607 | T | C |
| 290 | NRP1 | rs2506135 | 10 | 33505043 | T | C |
| 291 | NRP1 | rs1044268 | 10 | 33506572 | G | A |
| 292 | NRP1 | rs2506144 | 10 | 33508462 | C | T |
| 293 | NRP1 | rs2506145 | 10 | 33511136 | T | C |
| 294 | NRP1 | rs2228638 | 10 | 33515288 | G | A |
| 295 | NRP1 | rs734186 | 10 | 33524835 | C | T |
| 296 | NRP1 | rs2284944 | 10 | 33530847 | G | A |
| 297 | NRP1 | rs2250706 | 10 | 33533313 | G | A |
| 298 | NRP1 | rs2474714 | 10 | 33535989 | T | C |
| 299 | NRP1 | rs2506155 | 10 | 33543185 | C | A |
| 300 | NRP1 | rs2254826 | 10 | 33544471 | C | A |
| 301 | NRP1 | rs2269101 | 10 | 33546185 | G | A |
| 302 | NRP1 | rs1411923 | 10 | 33546784 | C | T |
| 303 | NRP1 | rs2269096 | 10 | 33548148 | G | A |
| 304 | NRP1 | rs2273466 | 10 | 33551053 | A | G |
| 305 | NRP1 | rs1331317 | 10 | 33557653 | T | C |
| 306 | NRP1 | rs11009311 | 10 | 33560360 | C | A |
| 307 | NRP1 | rs2269091 | 10 | 33569034 | C | A |
| 308 | NRP1 | rs2269088 | 10 | 33570056 | T | C |
| 309 | NRP1 | rs2269087 | 10 | 33570091 | G | A |
| 310 | NRP1 | rs17413155 | 10 | 33572761 | T | G |
| 311 | NRP1 | rs17413169 | 10 | 33573154 | T | C |
| 312 | NRP1 | rs10490939 | 10 | 33574886 | C | T |
| 313 | NRP1 | rs1888689 | 10 | 33575892 | T | G |
| 314 | NRP1 | rs1888688 | 10 | 33575913 | G | A |
| 315 | NRP1 | rs12243943 | 10 | 33576807 | G | A |
| 316 | NRP1 | rs11009323 | 10 | 33578627 | C | T |
| 317 | NRP1 | rs2383987 | 10 | 33579334 | T | C |
| 318 | NRP1 | rs11598565 | 10 | 33580454 | A | G |
| 319 | NRP1 | rs4934583 | 10 | 33583708 | A | G |
| 320 | NRP1 | rs1319013 | 10 | 33583935 | C | A |
| 321 | NRP1 | rs7096016 | 10 | 33584222 | C | A |
| 322 | NRP1 | rs11593943 | 10 | 33585087 | C | T |
| 323 | NRP1 | rs3780869 | 10 | 33587471 | G | A |
| 324 | NRP1 | rs10490938 | 10 | 33588382 | G | A |
| 325 | NRP1 | rs16934292 | 10 | 33589474 | G | A |
| 326 | NRP1 | rs11598845 | 10 | 33589567 | T | C |
| 327 | NRP1 | rs2070296 | 10 | 33592701 | G | A |
| 328 | NRP1 | rs2073320 | 10 | 33593263 | C | T |
| 329 | NRP1 | rs11819443 | 10 | 33594248 | C | T |
| 330 | NRP1 | rs10827224 | 10 | 33597285 | A | C |
| 331 | NRP1 | rs17296436 | 10 | 33601566 | A | G |
| 332 | NRP1 | rs17296443 | 10 | 33601580 | C | T |
| 333 | NRP1 | rs10827227 | 10 | 33601794 | C | T |
| 334 | NRP1 | rs2243668 | 10 | 33603899 | G | A |
| 335 | NRP1 | rs12774340 | 10 | 33606942 | T | C |
| 336 | NRP1 | rs1952983 | 10 | 33608941 | C | A |
| 337 | NRP1 | rs869636 | 10 | 33612045 | T | C |
| 338 | NRP1 | rs7079372 | 10 | 33613812 | C | T |
| 339 | NRP1 | rs2776928 | 10 | 33614411 | T | C |
| 340 | NRP1 | rs1331326 | 10 | 33619896 | A | G |
| 341 | NRP1 | rs4934871 | 10 | 33623095 | G | A |
| 342 | NRP1 | rs6481844 | 10 | 33624137 | T | C |
| 343 | NRP1 | rs12573218 | 10 | 33627739 | C | T |
| 344 | NRP1 | rs2776932 | 10 | 33632412 | T | C |
| 345 | NRP1 | rs2065364 | 10 | 33634008 | G | A |
| 346 | NRP1 | rs2804492 | 10 | 33642052 | C | T |
| 347 | NRP1 | rs2804493 | 10 | 33645682 | G | A |
| 348 | NRP1 | rs2776937 | 10 | 33646195 | G | A |
| 349 | NRP1 | rs11009340 | 10 | 33649577 | G | T |
| 350 | NRP1 | rs4934597 | 10 | 33654727 | C | T |
| 351 | NRP1 | rs11009344 | 10 | 33656091 | G | A |
| 352 | NRP1 | rs1360457 | 10 | 33656935 | C | T |
| 353 | CXCL12 | rs10900029 | 10 | 44182226 | G | A |
| 354 | CXCL12 | rs266094 | 10 | 44184306 | C | T |
| 355 | CXCL12 | rs1065297 | 10 | 44185982 | T | C |
| 356 | CXCL12 | rs197452 | 10 | 44190246 | C | T |
| 357 | CXCL12 | rs266087 | 10 | 44191068 | G | A |
| 358 | CXCL12 | rs2297630 | 10 | 44191554 | G | A |
| 359 | CXCL12 | rs4948878 | 10 | 44194827 | A | G |
| 360 | CXCL12 | rs2839692 | 10 | 44194873 | T | C |
| 361 | CXCL12 | rs2839690 | 10 | 44195172 | T | C |
| 362 | CXCL12 | rs3780891 | 10 | 44198719 | G | A |
| 363 | UNC5B | rs16928524 | 10 | 72644677 | G | T |
| 364 | UNC5B | rs2244140 | 10 | 72652260 | T | C |
| 365 | UNC5B | rs875001 | 10 | 72654703 | T | C |
| 366 | UNC5B | rs12570384 | 10 | 72656175 | C | T |
| 367 | UNC5B | rs12781290 | 10 | 72661101 | G | A |
| 368 | UNC5B | rs7893421 | 10 | 72661391 | C | T |
| 369 | UNC5B | rs16928536 | 10 | 72667088 | C | T |
| 370 | UNC5B | rs10999739 | 10 | 72668240 | C | T |
| 371 | UNC5B | rs2894147 | 10 | 72670949 | A | G |
| 372 | UNC5B | rs7080150 | 10 | 72675943 | G | A |
| 373 | UNC5B | rs4746080 | 10 | 72676626 | C | T |
| 374 | UNC5B | rs7899034 | 10 | 72680070 | G | A |
| 375 | UNC5B | rs7907878 | 10 | 72682295 | G | A |
| 376 | UNC5B | rs7902476 | 10 | 72693742 | C | A |
| 377 | UNC5B | rs11598330 | 10 | 72704959 | G | T |
| 378 | UNC5B | rs16928594 | 10 | 72706905 | T | C |
| 379 | UNC5B | rs11593534 | 10 | 72713022 | T | C |
| 380 | UNC5B | rs4747146 | 10 | 72715543 | T | C |
| 381 | UNC5B | rs6480504 | 10 | 72716105 | G | A |
| 382 | UNC5B | rs2277259 | 10 | 72717696 | A | G |
| 383 | UNC5B | rs10762435 | 10 | 72717903 | G | A |
| 384 | UNC5B | rs16928637 | 10 | 72718018 | G | A |
| 385 | UNC5B | rs3740462 | 10 | 72720989 | A | G |
| 386 | UNC5B | rs1105172 | 10 | 72725971 | G | T |
| 387 | UNC5B | rs2275577 | 10 | 72732054 | G | T |
| 388 | PPP3CB | rs12644 | 10 | 74867057 | G | A |
| 389 | PPP3CB | rs3763679 | 10 | 74904832 | C | T |
| 390 | PPP3CB | rs7923276 | 10 | 74919617 | C | T |
| 391 | PPP3CB | rs3793663 | 10 | 74930189 | T | C |
| 392 | SLIT1 | rs12240946 | 10 | 98746803 | T | G |
| 393 | SLIT1 | rs12766020 | 10 | 98751399 | A | G |
| 394 | SLIT1 | rs4917753 | 10 | 98756580 | C | A |
| 395 | SLIT1 | rs11188985 | 10 | 98757832 | T | G |
| 396 | SLIT1 | rs12254525 | 10 | 98764092 | T | C |
| 397 | SLIT1 | rs11188989 | 10 | 98769654 | C | T |
| 398 | SLIT1 | rs12258328 | 10 | 98774587 | G | T |
| 399 | SLIT1 | rs7922865 | 10 | 98775766 | T | C |
| 400 | SLIT1 | rs17112311 | 10 | 98776648 | G | A |
| 401 | SLIT1 | rs7896883 | 10 | 98777344 | T | C |
| 402 | SLIT1 | rs7086186 | 10 | 98782415 | A | G |
| 403 | SLIT1 | rs11188994 | 10 | 98782510 | C | T |
| 404 | SLIT1 | rs12268044 | 10 | 98784605 | C | T |
| 405 | SLIT1 | rs2636770 | 10 | 98795602 | G | A |
| 406 | SLIT1 | rs7902871 | 10 | 98799693 | A | G |
| 407 | SLIT1 | rs902470 | 10 | 98800871 | G | A |
| 408 | SLIT1 | rs12269131 | 10 | 98803361 | A | C |
| 409 | SLIT1 | rs2817675 | 10 | 98806776 | C | T |
| 410 | SLIT1 | rs2817676 | 10 | 98809307 | G | A |
| 411 | SLIT1 | rs7924220 | 10 | 98810126 | A | G |
| 412 | SLIT1 | rs2306953 | 10 | 98810304 | G | A |
| 413 | SLIT1 | rs2817677 | 10 | 98811818 | A | G |
| 414 | SLIT1 | rs12773769 | 10 | 98822687 | A | G |
| 415 | SLIT1 | rs2817683 | 10 | 98828684 | C | T |
| 416 | SLIT1 | rs2817685 | 10 | 98835526 | A | G |
| 417 | SLIT1 | rs2805602 | 10 | 98838925 | G | — |
| 418 | SLIT1 | rs2817688 | 10 | 98840790 | A | G |
| 419 | SLIT1 | rs2861828 | 10 | 98844400 | A | G |
| 420 | SLIT1 | rs2279508 | 10 | 98852731 | A | G |
| 421 | SLIT1 | rs2279510 | 10 | 98853179 | C | T |
| 422 | SLIT1 | rs7098931 | 10 | 98855197 | C | T |
| 423 | SLIT1 | rs7894400 | 10 | 98855615 | C | T |
| 424 | SLIT1 | rs2817694 | 10 | 98856512 | G | A |
| 425 | SLIT1 | rs2817698 | 10 | 98861595 | A | G |
| 426 | SLIT1 | rs2817701 | 10 | 98864103 | A | G |
| 427 | SLIT1 | rs2636785 | 10 | 98865119 | T | C |
| 428 | SLIT1 | rs2636788 | 10 | 98866931 | A | G |
| 429 | SLIT1 | rs902475 | 10 | 98872415 | G | A |
| 430 | SLIT1 | rs2817708 | 10 | 98879703 | C | T |
| 431 | SLIT1 | rs2817713 | 10 | 98886769 | A | C |
| 432 | SLIT1 | rs2817649 | 10 | 98889145 | T | C |
| 433 | SLIT1 | rs17112452 | 10 | 98893384 | T | C |
| 434 | SLIT1 | rs2817656 | 10 | 98895213 | A | G |
| 435 | SLIT1 | rs11189013 | 10 | 98899301 | C | T |
| 436 | SLIT1 | rs2636809 | 10 | 98900753 | T | C |
| 437 | SLIT1 | rs17410931 | 10 | 98902166 | C | T |
| 438 | SLIT1 | rs2784920 | 10 | 98903618 | A | C |
| 439 | SLIT1 | rs17502307 | 10 | 98910830 | G | A |
| 440 | SLIT1 | rs3000208 | 10 | 98911054 | C | A |
| 441 | SLIT1 | rs902467 | 10 | 98913521 | A | G |
| 442 | SLIT1 | rs12360055 | 10 | 98914808 | C | T |
| 443 | SLIT1 | rs885333 | 10 | 98915613 | G | A |
| 444 | SLIT1 | rs12767098 | 10 | 98916828 | A | G |
| 445 | SLIT1 | rs7073919 | 10 | 98916951 | A | G |
| 446 | SLIT1 | rs4919077 | 10 | 98918561 | C | T |
| 447 | SLIT1 | rs2636804 | 10 | 98920051 | G | A |
| 448 | SLIT1 | rs2636802 | 10 | 98920382 | G | A |
| 449 | SLIT1 | rs7073112 | 10 | 98920414 | T | C |
| 450 | SLIT1 | rs2253224 | 10 | 98923034 | A | C |
| 451 | SLIT1 | rs2817666 | 10 | 98923843 | G | A |
| 452 | SLIT1 | rs11595301 | 10 | 98928458 | C | T |
| 453 | SLIT1 | rs3758587 | 10 | 98936234 | C | T |
| 454 | SLIT1 | rs7474809 | 10 | 98940467 | C | T |
| 455 | SEMA4G | rs11190780 | 10 | 102734366 | A | G |
| 456 | SEMA4G | rs2863095 | 10 | 102736493 | C | T |
| 457 | SEMA4G | rs3740484 | 10 | 102737353 | G | T |
| 458 | ABLIM1 | rs6585278 | 10 | 116176372 | C | T |
| 459 | ABLIM1 | rs11196739 | 10 | 116178475 | C | T |
| 460 | ABLIM1 | rs11812741 | 10 | 116179920 | G | A |
| 461 | ABLIM1 | rs10787530 | 10 | 116183907 | G | A |
| 462 | ABLIM1 | rs941853 | 10 | 116189165 | G | A |
| 463 | ABLIM1 | rs727532 | 10 | 116194766 | A | G |
| 464 | ABLIM1 | rs7080135 | 10 | 116204106 | A | C |
| 465 | ABLIM1 | rs11813036 | 10 | 116206531 | T | C |
| 466 | ABLIM1 | rs12268190 | 10 | 116208374 | C | T |
| 467 | ABLIM1 | rs10885570 | 10 | 116212157 | G | A |
| 468 | ABLIM1 | rs6585283 | 10 | 116217311 | A | C |
| 469 | ABLIM1 | rs4457666 | 10 | 116217444 | T | G |
| 470 | ABLIM1 | rs1535386 | 10 | 116223224 | A | G |
| 471 | ABLIM1 | rs11814528 | 10 | 116223814 | C | T |
| 472 | ABLIM1 | rs10510005 | 10 | 116229075 | C | T |
| 473 | ABLIM1 | rs2475224 | 10 | 116233842 | G | A |
| 474 | ABLIM1 | rs1080709 | 10 | 116235947 | A | G |
| 475 | ABLIM1 | rs2475228 | 10 | 116242855 | A | G |
| 476 | ABLIM1 | rs7910989 | 10 | 116247513 | C | T |
| 477 | ABLIM1 | rs17717312 | 10 | 116247895 | G | A |
| 478 | ABLIM1 | rs2475229 | 10 | 116248374 | G | T |
| 479 | ABLIM1 | rs2475230 | 10 | 116249920 | A | G |
| 480 | ABLIM1 | rs10510006 | 10 | 116250617 | A | C |
| 481 | ABLIM1 | rs17091953 | 10 | 116253478 | T | C |
| 482 | ABLIM1 | rs12765660 | 10 | 116258629 | C | A |
| 483 | ABLIM1 | rs2475233 | 10 | 116262976 | C | T |
| 484 | ABLIM1 | rs941852 | 10 | 116265087 | A | G |
| 485 | ABLIM1 | rs2497707 | 10 | 116267515 | G | A |
| 486 | ABLIM1 | rs11196775 | 10 | 116269013 | T | G |
| 487 | ABLIM1 | rs2497708 | 10 | 116271540 | A | C |
| 488 | ABLIM1 | rs2475239 | 10 | 116273746 | G | T |
| 489 | ABLIM1 | rs2497714 | 10 | 116282551 | G | A |
| 490 | ABLIM1 | rs7913199 | 10 | 116284487 | C | T |
| 491 | ABLIM1 | rs11196780 | 10 | 116286644 | G | A |
| 492 | ABLIM1 | rs10885575 | 10 | 116287287 | A | G |
| 493 | ABLIM1 | rs883768 | 10 | 116290948 | G | A |
| 494 | ABLIM1 | rs986212 | 10 | 116301521 | C | T |
| 495 | ABLIM1 | rs10749170 | 10 | 116302100 | T | C |
| 496 | ABLIM1 | rs2497730 | 10 | 116305099 | C | T |
| 497 | ABLIM1 | rs10787538 | 10 | 116305941 | C | A |
| 498 | ABLIM1 | rs2483548 | 10 | 116309006 | T | C |
| 499 | ABLIM1 | rs7923449 | 10 | 116313710 | G | A |
| 500 | ABLIM1 | rs2483567 | 10 | 116322760 | G | A |
| 501 | ABLIM1 | rs3808949 | 10 | 116326282 | A | C |
| 502 | ABLIM1 | rs2483578 | 10 | 116336214 | T | C |
| 503 | ABLIM1 | rs11196793 | 10 | 116336919 | C | T |
| 504 | ABLIM1 | rs2483580 | 10 | 116337328 | G | A |
| 505 | ABLIM1 | rs1413195 | 10 | 116342037 | G | A |
| 506 | ABLIM1 | rs2483583 | 10 | 116344186 | T | G |
| 507 | ABLIM1 | rs2497663 | 10 | 116346418 | G | A |
| 508 | ABLIM1 | rs7914325 | 10 | 116352310 | A | G |
| 509 | ABLIM1 | rs10787540 | 10 | 116354414 | A | G |
| 510 | ABLIM1 | rs2497666 | 10 | 116355031 | A | G |
| 511 | ABLIM1 | rs11196799 | 10 | 116355203 | T | C |
| 512 | ABLIM1 | rs11196802 | 10 | 116357719 | C | T |
| 513 | ABLIM1 | rs2483593 | 10 | 116359696 | A | G |
| 514 | ABLIM1 | rs2483597 | 10 | 116363012 | A | G |
| 515 | ABLIM1 | rs2497669 | 10 | 116365126 | C | — |
| 516 | ABLIM1 | rs2483603 | 10 | 116367952 | C | A |
| 517 | ABLIM1 | rs17719089 | 10 | 116373333 | G | T |
| 518 | ABLIM1 | rs11196810 | 10 | 116373609 | G | T |
| 519 | ABLIM1 | rs1333689 | 10 | 116380465 | G | A |
| 520 | ABLIM1 | rs11196826 | 10 | 116397396 | C | T |
| 521 | ABLIM1 | rs4752071 | 10 | 116398627 | G | T |
| 522 | ABLIM1 | rs10885591 | 10 | 116399903 | C | A |
| 523 | ABLIM1 | rs12570718 | 10 | 116406949 | C | T |
| 524 | ABLIM1 | rs17092237 | 10 | 116407002 | C | T |
| 525 | ABLIM1 | rs1333682 | 10 | 116407537 | T | C |
| 526 | ABLIM1 | rs7893317 | 10 | 116409311 | G | A |
| 527 | ABLIM1 | rs11196845 | 10 | 116411832 | T | C |
| 528 | ABLIM1 | rs11597188 | 10 | 116423337 | C | T |
| 529 | ABLIM1 | rs4751639 | 10 | 116424800 | C | T |
| 530 | ABLIM1 | rs11196865 | 10 | 116427164 | T | C |
| 531 | ABLIM1 | rs11196869 | 10 | 116432608 | G | A |
| 532 | RRAS2 | rs11023175 | 11 | 14259109 | C | T |
| 533 | RRAS2 | rs11023177 | 11 | 14263156 | G | A |
| 534 | RRAS2 | rs2970334 | 11 | 14285931 | G | A |
| 535 | RRAS2 | rs2915405 | 11 | 14289311 | A | G |
| 536 | RRAS2 | rs10832242 | 11 | 14297080 | T | C |
| 537 | RRAS2 | rs2915409 | 11 | 14312308 | T | C |
| 538 | RRAS2 | rs4756788 | 11 | 14318636 | T | C |
| 539 | RRAS2 | rs2915400 | 11 | 14321527 | C | T |
| 540 | RRAS2 | rs11023194 | 11 | 14326345 | T | C |
| 541 | LRRC4C | rs2927213 | 11 | 40088008 | A | G |
| 542 | LRRC4C | rs10837344 | 11 | 40090059 | T | C |
| 543 | LRRC4C | rs11035693 | 11 | 40091818 | G | A |
| 544 | LRRC4C | rs2953310 | 11 | 40094119 | T | C |
| 545 | LRRC4C | rs7943560 | 11 | 40098953 | A | G |
| 546 | LRRC4C | rs7930525 | 11 | 40099241 | C | T |
| 547 | LRRC4C | rs11035695 | 11 | 40099920 | G | A |
| 548 | LRRC4C | rs16934385 | 11 | 40104272 | T | C |
| 549 | LRRC4C | rs1381567 | 11 | 40106260 | T | C |
| 550 | LRRC4C | rs10837346 | 11 | 40106698 | C | T |
| 551 | LRRC4C | rs12281232 | 11 | 40111640 | T | G |
| 552 | LRRC4C | rs7936167 | 11 | 40112635 | T | C |
| 553 | LRRC4C | rs7952540 | 11 | 40118548 | G | A |
| 554 | LRRC4C | rs12786815 | 11 | 40122113 | G | T |
| 555 | LRRC4C | rs11035715 | 11 | 40129548 | G | T |
| 556 | LRRC4C | rs10160235 | 11 | 40131206 | C | T |
| 557 | LRRC4C | rs11035719 | 11 | 40137148 | A | C |
| 558 | LRRC4C | rs1551833 | 11 | 40137989 | C | T |
| 559 | LRRC4C | rs11035722 | 11 | 40145807 | A | G |
| 560 | LRRC4C | rs4623893 | 11 | 40148185 | T | C |
| 561 | LRRC4C | rs7131259 | 11 | 40148705 | A | — |
| 562 | LRRC4C | rs4429048 | 11 | 40149102 | G | A |
| 563 | LRRC4C | rs4237677 | 11 | 40149430 | A | C |
| 564 | LRRC4C | rs4074148 | 11 | 40152089 | G | A |
| 565 | LRRC4C | rs4076390 | 11 | 40152140 | A | G |
| 566 | LRRC4C | rs7926722 | 11 | 40152669 | T | C |
| 567 | LRRC4C | rs11035727 | 11 | 40154363 | T | C |
| 568 | LRRC4C | rs4756581 | 11 | 40157926 | C | T |
| 569 | LRRC4C | rs4319506 | 11 | 40161717 | T | C |
| 570 | LRRC4C | rs12362407 | 11 | 40161767 | A | G |
| 571 | LRRC4C | rs6485187 | 11 | 40162079 | A | C |
| 572 | LRRC4C | rs17477151 | 11 | 40163660 | G | A |
| 573 | LRRC4C | rs4075903 | 11 | 40164499 | G | A |
| 574 | LRRC4C | rs7930773 | 11 | 40166432 | G | A |
| 575 | LRRC4C | rs7924805 | 11 | 40167828 | C | T |
| 576 | LRRC4C | rs10734479 | 11 | 40168727 | G | T |
| 577 | LRRC4C | rs7942248 | 11 | 40168948 | T | C |
| 578 | LRRC4C | rs10768584 | 11 | 40173527 | A | G |
| 579 | LRRC4C | rs10768586 | 11 | 40174397 | G | A |
| 580 | LRRC4C | rs4073087 | 11 | 40176401 | A | G |
| 581 | LRRC4C | rs7106026 | 11 | 40177990 | A | C |
| 582 | LRRC4C | rs7106166 | 11 | 40178083 | C | A |
| 583 | LRRC4C | rs4509742 | 11 | 40180711 | G | A |
| 584 | LRRC4C | rs10837366 | 11 | 40180925 | T | C |
| 585 | LRRC4C | rs11035737 | 11 | 40183780 | C | T |
| 586 | LRRC4C | rs12575363 | 11 | 40185227 | A | G |
| 587 | LRRC4C | rs16934480 | 11 | 40203061 | A | G |
| 588 | LRRC4C | rs7933522 | 11 | 40203678 | T | C |
| 589 | LRRC4C | rs1377109 | 11 | 40216948 | C | T |
| 590 | LRRC4C | rs1377108 | 11 | 40218702 | C | A |
| 591 | LRRC4C | rs7125624 | 11 | 40224673 | T | C |
| 592 | LRRC4C | rs16934616 | 11 | 40228018 | G | A |
| 593 | LRRC4C | rs1452465 | 11 | 40231509 | G | A |
| 594 | LRRC4C | rs1037412 | 11 | 40233301 | C | T |
| 595 | LRRC4C | rs999004 | 11 | 40234464 | T | C |
| 596 | LRRC4C | rs1020580 | 11 | 40236939 | A | G |
| 597 | LRRC4C | rs1020579 | 11 | 40237012 | T | C |
| 598 | LRRC4C | rs16934661 | 11 | 40243736 | A | C |
| 599 | LRRC4C | rs12577986 | 11 | 40252115 | G | A |
| 600 | LRRC4C | rs16934668 | 11 | 40254886 | T | C |
| 601 | LRRC4C | rs16934671 | 11 | 40254923 | T | — |
| 602 | LRRC4C | rs11035761 | 11 | 40255710 | G | A |
| 603 | LRRC4C | rs1901583 | 11 | 40257769 | G | A |
| 604 | LRRC4C | rs10501227 | 11 | 40262733 | A | G |
| 605 | LRRC4C | rs10837378 | 11 | 40263464 | A | G |
| 606 | LRRC4C | rs998447 | 11 | 40267365 | A | C |
| 607 | CFL1 | rs635375 | 11 | 65378571 | A | G |
| 608 | RHOD | rs7112925 | 11 | 66582736 | C | T |
| 609 | RHOD | rs11227678 | 11 | 66599045 | G | A |
| 610 | PAK1 | rs1377470 | 11 | 76728362 | A | C |
| 611 | PAK1 | rs1453342 | 11 | 76746418 | C | A |
| 612 | PAK1 | rs2602474 | 11 | 76750178 | G | A |
| 613 | PAK1 | rs12287077 | 11 | 76763694 | G | — |
| 614 | PAK1 | rs2729760 | 11 | 76766995 | T | C |
| 615 | PAK1 | rs2729747 | 11 | 76776127 | A | G |
| 616 | PAK1 | rs2729749 | 11 | 76776618 | G | A |
| 617 | PAK1 | rs495889 | 11 | 76796364 | T | C |
| 618 | PAK1 | rs2725830 | 11 | 76819366 | A | G |
| 619 | PAK1 | rs527589 | 11 | 76832287 | A | G |
| 620 | PAK1 | rs2852394 | 11 | 76833499 | G | T |
| 621 | ARHGEF12 | rs4938800 | 11 | 119714596 | G | A |
| 622 | ARHGEF12 | rs7932658 | 11 | 119716422 | G | A |
| 623 | ARHGEF12 | rs11217837 | 11 | 119737976 | A | G |
| 624 | ARHGEF12 | rs632137 | 11 | 119752243 | G | A |
| 625 | ARHGEF12 | rs1892894 | 11 | 119759894 | C | T |
| 626 | ARHGEF12 | rs622373 | 11 | 119762055 | T | C |
| 627 | ARHGEF12 | rs7933086 | 11 | 119774518 | G | A |
| 628 | ARHGEF12 | rs11217861 | 11 | 119793270 | C | T |
| 629 | ARHGEF12 | rs747419 | 11 | 119818749 | G | A |
| 630 | ARHGEF12 | rs894839 | 11 | 119842915 | A | G |
| 631 | ARHGEF12 | rs11217878 | 11 | 119845593 | G | A |
| 632 | ARHGEF12 | rs673974 | 11 | 119848014 | G | A |
| 633 | ARHGEF12 | rs2276035 | 11 | 119851570 | C | T |
| 634 | ARHGEF12 | rs503473 | 11 | 119860440 | A | G |
| 635 | ARHGEF12 | rs12364480 | 11 | 119870238 | T | C |
| 636 | ROBO3 | rs5017048 | 11 | 124239681 | G | T |
| 637 | ROBO3 | rs7102701 | 11 | 124242492 | T | C |
| 638 | ROBO3 | rs4936957 | 11 | 124245198 | C | T |
| 639 | ROBO3 | rs7925879 | 11 | 124245901 | G | A |
| 640 | ROBO3 | rs11219821 | 11 | 124246982 | T | C |
| 641 | ROBO3 | rs4606490 | 11 | 124247941 | T | C |
| 642 | KRAS | rs4963857 | 12 | 25247667 | G | A |
| 643 | KRAS | rs12321334 | 12 | 25248947 | T | C |
| 644 | KRAS | rs13096 | 12 | 25251108 | G | A |
| 645 | KRAS | rs9266 | 12 | 25253484 | C | T |
| 646 | KRAS | rs4246229 | 12 | 25258936 | A | G |
| 647 | KRAS | rs12579073 | 12 | 25267066 | A | C |
| 648 | KRAS | rs11047901 | 12 | 25267596 | G | A |
| 649 | KRAS | rs10842513 | 12 | 25275925 | C | T |
| 650 | KRAS | rs11047912 | 12 | 25277177 | T | C |
| 651 | KRAS | rs17388587 | 12 | 25280487 | A | G |
| 652 | KRAS | rs6487464 | 12 | 25282723 | C | T |
| 653 | KRAS | rs10842514 | 12 | 25283549 | C | T |
| 654 | KRAS | rs10842515 | 12 | 25284790 | T | C |
| 655 | KRAS | rs17388893 | 12 | 25285132 | A | C |
| 656 | KRAS | rs12813551 | 12 | 25286046 | T | C |
| 657 | KRAS | rs2970532 | 12 | 25287302 | C | T |
| 658 | KRAS | rs17389103 | 12 | 25288549 | T | C |
| 659 | KRAS | rs11047919 | 12 | 25290388 | C | A |
| 660 | KRAS | rs4368021 | 12 | 25293089 | T | C |
| 661 | KRAS | rs11047921 | 12 | 25299314 | C | A |
| 662 | KRAS | rs12320328 | 12 | 25299731 | A | G |
| 663 | RND1 | rs2453481 | 12 | 47534881 | C | T |
| 664 | RND1 | rs2453488 | 12 | 47541475 | C | T |
| 665 | RND1 | rs3782360 | 12 | 47543059 | G | A |
| 666 | RND1 | rs2446971 | 12 | 47548660 | A | C |
| 667 | SRGAP1 | rs17099873 | 12 | 62526269 | A | G |
| 668 | SRGAP1 | rs11175189 | 12 | 62532342 | A | C |
| 669 | SRGAP1 | rs12229174 | 12 | 62532933 | G | A |
| 670 | SRGAP1 | rs7304340 | 12 | 62549195 | T | C |
| 671 | SRGAP1 | rs11175194 | 12 | 62551104 | G | A |
| 672 | SRGAP1 | rs7966445 | 12 | 62552194 | A | G |
| 673 | SRGAP1 | rs7953208 | 12 | 62554272 | C | T |
| 674 | SRGAP1 | rs10878092 | 12 | 62560947 | G | T |
| 675 | SRGAP1 | rs17099889 | 12 | 62568240 | T | C |
| 676 | SRGAP1 | rs4346062 | 12 | 62570550 | A | G |
| 677 | SRGAP1 | rs11175204 | 12 | 62576824 | G | A |
| 678 | SRGAP1 | rs2335882 | 12 | 62593703 | C | T |
| 679 | SRGAP1 | rs12422918 | 12 | 62598877 | G | T |
| 680 | SRGAP1 | rs11175208 | 12 | 62602704 | T | C |
| 681 | SRGAP1 | rs9668434 | 12 | 62605000 | G | T |
| 682 | SRGAP1 | rs11175209 | 12 | 62609140 | T | C |
| 683 | SRGAP1 | rs11175211 | 12 | 62611921 | G | A |
| 684 | SRGAP1 | rs1989584 | 12 | 62619479 | G | T |
| 685 | SRGAP1 | rs17099944 | 12 | 62621845 | A | G |
| 686 | SRGAP1 | rs10784374 | 12 | 62622045 | A | G |
| 687 | SRGAP1 | rs17099949 | 12 | 62623072 | C | T |
| 688 | SRGAP1 | rs10878096 | 12 | 62627372 | A | G |
| 689 | SRGAP1 | rs1350136 | 12 | 62637173 | G | A |
| 690 | SRGAP1 | rs10878098 | 12 | 62638358 | G | A |
| 691 | SRGAP1 | rs12227635 | 12 | 62644742 | G | A |
| 692 | SRGAP1 | rs11175217 | 12 | 62645285 | C | T |
| 693 | SRGAP1 | rs11175219 | 12 | 62648986 | C | T |
| 694 | SRGAP1 | rs7300256 | 12 | 62652675 | C | T |
| 695 | SRGAP1 | rs3741607 | 12 | 62669937 | T | C |
| 696 | SRGAP1 | rs4516057 | 12 | 62685998 | T | C |
| 697 | SRGAP1 | rs1350137 | 12 | 62687679 | A | — |
| 698 | SRGAP1 | rs3930087 | 12 | 62688777 | G | A |
| 699 | SRGAP1 | rs7311377 | 12 | 62690201 | G | T |
| 700 | SRGAP1 | rs6581522 | 12 | 62701297 | A | G |
| 701 | SRGAP1 | rs17100009 | 12 | 62704085 | T | — |
| 702 | SRGAP1 | rs6421210 | 12 | 62704167 | C | T |
| 703 | SRGAP1 | rs6581525 | 12 | 62707815 | A | G |
| 704 | SRGAP1 | rs6581527 | 12 | 62708262 | A | G |
| 705 | SRGAP1 | rs11175239 | 12 | 62723275 | G | A |
| 706 | SRGAP1 | rs11175240 | 12 | 62728702 | C | A |
| 707 | SRGAP1 | rs4763135 | 12 | 62733222 | G | T |
| 708 | SRGAP1 | rs990309 | 12 | 62734563 | T | C |
| 709 | SRGAP1 | rs1162789 | 12 | 62736175 | G | A |
| 710 | SRGAP1 | rs17100060 | 12 | 62739380 | A | G |
| 711 | SRGAP1 | rs789715 | 12 | 62740794 | C | — |
| 712 | SRGAP1 | rs1827073 | 12 | 62744468 | T | G |
| 713 | SRGAP1 | rs17711374 | 12 | 62747578 | T | C |
| 714 | SRGAP1 | rs9652019 | 12 | 62751576 | C | A |
| 715 | SRGAP1 | rs10878103 | 12 | 62755030 | G | A |
| 716 | SRGAP1 | rs7980267 | 12 | 62770300 | T | C |
| 717 | SRGAP1 | rs1874314 | 12 | 62770981 | C | T |
| 718 | SRGAP1 | rs789744 | 12 | 62771627 | G | A |
| 719 | SRGAP1 | rs7960270 | 12 | 62772552 | T | C |
| 720 | SRGAP1 | rs1567765 | 12 | 62773694 | C | T |
| 721 | SRGAP1 | rs11835401 | 12 | 62787795 | G | A |
| 722 | SRGAP1 | rs789722 | 12 | 62806073 | C | T |
| 723 | SRGAP1 | rs7969810 | 12 | 62824645 | G | A |
| 724 | SRGAP1 | rs10748000 | 12 | 62826499 | C | T |
| 725 | SRGAP1 | rs7131691 | 12 | 62827227 | G | A |
| 726 | PLXNC1 | rs4761585 | 12 | 93064806 | T | C |
| 727 | PLXNC1 | rs4761586 | 12 | 93065046 | A | G |
| 728 | PLXNC1 | rs2230754 | 12 | 93067637 | C | T |
| 729 | PLXNC1 | rs3847799 | 12 | 93070029 | C | A |
| 730 | PLXNC1 | rs7131826 | 12 | 93070451 | T | C |
| 731 | PLXNC1 | rs10745676 | 12 | 93071666 | G | A |
| 732 | PLXNC1 | rs4237889 | 12 | 93073105 | G | A |
| 733 | PLXNC1 | rs17298648 | 12 | 93073681 | C | T |
| 734 | PLXNC1 | rs3847801 | 12 | 93074139 | T | C |
| 735 | PLXNC1 | rs11107420 | 12 | 93078944 | G | A |
| 736 | PLXNC1 | rs3843641 | 12 | 93083378 | C | T |
| 737 | PLXNC1 | rs10732654 | 12 | 93086551 | G | A |
| 738 | PLXNC1 | rs2291324 | 12 | 93086822 | C | T |
| 739 | PLXNC1 | rs3843642 | 12 | 93087769 | T | C |
| 740 | PLXNC1 | rs2365736 | 12 | 93088049 | T | C |
| 741 | PLXNC1 | rs3847804 | 12 | 93090429 | A | G |
| 742 | PLXNC1 | rs4761466 | 12 | 93093491 | G | T |
| 743 | PLXNC1 | rs4761590 | 12 | 93097942 | T | C |
| 744 | PLXNC1 | rs11107432 | 12 | 93101912 | G | T |
| 745 | PLXNC1 | rs11107433 | 12 | 93102828 | G | A |
| 746 | PLXNC1 | rs3847809 | 12 | 93103065 | A | G |
| 747 | PLXNC1 | rs3847810 | 12 | 93103156 | A | G |
| 748 | PLXNC1 | rs3751189 | 12 | 93104417 | A | G |
| 749 | PLXNC1 | rs769283 | 12 | 93104568 | A | G |
| 750 | PLXNC1 | rs17299268 | 12 | 93109247 | G | A |
| 751 | PLXNC1 | rs17022148 | 12 | 93109705 | A | G |
| 752 | PLXNC1 | rs10777585 | 12 | 93110844 | G | A |
| 753 | PLXNC1 | rs17022166 | 12 | 93111536 | A | C |
| 754 | PLXNC1 | rs10745679 | 12 | 93113118 | G | A |
| 755 | PLXNC1 | rs12425752 | 12 | 93117093 | C | T |
| 756 | PLXNC1 | rs10507031 | 12 | 93117123 | T | C |
| 757 | PLXNC1 | rs10859684 | 12 | 93118690 | C | T |
| 758 | PLXNC1 | rs10859685 | 12 | 93118768 | G | A |
| 759 | PLXNC1 | rs7137116 | 12 | 93119252 | T | G |
| 760 | PLXNC1 | rs7296806 | 12 | 93120689 | C | T |
| 761 | PLXNC1 | rs7298875 | 12 | 93126036 | C | T |
| 762 | PLXNC1 | rs2305971 | 12 | 93127431 | T | C |
| 763 | PLXNC1 | rs10507033 | 12 | 93129263 | T | C |
| 764 | PLXNC1 | rs7298918 | 12 | 93135143 | T | C |
| 765 | PLXNC1 | rs7969106 | 12 | 93136883 | A | C |
| 766 | PLXNC1 | rs11107458 | 12 | 93140116 | C | T |
| 767 | PLXNC1 | rs17022254 | 12 | 93142427 | C | T |
| 768 | PLXNC1 | rs11107461 | 12 | 93143158 | C | T |
| 769 | PLXNC1 | rs2291333 | 12 | 93145189 | A | G |
| 770 | PLXNC1 | rs2291332 | 12 | 93145269 | C | T |
| 771 | PLXNC1 | rs12099862 | 12 | 93146088 | A | G |
| 772 | PLXNC1 | rs7307839 | 12 | 93147873 | G | A |
| 773 | PLXNC1 | rs12314236 | 12 | 93148644 | T | C |
| 774 | PLXNC1 | rs10745681 | 12 | 93148834 | G | T |
| 775 | PLXNC1 | rs9943825 | 12 | 93149803 | T | C |
| 776 | PLXNC1 | rs864106 | 12 | 93161688 | A | G |
| 777 | PLXNC1 | rs870034 | 12 | 93164226 | C | T |
| 778 | PLXNC1 | rs2054724 | 12 | 93166996 | G | T |
| 779 | PLXNC1 | rs2291326 | 12 | 93173076 | A | G |
| 780 | PLXNC1 | rs865569 | 12 | 93173234 | C | T |
| 781 | PLXNC1 | rs2242499 | 12 | 93174456 | G | A |
| 782 | PLXNC1 | rs11107485 | 12 | 93176484 | T | C |
| 783 | PLXNC1 | rs2306664 | 12 | 93177358 | G | A |
| 784 | PLXNC1 | rs10507035 | 12 | 93179117 | G | A |
| 785 | PLXNC1 | rs11107489 | 12 | 93185176 | G | A |
| 786 | PLXNC1 | rs10507036 | 12 | 93186878 | G | A |
| 787 | PLXNC1 | rs7970004 | 12 | 93189313 | C | T |
| 788 | PLXNC1 | rs7957161 | 12 | 93189627 | T | C |
| 789 | PLXNC1 | rs12579612 | 12 | 93189924 | A | G |
| 790 | PLXNC1 | rs1175500 | 12 | 93189939 | A | G |
| 791 | PLXNC1 | rs7968586 | 12 | 93194229 | A | G |
| 792 | PLXNC1 | rs17302157 | 12 | 93194335 | G | T |
| 793 | PLXNC1 | rs4761601 | 12 | 93196133 | G | A |
| 794 | PLXNC1 | rs832504 | 12 | 93197019 | T | C |
| 795 | PLXNC1 | rs832502 | 12 | 93199273 | T | C |
| 796 | PLXNC1 | rs10777588 | 12 | 93202503 | C | T |
| 797 | PLXNC1 | rs7302972 | 12 | 93206738 | G | A |
| 798 | PLXNC1 | rs4761470 | 12 | 93207335 | G | A |
| 799 | PLXNC1 | rs10745682 | 12 | 93212041 | C | T |
| 800 | PLXNC1 | rs17302436 | 12 | 93217382 | T | C |
| 801 | NTN4 | rs7959749 | 12 | 94575308 | T | C |
| 802 | NTN4 | rs8699 | 12 | 94576014 | T | C |
| 803 | NTN4 | rs1052651 | 12 | 94576852 | C | T |
| 804 | NTN4 | rs2367563 | 12 | 94581315 | T | C |
| 805 | NTN4 | rs1421436 | 12 | 94583259 | C | T |
| 806 | NTN4 | rs728661 | 12 | 94595451 | T | C |
| 807 | NTN4 | rs11108193 | 12 | 94598454 | C | T |
| 808 | NTN4 | rs1362972 | 12 | 94599424 | G | T |
| 809 | NTN4 | rs2468369 | 12 | 94607998 | T | C |
| 810 | NTN4 | rs2431014 | 12 | 94608311 | T | C |
| 811 | NTN4 | rs17024532 | 12 | 94610881 | G | A |
| 812 | NTN4 | rs2468366 | 12 | 94612000 | G | A |
| 813 | NTN4 | rs7301446 | 12 | 94612579 | T | C |
| 814 | NTN4 | rs1362966 | 12 | 94617489 | C | A |
| 815 | NTN4 | rs10507058 | 12 | 94622878 | C | T |
| 816 | NTN4 | rs2468358 | 12 | 94623607 | A | G |
| 817 | NTN4 | rs11108203 | 12 | 94624725 | C | T |
| 818 | NTN4 | rs10507059 | 12 | 94629965 | C | T |
| 819 | NTN4 | rs4129599 | 12 | 94631074 | A | G |
| 820 | NTN4 | rs7300004 | 12 | 94633934 | G | A |
| 821 | NTN4 | rs10777720 | 12 | 94634522 | C | T |
| 822 | NTN4 | rs17287370 | 12 | 94635249 | A | G |
| 823 | NTN4 | rs11108211 | 12 | 94638160 | G | A |
| 824 | NTN4 | rs17287608 | 12 | 94643147 | A | C |
| 825 | NTN4 | rs4128893 | 12 | 94647740 | C | T |
| 826 | NTN4 | rs17024626 | 12 | 94648678 | A | G |
| 827 | NTN4 | rs11108221 | 12 | 94648978 | C | A |
| 828 | NTN4 | rs10507060 | 12 | 94653358 | T | C |
| 829 | NTN4 | rs7137835 | 12 | 94654538 | C | T |
| 830 | NTN4 | rs11108225 | 12 | 94654844 | C | T |
| 831 | NTN4 | rs17288108 | 12 | 94656026 | A | G |
| 832 | NTN4 | rs10444523 | 12 | 94656231 | A | G |
| 833 | NTN4 | rs6538669 | 12 | 94656727 | A | G |
| 834 | NTN4 | rs7310388 | 12 | 94679760 | C | T |
| 835 | NTN4 | rs10735333 | 12 | 94682959 | G | A |
| 836 | NTN4 | rs11612530 | 12 | 94683790 | C | T |
| 837 | NTN4 | rs10859938 | 12 | 94684307 | C | T |
| 838 | NTN4 | rs17041209 | 12 | 94684987 | C | T |
| 839 | NTN4 | rs10859940 | 12 | 94685116 | T | C |
| 840 | NTN4 | rs4762617 | 12 | 94686199 | A | C |
| 841 | NTN4 | rs4237913 | 12 | 94686419 | T | C |
| 842 | NTN4 | rs17024669 | 12 | 94688291 | G | A |
| 843 | NTN4 | rs4533108 | 12 | 94692536 | A | G |
| 844 | NTN4 | rs4432089 | 12 | 94701319 | C | T |
| 845 | NTN4 | rs7975425 | 12 | 94705996 | G | A |
| 846 | NTN4 | rs2117915 | 12 | 94709873 | T | C |
| 847 | EFNB2 | rs1407584 | 13 | 105937791 | C | A |
| 848 | EFNB2 | rs7322914 | 13 | 105938911 | C | T |
| 849 | EFNB2 | rs9520088 | 13 | 105947685 | T | C |
| 850 | EFNB2 | rs2391331 | 13 | 105955710 | T | C |
| 851 | EFNB2 | rs7983579 | 13 | 105957447 | T | G |
| 852 | EFNB2 | rs11840214 | 13 | 105960944 | A | G |
| 853 | EFNB2 | rs2391333 | 13 | 105964695 | C | T |
| 854 | EFNB2 | rs9520092 | 13 | 105965540 | T | C |
| 855 | EFNB2 | rs4772795 | 13 | 105967395 | A | G |
| 856 | EFNB2 | rs2391335 | 13 | 105969986 | G | T |
| 857 | EFNB2 | rs2893262 | 13 | 105970807 | G | A |
| 858 | EFNB2 | rs2391340 | 13 | 105971754 | G | T |
| 859 | EFNB2 | rs8000078 | 13 | 105977975 | G | T |
| 860 | NFATC4 | rs2228233 | 14 | 23909005 | C | T |
| 861 | NFATC4 | rs2295298 | 14 | 23913460 | G | A |
| 862 | NFATC4 | rs11628929 | 14 | 23913894 | G | A |
| 863 | NFATC4 | rs3742517 | 14 | 23917791 | T | C |
| 864 | NFATC4 | rs8008583 | 14 | 23918967 | C | T |
| 865 | NFATC4 | rs6573766 | 14 | 23921886 | T | G |
| 866 | CFL2 | rs9743965 | 14 | 34246481 | T | C |
| 867 | CFL2 | rs10132688 | 14 | 34254899 | T | C |
| 868 | PAK6 | rs2277562 | 15 | 38329491 | C | T |
| 869 | PAK6 | rs11638662 | 15 | 38333966 | C | T |
| 870 | PAK6 | rs2068001 | 15 | 38337441 | G | A |
| 871 | PAK6 | rs731497 | 15 | 38342357 | A | G |
| 872 | PAK6 | rs1075507 | 15 | 38343946 | G | A |
| 873 | PAK6 | rs936216 | 15 | 38344560 | T | G |
| 874 | PAK6 | rs6492939 | 15 | 38349182 | A | C |
| 875 | PAK6 | rs12905386 | 15 | 38349932 | G | A |
| 876 | PAK6 | rs936214 | 15 | 38352997 | C | T |
| 877 | PAK6 | rs2242119 | 15 | 38356115 | G | T |
| 878 | PAK6 | rs4924445 | 15 | 38360705 | G | A |
| 879 | CHP | rs6492995 | 15 | 39317004 | T | C |
| 880 | CHP | rs6492997 | 15 | 39328463 | G | A |
| 881 | CHP | rs8034073 | 15 | 39332905 | T | G |
| 882 | CHP | rs6492998 | 15 | 39333923 | A | G |
| 883 | CHP | rs8030098 | 15 | 39336240 | A | — |
| 884 | CHP | rs7175912 | 15 | 39342541 | T | C |
| 885 | CHP | rs16971726 | 15 | 39345329 | A | G |
| 886 | CHP | rs16971733 | 15 | 39349322 | C | A |
| 887 | CHP | rs16971734 | 15 | 39349685 | C | T |
| 888 | SEMA6D | rs1347891 | 15 | 45793870 | G | A |
| 889 | SEMA6D | rs586799 | 15 | 45812214 | A | G |
| 890 | SEMA6D | rs687566 | 15 | 45818573 | G | A |
| 891 | SEMA6D | rs1153820 | 15 | 45819258 | A | G |
| 892 | SEMA6D | rs765 | 15 | 45822977 | T | C |
| 893 | SEMA6D | rs76739 | 15 | 45829438 | A | G |
| 894 | SEMA6D | rs11070610 | 15 | 45831979 | C | T |
| 895 | SEMA6D | rs11855134 | 15 | 45834968 | A | G |
| 896 | SEMA6D | rs501916 | 15 | 45840521 | C | A |
| 897 | SEMA6D | rs3743276 | 15 | 45842411 | T | C |
| 898 | SEMA6D | rs3743279 | 15 | 45843511 | A | — |
| 899 | SEMA6D | rs3743281 | 15 | 45844250 | C | T |
| 900 | SEMA6D | rs532598 | 15 | 45845363 | C | T |
| 901 | SEMA6D | rs603569 | 15 | 45846221 | C | T |
| 902 | SEMA6D | rs8037600 | 15 | 45847623 | A | G |
| 903 | SEMA6D | rs568215 | 15 | 45850368 | G | A |
| 904 | SEMA6D | rs11857458 | 15 | 45855279 | A | G |
| 905 | SEMA6D | rs652856 | 15 | 45856459 | T | C |
| 906 | SEMA6D | rs8039913 | 15 | 45857783 | T | C |
| 907 | SEMA6D | rs9806697 | 15 | 45858085 | A | C |
| 908 | SEMA7A | rs2075594 | 15 | 72489714 | A | G |
| 909 | SEMA7A | rs5015024 | 15 | 72491641 | G | A |
| 910 | SEMA7A | rs885740 | 15 | 72492735 | C | T |
| 911 | SEMA7A | rs1046146 | 15 | 72493996 | G | A |
| 912 | SEMA7A | rs2075589 | 15 | 72497028 | C | T |
| 913 | SEMA7A | rs11857558 | 15 | 72500353 | T | C |
| 914 | SEMA7A | rs11852686 | 15 | 72502258 | T | C |
| 915 | SEMA7A | rs8036030 | 15 | 72503662 | G | A |
| 916 | SEMA4B | rs8036117 | 15 | 88561271 | T | C |
| 917 | SEMA4B | rs1543116 | 15 | 88561995 | A | G |
| 918 | SEMA4B | rs4932305 | 15 | 88565223 | G | A |
| 919 | SEMA4B | rs8030039 | 15 | 88568493 | C | T |
| 920 | SEMA4B | rs3751656 | 15 | 88572708 | C | T |
| 921 | SEMA4B | rs1044246 | 15 | 88573204 | G | A |
| 922 | SEMA4B | rs8033436 | 15 | 88574146 | G | A |
| 923 | FES | rs4702 | 15 | 89227564 | A | G |
| 924 | FES | rs1029420 | 15 | 89242090 | G | A |
| 925 | MAPK3 | rs11865086 | 16 | 30037994 | C | A |
| 926 | NFATC3 | rs9932251 | 16 | 66689233 | G | A |
| 927 | NFATC3 | rs7205935 | 16 | 66709658 | A | G |
| 928 | NFATC3 | rs2418736 | 16 | 66712363 | C | T |
| 929 | NFATC3 | rs7193701 | 16 | 66742661 | G | A |
| 930 | NFATC3 | rs4359427 | 16 | 66760248 | C | T |
| 931 | NFATC3 | rs12598 | 16 | 66783016 | G | A |
| 932 | NFATC3 | rs9931987 | 16 | 66814140 | G | A |
| 933 | NFATC3 | rs11871 | 16 | 66818935 | G | A |
| 934 | NFAT5 | rs2161630 | 16 | 68163962 | G | A |
| 935 | NFAT5 | rs12447326 | 16 | 68188354 | C | T |
| 936 | NFAT5 | rs33063 | 16 | 68197718 | G | A |
| 937 | NFAT5 | rs17297088 | 16 | 68198345 | C | T |
| 938 | NFAT5 | rs3848278 | 16 | 68198368 | T | C |
| 939 | NFAT5 | rs9936464 | 16 | 68201550 | C | A |
| 940 | NFAT5 | rs11861500 | 16 | 68205167 | A | — |
| 941 | NFAT5 | rs16958997 | 16 | 68209202 | A | G |
| 942 | NFAT5 | rs9924271 | 16 | 68237722 | A | G |
| 943 | NFAT5 | rs1437137 | 16 | 68262239 | G | A |
| 944 | NFAT5 | rs9889219 | 16 | 68278916 | G | A |
| 945 | NFAT5 | rs1064825 | 16 | 68281721 | G | A |
| 946 | EFNB3 | rs7141 | 17 | 7555326 | A | G |
| 947 | NTN1 | rs940853 | 17 | 8867899 | T | C |
| 948 | NTN1 | rs1473664 | 17 | 8869136 | C | A |
| 949 | NTN1 | rs1880646 | 17 | 8870570 | T | C |
| 950 | NTN1 | rs4791331 | 17 | 8872807 | C | T |
| 951 | NTN1 | rs7216356 | 17 | 8872957 | G | A |
| 952 | NTN1 | rs7223172 | 17 | 8873613 | C | A |
| 953 | NTN1 | rs8065243 | 17 | 8875062 | G | A |
| 954 | NTN1 | rs940854 | 17 | 8875488 | C | T |
| 955 | NTN1 | rs9674710 | 17 | 8881934 | G | A |
| 956 | NTN1 | rs10521156 | 17 | 8883455 | C | T |
| 957 | NTN1 | rs8076457 | 17 | 8884654 | C | T |
| 958 | NTN1 | rs9915089 | 17 | 8893619 | C | T |
| 959 | NTN1 | rs8069536 | 17 | 8897010 | G | T |
| 960 | NTN1 | rs4063993 | 17 | 8900405 | G | A |
| 961 | NTN1 | rs1525758 | 17 | 8901608 | G | A |
| 962 | NTN1 | rs8081823 | 17 | 8906276 | G | A |
| 963 | NTN1 | rs9303235 | 17 | 8907017 | A | G |
| 964 | NTN1 | rs2030795 | 17 | 8908001 | G | A |
| 965 | NTN1 | rs7222847 | 17 | 8908105 | T | C |
| 966 | NTN1 | rs12948329 | 17 | 8910382 | A | G |
| 967 | NTN1 | rs10852910 | 17 | 8914277 | G | A |
| 968 | NTN1 | rs17808998 | 17 | 8919071 | C | T |
| 969 | NTN1 | rs8079384 | 17 | 8922857 | G | A |
| 970 | NTN1 | rs869019 | 17 | 8925627 | G | A |
| 971 | NTN1 | rs9913468 | 17 | 8928575 | G | A |
| 972 | NTN1 | rs2030793 | 17 | 8931626 | G | T |
| 973 | NTN1 | rs1568901 | 17 | 8933807 | A | G |
| 974 | NTN1 | rs871827 | 17 | 8934626 | T | G |
| 975 | NTN1 | rs7207041 | 17 | 8936271 | C | A |
| 976 | NTN1 | rs9902550 | 17 | 8939490 | A | G |
| 977 | NTN1 | rs9893768 | 17 | 8942956 | A | C |
| 978 | NTN1 | rs4791779 | 17 | 8946118 | G | A |
| 979 | NTN1 | rs4791333 | 17 | 8950320 | G | A |
| 980 | NTN1 | rs9914560 | 17 | 8951168 | T | C |
| 981 | NTN1 | rs2091317 | 17 | 8953426 | C | T |
| 982 | NTN1 | rs4791334 | 17 | 8957733 | T | C |
| 983 | NTN1 | rs4641781 | 17 | 8966802 | G | A |
| 984 | NTN1 | rs4791794 | 17 | 8967535 | C | T |
| 985 | NTN1 | rs17809081 | 17 | 8969844 | G | A |
| 986 | NTN1 | rs4791337 | 17 | 8975450 | G | A |
| 987 | NTN1 | rs7211163 | 17 | 8975602 | G | A |
| 988 | NTN1 | rs4791796 | 17 | 8980297 | G | A |
| 989 | NTN1 | rs4791798 | 17 | 8987681 | G | A |
| 990 | NTN1 | rs1860283 | 17 | 8992042 | C | T |
| 991 | NTN1 | rs3826464 | 17 | 8998634 | G | A |
| 992 | NTN1 | rs9896508 | 17 | 8998654 | C | A |
| 993 | NTN1 | rs726598 | 17 | 9000213 | G | A |
| 994 | NTN1 | rs16957962 | 17 | 9000751 | A | G |
| 995 | NTN1 | rs4300689 | 17 | 9001690 | G | A |
| 996 | NTN1 | rs1008359 | 17 | 9001974 | C | T |
| 997 | NTN1 | rs16942842 | 17 | 9003212 | G | A |
| 998 | NTN1 | rs9896554 | 17 | 9005397 | G | A |
| 999 | NTN1 | rs2072321 | 17 | 9006726 | C | T |
| 1000 | NTN1 | rs4791811 | 17 | 9007578 | A | G |
| 1001 | NTN1 | rs9901968 | 17 | 9011371 | G | A |
| 1002 | NTN1 | rs1034893 | 17 | 9013280 | A | G |
| 1003 | NTN1 | rs758413 | 17 | 9014435 | C | T |
| 1004 | NTN1 | rs3785975 | 17 | 9019052 | C | T |
| 1005 | NTN1 | rs3785977 | 17 | 9021296 | G | A |
| 1006 | NTN1 | rs3785981 | 17 | 9025043 | G | T |
| 1007 | NTN1 | rs4791812 | 17 | 9025766 | C | T |
| 1008 | NTN1 | rs2302196 | 17 | 9026953 | C | A |
| 1009 | NTN1 | rs8067387 | 17 | 9028146 | G | A |
| 1010 | NTN1 | rs12602314 | 17 | 9029788 | T | C |
| 1011 | NTN1 | rs3785982 | 17 | 9030949 | C | T |
| 1012 | NTN1 | rs6503186 | 17 | 9032986 | T | C |
| 1013 | NTN1 | rs12945811 | 17 | 9035059 | T | C |
| 1014 | NTN1 | rs7225607 | 17 | 9036407 | G | A |
| 1015 | NTN1 | rs16958012 | 17 | 9036434 | A | G |
| 1016 | NTN1 | rs8081873 | 17 | 9038658 | T | G |
| 1017 | NTN1 | rs7221329 | 17 | 9038953 | C | T |
| 1018 | NTN1 | rs740642 | 17 | 9042894 | C | T |
| 1019 | NTN1 | rs3785983 | 17 | 9048959 | T | C |
| 1020 | NTN1 | rs16958048 | 17 | 9051741 | C | T |
| 1021 | NTN1 | rs7224773 | 17 | 9053828 | G | T |
| 1022 | NTN1 | rs718097 | 17 | 9056130 | A | G |
| 1023 | NTN1 | rs12940357 | 17 | 9061595 | G | A |
| 1024 | NTN1 | rs9899990 | 17 | 9062304 | G | A |
| 1025 | NTN1 | rs9897341 | 17 | 9064619 | T | C |
| 1026 | NTN1 | rs1009675 | 17 | 9065603 | T | G |
| 1027 | NTN1 | rs3785988 | 17 | 9065630 | G | A |
| 1028 | NTN1 | rs12939263 | 17 | 9067735 | C | T |
| 1029 | NTN1 | rs3785991 | 17 | 9071043 | G | A |
| 1030 | NTN1 | rs3785996 | 17 | 9073586 | G | A |
| 1031 | NTN1 | rs11652483 | 17 | 9075422 | G | A |
| 1032 | NTN1 | rs3786003 | 17 | 9077878 | G | A |
| 1033 | NTN1 | rs9909240 | 17 | 9080816 | A | G |
| 1034 | NTN1 | rs3744656 | 17 | 9082048 | T | G |
| 1035 | NTN1 | rs2277672 | 17 | 9086693 | A | G |
| 1036 | NTN1 | rs2302198 | 17 | 9088785 | G | A |
| 1037 | RAC3 | rs4969481 | 17 | 77586501 | C | T |
| 1038 | ROCK1 | rs8085654 | 18 | 16824233 | G | A |
| 1039 | ROCK1 | rs288980 | 18 | 16863578 | C | T |
| 1040 | ROCK1 | rs7239317 | 18 | 16864065 | C | T |
| 1041 | ROCK1 | rs17202361 | 18 | 16872068 | T | C |
| 1042 | ROCK1 | rs17202375 | 18 | 16875271 | A | G |
| 1043 | ROCK1 | rs7240577 | 18 | 16880540 | C | — |
| 1044 | ROCK1 | rs2127958 | 18 | 16907608 | T | C |
| 1045 | ROCK1 | rs1481280 | 18 | 16909449 | C | A |
| 1046 | ROCK1 | rs1006881 | 18 | 16935291 | G | A |
| 1047 | ROCK1 | rs11874761 | 18 | 16945764 | G | A |
| 1048 | DCC | rs17753970 | 18 | 48119269 | G | A |
| 1049 | DCC | rs2256 | 18 | 48128802 | C | A |
| 1050 | DCC | rs2195781 | 18 | 48130634 | C | T |
| 1051 | DCC | rs1443570 | 18 | 48132618 | T | C |
| 1052 | DCC | rs10502942 | 18 | 48132806 | C | T |
| 1053 | DCC | rs1443568 | 18 | 48134240 | C | T |
| 1054 | DCC | rs17816227 | 18 | 48135559 | A | G |
| 1055 | DCC | rs8088453 | 18 | 48138021 | C | T |
| 1056 | DCC | rs3862675 | 18 | 48139135 | T | C |
| 1057 | DCC | rs1443579 | 18 | 48141998 | G | A |
| 1058 | DCC | rs1443578 | 18 | 48142238 | C | T |
| 1059 | DCC | rs12455312 | 18 | 48142821 | G | A |
| 1060 | DCC | rs1431790 | 18 | 48146323 | C | T |
| 1061 | DCC | rs11875845 | 18 | 48150107 | C | T |
| 1062 | DCC | rs12969239 | 18 | 48152365 | T | C |
| 1063 | DCC | rs10502943 | 18 | 48154999 | A | C |
| 1064 | DCC | rs7227798 | 18 | 48158767 | G | A |
| 1065 | DCC | rs11660123 | 18 | 48159351 | A | G |
| 1066 | DCC | rs9967342 | 18 | 48161807 | T | C |
| 1067 | DCC | rs8093706 | 18 | 48167069 | A | C |
| 1068 | DCC | rs7231326 | 18 | 48169408 | G | A |
| 1069 | DCC | rs11877840 | 18 | 48171678 | T | C |
| 1070 | DCC | rs3862678 | 18 | 48176145 | G | A |
| 1071 | DCC | rs12456286 | 18 | 48180442 | T | C |
| 1072 | DCC | rs3862681 | 18 | 48184688 | T | C |
| 1073 | DCC | rs3915401 | 18 | 48188604 | G | T |
| 1074 | DCC | rs3862683 | 18 | 48189303 | T | C |
| 1075 | DCC | rs3862684 | 18 | 48191249 | G | A |
| 1076 | DCC | rs1560531 | 18 | 48191698 | T | C |
| 1077 | DCC | rs1222938 | 18 | 48195164 | A | G |
| 1078 | DCC | rs12960771 | 18 | 48195999 | T | C |
| 1079 | DCC | rs2960617 | 18 | 48202733 | A | C |
| 1080 | DCC | rs17748116 | 18 | 48203972 | C | A |
| 1081 | DCC | rs17816780 | 18 | 48207845 | C | T |
| 1082 | DCC | rs1145242 | 18 | 48209457 | T | C |
| 1083 | DCC | rs1367865 | 18 | 48215053 | G | A |
| 1084 | DCC | rs1619323 | 18 | 48215948 | T | C |
| 1085 | DCC | rs9304422 | 18 | 48216882 | C | T |
| 1086 | DCC | rs10502944 | 18 | 48217904 | A | — |
| 1087 | DCC | rs6508142 | 18 | 48218149 | G | T |
| 1088 | DCC | rs347547 | 18 | 48223956 | G | — |
| 1089 | DCC | rs17754840 | 18 | 48230457 | G | A |
| 1090 | DCC | rs347546 | 18 | 48231042 | C | T |
| 1091 | DCC | rs347542 | 18 | 48234610 | T | C |
| 1092 | DCC | rs347535 | 18 | 48238734 | T | C |
| 1093 | DCC | rs12607228 | 18 | 48242131 | T | C |
| 1094 | DCC | rs12456332 | 18 | 48244111 | A | G |
| 1095 | DCC | rs1221983 | 18 | 48247264 | C | A |
| 1096 | DCC | rs2119840 | 18 | 48261248 | C | T |
| 1097 | DCC | rs12457422 | 18 | 48263676 | C | — |
| 1098 | DCC | rs1221967 | 18 | 48264436 | A | G |
| 1099 | DCC | rs1145245 | 18 | 48271109 | A | C |
| 1100 | DCC | rs1221986 | 18 | 48272440 | G | A |
| 1101 | DCC | rs7238242 | 18 | 48276299 | G | A |
| 1102 | DCC | rs17828125 | 18 | 48280234 | A | G |
| 1103 | DCC | rs2168159 | 18 | 48280913 | G | A |
| 1104 | DCC | rs7243931 | 18 | 48284720 | A | G |
| 1105 | DCC | rs1144049 | 18 | 48284910 | T | C |
| 1106 | DCC | rs1144050 | 18 | 48285118 | G | T |
| 1107 | DCC | rs1031062 | 18 | 48292120 | A | C |
| 1108 | DCC | rs1037531 | 18 | 48292757 | G | — |
| 1109 | DCC | rs2045154 | 18 | 48293674 | A | C |
| 1110 | DCC | rs1302850 | 18 | 48297403 | T | G |
| 1111 | DCC | rs1301943 | 18 | 48297691 | A | G |
| 1112 | DCC | rs1144055 | 18 | 48300270 | A | C |
| 1113 | DCC | rs1144063 | 18 | 48302680 | A | C |
| 1114 | DCC | rs12964400 | 18 | 48306360 | A | G |
| 1115 | DCC | rs1144065 | 18 | 48309172 | A | C |
| 1116 | DCC | rs1816793 | 18 | 48310894 | C | T |
| 1117 | DCC | rs1465943 | 18 | 48315280 | G | A |
| 1118 | DCC | rs1975750 | 18 | 48316559 | A | G |
| 1119 | DCC | rs1454731 | 18 | 48334365 | A | G |
| 1120 | DCC | rs1144081 | 18 | 48341901 | T | C |
| 1121 | DCC | rs7231796 | 18 | 48342017 | C | A |
| 1122 | DCC | rs9946542 | 18 | 48354022 | A | G |
| 1123 | DCC | rs993927 | 18 | 48355292 | G | T |
| 1124 | DCC | rs993928 | 18 | 48355447 | C | — |
| 1125 | DCC | rs9959870 | 18 | 48356801 | G | A |
| 1126 | DCC | rs1143768 | 18 | 48361975 | C | T |
| 1127 | DCC | rs4940191 | 18 | 48362228 | G | A |
| 1128 | DCC | rs948590 | 18 | 48378926 | G | A |
| 1129 | DCC | rs1145299 | 18 | 48379680 | T | C |
| 1130 | DCC | rs1221872 | 18 | 48384335 | G | A |
| 1131 | DCC | rs1221874 | 18 | 48385108 | G | A |
| 1132 | DCC | rs1221875 | 18 | 48385267 | G | A |
| 1133 | DCC | rs17681706 | 18 | 48385281 | A | G |
| 1134 | DCC | rs1221877 | 18 | 48387824 | G | A |
| 1135 | DCC | rs1221883 | 18 | 48389263 | T | C |
| 1136 | DCC | rs1221884 | 18 | 48389344 | G | A |
| 1137 | DCC | rs1228469 | 18 | 48396423 | A | G |
| 1138 | DCC | rs1221864 | 18 | 48398531 | G | A |
| 1139 | DCC | rs2219556 | 18 | 48399088 | T | C |
| 1140 | DCC | rs1380650 | 18 | 48401043 | T | C |
| 1141 | DCC | rs1221866 | 18 | 48405477 | A | G |
| 1142 | DCC | rs1221868 | 18 | 48406227 | G | A |
| 1143 | DCC | rs1221869 | 18 | 48406960 | C | T |
| 1144 | DCC | rs1237775 | 18 | 48407572 | C | T |
| 1145 | DCC | rs4940196 | 18 | 48408404 | G | A |
| 1146 | DCC | rs12606567 | 18 | 48408860 | C | T |
| 1147 | DCC | rs1675225 | 18 | 48412076 | C | T |
| 1148 | DCC | rs7231528 | 18 | 48412118 | T | C |
| 1149 | DCC | rs11872911 | 18 | 48412843 | A | G |
| 1150 | DCC | rs12968691 | 18 | 48414828 | G | A |
| 1151 | DCC | rs12327170 | 18 | 48426999 | G | A |
| 1152 | DCC | rs17681801 | 18 | 48427619 | C | T |
| 1153 | DCC | rs10853620 | 18 | 48432166 | G | A |
| 1154 | DCC | rs12606713 | 18 | 48433842 | A | G |
| 1155 | DCC | rs11082932 | 18 | 48434137 | A | C |
| 1156 | DCC | rs4940199 | 18 | 48434702 | T | C |
| 1157 | DCC | rs1460191 | 18 | 48435190 | G | T |
| 1158 | DCC | rs1466373 | 18 | 48439208 | T | C |
| 1159 | DCC | rs17749090 | 18 | 48439455 | T | C |
| 1160 | DCC | rs999170 | 18 | 48439467 | G | A |
| 1161 | DCC | rs7239496 | 18 | 48440823 | A | G |
| 1162 | DCC | rs768204 | 18 | 48441871 | C | A |
| 1163 | DCC | rs4556886 | 18 | 48441980 | A | G |
| 1164 | DCC | rs9966348 | 18 | 48446700 | C | A |
| 1165 | DCC | rs17828903 | 18 | 48447955 | A | G |
| 1166 | DCC | rs7245154 | 18 | 48450415 | A | G |
| 1167 | DCC | rs4542757 | 18 | 48452722 | T | C |
| 1168 | DCC | rs1870580 | 18 | 48458402 | T | G |
| 1169 | DCC | rs17755888 | 18 | 48459799 | G | A |
| 1170 | DCC | rs1460200 | 18 | 48462960 | T | C |
| 1171 | DCC | rs4940206 | 18 | 48463794 | G | A |
| 1172 | DCC | rs12962177 | 18 | 48464878 | C | T |
| 1173 | DCC | rs1460199 | 18 | 48465385 | T | G |
| 1174 | DCC | rs8084407 | 18 | 48466067 | C | T |
| 1175 | DCC | rs1903276 | 18 | 48466258 | T | C |
| 1176 | DCC | rs1460198 | 18 | 48466647 | G | T |
| 1177 | DCC | rs1460196 | 18 | 48469410 | G | A |
| 1178 | DCC | rs7236766 | 18 | 48476794 | G | T |
| 1179 | DCC | rs16960544 | 18 | 48478896 | C | T |
| 1180 | DCC | rs16955409 | 18 | 48484955 | G | A |
| 1181 | DCC | rs1160400 | 18 | 48485283 | A | G |
| 1182 | DCC | rs898438 | 18 | 48485785 | A | G |
| 1183 | DCC | rs2054289 | 18 | 48492952 | G | A |
| 1184 | DCC | rs7227438 | 18 | 48494462 | T | — |
| 1185 | DCC | rs12966053 | 18 | 48498502 | G | A |
| 1186 | DCC | rs9966514 | 18 | 48502757 | C | T |
| 1187 | DCC | rs4641847 | 18 | 48503880 | A | C |
| 1188 | DCC | rs8089980 | 18 | 48504219 | T | C |
| 1189 | DCC | rs9958731 | 18 | 48505466 | T | C |
| 1190 | DCC | rs7230305 | 18 | 48505826 | T | — |
| 1191 | DCC | rs4939702 | 18 | 48512935 | G | A |
| 1192 | DCC | rs6508153 | 18 | 48516961 | C | A |
| 1193 | DCC | rs9304427 | 18 | 48519937 | C | T |
| 1194 | DCC | rs10083941 | 18 | 48536120 | C | T |
| 1195 | DCC | rs768048 | 18 | 48539396 | C | T |
| 1196 | DCC | rs8092051 | 18 | 48539910 | G | A |
| 1197 | DCC | rs6508159 | 18 | 48542018 | C | A |
| 1198 | DCC | rs1431743 | 18 | 48548289 | C | T |
| 1199 | DCC | rs1431742 | 18 | 48548676 | T | G |
| 1200 | DCC | rs4940211 | 18 | 48554389 | G | A |
| 1201 | DCC | rs9950339 | 18 | 48561250 | A | G |
| 1202 | DCC | rs17469388 | 18 | 48562130 | G | T |
| 1203 | DCC | rs10515956 | 18 | 48562430 | G | A |
| 1204 | DCC | rs10469016 | 18 | 48580883 | G | A |
| 1205 | DCC | rs4939705 | 18 | 48588362 | G | A |
| 1206 | DCC | rs1431730 | 18 | 48600949 | C | T |
| 1207 | DCC | rs2096994 | 18 | 48601487 | G | T |
| 1208 | DCC | rs12605728 | 18 | 48602574 | G | A |
| 1209 | DCC | rs9957838 | 18 | 48605652 | T | C |
| 1210 | DCC | rs7233856 | 18 | 48606628 | G | A |
| 1211 | DCC | rs9964026 | 18 | 48607209 | T | G |
| 1212 | DCC | rs16955617 | 18 | 48609015 | C | T |
| 1213 | DCC | rs9948007 | 18 | 48609805 | T | C |
| 1214 | DCC | rs965971 | 18 | 48610758 | G | A |
| 1215 | DCC | rs7240741 | 18 | 48611062 | G | T |
| 1216 | DCC | rs727514 | 18 | 48611438 | A | G |
| 1217 | DCC | rs11082952 | 18 | 48611793 | G | A |
| 1218 | DCC | rs2116378 | 18 | 48615582 | G | A |
| 1219 | DCC | rs10502952 | 18 | 48620939 | A | G |
| 1220 | DCC | rs1573411 | 18 | 48621410 | G | A |
| 1221 | DCC | rs7239852 | 18 | 48624918 | T | G |
| 1222 | DCC | rs7239877 | 18 | 48624970 | G | — |
| 1223 | DCC | rs8088779 | 18 | 48627805 | G | T |
| 1224 | DCC | rs1030594 | 18 | 48628028 | A | G |
| 1225 | DCC | rs1030593 | 18 | 48628153 | T | C |
| 1226 | DCC | rs7506794 | 18 | 48629590 | C | A |
| 1227 | DCC | rs2032256 | 18 | 48635204 | C | T |
| 1228 | DCC | rs6508 169 | 18 | 48635668 | A | C |
| 1229 | DCC | rs752870 | 18 | 48640333 | G | A |
| 1230 | DCC | rs11665486 | 18 | 48654444 | C | T |
| 1231 | DCC | rs1431748 | 18 | 48654506 | T | G |
| 1232 | DCC | rs9304435 | 18 | 48670227 | T | C |
| 1233 | DCC | rs1943108 | 18 | 48679020 | A | G |
| 1234 | DCC | rs1943107 | 18 | 48679130 | T | C |
| 1235 | DCC | rs1943093 | 18 | 48681222 | T | C |
| 1236 | DCC | rs2156283 | 18 | 48684273 | A | G |
| 1237 | DCC | rs1541287 | 18 | 48690659 | C | T |
| 1238 | DCC | rs9964756 | 18 | 48695815 | G | T |
| 1239 | DCC | rs1943097 | 18 | 48696064 | G | T |
| 1240 | DCC | rs12963047 | 18 | 48714549 | C | T |
| 1241 | DCC | rs1943132 | 18 | 48720895 | G | A |
| 1242 | DCC | rs7241707 | 18 | 48729008 | T | C |
| 1243 | DCC | rs4940230 | 18 | 48750682 | A | C |
| 1244 | DCC | rs9955796 | 18 | 48751104 | A | C |
| 1245 | DCC | rs6508181 | 18 | 48752353 | G | A |
| 1246 | DCC | rs9965121 | 18 | 48759638 | A | G |
| 1247 | DCC | rs8088528 | 18 | 48768382 | G | A |
| 1248 | DCC | rs7504990 | 18 | 48771774 | C | T |
| 1249 | DCC | rs7242428 | 18 | 48782880 | A | G |
| 1250 | DCC | rs9945571 | 18 | 48783169 | G | T |
| 1251 | DCC | rs9951405 | 18 | 48790602 | G | A |
| 1252 | DCC | rs7505465 | 18 | 48798869 | C | T |
| 1253 | DCC | rs4078288 | 18 | 48801249 | T | C |
| 1254 | DCC | rs9950125 | 18 | 48802542 | G | A |
| 1255 | DCC | rs4940235 | 18 | 48807971 | C | A |
| 1256 | DCC | rs7506242 | 18 | 48808725 | A | G |
| 1257 | DCC | rs8087273 | 18 | 48819257 | A | G |
| 1258 | DCC | rs6508191 | 18 | 48831704 | T | C |
| 1259 | DCC | rs4132357 | 18 | 48834973 | A | G |
| 1260 | DCC | rs11874659 | 18 | 48842258 | C | T |
| 1261 | DCC | rs4129439 | 18 | 48843081 | A | G |
| 1262 | DCC | rs9964201 | 18 | 48854550 | C | A |
| 1263 | DCC | rs4270245 | 18 | 48875048 | G | A |
| 1264 | DCC | rs10502963 | 18 | 48875453 | T | C |
| 1265 | DCC | rs9959803 | 18 | 48876902 | T | C |
| 1266 | DCC | rs4414555 | 18 | 48897810 | T | C |
| 1267 | DCC | rs1893368 | 18 | 48916171 | A | G |
| 1268 | DCC | rs8089828 | 18 | 48923723 | C | T |
| 1269 | DCC | rs8088857 | 18 | 48923782 | C | T |
| 1270 | DCC | rs8092993 | 18 | 48924109 | G | A |
| 1271 | DCC | rs4939723 | 18 | 48927087 | G | A |
| 1272 | DCC | rs10153414 | 18 | 48928770 | G | — |
| 1273 | DCC | rs4359514 | 18 | 48935108 | C | T |
| 1274 | DCC | rs9959914 | 18 | 48946456 | A | G |
| 1275 | DCC | rs6417121 | 18 | 48957744 | G | A |
| 1276 | DCC | rs10502962 | 18 | 48965412 | A | G |
| 1277 | DCC | rs7238728 | 18 | 48966745 | G | A |
| 1278 | DCC | rs3794923 | 18 | 48981516 | G | A |
| 1279 | DCC | rs11662175 | 18 | 48982985 | T | C |
| 1280 | DCC | rs11082972 | 18 | 48987148 | T | C |
| 1281 | DCC | rs12962705 | 18 | 48994377 | C | T |
| 1282 | DCC | rs7243444 | 18 | 48997412 | G | A |
| 1283 | DCC | rs4609919 | 18 | 48998837 | G | A |
| 1284 | DCC | rs6508210 | 18 | 49001189 | C | A |
| 1285 | DCC | rs10502966 | 18 | 49002497 | A | G |
| 1286 | DCC | rs7243756 | 18 | 49004873 | G | A |
| 1287 | DCC | rs8087057 | 18 | 49023407 | G | A |
| 1288 | DCC | rs2879526 | 18 | 49023541 | A | G |
| 1289 | DCC | rs11082974 | 18 | 49024169 | C | A |
| 1290 | DCC | rs1431197 | 18 | 49045958 | C | T |
| 1291 | DCC | rs10502967 | 18 | 49046088 | C | T |
| 1292 | DCC | rs16956462 | 18 | 49053520 | A | — |
| 1293 | DCC | rs1431187 | 18 | 49057974 | G | A |
| 1294 | DCC | rs1988344 | 18 | 49076736 | A | G |
| 1295 | DCC | rs6508218 | 18 | 49078003 | A | G |
| 1296 | DCC | rs4940251 | 18 | 49087080 | A | C |
| 1297 | DCC | rs8097413 | 18 | 49104729 | T | C |
| 1298 | DCC | rs8087757 | 18 | 49111739 | G | A |
| 1299 | DCC | rs11872471 | 18 | 49114530 | G | T |
| 1300 | DCC | rs12968567 | 18 | 49127466 | C | T |
| 1301 | DCC | rs11874922 | 18 | 49140166 | A | G |
| 1302 | DCC | rs10502969 | 18 | 49141125 | A | C |
| 1303 | DCC | rs10502970 | 18 | 49141839 | T | C |
| 1304 | DCC | rs8095929 | 18 | 49148198 | T | C |
| 1305 | DCC | rs8091068 | 18 | 49154430 | C | T |
| 1306 | DCC | rs11082983 | 18 | 49157825 | A | G |
| 1307 | DCC | rs1910713 | 18 | 49158637 | C | A |
| 1308 | DCC | rs1394466 | 18 | 49159204 | G | A |
| 1309 | DCC | rs11665242 | 18 | 49161125 | A | G |
| 1310 | DCC | rs12965919 | 18 | 49174634 | A | C |
| 1311 | DCC | rs1010360 | 18 | 49182439 | A | C |
| 1312 | DCC | rs11082993 | 18 | 49189351 | A | G |
| 1313 | DCC | rs10853631 | 18 | 49190778 | A | G |
| 1314 | DCC | rs11876154 | 18 | 49194256 | A | G |
| 1315 | DCC | rs10502976 | 18 | 49195960 | A | C |
| 1316 | DCC | rs12953529 | 18 | 49222971 | A | G |
| 1317 | DCC | rs12604940 | 18 | 49226213 | A | G |
| 1318 | DCC | rs7232617 | 18 | 49278804 | C | T |
| 1319 | DCC | rs1105642 | 18 | 49281085 | G | A |
| 1320 | DCC | rs870508 | 18 | 49281553 | T | G |
| 1321 | DCC | rs4453579 | 18 | 49301831 | G | A |
| 1322 | DCC | rs2270954 | 18 | 49311296 | C | A |
| 1323 | DCC | rs2292043 | 18 | 49313161 | T | C |
| 1324 | NFATC1 | rs9962479 | 18 | 75256172 | G | A |
| 1325 | NFATC1 | rs549752 | 18 | 75259213 | T | C |
| 1326 | NFATC1 | rs2958431 | 18 | 75261956 | C | T |
| 1327 | NFATC1 | rs2850724 | 18 | 75263701 | G | A |
| 1328 | NFATC1 | rs8090692 | 18 | 75264917 | A | G |
| 1329 | NFATC1 | rs8090864 | 18 | 75265044 | A | G |
| 1330 | NFATC1 | rs8091180 | 18 | 75265231 | A | G |
| 1331 | NFATC1 | rs304933 | 18 | 75265431 | G | A |
| 1332 | NFATC1 | rs1562724 | 18 | 75270559 | C | T |
| 1333 | NFATC1 | rs2230112 | 18 | 75272049 | G | T |
| 1334 | NFATC1 | rs1017860 | 18 | 75273194 | C | T |
| 1335 | NFATC1 | rs2002311 | 18 | 75274967 | T | C |
| 1336 | NFATC1 | rs4799055 | 18 | 75282991 | G | T |
| 1337 | NFATC1 | rs1562722 | 18 | 75288153 | A | G |
| 1338 | NFATC1 | rs11664725 | 18 | 75293104 | A | G |
| 1339 | NFATC1 | rs8097537 | 18 | 75294234 | C | T |
| 1340 | NFATC1 | rs11664153 | 18 | 75296801 | G | A |
| 1341 | NFATC1 | rs3826567 | 18 | 75300466 | C | T |
| 1342 | NFATC1 | rs306872 | 18 | 75301833 | T | C |
| 1343 | NFATC1 | rs3786194 | 18 | 75308112 | A | G |
| 1344 | NFATC1 | rs9966033 | 18 | 75312752 | T | C |
| 1345 | NFATC1 | rs8090560 | 18 | 75314143 | G | A |
| 1346 | NFATC1 | rs7236492 | 18 | 75321604 | C | T |
| 1347 | NFATC1 | rs8090312 | 18 | 75326142 | A | G |
| 1348 | NFATC1 | rs754505 | 18 | 75327356 | G | A |
| 1349 | NFATC1 | rs7227107 | 18 | 75328464 | A | G |
| 1350 | NFATC1 | rs160189 | 18 | 75333697 | A | G |
| 1351 | NFATC1 | rs3826569 | 18 | 75342653 | G | A |
| 1352 | NFATC1 | rs1667667 | 18 | 75345988 | A | G |
| 1353 | NFATC1 | rs396127 | 18 | 75351626 | T | C |
| 1354 | NFATC1 | rs1667670 | 18 | 75352049 | C | T |
| 1355 | NFATC1 | rs1667682 | 18 | 75359569 | C | T |
| 1356 | NFATC1 | rs12963519 | 18 | 75365109 | C | T |
| 1357 | NFATC1 | rs8099113 | 18 | 75367142 | G | A |
| 1358 | NFATC1 | rs372741 | 18 | 75370277 | T | C |
| 1359 | NFATC1 | rs177820 | 18 | 75377952 | T | C |
| 1360 | NFATC1 | rs12958819 | 18 | 75378393 | G | A |
| 1361 | NFATC1 | rs12959273 | 18 | 75378707 | G | A |
| 1362 | NFATC1 | rs2044750 | 18 | 75380738 | G | A |
| 1363 | NFATC1 | rs9948543 | 18 | 75387291 | A | G |
| 1364 | NFATC1 | rs11661539 | 18 | 75388784 | C | T |
| 1365 | NFATC1 | rs9518 | 18 | 75389794 | T | C |
| 1366 | NFATC1 | rs308526 | 18 | 75391014 | C | T |
| 1367 | NFATC1 | rs308527 | 18 | 75391097 | T | C |
| 1368 | NFATC1 | rs838050 | 18 | 75391290 | G | T |
| 1369 | NFATC1 | rs183374 | 18 | 75392406 | G | A |
| 1370 | EFNA2 | rs2379239 | 19 | 1234757 | C | T |
| 1371 | EFNA2 | rs1558130 | 19 | 1253265 | T | C |
| 1372 | SEMA6B | rs966384 | 19 | 4489599 | C | T |
| 1373 | SEMA6B | rs4806985 | 19 | 4491429 | G | A |
| 1374 | SEMA6B | rs4807602 | 19 | 4499015 | A | G |
| 1375 | SEMA6B | rs4807604 | 19 | 4504185 | A | G |
| 1376 | SEMA6B | rs1610093 | 19 | 4505774 | A | G |
| 1377 | SEMA6B | rs10853973 | 19 | 4506310 | C | T |
| 1378 | SEMA6B | rs10409783 | 19 | 4506786 | G | A |
| 1379 | SEMA6B | rs17434453 | 19 | 4508299 | C | T |
| 1380 | SEMA6B | rs16992439 | 19 | 4510474 | A | G |
| 1381 | SEMA6B | rs7249087 | 19 | 4510646 | G | A |
| 1382 | PAK4 | rs8106725 | 19 | 44303543 | A | G |
| 1383 | PAK4 | rs12609418 | 19 | 44309292 | C | T |
| 1384 | PAK4 | rs692191 | 19 | 44314686 | T | C |
| 1385 | PAK4 | rs11669124 | 19 | 44315092 | G | A |
| 1386 | PAK4 | rs17722300 | 19 | 44322375 | G | A |
| 1387 | PAK4 | rs11878648 | 19 | 44329879 | G | A |
| 1388 | PAK4 | rs691577 | 19 | 44348241 | G | A |
| 1389 | PAK4 | rs7257109 | 19 | 44354747 | G | A |
| 1390 | PAK4 | rs1529712 | 19 | 44358807 | T | C |
| 1391 | RRAS | rs8107595 | 19 | 54826532 | G | A |
| 1392 | RRAS | rs1865077 | 19 | 54831904 | T | C |
| 1393 | RRAS | rs12104272 | 19 | 54839864 | G | A |
| 1394 | ROCK2 | rs921322 | 2 | 11237111 | C | T |
| 1395 | ROCK2 | rs8996 | 2 | 11238328 | G | A |
| 1396 | ROCK2 | rs6753921 | 2 | 11244206 | G | A |
| 1397 | ROCK2 | rs1515219 | 2 | 11286529 | C | T |
| 1398 | ROCK2 | rs11884214 | 2 | 11287537 | C | A |
| 1399 | ROCK2 | rs13404430 | 2 | 11298564 | A | C |
| 1400 | ROCK2 | rs6716817 | 2 | 11303285 | G | A |
| 1401 | ROCK2 | rs12479227 | 2 | 11311804 | C | T |
| 1402 | ROCK2 | rs4669700 | 2 | 11318989 | T | C |
| 1403 | ROCK2 | rs10203916 | 2 | 11369435 | T | C |
| 1404 | ROCK2 | rs753770 | 2 | 11382973 | A | G |
| 1405 | ROCK2 | rs12622447 | 2 | 11392896 | G | A |
| 1406 | ROCK2 | rs7581184 | 2 | 11406982 | A | G |
| 1407 | DPYSL5 | rs562311 | 2 | 26951590 | C | T |
| 1408 | DPYSL5 | rs12615509 | 2 | 26952270 | G | A |
| 1409 | DPYSL5 | rs7596707 | 2 | 26954785 | C | T |
| 1410 | DPYSL5 | rs4665920 | 2 | 26982338 | G | A |
| 1411 | DPYSL5 | rs9309559 | 2 | 26995019 | A | C |
| 1412 | DPYSL5 | rs1371614 | 2 | 27006378 | C | T |
| 1413 | DPYSL5 | rs3769137 | 2 | 27022994 | C | T |
| 1414 | PPP3R1 | rs1868402 | 2 | 68262541 | T | C |
| 1415 | PPP3R1 | rs12994570 | 2 | 68266852 | G | T |
| 1416 | PPP3R1 | rs6758449 | 2 | 68290612 | G | A |
| 1417 | PPP3R1 | rs7560138 | 2 | 68328270 | A | G |
| 1418 | SEMA4F | rs205650 | 5 | 74738119 | A | G |
| 1419 | SEMA4F | rs11900406 | 2 | 74748856 | T | C |
| 1420 | SEMA4F | rs363608 | 2 | 74755110 | C | T |
| 1421 | SEMA4F | rs363640 | 2 | 74755736 | A | C |
| 1422 | SEMA4F | rs363641 | 2 | 74756098 | G | — |
| 1423 | SEMA4F | rs363611 | 2 | 74759708 | C | T |
| 1424 | SEMA4F | rs363612 | 2 | 74764581 | T | C |
| 1425 | SEMA4C | rs6576989 | 2 | 96888826 | T | C |
| 1426 | NCK2 | rs997522 | 2 | 105732664 | A | G |
| 1427 | NCK2 | rs12987484 | 2 | 105733993 | T | C |
| 1428 | NCK2 | rs12712216 | 2 | 105739103 | G | A |
| 1429 | NCK2 | rs13028719 | 2 | 105739601 | T | C |
| 1430 | NCK2 | rs1114882 | 2 | 105743001 | G | A |
| 1431 | NCK2 | rs4851843 | 2 | 105744773 | T | C |
| 1432 | NCK2 | rs11691815 | 2 | 105747875 | C | T |
| 1433 | NCK2 | rs4851848 | 2 | 105749723 | G | A |
| 1434 | NCK2 | rs4851857 | 2 | 105750949 | T | C |
| 1435 | NCK2 | rs12470376 | 2 | 105762810 | C | T |
| 1436 | NCK2 | rs1897169 | 2 | 105764056 | T | C |
| 1437 | NCK2 | rs7571132 | 2 | 105765345 | C | T |
| 1438 | NCK2 | rs6732799 | 2 | 105768386 | C | T |
| 1439 | NCK2 | rs2376977 | 2 | 105774523 | G | A |
| 1440 | NCK2 | rs7588033 | 2 | 105774665 | C | T |
| 1441 | NCK2 | rs9646959 | 2 | 105778623 | G | A |
| 1442 | NCK2 | rs2047699 | 2 | 105779509 | G | A |
| 1443 | NCK2 | rs12464067 | 2 | 105782544 | A | G |
| 1444 | NCK2 | rs1367406 | 2 | 105786835 | A | G |
| 1445 | NCK2 | rs12995333 | 2 | 105802798 | G | T |
| 1446 | NCK2 | rs4851870 | 2 | 105803688 | C | T |
| 1447 | NCK2 | rs1465639 | 2 | 105805613 | A | G |
| 1448 | NCK2 | rs2163350 | 2 | 105811713 | C | T |
| 1449 | NCK2 | rs7589561 | 2 | 105820517 | T | C |
| 1450 | NCK2 | rs2377339 | 2 | 105823723 | A | G |
| 1451 | NCK2 | rs2163349 | 2 | 105824080 | T | C |
| 1452 | NCK2 | rs6543342 | 2 | 105828688 | C | T |
| 1453 | NCK2 | rs10180659 | 2 | 105831805 | C | T |
| 1454 | NCK2 | rs3769504 | 2 | 105843524 | C | T |
| 1455 | NCK2 | rs3769503 | 2 | 105843717 | G | A |
| 1456 | NCK2 | rs3754806 | 2 | 105853814 | T | C |
| 1457 | NCK2 | rs879900 | 2 | 105860779 | T | C |
| 1458 | NCK2 | rs6740343 | 2 | 105871910 | G | A |
| 1459 | CXCR4 | rs16832740 | 2 | 136584346 | T | C |
| 1460 | EPHA4 | rs2069212 | 2 | 221986132 | C | T |
| 1461 | EPHA4 | rs3087584 | 2 | 221991525 | T | C |
| 1462 | EPHA4 | rs17370971 | 2 | 221992521 | C | T |
| 1463 | EPHA4 | rs3770208 | 2 | 221992890 | G | A |
| 1464 | EPHA4 | rs10498110 | 2 | 221996113 | T | C |
| 1465 | EPHA4 | rs16862636 | 2 | 221996624 | T | C |
| 1466 | EPHA4 | rs2303901 | 2 | 221999146 | T | C |
| 1467 | EPHA4 | rs10211091 | 2 | 222002512 | A | G |
| 1468 | EPHA4 | rs3770205 | 2 | 222004004 | G | T |
| 1469 | EPHA4 | rs4340507 | 2 | 222005351 | C | T |
| 1470 | EPHA4 | rs3770199 | 2 | 222008243 | G | A |
| 1471 | EPHA4 | rs3770198 | 2 | 222008301 | A | G |
| 1472 | EPHA4 | rs3770196 | 2 | 222013205 | A | G |
| 1473 | EPHA4 | rs2288628 | 2 | 222015778 | C | A |
| 1474 | EPHA4 | rs16862666 | 2 | 222017088 | T | G |
| 1475 | EPHA4 | rs2052943 | 2 | 222019310 | C | T |
| 1476 | EPHA4 | rs3770181 | 2 | 222022586 | C | T |
| 1477 | EPHA4 | rs3770178 | 2 | 222022726 | C | T |
| 1478 | EPHA4 | rs2052942 | 2 | 222024819 | C | A |
| 1479 | EPHA4 | rs3755036 | 2 | 222025359 | G | A |
| 1480 | EPHA4 | rs3770176 | 2 | 222025789 | G | A |
| 1481 | EPHA4 | rs3815970 | 2 | 222028310 | C | T |
| 1482 | EPHA4 | rs2288627 | 2 | 222028671 | T | C |
| 1483 | EPHA4 | rs16862708 | 2 | 222038366 | G | A |
| 1484 | EPHA4 | rs1430216 | 2 | 222039214 | A | G |
| 1485 | EPHA4 | rs975194 | 2 | 222045870 | T | C |
| 1486 | EPHA4 | rs3770149 | 2 | 222051136 | A | G |
| 1487 | EPHA4 | rs3770146 | 2 | 222052440 | C | T |
| 1488 | EPHA4 | rs1367241 | 2 | 222061657 | G | A |
| 1489 | EPHA4 | rs6436266 | 2 | 222065479 | C | T |
| 1490 | EPHA4 | rs10498114 | 2 | 222067153 | A | G |
| 1491 | EPHA4 | rs7575433 | 2 | 222078114 | G | A |
| 1492 | EPHA4 | rs7558703 | 2 | 222082667 | T | C |
| 1493 | EPHA4 | rs9288570 | 2 | 222087238 | G | A |
| 1494 | EPHA4 | rs2710507 | 2 | 222089044 | G | A |
| 1495 | EPHA4 | rs2710508 | 2 | 222089132 | G | A |
| 1496 | EPHA4 | rs2250204 | 2 | 222108467 | T | C |
| 1497 | EPHA4 | rs1356703 | 2 | 222111843 | C | T |
| 1498 | EPHA4 | rs958165 | 2 | 222113704 | A | G |
| 1499 | EPHA4 | rs2710497 | 2 | 222114812 | C | T |
| 1500 | EPHA4 | rs1568359 | 2 | 222120781 | G | A |
| 1501 | EPHA4 | rs10498118 | 2 | 222131633 | G | A |
| 1502 | EPHA4 | rs17299486 | 2 | 222133409 | A | C |
| 1503 | EPHA4 | rs10498120 | 2 | 222133864 | A | G |
| 1504 | EPHA4 | rs2056290 | 2 | 222136115 | T | C |
| 1505 | EPHA4 | rs3770143 | 2 | 222139227 | A | G |
| 1506 | NGEF | rs709937 | 2 | 233452265 | C | T |
| 1507 | NGEF | rs2292724 | 2 | 233457922 | T | G |
| 1508 | NGEF | rs6719766 | 2 | 233462542 | A | G |
| 1509 | NGEF | rs2166441 | 2 | 233465687 | A | G |
| 1510 | NGEF | rs7355380 | 2 | 233480544 | C | T |
| 1511 | NGEF | rs778356 | 2 | 233492962 | G | A |
| 1512 | NGEF | rs2004203 | 2 | 233498691 | G | A |
| 1513 | NGEF | rs11685503 | 2 | 233501761 | A | G |
| 1514 | NGEF | rs6437075 | 2 | 233507644 | G | A |
| 1515 | NGEF | rs778351 | 2 | 233511635 | C | A |
| 1516 | NGEF | rs4973578 | 2 | 233517488 | C | A |
| 1517 | NGEF | rs6751590 | 2 | 233523122 | C | A |
| 1518 | NGEF | rs6707071 | 2 | 233526575 | G | A |
| 1519 | NGEF | rs2138580 | 2 | 233532360 | C | T |
| 1520 | NGEF | rs2176308 | 2 | 233532447 | G | A |
| 1521 | NGEF | rs11886924 | 2 | 233533154 | C | T |
| 1522 | NGEF | rs11678851 | 2 | 233534191 | C | T |
| 1523 | NGEF | rs2344968 | 2 | 233535031 | A | G |
| 1524 | NGEF | rs13397982 | 2 | 233535510 | A | G |
| 1525 | NGEF | rs11678345 | 2 | 233539081 | C | T |
| 1526 | NGEF | rs11678490 | 2 | 233539194 | G | A |
| 1527 | NGEF | rs4973588 | 2 | 233543219 | G | A |
| 1528 | NGEF | rs10933421 | 2 | 233546272 | T | C |
| 1529 | NGEF | rs3817321 | 2 | 233547296 | C | T |
| 1530 | NGEF | rs6437079 | 2 | 233547418 | A | G |
| 1531 | NGEF | rs6748986 | 2 | 233547483 | C | T |
| 1532 | NGEF | rs6760133 | 2 | 233553749 | T | G |
| 1533 | NGEF | rs11885626 | 2 | 233562641 | A | G |
| 1534 | NGEF | rs6437080 | 2 | 233563705 | G | A |
| 1535 | NGEF | rs4973062 | 2 | 233566880 | T | C |
| 1536 | NGEF | rs10933423 | 2 | 233579711 | A | C |
| 1537 | NGEF | rs10169752 | 2 | 233580293 | G | A |
| 1538 | NGEF | rs10193545 | 2 | 233580344 | C | T |
| 1539 | NGEF | rs10933424 | 2 | 233580652 | T | C |
| 1540 | NGEF | rs13383577 | 2 | 233582643 | T | C |
| 1541 | NGEF | rs3811589 | 2 | 233583960 | G | A |
| 1542 | NGEF | rs3811588 | 2 | 233584219 | C | T |
| 1543 | NGEF | rs6725244 | 2 | 233585309 | T | C |
| 1544 | NGEF | rs6718480 | 2 | 233587310 | A | G |
| 1545 | PAK7 | rs12329540 | 20 | 9468142 | G | A |
| 1546 | PAK7 | rs2072952 | 20 | 9473601 | A | G |
| 1547 | PAK7 | rs6056687 | 20 | 9474228 | T | C |
| 1548 | PAK7 | rs2206386 | 20 | 9478868 | A | G |
| 1549 | PAK7 | rs6039498 | 20 | 9480700 | C | T |
| 1550 | PAK7 | rs6039502 | 20 | 9488838 | G | A |
| 1551 | PAK7 | rs10485733 | 20 | 9492722 | T | C |
| 1552 | PAK7 | rs6133723 | 20 | 9494554 | G | A |
| 1553 | PAK7 | rs6039505 | 20 | 9495288 | T | G |
| 1554 | PAK7 | rs2026339 | 20 | 9502125 | T | C |
| 1555 | PAK7 | rs9967942 | 20 | 9503781 | A | C |
| 1556 | PAK7 | rs6056712 | 20 | 9504908 | C | T |
| 1557 | PAK7 | rs6039512 | 20 | 9510922 | A | G |
| 1558 | PAK7 | rs6077566 | 20 | 9515257 | G | A |
| 1559 | PAK7 | rs6140971 | 20 | 9515361 | G | A |
| 1560 | PAK7 | rs6077567 | 20 | 9517769 | G | A |
| 1561 | PAK7 | rs6056725 | 20 | 9522063 | A | C |
| 1562 | PAK7 | rs6039519 | 20 | 9523688 | A | G |
| 1563 | PAK7 | rs6056732 | 20 | 9524620 | C | A |
| 1564 | PAK7 | rs6056733 | 20 | 9524643 | A | G |
| 1565 | PAK7 | rs6056734 | 20 | 9525379 | C | — |
| 1566 | PAK7 | rs6056740 | 20 | 9529876 | T | C |
| 1567 | PAK7 | rs6140981 | 20 | 9533873 | T | C |
| 1568 | PAK7 | rs928366 | 20 | 9537196 | T | C |
| 1569 | PAK7 | rs2225471 | 20 | 9537577 | T | C |
| 1570 | PAK7 | rs6086959 | 20 | 9538093 | A | C |
| 1571 | PAK7 | rs6056750 | 20 | 9539204 | C | T |
| 1572 | PAK7 | rs6133731 | 20 | 9539628 | C | T |
| 1573 | PAK7 | rs6056755 | 20 | 9540617 | A | G |
| 1574 | PAK7 | rs6039524 | 20 | 9540676 | C | A |
| 1575 | PAK7 | rs7272361 | 20 | 9542881 | T | C |
| 1576 | PAK7 | rs219855 | 20 | 9543352 | A | G |
| 1577 | PAK7 | rs219861 | 20 | 9545555 | A | G |
| 1578 | PAK7 | rs219862 | 20 | 9546144 | G | T |
| 1579 | PAK7 | rs219870 | 20 | 9550875 | T | G |
| 1580 | PAK7 | rs6118672 | 20 | 9551706 | G | A |
| 1581 | PAK7 | rs6039529 | 20 | 9556977 | G | A |
| 1582 | PAK7 | rs6056766 | 20 | 9559448 | G | A |
| 1583 | PAK7 | rs6086964 | 20 | 9559551 | A | C |
| 1584 | PAK7 | rs12480230 | 20 | 9561674 | G | T |
| 1585 | PAK7 | rs16996186 | 20 | 9561817 | G | T |
| 1586 | PAK7 | rs12624866 | 20 | 9564075 | A | G |
| 1587 | PAK7 | rs6056773 | 20 | 9565425 | G | A |
| 1588 | PAK7 | rs6516479 | 20 | 9566761 | A | G |
| 1589 | PAK7 | rs4813894 | 20 | 9567475 | A | G |
| 1590 | PAK7 | rs6056774 | 20 | 9572587 | T | C |
| 1591 | PAK7 | rs10485734 | 20 | 9573787 | G | A |
| 1592 | PAK7 | rs6086972 | 20 | 9575908 | C | A |
| 1593 | PAK7 | rs1885563 | 20 | 9578238 | G | A |
| 1594 | PAK7 | rs6039533 | 20 | 9578908 | G | A |
| 1595 | PAK7 | rs2208756 | 20 | 9579499 | T | — |
| 1596 | PAK7 | rs4816158 | 20 | 9580609 | G | A |
| 1597 | PAK7 | rs6039536 | 20 | 9582410 | C | T |
| 1598 | PAK7 | rs6039540 | 20 | 9589288 | G | A |
| 1599 | PAK7 | rs6118681 | 20 | 9589555 | T | C |
| 1600 | PAK7 | rs17408919 | 20 | 9592206 | C | A |
| 1601 | PAK7 | rs2423395 | 20 | 9593420 | G | A |
| 1602 | PAK7 | rs6133735 | 20 | 9594834 | C | T |
| 1603 | PAK7 | rs6039542 | 20 | 9596401 | C | T |
| 1604 | PAK7 | rs2423401 | 20 | 9597074 | C | T |
| 1605 | PAK7 | rs6118687 | 20 | 9597234 | G | A |
| 1606 | PAK7 | rs6056793 | 20 | 9600776 | T | — |
| 1607 | PAK7 | rs2254000 | 20 | 9603718 | G | A |
| 1608 | PAK7 | rs2254003 | 20 | 9603837 | A | G |
| 1609 | PAK7 | rs2423412 | 20 | 9603984 | G | A |
| 1610 | PAK7 | rs2423413 | 20 | 9606534 | T | C |
| 1611 | PAK7 | rs6039545 | 20 | 9607647 | T | G |
| 1612 | PAK7 | rs6077585 | 20 | 9613895 | T | C |
| 1613 | PAK7 | rs2206476 | 20 | 9618244 | G | A |
| 1614 | PAK7 | rs6141008 | 20 | 9618903 | T | C |
| 1615 | PAK7 | rs2050104 | 20 | 9619084 | T | C |
| 1616 | PAK7 | rs2050105 | 20 | 9620987 | G | A |
| 1617 | PAK7 | rs8120907 | 20 | 9629848 | A | G |
| 1618 | PAK7 | rs2223565 | 20 | 9631180 | T | G |
| 1619 | PAK7 | rs6086987 | 20 | 9633155 | A | G |
| 1620 | PAK7 | rs6056812 | 20 | 9633525 | C | T |
| 1621 | PAK7 | rs6108331 | 20 | 9633613 | C | T |
| 1622 | PAK7 | rs6118709 | 20 | 9647027 | C | T |
| 1623 | PAK7 | rs6086998 | 20 | 9650970 | T | C |
| 1624 | PAK7 | rs2423438 | 20 | 9652943 | T | G |
| 1625 | PAK7 | rs6039561 | 20 | 9657038 | G | A |
| 1626 | PAK7 | rs2423441 | 20 | 9658169 | C | T |
| 1627 | PAK7 | rs2423443 | 20 | 9658347 | A | G |
| 1628 | PAK7 | rs7274475 | 20 | 9661878 | T | G |
| 1629 | PAK7 | rs6516492 | 20 | 9666199 | A | C |
| 1630 | PAK7 | rs2423456 | 20 | 9668742 | A | G |
| 1631 | PAK7 | rs7274775 | 20 | 9671375 | C | T |
| 1632 | PAK7 | rs4813896 | 20 | 9672100 | G | A |
| 1633 | PAK7 | rs1033470 | 20 | 9673697 | A | G |
| 1634 | PAK7 | rs4813897 | 20 | 9674640 | G | A |
| 1635 | PAK7 | rs6141026 | 20 | 9678385 | C | T |
| 1636 | PAK7 | rs13045381 | 20 | 9678921 | C | A |
| 1637 | PAK7 | rs6108334 | 20 | 9679193 | T | C |
| 1638 | PAK7 | rs6133746 | 20 | 9680830 | G | A |
| 1639 | PAK7 | rs2423462 | 20 | 9682770 | T | C |
| 1640 | PAK7 | rs6118717 | 20 | 9685413 | T | C |
| 1641 | PAK7 | rs16996383 | 20 | 9686531 | C | T |
| 1642 | PAK7 | rs13045260 | 20 | 9687367 | C | T |
| 1643 | PAK7 | rs1569625 | 20 | 9688441 | T | C |
| 1644 | PAK7 | rs2423467 | 20 | 9689876 | A | G |
| 1645 | PAK7 | rs10470072 | 20 | 9692737 | C | T |
| 1646 | PAK7 | rs926502 | 20 | 9692973 | T | G |
| 1647 | PAK7 | rs4566434 | 20 | 9693450 | T | C |
| 1648 | PAK7 | rs6118724 | 20 | 9694345 | G | A |
| 1649 | PAK7 | rs2327225 | 20 | 9696128 | A | C |
| 1650 | PAK7 | rs17411374 | 20 | 9696829 | C | T |
| 1651 | PAK7 | rs6141031 | 20 | 9697518 | A | G |
| 1652 | PAK7 | rs6039571 | 20 | 9699806 | G | A |
| 1653 | PAK7 | rs6056855 | 20 | 9700408 | C | A |
| 1654 | PAK7 | rs6108337 | 20 | 9702561 | T | C |
| 1655 | PAK7 | rs17502794 | 20 | 9704655 | G | A |
| 1656 | PAK7 | rs6516498 | 20 | 9707414 | G | A |
| 1657 | PAK7 | rs6039576 | 20 | 9708258 | C | T |
| 1658 | PAK7 | rs2206484 | 20 | 9712069 | C | T |
| 1659 | PAK7 | rs2223569 | 20 | 9712263 | A | C |
| 1660 | PAK7 | rs6056871 | 20 | 9713652 | T | C |
| 1661 | PAK7 | rs6056873 | 20 | 9715834 | G | A |
| 1662 | PAK7 | rs994487 | 20 | 9716982 | C | T |
| 1663 | PAK7 | rs6056875 | 20 | 9719136 | A | G |
| 1664 | PAK7 | rs6141035 | 20 | 9720904 | A | G |
| 1665 | PAK7 | rs10485735 | 20 | 9721877 | G | A |
| 1666 | PAK7 | rs2206481 | 20 | 9723551 | G | T |
| 1667 | PAK7 | rs6141038 | 20 | 9724961 | A | G |
| 1668 | PAK7 | rs6056890 | 20 | 9726530 | G | A |
| 1669 | PAK7 | rs6056891 | 20 | 9726729 | A | G |
| 1670 | PAK7 | rs2050108 | 20 | 9744416 | C | T |
| 1671 | PAK7 | rs6087032 | 20 | 9747063 | C | T |
| 1672 | PAK7 | rs6118746 | 20 | 9749670 | T | C |
| 1673 | PAK7 | rs6516503 | 20 | 9752835 | T | C |
| 1674 | PAK7 | rs6056909 | 20 | 9755527 | T | C |
| 1675 | PAK7 | rs221015 | 20 | 9762706 | T | C |
| 1676 | PAK7 | rs221013 | 20 | 9763528 | C | T |
| 1677 | PAK7 | rs6056922 | 20 | 9765816 | G | A |
| 1678 | PAK7 | rs761226 | 20 | 9769007 | T | C |
| 1679 | NFATC2 | rs4811168 | 20 | 49436682 | T | C |
| 1680 | NFATC2 | rs1052653 | 20 | 49438061 | A | G |
| 1681 | NFATC2 | rs1569736 | 20 | 49439729 | C | A |
| 1682 | NFATC2 | rs6021183 | 20 | 49455556 | T | C |
| 1683 | NFATC2 | rs2426302 | 20 | 49458144 | A | C |
| 1684 | NFATC2 | rs6021193 | 20 | 49470116 | A | G |
| 1685 | NFATC2 | rs761374 | 20 | 49476547 | T | C |
| 1686 | NFATC2 | rs2024581 | 20 | 49477349 | C | T |
| 1687 | NFATC2 | rs6126229 | 20 | 49479044 | T | C |
| 1688 | NFATC2 | rs6013184 | 20 | 49480505 | G | A |
| 1689 | NFATC2 | rs4811174 | 20 | 49481540 | C | T |
| 1690 | NFATC2 | rs6096431 | 20 | 49486048 | T | G |
| 1691 | NFATC2 | rs880324 | 20 | 49487597 | G | A |
| 1692 | NFATC2 | rs228832 | 20 | 49493555 | C | T |
| 1693 | NFATC2 | rs16996025 | 20 | 49494721 | C | T |
| 1694 | NFATC2 | rs228838 | 20 | 49502108 | C | T |
| 1695 | NFATC2 | rs228840 | 20 | 49504665 | G | A |
| 1696 | NFATC2 | rs6091326 | 20 | 49514749 | C | T |
| 1697 | NFATC2 | rs228851 | 20 | 49518712 | G | T |
| 1698 | NFATC2 | rs8119860 | 20 | 49520170 | G | T |
| 1699 | NFATC2 | rs17199748 | 20 | 49521103 | C | T |
| 1700 | NFATC2 | rs6021231 | 20 | 49525694 | T | C |
| 1701 | NFATC2 | rs747063 | 20 | 49535610 | C | T |
| 1702 | NFATC2 | rs11086340 | 20 | 49538271 | C | T |
| 1703 | NFATC2 | rs8123890 | 20 | 49540120 | A | G |
| 1704 | NFATC2 | rs4809847 | 20 | 49541727 | G | T |
| 1705 | NFATC2 | rs6021247 | 20 | 49542387 | A | G |
| 1706 | NFATC2 | rs6123045 | 20 | 49552249 | C | T |
| 1707 | NFATC2 | rs3787189 | 20 | 49557164 | A | G |
| 1708 | NFATC2 | rs4811184 | 20 | 49558078 | T | C |
| 1709 | NFATC2 | rs6067804 | 20 | 49565471 | G | A |
| 1710 | NFATC2 | rs2273642 | 20 | 49566496 | G | A |
| 1711 | NFATC2 | rs6021264 | 20 | 49568090 | A | G |
| 1712 | NFATC2 | rs6067805 | 20 | 49568498 | T | C |
| 1713 | NFATC2 | rs6021266 | 20 | 49572654 | A | G |
| 1714 | NFATC2 | rs6063664 | 20 | 49575231 | T | C |
| 1715 | NFATC2 | rs6067809 | 20 | 49576330 | C | T |
| 1716 | NFATC2 | rs8119787 | 20 | 49580478 | G | A |
| 1717 | NFATC2 | rs6123048 | 20 | 49580807 | A | G |
| 1718 | NFATC2 | rs6021275 | 20 | 49588531 | A | G |
| 1719 | NFATC2 | rs6126251 | 20 | 49597547 | C | T |
| 1720 | MAPK1 | rs2298432 | 22 | 20453189 | C | A |
| 1721 | MAPK1 | rs743409 | 22 | 20459215 | C | T |
| 1722 | MAPK1 | rs2283792 | 22 | 20461125 | G | T |
| 1723 | MAPK1 | rs7290469 | 22 | 20480982 | G | A |
| 1724 | MAPK1 | rs8136902 | 22 | 20495557 | C | T |
| 1725 | MAPK1 | rs1892846 | 22 | 20516479 | C | A |
| 1726 | MAPK1 | rs8141815 | 22 | 20521585 | G | A |
| 1727 | MAPK1 | rs12172554 | 22 | 20525075 | C | T |
| 1728 | MAPK1 | rs9610417 | 22 | 20529970 | C | T |
| 1729 | MAPK1 | rs8136867 | 22 | 20534793 | A | G |
| 1730 | MAPK1 | rs2876981 | 22 | 20535611 | A | C |
| 1731 | LIMK2 | rs5749237 | 22 | 29948958 | A | G |
| 1732 | LIMK2 | rs5997917 | 22 | 29951792 | G | A |
| 1733 | LIMK2 | rs5997919 | 22 | 29953442 | G | A |
| 1734 | LIMK2 | rs5997921 | 22 | 29960896 | G | T |
| 1735 | LIMK2 | rs7287402 | 22 | 29984823 | T | C |
| 1736 | LIMK2 | rs9606828 | 22 | 29993066 | T | C |
| 1737 | LIMK2 | rs2413045 | 22 | 29995862 | G | A |
| 1738 | LIMK2 | rs2040532 | 22 | 29999267 | C | T |
| 1739 | LIMK2 | rs8143159 | 22 | 30001882 | C | T |
| 1740 | LIMK2 | rs4141404 | 22 | 30005185 | G | T |
| 1741 | LIMK2 | rs4615 | 22 | 30007744 | A | G |
| 1742 | LIMK2 | rs1034587 | 22 | 30010240 | C | A |
| 1743 | LIMK2 | rs2239905 | 22 | 30010624 | C | T |
| 1744 | RAC2 | rs933222 | 22 | 35948583 | C | T |
| 1745 | RAC2 | rs739041 | 22 | 35954945 | A | G |
| 1746 | RAC2 | rs2284037 | 22 | 35956048 | A | G |
| 1747 | RAC2 | rs1476002 | 22 | 35960734 | C | T |
| 1748 | RAC2 | rs12170612 | 22 | 35962126 | G | T |
| 1749 | RAC2 | rs5756573 | 22 | 35963721 | G | A |
| 1750 | RAC2 | rs2284038 | 22 | 35965001 | A | G |
| 1751 | RAC2 | rs2239775 | 22 | 35967787 | C | A |
| 1752 | RAC2 | rs2213430 | 22 | 35968906 | C | T |
| 1753 | RAC2 | rs972430 | 22 | 35973510 | G | A |
| 1754 | PLXNB2 | rs2235356 | 22 | 49052476 | A | G |
| 1755 | SRGAP3 | rs2324710 | 3 | 8994748 | C | T |
| 1756 | SRGAP3 | rs341791 | 3 | 8999017 | G | A |
| 1757 | SRGAP3 | rs1043951 | 3 | 8999521 | T | C |
| 1758 | SRGAP3 | rs413789 | 3 | 9009145 | C | A |
| 1759 | SRGAP3 | rs523684 | 3 | 9014469 | G | A |
| 1760 | SRGAP3 | rs486012 | 3 | 9016299 | C | T |
| 1761 | SRGAP3 | rs11715608 | 3 | 9025480 | T | C |
| 1762 | SRGAP3 | rs341788 | 3 | 9025659 | T | C |
| 1763 | SRGAP3 | rs932146 | 3 | 9025800 | G | A |
| 1764 | SRGAP3 | rs1533000 | 3 | 9026580 | T | C |
| 1765 | SRGAP3 | rs383563 | 3 | 9027494 | G | A |
| 1766 | SRGAP3 | rs17798894 | 3 | 9036714 | T | G |
| 1767 | SRGAP3 | rs6443217 | 3 | 9037146 | A | G |
| 1768 | SRGAP3 | rs2662089 | 3 | 9041156 | T | G |
| 1769 | SRGAP3 | rs370477 | 3 | 9042597 | T | C |
| 1770 | SRGAP3 | rs1703127 | 3 | 9043858 | T | C |
| 1771 | SRGAP3 | rs370526 | 3 | 9045495 | G | A |
| 1772 | SRGAP3 | rs1513315 | 3 | 9047286 | A | G |
| 1773 | SRGAP3 | rs6803548 | 3 | 9047574 | T | C |
| 1774 | SRGAP3 | rs7617156 | 3 | 9047878 | C | T |
| 1775 | SRGAP3 | rs6762129 | 3 | 9049865 | C | T |
| 1776 | SRGAP3 | rs3755784 | 3 | 9051137 | C | A |
| 1777 | SRGAP3 | rs9875468 | 3 | 9052896 | A | G |
| 1778 | SRGAP3 | rs557377 | 3 | 9053120 | G | A |
| 1779 | SRGAP3 | rs2670009 | 3 | 9053201 | T | G |
| 1780 | SRGAP3 | rs2662088 | 3 | 9053581 | G | A |
| 1781 | SRGAP3 | rs17049971 | 3 | 9054291 | G | A |
| 1782 | SRGAP3 | rs884237 | 3 | 9054896 | T | C |
| 1783 | SRGAP3 | rs639057 | 3 | 9055893 | G | T |
| 1784 | SRGAP3 | rs1183604 | 3 | 9056319 | A | G |
| 1785 | SRGAP3 | rs1162326 | 3 | 9056339 | T | G |
| 1786 | SRGAP3 | rs2670008 | 3 | 9057851 | T | C |
| 1787 | SRGAP3 | rs667714 | 3 | 9060123 | C | T |
| 1788 | SRGAP3 | rs2670003 | 3 | 9062967 | G | A |
| 1789 | SRGAP3 | rs2670002 | 3 | 9065673 | G | T |
| 1790 | SRGAP3 | rs611406 | 3 | 9065905 | G | A |
| 1791 | SRGAP3 | rs2662090 | 3 | 9066946 | C | A |
| 1792 | SRGAP3 | rs7609881 | 3 | 9067661 | G | A |
| 1793 | SRGAP3 | rs13091604 | 3 | 9069531 | T | C |
| 1794 | SRGAP3 | rs2664098 | 3 | 9073423 | T | C |
| 1795 | SRGAP3 | rs7644129 | 3 | 9076597 | C | A |
| 1796 | SRGAP3 | rs6443220 | 3 | 9081968 | T | C |
| 1797 | SRGAP3 | rs7632256 | 3 | 9082475 | T | C |
| 1798 | SRGAP3 | rs1162395 | 3 | 9086221 | C | T |
| 1799 | SRGAP3 | rs13088083 | 3 | 9086847 | T | G |
| 1800 | SRGAP3 | rs1543144 | 3 | 9088024 | G | A |
| 1801 | SRGAP3 | rs2675174 | 3 | 9088057 | G | A |
| 1802 | SRGAP3 | rs13094661 | 3 | 9088278 | A | G |
| 1803 | SRGAP3 | rs2675173 | 3 | 9089327 | G | A |
| 1804 | SRGAP3 | rs6793348 | 3 | 9089759 | T | C |
| 1805 | SRGAP3 | rs9825026 | 3 | 9092788 | A | G |
| 1806 | SRGAP3 | rs1996299 | 3 | 9094182 | C | T |
| 1807 | SRGAP3 | rs2669989 | 3 | 9095004 | G | A |
| 1808 | SRGAP3 | rs2664084 | 3 | 9102694 | G | A |
| 1809 | SRGAP3 | rs2664079 | 3 | 9106599 | C | A |
| 1810 | SRGAP3 | rs2664076 | 3 | 9108971 | C | T |
| 1811 | SRGAP3 | rs531719 | 3 | 9111060 | T | C |
| 1812 | SRGAP3 | rs1861941 | 3 | 9122178 | A | G |
| 1813 | SRGAP3 | rs372576 | 3 | 9123183 | T | C |
| 1814 | SRGAP3 | rs371214 | 3 | 9126362 | T | C |
| 1815 | SRGAP3 | rs369068 | 3 | 9128130 | C | T |
| 1816 | SRGAP3 | rs391923 | 3 | 9131033 | A | G |
| 1817 | SRGAP3 | rs382530 | 3 | 9131817 | G | A |
| 1818 | SRGAP3 | rs17050039 | 3 | 9132737 | A | C |
| 1819 | SRGAP3 | rs422552 | 3 | 9133138 | T | C |
| 1820 | SRGAP3 | rs17050048 | 3 | 9135213 | G | A |
| 1821 | SRGAP3 | rs2543491 | 3 | 9135465 | A | G |
| 1822 | SRGAP3 | rs2664122 | 3 | 9136375 | C | T |
| 1823 | SRGAP3 | rs6781236 | 3 | 9138206 | G | A |
| 1824 | SRGAP3 | rs9853191 | 3 | 9145534 | G | A |
| 1825 | SRGAP3 | rs1300250 | 3 | 9147945 | C | A |
| 1826 | SRGAP3 | rs2675188 | 3 | 9151074 | C | T |
| 1827 | SRGAP3 | rs2675187 | 3 | 9154971 | T | G |
| 1828 | SRGAP3 | rs2668361 | 3 | 9156403 | G | A |
| 1829 | SRGAP3 | rs9827505 | 3 | 9162812 | T | C |
| 1830 | SRGAP3 | rs2257056 | 3 | 9163615 | T | G |
| 1831 | SRGAP3 | rs2675186 | 3 | 9168324 | G | A |
| 1832 | SRGAP3 | rs6762693 | 3 | 9170331 | T | C |
| 1833 | SRGAP3 | rs2675181 | 3 | 9172231 | A | G |
| 1834 | SRGAP3 | rs17530219 | 3 | 9173120 | T | C |
| 1835 | SRGAP3 | rs17530358 | 3 | 9175375 | G | A |
| 1836 | SRGAP3 | rs2664108 | 3 | 9182125 | C | T |
| 1837 | SRGAP3 | rs986119 | 3 | 9186356 | A | C |
| 1838 | SRGAP3 | rs396323 | 3 | 9190296 | C | T |
| 1839 | SRGAP3 | rs400052 | 3 | 9193727 | C | T |
| 1840 | SRGAP3 | rs452714 | 3 | 9194440 | T | G |
| 1841 | SRGAP3 | rs730901 | 3 | 9196399 | C | T |
| 1842 | SRGAP3 | rs802792 | 3 | 9199175 | G | A |
| 1843 | SRGAP3 | rs418207 | 3 | 9200376 | G | A |
| 1844 | SRGAP3 | rs1154386 | 3 | 9204433 | T | C |
| 1845 | SRGAP3 | rs10490820 | 3 | 9205006 | G | A |
| 1846 | SRGAP3 | rs1145153 | 3 | 9209763 | G | A |
| 1847 | SRGAP3 | rs6776837 | 3 | 9210957 | A | G |
| 1848 | SRGAP3 | rs6781447 | 3 | 9215887 | C | T |
| 1849 | SRGAP3 | rs1145157 | 3 | 9216749 | C | T |
| 1850 | SRGAP3 | rs1145158 | 3 | 9220529 | C | T |
| 1851 | SRGAP3 | rs1642756 | 3 | 9223379 | A | G |
| 1852 | SRGAP3 | rs6797462 | 3 | 9225653 | C | T |
| 1853 | SRGAP3 | rs441300 | 3 | 9225949 | T | C |
| 1854 | SRGAP3 | rs9819189 | 3 | 9226198 | T | C |
| 1855 | SRGAP3 | rs12638703 | 3 | 9227998 | C | T |
| 1856 | SRGAP3 | rs1154392 | 3 | 9228191 | G | A |
| 1857 | SRGAP3 | rs390151 | 3 | 9230418 | G | A |
| 1858 | SRGAP3 | rs4684627 | 3 | 9231398 | T | C |
| 1859 | SRGAP3 | rs1710903 | 3 | 9232980 | A | G |
| 1860 | SRGAP3 | rs1154398 | 3 | 9233218 | T | C |
| 1861 | SRGAP3 | rs1995994 | 3 | 9237650 | G | A |
| 1862 | SRGAP3 | rs1400157 | 3 | 9239152 | T | C |
| 1863 | SRGAP3 | rs1028154 | 3 | 9240620 | G | A |
| 1864 | SRGAP3 | rs2700331 | 3 | 9241056 | G | A |
| 1865 | SRGAP3 | rs1154399 | 3 | 9243507 | C | T |
| 1866 | SRGAP3 | rs756235 | 3 | 9243931 | A | G |
| 1867 | SRGAP3 | rs2700326 | 3 | 9246859 | A | G |
| 1868 | SRGAP3 | rs1162707 | 3 | 9247213 | A | G |
| 1869 | SRGAP3 | rs2700325 | 3 | 9248116 | T | G |
| 1870 | SRGAP3 | rs9853725 | 3 | 9248196 | T | C |
| 1871 | SRGAP3 | rs1162704 | 3 | 9249895 | C | T |
| 1872 | SRGAP3 | rs938116 | 3 | 9251083 | A | C |
| 1873 | SRGAP3 | rs2875427 | 3 | 9258299 | T | C |
| 1874 | SRGAP3 | rs9825800 | 3 | 9262737 | C | T |
| 1875 | SRGAP3 | rs2137216 | 3 | 9263545 | T | C |
| 1876 | SRGAP3 | rs2600174 | 3 | 9264086 | C | A |
| 1877 | SRGAP3 | rs11131163 | 3 | 9266861 | A | G |
| 1878 | SRGAP3 | rs2648521 | 3 | 9267368 | G | A |
| 1879 | SRGAP3 | rs1512053 | 3 | 9268722 | G | A |
| 1880 | SRGAP3 | rs2600175 | 3 | 9269880 | A | G |
| 1881 | RHOA | rs3448 | 3 | 49371755 | C | T |
| 1882 | RHOA | rs11716445 | 3 | 49381099 | G | A |
| 1883 | SEMA3F | rs2624842 | 3 | 50171422 | T | G |
| 1884 | SEMA3F | rs2624839 | 3 | 50177235 | A | G |
| 1885 | GNAI2 | rs11716295 | 3 | 50245169 | A | C |
| 1886 | GNAI2 | rs2282751 | 3 | 50266789 | G | A |
| 1887 | GNAI2 | rs2236944 | 3 | 50267197 | G | T |
| 1888 | ROBO2 | rs11127575 | 3 | 77169534 | T | G |
| 1889 | ROBO2 | rs3923745 | 3 | 77172085 | A | G |
| 1890 | ROBO2 | rs6784307 | 3 | 77176729 | C | A |
| 1891 | ROBO2 | rs3934881 | 3 | 77177489 | T | C |
| 1892 | ROBO2 | rs6779154 | 3 | 77187229 | T | C |
| 1893 | ROBO2 | rs12714456 | 3 | 77187286 | G | T |
| 1894 | ROBO2 | rs4377518 | 3 | 77193012 | C | A |
| 1895 | ROBO2 | rs7640155 | 3 | 77197618 | C | T |
| 1896 | ROBO2 | rs4515097 | 3 | 77204396 | C | T |
| 1897 | ROBO2 | rs4459934 | 3 | 77206651 | A | G |
| 1898 | ROBO2 | rs7628843 | 3 | 77216563 | A | G |
| 1899 | ROBO2 | rs9816491 | 3 | 77220968 | A | G |
| 1900 | ROBO2 | rs9873691 | 3 | 77229729 | C | A |
| 1901 | ROBO2 | rs6788280 | 3 | 77229887 | C | — |
| 1902 | ROBO2 | rs4624600 | 3 | 77234477 | A | G |
| 1903 | ROBO2 | rs9859970 | 3 | 77237123 | T | C |
| 1904 | ROBO2 | rs13322517 | 3 | 77238514 | A | G |
| 1905 | ROBO2 | rs13061481 | 3 | 77240877 | T | C |
| 1906 | ROBO2 | rs12185957 | 3 | 77247986 | A | C |
| 1907 | ROBO2 | rs6796664 | 3 | 77250165 | A | G |
| 1908 | ROBO2 | rs6769992 | 3 | 77250858 | G | T |
| 1909 | ROBO2 | rs6785747 | 3 | 77252661 | A | G |
| 1910 | ROBO2 | rs4077419 | 3 | 77270731 | A | C |
| 1911 | ROBO2 | rs7612655 | 3 | 77278220 | C | T |
| 1912 | ROBO2 | rs7615545 | 3 | 77278861 | G | A |
| 1913 | ROBO2 | rs12492882 | 3 | 77280329 | G | A |
| 1914 | ROBO2 | rs7432300 | 3 | 77289777 | T | G |
| 1915 | ROBO2 | rs7433251 | 3 | 77292852 | A | G |
| 1916 | ROBO2 | rs7428284 | 3 | 77301698 | G | T |
| 1917 | ROBO2 | rs6771293 | 3 | 77320339 | C | A |
| 1918 | ROBO2 | rs9852548 | 3 | 77320760 | G | A |
| 1919 | ROBO2 | rs4683981 | 3 | 77322791 | A | G |
| 1920 | ROBO2 | rs11709672 | 3 | 77326175 | T | C |
| 1921 | ROBO2 | rs9822245 | 3 | 77330940 | T | G |
| 1922 | ROBO2 | rs1110226 | 3 | 77332387 | A | G |
| 1923 | ROBO2 | rs9812479 | 3 | 77332427 | G | A |
| 1924 | ROBO2 | rs17015082 | 3 | 77333982 | A | G |
| 1925 | ROBO2 | rs11713960 | 3 | 77335882 | G | T |
| 1926 | ROBO2 | rs4684028 | 3 | 77355345 | A | G |
| 1927 | ROBO2 | rs4684030 | 3 | 77356292 | A | G |
| 1928 | ROBO2 | rs7432274 | 3 | 77356601 | A | G |
| 1929 | ROBO2 | rs12487684 | 3 | 77364367 | A | G |
| 1930 | ROBO2 | rs17769969 | 3 | 77366337 | G | A |
| 1931 | ROBO2 | rs6772736 | 3 | 77368734 | G | A |
| 1932 | ROBO2 | rs6768395 | 3 | 77370795 | T | C |
| 1933 | ROBO2 | rs975808 | 3 | 77373535 | A | G |
| 1934 | ROBO2 | rs12633766 | 3 | 77373740 | G | A |
| 1935 | ROBO2 | rs6764328 | 3 | 77378351 | C | T |
| 1936 | ROBO2 | rs4072806 | 3 | 77382124 | C | A |
| 1937 | ROBO2 | rs7430171 | 3 | 77391013 | C | T |
| 1938 | ROBO2 | rs1372423 | 3 | 77393054 | A | G |
| 1939 | ROBO2 | rs1470203 | 3 | 77398108 | G | A |
| 1940 | ROBO2 | rs17015112 | 3 | 77402177 | A | G |
| 1941 | ROBO2 | rs13064976 | 3 | 77403172 | G | T |
| 1942 | ROBO2 | rs17822079 | 3 | 77403430 | C | T |
| 1943 | ROBO2 | rs9882239 | 3 | 77405024 | T | C |
| 1944 | ROBO2 | rs6776288 | 3 | 77405970 | T | C |
| 1945 | ROBO2 | rs11718389 | 3 | 77408107 | T | C |
| 1946 | ROBO2 | rs9309764 | 3 | 77408431 | G | A |
| 1947 | ROBO2 | rs1992449 | 3 | 77411694 | A | G |
| 1948 | ROBO2 | rs10511057 | 3 | 77412082 | T | C |
| 1949 | ROBO2 | rs13087163 | 3 | 77412228 | C | A |
| 1950 | ROBO2 | rs1441956 | 3 | 77415340 | G | A |
| 1951 | ROBO2 | rs7433256 | 3 | 77416455 | G | A |
| 1952 | ROBO2 | rs2875436 | 3 | 77418846 | G | A |
| 1953 | ROBO2 | rs9848676 | 3 | 77420445 | A | G |
| 1954 | ROBO2 | rs998037 | 3 | 77429653 | C | T |
| 1955 | ROBO2 | rs1899373 | 3 | 77436100 | G | A |
| 1956 | ROBO2 | rs11127586 | 3 | 77439977 | C | T |
| 1957 | ROBO2 | rs10511054 | 3 | 77442049 | C | T |
| 1958 | ROBO2 | rs1372427 | 3 | 77451048 | A | G |
| 1959 | ROBO2 | rs1978940 | 3 | 77452601 | C | A |
| 1960 | ROBO2 | rs2028510 | 3 | 77460223 | G | T |
| 1961 | ROBO2 | rs2028512 | 3 | 77466146 | T | C |
| 1962 | ROBO2 | rs9868442 | 3 | 77466640 | G | A |
| 1963 | ROBO2 | rs10511055 | 3 | 77469825 | A | G |
| 1964 | ROBO2 | rs6548499 | 3 | 77472056 | C | T |
| 1965 | ROBO2 | rs4449296 | 3 | 77477230 | A | G |
| 1966 | ROBO2 | rs4539916 | 3 | 77479334 | G | A |
| 1967 | ROBO2 | rs10865559 | 3 | 77489198 | T | C |
| 1968 | ROBO2 | rs4462943 | 3 | 77490741 | C | T |
| 1969 | ROBO2 | rs4578998 | 3 | 77490833 | G | A |
| 1970 | ROBO2 | rs7642312 | 3 | 77491880 | G | A |
| 1971 | ROBO2 | rs9869100 | 3 | 77494923 | A | G |
| 1972 | ROBO2 | rs12493232 | 3 | 77495032 | T | C |
| 1973 | ROBO2 | rs6767250 | 3 | 77497441 | G | T |
| 1974 | ROBO2 | rs9809640 | 3 | 77497752 | A | G |
| 1975 | ROBO2 | rs9876951 | 3 | 77501113 | A | G |
| 1976 | ROBO2 | rs4683973 | 3 | 77501549 | C | T |
| 1977 | ROBO2 | rs9872037 | 3 | 77507390 | A | G |
| 1978 | ROBO2 | rs9872529 | 3 | 77507837 | C | T |
| 1979 | ROBO2 | rs12493938 | 3 | 77510177 | G | A |
| 1980 | ROBO2 | rs4452305 | 3 | 77510261 | A | G |
| 1981 | ROBO2 | rs6768736 | 3 | 77513320 | G | A |
| 1982 | ROBO2 | rs6548505 | 3 | 77516662 | A | G |
| 1983 | ROBO2 | rs9873887 | 3 | 77518313 | C | T |
| 1984 | ROBO2 | rs4684005 | 3 | 77520493 | T | G |
| 1985 | ROBO2 | rs9836971 | 3 | 77525971 | G | A |
| 1986 | ROBO2 | rs6548507 | 3 | 77527056 | G | A |
| 1987 | ROBO2 | rs9883718 | 3 | 77527122 | A | G |
| 1988 | ROBO2 | rs11127590 | 3 | 77532558 | C | T |
| 1989 | ROBO2 | rs7432690 | 3 | 77533053 | C | T |
| 1990 | ROBO2 | rs12492221 | 3 | 77538615 | G | A |
| 1991 | ROBO2 | rs4327364 | 3 | 77544045 | G | T |
| 1992 | ROBO2 | rs9654017 | 3 | 77551573 | G | A |
| 1993 | ROBO2 | rs9830337 | 3 | 77554152 | A | G |
| 1994 | ROBO2 | rs4459877 | 3 | 77556151 | G | A |
| 1995 | ROBO2 | rs9861539 | 3 | 77556683 | T | G |
| 1996 | ROBO2 | rs11127593 | 3 | 77557508 | C | T |
| 1997 | ROBO2 | rs11127595 | 3 | 77557821 | G | A |
| 1998 | ROBO2 | rs9817088 | 3 | 77564025 | A | G |
| 1999 | ROBO2 | rs12634690 | 3 | 77568828 | C | T |
| 2000 | ROBO2 | rs6777631 | 3 | 77581314 | C | A |
| 2001 | ROBO2 | rs6793923 | 3 | 77582062 | T | C |
| 2002 | ROBO2 | rs9840819 | 3 | 77586188 | G | A |
| 2003 | ROBO2 | rs11127596 | 3 | 77586264 | C | T |
| 2004 | ROBO2 | rs9872416 | 3 | 77588395 | C | T |
| 2005 | ROBO2 | rs7651943 | 3 | 77590214 | C | T |
| 2006 | ROBO2 | rs2166802 | 3 | 77592144 | G | T |
| 2007 | ROBO2 | rs4684011 | 3 | 77600301 | C | T |
| 2008 | ROBO2 | rs7614439 | 3 | 77603996 | T | C |
| 2009 | ROBO2 | rs1345179 | 3 | 77607823 | C | T |
| 2010 | ROBO2 | rs12634489 | 3 | 77608755 | G | A |
| 2011 | ROBO2 | rs12487172 | 3 | 77610217 | G | A |
| 2012 | ROBO2 | rs7610881 | 3 | 77619036 | A | G |
| 2013 | ROBO2 | rs13064369 | 3 | 77621908 | T | C |
| 2014 | ROBO2 | rs7432637 | 3 | 77624724 | C | T |
| 2015 | ROBO2 | rs17015300 | 3 | 77626511 | T | C |
| 2016 | ROBO2 | rs6797725 | 3 | 77627512 | C | T |
| 2017 | ROBO2 | rs6779560 | 3 | 77628954 | A | G |
| 2018 | ROBO2 | rs13077150 | 3 | 77641075 | G | A |
| 2019 | ROBO2 | rs935526 | 3 | 77642707 | T | C |
| 2020 | ROBO2 | rs1447846 | 3 | 77652511 | G | A |
| 2021 | ROBO2 | rs796529 | 3 | 77662905 | G | T |
| 2022 | ROBO2 | rs6771083 | 3 | 77665561 | T | C |
| 2023 | ROBO2 | rs775760 | 3 | 77669287 | C | A |
| 2024 | ROBO2 | rs775762 | 3 | 77671179 | T | C |
| 2025 | ROBO2 | rs775764 | 3 | 77672607 | C | T |
| 2026 | ROBO2 | rs1666130 | 3 | 77681633 | A | G |
| 2027 | ROBO2 | rs1523759 | 3 | 77686932 | T | C |
| 2028 | ROBO2 | rs10514734 | 3 | 77687380 | C | T |
| 2029 | ROBO2 | rs1403848 | 3 | 77692345 | A | C |
| 2030 | ROBO2 | rs808966 | 3 | 77692466 | G | A |
| 2031 | ROBO2 | rs796527 | 3 | 77702083 | A | C |
| 2032 | ROBO2 | rs1107432 | 3 | 77715084 | C | T |
| 2033 | ROBO2 | rs775733 | 3 | 77720357 | C | T |
| 2034 | ROBO2 | rs1721175 | 3 | 77733384 | T | C |
| 2035 | ROBO2 | rs9852724 | 3 | 77734675 | C | T |
| 2036 | ROBO2 | rs1523768 | 3 | 77749734 | T | C |
| 2037 | ROBO2 | rs9848106 | 3 | 77751036 | A | G |
| 2038 | ROBO2 | rs4684016 | 3 | 77756598 | A | G |
| 2039 | ROBO2 | rs876675 | 3 | 77758328 | A | G |
| 2040 | ROBO2 | rs10779982 | 3 | 77760996 | G | A |
| 2041 | ROBO2 | rs3821735 | 3 | 77766912 | G | A |
| 2042 | ROBO2 | rs1839795 | 3 | 77775815 | C | T |
| 2043 | ROBO2 | rs1031377 | 3 | 77779112 | T | C |
| 2044 | ROBO2 | rs10865561 | 3 | 77781819 | C | T |
| 2045 | ROBO2 | rs776136 | 3 | 77782724 | T | C |
| 2046 | ROBO2 | rs809603 | 3 | 77783731 | T | C |
| 2047 | ROBO1 | rs11706918 | 3 | 78725740 | A | G |
| 2048 | ROBO1 | rs6786778 | 3 | 78733250 | A | G |
| 2049 | ROBO1 | rs723766 | 3 | 78740464 | G | A |
| 2050 | ROBO1 | rs723765 | 3 | 78740688 | G | A |
| 2051 | ROBO1 | rs1027828 | 3 | 78743375 | C | T |
| 2052 | ROBO1 | rs3773195 | 3 | 78745964 | G | A |
| 2053 | ROBO1 | rs2872006 | 3 | 78746324 | A | G |
| 2054 | ROBO1 | rs1027832 | 3 | 78746646 | G | A |
| 2055 | ROBO1 | rs1025946 | 3 | 78747342 | T | C |
| 2056 | ROBO1 | rs3773201 | 3 | 78751143 | C | T |
| 2057 | ROBO1 | rs17016413 | 3 | 78753344 | C | T |
| 2058 | ROBO1 | rs3773207 | 3 | 78758806 | C | T |
| 2059 | ROBO1 | rs7625555 | 3 | 78759095 | C | T |
| 2060 | ROBO1 | rs6809232 | 3 | 78774966 | A | G |
| 2061 | ROBO1 | rs2660739 | 3 | 78776530 | G | A |
| 2062 | ROBO1 | rs1032966 | 3 | 78781078 | G | A |
| 2063 | ROBO1 | rs998146 | 3 | 78787332 | G | A |
| 2064 | ROBO1 | rs7613490 | 3 | 78794474 | G | A |
| 2065 | ROBO1 | rs7613523 | 3 | 78794672 | C | — |
| 2066 | ROBO1 | rs2043661 | 3 | 78816411 | A | G |
| 2067 | ROBO1 | rs767182 | 3 | 78837321 | C | T |
| 2068 | ROBO1 | rs984138 | 3 | 78839493 | G | A |
| 2069 | ROBO1 | rs6787349 | 3 | 78851632 | C | T |
| 2070 | ROBO1 | rs7626082 | 3 | 78856724 | A | C |
| 2071 | ROBO1 | rs3773232 | 3 | 78856945 | C | T |
| 2072 | ROBO1 | rs3773234 | 3 | 78860201 | A | G |
| 2073 | ROBO1 | rs11927671 | 3 | 78870559 | G | T |
| 2074 | ROBO1 | rs2608021 | 3 | 78872052 | G | A |
| 2075 | ROBO1 | rs17016495 | 3 | 78875814 | C | T |
| 2076 | ROBO1 | rs2304503 | 3 | 78878768 | A | C |
| 2077 | ROBO1 | rs1865862 | 3 | 78886540 | C | A |
| 2078 | ROBO1 | rs510006 | 3 | 78886625 | G | A |
| 2079 | ROBO1 | rs1865861 | 3 | 78886649 | A | G |
| 2080 | ROBO1 | rs11716325 | 3 | 78890705 | A | G |
| 2081 | ROBO1 | rs333491 | 3 | 78891540 | T | C |
| 2082 | ROBO1 | rs333472 | 3 | 78907602 | T | C |
| 2083 | ROBO1 | rs383189 | 3 | 78913331 | G | — |
| 2084 | ROBO1 | rs333507 | 3 | 78923643 | T | C |
| 2085 | ROBO1 | rs7650002 | 3 | 78926964 | G | A |
| 2086 | ROBO1 | rs9309819 | 3 | 78930722 | C | T |
| 2087 | ROBO1 | rs11127635 | 3 | 78940492 | A | G |
| 2088 | ROBO1 | rs1355983 | 3 | 78945368 | T | G |
| 2089 | ROBO1 | rs7629522 | 3 | 78945848 | C | T |
| 2090 | ROBO1 | rs162870 | 3 | 78959907 | A | C |
| 2091 | ROBO1 | rs162871 | 3 | 78960199 | C | A |
| 2092 | ROBO1 | rs7617433 | 3 | 78966169 | G | A |
| 2093 | ROBO1 | rs1843786 | 3 | 78967414 | A | G |
| 2094 | ROBO1 | rs331158 | 3 | 78975042 | C | A |
| 2095 | ROBO1 | rs17377726 | 3 | 78978925 | G | A |
| 2096 | ROBO1 | rs9835958 | 3 | 78979288 | T | C |
| 2097 | ROBO1 | rs1480689 | 3 | 78983702 | C | A |
| 2098 | ROBO1 | rs329810 | 3 | 78984976 | T | C |
| 2099 | ROBO1 | rs1383407 | 3 | 78990861 | T | C |
| 2100 | ROBO1 | rs1031932 | 3 | 78995893 | A | C |
| 2101 | ROBO1 | rs331149 | 3 | 78997895 | A | G |
| 2102 | ROBO1 | rs331146 | 3 | 78999973 | A | G |
| 2103 | ROBO1 | rs11127636 | 3 | 79005542 | A | C |
| 2104 | ROBO1 | rs331199 | 3 | 79033572 | T | C |
| 2105 | ROBO1 | rs329814 | 3 | 79039799 | T | G |
| 2106 | ROBO1 | rs498805 | 3 | 79051531 | T | C |
| 2107 | ROBO1 | rs328047 | 3 | 79054730 | T | C |
| 2108 | ROBO1 | rs9872429 | 3 | 79058672 | A | G |
| 2109 | ROBO1 | rs17313129 | 3 | 79060197 | C | T |
| 2110 | ROBO1 | rs9839572 | 3 | 79066613 | A | C |
| 2111 | ROBO1 | rs328049 | 3 | 79068495 | C | T |
| 2112 | ROBO1 | rs17313732 | 3 | 79096568 | G | A |
| 2113 | ROBO1 | rs9832632 | 3 | 79098154 | C | T |
| 2114 | ROBO1 | rs17016656 | 3 | 79103329 | A | G |
| 2115 | ROBO1 | rs1009682 | 3 | 79104086 | G | A |
| 2116 | ROBO1 | rs17379690 | 3 | 79107231 | T | G |
| 2117 | ROBO1 | rs4680919 | 3 | 79108259 | T | C |
| 2118 | ROBO1 | rs1447833 | 3 | 79122096 | A | G |
| 2119 | ROBO1 | rs10084694 | 3 | 79129159 | C | — |
| 2120 | ROBO1 | rs6766522 | 3 | 79160577 | G | A |
| 2121 | ROBO1 | rs6548612 | 3 | 79206160 | C | T |
| 2122 | ROBO1 | rs4680953 | 3 | 79209700 | C | — |
| 2123 | ROBO1 | rs1455824 | 3 | 79219920 | G | A |
| 2124 | ROBO1 | rs9866364 | 3 | 79240044 | T | C |
| 2125 | ROBO1 | rs17315187 | 3 | 79252565 | T | C |
| 2126 | ROBO1 | rs6786179 | 3 | 79259720 | G | A |
| 2127 | ROBO1 | rs7349498 | 3 | 79268774 | G | A |
| 2128 | ROBO1 | rs10865573 | 3 | 79278187 | C | T |
| 2129 | ROBO1 | rs12486635 | 3 | 79280400 | A | G |
| 2130 | ROBO1 | rs9823652 | 3 | 79304261 | G | A |
| 2131 | ROBO1 | rs4334606 | 3 | 79312209 | C | T |
| 2132 | ROBO1 | rs2077753 | 3 | 79347301 | C | T |
| 2133 | ROBO1 | rs13087498 | 3 | 79357275 | T | C |
| 2134 | ROBO1 | rs324755 | 3 | 79358278 | A | G |
| 2135 | ROBO1 | rs11924816 | 3 | 79369491 | C | T |
| 2136 | ROBO1 | rs9880736 | 3 | 79401678 | A | C |
| 2137 | ROBO1 | rs1489850 | 3 | 79411849 | T | C |
| 2138 | ROBO1 | rs11925648 | 3 | 79419239 | T | C |
| 2139 | ROBO1 | rs7618349 | 3 | 79422280 | T | C |
| 2140 | ROBO1 | rs9832396 | 3 | 79436877 | G | A |
| 2141 | ROBO1 | rs6769328 | 3 | 79455526 | A | G |
| 2142 | ROBO1 | rs4362686 | 3 | 79476047 | G | A |
| 2143 | ROBO1 | rs13067596 | 3 | 79481175 | C | T |
| 2144 | ROBO1 | rs13073607 | 3 | 79482170 | C | T |
| 2145 | ROBO1 | rs6548616 | 3 | 79482265 | T | C |
| 2146 | ROBO1 | rs7622139 | 3 | 79487383 | C | T |
| 2147 | ROBO1 | rs1489848 | 3 | 79487728 | G | T |
| 2148 | ROBO1 | rs9842630 | 3 | 79489209 | C | T |
| 2149 | ROBO1 | rs6802848 | 3 | 79493704 | G | A |
| 2150 | ROBO1 | rs12633213 | 3 | 79504372 | T | C |
| 2151 | ROBO1 | rs1387665 | 3 | 79512501 | G | A |
| 2152 | ROBO1 | rs7611676 | 3 | 79524043 | C | T |
| 2153 | ROBO1 | rs4680960 | 3 | 79532256 | C | T |
| 2154 | ROBO1 | rs4130991 | 3 | 79551712 | A | G |
| 2155 | ROBO1 | rs7653197 | 3 | 79553474 | C | A |
| 2156 | ROBO1 | rs4535189 | 3 | 79572661 | T | C |
| 2157 | ROBO1 | rs6803202 | 3 | 79581843 | C | T |
| 2158 | ROBO1 | rs7628757 | 3 | 79605062 | A | G |
| 2159 | ROBO1 | rs12635896 | 3 | 79613438 | T | C |
| 2160 | ROBO1 | rs4564923 | 3 | 79615785 | C | T |
| 2161 | ROBO1 | rs7612183 | 3 | 79619602 | T | C |
| 2162 | ROBO1 | rs4264688 | 3 | 79629038 | G | A |
| 2163 | ROBO1 | rs6548621 | 3 | 79633063 | G | A |
| 2164 | ROBO1 | rs7622444 | 3 | 79640617 | A | G |
| 2165 | ROBO1 | rs7637338 | 3 | 79643294 | G | A |
| 2166 | ROBO1 | rs7614913 | 3 | 79650319 | T | C |
| 2167 | ROBO1 | rs6548628 | 3 | 79652248 | C | A |
| 2168 | ROBO1 | rs9853895 | 3 | 79667848 | C | T |
| 2169 | ROBO1 | rs9857859 | 3 | 79673997 | C | T |
| 2170 | ROBO1 | rs7623728 | 3 | 79679725 | C | T |
| 2171 | ROBO1 | rs7617400 | 3 | 79686754 | C | T |
| 2172 | ROBO1 | rs11924965 | 3 | 79693315 | C | T |
| 2173 | ROBO1 | rs9863111 | 3 | 79711784 | A | G |
| 2174 | ROBO1 | rs12714483 | 3 | 79720108 | T | G |
| 2175 | ROBO1 | rs7432676 | 3 | 79721054 | G | T |
| 2176 | ROBO1 | rs9855098 | 3 | 79722265 | C | A |
| 2177 | EPHA3 | rs7642606 | 3 | 89251573 | G | T |
| 2178 | EPHA3 | rs7429534 | 3 | 89273999 | C | T |
| 2179 | EPHA3 | rs10779990 | 3 | 89291271 | C | T |
| 2180 | EPHA3 | rs12633482 | 3 | 89295631 | G | A |
| 2181 | EPHA3 | rs9815025 | 3 | 89297903 | G | A |
| 2182 | EPHA3 | rs7433757 | 3 | 89300019 | G | A |
| 2183 | EPHA3 | rs12486049 | 3 | 89323970 | T | G |
| 2184 | EPHA3 | rs4132275 | 3 | 89330571 | A | G |
| 2185 | EPHA3 | rs7373232 | 3 | 89350956 | A | G |
| 2186 | EPHA3 | rs6551407 | 3 | 89353295 | T | C |
| 2187 | EPHA3 | rs6551408 | 3 | 89353428 | T | C |
| 2188 | EPHA3 | rs6804377 | 3 | 89353777 | A | G |
| 2189 | EPHA3 | rs4857497 | 3 | 89376839 | T | G |
| 2190 | EPHA3 | rs1512912 | 3 | 89390621 | G | A |
| 2191 | EPHA3 | rs11916939 | 3 | 89392096 | G | A |
| 2192 | EPHA3 | rs993186 | 3 | 89397552 | A | C |
| 2193 | EPHA3 | rs3946805 | 3 | 89399066 | T | C |
| 2194 | EPHA3 | rs1040017 | 3 | 89412322 | A | C |
| 2195 | EPHA3 | rs12638746 | 3 | 89413745 | G | A |
| 2196 | EPHA3 | rs1512909 | 3 | 89419107 | G | A |
| 2197 | EPHA3 | rs12486528 | 3 | 89421084 | G | A |
| 2198 | EPHA3 | rs6768381 | 3 | 89440761 | T | C |
| 2199 | EPHA3 | rs9866959 | 3 | 89462625 | T | C |
| 2200 | EPHA3 | rs9833400 | 3 | 89472197 | A | G |
| 2201 | EPHA3 | rs9832305 | 3 | 89475468 | T | C |
| 2202 | EPHA3 | rs2063589 | 3 | 89485137 | C | T |
| 2203 | EPHA3 | rs9836340 | 3 | 89488444 | A | G |
| 2204 | EPHA3 | rs9842599 | 3 | 89489946 | A | G |
| 2205 | EPHA3 | rs17801001 | 3 | 89497245 | A | C |
| 2206 | EPHA3 | rs870898 | 3 | 89499754 | C | T |
| 2207 | EPHA3 | rs6782527 | 3 | 89525059 | C | T |
| 2208 | EPHA3 | rs7633500 | 3 | 89579772 | G | A |
| 2209 | EPHA3 | rs1398197 | 3 | 89581118 | C | T |
| 2210 | EPHA3 | rs1028011 | 3 | 89581309 | C | T |
| 2211 | EPHA3 | rs7626735 | 3 | 89589648 | C | A |
| 2212 | EPHA3 | rs6795074 | 3 | 89599342 | T | C |
| 2213 | EPHA3 | rs2117137 | 3 | 89608195 | T | C |
| 2214 | EPHA3 | rs11924356 | 3 | 89610077 | T | G |
| 2215 | EPHA3 | rs7632427 | 3 | 89617067 | T | C |
| 2216 | GSK3B | rs2873950 | 3 | 121029752 | A | C |
| 2217 | GSK3B | rs3108749 | 3 | 121077178 | C | T |
| 2218 | GSK3B | rs1719895 | 3 | 121078193 | C | T |
| 2219 | GSK3B | rs16830594 | 3 | 121088768 | A | G |
| 2220 | GSK3B | rs4340737 | 3 | 121104704 | C | T |
| 2221 | GSK3B | rs9872321 | 3 | 121110998 | C | T |
| 2222 | GSK3B | rs13314595 | 3 | 121113788 | C | T |
| 2223 | GSK3B | rs6438552 | 3 | 121114504 | A | G |
| 2224 | GSK3B | rs4072520 | 3 | 121118083 | C | A |
| 2225 | GSK3B | rs17810235 | 3 | 121157741 | C | T |
| 2226 | GSK3B | rs4688054 | 3 | 121216400 | T | C |
| 2227 | GSK3B | rs6770314 | 3 | 121225411 | G | A |
| 2228 | GSK3B | rs11919783 | 3 | 121229323 | G | A |
| 2229 | GSK3B | rs9834346 | 3 | 121236093 | A | G |
| 2230 | GSK3B | rs12630592 | 3 | 121250936 | G | T |
| 2231 | GSK3B | rs968824 | 3 | 121255730 | T | C |
| 2232 | GSK3B | rs2199503 | 3 | 121261179 | G | A |
| 2233 | SEMA5B | rs2303982 | 3 | 124113013 | G | T |
| 2234 | SEMA5B | rs2303983 | 3 | 124113036 | C | T |
| 2235 | SEMA5B | rs1011151 | 3 | 124121279 | G | A |
| 2236 | SEMA5B | rs4378918 | 3 | 124121699 | G | A |
| 2237 | SEMA5B | rs11923426 | 3 | 124123096 | C | A |
| 2238 | SEMA5B | rs2276780 | 3 | 124125091 | T | C |
| 2239 | SEMA5B | rs9846967 | 3 | 124125540 | C | T |
| 2240 | SEMA5B | rs17215029 | 3 | 124125856 | C | T |
| 2241 | SEMA5B | rs11710442 | 3 | 124126182 | C | A |
| 2242 | SEMA5B | rs2276777 | 3 | 124127967 | G | A |
| 2243 | SEMA5B | rs2276774 | 3 | 124129518 | T | C |
| 2244 | SEMA5B | rs9873067 | 3 | 124132212 | G | A |
| 2245 | SEMA5B | rs13099843 | 3 | 124133580 | A | C |
| 2246 | SEMA5B | rs11718661 | 3 | 124135223 | T | C |
| 2247 | SEMA5B | rs4677976 | 3 | 124138706 | C | A |
| 2248 | SEMA5B | rs2060090 | 3 | 124139894 | G | A |
| 2249 | SEMA5B | rs10934622 | 3 | 124142519 | A | G |
| 2250 | SEMA5B | rs6803981 | 3 | 124143377 | G | A |
| 2251 | SEMA5B | rs2332410 | 3 | 124144327 | G | A |
| 2252 | SEMA5B | rs11719594 | 3 | 124144553 | T | G |
| 2253 | SEMA5B | rs11719610 | 3 | 124144639 | A | G |
| 2254 | SEMA5B | rs2043172 | 3 | 124145445 | A | G |
| 2255 | SEMA5B | rs10755076 | 3 | 124146741 | A | G |
| 2256 | SEMA5B | rs9831044 | 3 | 124148738 | G | A |
| 2257 | SEMA5B | rs4677983 | 3 | 124151847 | G | A |
| 2258 | SEMA5B | rs10934626 | 3 | 124159213 | C | T |
| 2259 | SEMA5B | rs9839593 | 3 | 124159395 | T | C |
| 2260 | SEMA5B | rs4478030 | 3 | 124161197 | A | G |
| 2261 | SEMA5B | rs13094003 | 3 | 124162831 | A | G |
| 2262 | SEMA5B | rs17171 | 3 | 124164620 | G | A |
| 2263 | SEMA5B | rs4677872 | 3 | 124166729 | G | A |
| 2264 | SEMA5B | rs6438771 | 3 | 124166975 | G | A |
| 2265 | SEMA5B | rs6764971 | 3 | 124168803 | A | G |
| 2266 | SEMA5B | rs11717229 | 3 | 124169289 | T | C |
| 2267 | SEMA5B | rs9816759 | 3 | 124170860 | A | C |
| 2268 | SEMA5B | rs4234209 | 3 | 124176490 | G | A |
| 2269 | SEMA5B | rs895075 | 3 | 124177217 | A | G |
| 2270 | SEMA5B | rs9968193 | 3 | 124177620 | C | T |
| 2271 | SEMA5B | rs7432970 | 3 | 124177978 | G | A |
| 2272 | SEMA5B | rs1470399 | 3 | 124181451 | C | T |
| 2273 | SEMA5B | rs998945 | 3 | 124190123 | C | T |
| 2274 | SEMA5B | rs6769548 | 3 | 124192543 | C | T |
| 2275 | SEMA5B | rs1444820 | 3 | 124194034 | T | C |
| 2276 | SEMA5B | rs16833748 | 3 | 124196630 | A | G |
| 2277 | SEMA5B | rs2120806 | 3 | 124198502 | C | T |
| 2278 | SEMA5B | rs16833766 | 3 | 124203397 | A | G |
| 2279 | SEMA5B | rs11715232 | 3 | 124205881 | G | A |
| 2280 | SEMA5B | rs9874307 | 3 | 124207631 | G | A |
| 2281 | SEMA5B | rs2034177 | 3 | 124211779 | A | G |
| 2282 | SEMA5B | rs9864897 | 3 | 124213405 | G | T |
| 2283 | SEMA5B | rs9839656 | 3 | 124233393 | G | A |
| 2284 | SEMA5B | rs11922528 | 3 | 124234643 | C | T |
| 2285 | PLXNA1 | rs1347003 | 3 | 128214737 | A | G |
| 2286 | PLXNA1 | rs4679323 | 3 | 128219958 | A | C |
| 2287 | PLXNA1 | rs4679325 | 3 | 128224168 | C | T |
| 2288 | PLXNA1 | rs9851451 | 3 | 128229380 | A | G |
| 2289 | PLXNA1 | rs17751931 | 3 | 128239180 | G | A |
| 2290 | PLXNA1 | rs2116459 | 3 | 128240195 | C | T |
| 2291 | EPHB1 | rs3732568 | 3 | 135998026 | C | A |
| 2292 | EPHB1 | rs7648359 | 3 | 136006298 | C | T |
| 2293 | EPHB1 | rs9818551 | 3 | 136012398 | C | A |
| 2294 | EPHB1 | rs6439522 | 3 | 136013533 | C | T |
| 2295 | EPHB1 | rs4955502 | 3 | 136015474 | T | C |
| 2296 | EPHB1 | rs1913685 | 3 | 136019662 | G | A |
| 2297 | EPHB1 | rs885594 | 3 | 136034218 | T | C |
| 2298 | EPHB1 | rs1949305 | 3 | 136034403 | C | T |
| 2299 | EPHB1 | rs2138213 | 3 | 136036853 | T | C |
| 2300 | EPHB1 | rs3732569 | 3 | 136043482 | A | G |
| 2301 | EPHB1 | rs13091440 | 3 | 136050881 | G | A |
| 2302 | EPHB1 | rs12489360 | 3 | 136057409 | A | G |
| 2303 | EPHB1 | rs10935138 | 3 | 136057659 | G | T |
| 2304 | EPHB1 | rs9819889 | 3 | 136067454 | G | A |
| 2305 | EPHB1 | rs7651593 | 3 | 136067467 | A | C |
| 2306 | EPHB1 | rs6762261 | 3 | 136073828 | C | T |
| 2307 | EPHB1 | rs1515367 | 3 | 136075130 | G | T |
| 2308 | EPHB1 | rs1399709 | 3 | 136086267 | C | T |
| 2309 | EPHB1 | rs9868348 | 3 | 136089933 | C | T |
| 2310 | EPHB1 | rs6786490 | 3 | 136096278 | C | A |
| 2311 | EPHB1 | rs1996242 | 3 | 136101509 | T | C |
| 2312 | EPHB1 | rs16842240 | 3 | 136102414 | C | A |
| 2313 | EPHB1 | rs187586 | 3 | 136106877 | G | A |
| 2314 | EPHB1 | rs36221 | 3 | 136107330 | C | T |
| 2315 | EPHB1 | rs10512935 | 3 | 136107945 | G | A |
| 2316 | EPHB1 | rs36218 | 3 | 136109029 | C | T |
| 2317 | EPHB1 | rs1007557 | 3 | 136111469 | T | C |
| 2318 | EPHB1 | rs9682984 | 3 | 136115211 | G | A |
| 2319 | EPHB1 | rs7651682 | 3 | 136117008 | T | C |
| 2320 | EPHB1 | rs11709198 | 3 | 136119214 | A | C |
| 2321 | EPHB1 | rs36198 | 3 | 136119486 | G | A |
| 2322 | EPHB1 | rs11719058 | 3 | 136120636 | C | T |
| 2323 | EPHB1 | rs11920780 | 3 | 136125357 | T | C |
| 2324 | EPHB1 | rs36182 | 3 | 136128400 | A | C |
| 2325 | EPHB1 | rs40437 | 3 | 136133622 | T | C |
| 2326 | EPHB1 | rs36161 | 3 | 136138979 | T | C |
| 2327 | EPHB1 | rs6788931 | 3 | 136139314 | G | A |
| 2328 | EPHB1 | rs984709 | 3 | 136140542 | G | A |
| 2329 | EPHB1 | rs9874260 | 3 | 136142241 | T | C |
| 2330 | EPHB1 | rs983401 | 3 | 136144120 | A | C |
| 2331 | EPHB1 | rs972234 | 3 | 136151449 | C | T |
| 2332 | EPHB1 | rs4955460 | 3 | 136151931 | T | C |
| 2333 | EPHB1 | rs16842348 | 3 | 136153847 | A | C |
| 2334 | EPHB1 | rs9815290 | 3 | 136154449 | G | A |
| 2335 | EPHB1 | rs36132 | 3 | 136158766 | C | T |
| 2336 | EPHB1 | rs6781640 | 3 | 136164137 | T | C |
| 2337 | EPHB1 | rs17282355 | 3 | 136179021 | C | T |
| 2338 | EPHB1 | rs39699 | 3 | 136180874 | C | T |
| 2339 | EPHB1 | rs6776069 | 3 | 136181559 | T | C |
| 2340 | EPHB1 | rs9855772 | 3 | 136187702 | T | C |
| 2341 | EPHB1 | rs7613213 | 3 | 136192641 | C | T |
| 2342 | EPHB1 | rs40524 | 3 | 136201828 | C | T |
| 2343 | EPHB1 | rs36073 | 3 | 136202844 | G | A |
| 2344 | EPHB1 | rs41182 | 3 | 136208971 | C | T |
| 2345 | EPHB1 | rs36064 | 3 | 136210523 | T | C |
| 2346 | EPHB1 | rs36059 | 3 | 136215422 | G | T |
| 2347 | EPHB1 | rs40347 | 3 | 136216206 | A | C |
| 2348 | EPHB1 | rs11720128 | 3 | 136216594 | C | T |
| 2349 | EPHB1 | rs36057 | 3 | 136219361 | A | C |
| 2350 | EPHB1 | rs10935153 | 3 | 136235615 | C | A |
| 2351 | EPHB1 | rs36039 | 3 | 136238281 | C | A |
| 2352 | EPHB1 | rs6791224 | 3 | 136246150 | G | A |
| 2353 | EPHB1 | rs6793611 | 3 | 136246207 | G | A |
| 2354 | EPHB1 | rs36041 | 3 | 136248988 | C | T |
| 2355 | EPHB1 | rs253876 | 3 | 136251605 | A | G |
| 2356 | EPHB1 | rs12639432 | 3 | 136253210 | C | T |
| 2357 | EPHB1 | rs6439552 | 3 | 136275534 | C | T |
| 2358 | EPHB1 | rs6439553 | 3 | 136288271 | C | A |
| 2359 | EPHB1 | rs959562 | 3 | 136288462 | T | C |
| 2360 | EPHB1 | rs2030737 | 3 | 136292067 | A | G |
| 2361 | EPHB1 | rs6764345 | 3 | 136298082 | T | C |
| 2362 | EPHB1 | rs9847974 | 3 | 136299518 | A | C |
| 2363 | EPHB1 | rs11711351 | 3 | 136303819 | G | A |
| 2364 | EPHB1 | rs7372127 | 3 | 136304089 | A | C |
| 2365 | EPHB1 | rs7374961 | 3 | 136308678 | A | G |
| 2366 | EPHB1 | rs1554675 | 3 | 136309607 | A | G |
| 2367 | EPHB1 | rs7374193 | 3 | 136312228 | G | A |
| 2368 | EPHB1 | rs9813334 | 3 | 136312252 | A | G |
| 2369 | EPHB1 | rs10935154 | 3 | 136313222 | C | T |
| 2370 | EPHB1 | rs13074164 | 3 | 136319364 | A | G |
| 2371 | EPHB1 | rs9864760 | 3 | 136319729 | G | A |
| 2372 | EPHB1 | rs16842632 | 3 | 136320305 | G | A |
| 2373 | EPHB1 | rs11706256 | 3 | 136324142 | T | C |
| 2374 | EPHB1 | rs940639 | 3 | 136325113 | C | T |
| 2375 | EPHB1 | rs6805145 | 3 | 136325355 | C | T |
| 2376 | EPHB1 | rs2140766 | 3 | 136325976 | T | C |
| 2377 | EPHB1 | rs7625747 | 3 | 136328383 | T | G |
| 2378 | EPHB1 | rs7626314 | 3 | 136328975 | T | C |
| 2379 | EPHB1 | rs13076510 | 3 | 136331543 | A | G |
| 2380 | EPHB1 | rs1404577 | 3 | 136331641 | C | T |
| 2381 | EPHB1 | rs940638 | 3 | 136333461 | A | G |
| 2382 | EPHB1 | rs6772933 | 3 | 136335379 | A | G |
| 2383 | EPHB1 | rs11713704 | 3 | 136336452 | C | T |
| 2384 | EPHB1 | rs12488683 | 3 | 136337633 | G | A |
| 2385 | EPHB1 | rs868909 | 3 | 136342524 | C | T |
| 2386 | EPHB1 | rs7373413 | 3 | 136344369 | G | A |
| 2387 | EPHB1 | rs3772647 | 3 | 136347219 | T | C |
| 2388 | EPHB1 | rs3821504 | 3 | 136347568 | T | G |
| 2389 | EPHB1 | rs3772646 | 3 | 136350642 | G | A |
| 2390 | EPHB1 | rs6764768 | 3 | 136352748 | C | T |
| 2391 | EPHB1 | rs12492126 | 3 | 136354265 | A | G |
| 2392 | EPHB1 | rs2291989 | 3 | 136355230 | C | T |
| 2393 | EPHB1 | rs1501205 | 3 | 136362244 | T | C |
| 2394 | EPHB1 | rs3772643 | 3 | 136362311 | G | A |
| 2395 | EPHB1 | rs905048 | 3 | 136364979 | A | G |
| 2396 | EPHB1 | rs3772638 | 3 | 136367163 | G | A |
| 2397 | EPHB1 | rs6801620 | 3 | 136375176 | C | T |
| 2398 | EPHB1 | rs4894252 | 3 | 136375373 | C | A |
| 2399 | EPHB1 | rs9822213 | 3 | 136379409 | A | G |
| 2400 | EPHB1 | rs10512945 | 3 | 136381650 | G | A |
| 2401 | EPHB1 | rs16842755 | 3 | 136383073 | T | C |
| 2402 | EPHB1 | rs1875371 | 3 | 136383637 | G | A |
| 2403 | EPHB1 | rs9816567 | 3 | 136385157 | G | A |
| 2404 | EPHB1 | rs4894287 | 3 | 136390975 | C | T |
| 2405 | EPHB1 | rs17710899 | 3 | 136394881 | C | T |
| 2406 | EPHB1 | rs16842783 | 3 | 136396330 | G | A |
| 2407 | EPHB1 | rs3772637 | 3 | 136396640 | C | T |
| 2408 | EPHB1 | rs6763009 | 3 | 136397878 | C | A |
| 2409 | EPHB1 | rs7622312 | 3 | 136401212 | C | T |
| 2410 | EPHB1 | rs6439563 | 3 | 136411407 | G | A |
| 2411 | EPHB1 | rs931726 | 3 | 136413630 | C | T |
| 2412 | EPHB1 | rs4309752 | 3 | 136416306 | G | A |
| 2413 | EPHB1 | rs9858763 | 3 | 136419273 | G | A |
| 2414 | EPHB1 | rs6802560 | 3 | 136419965 | T | G |
| 2415 | EPHB1 | rs1392920 | 3 | 136424647 | G | A |
| 2416 | EPHB1 | rs11917038 | 3 | 136427814 | C | T |
| 2417 | EPHB1 | rs4894320 | 3 | 136428257 | G | A |
| 2418 | EPHB1 | rs7613488 | 3 | 136428556 | A | G |
| 2419 | EPHB1 | rs9871429 | 3 | 136433726 | C | A |
| 2420 | EPHB1 | rs8179953 | 3 | 136437015 | G | A |
| 2421 | EPHB1 | rs9865620 | 3 | 136438405 | G | A |
| 2422 | EPHB1 | rs6806284 | 3 | 136438602 | A | G |
| 2423 | EPHB1 | rs6782829 | 3 | 136438993 | G | A |
| 2424 | EPHB1 | rs11719703 | 3 | 136440351 | A | G |
| 2425 | EPHB1 | rs6793828 | 3 | 136443160 | T | C |
| 2426 | EPHB1 | rs11717042 | 3 | 136450058 | G | A |
| 2427 | EPHB1 | rs4456886 | 3 | 136452627 | C | T |
| 2428 | EPHB1 | rs13085606 | 3 | 136459854 | T | C |
| 2429 | EPHB1 | rs16842883 | 3 | 136464085 | A | G |
| 2430 | NCK1 | rs9846691 | 3 | 138088961 | C | T |
| 2431 | NCK1 | rs7648198 | 3 | 138154451 | A | C |
| 2432 | MRAS | rs1199338 | 3 | 139570157 | T | G |
| 2433 | MRAS | rs1199334 | 3 | 139573830 | C | T |
| 2434 | MRAS | rs9289559 | 3 | 139573879 | G | A |
| 2435 | MRAS | rs1199333 | 3 | 139574391 | A | C |
| 2436 | MRAS | rs253662 | 3 | 139580003 | C | T |
| 2437 | MRAS | rs1720819 | 3 | 139583229 | A | C |
| 2438 | MRAS | rs1209710 | 3 | 139589388 | A | G |
| 2439 | MRAS | rs2306374 | 3 | 139602642 | T | C |
| 2440 | MRAS | rs40593 | 3 | 139605471 | A | G |
| 2441 | EPHB3 | rs4132006 | 3 | 185764350 | C | T |
| 2442 | EPHB3 | rs9647371 | 3 | 185766973 | C | T |
| 2443 | EPHB3 | rs9862375 | 3 | 185773042 | G | A |
| 2444 | EPHB3 | rs9823034 | 3 | 185776304 | T | C |
| 2445 | EPHB3 | rs7652280 | 3 | 185785328 | C | T |
| 2446 | PAK2 | rs3844057 | 3 | 197946898 | T | C |
| 2447 | PAK2 | rs1357287 | 3 | 197969364 | C | T |
| 2448 | PAK2 | rs1798619 | 3 | 197970248 | T | G |
| 2449 | PAK2 | rs9872035 | 3 | 197979099 | T | C |
| 2450 | PAK2 | rs2089979 | 3 | 197985810 | A | G |
| 2451 | PAK2 | rs9325377 | 3 | 197990550 | A | G |
| 2452 | PAK2 | rs1718414 | 3 | 197999033 | A | G |
| 2453 | PAK2 | rs7646247 | 3 | 198003606 | C | T |
| 2454 | PAK2 | rs6583176 | 3 | 198009975 | G | A |
| 2455 | PAK2 | rs11185455 | 3 | 198025977 | A | G |
| 2456 | PAK2 | rs10446497 | 3 | 198030926 | T | G |
| 2457 | PAK2 | rs6583177 | 3 | 198034139 | G | T |
| 2458 | PAK2 | rs2084385 | 3 | 198038264 | G | A |
| 2459 | PAK2 | rs15614 | 3 | 198043346 | A | G |
| 2460 | ABLIM2 | rs4696709 | 4 | 8017712 | G | A |
| 2461 | ABLIM2 | rs4696886 | 4 | 8018583 | C | T |
| 2462 | ABLIM2 | rs10008860 | 4 | 8020494 | G | A |
| 2463 | ABLIM2 | rs7699715 | 4 | 8031552 | C | T |
| 2464 | ABLIM2 | rs870714 | 4 | 8032138 | G | A |
| 2465 | ABLIM2 | rs1574393 | 4 | 8035118 | C | T |
| 2466 | ABLIM2 | rs13133269 | 4 | 8035636 | T | C |
| 2467 | ABLIM2 | rs7686189 | 4 | 8036524 | C | T |
| 2468 | ABLIM2 | rs7677869 | 4 | 8037760 | C | T |
| 2469 | ABLIM2 | rs4696725 | 4 | 8038457 | G | A |
| 2470 | ABLIM2 | rs4696662 | 4 | 8042487 | T | C |
| 2471 | ABLIM2 | rs4696726 | 4 | 8043532 | A | G |
| 2472 | ABLIM2 | rs13129405 | 4 | 8043649 | C | T |
| 2473 | ABLIM2 | rs4696727 | 4 | 8043686 | T | C |
| 2474 | ABLIM2 | rs17772026 | 4 | 8048542 | A | G |
| 2475 | ABLIM2 | rs16841542 | 4 | 8053291 | T | G |
| 2476 | ABLIM2 | rs4696664 | 4 | 8059164 | G | A |
| 2477 | ABLIM2 | rs17180256 | 4 | 8060659 | A | G |
| 2478 | ABLIM2 | rs13125250 | 4 | 8061012 | T | C |
| 2479 | ABLIM2 | rsl317368 | 4 | 8062596 | T | G |
| 2480 | ABLIM2 | rs16841559 | 4 | 8063067 | C | T |
| 2481 | ABLIM2 | rs4696729 | 4 | 8066321 | C | T |
| 2482 | ABLIM2 | rs16841570 | 4 | 8067064 | T | C |
| 2483 | ABLIM2 | rs9990943 | 4 | 8068024 | C | T |
| 2484 | ABLIM2 | rs2385895 | 4 | 8068659 | G | T |
| 2485 | ABLIM2 | rs17795113 | 4 | 8069056 | C | T |
| 2486 | ABLIM2 | rs4696666 | 4 | 8070347 | G | A |
| 2487 | ABLIM2 | rs11932811 | 4 | 8071402 | A | G |
| 2488 | ABLIM2 | rs13131680 | 4 | 8072492 | C | A |
| 2489 | ABLIM2 | rs7685070 | 4 | 8074384 | G | A |
| 2490 | ABLIM2 | rs11729381 | 4 | 8077076 | C | T |
| 2491 | ABLIM2 | rs11726230 | 4 | 8077138 | G | T |
| 2492 | ABLIM2 | rs17795172 | 4 | 8079837 | G | A |
| 2493 | ABLIM2 | rs2385904 | 4 | 8082371 | G | A |
| 2494 | ABLIM2 | rs11942146 | 4 | 8082490 | T | G |
| 2495 | ABLIM2 | rs4696732 | 4 | 8084288 | C | T |
| 2496 | ABLIM2 | rs6857972 | 4 | 8085672 | C | T |
| 2497 | ABLIM2 | rs1574131 | 4 | 8092700 | A | G |
| 2498 | ABLIM2 | rs6852834 | 4 | 8096369 | A | G |
| 2499 | ABLIM2 | rs4696738 | 4 | 8096392 | G | A |
| 2500 | ABLIM2 | rs2385916 | 4 | 8098873 | T | C |
| 2501 | ABLIM2 | rs6849076 | 4 | 8100613 | A | G |
| 2502 | ABLIM2 | rs1320062 | 4 | 8100797 | G | A |
| 2503 | ABLIM2 | rs1574414 | 4 | 8100956 | G | T |
| 2504 | ABLIM2 | rs10029338 | 4 | 8101243 | G | A |
| 2505 | ABLIM2 | rs13152591 | 4 | 8104705 | T | G |
| 2506 | ABLIM2 | rs10222845 | 4 | 8105372 | G | A |
| 2507 | ABLIM2 | rs2076769 | 4 | 8112119 | G | A |
| 2508 | ABLIM2 | rs4696669 | 4 | 8113102 | A | G |
| 2509 | ABLIM2 | rs16841624 | 4 | 8113730 | C | T |
| 2510 | ABLIM2 | rs6845573 | 4 | 8114401 | C | T |
| 2511 | ABLIM2 | rs17178569 | 4 | 8115661 | G | A |
| 2512 | ABLIM2 | rs6829649 | 4 | 8121693 | T | G |
| 2513 | ABLIM2 | rs6842276 | 4 | 8126034 | C | T |
| 2514 | ABLIM2 | rs6856548 | 4 | 8131381 | C | T |
| 2515 | ABLIM2 | rs6447849 | 4 | 8135460 | C | T |
| 2516 | ABLIM2 | rs11724908 | 4 | 8136074 | G | A |
| 2517 | ABLIM2 | rs2292739 | 4 | 8140498 | C | T |
| 2518 | ABLIM2 | rs4303965 | 4 | 8151172 | A | G |
| 2519 | ABLIM2 | rs10938688 | 4 | 8153532 | C | T |
| 2520 | ABLIM2 | rs4696758 | 4 | 8157149 | G | A |
| 2521 | ABLIM2 | rs1878519 | 4 | 8161682 | C | T |
| 2522 | ABLIM2 | rs1105616 | 4 | 8162017 | T | G |
| 2523 | ABLIM2 | rs2138826 | 4 | 8167102 | G | A |
| 2524 | ABLIM2 | rs1125307 | 4 | 8168571 | C | T |
| 2525 | ABLIM2 | rs10938692 | 4 | 8169461 | T | C |
| 2526 | ABLIM2 | rs1517525 | 4 | 8170837 | C | T |
| 2527 | ABLIM2 | rs13122226 | 4 | 8171020 | T | C |
| 2528 | ABLIM2 | rs4696763 | 4 | 8171566 | A | G |
| 2529 | ABLIM2 | rs10002315 | 4 | 8171705 | C | T |
| 2530 | ABLIM2 | rs13144749 | 4 | 8172405 | G | A |
| 2531 | ABLIM2 | rs17181631 | 4 | 8172699 | C | T |
| 2532 | ABLIM2 | rs11936149 | 4 | 8173396 | T | G |
| 2533 | ABLIM2 | rs4696765 | 4 | 8176688 | T | C |
| 2534 | ABLIM2 | rs11724027 | 4 | 8178072 | C | T |
| 2535 | ABLIM2 | rs1104756 | 4 | 8181758 | A | C |
| 2536 | ABLIM2 | rs1878521 | 4 | 8189545 | T | G |
| 2537 | ABLIM2 | rs4422405 | 4 | 8190858 | A | G |
| 2538 | ABLIM2 | rs16841848 | 4 | 8193572 | C | T |
| 2539 | ABLIM2 | rs4696769 | 4 | 8199059 | G | A |
| 2540 | ABLIM2 | rs12506374 | 4 | 8205434 | T | C |
| 2541 | ABLIM2 | rs7660199 | 4 | 8208610 | A | G |
| 2542 | ABLIM2 | rs13132877 | 4 | 8214460 | T | G |
| 2543 | SLIT2 | rs564041 | 4 | 19861365 | T | C |
| 2544 | SLIT2 | rs7655084 | 4 | 19864404 | T | G |
| 2545 | SLIT2 | rs17534458 | 4 | 19870884 | G | A |
| 2546 | SLIT2 | rs7664347 | 4 | 19874633 | C | T |
| 2547 | SLIT2 | rs587032 | 4 | 19880871 | T | C |
| 2548 | SLIT2 | rs16869461 | 4 | 19884575 | A | — |
| 2549 | SLIT2 | rs6447952 | 4 | 19885350 | G | A |
| 2550 | SLIT2 | rs7680377 | 4 | 19888956 | T | G |
| 2551 | SLIT2 | rs16869481 | 4 | 19891432 | T | — |
| 2552 | SLIT2 | rs17601458 | 4 | 19892167 | C | T |
| 2553 | SLIT2 | rs6828506 | 4 | 19900021 | T | C |
| 2554 | SLIT2 | rs593943 | 4 | 19901337 | A | G |
| 2555 | SLIT2 | rs12233914 | 4 | 19906678 | A | G |
| 2556 | SLIT2 | rs624354 | 4 | 19909290 | C | T |
| 2557 | SLIT2 | rs13140329 | 4 | 19917020 | C | T |
| 2558 | SLIT2 | rs1323062 | 4 | 19918840 | G | A |
| 2559 | SLIT2 | rs13434430 | 4 | 19919271 | T | C |
| 2560 | SLIT2 | rs7668974 | 4 | 19921095 | C | T |
| 2561 | SLIT2 | rs12646142 | 4 | 19927935 | T | C |
| 2562 | SLIT2 | rs9993087 | 4 | 19931887 | A | C |
| 2563 | SLIT2 | rs4696953 | 4 | 19932011 | C | A |
| 2564 | SLIT2 | rs10938796 | 4 | 19936108 | C | A |
| 2565 | SLIT2 | rs12503652 | 4 | 19942820 | T | C |
| 2566 | SLIT2 | rs6447955 | 4 | 19957037 | G | A |
| 2567 | SLIT2 | rs6447958 | 4 | 19970940 | G | A |
| 2568 | SLIT2 | rs4368605 | 4 | 19971281 | G | A |
| 2569 | SLIT2 | rs4406013 | 4 | 19971814 | C | T |
| 2570 | SLIT2 | rs5016268 | 4 | 19979241 | A | G |
| 2571 | SLIT2 | rs13142826 | 4 | 20000713 | G | A |
| 2572 | SLIT2 | rs7435615 | 4 | 20008266 | G | A |
| 2573 | SLIT2 | rs9996157 | 4 | 20014130 | C | T |
| 2574 | SLIT2 | rs6813778 | 4 | 20025403 | A | G |
| 2575 | SLIT2 | rs4697165 | 4 | 20027406 | C | T |
| 2576 | SLIT2 | rs17537997 | 4 | 20042850 | G | A |
| 2577 | SLIT2 | rs16869595 | 4 | 20046230 | G | A |
| 2578 | SLIT2 | rs6849536 | 4 | 20049574 | C | T |
| 2579 | SLIT2 | rs884770 | 4 | 20056138 | A | G |
| 2580 | SLIT2 | rs12512717 | 4 | 20058863 | A | G |
| 2581 | SLIT2 | rs16869615 | 4 | 20063982 | T | C |
| 2582 | SLIT2 | rs9990844 | 4 | 20065053 | A | C |
| 2583 | SLIT2 | rs11733205 | 4 | 20067786 | T | C |
| 2584 | SLIT2 | rs11942897 | 4 | 20068216 | C | T |
| 2585 | SLIT2 | rs1033111 | 4 | 20076865 | C | T |
| 2586 | SLIT2 | rs11936122 | 4 | 20082376 | G | T |
| 2587 | SLIT2 | rs1902845 | 4 | 20082897 | T | C |
| 2588 | SLIT2 | rs491795 | 4 | 20087382 | T | C |
| 2589 | SLIT2 | rs17612037 | 4 | 20093354 | A | G |
| 2590 | SLIT2 | rs16869665 | 4 | 20095328 | G | A |
| 2591 | SLIT2 | rs569230 | 4 | 20098122 | T | C |
| 2592 | SLIT2 | rs4443280 | 4 | 20099765 | G | A |
| 2593 | SLIT2 | rs522501 | 4 | 20106375 | T | C |
| 2594 | SLIT2 | rs12374415 | 4 | 20106838 | C | T |
| 2595 | SLIT2 | rs490478 | 4 | 20107582 | G | A |
| 2596 | SLIT2 | rs729918 | 4 | 20115199 | G | A |
| 2597 | SLIT2 | rs6810790 | 4 | 20124768 | A | G |
| 2598 | SLIT2 | rs6447967 | 4 | 20125439 | T | C |
| 2599 | SLIT2 | rs6825654 | 4 | 20127701 | A | C |
| 2600 | SLIT2 | rs519813 | 4 | 20130091 | G | A |
| 2601 | SLIT2 | rs7690660 | 4 | 20136962 | T | G |
| 2602 | SLIT2 | rs3775815 | 4 | 20138768 | G | T |
| 2603 | SLIT2 | rs9992559 | 4 | 20139472 | T | C |
| 2604 | SLIT2 | rs2168801 | 4 | 20140079 | G | A |
| 2605 | SLIT2 | rs2322557 | 4 | 20142112 | G | A |
| 2606 | SLIT2 | rs6816854 | 4 | 20143565 | C | T |
| 2607 | SLIT2 | rs573118 | 4 | 20143784 | T | C |
| 2608 | SLIT2 | rs3775816 | 4 | 20144815 | T | C |
| 2609 | SLIT2 | rs520535 | 4 | 20144974 | A | G |
| 2610 | SLIT2 | rs6849986 | 4 | 20146631 | G | A |
| 2611 | SLIT2 | rs7658163 | 4 | 20148427 | G | A |
| 2612 | SLIT2 | rs3775820 | 4 | 20149638 | G | A |
| 2613 | SLIT2 | rs17613956 | 4 | 20151201 | C | T |
| 2614 | SLIT2 | rs16869739 | 4 | 20152807 | A | G |
| 2615 | SLIT2 | rs16869740 | 4 | 20152890 | G | A |
| 2616 | SLIT2 | rs675465 | 4 | 20152915 | A | G |
| 2617 | SLIT2 | rs7669094 | 4 | 20153514 | T | G |
| 2618 | SLIT2 | rs3775824 | 4 | 20154010 | G | T |
| 2619 | SLIT2 | rs3775827 | 4 | 20154412 | G | A |
| 2620 | SLIT2 | rs12501787 | 4 | 20156630 | C | A |
| 2621 | SLIT2 | rs2292439 | 4 | 20156936 | G | A |
| 2622 | SLIT2 | rs11936468 | 4 | 20158074 | T | C |
| 2623 | SLIT2 | rs17542486 | 4 | 20161608 | A | C |
| 2624 | SLIT2 | rs575088 | 4 | 20166557 | A | C |
| 2625 | SLIT2 | rs13328045 | 4 | 20170208 | C | T |
| 2626 | SLIT2 | rs7692811 | 4 | 20171178 | G | A |
| 2627 | SLIT2 | rs10516357 | 4 | 20172206 | G | A |
| 2628 | SLIT2 | rs506358 | 4 | 20174959 | C | T |
| 2629 | SLIT2 | rs1870260 | 4 | 20175964 | G | A |
| 2630 | SLIT2 | rs17620615 | 4 | 20179885 | G | A |
| 2631 | SLIT2 | rs644607 | 4 | 20180434 | C | T |
| 2632 | SLIT2 | rs7666974 | 4 | 20188470 | G | T |
| 2633 | SLIT2 | rs2168802 | 4 | 20194981 | C | T |
| 2634 | SLIT2 | rs10010466 | 4 | 20210409 | T | G |
| 2635 | SLIT2 | rs2322559 | 4 | 20214692 | A | G |
| 2636 | SLIT2 | rs1350400 | 4 | 20222728 | A | G |
| 2637 | SLIT2 | rs17544103 | 4 | 20228988 | C | T |
| 2638 | SLIT2 | rs1379659 | 4 | 20229781 | A | G |
| 2639 | SLIT2 | rs8663 | 4 | 20230788 | G | A |
| 2640 | SLIT2 | rs1457916 | 4 | 20234787 | G | A |
| 2641 | EPHA5 | rs4362877 | 4 | 65877041 | A | C |
| 2642 | EPHA5 | rs11938753 | 4 | 65879864 | C | T |
| 2643 | EPHA5 | rs7349683 | 4 | 65880399 | C | T |
| 2644 | EPHA5 | rs7678888 | 4 | 65880654 | A | G |
| 2645 | EPHA5 | rs11945873 | 4 | 65881469 | A | G |
| 2646 | EPHA5 | rs986208 | 4 | 65889087 | T | C |
| 2647 | EPHA5 | rs1349446 | 4 | 65900929 | G | A |
| 2648 | EPHA5 | rs1901264 | 4 | 65910337 | A | G |
| 2649 | EPHA5 | rs10012882 | 4 | 65925144 | T | C |
| 2650 | EPHA5 | rs2034537 | 4 | 65926964 | G | A |
| 2651 | EPHA5 | rs17752325 | 4 | 65927951 | C | T |
| 2652 | EPHA5 | rs1451173 | 4 | 65929033 | A | G |
| 2653 | EPHA5 | rs1376416 | 4 | 65940868 | A | G |
| 2654 | EPHA5 | rs6828375 | 4 | 65943121 | A | G |
| 2655 | EPHA5 | rs7683477 | 4 | 65944615 | A | G |
| 2656 | EPHA5 | rs2084682 | 4 | 65944644 | A | G |
| 2657 | EPHA5 | rs1597611 | 4 | 65946897 | G | A |
| 2658 | EPHA5 | rs2349724 | 4 | 65947502 | T | C |
| 2659 | EPHA5 | rs1376412 | 4 | 65949610 | A | G |
| 2660 | EPHA5 | rs2198103 | 4 | 65954774 | T | C |
| 2661 | EPHA5 | rs13120093 | 4 | 65955006 | C | T |
| 2662 | EPHA5 | rs2305351 | 4 | 65962766 | C | T |
| 2663 | EPHA5 | rs17086224 | 4 | 65964956 | T | C |
| 2664 | EPHA5 | rs1451170 | 4 | 65965779 | A | G |
| 2665 | EPHA5 | rs1451171 | 4 | 65965921 | A | C |
| 2666 | EPHA5 | rs7678554 | 4 | 65970811 | A | G |
| 2667 | EPHA5 | rs7672488 | 4 | 65977244 | A | G |
| 2668 | EPHA5 | rs6831652 | 4 | 65983020 | T | C |
| 2669 | EPHA5 | rs10013782 | 4 | 65987557 | A | G |
| 2670 | EPHA5 | rs10003638 | 4 | 65987745 | C | A |
| 2671 | EPHA5 | rs10007066 | 4 | 65989016 | G | A |
| 2672 | EPHA5 | rs10006892 | 4 | 65991076 | A | G |
| 2673 | EPHA5 | rs9991256 | 4 | 66010449 | G | A |
| 2674 | EPHA5 | rs4437303 | 4 | 66015514 | G | T |
| 2675 | EPHA5 | rs4425430 | 4 | 66023562 | A | G |
| 2676 | EPHA5 | rs4637457 | 4 | 66026265 | T | G |
| 2677 | EPHA5 | rs11936348 | 4 | 66027549 | C | T |
| 2678 | EPHA5 | rs4640717 | 4 | 66029475 | T | G |
| 2679 | EPHA5 | rs7694306 | 4 | 66031143 | C | T |
| 2680 | EPHA5 | rs11724363 | 4 | 66037699 | G | A |
| 2681 | EPHA5 | rs4337775 | 4 | 66042172 | G | A |
| 2682 | EPHA5 | rs6815080 | 4 | 66045082 | C | T |
| 2683 | EPHA5 | rs7654891 | 4 | 66057228 | A | G |
| 2684 | EPHA5 | rs4467633 | 4 | 66062315 | C | T |
| 2685 | EPHA5 | rs13137977 | 4 | 66063134 | C | A |
| 2686 | EPHA5 | rs12504386 | 4 | 66063458 | C | T |
| 2687 | EPHA5 | rs4132313 | 4 | 66064176 | T | C |
| 2688 | EPHA5 | rs7679950 | 4 | 66065878 | C | T |
| 2689 | EPHA5 | rs11131599 | 4 | 66066025 | G | A |
| 2690 | EPHA5 | rs11731307 | 4 | 66071596 | C | T |
| 2691 | EPHA5 | rs7684022 | 4 | 66072296 | A | G |
| 2692 | EPHA5 | rs10032173 | 4 | 66079709 | A | G |
| 2693 | EPHA5 | rs6551933 | 4 | 66086162 | G | A |
| 2694 | EPHA5 | rs13134593 | 4 | 66093688 | A | C |
| 2695 | EPHA5 | rs4518304 | 4 | 66104261 | G | A |
| 2696 | EPHA5 | rs11931601 | 4 | 66114504 | T | C |
| 2697 | EPHA5 | rs9994092 | 4 | 66118709 | C | A |
| 2698 | EPHA5 | rs4241667 | 4 | 66123662 | A | G |
| 2699 | EPHA5 | rs13127631 | 4 | 66126469 | G | T |
| 2700 | EPHA5 | rs12506403 | 4 | 66136033 | C | A |
| 2701 | EPHA5 | rs6854088 | 4 | 66138625 | A | G |
| 2702 | EPHA5 | rs4624709 | 4 | 66152139 | G | T |
| 2703 | EPHA5 | rs4404602 | 4 | 66153408 | T | C |
| 2704 | EPHA5 | rs6816785 | 4 | 66157876 | A | C |
| 2705 | EPHA5 | rs4320186 | 4 | 66166188 | G | A |
| 2706 | EPHA5 | rs7697951 | 4 | 66169736 | T | C |
| 2707 | EPHA5 | rs4422467 | 4 | 66177240 | G | A |
| 2708 | EPHA5 | rs4611973 | 4 | 66198991 | G | A |
| 2709 | EPHA5 | rs4441811 | 4 | 66204084 | C | T |
| 2710 | MASA | rs6826022 | 4 | 83568002 | T | C |
| 2711 | MASA | rs8732 | 4 | 83570845 | A | G |
| 2712 | MASA | rs10516674 | 4 | 83577786 | A | G |
| 2713 | MASA | rs1878494 | 4 | 83581216 | A | G |
| 2714 | MASA | rs4285076 | 4 | 83588311 | G | A |
| 2715 | MASA | rs1980187 | 4 | 83597896 | A | C |
| 2716 | UNC5C | rs1813074 | 4 | 96306996 | A | C |
| 2717 | UNC5C | rs17415748 | 4 | 96322768 | C | T |
| 2718 | UNC5C | rs2289043 | 4 | 96325345 | C | T |
| 2719 | UNC5C | rs3796462 | 4 | 96327852 | A | G |
| 2720 | UNC5C | rs3775059 | 4 | 96336253 | A | C |
| 2721 | UNC5C | rs11737434 | 4 | 96341638 | C | T |
| 2722 | UNC5C | rs3775053 | 4 | 96342489 | G | A |
| 2723 | UNC5C | rs1865378 | 4 | 96353862 | C | A |
| 2724 | UNC5C | rs2571037 | 4 | 96357206 | T | C |
| 2725 | UNC5C | rs2865384 | 4 | 96364143 | T | C |
| 2726 | UNC5C | rs6850673 | 4 | 96366883 | T | C |
| 2727 | UNC5C | rs10516960 | 4 | 96378700 | T | G |
| 2728 | UNC5C | rs12643654 | 4 | 96378840 | A | G |
| 2729 | UNC5C | rs4699412 | 4 | 96379140 | T | C |
| 2730 | UNC5C | rs7434638 | 4 | 96380365 | T | C |
| 2731 | UNC5C | rs11730446 | 4 | 96382267 | A | G |
| 2732 | UNC5C | rs2865414 | 4 | 96387742 | A | G |
| 2733 | UNC5C | rs4699837 | 4 | 96389462 | T | C |
| 2734 | UNC5C | rs3775003 | 4 | 96390234 | G | A |
| 2735 | UNC5C | rs7674595 | 4 | 96398037 | A | G |
| 2736 | UNC5C | rs6838035 | 4 | 96398773 | A | C |
| 2737 | UNC5C | rs17352477 | 4 | 96399583 | T | C |
| 2738 | UNC5C | rs4254782 | 4 | 96400057 | A | G |
| 2739 | UNC5C | rs12640070 | 4 | 96402555 | C | T |
| 2740 | UNC5C | rs17352777 | 4 | 96406384 | C | T |
| 2741 | UNC5C | rs7658435 | 4 | 96407526 | G | T |
| 2742 | UNC5C | rs6847919 | 4 | 96408836 | T | C |
| 2743 | UNC5C | rs2865416 | 4 | 96412722 | T | G |
| 2744 | UNC5C | rs6820369 | 4 | 96421245 | A | G |
| 2745 | UNC5C | rs11936554 | 4 | 96421807 | C | T |
| 2746 | UNC5C | rs10516962 | 4 | 96422401 | G | A |
| 2747 | UNC5C | rs10516963 | 4 | 96422541 | C | T |
| 2748 | UNC5C | rs11097465 | 4 | 96423579 | C | T |
| 2749 | UNC5C | rs4699840 | 4 | 96427777 | T | C |
| 2750 | UNC5C | rs4504262 | 4 | 96428827 | G | T |
| 2751 | UNC5C | rs6857589 | 4 | 96432906 | C | T |
| 2752 | UNC5C | rs6831808 | 4 | 96432951 | G | A |
| 2753 | UNC5C | rs17433092 | 4 | 96445632 | C | T |
| 2754 | UNC5C | rs1483735 | 4 | 96454544 | G | A |
| 2755 | UNC5C | rs17023485 | 4 | 96454810 | A | G |
| 2756 | UNC5C | rs2220105 | 4 | 96459368 | T | G |
| 2757 | UNC5C | rs2170780 | 4 | 96463264 | A | G |
| 2758 | UNC5C | rs10002089 | 4 | 96465484 | G | A |
| 2759 | UNC5C | rs17023534 | 4 | 96465529 | T | — |
| 2760 | UNC5C | rs2621462 | 4 | 96466521 | C | T |
| 2761 | UNC5C | rs2626053 | 4 | 96474111 | C | T |
| 2762 | UNC5C | rs10516965 | 4 | 96474837 | T | C |
| 2763 | UNC5C | rs1384868 | 4 | 96475228 | G | T |
| 2764 | UNC5C | rs1843018 | 4 | 96475696 | T | C |
| 2765 | UNC5C | rs1552225 | 4 | 96479865 | A | G |
| 2766 | UNC5C | rs7668316 | 4 | 96485046 | A | G |
| 2767 | UNC5C | rs2626074 | 4 | 96498747 | C | A |
| 2768 | UNC5C | rs6532552 | 4 | 96502034 | T | C |
| 2769 | UNC5C | rs2626035 | 4 | 96505816 | G | A |
| 2770 | UNC5C | rs6846874 | 4 | 96512645 | A | G |
| 2771 | UNC5C | rs2621456 | 4 | 96517598 | A | G |
| 2772 | UNC5C | rs7441708 | 4 | 96519192 | G | A |
| 2773 | UNC5C | rs10222858 | 4 | 96519850 | T | C |
| 2774 | UNC5C | rs12507717 | 4 | 96522308 | T | C |
| 2775 | UNC5C | rs10461127 | 4 | 96523031 | C | T |
| 2776 | UNC5C | rs7674643 | 4 | 96523191 | T | C |
| 2777 | UNC5C | rs17437679 | 4 | 96524607 | C | A |
| 2778 | UNC5C | rs6532555 | 4 | 96526863 | C | T |
| 2779 | UNC5C | rs1384862 | 4 | 96528234 | T | C |
| 2780 | UNC5C | rs10516969 | 4 | 96529186 | G | A |
| 2781 | UNC5C | rs10516970 | 4 | 96529259 | C | T |
| 2782 | UNC5C | rs10440376 | 4 | 96530280 | A | G |
| 2783 | UNC5C | rs1351770 | 4 | 96530820 | G | A |
| 2784 | UNC5C | rs1483739 | 4 | 96533283 | G | T |
| 2785 | UNC5C | rs10012134 | 4 | 96533879 | G | A |
| 2786 | UNC5C | rs7679033 | 4 | 96533897 | C | T |
| 2787 | UNC5C | rs6854948 | 4 | 96534652 | A | G |
| 2788 | UNC5C | rs6832475 | 4 | 96534729 | T | G |
| 2789 | UNC5C | rs9998674 | 4 | 96535055 | G | T |
| 2790 | UNC5C | rs11943185 | 4 | 96535792 | A | G |
| 2791 | UNC5C | rs4406027 | 4 | 96541827 | T | C |
| 2792 | UNC5C | rs7691901 | 4 | 96549440 | T | G |
| 2793 | UNC5C | rs7692318 | 4 | 96549668 | T | C |
| 2794 | UNC5C | rs17023779 | 4 | 96552060 | T | G |
| 2795 | UNC5C | rs17439081 | 4 | 96554513 | C | T |
| 2796 | UNC5C | rs1384863 | 4 | 96557856 | G | A |
| 2797 | UNC5C | rs10516971 | 4 | 96560069 | A | G |
| 2798 | UNC5C | rs9307159 | 4 | 96560909 | C | T |
| 2799 | UNC5C | rs6816601 | 4 | 96561260 | A | C |
| 2800 | UNC5C | rs900617 | 4 | 96563290 | G | A |
| 2801 | UNC5C | rs4380535 | 4 | 96568430 | G | A |
| 2802 | UNC5C | rs1483749 | 4 | 96568443 | G | A |
| 2803 | UNC5C | rs1483748 | 4 | 96572196 | C | T |
| 2804 | UNC5C | rs1384866 | 4 | 96573344 | A | G |
| 2805 | UNC5C | rs13116794 | 4 | 96576950 | T | C |
| 2806 | UNC5C | rs3846457 | 4 | 96579665 | T | C |
| 2807 | UNC5C | rs3846456 | 4 | 96579809 | A | G |
| 2808 | UNC5C | rs265015 | 4 | 96579819 | G | A |
| 2809 | UNC5C | rs3912477 | 4 | 96580176 | T | C |
| 2810 | UNC5C | rs17023881 | 4 | 96581206 | C | T |
| 2811 | UNC5C | rs265018 | 4 | 96581520 | A | G |
| 2812 | UNC5C | rs4699266 | 4 | 96582932 | G | A |
| 2813 | UNC5C | rs17381177 | 4 | 96584243 | T | C |
| 2814 | UNC5C | rs9307160 | 4 | 96586977 | T | C |
| 2815 | UNC5C | rs265063 | 4 | 96587445 | T | C |
| 2816 | UNC5C | rs265062 | 4 | 96588693 | C | T |
| 2817 | UNC5C | rs9995935 | 4 | 96588759 | G | A |
| 2818 | UNC5C | rs174700 | 4 | 96589258 | G | A |
| 2819 | UNC5C | rs10014446 | 4 | 96591138 | T | G |
| 2820 | UNC5C | rs265052 | 4 | 96591634 | C | T |
| 2821 | UNC5C | rs265046 | 4 | 96592873 | G | A |
| 2822 | UNC5C | rs265045 | 4 | 96593099 | G | T |
| 2823 | UNC5C | rs265044 | 4 | 96593143 | T | C |
| 2824 | UNC5C | rs13119662 | 4 | 96593295 | T | C |
| 2825 | UNC5C | rs265043 | 4 | 96594189 | G | A |
| 2826 | UNC5C | rs17023932 | 4 | 96594565 | T | C |
| 2827 | UNC5C | rs265042 | 4 | 96594841 | G | A |
| 2828 | UNC5C | rs11730759 | 4 | 96596607 | T | C |
| 2829 | UNC5C | rs17023940 | 4 | 96596617 | C | A |
| 2830 | UNC5C | rs7669439 | 4 | 96597507 | C | T |
| 2831 | UNC5C | rs17023944 | 4 | 96598204 | C | T |
| 2832 | UNC5C | rs265039 | 4 | 96598249 | T | C |
| 2833 | UNC5C | rs3843446 | 4 | 96599296 | T | C |
| 2834 | UNC5C | rs3846452 | 4 | 96602279 | C | T |
| 2835 | UNC5C | rs4444836 | 4 | 96607674 | T | C |
| 2836 | UNC5C | rs10856918 | 4 | 96610041 | T | G |
| 2837 | UNC5C | rs6832346 | 4 | 96611274 | C | T |
| 2838 | UNC5C | rs10013079 | 4 | 96612140 | A | G |
| 2839 | UNC5C | rs265060 | 4 | 96612588 | T | C |
| 2840 | UNC5C | rs2903010 | 4 | 96619473 | A | G |
| 2841 | UNC5C | rs265070 | 4 | 96620670 | A | G |
| 2842 | UNC5C | rs4505826 | 4 | 96625973 | C | T |
| 2843 | UNC5C | rs6834411 | 4 | 96626795 | A | G |
| 2844 | UNC5C | rs2626047 | 4 | 96626985 | T | C |
| 2845 | UNC5C | rs2629833 | 4 | 96628223 | T | C |
| 2846 | UNC5C | rs6532561 | 4 | 96635495 | C | T |
| 2847 | UNC5C | rs10003330 | 4 | 96643946 | A | G |
| 2848 | UNC5C | rs17384467 | 4 | 96645371 | T | C |
| 2849 | UNC5C | rs17024100 | 4 | 96647732 | C | — |
| 2850 | UNC5C | rs7699716 | 4 | 96648015 | A | G |
| 2851 | UNC5C | rs2865467 | 4 | 96657497 | T | C |
| 2852 | UNC5C | rs11934067 | 4 | 96660579 | C | A |
| 2853 | UNC5C | rs1564896 | 4 | 96667710 | A | G |
| 2854 | UNC5C | rs10021966 | 4 | 96669116 | T | C |
| 2855 | UNC5C | rs17386091 | 4 | 96675573 | C | T |
| 2856 | UNC5C | rs1385669 | 4 | 96677875 | G | A |
| 2857 | UNC5C | rs10516978 | 4 | 96680437 | G | A |
| 2858 | UNC5C | rs7686209 | 4 | 96683220 | G | A |
| 2859 | UNC5C | rs1564895 | 4 | 96686400 | G | A |
| 2860 | UNC5C | rs13149449 | 4 | 96687183 | G | A |
| 2861 | UNC5C | rs998065 | 4 | 96687409 | A | G |
| 2862 | PPP3CA | rs10000538 | 4 | 102161506 | T | C |
| 2863 | PPP3CA | rs3804350 | 4 | 102166953 | C | T |
| 2864 | PPP3CA | rs1880942 | 4 | 102172205 | A | G |
| 2865 | PPP3CA | rs2037773 | 4 | 102177640 | T | C |
| 2866 | PPP3CA | rs2251238 | 4 | 102180970 | C | T |
| 2867 | PPP3CA | rs10025768 | 4 | 102184318 | G | A |
| 2868 | PPP3CA | rs2851062 | 4 | 102187634 | A | G |
| 2869 | PPP3CA | rs1358312 | 4 | 102190474 | G | A |
| 2870 | PPP3CA | rs10026659 | 4 | 102194878 | G | A |
| 2871 | PPP3CA | rs6851231 | 4 | 102202630 | T | G |
| 2872 | PPP3CA | rs10020845 | 4 | 102208603 | T | C |
| 2873 | PPP3CA | rs10516474 | 4 | 102208839 | G | A |
| 2874 | PPP3CA | rs2695203 | 4 | 102214410 | C | T |
| 2875 | PPP3CA | rs3804357 | 4 | 102221146 | C | A |
| 2876 | PPP3CA | rs9307252 | 4 | 102221389 | C | T |
| 2877 | PPP3CA | rs2732504 | 4 | 102228850 | A | G |
| 2878 | PPP3CA | rs17030746 | 4 | 102235509 | C | A |
| 2879 | PPP3CA | rs9990461 | 4 | 102238088 | C | T |
| 2880 | PPP3CA | rs9990811 | 4 | 102238274 | G | T |
| 2881 | PPP3CA | rs17030758 | 4 | 102245160 | A | G |
| 2882 | PPP3CA | rs1876267 | 4 | 102248524 | G | T |
| 2883 | PPP3CA | rs2732510 | 4 | 102250798 | A | C |
| 2884 | PPP3CA | rs10516473 | 4 | 102251531 | C | A |
| 2885 | PPP3CA | rs2659546 | 4 | 102256742 | G | A |
| 2886 | PPP3CA | rs12639641 | 4 | 102259114 | T | C |
| 2887 | PPP3CA | rs6820738 | 4 | 102260259 | G | T |
| 2888 | PPP3CA | rs17829566 | 4 | 102261072 | C | T |
| 2889 | PPP3CA | rs1506801 | 4 | 102262633 | G | A |
| 2890 | PPP3CA | rs17030795 | 4 | 102267099 | A | G |
| 2891 | PPP3CA | rs2732514 | 4 | 102277865 | G | A |
| 2892 | PPP3CA | rs963065 | 4 | 102284654 | G | A |
| 2893 | PPP3CA | rs1405688 | 4 | 102293157 | T | C |
| 2894 | PPP3CA | rs2695219 | 4 | 102302915 | A | C |
| 2895 | PPP3CA | rs4478191 | 4 | 102303778 | C | T |
| 2896 | PPP3CA | rs13142024 | 4 | 102308872 | G | A |
| 2897 | PPP3CA | rs2659501 | 4 | 102312830 | T | C |
| 2898 | PPP3CA | rs4699110 | 4 | 102313476 | T | G |
| 2899 | PPP3CA | rs2659504 | 4 | 102314520 | T | C |
| 2900 | PPP3CA | rs2850982 | 4 | 102319394 | T | C |
| 2901 | PPP3CA | rs1848116 | 4 | 102322963 | T | C |
| 2902 | PPP3CA | rs2659506 | 4 | 102328087 | C | T |
| 2903 | PPP3CA | rs3811063 | 4 | 102339313 | G | A |
| 2904 | PPP3CA | rs2850992 | 4 | 102341778 | T | C |
| 2905 | PPP3CA | rs2659533 | 4 | 102348553 | C | T |
| 2906 | PPP3CA | rs1157038 | 4 | 102359280 | C | A |
| 2907 | PPP3CA | rs17030959 | 4 | 102372254 | C | T |
| 2908 | PPP3CA | rs978373 | 4 | 102373367 | C | T |
| 2909 | PPP3CA | rs2850950 | 4 | 102377473 | G | A |
| 2910 | PPP3CA | rs17030994 | 4 | 102384940 | C | — |
| 2911 | PPP3CA | rs2850373 | 4 | 102387257 | G | A |
| 2912 | PPP3CA | rs2850965 | 4 | 102397986 | T | G |
| 2913 | PPP3CA | rs2850971 | 4 | 102403649 | C | T |
| 2914 | PPP3CA | rs2850343 | 4 | 102416878 | C | T |
| 2915 | PPP3CA | rs2583401 | 4 | 102423552 | G | A |
| 2916 | PPP3CA | rs2850350 | 4 | 102424408 | C | T |
| 2917 | PPP3CA | rs11943655 | 4 | 102430147 | C | — |
| 2918 | PPP3CA | rs6852347 | 4 | 102436590 | T | C |
| 2919 | PPP3CA | rs1348161 | 4 | 102471719 | G | A |
| 2920 | PPP3CA | rs6842191 | 4 | 102477407 | T | — |
| 2921 | PPP3CA | rs10516469 | 4 | 102480276 | G | A |
| 2922 | PPP3CA | rs17031191 | 4 | 102480291 | C | T |
| 2923 | SEMA5A | rs786840 | 5 | 9094935 | T | C |
| 2924 | SEMA5A | rs2290735 | 5 | 9097411 | T | C |
| 2925 | SEMA5A | rs11742324 | 5 | 9112996 | A | G |
| 2926 | SEMA5A | rs786847 | 5 | 9119464 | C | T |
| 2927 | SEMA5A | rs4701834 | 5 | 9123589 | T | C |
| 2928 | SEMA5A | rs2892421 | 5 | 9129917 | A | G |
| 2929 | SEMA5A | rs16881928 | 5 | 9131372 | G | A |
| 2930 | SEMA5A | rs1505062 | 5 | 9135481 | G | A |
| 2931 | SEMA5A | rs1013720 | 5 | 9140921 | T | C |
| 2932 | SEMA5A | rs253634 | 5 | 9144164 | G | A |
| 2933 | SEMA5A | rs17309469 | 5 | 9153211 | G | A |
| 2934 | SEMA5A | rs2456237 | 5 | 9158283 | T | C |
| 2935 | SEMA5A | rs7731508 | 5 | 9160887 | T | C |
| 2936 | SEMA5A | rs2657031 | 5 | 9165040 | T | C |
| 2937 | SEMA5A | rs190423 | 5 | 9174472 | A | G |
| 2938 | SEMA5A | rs3777253 | 5 | 9174591 | G | T |
| 2939 | SEMA5A | rs2657033 | 5 | 9183044 | A | G |
| 2940 | SEMA5A | rs10513005 | 5 | 9183961 | A | G |
| 2941 | SEMA5A | rs6883525 | 5 | 9189487 | A | C |
| 2942 | SEMA5A | rs10057636 | 5 | 9190550 | C | T |
| 2943 | SEMA5A | rs983856 | 5 | 9193848 | G | A |
| 2944 | SEMA5A | rs12519618 | 5 | 9195719 | T | C |
| 2945 | SEMA5A | rs7723025 | 5 | 9204237 | C | A |
| 2946 | SEMA5A | rs899576 | 5 | 9208420 | C | A |
| 2947 | SEMA5A | rs886509 | 5 | 9209012 | G | A |
| 2948 | SEMA5A | rs1806138 | 5 | 9210454 | C | T |
| 2949 | SEMA5A | rs2526121 | 5 | 9211742 | C | T |
| 2950 | SEMA5A | rs2619915 | 5 | 9214347 | T | C |
| 2951 | SEMA5A | rs3846574 | 5 | 9214412 | C | T |
| 2952 | SEMA5A | rs40721 | 5 | 9221231 | C | T |
| 2953 | SEMA5A | rs3797928 | 5 | 9223323 | C | T |
| 2954 | SEMA5A | rs40720 | 5 | 9223857 | T | C |
| 2955 | SEMA5A | rs40718 | 5 | 9226880 | C | T |
| 2956 | SEMA5A | rs3822773 | 5 | 9227381 | T | G |
| 2957 | SEMA5A | rs10044766 | 5 | 9229431 | C | T |
| 2958 | SEMA5A | rs3777280 | 5 | 9230774 | G | A |
| 2959 | SEMA5A | rs3777281 | 5 | 9230932 | T | C |
| 2960 | SEMA5A | rs13171149 | 5 | 9235717 | T | C |
| 2961 | SEMA5A | rs42353 | 5 | 9240035 | G | A |
| 2962 | SEMA5A | rs40701 | 5 | 9242567 | C | T |
| 2963 | SEMA5A | rs3797937 | 5 | 9244362 | G | A |
| 2964 | SEMA5A | rs1806125 | 5 | 9245881 | T | G |
| 2965 | SEMA5A | rs40693 | 5 | 9247611 | A | G |
| 2966 | SEMA5A | rs40687 | 5 | 9248233 | T | C |
| 2967 | SEMA5A | rs10462863 | 5 | 9249537 | T | C |
| 2968 | SEMA5A | rs1806086 | 5 | 9250548 | T | C |
| 2969 | SEMA5A | rs1806085 | 5 | 9250607 | G | A |
| 2970 | SEMA5A | rs12519261 | 5 | 9251489 | A | G |
| 2971 | SEMA5A | rs10513011 | 5 | 9252652 | G | A |
| 2972 | SEMA5A | rs1806112 | 5 | 9253137 | T | C |
| 2973 | SEMA5A | rs40680 | 5 | 9256989 | T | C |
| 2974 | SEMA5A | rs1805971 | 5 | 9257142 | G | A |
| 2975 | SEMA5A | rs1805964 | 5 | 9262831 | C | A |
| 2976 | SEMA5A | rs10042101 | 5 | 9271895 | C | T |
| 2977 | SEMA5A | rs40669 | 5 | 9275811 | G | A |
| 2978 | SEMA5A | rs17254539 | 5 | 9278088 | C | T |
| 2979 | SEMA5A | rs40666 | 5 | 9281171 | G | T |
| 2980 | SEMA5A | rs7734932 | 5 | 9281481 | C | T |
| 2981 | SEMA5A | rs42495 | 5 | 9284066 | A | C |
| 2982 | SEMA5A | rs185248 | 5 | 9284207 | T | C |
| 2983 | SEMA5A | rs1806011 | 5 | 9287139 | A | G |
| 2984 | SEMA5A | rs1805958 | 5 | 9287541 | C | T |
| 2985 | SEMA5A | rs1806000 | 5 | 9295138 | G | T |
| 2986 | SEMA5A | rs28181 | 5 | 9297526 | C | T |
| 2987 | SEMA5A | rs28180 | 5 | 9297575 | G | T |
| 2988 | SEMA5A | rs12716206 | 5 | 9302231 | C | T |
| 2989 | SEMA5A | rs40713 | 5 | 9307909 | A | G |
| 2990 | SEMA5A | rs1805945 | 5 | 9311084 | A | G |
| 2991 | SEMA5A | rs40708 | 5 | 9317682 | G | A |
| 2992 | SEMA5A | rs1805939 | 5 | 9319755 | T | C |
| 2993 | SEMA5A | rs4702617 | 5 | 9320171 | A | G |
| 2994 | SEMA5A | rs1805918 | 5 | 9321737 | G | A |
| 2995 | SEMA5A | rs3777303 | 5 | 9333338 | A | G |
| 2996 | SEMA5A | rs6873909 | 5 | 9340073 | A | G |
| 2997 | SEMA5A | rs3822787 | 5 | 9346213 | A | G |
| 2998 | SEMA5A | rs3777306 | 5 | 9348681 | C | A |
| 2999 | SEMA5A | rs3777308 | 5 | 9349822 | T | C |
| 3000 | SEMA5A | rs13360783 | 5 | 9352238 | G | A |
| 3001 | SEMA5A | rs12655979 | 5 | 9370252 | T | C |
| 3002 | SEMA5A | rs6864327 | 5 | 9373936 | T | G |
| 3003 | SEMA5A | rs3797980 | 5 | 9381316 | G | A |
| 3004 | SEMA5A | rs916990 | 5 | 9384490 | C | T |
| 3005 | SEMA5A | rs1205731 | 5 | 9388344 | T | C |
| 3006 | SEMA5A | rs1205725 | 5 | 9396913 | C | T |
| 3007 | SEMA5A | rs1205724 | 5 | 9396999 | T | C |
| 3008 | SEMA5A | rs1024489 | 5 | 9409174 | C | T |
| 3009 | SEMA5A | rs1024487 | 5 | 9410007 | G | A |
| 3010 | SEMA5A | rs2463506 | 5 | 9410942 | A | G |
| 3011 | SEMA5A | rs10491235 | 5 | 9411953 | G | — |
| 3012 | SEMA5A | rs3797996 | 5 | 9413641 | C | T |
| 3013 | SEMA5A | rs3798004 | 5 | 9423945 | C | T |
| 3014 | SEMA5A | rs42192 | 5 | 9426448 | A | G |
| 3015 | SEMA5A | rs28263 | 5 | 9426641 | T | G |
| 3016 | SEMA5A | rs1205720 | 5 | 9428383 | T | C |
| 3017 | SEMA5A | rs194261 | 5 | 9429118 | A | G |
| 3018 | SEMA5A | rs805153 | 5 | 9433932 | C | T |
| 3019 | SEMA5A | rs40725 | 5 | 9438813 | G | A |
| 3020 | SEMA5A | rs16882600 | 5 | 9439310 | C | T |
| 3021 | SEMA5A | rs42196 | 5 | 9439591 | G | T |
| 3022 | SEMA5A | rs17256306 | 5 | 9442281 | G | A |
| 3023 | SEMA5A | rs13153629 | 5 | 9442867 | C | T |
| 3024 | SEMA5A | rs390322 | 5 | 9443217 | G | A |
| 3025 | SEMA5A | rs1877459 | 5 | 9444753 | C | T |
| 3026 | SEMA5A | rs415024 | 5 | 9445358 | C | A |
| 3027 | SEMA5A | rs10513007 | 5 | 9448453 | T | C |
| 3028 | SEMA5A | rs268489 | 5 | 9448673 | C | T |
| 3029 | SEMA5A | rs788469 | 5 | 9450850 | C | T |
| 3030 | SEMA5A | rs410741 | 5 | 9454606 | C | A |
| 3031 | SEMA5A | rs12519176 | 5 | 9455977 | C | T |
| 3032 | SEMA5A | rs17323208 | 5 | 9456456 | T | C |
| 3033 | SEMA5A | rs268487 | 5 | 9456974 | T | C |
| 3034 | SEMA5A | rs12659273 | 5 | 9461480 | C | T |
| 3035 | SEMA5A | rs2463503 | 5 | 9462056 | G | A |
| 3036 | SEMA5A | rs6879538 | 5 | 9464249 | C | T |
| 3037 | SEMA5A | rs425620 | 5 | 9466362 | C | T |
| 3038 | SEMA5A | rs448038 | 5 | 9466495 | T | G |
| 3039 | SEMA5A | rs10475467 | 5 | 9468717 | G | A |
| 3040 | SEMA5A | rs666617 | 5 | 9473374 | T | C |
| 3041 | SEMA5A | rs406898 | 5 | 9473645 | A | C |
| 3042 | SEMA5A | rs17258120 | 5 | 9476834 | G | T |
| 3043 | SEMA5A | rs7724626 | 5 | 9477801 | G | T |
| 3044 | SEMA5A | rs268486 | 5 | 9479734 | G | A |
| 3045 | SEMA5A | rs3846587 | 5 | 9482200 | T | C |
| 3046 | SEMA5A | rs11951666 | 5 | 9482290 | T | G |
| 3047 | SEMA5A | rs16882748 | 5 | 9485725 | G | A |
| 3048 | SEMA5A | rs2135074 | 5 | 9486320 | G | A |
| 3049 | SEMA5A | rs3798012 | 5 | 9491493 | G | A |
| 3050 | SEMA5A | rs1858428 | 5 | 9491538 | C | T |
| 3051 | SEMA5A | rs3798014 | 5 | 9491566 | A | C |
| 3052 | SEMA5A | rs268534 | 5 | 9491778 | T | C |
| 3053 | SEMA5A | rs3756725 | 5 | 9492642 | G | A |
| 3054 | SEMA5A | rs268532 | 5 | 9494720 | T | C |
| 3055 | SEMA5A | rs2135072 | 5 | 9495007 | A | G |
| 3056 | SEMA5A | rs3822796 | 5 | 9495029 | C | T |
| 3057 | SEMA5A | rs268529 | 5 | 9495994 | T | G |
| 3058 | SEMA5A | rs1512508 | 5 | 9498461 | G | A |
| 3059 | SEMA5A | rs3798024 | 5 | 9500039 | A | G |
| 3060 | SEMA5A | rs268525 | 5 | 9500501 | G | A |
| 3061 | SEMA5A | rs3798028 | 5 | 9500717 | G | T |
| 3062 | SEMA5A | rs440396 | 5 | 9503621 | G | A |
| 3063 | SEMA5A | rs3822799 | 5 | 9503746 | C | T |
| 3064 | SEMA5A | rs436243 | 5 | 9506089 | T | G |
| 3065 | SEMA5A | rs268542 | 5 | 9509166 | T | G |
| 3066 | SEMA5A | rs3798031 | 5 | 9510243 | C | T |
| 3067 | SEMA5A | rs397287 | 5 | 9516291 | C | A |
| 3068 | SEMA5A | rs13166820 | 5 | 9521015 | C | T |
| 3069 | SEMA5A | rs12657858 | 5 | 9521865 | G | A |
| 3070 | SEMA5A | rs433755 | 5 | 9527946 | G | T |
| 3071 | SEMA5A | rs418768 | 5 | 9535178 | A | G |
| 3072 | SEMA5A | rs418446 | 5 | 9536209 | C | T |
| 3073 | SEMA5A | rs17260496 | 5 | 9538732 | G | A |
| 3074 | SEMA5A | rs950050 | 5 | 9541689 | A | G |
| 3075 | SEMA5A | rs17260912 | 5 | 9546723 | T | C |
| 3076 | SEMA5A | rs12657696 | 5 | 9548607 | T | C |
| 3077 | SEMA5A | rs3777364 | 5 | 9553530 | C | T |
| 3078 | SEMA5A | rs421548 | 5 | 9561979 | C | T |
| 3079 | SEMA5A | rs11959773 | 5 | 9562811 | A | C |
| 3080 | SEMA5A | rs3798063 | 5 | 9572910 | C | T |
| 3081 | SEMA5A | rs3798064 | 5 | 9572978 | A | G |
| 3082 | SEMA5A | rs465107 | 5 | 9575303 | A | G |
| 3083 | SEMA5A | rs3798068 | 5 | 9576375 | C | T |
| 3084 | SEMA5A | rs465667 | 5 | 9580976 | C | T |
| 3085 | SEMA5A | rs465991 | 5 | 9582244 | C | T |
| 3086 | SEMA5A | rs174863 | 5 | 9586705 | G | A |
| 3087 | SEMA5A | rs268550 | 5 | 9586854 | G | A |
| 3088 | SEMA5A | rs268551 | 5 | 9587206 | G | T |
| 3089 | SEMA5A | rs6881628 | 5 | 9592540 | T | G |
| 3090 | SEMA5A | rs605126 | 5 | 9594866 | T | C |
| 3091 | SEMA5A | rs3798097 | 5 | 9595529 | C | T |
| 3092 | SEMA5A | rs17329240 | 5 | 9600305 | C | T |
| 3093 | SEMA5A | rs2271452 | 5 | 9600708 | G | A |
| 3094 | SEMA5A | rs1398337 | 5 | 9601098 | C | T |
| 3095 | SEMA5A | rs17329485 | 5 | 9602386 | C | T |
| 3096 | SEMA5A | rs930076 | 5 | 9603642 | A | G |
| 3097 | SEMA5A | rs16883128 | 5 | 9603770 | G | A |
| 3098 | SEMA5A | rs17329702 | 5 | 9604091 | C | T |
| 3099 | RASA1 | rs1567348 | 5 | 86621630 | G | A |
| 3100 | RASA1 | rs6893772 | 5 | 86632987 | G | A |
| 3101 | RASA1 | rs12659717 | 5 | 86635048 | T | C |
| 3102 | RASA1 | rs6861271 | 5 | 86635968 | T | G |
| 3103 | RASA1 | rs13362486 | 5 | 86639581 | C | A |
| 3104 | RASA1 | rs3752862 | 5 | 86669729 | G | A |
| 3105 | RASA1 | rs16902633 | 5 | 86692624 | C | T |
| 3106 | RASA1 | rs10067098 | 5 | 86706726 | T | C |
| 3107 | RASA1 | rs3827607 | 5 | 86712976 | G | A |
| 3108 | EFNA5 | rs365807 | 5 | 106741517 | T | C |
| 3109 | EFNA5 | rs252815 | 5 | 106749582 | T | C |
| 3110 | EFNA5 | rs252818 | 5 | 106753590 | T | C |
| 3111 | EFNA5 | rs17412225 | 5 | 106756485 | T | C |
| 3112 | EFNA5 | rs252813 | 5 | 106761925 | A | G |
| 3113 | EFNA5 | rs6868410 | 5 | 106777589 | A | G |
| 3114 | EFNA5 | rs185221 | 5 | 106781604 | G | A |
| 3115 | EFNA5 | rs2731840 | 5 | 106789035 | T | C |
| 3116 | EFNA5 | rs252802 | 5 | 106790483 | T | C |
| 3117 | EFNA5 | rs17533273 | 5 | 106791538 | A | G |
| 3118 | EFNA5 | rs187557 | 5 | 106794239 | T | C |
| 3119 | EFNA5 | rs252991 | 5 | 106795245 | G | A |
| 3120 | EFNA5 | rs3843490 | 5 | 106795421 | T | C |
| 3121 | EFNA5 | rs17448944 | 5 | 106797022 | C | T |
| 3122 | EFNA5 | rs152608 | 5 | 106799108 | G | A |
| 3123 | EFNA5 | rs153114 | 5 | 106801094 | A | G |
| 3124 | EFNA5 | rs152600 | 5 | 106805393 | T | G |
| 3125 | EFNA5 | rs152599 | 5 | 106807204 | G | T |
| 3126 | EFNA5 | rs17533525 | 5 | 106807591 | C | T |
| 3127 | EFNA5 | rs152595 | 5 | 106809275 | G | A |
| 3128 | EFNA5 | rs1835111 | 5 | 106817663 | T | C |
| 3129 | EFNA5 | rs537500 | 5 | 106821490 | C | T |
| 3130 | EFNA5 | rs3909464 | 5 | 106821672 | A | C |
| 3131 | EFNA5 | rs152580 | 5 | 106823761 | G | A |
| 3132 | EFNA5 | rs10491390 | 5 | 106828047 | G | A |
| 3133 | EFNA5 | rs17449298 | 5 | 106828481 | C | T |
| 3134 | EFNA5 | rs151957 | 5 | 106831817 | T | C |
| 3135 | EFNA5 | rs26247 | 5 | 106832262 | T | C |
| 3136 | EFNA5 | rs3890134 | 5 | 106832827 | G | A |
| 3137 | EFNA5 | rs2288385 | 5 | 106833950 | A | C |
| 3138 | EFNA5 | rs636137 | 5 | 106834123 | C | T |
| 3139 | EFNA5 | rs29543 | 5 | 106834337 | T | C |
| 3140 | EFNA5 | rs26242 | 5 | 106842541 | C | T |
| 3141 | EFNA5 | rs25997 | 5 | 106847305 | A | G |
| 3142 | EFNA5 | rs25970 | 5 | 106854312 | C | T |
| 3143 | EFNA5 | rs17534089 | 5 | 106857118 | A | G |
| 3144 | EFNA5 | rs31717 | 5 | 106857438 | A | C |
| 3145 | EFNA5 | rs27681 | 5 | 106859473 | G | A |
| 3146 | EFNA5 | rs12520677 | 5 | 106861050 | G | A |
| 3147 | EFNA5 | rs1196516 | 5 | 106862466 | G | A |
| 3148 | EFNA5 | rs25976 | 5 | 106862909 | C | T |
| 3149 | EFNA5 | rs10491393 | 5 | 106864673 | A | G |
| 3150 | EFNA5 | rs26249 | 5 | 106866306 | A | G |
| 3151 | EFNA5 | rs151943 | 5 | 106870127 | A | G |
| 3152 | EFNA5 | rs10491394 | 5 | 106872850 | T | C |
| 3153 | EFNA5 | rs6894788 | 5 | 106875173 | A | G |
| 3154 | EFNA5 | rs152558 | 5 | 106875561 | G | A |
| 3155 | EFNA5 | rs153118 | 5 | 106877354 | A | G |
| 3156 | EFNA5 | rs152555 | 5 | 106877573 | T | C |
| 3157 | EFNA5 | rs250253 | 5 | 106880865 | C | T |
| 3158 | EFNA5 | rs17534319 | 5 | 106882151 | T | C |
| 3159 | EFNA5 | rs395561 | 5 | 106889874 | T | G |
| 3160 | EFNA5 | rs464250 | 5 | 106891614 | A | C |
| 3161 | EFNA5 | rs462885 | 5 | 106891686 | G | A |
| 3162 | EFNA5 | rs153190 | 5 | 106901585 | G | — |
| 3163 | EFNA5 | rs6884705 | 5 | 106906963 | C | T |
| 3164 | EFNA5 | rs662627 | 5 | 106909232 | C | T |
| 3165 | EFNA5 | rs849520 | 5 | 106909446 | C | T |
| 3166 | EFNA5 | rs1966892 | 5 | 106911882 | C | T |
| 3167 | EFNA5 | rs467089 | 5 | 106913813 | A | G |
| 3168 | EFNA5 | rs341272 | 5 | 106914967 | A | G |
| 3169 | EFNA5 | rs352603 | 5 | 106921902 | G | A |
| 3170 | EFNA5 | rs677797 | 5 | 106924383 | G | A |
| 3171 | EFNA5 | rs2484103 | 5 | 106924516 | T | C |
| 3172 | EFNA5 | rs164699 | 5 | 106925876 | T | C |
| 3173 | EFNA5 | rs164849 | 5 | 106928634 | T | C |
| 3174 | EFNA5 | rs12519552 | 5 | 106938809 | A | G |
| 3175 | EFNA5 | rs597083 | 5 | 106941488 | G | A |
| 3176 | EFNA5 | rs641406 | 5 | 106941926 | A | G |
| 3177 | EFNA5 | rs103840 | 5 | 106942632 | A | G |
| 3178 | EFNA5 | rs4957704 | 5 | 106946011 | A | G |
| 3179 | EFNA5 | rs9328005 | 5 | 106950088 | T | — |
| 3180 | EFNA5 | rs180791 | 5 | 106951468 | G | A |
| 3181 | EFNA5 | rs9328010 | 5 | 106952728 | A | G |
| 3182 | EFNA5 | rs3864269 | 5 | 106964864 | G | A |
| 3183 | EFNA5 | rs2112912 | 5 | 106968418 | T | C |
| 3184 | EFNA5 | rs12519591 | 5 | 106971910 | C | A |
| 3185 | EFNA5 | rs26724 | 5 | 106972172 | C | T |
| 3186 | EFNA5 | rs26728 | 5 | 106973955 | C | T |
| 3187 | EFNA5 | rs17160255 | 5 | 106979256 | G | A |
| 3188 | EFNA5 | rs4090594 | 5 | 106984342 | T | C |
| 3189 | EFNA5 | rs164691 | 5 | 106986857 | C | T |
| 3190 | EFNA5 | rs164694 | 5 | 106988723 | G | A |
| 3191 | EFNA5 | rs164840 | 5 | 106990248 | A | C |
| 3192 | EFNA5 | rs352602 | 5 | 106994231 | T | C |
| 3193 | EFNA5 | rs27550 | 5 | 106999467 | C | A |
| 3194 | EFNA5 | rs149382 | 5 | 107008734 | A | G |
| 3195 | EFNA5 | rs10074258 | 5 | 107010459 | C | T |
| 3196 | EFNA5 | rs26731 | 5 | 107012067 | T | C |
| 3197 | EFNA5 | rs17160324 | 5 | 107012707 | G | A |
| 3198 | EFNA5 | rs7732162 | 5 | 107018930 | C | T |
| 3199 | EFNA5 | rs1490827 | 5 | 107038100 | C | T |
| 3200 | EFNA5 | rs6887589 | 5 | 107039458 | A | G |
| 3201 | SEMA6A | rs26591 | 5 | 115803210 | G | A |
| 3202 | SEMA6A | rs26592 | 5 | 115803441 | A | G |
| 3203 | SEMA6A | rs3733724 | 5 | 115807872 | C | T |
| 3204 | SEMA6A | rs2303752 | 5 | 115813411 | C | T |
| 3205 | SEMA6A | rs10447236 | 5 | 115815122 | G | T |
| 3206 | SEMA6A | rs26602 | 5 | 115816003 | G | A |
| 3207 | SEMA6A | rs1963858 | 5 | 115817498 | A | G |
| 3208 | SEMA6A | rs3733725 | 5 | 115819568 | G | A |
| 3209 | SEMA6A | rs34971 | 5 | 115823087 | T | G |
| 3210 | SEMA6A | rs187710 | 5 | 115824361 | A | G |
| 3211 | SEMA6A | rs10077720 | 5 | 115828458 | G | A |
| 3212 | SEMA6A | rs27644 | 5 | 115829601 | C | T |
| 3213 | SEMA6A | rs258019 | 5 | 115835587 | A | G |
| 3214 | SEMA6A | rs2161227 | 5 | 115838042 | G | T |
| 3215 | SEMA6A | rs423834 | 5 | 115839119 | A | G |
| 3216 | SEMA6A | rs34967 | 5 | 115842005 | C | T |
| 3217 | SEMA6A | rs469035 | 5 | 115843506 | G | T |
| 3218 | SEMA6A | rs2278264 | 5 | 115844722 | G | A |
| 3219 | SEMA6A | rs10519470 | 5 | 115847034 | C | T |
| 3220 | SEMA6A | rs17139903 | 5 | 115849114 | C | T |
| 3221 | SEMA6A | rs2059874 | 5 | 115850291 | G | A |
| 3222 | SEMA6A | rs17139912 | 5 | 115850741 | T | C |
| 3223 | SEMA6A | rs9885339 | 5 | 115853167 | C | A |
| 3224 | SEMA6A | rs11956022 | 5 | 115854198 | C | A |
| 3225 | SEMA6A | rs154599 | 5 | 115855255 | A | G |
| 3226 | SEMA6A | rs154601 | 5 | 115855844 | A | C |
| 3227 | SEMA6A | rs1014643 | 5 | 115858368 | A | G |
| 3228 | SEMA6A | rs32972 | 5 | 115858991 | G | A |
| 3229 | SEMA6A | rs2287686 | 5 | 115859229 | T | C |
| 3230 | SEMA6A | rs32974 | 5 | 115859772 | G | A |
| 3231 | SEMA6A | rs258014 | 5 | 115861543 | G | T |
| 3232 | SEMA6A | rs2252892 | 5 | 115861752 | G | A |
| 3233 | SEMA6A | rs10519473 | 5 | 115863270 | A | G |
| 3234 | SEMA6A | rs6885854 | 5 | 115865688 | G | T |
| 3235 | SEMA6A | rs153643 | 5 | 115868106 | A | G |
| 3236 | SEMA6A | rs154575 | 5 | 115868177 | T | G |
| 3237 | SEMA6A | rs153644 | 5 | 115868427 | G | A |
| 3238 | SEMA6A | rs153600 | 5 | 115872595 | A | G |
| 3239 | SEMA6A | rs7701234 | 5 | 115873150 | G | T |
| 3240 | SEMA6A | rs7721479 | 5 | 115873484 | T | C |
| 3241 | SEMA6A | rs918425 | 5 | 115873710 | A | C |
| 3242 | SEMA6A | rs4920916 | 5 | 115875140 | A | G |
| 3243 | SEMA6A | rs6897947 | 5 | 115875945 | T | C |
| 3244 | SEMA6A | rs7728185 | 5 | 115878297 | G | A |
| 3245 | SEMA6A | rs7734164 | 5 | 115879810 | C | T |
| 3246 | SEMA6A | rs6860340 | 5 | 115880859 | A | G |
| 3247 | SEMA6A | rs154577 | 5 | 115885390 | G | T |
| 3248 | SEMA6A | rs7702266 | 5 | 115888956 | T | C |
| 3249 | SEMA6A | rs154576 | 5 | 115889531 | T | C |
| 3250 | SEMA6A | rs153645 | 5 | 115892477 | T | C |
| 3251 | SEMA6A | rs7734835 | 5 | 115897159 | C | T |
| 3252 | SEMA6A | rs17140058 | 5 | 115902413 | A | — |
| 3253 | SEMA6A | rs10077439 | 5 | 115909328 | T | C |
| 3254 | SEMA6A | rs254228 | 5 | 115913499 | T | C |
| 3255 | SEMA6A | rs254229 | 5 | 115913571 | T | C |
| 3256 | SEMA6A | rs254230 | 5 | 115913757 | A | G |
| 3257 | SEMA6A | rs254234 | 5 | 115916094 | C | T |
| 3258 | SEMA6A | rs153641 | 5 | 115919688 | G | A |
| 3259 | SEMA6A | rs10055012 | 5 | 115928562 | T | G |
| 3260 | SEMA6A | rs2840113 | 5 | 115931808 | A | G |
| 3261 | ABLIM3 | rs4147470 | 5 | 148508300 | T | C |
| 3262 | ABLIM3 | rs4546368 | 5 | 148519293 | T | C |
| 3263 | ABLIM3 | rs4361503 | 5 | 148525002 | C | T |
| 3264 | ABLIM3 | rs9325132 | 5 | 148528200 | C | T |
| 3265 | ABLIM3 | rs9765483 | 5 | 148528716 | G | A |
| 3266 | ABLIM3 | rs6881442 | 5 | 148529304 | G | A |
| 3267 | ABLIM3 | rs9325134 | 5 | 148531358 | A | G |
| 3268 | ABLIM3 | rs17109726 | 5 | 148540080 | T | — |
| 3269 | ABLIM3 | rs4362930 | 5 | 148540102 | T | C |
| 3270 | ABLIM3 | rs4321739 | 5 | 148552818 | T | C |
| 3271 | ABLIM3 | rs12108753 | 5 | 148552843 | G | — |
| 3272 | ABLIM3 | rs4277904 | 5 | 148559330 | A | G |
| 3273 | ABLIM3 | rs10463421 | 5 | 148561651 | C | A |
| 3274 | ABLIM3 | rs13167773 | 5 | 148567182 | C | T |
| 3275 | ABLIM3 | rs2918285 | 5 | 148567746 | A | G |
| 3276 | ABLIM3 | rs13174812 | 5 | 148573665 | G | A |
| 3277 | ABLIM3 | rs2963495 | 5 | 148576568 | T | G |
| 3278 | ABLIM3 | rs10065552 | 5 | 148582009 | C | T |
| 3279 | ABLIM3 | rs2918268 | 5 | 148586949 | C | T |
| 3280 | ABLIM3 | rs1465850 | 5 | 148591400 | G | T |
| 3281 | ABLIM3 | rs13161083 | 5 | 148596244 | G | T |
| 3282 | ABLIM3 | rs748066 | 5 | 148600728 | T | C |
| 3283 | ABLIM3 | rs10073851 | 5 | 148603410 | A | G |
| 3284 | ABLIM3 | rs3816159 | 5 | 148605091 | A | G |
| 3285 | ABLIM3 | rs2289285 | 5 | 148611445 | C | T |
| 3286 | ABLIM3 | rs1438689 | 5 | 148611626 | T | C |
| 3287 | ABLIM3 | rs2018446 | 5 | 148613937 | C | A |
| 3288 | ABLIM3 | rs6887404 | 5 | 148622110 | A | C |
| 3289 | SLIT3 | rs1048307 | 5 | 168026100 | C | T |
| 3290 | SLIT3 | rs12655287 | 5 | 168032010 | G | A |
| 3291 | SLIT3 | rs1870563 | 5 | 168036018 | A | C |
| 3292 | SLIT3 | rs12521041 | 5 | 168041434 | C | T |
| 3293 | SLIT3 | rs292488 | 5 | 168041837 | G | A |
| 3294 | SLIT3 | rs744544 | 5 | 168048310 | A | G |
| 3295 | SLIT3 | rs17634452 | 5 | 168049371 | G | T |
| 3296 | SLIT3 | rs17665158 | 5 | 168050686 | T | C |
| 3297 | SLIT3 | rs6869359 | 5 | 168057577 | C | T |
| 3298 | SLIT3 | rs4868489 | 5 | 168066407 | A | G |
| 3299 | SLIT3 | rs1864951 | 5 | 168071847 | C | T |
| 3300 | SLIT3 | rs11746295 | 5 | 168082337 | A | G |
| 3301 | SLIT3 | rs6890724 | 5 | 168096655 | G | A |
| 3302 | SLIT3 | rs10516049 | 5 | 168097247 | A | G |
| 3303 | SLIT3 | rs11743823 | 5 | 168097322 | C | T |
| 3304 | SLIT3 | rs10072233 | 5 | 168097883 | T | C |
| 3305 | SLIT3 | rs2337552 | 5 | 168099870 | C | T |
| 3306 | SLIT3 | rs7728435 | 5 | 168099959 | T | C |
| 3307 | SLIT3 | rs4502845 | 5 | 168107618 | T | G |
| 3308 | SLIT3 | rs3822657 | 5 | 168107869 | T | C |
| 3309 | SLIT3 | rs2304035 | 5 | 168109095 | A | G |
| 3310 | SLIT3 | rs11746188 | 5 | 168112127 | A | G |
| 3311 | SLIT3 | rs10036727 | 5 | 168112659 | T | C |
| 3312 | SLIT3 | rs1549910 | 5 | 168113167 | A | G |
| 3313 | SLIT3 | rs2288790 | 5 | 168113201 | C | T |
| 3314 | SLIT3 | rs1549909 | 5 | 168113248 | A | G |
| 3315 | SLIT3 | rs7718883 | 5 | 168115570 | C | T |
| 3316 | SLIT3 | rs6862713 | 5 | 168116314 | A | G |
| 3317 | SLIT3 | rs11745138 | 5 | 168116687 | A | G |
| 3318 | SLIT3 | rs11742226 | 5 | 168120249 | G | A |
| 3319 | SLIT3 | rs7725943 | 5 | 168120736 | C | T |
| 3320 | SLIT3 | rs17070430 | 5 | 168121258 | G | A |
| 3321 | SLIT3 | rs11745421 | 5 | 168123644 | T | C |
| 3322 | SLIT3 | rs1030661 | 5 | 168124024 | A | G |
| 3323 | SLIT3 | rs17732939 | 5 | 168124390 | C | T |
| 3324 | SLIT3 | rs11743764 | 5 | 168125453 | G | A |
| 3325 | SLIT3 | rs1014194 | 5 | 168125522 | T | G |
| 3326 | SLIT3 | rs17070438 | 5 | 168125772 | A | G |
| 3327 | SLIT3 | rs6860854 | 5 | 168126754 | T | C |
| 3328 | SLIT3 | rs9784690 | 5 | 168128612 | C | T |
| 3329 | SLIT3 | rs6555826 | 5 | 168130042 | A | C |
| 3330 | SLIT3 | rs1875976 | 5 | 168130379 | T | C |
| 3331 | SLIT3 | rs2082400 | 5 | 168130404 | T | C |
| 3332 | SLIT3 | rs868103 | 5 | 168130883 | G | A |
| 3333 | SLIT3 | rs2053046 | 5 | 168131545 | A | G |
| 3334 | SLIT3 | rs10516050 | 5 | 168131879 | C | T |
| 3335 | SLIT3 | rs4867902 | 5 | 168132202 | T | C |
| 3336 | SLIT3 | rs2288794 | 5 | 168132682 | T | C |
| 3337 | SLIT3 | rs4867903 | 5 | 168135219 | C | T |
| 3338 | SLIT3 | rs17665552 | 5 | 168135306 | T | C |
| 3339 | SLIT3 | rs4867905 | 5 | 168135447 | C | T |
| 3340 | SLIT3 | rs17733068 | 5 | 168136033 | A | G |
| 3341 | SLIT3 | rs10866630 | 5 | 168138231 | C | T |
| 3342 | SLIT3 | rs17665652 | 5 | 168140274 | C | T |
| 3343 | SLIT3 | rs871097 | 5 | 168140685 | T | C |
| 3344 | SLIT3 | rs871098 | 5 | 168140959 | A | C |
| 3345 | SLIT3 | rs2163761 | 5 | 168143832 | G | A |
| 3346 | SLIT3 | rs10475891 | 5 | 168144674 | T | C |
| 3347 | SLIT3 | rs11134531 | 5 | 168144746 | A | G |
| 3348 | SLIT3 | rs2292141 | 5 | 168145265 | G | A |
| 3349 | SLIT3 | rs2292143 | 5 | 168145585 | G | A |
| 3350 | SLIT3 | rs4298263 | 5 | 168148428 | A | G |
| 3351 | SLIT3 | rs2247645 | 5 | 168149067 | C | T |
| 3352 | SLIT3 | rs891928 | 5 | 168149345 | G | A |
| 3353 | SLIT3 | rs7728604 | 5 | 168152680 | C | T |
| 3354 | SLIT3 | rs4867939 | 5 | 168156259 | C | T |
| 3355 | SLIT3 | rs17665720 | 5 | 168157173 | A | G |
| 3356 | SLIT3 | rs11134532 | 5 | 168158416 | T | C |
| 3357 | SLIT3 | rs17070500 | 5 | 168159270 | T | C |
| 3358 | SLIT3 | rs11747556 | 5 | 168159408 | G | A |
| 3359 | SLIT3 | rs12659534 | 5 | 168162396 | C | T |
| 3360 | SLIT3 | rs17553805 | 5 | 168163498 | G | T |
| 3361 | SLIT3 | rs10071604 | 5 | 168164371 | G | A |
| 3362 | SLIT3 | rs17070509 | 5 | 168165335 | C | T |
| 3363 | SLIT3 | rs884787 | 5 | 168167095 | A | G |
| 3364 | SLIT3 | rs883241 | 5 | 168167415 | G | A |
| 3365 | SLIT3 | rs867477 | 5 | 168167569 | G | T |
| 3366 | SLIT3 | rs883242 | 5 | 168167758 | T | G |
| 3367 | SLIT3 | rs9885172 | 5 | 168174541 | T | C |
| 3368 | SLIT3 | rs11951140 | 5 | 168177253 | A | G |
| 3369 | SLIT3 | rs1109105 | 5 | 168177604 | A | G |
| 3370 | SLIT3 | rs17635284 | 5 | 168178679 | A | G |
| 3371 | SLIT3 | rs17665948 | 5 | 168179663 | A | G |
| 3372 | SLIT3 | rs6555831 | 5 | 168180029 | A | G |
| 3373 | SLIT3 | rs11134534 | 5 | 168180553 | C | A |
| 3374 | SLIT3 | rs1875977 | 5 | 168183007 | T | C |
| 3375 | SLIT3 | rs17070531 | 5 | 168184154 | C | A |
| 3376 | SLIT3 | rs12691283 | 5 | 168185214 | G | T |
| 3377 | SLIT3 | rs7726584 | 5 | 168187475 | T | G |
| 3378 | SLIT3 | rs4282339 | 5 | 168188818 | G | A |
| 3379 | SLIT3 | rs13170900 | 5 | 168189928 | G | A |
| 3380 | SLIT3 | rs949780 | 5 | 168190701 | C | T |
| 3381 | SLIT3 | rs1394618 | 5 | 168192794 | C | T |
| 3382 | SLIT3 | rs2974427 | 5 | 168193599 | T | C |
| 3383 | SLIT3 | rs2974428 | 5 | 168197822 | C | T |
| 3384 | SLIT3 | rs1979923 | 5 | 168198469 | T | C |
| 3385 | SLIT3 | rs2938763 | 5 | 168211989 | G | A |
| 3386 | SLIT3 | rs2938766 | 5 | 168212789 | A | G |
| 3387 | SLIT3 | rs2938767 | 5 | 168212967 | A | G |
| 3388 | SLIT3 | rs12516521 | 5 | 168213445 | G | A |
| 3389 | SLIT3 | rs6555833 | 5 | 168215742 | A | C |
| 3390 | SLIT3 | rs6555834 | 5 | 168218794 | T | C |
| 3391 | SLIT3 | rs17635646 | 5 | 168218879 | G | A |
| 3392 | SLIT3 | rs1875972 | 5 | 168221570 | G | A |
| 3393 | SLIT3 | rs1466947 | 5 | 168231236 | A | C |
| 3394 | SLIT3 | rs2938772 | 5 | 168239625 | G | A |
| 3395 | SLIT3 | rs1553536 | 5 | 168242670 | G | T |
| 3396 | SLIT3 | rs4616917 | 5 | 168246539 | C | T |
| 3397 | SLIT3 | rs11134544 | 5 | 168252634 | G | T |
| 3398 | SLIT3 | rs7714853 | 5 | 168255812 | C | T |
| 3399 | SLIT3 | rs4573011 | 5 | 168256825 | T | C |
| 3400 | SLIT3 | rs12519316 | 5 | 168257899 | C | T |
| 3401 | SLIT3 | rs10516053 | 5 | 168263029 | T | C |
| 3402 | SLIT3 | rs10069222 | 5 | 168263916 | G | A |
| 3403 | SLIT3 | rs7709047 | 5 | 168270632 | G | T |
| 3404 | SLIT3 | rs12655322 | 5 | 168276858 | T | C |
| 3405 | SLIT3 | rs891958 | 5 | 168285991 | C | T |
| 3406 | SLIT3 | rs1982062 | 5 | 168288225 | G | A |
| 3407 | SLIT3 | rs6879973 | 5 | 168295768 | C | T |
| 3408 | SLIT3 | rs919753 | 5 | 168296182 | G | A |
| 3409 | SLIT3 | rs6862117 | 5 | 168299443 | G | — |
| 3410 | SLIT3 | rs13166368 | 5 | 168301572 | C | A |
| 3411 | SLIT3 | rs11134553 | 5 | 168305256 | A | C |
| 3412 | SLIT3 | rs10042354 | 5 | 168306549 | A | G |
| 3413 | SLIT3 | rs6555837 | 5 | 168308872 | C | T |
| 3414 | SLIT3 | rs6555838 | 5 | 168314072 | G | A |
| 3415 | SLIT3 | rs12188351 | 5 | 168318667 | G | A |
| 3416 | SLIT3 | rs10054320 | 5 | 168318772 | A | G |
| 3417 | SLIT3 | rs6555842 | 5 | 168320022 | C | T |
| 3418 | SLIT3 | rs1432910 | 5 | 168324135 | A | G |
| 3419 | SLIT3 | rs6885618 | 5 | 168324520 | A | G |
| 3420 | SLIT3 | rs9313435 | 5 | 168327584 | A | G |
| 3421 | SLIT3 | rs9313437 | 5 | 168327770 | C | A |
| 3422 | SLIT3 | rs13165487 | 5 | 168332784 | T | C |
| 3423 | SLIT3 | rs6555845 | 5 | 168344147 | C | T |
| 3424 | SLIT3 | rs6899152 | 5 | 168344418 | G | A |
| 3425 | SLIT3 | rs12189509 | 5 | 168351402 | A | G |
| 3426 | SLIT3 | rs11747642 | 5 | 168356485 | T | C |
| 3427 | SLIT3 | rs10062450 | 5 | 168362984 | A | G |
| 3428 | SLIT3 | rs12152726 | 5 | 168365991 | T | C |
| 3429 | SLIT3 | rs10045549 | 5 | 168368359 | G | A |
| 3430 | SLIT3 | rs2163783 | 5 | 168372054 | G | A |
| 3431 | SLIT3 | rs12515863 | 5 | 168372930 | C | T |
| 3432 | SLIT3 | rs17734396 | 5 | 168374605 | G | A |
| 3433 | SLIT3 | rs11954861 | 5 | 168378067 | T | G |
| 3434 | SLIT3 | rs1432906 | 5 | 168378948 | G | A |
| 3435 | SLIT3 | rs6890327 | 5 | 168385055 | C | A |
| 3436 | SLIT3 | rs1432902 | 5 | 168385765 | G | T |
| 3437 | SLIT3 | rs2879135 | 5 | 168387515 | A | G |
| 3438 | SLIT3 | rs10516055 | 5 | 168391110 | T | — |
| 3439 | SLIT3 | rs1432899 | 5 | 168391547 | C | T |
| 3440 | SLIT3 | rs7447181 | 5 | 168391830 | C | T |
| 3441 | SLIT3 | rs1059160 | 5 | 168397319 | T | C |
| 3442 | SLIT3 | rs2278386 | 5 | 168397804 | G | A |
| 3443 | SLIT3 | rs17666992 | 5 | 168399729 | T | G |
| 3444 | SLIT3 | rs7717558 | 5 | 168402556 | G | T |
| 3445 | SLIT3 | rs6555847 | 5 | 168408533 | G | A |
| 3446 | SLIT3 | rs11743301 | 5 | 168410375 | C | T |
| 3447 | SLIT3 | rs7735291 | 5 | 168416172 | G | A |
| 3448 | SLIT3 | rs13154825 | 5 | 168417072 | A | C |
| 3449 | SLIT3 | rs10516057 | 5 | 168418548 | T | C |
| 3450 | SLIT3 | rs882015 | 5 | 168418871 | G | T |
| 3451 | SLIT3 | rs1469683 | 5 | 168421911 | A | G |
| 3452 | SLIT3 | rs4868223 | 5 | 168423758 | A | G |
| 3453 | SLIT3 | rs12656030 | 5 | 168427316 | C | T |
| 3454 | SLIT3 | rs7701998 | 5 | 168427719 | A | C |
| 3455 | SLIT3 | rs753860 | 5 | 168428668 | A | G |
| 3456 | SLIT3 | rs2338296 | 5 | 168428922 | T | C |
| 3457 | SLIT3 | rs6864266 | 5 | 168431255 | T | C |
| 3458 | SLIT3 | rs10054190 | 5 | 168434029 | A | G |
| 3459 | SLIT3 | rs188432 | 5 | 168434786 | G | T |
| 3460 | SLIT3 | rs188433 | 5 | 168434907 | T | C |
| 3461 | SLIT3 | rs6887116 | 5 | 168437389 | A | C |
| 3462 | SLIT3 | rs295986 | 5 | 168438281 | C | T |
| 3463 | SLIT3 | rs2668040 | 5 | 168441010 | C | T |
| 3464 | SLIT3 | rs17667288 | 5 | 168442227 | A | — |
| 3465 | SLIT3 | rs7735612 | 5 | 168442310 | T | C |
| 3466 | SLIT3 | rs2668063 | 5 | 168443030 | G | T |
| 3467 | SLIT3 | rs296009 | 5 | 168445762 | G | T |
| 3468 | SLIT3 | rs296008 | 5 | 168445867 | T | C |
| 3469 | SLIT3 | rs296007 | 5 | 168446206 | T | C |
| 3470 | SLIT3 | rs296006 | 5 | 168447445 | G | A |
| 3471 | SLIT3 | rs167194 | 5 | 168449600 | T | G |
| 3472 | SLIT3 | rs11744892 | 5 | 168452223 | C | T |
| 3473 | SLIT3 | rs296002 | 5 | 168454446 | G | T |
| 3474 | SLIT3 | rs11134558 | 5 | 168454890 | G | A |
| 3475 | SLIT3 | rs13173842 | 5 | 168457263 | C | T |
| 3476 | SLIT3 | rs10067964 | 5 | 168458268 | T | C |
| 3477 | SLIT3 | rs295999 | 5 | 168458445 | G | A |
| 3478 | SLIT3 | rs6883493 | 5 | 168461609 | T | C |
| 3479 | SLIT3 | rs295990 | 5 | 168462749 | C | A |
| 3480 | SLIT3 | rs295996 | 5 | 168465045 | A | G |
| 3481 | SLIT3 | rs2569114 | 5 | 168471818 | C | T |
| 3482 | SLIT3 | rs12522573 | 5 | 168473131 | C | A |
| 3483 | SLIT3 | rs6863538 | 5 | 168475823 | G | A |
| 3484 | SLIT3 | rs10053670 | 5 | 168476226 | G | A |
| 3485 | SLIT3 | rs2668054 | 5 | 168480557 | G | A |
| 3486 | SLIT3 | rs297808 | 5 | 168483143 | A | G |
| 3487 | SLIT3 | rs17070834 | 5 | 168486834 | T | C |
| 3488 | SLIT3 | rs188431 | S | 168488444 | A | C |
| 3489 | SLIT3 | rs2668046 | 5 | 168491381 | G | A |
| 3490 | SLIT3 | rs888688 | 5 | 168492757 | T | C |
| 3491 | SLIT3 | rs1421765 | 5 | 168493973 | G | A |
| 3492 | SLIT3 | rs1421766 | 5 | 168494089 | G | A |
| 3493 | SLIT3 | rs10071654 | 5 | 168495596 | C | A |
| 3494 | SLIT3 | rs2338297 | 5 | 168497136 | T | C |
| 3495 | SLIT3 | rs12654605 | 5 | 168497258 | A | C |
| 3496 | SLIT3 | rs12522564 | 5 | 168503024 | G | A |
| 3497 | SLIT3 | rs297836 | 5 | 168503883 | G | A |
| 3498 | SLIT3 | rs297832 | 5 | 168508529 | C | T |
| 3499 | SLIT3 | rs6884996 | 5 | 168509507 | C | T |
| 3500 | SLIT3 | rs297827 | 5 | 168512280 | G | A |
| 3501 | SLIT3 | rs10475901 | 5 | 168512421 | G | A |
| 3502 | SLIT3 | rs2668062 | 5 | 168514917 | C | T |
| 3503 | SLIT3 | rs297812 | 5 | 168516544 | A | G |
| 3504 | SLIT3 | rs190935 | 5 | 168517041 | C | T |
| 3505 | SLIT3 | rs4868327 | 5 | 168519906 | C | — |
| 3506 | SLIT3 | rs7722860 | 5 | 168521504 | G | A |
| 3507 | SLIT3 | rs297820 | 5 | 168525875 | G | T |
| 3508 | SLIT3 | rs297823 | 5 | 168527581 | C | T |
| 3509 | SLIT3 | rs7700961 | 5 | 168528015 | T | C |
| 3510 | SLIT3 | rs10462980 | 5 | 168529461 | T | G |
| 3511 | SLIT3 | rs9313446 | 5 | 168530765 | G | A |
| 3512 | SLIT3 | rs10516061 | 5 | 168532207 | G | A |
| 3513 | SLIT3 | rs1006329 | 5 | 168532429 | A | G |
| 3514 | SLIT3 | rs11745045 | 5 | 168533349 | C | T |
| 3515 | SLIT3 | rs11950412 | 5 | 168535682 | A | G |
| 3516 | SLIT3 | rs12163927 | 5 | 168540878 | G | A |
| 3517 | SLIT3 | rs12653232 | 5 | 168542268 | C | — |
| 3518 | SLIT3 | rs297879 | 5 | 168543366 | C | A |
| 3519 | SLIT3 | rs9313447 | 5 | 168544310 | G | A |
| 3520 | SLIT3 | rs297883 | 5 | 168545254 | A | G |
| 3521 | SLIT3 | rs297884 | 5 | 168545413 | A | C |
| 3522 | SLIT3 | rs12513707 | 5 | 168545860 | C | T |
| 3523 | SLIT3 | rs12515660 | 5 | 168553502 | G | A |
| 3524 | SLIT3 | rs297838 | 5 | 168554351 | C | T |
| 3525 | SLIT3 | rs744579 | 5 | 168558819 | T | C |
| 3526 | SLIT3 | rs297853 | 5 | 168561256 | G | A |
| 3527 | SLIT3 | rs12656908 | 5 | 168564595 | G | A |
| 3528 | SLIT3 | rs17643060 | 5 | 168565607 | T | C |
| 3529 | SLIT3 | rs11738097 | 5 | 168566025 | T | G |
| 3530 | SLIT3 | rs10059292 | 5 | 168570125 | T | C |
| 3531 | SLIT3 | rs1345592 | 5 | 168573568 | A | G |
| 3532 | SLIT3 | rs4868388 | 5 | 168575142 | A | C |
| 3533 | SLIT3 | rs6867453 | 5 | 168576941 | G | T |
| 3534 | SLIT3 | rs716866 | 5 | 168578301 | T | C |
| 3535 | SLIT3 | rs17643126 | 5 | 168579082 | C | T |
| 3536 | SLIT3 | rs10866632 | 5 | 168582199 | T | G |
| 3537 | SLIT3 | rs978902 | 5 | 168582774 | C | T |
| 3538 | SLIT3 | rs12653501 | 5 | 168583615 | T | C |
| 3539 | SLIT3 | rs6868507 | 5 | 168585455 | T | C |
| 3540 | SLIT3 | rs6893511 | 5 | 168586691 | C | A |
| 3541 | SLIT3 | rs12515501 | 5 | 168590447 | A | G |
| 3542 | SLIT3 | rs10079601 | 5 | 168590744 | T | C |
| 3543 | SLIT3 | rs10043015 | 5 | 168590949 | T | C |
| 3544 | SLIT3 | rs10056028 | 5 | 168595873 | G | A |
| 3545 | SLIT3 | rs7719642 | 5 | 168596521 | T | C |
| 3546 | SLIT3 | rs10038332 | 5 | 168605984 | T | C |
| 3547 | SLIT3 | rs972031 | 5 | 168610371 | C | T |
| 3548 | SLIT3 | rs1421764 | 5 | 168610826 | C | T |
| 3549 | SLIT3 | rs11954621 | 5 | 168612481 | G | A |
| 3550 | SLIT3 | rs7716873 | 5 | 168613887 | G | A |
| 3551 | SLIT3 | rs11134564 | 5 | 168621228 | A | G |
| 3552 | SLIT3 | rs6555852 | 5 | 168630385 | T | G |
| 3553 | SLIT3 | rs12521978 | 5 | 168632530 | G | A |
| 3554 | SLIT3 | rs7712111 | 5 | 168635069 | A | G |
| 3555 | SLIT3 | rs754431 | 5 | 168636529 | G | A |
| 3556 | SLIT3 | rs11134568 | 5 | 168640140 | C | T |
| 3557 | SLIT3 | rs888781 | 5 | 168641645 | C | T |
| 3558 | SLIT3 | rs751347 | 5 | 168643048 | A | G |
| 3559 | SLIT3 | rs751348 | 5 | 168643201 | C | T |
| 3560 | SLIT3 | rs7380303 | 5 | 168655515 | T | C |
| 3561 | UNC5A | rs551657 | 5 | 176166398 | G | A |
| 3562 | UNC5A | rs692403 | 5 | 176167681 | A | G |
| 3563 | UNC5A | rs692653 | 5 | 176172372 | C | T |
| 3564 | UNC5A | rs2940520 | 5 | 176174931 | A | G |
| 3565 | UNC5A | rs692825 | 5 | 176188263 | A | G |
| 3566 | UNC5A | rs12521024 | 5 | 176195033 | G | A |
| 3567 | UNC5A | rs480782 | 5 | 176205976 | T | C |
| 3568 | UNC5A | rs7380999 | 5 | 176226309 | T | C |
| 3569 | UNC5A | rs11954962 | 5 | 176227501 | G | T |
| 3570 | UNC5A | rs478253 | 5 | 176238284 | G | A |
| 3571 | UNC5A | rs10475632 | 5 | 176238580 | G | A |
| 3572 | UNC5A | rs3736432 | 5 | 176240110 | G | A |
| 3573 | UNC5A | rs539822 | 5 | 176242770 | C | T |
| 3574 | UNC5A | rs4976661 | 5 | 176242804 | T | C |
| 3575 | EPHA7 | rs9354002 | 6 | 94004028 | T | C |
| 3576 | EPHA7 | rs3823231 | 6 | 94016082 | A | G |
| 3577 | EPHA7 | rs1386274 | 6 | 94018292 | T | C |
| 3578 | EPHA7 | rs404201 | 6 | 94018613 | C | T |
| 3579 | EPHA7 | rs17447037 | 6 | 94019483 | C | T |
| 3580 | EPHA7 | rs345732 | 6 | 94023205 | A | — |
| 3581 | EPHA7 | rs10806474 | 6 | 94034629 | T | C |
| 3582 | EPHA7 | rs345713 | 6 | 94038845 | C | T |
| 3583 | EPHA7 | rs345718 | 6 | 94043821 | T | C |
| 3584 | EPHA7 | rs17447791 | 6 | 94052175 | A | G |
| 3585 | EPHA7 | rs967520 | 6 | 94057234 | A | C |
| 3586 | EPHA7 | rs346462 | 6 | 94061206 | A | G |
| 3587 | EPHA7 | rs10944662 | 6 | 94061983 | C | T |
| 3588 | EPHA7 | rs9363058 | 6 | 94065256 | C | T |
| 3589 | EPHA7 | rs345716 | 6 | 94068962 | A | C |
| 3590 | EPHA7 | rs351321 | 6 | 94076284 | C | T |
| 3591 | EPHA7 | rs164294 | 6 | 94082305 | G | A |
| 3592 | EPHA7 | rs164298 | 6 | 94085686 | G | A |
| 3593 | EPHA7 | rs164301 | 6 | 94086536 | G | T |
| 3594 | EPHA7 | rs164302 | 6 | 94088372 | G | A |
| 3595 | EPHA7 | rs9294570 | 6 | 94093451 | G | A |
| 3596 | EPHA7 | rs16871170 | 6 | 94097830 | C | T |
| 3597 | EPHA7 | rs17687366 | 6 | 94101390 | C | T |
| 3598 | EPHA7 | rs13200186 | 6 | 94106055 | C | T |
| 3599 | EPHA7 | rs4706437 | 6 | 94107893 | G | A |
| 3600 | EPHA7 | rs13200697 | 6 | 94108440 | C | T |
| 3601 | EPHA7 | rs13203837 | 6 | 94108633 | A | C |
| 3602 | EPHA7 | rs168435 | 6 | 94110330 | G | A |
| 3603 | EPHA7 | rs4707795 | 6 | 94111589 | G | A |
| 3604 | EPHA7 | rs361505 | 6 | 94113923 | T | C |
| 3605 | EPHA7 | rs168436 | 6 | 94116271 | C | T |
| 3606 | EPHA7 | rs164538 | 6 | 94117090 | T | C |
| 3607 | EPHA7 | rs164539 | 6 | 94118126 | G | T |
| 3608 | EPHA7 | rs17688085 | 6 | 94118775 | G | A |
| 3609 | EPHA7 | rs164540 | 6 | 94121127 | A | G |
| 3610 | EPHA7 | rs164544 | 6 | 94124702 | A | G |
| 3611 | EPHA7 | rs164548 | 6 | 94128329 | G | A |
| 3612 | EPHA7 | rs9354011 | 6 | 94132168 | G | A |
| 3613 | EPHA7 | rs3778470 | 6 | 94132405 | C | T |
| 3614 | EPHA7 | rs351328 | 6 | 94139844 | C | A |
| 3615 | EPHA7 | rs3823229 | 6 | 94140513 | C | T |
| 3616 | EPHA7 | rs3799807 | 6 | 94146797 | G | A |
| 3617 | EPHA7 | rs12661215 | 6 | 94155840 | C | T |
| 3618 | EPHA7 | rs2052636 | 6 | 94160347 | G | A |
| 3619 | EPHA7 | rs549965 | 6 | 94161248 | C | T |
| 3620 | EPHA7 | rs564158 | 6 | 94165594 | A | G |
| 3621 | EPHA7 | rs3799792 | 6 | 94174267 | A | G |
| 3622 | EPHA7 | rs2278107 | 6 | 94177360 | T | C |
| 3623 | EPHA7 | rs575554 | 6 | 94182713 | G | A |
| 3624 | EPHA7 | rs9452315 | 6 | 94189958 | G | — |
| 3625 | EPHA7 | rs519420 | 6 | 94190648 | A | G |
| 3626 | FYN | rs1726131 | 6 | 112084553 | G | A |
| 3627 | FYN | rs6916861 | 6 | 112088950 | A | C |
| 3628 | FYN | rs1794841 | 6 | 112090115 | G | A |
| 3629 | FYN | rs6919400 | 6 | 112098014 | C | T |
| 3630 | FYN | rs2282855 | 6 | 112098504 | A | G |
| 3631 | FYN | rs13203885 | 6 | 112101820 | T | C |
| 3632 | FYN | rs9374274 | 6 | 112103112 | G | A |
| 3633 | FYN | rs1358750 | 6 | 112106275 | G | A |
| 3634 | FYN | rs7746279 | 6 | 112106855 | G | A |
| 3635 | FYN | rs4947143 | 6 | 112108819 | T | C |
| 3636 | FYN | rs706862 | 6 | 112110441 | G | A |
| 3637 | FYN | rs2237259 | 6 | 112113242 | C | T |
| 3638 | FYN | rs1998038 | 6 | 112113688 | C | T |
| 3639 | FYN | rs11153311 | 6 | 112116018 | A | G |
| 3640 | FYN | rs809193 | 6 | 112127107 | A | G |
| 3641 | FYN | rs3752545 | 6 | 112130955 | A | C |
| 3642 | FYN | rs1022648 | 6 | 112136297 | T | C |
| 3643 | FYN | rs809192 | 6 | 112141695 | G | A |
| 3644 | FYN | rs706885 | 6 | 112142550 | A | C |
| 3645 | FYN | rs17704348 | 6 | 112144178 | T | C |
| 3646 | FYN | rs706895 | 6 | 112148052 | T | C |
| 3647 | FYN | rs697642 | 6 | 112159798 | G | A |
| 3648 | FYN | rs6930230 | 6 | 112170175 | T | C |
| 3649 | FYN | rs6925303 | 6 | 112173125 | A | G |
| 3650 | FYN | rs6938050 | 6 | 112175075 | G | A |
| 3651 | FYN | rs6910116 | 6 | 112182621 | A | G |
| 3652 | FYN | rs9387033 | 6 | 112184100 | T | C |
| 3653 | FYN | rs1465061 | 6 | 112188309 | G | A |
| 3654 | FYN | rs6914091 | 6 | 112188428 | C | T |
| 3655 | FYN | rs11964650 | 6 | 112188631 | C | T |
| 3656 | FYN | rs13218316 | 6 | 112189727 | C | T |
| 3657 | FYN | rs11963612 | 6 | 112203038 | T | C |
| 3658 | FYN | rs11153318 | 6 | 112204293 | G | A |
| 3659 | FYN | rs9384805 | 6 | 112208418 | A | C |
| 3660 | FYN | rs2148710 | 6 | 112228919 | C | T |
| 3661 | FYN | rs1327200 | 6 | 112229066 | C | T |
| 3662 | FYN | rs7761230 | 6 | 112232996 | G | A |
| 3663 | FYN | rs17072912 | 6 | 112233032 | G | A |
| 3664 | FYN | rs12526016 | 6 | 112234032 | T | C |
| 3665 | FYN | rs7757969 | 6 | 112238725 | T | C |
| 3666 | FYN | rs2182644 | 6 | 112245575 | G | A |
| 3667 | FYN | rs9372313 | 6 | 112248783 | C | T |
| 3668 | FYN | rs1327202 | 6 | 112259108 | G | A |
| 3669 | FYN | rs11961774 | 6 | 112262166 | A | G |
| 3670 | FYN | rs6923217 | 6 | 112263790 | T | C |
| 3671 | FYN | rs7749147 | 6 | 112271006 | G | A |
| 3672 | FYN | rs12661059 | 6 | 112271895 | T | C |
| 3673 | FYN | rs9487737 | 6 | 112282210 | C | T |
| 3674 | FYN | rs10499061 | 6 | 112288723 | C | T |
| 3675 | FYN | rs6924754 | 6 | 112290506 | G | A |
| 3676 | FYN | rs9320379 | 6 | 112291314 | T | C |
| 3677 | FYN | rs2024832 | 6 | 112296405 | T | G |
| 3678 | FYN | rs1409837 | 6 | 112302304 | T | C |
| 3679 | RAC1 | rs2689420 | 7 | 6376846 | A | G |
| 3680 | RAC1 | rs4724800 | 7 | 6377053 | A | G |
| 3681 | RAC1 | rs7784465 | 7 | 6384800 | T | C |
| 3682 | RAC1 | rs2347338 | 7 | 6384803 | C | T |
| 3683 | RAC1 | rs836475 | 7 | 6400457 | G | A |
| 3684 | RAC1 | rs836474 | 7 | 6400612 | G | T |
| 3685 | RAC1 | rs836472 | 7 | 6401429 | C | T |
| 3686 | RAC1 | rs10951983 | 7 | 6412552 | A | G |
| 3687 | RAC1 | rs836556 | 7 | 6413566 | T | C |
| 3688 | LIMK1 | rs2855726 | 7 | 73155050 | G | A |
| 3689 | LIMK1 | rs2269082 | 7 | 73157811 | T | C |
| 3690 | LIMK1 | rs2269081 | 7 | 73157823 | A | C |
| 3691 | LIMK1 | rs150862 | 7 | 73162143 | A | G |
| 3692 | LIMK1 | rs2285026 | 7 | 73168340 | C | T |
| 3693 | LIMK1 | rs178413 | 7 | 73176262 | C | T |
| 3694 | GNAI1 | rs2428469 | 7 | 79603558 | A | C |
| 3695 | GNAI1 | rs12706644 | 7 | 79609963 | T | C |
| 3696 | GNAI1 | rs2523189 | 7 | 79614364 | C | T |
| 3697 | GNAI1 | rs10274707 | 7 | 79618228 | C | T |
| 3698 | GNAI1 | rs2714449 | 7 | 79629883 | C | T |
| 3699 | GNAI1 | rs17806203 | 7 | 79630542 | G | A |
| 3700 | GNAI1 | rs2523194 | 7 | 79634751 | A | G |
| 3701 | GNAI1 | rs1468242 | 7 | 79636560 | T | C |
| 3702 | GNAI1 | rs11980616 | 7 | 79637549 | A | G |
| 3703 | GNAI1 | rs916905 | 7 | 79638283 | T | C |
| 3704 | GNAI1 | rs10230368 | 7 | 79647920 | A | G |
| 3705 | GNAI1 | rs6956455 | 7 | 79652863 | G | A |
| 3706 | GNAI1 | rs4512317 | 7 | 79655885 | A | C |
| 3707 | GNAI1 | rs6467043 | 7 | 79660339 | T | C |
| 3708 | GNAI1 | rs17153557 | 7 | 79663356 | G | — |
| 3709 | GNAI1 | rs10272499 | 7 | 79667780 | C | T |
| 3710 | GNAI1 | rs17245731 | 7 | 79670569 | A | G |
| 3711 | GNAI1 | rs10238438 | 7 | 79679890 | A | G |
| 3712 | GNAI1 | rs2886611 | 7 | 79681241 | C | T |
| 3713 | GNAI1 | rs9244 | 7 | 79685251 | T | C |
| 3714 | GNAI1 | rs6973616 | 7 | 79688271 | C | T |
| 3715 | GNAI1 | rs10247757 | 7 | 79691383 | C | A |
| 3716 | SEMA3C | rs1527470 | 7 | 80208765 | A | G |
| 3717 | SEMA3C | rs10499863 | 7 | 80209630 | T | — |
| 3718 | SEMA3C | rs1358340 | 7 | 80213122 | C | A |
| 3719 | SEMA3C | rs2886792 | 7 | 80214197 | C | T |
| 3720 | SEMA3C | rs3778674 | 7 | 80220528 | C | A |
| 3721 | SEMA3C | rs6978637 | 7 | 80223923 | G | A |
| 3722 | SEMA3C | rs17147989 | 7 | 80224536 | T | C |
| 3723 | SEMA3C | rs10233114 | 7 | 80225296 | T | G |
| 3724 | SEMA3C | rs1058425 | 7 | 80225702 | G | A |
| 3725 | SEMA3C | rs3807094 | 7 | 80226072 | G | A |
| 3726 | SEMA3C | rs3807101 | 7 | 80231354 | C | T |
| 3727 | SEMA3C | rs10266458 | 7 | 80239406 | A | G |
| 3728 | SEMA3C | rs13222151 | 7 | 80240490 | G | A |
| 3729 | SEMA3C | rs12671126 | 7 | 80241121 | C | T |
| 3730 | SEMA3C | rs13224811 | 7 | 80245737 | C | T |
| 3731 | SEMA3C | rs12673722 | 7 | 80247480 | A | C |
| 3732 | SEMA3C | rs3778677 | 7 | 80249040 | C | T |
| 3733 | SEMA3C | rs7803929 | 7 | 80255746 | A | G |
| 3734 | SEMA3C | rs17275986 | 7 | 80256625 | A | G |
| 3735 | SEMA3C | rs13242982 | 7 | 80263794 | T | C |
| 3736 | SEMA3C | rs3807109 | 7 | 80266361 | G | T |
| 3737 | SEMA3C | rs3807110 | 7 | 80266426 | T | C |
| 3738 | SEMA3C | rs17154508 | 7 | 80289376 | C | T |
| 3739 | SEMA3C | rs12535655 | 7 | 80289751 | T | C |
| 3740 | SEMA3C | rs12670551 | 7 | 80291680 | A | G |
| 3741 | SEMA3C | rs12155128 | 7 | 80292871 | T | C |
| 3742 | SEMA3C | rs2040908 | 7 | 80297952 | A | C |
| 3743 | SEMA3C | rs886660 | 7 | 80300775 | G | A |
| 3744 | SEMA3C | rs929346 | 7 | 80300840 | C | T |
| 3745 | SEMA3C | rs10215990 | 7 | 80303494 | A | C |
| 3746 | SEMA3C | rs11767508 | 7 | 80306063 | T | C |
| 3747 | SEMA3C | rs17228184 | 7 | 80306737 | A | G |
| 3748 | SEMA3C | rs4731861 | 7 | 80309471 | C | T |
| 3749 | SEMA3C | rs1016840 | 7 | 80311982 | T | C |
| 3750 | SEMA3C | rs12707038 | 7 | 80316928 | T | C |
| 3751 | SEMA3C | rs10954378 | 7 | 80326004 | G | A |
| 3752 | SEMA3C | rs765764 | 7 | 80346358 | T | C |
| 3753 | SEMA3C | rs6956461 | 7 | 80357190 | T | C |
| 3754 | SEMA3C | rs17154583 | 7 | 80360842 | G | A |
| 3755 | SEMA3C | rs7779029 | 7 | 80370048 | T | C |
| 3756 | SEMA3C | rs7793555 | 7 | 80384681 | G | A |
| 3757 | SEMA3C | rs2057880 | 7 | 80387869 | C | A |
| 3758 | SEMA3E | rs17157534 | 7 | 82827664 | T | — |
| 3759 | SEMA3E | rs2371656 | 7 | 82828913 | A | G |
| 3760 | SEMA3E | rs10273103 | 7 | 82847878 | A | G |
| 3761 | SEMA3E | rs3801479 | 7 | 82852932 | A | G |
| 3762 | SEMA3E | rs2709922 | 7 | 82863254 | G | A |
| 3763 | SEMA3E | rs7777731 | 7 | 82865281 | A | G |
| 3764 | SEMA3E | rs17285647 | 7 | 82871577 | T | C |
| 3765 | SEMA3E | rs2722983 | 7 | 82874830 | T | C |
| 3766 | SEMA3E | rs2709963 | 7 | 82879280 | T | — |
| 3767 | SEMA3E | rs2709894 | 7 | 82885132 | G | A |
| 3768 | SEMA3E | rs2722964 | 7 | 82896763 | C | T |
| 3769 | SEMA3E | rs12536989 | 7 | 82909807 | A | C |
| 3770 | SEMA3E | rs2713174 | 7 | 82913893 | T | C |
| 3771 | SEMA3E | rs2723022 | 7 | 82917345 | A | G |
| 3772 | SEMA3E | rs2713178 | 7 | 82925129 | C | A |
| 3773 | SEMA3E | rs10261533 | 7 | 82937000 | A | G |
| 3774 | SEMA3E | rs2722990 | 7 | 82939420 | T | G |
| 3775 | SEMA3E | rs4732512 | 7 | 82941991 | G | A |
| 3776 | SEMA3E | rs10270844 | 7 | 82946973 | C | T |
| 3777 | SEMA3E | rs2255872 | 7 | 82951619 | A | G |
| 3778 | SEMA3E | rs9640942 | 7 | 82956811 | G | T |
| 3779 | SEMA3E | rs2713192 | 7 | 82962946 | C | T |
| 3780 | SEMA3E | rs3801516 | 7 | 82964795 | T | C |
| 3781 | SEMA3E | rs11982066 | 7 | 82972395 | T | C |
| 3782 | SEMA3E | rs10485857 | 7 | 82978148 | A | C |
| 3783 | SEMA3E | rs2535372 | 7 | 82982048 | T | C |
| 3784 | SEMA3E | rs12530588 | 7 | 82986148 | G | A |
| 3785 | SEMA3E | rs1860610 | 7 | 82987500 | A | C |
| 3786 | SEMA3E | rs7779140 | 7 | 82987671 | G | A |
| 3787 | SEMA3E | rs2535368 | 7 | 82995143 | G | A |
| 3788 | SEMA3E | rs17505926 | 7 | 82996280 | T | C |
| 3789 | SEMA3E | rs988578 | 7 | 82999150 | C | T |
| 3790 | SEMA3E | rs7810691 | 7 | 83005264 | A | G |
| 3791 | SEMA3E | rs10255137 | 7 | 83008913 | A | G |
| 3792 | SEMA3E | rs17447180 | 7 | 83009617 | C | T |
| 3793 | SEMA3E | rs1693381 | 7 | 83011868 | T | C |
| 3794 | SEMA3E | rs1639309 | 7 | 83012299 | C | T |
| 3795 | SEMA3E | rs2058346 | 7 | 83012364 | G | A |
| 3796 | SEMA3E | rs1353615 | 7 | 83013723 | C | A |
| 3797 | SEMA3E | rs1024701 | 7 | 83014058 | T | C |
| 3798 | SEMA3E | rs1693389 | 7 | 83014198 | T | C |
| 3799 | SEMA3E | rs1639304 | 7 | 83017558 | A | G |
| 3800 | SEMA3E | rs2371784 | 7 | 83021659 | G | A |
| 3801 | SEMA3E | rs215276 | 7 | 83031305 | T | C |
| 3802 | SEMA3E | rs215288 | 7 | 83042719 | T | C |
| 3803 | SEMA3E | rs1979768 | 7 | 83044509 | G | T |
| 3804 | SEMA3E | rs215294 | 7 | 83046638 | C | T |
| 3805 | SEMA3E | rs215300 | 7 | 83050721 | C | T |
| 3806 | SEMA3E | rs215311 | 7 | 83057203 | A | G |
| 3807 | SEMA3E | rs215314 | 7 | 83059944 | C | T |
| 3808 | SEMA3E | rs727549 | 7 | 83065910 | A | G |
| 3809 | SEMA3E | rs3823906 | 7 | 83067501 | A | C |
| 3810 | SEMA3E | rs10246477 | 7 | 83103936 | G | A |
| 3811 | SEMA3E | rs10246487 | 7 | 83103951 | G | — |
| 3812 | SEMA3E | rs536299 | 7 | 83105919 | C | A |
| 3813 | SEMA3E | rs538007 | 7 | 83106050 | C | T |
| 3814 | SEMA3E | rs17157933 | 7 | 83106939 | G | A |
| 3815 | SEMA3E | rs1391064 | 7 | 83107187 | C | A |
| 3816 | SEMA3E | rs3757621 | 7 | 83107429 | G | A |
| 3817 | SEMA3E | rs302119 | 7 | 83113319 | G | A |
| 3818 | SEMA3E | rs302113 | 7 | 83118134 | G | T |
| 3819 | SEMA3A | rs797823 | 7 | 83424650 | T | C |
| 3820 | SEMA3A | rs797821 | 7 | 83428788 | G | A |
| 3821 | SEMA3A | rs797810 | 7 | 83434619 | G | A |
| 3822 | SEMA3A | rs740948 | 7 | 83437271 | C | T |
| 3823 | SEMA3A | rs701320 | 7 | 83444454 | T | C |
| 3824 | SEMA3A | rs740947 | 7 | 83454464 | A | G |
| 3825 | SEMA3A | rs3735513 | 7 | 83469312 | T | C |
| 3826 | SEMA3A | rs7804122 | 7 | 83472649 | A | G |
| 3827 | SEMA3A | rs4732529 | 7 | 83473522 | C | T |
| 3828 | SEMA3A | rs10499878 | 7 | 83477623 | A | G |
| 3829 | SEMA3A | rs17158505 | 7 | 83480491 | G | T |
| 3830 | SEMA3A | rs4732531 | 7 | 83482890 | A | G |
| 3831 | SEMA3A | rs1534075 | 7 | 83482921 | T | G |
| 3832 | SEMA3A | rs6949238 | 7 | 83484752 | T | C |
| 3833 | SEMA3A | rs6976211 | 7 | 83489790 | C | T |
| 3834 | SEMA3A | rs17158529 | 7 | 83497513 | C | T |
| 3835 | SEMA3A | rs1533995 | 7 | 83502084 | G | A |
| 3836 | SEMA3A | rs1404473 | 7 | 83505109 | T | C |
| 3837 | SEMA3A | rs4355720 | 7 | 83516888 | A | G |
| 3838 | SEMA3A | rs2527032 | 7 | 83525558 | C | T |
| 3839 | SEMA3A | rs2527039 | 7 | 83527550 | C | T |
| 3840 | SEMA3A | rs2189849 | 7 | 83529507 | C | A |
| 3841 | SEMA3A | rs2527031 | 7 | 83536113 | T | C |
| 3842 | SEMA3A | rs10243933 | 7 | 83537423 | T | G |
| 3843 | SEMA3A | rs17296151 | 7 | 83537669 | A | G |
| 3844 | SEMA3A | rs10488266 | 7 | 83539613 | T | C |
| 3845 | SEMA3A | rs2691694 | 7 | 83540412 | G | A |
| 3846 | SEMA3A | rs17158583 | 7 | 83540955 | G | A |
| 3847 | SEMA3A | rs1012974 | 7 | 83541237 | A | G |
| 3848 | SEMA3A | rs2691699 | 7 | 83554956 | A | G |
| 3849 | SEMA3A | rs3801615 | 7 | 83558710 | C | T |
| 3850 | SEMA3A | rs2527038 | 7 | 83559475 | A | C |
| 3851 | SEMA3A | rs3823911 | 7 | 83570210 | T | G |
| 3852 | SEMA3A | rs7801391 | 7 | 83571857 | A | C |
| 3853 | SEMA3A | rs917090 | 7 | 83571953 | T | C |
| 3854 | SEMA3A | rs764116 | 7 | 83576417 | T | C |
| 3855 | SEMA3A | rs2372032 | 7 | 83580426 | G | T |
| 3856 | SEMA3A | rs929334 | 7 | 83582309 | C | T |
| 3857 | SEMA3A | rs13222995 | 7 | 83583311 | A | G |
| 3858 | SEMA3A | rs2372033 | 7 | 83584249 | T | C |
| 3859 | SEMA3A | rs2067693 | 7 | 83597506 | A | G |
| 3860 | SEMA3A | rs10488270 | 7 | 83608284 | C | A |
| 3861 | SEMA3A | rs7781126 | 7 | 83612435 | C | T |
| 3862 | SEMA3A | rs6978184 | 7 | 83616296 | T | C |
| 3863 | SEMA3A | rs7781152 | 7 | 83620371 | C | T |
| 3864 | SEMA3A | rs464 | 7 | 83624396 | C | T |
| 3865 | SEMA3A | rs17158686 | 7 | 83632692 | T | G |
| 3866 | SEMA3A | rs11974667 | 7 | 83634556 | C | A |
| 3867 | SEMA3A | rs12666837 | 7 | 83639293 | G | A |
| 3868 | SEMA3A | rs10261069 | 7 | 83639588 | T | C |
| 3869 | SEMA3A | rs7811968 | 7 | 83641563 | C | T |
| 3870 | SEMA3A | rs12707622 | 7 | 83644670 | C | A |
| 3871 | SEMA3A | rs13224698 | 7 | 83647047 | A | C |
| 3872 | SEMA3A | rs12707628 | 7 | 83655831 | C | T |
| 3873 | SEMA3A | rs11976488 | 7 | 83657451 | A | G |
| 3874 | SEMA3A | rs996033 | 7 | 83664731 | A | G |
| 3875 | SEMA3A | rs12673519 | 7 | 83665936 | G | A |
| 3876 | SEMA3D | rs2286184 | 7 | 84468452 | C | T |
| 3877 | SEMA3D | rs2286185 | 7 | 84469322 | C | T |
| 3878 | SEMA3D | rs17558985 | 7 | 84481878 | G | T |
| 3879 | SEMA3D | rs17559084 | 7 | 84482436 | C | T |
| 3880 | SEMA3D | rs2158727 | 7 | 84506381 | A | C |
| 3881 | SEMA3D | rs10486847 | 7 | 84512933 | G | A |
| 3882 | SEMA3D | rs12531511 | 7 | 84529293 | T | C |
| 3883 | SEMA3D | rs2240394 | 7 | 84530877 | T | C |
| 3884 | SEMA3D | rs6966472 | 7 | 84537323 | T | C |
| 3885 | SEMA3D | rs12673176 | 7 | 84538569 | G | T |
| 3886 | SEMA3D | rs17560655 | 7 | 84542383 | T | C |
| 3887 | SEMA3D | rs6962019 | 7 | 84545861 | C | T |
| 3888 | SEMA3D | rs10260011 | 7 | 84547292 | C | T |
| 3889 | SEMA3D | rs12536409 | 7 | 84559063 | G | T |
| 3890 | SEMA3D | rs17641482 | 7 | 84571074 | T | C |
| 3891 | SEMA3D | rs10486848 | 7 | 84575619 | G | A |
| 3892 | SEMA3D | rs1029563 | 7 | 84576431 | T | G |
| 3893 | SEMA3D | rs993312 | 7 | 84585780 | C | A |
| 3894 | SEMA3D | rs17647278 | 7 | 84587114 | G | T |
| 3895 | SEMA3D | rs2190208 | 7 | 84589887 | T | C |
| 3896 | SEMA3D | rs6970924 | 7 | 84590157 | A | C |
| 3897 | SEMA3D | rs11762367 | 7 | 84590361 | C | T |
| 3898 | SEMA3D | rs17647464 | 7 | 84592087 | G | A |
| 3899 | EPHB4 | rs144173 | 7 | 100254186 | C | T |
| 3900 | EPHB4 | rs2250818 | 7 | 100260092 | G | A |
| 3901 | EPHB4 | rs314313 | 7 | 100261301 | A | G |
| 3902 | EPHB4 | rs2247445 | 7 | 100265199 | C | T |
| 3903 | MET | rs38840 | 7 | 116100222 | C | T |
| 3904 | MET | rs714180 | 7 | 116106238 | G | A |
| 3905 | MET | rs38841 | 7 | 116107162 | A | G |
| 3906 | MET | rs38845 | 7 | 116109038 | G | A |
| 3907 | MET | rs10487353 | 7 | 116111172 | G | T |
| 3908 | MET | rs722134 | 7 | 116120945 | T | C |
| 3909 | MET | rs2237712 | 7 | 116125894 | A | G |
| 3910 | MET | rs2299436 | 7 | 116128619 | A | G |
| 3911 | MET | rs2299437 | 7 | 116128724 | G | A |
| 3912 | MET | rs17138948 | 7 | 116131546 | C | T |
| 3913 | MET | rs10243024 | 7 | 116133839 | G | A |
| 3914 | MET | rs38855 | 7 | 116145280 | A | G |
| 3915 | MET | rs38857 | 7 | 116152649 | C | T |
| 3916 | MET | rs10232307 | 7 | 116156423 | G | A |
| 3917 | MET | rs13223756 | 7 | 116184808 | A | G |
| 3918 | MET | rs10215153 | 7 | 116186367 | G | A |
| 3919 | MET | rs17138983 | 7 | 116189969 | A | G |
| 3920 | MET | rs2237717 | 7 | 116192623 | C | T |
| 3921 | MET | rs2299440 | 7 | 116203563 | C | T |
| 3922 | MET | rs17526983 | 7 | 116203659 | G | A |
| 3923 | MET | rs2402118 | 7 | 116215809 | C | A |
| 3924 | MET | rs193686 | 7 | 116218663 | T | C |
| 3925 | MET | rs193688 | 7 | 116220763 | T | C |
| 3926 | MET | rs183642 | 7 | 116220866 | G | A |
| 3927 | MET | rs41741 | 7 | 116225747 | T | G |
| 3928 | EPHB6 | rs8177130 | 7 | 142269523 | C | A |
| 3929 | EPHB6 | rs6947538 | 7 | 142272858 | T | C |
| 3930 | EPHB6 | rs8177146 | 7 | 142273375 | T | G |
| 3931 | EPHB6 | rs4987667 | 7 | 142283030 | A | G |
| 3932 | CDK5 | rs2288645 | 7 | 150378679 | G | A |
| 3933 | CDKS | rs2069459 | 7 | 150382523 | G | T |
| 3934 | PPP3CC | rs12546391 | 8 | 22351173 | T | C |
| 3935 | PPP3CC | rs7837713 | 8 | 22375143 | T | C |
| 3936 | PPP3CC | rs2443502 | 8 | 22396444 | G | A |
| 3937 | PPP3CC | rs2469749 | 8 | 22413543 | C | T |
| 3938 | PPP3CC | rs2461491 | 8 | 22417197 | T | C |
| 3939 | PPP3CC | rs2461489 | 8 | 22426198 | C | T |
| 3940 | PPP3CC | rs17733133 | 8 | 22441006 | T | C |
| 3941 | PPP3CC | rs2461483 | 8 | 22442424 | C | T |
| 3942 | PPP3CC | rs17733355 | 8 | 22444802 | G | A |
| 3943 | DPYSL2 | rs401004 | 8 | 26487383 | G | A |
| 3944 | DPYSL2 | rs6998628 | 8 | 26489480 | G | A |
| 3945 | DPYSL2 | rs6983262 | 8 | 26489803 | C | T |
| 3946 | DPYSL2 | rs920697 | 8 | 26492993 | A | G |
| 3947 | DPYSL2 | rs1662248 | 8 | 26499553 | T | G |
| 3948 | DPYSL2 | rs327235 | 8 | 26500471 | G | A |
| 3949 | DPYSL2 | rs327234 | 8 | 26500586 | A | G |
| 3950 | DPYSL2 | rs7825468 | 8 | 26506390 | A | G |
| 3951 | DPYSL2 | rs5029306 | 8 | 26509022 | C | T |
| 3952 | DPYSL2 | rs327232 | 8 | 26511632 | C | T |
| 3953 | DPYSL2 | rs7834325 | 8 | 26517277 | G | T |
| 3954 | DPYSL2 | rs327229 | 8 | 26518078 | G | A |
| 3955 | DPYSL2 | rs11780026 | 8 | 26518541 | A | G |
| 3956 | DPYSL2 | rs13249543 | 8 | 26519261 | A | G |
| 3957 | DPYSL2 | rs11775967 | 8 | 26522777 | C | A |
| 3958 | DPYSL2 | rs7011131 | 8 | 26526139 | T | G |
| 3959 | DPYSL2 | rs17321828 | 8 | 26527954 | G | A |
| 3960 | DPYSL2 | rs4733013 | 8 | 26528572 | G | A |
| 3961 | DPYSL2 | rs2585459 | 8 | 26529541 | A | G |
| 3962 | DPYSL2 | rs403185 | 8 | 26532638 | G | A |
| 3963 | DPYSL2 | rs1774146 | 8 | 26536278 | T | C |
| 3964 | DPYSL2 | rs7829347 | 8 | 26536534 | T | C |
| 3965 | DPYSL2 | rs327221 | 8 | 26536952 | G | A |
| 3966 | DPYSL2 | rs327225 | 8 | 26539107 | T | G |
| 3967 | DPYSL2 | rs17322528 | 8 | 26544163 | G | T |
| 3968 | DPYSL2 | rs327216 | 8 | 26547380 | C | T |
| 3969 | DPYSL2 | rs1867042 | 8 | 26548477 | T | G |
| 3970 | DPYSL2 | rs13277175 | 8 | 26553930 | T | G |
| 3971 | DPYSL2 | rs4733048 | 8 | 26555006 | T | C |
| 3972 | DPYSL2 | rs10088957 | 8 | 26555035 | A | G |
| 3973 | DPYSL2 | rs17055593 | 8 | 26556007 | A | C |
| 3974 | DPYSL2 | rs10093591 | 8 | 26556547 | A | G |
| 3975 | DPYSL2 | rs9644117 | 8 | 26561435 | C | T |
| 3976 | DPYSL2 | rs6992095 | 8 | 26564684 | T | C |
| 3977 | DPYSL2 | rs11863 | 8 | 26570233 | C | T |
| 3978 | DPYSL2 | rs920633 | 8 | 26570696 | C | T |
| 3979 | DPYSL2 | rs17666 | 8 | 26571375 | T | C |
| 3980 | DPYSL2 | rs7845740 | 8 | 26574668 | G | T |
| 3981 | DPYSL2 | rs7842128 | 8 | 26574697 | G | A |
| 3982 | UNC5D | rs2589344 | 8 | 35516694 | C | T |
| 3983 | UNC5D | rs7002972 | 8 | 35521243 | C | T |
| 3984 | UNC5D | rs7833633 | 8 | 35529967 | A | G |
| 3985 | UNC5D | rs6980691 | 8 | 35531940 | T | G |
| 3986 | UNC5D | rs2589338 | 8 | 35548615 | C | T |
| 3987 | UNC5D | rs2067700 | 8 | 35552625 | C | T |
| 3988 | UNC5D | rs2589746 | 8 | 35555578 | T | C |
| 3989 | UNC5D | rs2589763 | 8 | 35557697 | C | T |
| 3990 | UNC5D | rs2579903 | 8 | 35561855 | T | C |
| 3991 | UNC5D | rs7826624 | 8 | 35586829 | C | T |
| 3992 | UNC5D | rs17353731 | 8 | 35599010 | A | G |
| 3993 | UNC5D | rs4481588 | 8 | 35609723 | C | T |
| 3994 | UNC5D | rs13250548 | 8 | 35627942 | A | G |
| 3995 | UNC5D | rs7845368 | 8 | 35631936 | T | G |
| 3996 | UNC5D | rs1946745 | 8 | 35642314 | C | T |
| 3997 | UNC5D | rs1898259 | 8 | 35644423 | G | A |
| 3998 | UNC5D | rs17270035 | 8 | 35651979 | A | G |
| 3999 | UNC5D | rs17270119 | 8 | 35653680 | C | T |
| 4000 | UNC5D | rs10112630 | 8 | 35654668 | G | A |
| 4001 | UNC5D | rs16884264 | 8 | 35680464 | C | T |
| 4002 | UNC5D | rs13276125 | 8 | 35687256 | A | G |
| 4003 | UNC5D | rs7818904 | 8 | 35690874 | C | T |
| 4004 | UNC5D | rs12679845 | 8 | 35692305 | G | T |
| 4005 | UNC5D | rs7842132 | 8 | 35695902 | G | A |
| 4006 | UNC5D | rs16884338 | 8 | 35707163 | G | A |
| 4007 | UNC5D | rs16884352 | 8 | 35714222 | T | C |
| 4008 | UNC5D | rs1436613 | 8 | 35716697 | A | G |
| 4009 | UNC5D | rs12541844 | 8 | 35719250 | G | T |
| 4010 | UNC5D | rs6468336 | 8 | 35732602 | T | C |
| 4011 | UNC5D | rs16884409 | 8 | 35746059 | T | C |
| 4012 | UNC5D | rs16884418 | 8 | 35748085 | G | A |
| 4013 | UNC5D | rs7836205 | 8 | 35761484 | A | G |
| 4014 | UNC5D | rs13273386 | 8 | 35765533 | T | C |
| 4015 | UNC5D | rs9656774 | 8 | 35775439 | T | — |
| 4016 | PTK2 | rs7016497 | 8 | 141746506 | C | T |
| 4017 | PTK2 | rs10283368 | 8 | 141748999 | C | T |
| 4018 | PTK2 | rs4413752 | 8 | 141758154 | T | C |
| 4019 | PTK2 | rs10089107 | 8 | 141767633 | C | T |
| 4020 | PTK2 | rs11785444 | 8 | 141774178 | G | A |
| 4021 | PTK2 | rs4961287 | 8 | 141775502 | G | A |
| 4022 | PTK2 | rs4596627 | 8 | 141777576 | C | T |
| 4023 | PTK2 | rs10109684 | 8 | 141782956 | A | G |
| 4024 | PTK2 | rs10100025 | 8 | 141785466 | G | A |
| 4025 | PTK2 | rs11166995 | 8 | 141788433 | G | A |
| 4026 | PTK2 | rs7839832 | 8 | 141797656 | C | T |
| 4027 | PTK2 | rs1063164 | 8 | 141802191 | G | — |
| 4028 | PTK2 | rs11997161 | 8 | 141807769 | C | T |
| 4029 | PTK2 | rs6994744 | 8 | 141810050 | A | C |
| 4030 | PTK2 | rs4961289 | 8 | 141817447 | G | T |
| 4031 | PTK2 | rs10088001 | 8 | 141907542 | G | — |
| 4032 | PTK2 | rs7831543 | 8 | 141916431 | T | C |
| 4033 | PTK2 | rs4327896 | 8 | 141926504 | A | C |
| 4034 | PTK2 | rs1868280 | 8 | 141965436 | T | C |
| 4035 | PTK2 | rs1868279 | 8 | 141965559 | G | A |
| 4036 | PTK2 | rs4961237 | 8 | 141971675 | G | A |
| 4037 | PTK2 | rs10111852 | 8 | 142006723 | T | C |
| 4038 | PTK2 | rs12544802 | 8 | 142012928 | G | A |
| 4039 | PTK2 | rs13270490 | 8 | 142030361 | G | A |
| 4040 | PTK2 | rs7822300 | 8 | 142034017 | T | C |
| 4041 | PTK2 | rs1375062 | 8 | 142034963 | A | C |
| 4042 | PTK2 | rs7007003 | 8 | 142035349 | T | C |
| 4043 | PTK2 | rs4440675 | 8 | 142044639 | C | T |
| 4044 | PTK2 | rs12544404 | 8 | 142065125 | T | C |
| 4045 | PTK2 | rs1397380 | 8 | 142072236 | G | A |
| 4046 | PTK2 | rs306955 | 8 | 142082043 | G | A |
| 4047 | PTK2 | rs306953 | 8 | 142083719 | A | G |
| 4048 | SEMA4D | rs9410474 | 9 | 91202540 | C | T |
| 4049 | SEMA4D | rs7045789 | 9 | 91206736 | G | A |
| 4050 | SEMA4D | rs9410475 | 9 | 91211076 | G | A |
| 4051 | SEMA4D | rs11265799 | 9 | 91212291 | G | A |
| 4052 | SEMA4D | rs9410321 | 9 | 91220006 | T | C |
| 4053 | SEMA4D | rs4074459 | 9 | 91224758 | A | C |
| 4054 | SEMA4D | rs4877086 | 9 | 91225097 | G | A |
| 4055 | SEMA4D | rs4132699 | 9 | 91226247 | A | C |
| 4056 | SEMA4D | rs11265876 | 9 | 91229236 | T | G |
| 4057 | SEMA4D | rs10797122 | 9 | 91230185 | A | G |
| 4058 | SEMA4D | rs10512197 | 9 | 91230295 | G | A |
| 4059 | SEMA4D | rs10797126 | 9 | 91233004 | G | A |
| 4060 | SEMA4D | rs4078338 | 9 | 91237253 | C | T |
| 4061 | SEMA4D | rs4073713 | 9 | 91242078 | G | A |
| 4062 | SEMA4D | rs4075521 | 9 | 91254368 | G | A |
| 4063 | SEMA4D | rs7871577 | 9 | 91257623 | A | G |
| 4064 | SEMA4D | rs9410486 | 9 | 91259491 | A | G |
| 4065 | SEMA4D | rs11999884 | 9 | 91260211 | A | G |
| 4066 | SEMA4D | rs4360376 | 9 | 91264249 | A | C |
| 4067 | SEMA4D | rs9969727 | 9 | 91264654 | C | T |
| 4068 | SEMA4D | rs11265909 | 9 | 91267966 | T | C |
| 4069 | SEMA4D | rs17054928 | 9 | 91284116 | G | A |
| 4070 | PPP3R2 | rs7873495 | 9 | 103390245 | C | T |
| 4071 | PPP3R2 | rs4743473 | 9 | 103392395 | G | A |
| 4072 | PPP3R2 | rs1407877 | 9 | 103395276 | G | A |
| 4073 | PPP3R2 | rs3739724 | 9 | 103396260 | T | C |
| 4074 | PPP3R2 | rs7858998 | 9 | 103398856 | C | T |
| 4075 | PPP3R2 | rs6479056 | 9 | 103399082 | A | G |
| 4076 | RGS3 | rs6478011 | 9 | 115284803 | T | C |
| 4077 | RGS3 | rs3761820 | 9 | 115306525 | T | C |
| 4078 | RGS3 | rs7046427 | 9 | 115329238 | G | A |
| 4079 | RGS3 | rs10981815 | 9 | 115342858 | A | G |
| 4080 | RGS3 | rs10981818 | 9 | 115348530 | G | T |
| 4081 | RGS3 | rs4979257 | 9 | 115374598 | A | G |
| 4082 | RGS3 | rs10981835 | 9 | 115389119 | G | A |
| 4083 | RGS3 | rs4979261 | 9 | 115395236 | A | G |
| 4084 | RGS3 | rs12341266 | 9 | 115396337 | G | A |
| 4085 | RGS3 | rs17187621 | 9 | 115402559 | A | G |
| 4086 | RGS3 | rs518636 | 9 | 115402967 | C | T |
| 4087 | RGS3 | rs511561 | 9 | 115403698 | C | T |
| 4088 | ABL1 | rs2583839 | 9 | 132583760 | C | T |
| 4089 | ABL1 | rs4740359 | 9 | 132585040 | G | A |
| 4090 | ABL1 | rs10751508 | 9 | 132597937 | C | T |
| 4091 | ABL1 | rs2855160 | 9 | 132607062 | C | T |
| 4092 | ABL1 | rs2791731 | 9 | 132607692 | T | C |
| 4093 | ABL1 | rs2253084 | 9 | 132611594 | C | T |
| 4094 | ABL1 | rs4740363 | 9 | 132619005 | A | G |
| 4095 | ABL1 | rs4740366 | 9 | 132620443 | A | G |
| 4096 | ABL1 | rs2855169 | 9 | 132620722 | T | C |
| 4097 | ABL1 | rs2583836 | 9 | 132621042 | T | C |
| 4098 | ABL1 | rs4740203 | 9 | 132626629 | C | T |
| 4099 | ABL1 | rs2855171 | 9 | 132628764 | C | T |
| 4100 | ABL1 | rs4990084 | 9 | 132636413 | T | C |
| 4101 | ABL1 | rs2251403 | 9 | 132646156 | T | C |
| 4102 | ABL1 | rs2855179 | 9 | 132647102 | A | G |
| 4103 | ABL1 | rs7026988 | 9 | 132657720 | C | T |
| 4104 | ABL1 | rs10901285 | 9 | 132660695 | G | A |
| 4105 | ABL1 | rs11244147 | 9 | 132666989 | T | G |
| 4106 | ABL1 | rs10901291 | 9 | 132707702 | C | T |
| 4107 | ABL1 | rs3824400 | 9 | 132723896 | G | A |
| 4108 | ABL1 | rs11244164 | 9 | 132725937 | A | G |
| 4109 | ABL1 | rs12003046 | 9 | 132731447 | T | C |
| 4110 | ABL1 | rs3780282 | 9 | 132733475 | C | T |
| 4111 | ABL1 | rs11792273 | 9 | 132743853 | A | G |
| 4112 | ABL1 | rs2696141 | 9 | 132743920 | G | T |
| 4113 | ABL1 | rs12005009 | 9 | 132745575 | T | C |
| 4114 | ABL1 | rs3808814 | 9 | 132747606 | C | T |
| 4115 | ABL1 | rs1056209 | 9 | 132752411 | G | A |
| 4116 | ABL1 | rs2855197 | 9 | 132754227 | A | G |
| 4117 | ABL1 | rs10901296 | 9 | 132755477 | G | A |
| 4118 | EFNB1 | rs697500 | X | 67961793 | C | T |
| 4119 | EFNB1 | rs806776 | X | 67962073 | A | G |
| 4120 | EFNB1 | rs638408 | X | 67967740 | T | C |
| 4121 | EFNB1 | rs877818 | X | 67970232 | G | A |
| 4122 | PAK3 | rs4893483 | X | 110240008 | C | — |
| 4123 | PAK3 | rs5942719 | X | 110245291 | C | T |
| 4124 | PAK3 | rs2208017 | X | 110263176 | G | A |
| 4125 | PAK3 | rs12116372 | X | 110282235 | G | — |
| 4126 | PAK3 | rs10521531 | X | 110287303 | A | G |
| 4127 | PAK3 | rs9320060 | X | 110300106 | T | C |
| 4128 | PAK3 | rs7886027 | X | 110303715 | A | C |
| 4129 | PAK3 | rs16986439 | X | 110346611 | C | T |
| 4130 | PLXNB3 | rs4898439 | X | 152680827 | A | C |
| 4131 | PLXNB3 | rs762650 | X | 152698396 | G | A |
| 4132 | PLXNB3 | rs762651 | X | 152698632 | G | A |
| 4133 | PLXNA3 | rs5945431 | X | 153354910 | A | G |

TABLE 13
SNPs in Axon Guidance Pathway Genes Predicting ALS Susceptibility

| Gene (rs ID), Final | | | |
|---|---|---|---|
| Coding Scheme^{†} | Odds Ratio (95% CI)** | | P value** |
| ABLIM1 (rs11196845), d | 10.27 | (3.074-34.32) | 1.5 × 10⁻⁰⁴ |
| ABLIM2 (rs6849076), r | 0.05 | (0.01-0.25) | 2.6 × 10⁻⁰⁴ |
| ABLIM2 (rs13125250), r | 41.19 | (2.646-641.14) | 7.9 × 10⁻⁰³ |
| CDC42 (rs2056975), d | 15.45 | (3.976-60.06) | 7.7 × 10⁻⁰⁵ |
| CXCL12 (rs2297630), a | 0.14 | (0.062-0.31) | 1.5 × 10⁻⁰⁶ |
| DCC (rs1460200), r | 8.40 | (2.455-28.73) | 7.0 × 10⁻⁰⁴ |
| DCC (rs1222938), d | 7.54 | (2.801-20.28) | 6.3 × 10⁻⁰⁵ |
| DCC (rs3862675), a | 11.57 | (4.489-29.81) | 4.0 × 10⁻⁰⁷ |
| DPYSL2 (rs327221), d | 0.11 | (0.034-0.37) | 2.9 × 10⁻⁰⁴ |
| EFNA4 (rs11264306), a | 0.20 | (0.088-0.46) | 1.6 × 10⁻⁰⁴ |
| EFNA5 (rs2112912), a | 3.71 | (1.749-7.89) | 6.4 × 10⁻⁰⁴ |
| EPHA4 (rs10498114), a | 0.18 | (0.062-0.52) | 1.6 × 10⁻⁰³ |
| EPHA4 (rs10498110), a | 7.47 | (2.827-19.72) | 5.0 × 10⁻⁰⁵ |
| EPHA5 (rs7349683), r | 18.04 | (3.789-85.89) | 2.8 × 10⁻⁰⁴ |
| EPHB1 (rs16842755), r | 137.78 | (12.115-1567.04) | 7.2 × 10⁻⁰⁵ |
| EPHB1 (rs6801620), d | 0.05 | (0.014-0.18) | 4.2 × 10⁻⁰⁶ |
| EPHB1 (rs40347), d | 6.12 | (1.824-20.52) | 3.4 × 10⁻⁰³ |
| EPHB1 (rs6782829), a | 20.05 | (3.212-125.14) | 1.3 × 10⁻⁰³ |
| EPHB1 (rs9865620), d | 0.21 | (0.075-0.56) | 1.8 × 10⁻⁰³ |
| FYN (rs809193), r | 39.36 | (3.579-432.78) | 2.7 × 10⁻⁰³ |
| GSK3B (rs3108749), d | 0.27 | (0.076-0.97) | 4.5 × 10⁻⁰² |
| ITGB1 (rs2488320), a | 0.44 | (0.236-0.83) | 1.1 × 10⁻⁰² |
| LRRC4C (rs7930525), r | 0.02 | (0.003-0.12) | 3.2 × 10⁻⁰⁵ |
| LRRC4C (rs4509742), r | 37.09 | (6.442-213.52) | 5.2 × 10⁻⁰⁵ |
| NGEF (rs13383577), d | 0.11 | (0.033-0.35) | 2.0 × 10⁻⁰⁴ |
| NTN1 (rs4791334), d | 0.08 | (0.027-0.25) | 1.2 × 10⁻⁰⁵ |
| NTNG1 (rs6662274), a | 1.95 | (1.009-3.78) | 4.7 × 10⁻⁰² |
| RAC2 (rs2284037), a | 7.15 | (3.027-16.89) | 7.3 × 10⁻⁰⁶ |
| ROBO1 (rs1383407), a | 0.27 | (0.128-0.55) | 3.7 × 10⁻⁰⁴ |
| ROBO1 (rs7625555), a | 0.35 | (0.175-0.70) | 2.8 × 10⁻⁰³ |
| ROBO2 (rs6548505), d | 0.12 | (0.042-0.37) | 1.8 × 10⁻⁰⁴ |
| SEMA5A (rs465991), d | 0.03 | (0.009-0.12) | 3.2 × 10⁻⁰⁷ |
| SEMA5A (rs1512508), d | 8.86 | (2.775-28.30) | 2.3 × 10⁻⁰⁴ |
| SEMA5B (rs7432970), a | 0.13 | (0.041-0.41) | 4.4 × 10⁻⁰⁴ |
| SEMA6A (rs17139912), d | 0.03 | (0.006-0.16) | 4.6 × 10⁻⁰⁵ |
| SEMA6A (rs187710), r | 9.55 | (1.717-53.07) | 1.0 × 10⁻⁰² |
| SLIT1 (rs2636770), a | 0.09 | (0.028-0.28) | 3.3 × 10⁻⁰⁵ |
| SLIT1 (rs2817685), r | 19.95 | (3.892-102.25) | 3.3 × 10⁻⁰⁴ |
| SLIT2 (rs729918), a | 0.28 | (0.129-0.63) | 1.8 × 10⁻⁰³ |
| SLIT2 (rs1379659), r | 87.12 | (6.801-1116.02) | 6.0 × 10⁻⁰⁴ |
| SLIT3 (rs297838), a | 0.06 | (0.014-0.21) | 2.2 × 10⁻⁰⁵ |
| SLIT3 (rs17666), r | 50.29 | (8.626-293.22) | 1.3 × 10⁻⁰⁵ |
| SRGAP2 (rs2336938), d | 0.12 | (0.037-0.39) | 4.1 × 10⁻⁰⁴ |
| SRGAP3 (rs486012), r | 5.59 | (1.783-17.49) | 3.1 × 10⁻⁰³ |
| SRGAP3 (rs2664079), d | 0.06 | (0.016-0.19) | 4.5 × 10⁻⁰⁶ |
| UNC5C (rs4406027), a | 0.29 | (0.09-0.92) | 3.5 × 10⁻⁰² |
| UNC5D (rs2579903), a | 10.05 | (2.651-38.07) | 6.9 × 10⁻⁰⁴ |

**Results adjusted for age and gender.
^{†}a = log-additive, d = Mendelian dominant, r = Mendelian recessive.

### Survival Free of ALS

Of the 4,133 SNPs, 451 (10.9%) were individually associated with survival free of ALS (hazard function) using Cox proportional hazards models, as detailed in Table 12. Table 14 contains results for the final proportional hazards model produced by running SNPs through the multi-stage process to predict survival free of ALS. This model used data from 274 ALS patients (1 patient was missing data on one or more SNPs). In this case, the model had an overall p value of 1.25×10⁻⁷⁴ (95% CI 2.22×10⁻⁶⁰-1.24×10⁻⁸⁹). By contrast, the model was not significant at predicting survival (age at study) of the matched sibling controls (p=0.15). This last finding suggests that the model predicts survival free of ALS (hazard function), but not survival in general, and that the model is specific for ALS cases.

TABLE 14
SNPs in Axon Guidance Pathway Genes Predicting Survival Free of ALS.

| Gene* (rs ID), | Hazard Ratio | | | Hazard Ratio | |
|---|---|---|---|---|---|
| Final Coding Scheme^{†} | (95% CI) | P value | Interaction* | (95% CI) | P value |
| ABL1 (rs10751508), a | 1.54 (1.148-2.07) | 4.0 × 10⁻⁰³ | DPYSL5*PLXNC1 | 4.94 (2.198-11.11) | 1.1 × 10⁻⁰⁴ |
| ABLIM2 (rs4696769), r | 2.36 (1.613-3.45) | 9.6 × 10⁻⁰⁶ | SLIT1*NFAT5 | 0.15 (0.063-0.35) | 1.6 × 10⁻⁰⁵ |
| ABLIM2 (rs11724027), d | 0.30 (0.168-0.54) | 6.7 × 10⁻⁰⁵ | ROBO1₂*DPYSL2₂ | 8.18 (2.464-27.13) | 6.0 × 10⁻⁰⁴ |
| CFL1 (rs635375), d | 0.57 (0.423-0.77) | 2.7 × 10⁻⁰⁴ | | | |
| DCC (rs7233856), r | 27.83 (9.791-79.08) | 4.4 × 10⁻¹⁰ | | | |
| DCC (rs4940206), d | 2.97 (2.094-4.22) | 1.1 × 10⁻⁰⁹ | | | |
| DCC (rs1943107), r | 12.25 (3.323-45.13) | 1.7 × 10⁻⁰⁴ | | | |
| DCC (rs1431730), d | 0.61 (0.411-0.91) | 1.4 × 10⁻⁰² | | | |
| DCC (rs9966348), a | 2.44 (1.854-3.21) | 2.0 × 10−¹⁰ | | | |
| DCC (rs727514), r | 7.82 (3.181-19.23) | 7.4 × 10⁻⁰⁶ | | | |
| DPYSL2₁ (rs4733048), r | 4.34 (1.488-12.63) | 7.2 × 10⁻⁰³ | | | |
| DPYSL2₂ (rs6992095), r | 5.00 (2.188-11.44) | 1.4 × 10⁻⁰⁴ | | | |
| DPYSL5 (rs1371614), r | 2.62 (1.132-6.07) | 2.4 × 10⁻⁰² | | | |
| EFNA1 (rs9297), d | 0.39 (0.278-0.54) | 1.2 × 10⁻⁰⁸ | | | |
| EFNA5 (rs152555), a | 1.65 (1.221-2.22) | 1.1 × 10⁻⁰³ | | | |
| EFNA5 (rs26249), d | 3.81 (2.426-5.98) | 6.3 × 10⁻⁰⁹ | | | |
| EPHA4 (rs10498120), r | 7.31 (3.233-16.54) | 1.8 × 10⁻⁰⁶ | | | |
| EPHB1 (rs9871429), r | 0.48 (0.339-0.69) | 5.7 × 10⁻⁰⁵ | | | |
| EPHB1 (rs16842240), r | 0.04 (0.007-0.17) | 3.1 × 10⁻⁰⁵ | | | |
| EPHB1 (rs972234), a | 1.80 (1.442-2.25) | 2.1 × 10⁻⁰⁷ | | | |
| EPHB2 (rs294218), a | 2.35 (1.853-2.97) | 1.5 × 10⁻¹² | | | |
| FES (rs1029420), r | 0.25 (0.166-0.37) | 2.2 × 10⁻¹¹ | | | |
| LRRC4C (rs7106026), r | 4.40 (1.127-17.17) | 3.3 × 10⁻⁰² | | | |
| NFAT5 (rs12447326), a | 1.81 (1.438-2.29) | 5.0 × 10⁻⁰⁷ | | | |
| NFATC1 (rs8090312), d | 0.53 (0.391-0.73) | 7.6 × 10⁻⁰⁵ | | | |
| NFATC2 (rs6126251), a | 2.02 (1.554-2.62) | 1.3 × 10⁻⁰⁷ | | | |
| NGEF (rs2004203), a | 1.71 (1.348-2.18) | 1.1 × 10⁻⁰⁵ | | | |
| NGEF (rs6707071), r | 1.78 (1.243-2.55) | 1.7 × 10⁻⁰³ | | | |
| NGEF (rs709937), r | 2.84 (1.653-4.89) | 1.6 × 10⁻⁰⁴ | | | |
| NRP1 (rs11593943), r | 2.08 (1.379-3.15) | 4.9 × 10⁻⁰⁴ | | | |
| NTN1 (rs10852910), r | 2.94 (1.566-5.53) | 8.0 × 10⁻⁰⁴ | | | |
| NTN1 (rs4791798), d | 0.32 (0.234-0.43) | 3.7 × 10⁻¹³ | | | |
| NTNG1 (rs10881468), r | 2.21 (1.393-3.52) | 7.7 × 10⁻⁰⁴ | | | |
| PAK2 (rs6583176), d | 2.19 (1.593-3.00) | 1.3 × 10⁻⁰⁶ | | | |
| PAK7 (rs2423443), r | 3.58 (2.396-5.35) | 4.9 × 10⁻¹⁰ | | | |
| PLXNA2 (rs12094094), r | 3.98 (2.201-7.20) | 4.9 × 10⁻⁰⁶ | | | |
| PLXNA2 (rs493194), r | 26.18 (8.705-78.74) | 6.2 × 10⁻⁰⁹ | | | |
| PLXNC1 (rs10777588), a | 1.33 (1.068-1.66) | 1.1 × 10⁻⁰² | | | |
| ROBO1₁ (rs162871), a | 2.52 (1.743-3.64) | 8.8 × 10⁻⁰⁷ | | | |
| ROBO1₂ (rs12635896), a | 0.56 (0.395-0.78) | 8.0 × 10⁻⁰⁴ | | | |
| RRAS (rs1865077), d | 0.61 (0.464-0.80) | 4.6 × 10⁻⁰⁴ | | | |
| SEMA3D (rs1029563), r | 3.92 (2.092-7.35) | 2.0 × 10⁻⁰⁵ | | | |
| SEMA3D (rs2286185), a | 0.58 (0.466-0.73) | 2.6 × 10⁻⁰⁶ | | | |
| SEMA3E (rs2535372), r | 4.78 (2.421-9.45) | 6.7 × 10⁻⁰⁶ | | | |
| SEMA5A (rs3798004), d | 2.09 (1.134-3.86) | 1.8 × 10⁻⁰² | | | |
| SEMA5B (rs4677983), a | 1.64 (1.205-2.23) | 1.6 × 10⁻⁰³ | | | |
| SLIT1 (rs2817685), r | 0.72 (0.400-1.29) | 2.7 × 10⁻⁰¹ | | | |
| SLIT2 (rs17612037), d | 0.42 (0.308-0.58) | 1.1 × 10⁻⁰⁷ | | | |
| SLIT2 (rs6447955), d | 0.39 (0.272-0.56) | 2.1 × 10⁻⁰⁷ | | | |
| SLIT3 (rs7712111), r | 1.81 (1.164-2.81) | 8.4 × 10⁻⁰³ | | | |
| SLIT3 (rs10071654), r | 2.94 (2.012-4.30) | 2.5 × 10⁻⁰⁸ | | | |
| UNC5C (rs4406027), d | 1.71 (1.165-2.50) | 6.1 × 10⁻⁰³ | | | |

*Subscript is used to identify individual SNPs in interactions.
^{†}a = log-additive, d = Mendelian dominant, r = Mendelian recessive.

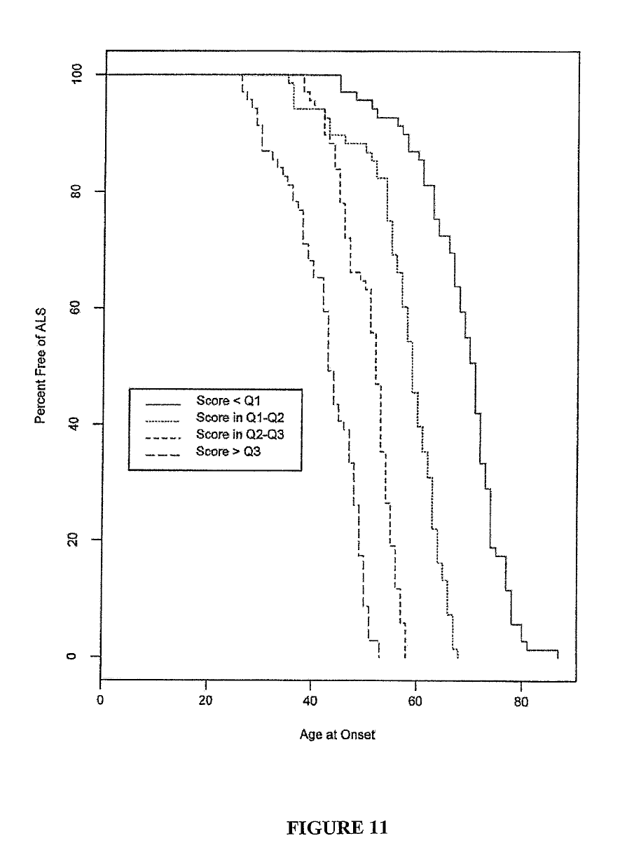

FIG. 11 shows a Kaplan-Meier plot to describe the results of the model. The groups were formed by calculating a risk score for each ALS patient using the equation from the proportional hazards model, then categorizing the score at the 25^{th} (Q1), 50^{th} (Q2), and 75^{th} (Q3) percentiles. The survival curves separated nicely right from the earliest ages of onset. By age 50, only 9% of ALS patients in the predicted highest risk group were still free of ALS, whereas 96% of ALS patients in the predicted lowest risk group were free of ALS. By age 55, none of the ALS patients in the predicted highest risk group were still free of ALS, whereas 93% of ALS patients in the predicted lowest risk group were free of ALS. The median ages at onset for each group, from lowest risk group to highest risk group, were 71, 59, 52, and 43, a difference in survival free of ALS of 28 years from lowest to highest. The concordance for this model was 0.86. The hazard ratios (95% CIs) for the four groups, from lowest to highest risk, were 1 (reference), 7.35 (4.50-12.00), 37.29 (20.49-67.87), and 149.80 (77.21-290.63).

### Age at Onset of ALS

Of the 4,133 SNPs, 487 (11.8%) were individually associated with age at onset of ALS using linear regression models, as detailed in Table 12. Table 15 contains results for the final model produced by running SNPs through the multi-stage process to predict ALS age at onset. This model used data from 272 ALS patients (3 patients were missing data on one or more SNPs). In this case, the model had an overall p value of 9.14×10⁻⁷⁶ (95% CI 8.99×10⁻⁵⁸-3.19×10⁻⁹²). By contrast, the set of SNPs was not significant at predicting age at study of the matched sibling controls (p=0.55). This last finding suggests that the model predicts age at onset of ALS, not age at the time of the study, and that the model is specific for ALS cases.

TABLE 15
SNPs in Axon Guidance Pathway Genes Predicting Age at Onset of ALS.

| | Regression | | | Regression | |
|---|---|---|---|---|---|
| Gene (rs ID), | coefficient | | | coefficient | |
| Final Coding Scheme^{†} | (standard error) | P value | Interaction* | (standard error) | P value |
| ABL1 (rs4740366), a | −3.29 (0.70) | 5.1 × 10⁻⁰⁶ | ROBO1₁*NTN1₂ | 11.28 (2.60) | 2.2 × 10⁻⁰⁵ |
| ABLIM1 (rs11196865), a | 2.12 (0.76) | 5.7 × 10⁻⁰³ | PAK7₁*UNC5C | −7.37 (1.87) | 1.1 × 10⁻⁰⁴ |
| CDC42 (rs2473323), a | 3.70 (0.93) | 9.7 × 10⁻⁰⁵ | UNC5C*RAC2 | −10.86 (2.80) | 1.4 × 10⁻⁰⁴ |
| CXCL12 (rs2839692), r | 6.71 (1.77) | 1.9 × 10⁻⁰⁴ | NGEF₂*PAK7₂ | −6.59 (1.86) | 4.7 × 10⁻⁰⁴ |
| DCC (rs4940206), d | −4.82 (0.77) | 2.4 × 10⁻⁰⁹ | NGEF₁*EFNA4 | −5.28 (2.06) | 1.1 × 10⁻⁰² |
| DCC (rs7233856), r | −10.65 (2.35) | 9.5 × 10⁻⁰⁶ | EPHB1₁*EPHB1₂ | −3.65 (1.30) | 5.5 × 10⁻⁰³ |
| DCC (rs9966348), a | −3.39 (0.64) | 2.4 × 10⁻⁰⁷ | NTN1₁*SRGAP3₂ | 2.80 (1.04) | 7.4 × 10⁻⁰³ |
| EFNA4 (rs11264306), r | −0.62 (1.07) | 5.6 × 10⁻⁰¹ | | | |
| EFNA5 (rs26249), d | −3.87 (0.99) | 1.3 × 10⁻⁰⁴ | | | |
| EFNA5 (rs152608), a | 1.30 (0.59) | 2.8 × 10⁻⁰² | | | |
| EFNB1 (rs877818), d | 2.70 (0.78) | 6.6 × 10⁻⁰⁴ | | | |
| EPHA7 (rs345716), a | 5.19 (1.15) | 1.1 × 10⁻⁰⁵ | | | |
| EPHB1₁ (rs36041), r | −0.64 (1.38) | 6.4 × 10⁻⁰¹ | | | |
| EPHB1₂ (rs9815290), a | −2.11 (0.54) | 1.4 × 10⁻⁰⁴ | | | |
| EPHB1₃ (rs3772638), r | 4.75 (1.38) | 7.2 × 10⁻⁰⁴ | | | |
| FES (rs1029420), r | 6.64 (0.87) | 1.0 × 10⁻¹² | | | |
| KRAS (rs10842514), r | −2.75 (0.90) | 2.4 × 10⁻⁰³ | | | |
| LRRC4C (rs4076390), r | 3.67 (1.01) | 3.7 × 10⁻⁰⁴ | | | |
| LRRC4C (rs16934480), r | 5.28 (2.23) | 1.9 × 10⁻⁰² | | | |
| LRRC4C (rs17477151), d | 3.76 (1.05) | 4.0 × 10⁻⁰⁴ | | | |
| NGEF₁ (rs6707071), r | −2.11 (0.96) | 2.9 × 10⁻⁰² | | | |
| NGEF₂ (rs2004203), a | −0.72 (0.56) | 2.0 × 10⁻⁰¹ | | | |
| NTN1₁ (rs4791334), a | −2.97 (0.62) | 2.9 × 10⁻⁰⁶ | | | |
| NTN1₂ (rs10852910), r | −7.58 (1.78) | 3.1 × 10⁻⁰⁵ | | | |
| NTN1₃ (rs9897341), a | −2.33 (0.61) | 1.7 × 10⁻⁰⁴ | | | |
| NTN4 (rs7310388), r | 3.19 (0.87) | 3.3 × 10⁻⁰⁴ | | | |
| NTN4 (rs4129599), r | −5.81 (2.14) | 7.0 × 10⁻⁰³ | | | |
| PAK7₁ (rs2254000), r | −2.44 (1.34) | 7.0 × 10⁻⁰² | | | |
| PAK7₂ (rs6056725), d | 0.27 (1.69) | 8.7 × 10⁻⁰¹ | | | |
| PLXNA2 (rs12094094), r | −5.84 (1.42) | 5.9 × 10⁻⁰⁵ | | | |
| PPP3CC (rs17733133), a | −6.13 (0.81) | 1.0 × 10⁻¹² | | | |
| RAC2 (rs972430), r | 11.19 (2.30) | 2.2 × 10⁻⁰⁶ | | | |
| RGS3 (rs3761820), r | 14.13 (2.29) | 3.4 × 10⁻⁰⁹ | | | |
| ROBO1₁ (rs1865862), a | 7.62 (1.33) | 3.0 × 10⁻⁰⁸ | | | |
| ROBO1₂ (rs2608021), a | −7.37 (1.40) | 3.2 × 10⁻⁰⁷ | | | |
| ROBO1₃ (rs4680919), r | 6.87 (2.07) | 1.0 × 10⁻⁰³ | | | |
| ROBO2 (rs9809640), d | −3.68 (0.74) | 1.2 × 10⁻⁰⁶ | | | |
| RRAS (rs1865077), d | 3.53 (0.69) | 6.5 × 10⁻⁰⁷ | | | |
| SEMA3E (rs2535372), a | −2.87 (0.60) | 3.3 × 10⁻⁰⁶ | | | |
| SLIT2 (rs729918), a | −1.87 (0.55) | 7.3 × 10⁻⁰⁴ | | | |
| SLIT2 (rs16869665), a | −3.58 (0.97) | 2.9 × 10⁻⁰⁴ | | | |
| SLIT3 (rs12515660), a | −4.56 (1.14) | 8.3 × 10⁻⁰⁵ | | | |
| SLIT3 (rs11134544), r | −3.45 (1.04) | 1.1 × 10⁻⁰³ | | | |
| SRGAP3₁ (rs2675181), a | −1.71 (0.54) | 1.7 × 10⁻⁰³ | | | |
| SRGAP3₂ (rs13088083), d | 2.27 (1.03) | 2.8 × 10⁻⁰² | | | |
| UNC5C (rs7679033), d | 5.64 (0.83) | 8.5 × 10⁻¹¹ | | | |
| UNC5D (rs10112630), r | 2.62 (0.83) | 1.9 × 10⁻⁰³ | | | |

*Subscript is used to identify individual SNPs in interactions.
^{†}a = log-additive, d = Mendelian dominant, r = Mendelian recessive.

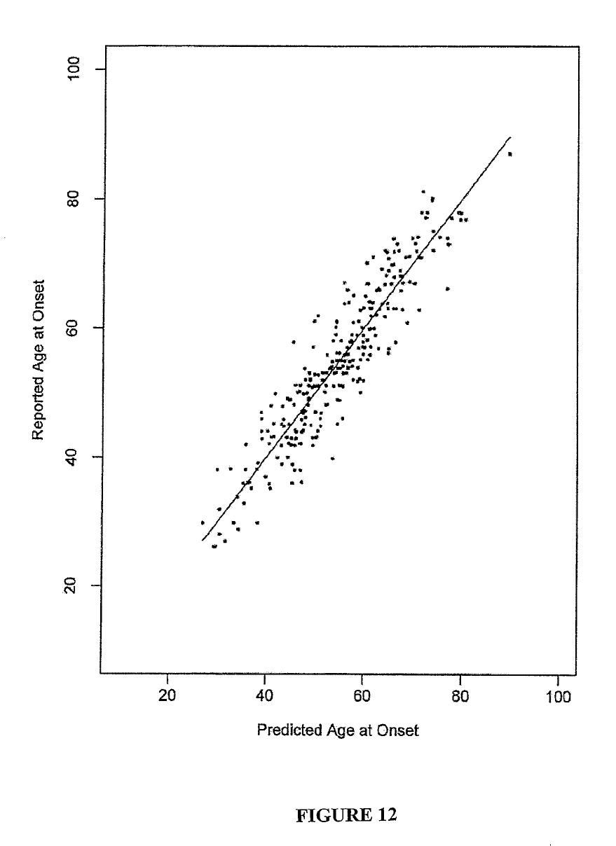

FIG. 12 shows a plot of predicted age at onset vs. reported age at onset to summarize the results of the model. The plot showed a nice elliptical pattern, reflecting the model R² of 0.86 (95% CI 0.83-0.88). The model explained about 86% of the variability in age at onset of ALS.

Other combinations of SNPs from the axon guidance pathway also performed quite well in predicting ALS susceptibility, survival free of ALS, and age at onset of ALS. Although the models provided herein provided good fits to the data, the results do not preclude other combinations of axon guidance pathway SNPs as significant predictors of ALS. The SNPs in the final models selected showed no significant linkage disequilibrium in unrelated controls.

### Example 4

### Axon Guidance Pathway Genes and PD

Examples 1 and 4 are similar but for Example 1, the analysis of the secondary whole genome association dataset is restricted to the 45 genes that were represented by SNPs in the final predictive models of PD in the primary dataset. In contrast, Example 4 considers the secondary whole genome association dataset only, without restriction to the 45 genes of Example 1. In other words, Example 4 allows consideration of all 128 brain expressed axon guidance pathway genes within the secondary whole genome association dataset.

This study employed a genomic pathway approach to determine whether polymorphism in the axon guidance pathway predisposed to PD. For example, we employed bioinformatic methods to mine an available whole-genome association dataset for SNPs that were within brain-expressed, axon guidance pathway genes. We then employed statistical methods to construct models of axon guidance pathway SNPs that predicted three outcomes: PD susceptibility, survival free of PD, and age at onset of PD. The primary whole-genome association study dataset employed by this study included 269 PD cases and 267 unrelated controls. The median age at onset of PD among the cases was 64 years (range 13-84). The bioinformatic methods identified 128 brain-expressed axon guidance pathway genes and the SNP dataset included 3,095 SNPs within 122 of those genes.

### PD Susceptibility

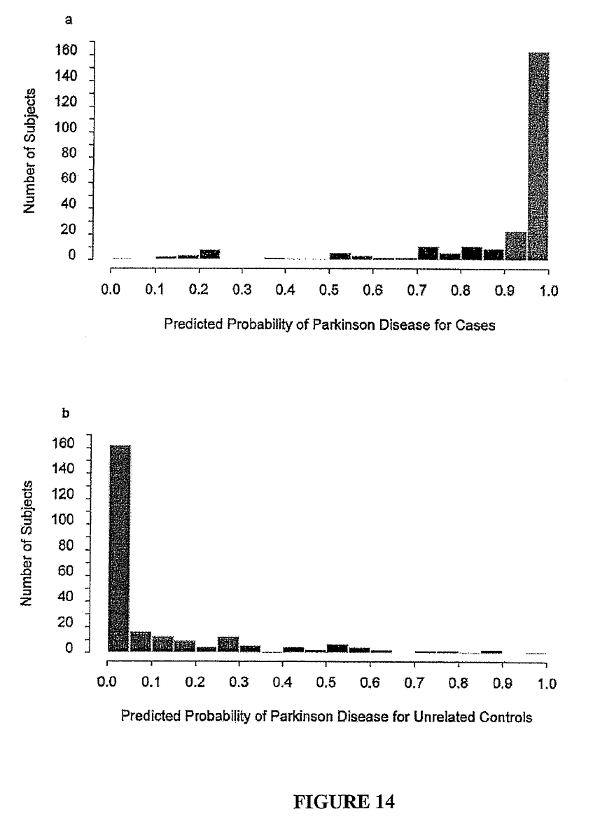

Of the 3,095 SNPs within brain-expressed genes of the axon-guidance pathway, 295 SNPs (9.5%) were individually associated with susceptibility to PD, as detailed in Table 16. Table 17 contains results for the final model produced by running SNPs through the multi-stage process to predict PD susceptibility. This model used data from 516 unmatched PD patients and unrelated controls (20 subjects were missing data on one or more SNPs). The odds ratios (95% CIs) for the groups defined by predicted PD probability of <0.25, 0.25-0.50, 0.50-0.75, and >0.75 were as follows: 1 (reference), 1.85 (0.58-5.92), 19.03 (8.56-42.30), and 391.82 (157.94-972.06), respectively. Since we were interested in the significance of the pathway, rather than individual SNPs, the p value for the overall model was of primary importance. In this case, the model had an overall p value of 8.10×10⁻⁷¹ (95% CI 2.34×10⁻⁶⁴-1.67×10⁻⁷⁶). This model significantly predicted whether or not an individual was a case or an unrelated control. The predicted probabilities of PD were very high (towards 1) for most of the cases, and very low (towards 0) for most of the unrelated controls (FIG. 14). Indeed, 72% of the cases had predicted probabilities above 0.9, and 69% of unrelated controls had predicted probabilities below 0.1. However, as shown by FIG. 14, the model did not completely distinguish the two groups; some cases had low predicted probabilities, and some controls had high predicted probabilities. The concordance for the model was about 0.98, indicating excellent agreement between predicted and observed case/control status.

TABLE 16
Axon-guidance SNPs.

| ID | Gene Name | rsID | Chromosome | Position | Reference Allele | Alternate Allele |
|---|---|---|---|---|---|---|
| 1 | ABL1 | rs2583839 | 9 | 132583759 | C | T |
| 2 | ABL1 | rs2855160 | 9 | 132607061 | C | T |
| 3 | ABL1 | rs2791731 | 9 | 132607691 | T | C |
| 4 | ABL1 | rs2253084 | 9 | 132611593 | C | T |
| 5 | ABL1 | rs4740363 | 9 | 132619004 | A | G |
| 6 | ABL1 | rs4740366 | 9 | 132620442 | A | G |
| 7 | ABL1 | rs2855169 | 9 | 132620721 | T | C |
| 8 | ABL1 | rs4740203 | 9 | 132626628 | C | T |
| 9 | ABL1 | rs4990084 | 9 | 132636412 | T | C |
| 10 | ABL1 | rs7026988 | 9 | 132657719 | C | T |
| 11 | ABL1 | rs2314342 | 9 | 132697182 | T | C |
| 12 | ABL1 | rs10901291 | 9 | 132707701 | C | T |
| 13 | ABL1 | rs3780284 | 9 | 132723781 | T | C |
| 14 | ABL1 | rs3824400 | 9 | 132723895 | G | A |
| 15 | ABL1 | rs12003046 | 9 | 132731446 | T | C |
| 16 | ABL1 | rs7848941 | 9 | 132734492 | G | A |
| 17 | ABL1 | rs2696141 | 9 | 132743919 | G | T |
| 18 | ABL1 | rs3808814 | 9 | 132747605 | C | T |
| 19 | ABL1 | rs11991 | 9 | 132752378 | T | C |
| 20 | ABL1 | rs1056209 | 9 | 132752410 | G | A |
| 21 | ABL1 | rs7457 | 9 | 132752646 | C | T |
| 22 | ABLIM1 | rs6646 | 10 | 116181163 | C | T |
| 23 | ABLIM1 | rs3750820 | 10 | 116182615 | T | C |
| 24 | ABLIM1 | rs10787530 | 10 | 116183906 | G | A |
| 25 | ABLIM1 | rs3802734 | 10 | 116184199 | G | A |
| 26 | ABLIM1 | rs4751906 | 10 | 116184769 | C | G |
| 27 | ABLIM1 | rs7919465 | 10 | 116185799 | T | — |
| 28 | ABLIM1 | rs941853 | 10 | 116189164 | G | A |
| 29 | ABLIM1 | rs10160093 | 10 | 116193963 | A | G |
| 30 | ABLIM1 | rs727532 | 10 | 116194765 | A | G |
| 31 | ABLIM1 | rs7080135 | 10 | 116204105 | A | C |
| 32 | ABLIM1 | rs7920878 | 10 | 116212738 | G | A |
| 33 | ABLIM1 | rs1535386 | 10 | 116223223 | A | G |
| 34 | ABLIM1 | rs10510005 | 10 | 116229074 | C | T |
| 35 | ABLIM1 | rs2145896 | 10 | 116232257 | G | T |
| 36 | ABLIM1 | rs2475224 | 10 | 116233841 | G | A |
| 37 | ABLIM1 | rs1080709 | 10 | 116235946 | A | G |
| 38 | ABLIM1 | rs2475228 | 10 | 116242854 | A | G |
| 39 | ABLIM1 | rs17717312 | 10 | 116247894 | G | A |
| 40 | ABLIM1 | rs2475230 | 10 | 116249919 | A | G |
| 41 | ABLIM1 | rs10510006 | 10 | 116250616 | A | C |
| 42 | ABLIM1 | rs2475233 | 10 | 116262975 | C | T |
| 43 | ABLIM1 | rs2497707 | 10 | 116267514 | G | A |
| 44 | ABLIM1 | rs2497708 | 10 | 116271539 | C | A |
| 45 | ABLIM1 | rs2497714 | 10 | 116282550 | G | A |
| 46 | ABLIM1 | rs10885575 | 10 | 116287286 | A | G |
| 47 | ABLIM1 | rs883768 | 10 | 116290947 | G | A |
| 48 | ABLIM1 | rs10885579 | 10 | 116294947 | T | C |
| 49 | ABLIM1 | rs10749170 | 10 | 116302099 | T | C |
| 50 | ABLIM1 | rs2483548 | 10 | 116309005 | T | C |
| 51 | ABLIM1 | rs7923449 | 10 | 116313709 | G | A |
| 52 | ABLIM1 | rs10885583 | 10 | 116320762 | T | C |
| 53 | ABLIM1 | rs2483567 | 10 | 116322759 | G | A |
| 54 | ABLIM1 | rs985273 | 10 | 116325235 | C | T |
| 55 | ABLIM1 | rs3808949 | 10 | 116326281 | A | C |
| 56 | ABLIM1 | rs2483578 | 10 | 116336213 | T | C |
| 57 | ABLIM1 | rs2483580 | 10 | 116337327 | G | A |
| 58 | ABLIM1 | rs1413195 | 10 | 116342036 | G | A |
| 59 | ABLIM1 | rs2483583 | 10 | 116344185 | T | G |
| 60 | ABLIM1 | rs2253338 | 10 | 116347519 | A | G |
| 61 | ABLIM1 | rs2277265 | 10 | 116351829 | T | C |
| 62 | ABLIM1 | rs2497665 | 10 | 116352489 | G | A |
| 63 | ABLIM1 | rs7094673 | 10 | 116352824 | G | A |
| 64 | ABLIM1 | rs10787540 | 10 | 116354413 | A | G |
| 65 | ABLIM1 | rs2497666 | 10 | 116355030 | A | G |
| 66 | ABLIM1 | rs1109267 | 10 | 116395337 | G | A |
| 67 | ABLIM1 | rs11196826 | 10 | 116397395 | C | T |
| 68 | ABLIM1 | rs4752071 | 10 | 116398626 | G | T |
| 69 | ABLIM1 | rs10885591 | 10 | 116399902 | C | A |
| 70 | ABLIM1 | rs12571587 | 10 | 116405587 | C | T |
| 71 | ABLIM1 | rs12570718 | 10 | 116406948 | C | T |
| 72 | ABLIM1 | rs1333682 | 10 | 116407536 | T | C |
| 73 | ABLIM1 | rs11196843 | 10 | 116408438 | C | T |
| 74 | ABLIM1 | rs4751639 | 10 | 116424799 | T | C |
| 75 | ABLIM1 | rs11196869 | 10 | 116432607 | G | A |
| 76 | ABLIM1 | rs11196871 | 10 | 116433025 | G | A |
| 77 | ABLIM1 | rs10885596 | 10 | 116434345 | C | T |
| 78 | ABLIM2 | rs4696709 | 4 | 8017711 | G | A |
| 79 | ABLIM2 | rs3749597 | 4 | 8018142 | C | G |
| 80 | ABLIM2 | rs4696886 | 4 | 8018582 | C | T |
| 81 | ABLIM2 | rs2292735 | 4 | 8018774 | G | A |
| 82 | ABLIM2 | rs10008860 | 4 | 8020493 | G | A |
| 83 | ABLIM2 | rs1317285 | 4 | 8031197 | C | A |
| 84 | ABLIM2 | rs1878323 | 4 | 8031624 | G | A |
| 85 | ABLIM2 | rs1574393 | 4 | 8035117 | C | T |
| 86 | ABLIM2 | rs7686189 | 4 | 8036523 | C | T |
| 87 | ABLIM2 | rs7677869 | 4 | 8037759 | C | T |
| 88 | ABLIM2 | rs4696726 | 4 | 8043531 | A | G |
| 89 | ABLIM2 | rs3814062 | 4 | 8052414 | T | C |
| 90 | ABLIM2 | rs13125250 | 4 | 8061011 | T | C |
| 91 | ABLIM2 | rs1320076 | 4 | 8062531 | C | T |
| 92 | ABLIM2 | rs1317368 | 4 | 8062595 | T | G |
| 93 | ABLIM2 | rs4696729 | 4 | 8066320 | C | T |
| 94 | ABLIM2 | rs4696666 | 4 | 8070346 | G | A |
| 95 | ABLIM2 | rs11726230 | 4 | 8077137 | G | T |
| 96 | ABLIM2 | rs2385904 | 4 | 8082370 | G | A |
| 97 | ABLIM2 | rs4696734 | 4 | 8086001 | C | A |
| 98 | ABLIM2 | rs1878324 | 4 | 8087955 | C | T |
| 99 | ABLIM2 | rs6852834 | 4 | 8096368 | A | G |
| 100 | ABLIM2 | rs2385916 | 4 | 8098872 | T | C |
| 101 | ABLIM2 | rs6849076 | 4 | 8100612 | A | G |
| 102 | ABLIM2 | rs1320062 | 4 | 8100796 | G | A |
| 103 | ABLIM2 | rs1574414 | 4 | 8100955 | G | T |
| 104 | ABLIM2 | rs10222845 | 4 | 8105371 | G | A |
| 105 | ABLIM2 | rs6834025 | 4 | 8111181 | A | T |
| 106 | ABLIM2 | rs2076769 | 4 | 8112118 | G | A |
| 107 | ABLIM2 | rs4696669 | 4 | 8113101 | A | G |
| 108 | ABLIM2 | rs16841624 | 4 | 8113729 | C | T |
| 109 | ABLIM2 | rs6845573 | 4 | 8114400 | C | T |
| 110 | ABLIM2 | rs17178569 | 4 | 8115660 | G | A |
| 111 | ABLIM2 | rs6829649 | 4 | 8121692 | T | G |
| 112 | ABLIM2 | rs6842276 | 4 | 8126033 | C | T |
| 113 | ABLIM2 | rs6826670 | 4 | 8131472 | T | G |
| 114 | ABLIM2 | rs6447849 | 4 | 8135459 | C | T |
| 115 | ABLIM2 | rs2292739 | 4 | 8140497 | C | T |
| 116 | ABLIM2 | rs1316348 | 4 | 8150567 | G | C |
| 117 | ABLIM2 | rs1316347 | 4 | 8150720 | G | A |
| 118 | ABLIM2 | rs10938688 | 4 | 8153531 | C | T |
| 119 | ABLIM2 | rs4696758 | 4 | 8157148 | A | G |
| 120 | ABLIM2 | rs1878519 | 4 | 8161681 | C | T |
| 121 | ABLIM2 | rs1105616 | 4 | 8162016 | T | G |
| 122 | ABLIM2 | rs2138826 | 4 | 8167101 | G | A |
| 123 | ABLIM2 | rs1517525 | 4 | 8170836 | C | T |
| 124 | ABLIM2 | rs4696763 | 4 | 8171565 | A | G |
| 125 | ABLIM2 | rs13144749 | 4 | 8172404 | G | A |
| 126 | ABLIM2 | rs4696765 | 4 | 8176687 | T | C |
| 127 | ABLIM2 | rs1104756 | 4 | 8181757 | A | C |
| 128 | ABLIM2 | rs4422405 | 4 | 8190857 | A | G |
| 129 | ABLIM2 | rs4696769 | 4 | 8199058 | G | A |
| 130 | ABLIM2 | rs12506374 | 4 | 8205433 | T | C |
| 131 | ABLIM2 | rs6816775 | 4 | 8207881 | T | G |
| 132 | ABLIM2 | rs7660199 | 4 | 8208609 | A | G |
| 133 | ABLIM2 | rs1533508 | 4 | 8209251 | A | G |
| 134 | ABLIM3 | rs4579247 | 5 | 148500405 | C | T |
| 135 | ABLIM3 | rs4147470 | 5 | 148508299 | T | C |
| 136 | ABLIM3 | rs4546368 | 5 | 148519292 | T | C |
| 137 | ABLIM3 | rs9325132 | 5 | 148528199 | C | T |
| 138 | ABLIM3 | rs9325134 | 5 | 148531357 | A | G |
| 139 | ABLIM3 | rs4362930 | 5 | 148540101 | T | C |
| 140 | ABLIM3 | rs6887628 | 5 | 148543173 | C | A |
| 141 | ABLIM3 | rs7720260 | 5 | 148556445 | T | C |
| 142 | ABLIM3 | rs4705331 | 5 | 148556889 | A | G |
| 143 | ABLIM3 | rs4705332 | 5 | 148557169 | T | C |
| 144 | ABLIM3 | rs4277904 | 5 | 148559329 | A | G |
| 145 | ABLIM3 | rs2918281 | 5 | 148570615 | A | G |
| 146 | ABLIM3 | rs2918268 | 5 | 148586948 | T | C |
| 147 | ABLIM3 | rs1465850 | 5 | 148591399 | G | T |
| 148 | ABLIM3 | rs998583 | 5 | 148592811 | C | A |
| 149 | ABLIM3 | rs10073851 | 5 | 148603409 | G | A |
| 150 | ABLIM3 | rs9325136 | 5 | 148607444 | A | G |
| 151 | ABLIM3 | rs2304609 | 5 | 148607695 | C | T |
| 152 | ABLIM3 | rs2289285 | 5 | 148611444 | C | T |
| 153 | ABLIM3 | rs1438688 | 5 | 148619254 | C | T |
| 154 | ARHGEF12 | rs3809059 | 11 | 119711372 | A | T |
| 155 | ARHGEF12 | rs4938800 | 11 | 119714595 | G | A |
| 156 | ARHGEF12 | rs7932658 | 11 | 119716421 | G | A |
| 157 | ARHGEF12 | rs632137 | 11 | 119752242 | G | A |
| 158 | ARHGEF12 | rs1892894 | 11 | 119759893 | C | T |
| 159 | ARHGEF12 | rs622373 | 11 | 119762054 | T | C |
| 160 | ARHGEF12 | rs6589812 | 11 | 119783568 | T | C |
| 161 | ARHGEF12 | rs11217861 | 11 | 119793269 | C | T |
| 162 | ARHGEF12 | rs6589813 | 11 | 119798699 | A | G |
| 163 | ARHGEF12 | rs723937 | 11 | 119817955 | C | T |
| 164 | ARHGEF12 | rs766894 | 11 | 119834067 | A | G |
| 165 | ARHGEF12 | rs673974 | 11 | 119848013 | G | A |
| 166 | ARHGEF12 | rs10790383 | 11 | 119849735 | C | T |
| 167 | ARHGEF12 | rs11217881 | 11 | 119849760 | A | G |
| 168 | ARHGEF12 | rs11217883 | 11 | 119850213 | A | G |
| 169 | ARHGEF12 | rs2276035 | 11 | 119851569 | C | T |
| 170 | ARHGEF12 | rs503473 | 11 | 119860439 | A | G |
| 171 | ARHGEF12 | rs11217889 | 11 | 119861526 | C | G |
| 172 | CDC42 | rs2473323 | 1 | 22261913 | A | C |
| 173 | CDC42 | rs2473322 | 1 | 22262658 | A | C |
| 174 | CDC42 | rs2056975 | 1 | 22281113 | C | T |
| 175 | CDC42 | rs2056974 | 1 | 22281680 | T | A |
| 176 | CDC42 | rs2268177 | 1 | 22287996 | A | T |
| 177 | CDK5 | rs2069459 | 7 | 150382522 | G | T |
| 178 | CDK5 | rs1549760 | 7 | 150386771 | C | T |
| 179 | CFL1 | rs635375 | 11 | 65378570 | A | G |
| 180 | CFL1 | rs665306 | 11 | 65380600 | C | T |
| 181 | CFL2 | rs10132688 | 14 | 34254898 | T | C |
| 182 | CHP | rs6492995 | 15 | 39317003 | T | C |
| 183 | CHP | rs2164996 | 15 | 39323915 | G | A |
| 184 | CHP | rs6492997 | 15 | 39328462 | G | A |
| 185 | CHP | rs6492998 | 15 | 39333922 | A | G |
| 186 | CXCL12 | rs266089 | 10 | 44189432 | G | A |
| 187 | CXCL12 | rs197452 | 10 | 44190245 | C | T |
| 188 | CXCL12 | rs266087 | 10 | 44191067 | G | A |
| 189 | CXCL12 | rs2297630 | 10 | 44191553 | G | A |
| 190 | CXCL12 | rs2839695 | 10 | 44193854 | T | C |
| 191 | CXCL12 | rs4948878 | 10 | 44194826 | A | G |
| 192 | CXCL12 | rs2839692 | 10 | 44194872 | T | C |
| 193 | CXCL12 | rs10793537 | 10 | 44196585 | G | C |
| 194 | CXCL12 | rs10793538 | 10 | 44196591 | A | T |
| 195 | CXCL12 | rs3780891 | 10 | 44198718 | G | A |
| 196 | DCC | rs2256 | 18 | 48128801 | C | A |
| 197 | DCC | rs2195781 | 18 | 48130633 | C | T |
| 198 | DCC | rs1443570 | 18 | 48132617 | T | C |
| 199 | DCC | rs10502942 | 18 | 48132805 | C | T |
| 200 | DCC | rs1443568 | 18 | 48134239 | C | T |
| 201 | DCC | rs8088453 | 18 | 48138020 | C | T |
| 202 | DCC | rs3862675 | 18 | 48139134 | T | C |
| 203 | DCC | rs1443579 | 18 | 48141997 | G | A |
| 204 | DCC | rs1443578 | 18 | 48142237 | C | T |
| 205 | DCC | rs1431790 | 18 | 48146322 | C | T |
| 206 | DCC | rs11875845 | 18 | 48150106 | C | T |
| 207 | DCC | rs10502943 | 18 | 48154998 | A | C |
| 208 | DCC | rs11660123 | 18 | 48159350 | A | G |
| 209 | DCC | rs9967342 | 18 | 48161806 | T | C |
| 210 | DCC | rs7231326 | 18 | 48169407 | G | A |
| 211 | DCC | rs3862678 | 18 | 48176144 | G | A |
| 212 | DCC | rs12456286 | 18 | 48180441 | T | C |
| 213 | DCC | rs3862681 | 18 | 48184687 | T | C |
| 214 | DCC | rs3915401 | 18 | 48188603 | G | T |
| 215 | DCC | rs3862683 | 18 | 48189302 | T | C |
| 216 | DCC | rs3862684 | 18 | 48191248 | G | A |
| 217 | DCC | rs1560531 | 18 | 48191697 | T | C |
| 218 | DCC | rs1222938 | 18 | 48195163 | A | G |
| 219 | DCC | rs2960617 | 18 | 48202732 | A | C |
| 220 | DCC | rs17748116 | 18 | 48203971 | C | A |
| 221 | DCC | rs1145242 | 18 | 48209456 | T | C |
| 222 | DCC | rs10153352 | 18 | 48212689 | C | T |
| 223 | DCC | rs1367865 | 18 | 48215052 | G | A |
| 224 | DCC | rs1619323 | 18 | 48215947 | T | C |
| 225 | DCC | rs9304422 | 18 | 48216881 | C | T |
| 226 | DCC | rs347546 | 18 | 48231041 | C | T |
| 227 | DCC | rs347535 | 18 | 48238733 | T | C |
| 228 | DCC | rs12607228 | 18 | 48242130 | T | C |
| 229 | DCC | rs1221983 | 18 | 48247263 | C | A |
| 230 | DCC | rs1145245 | 18 | 48271108 | A | C |
| 231 | DCC | rs1221986 | 18 | 48272439 | G | A |
| 232 | DCC | rs7238242 | 18 | 48276298 | G | A |
| 233 | DCC | rs2168159 | 18 | 48280912 | G | A |
| 234 | DCC | rs7243931 | 18 | 48284719 | A | G |
| 235 | DCC | rs1031062 | 18 | 48292119 | A | C |
| 236 | DCC | rs2045154 | 18 | 48293673 | A | C |
| 237 | DCC | rs1302850 | 18 | 48297402 | T | G |
| 238 | DCC | rs1301943 | 18 | 48297690 | A | G |
| 239 | DCC | rs1144055 | 18 | 48300269 | A | C |
| 240 | DCC | rs1144063 | 18 | 48302679 | A | C |
| 241 | DCC | rs1454731 | 18 | 48334364 | A | G |
| 242 | DCC | rs1144081 | 18 | 48341900 | T | C |
| 243 | DCC | rs7231796 | 18 | 48342016 | C | A |
| 244 | DCC | rs993927 | 18 | 48355291 | G | T |
| 245 | DCC | rs9959870 | 18 | 48356800 | G | A |
| 246 | DCC | rs1143768 | 18 | 48361974 | C | T |
| 247 | DCC | rs4940191 | 18 | 48362227 | G | A |
| 248 | DCC | rs948590 | 18 | 48378925 | G | A |
| 249 | DCC | rs1221872 | 18 | 48384334 | G | A |
| 250 | DCC | rs1221874 | 18 | 48385107 | G | A |
| 251 | DCC | rs1221875 | 18 | 48385266 | G | A |
| 252 | DCC | rs1221877 | 18 | 48387823 | G | A |
| 253 | DCC | rs1221883 | 18 | 48389262 | T | C |
| 254 | DCC | rs1221884 | 18 | 48389343 | G | A |
| 255 | DCC | rs1221885 | 18 | 48389400 | A | G |
| 256 | DCC | rs2219556 | 18 | 48399087 | T | C |
| 257 | DCC | rs1221868 | 18 | 48406226 | G | A |
| 258 | DCC | rs1221869 | 18 | 48406959 | C | T |
| 259 | DCC | rs1237775 | 18 | 48407571 | C | T |
| 260 | DCC | rs4940196 | 18 | 48408403 | G | A |
| 261 | DCC | rs1675225 | 18 | 48412075 | C | T |
| 262 | DCC | rs7231528 | 18 | 48412117 | T | C |
| 263 | DCC | rs11082932 | 18 | 48434136 | A | C |
| 264 | DCC | rs4940199 | 18 | 48434701 | T | C |
| 265 | DCC | rs1460191 | 18 | 48435189 | G | T |
| 266 | DCC | rs1466373 | 18 | 48439207 | C | T |
| 267 | DCC | rs17749090 | 18 | 48439454 | T | C |
| 268 | DCC | rs999170 | 18 | 48439466 | G | A |
| 269 | DCC | rs768204 | 18 | 48441870 | C | A |
| 270 | DCC | rs4556886 | 18 | 48441979 | A | G |
| 271 | DCC | rs7245154 | 18 | 48450414 | A | G |
| 272 | DCC | rs4542757 | 18 | 48452721 | T | C |
| 273 | DCC | rs1870580 | 18 | 48458401 | T | G |
| 274 | DCC | rs1460200 | 18 | 48462959 | T | C |
| 275 | DCC | rs4940206 | 18 | 48463793 | A | G |
| 276 | DCC | rs1460199 | 18 | 48465384 | T | G |
| 277 | DCC | rs1903276 | 18 | 48466257 | T | C |
| 278 | DCC | rs1460198 | 18 | 48466646 | G | T |
| 279 | DCC | rs7236766 | 18 | 48476793 | G | T |
| 280 | DCC | rs7226526 | 18 | 48478195 | C | T |
| 281 | DCC | rs16955409 | 18 | 48484954 | G | A |
| 282 | DCC | rs1160400 | 18 | 48485282 | A | G |
| 283 | DCC | rs898438 | 18 | 48485784 | A | G |
| 284 | DCC | rs2054289 | 18 | 48492951 | G | A |
| 285 | DCC | rs4641847 | 18 | 48503879 | A | C |
| 286 | DCC | rs1574039 | 18 | 48507737 | T | C |
| 287 | DCC | rs4939702 | 18 | 48512934 | A | G |
| 288 | DCC | rs6508153 | 18 | 48516960 | C | A |
| 289 | DCC | rs9304427 | 18 | 48519936 | C | T |
| 290 | DCC | rs6508158 | 18 | 48530377 | T | C |
| 291 | DCC | rs10083941 | 18 | 48536119 | C | T |
| 292 | DCC | rs4334396 | 18 | 48537082 | T | C |
| 293 | DCC | rs4530241 | 18 | 48537574 | A | G |
| 294 | DCC | rs768048 | 18 | 48539395 | C | T |
| 295 | DCC | rs8092051 | 18 | 48539909 | G | A |
| 296 | DCC | rs6508159 | 18 | 48542017 | C | A |
| 297 | DCC | rs10515956 | 18 | 48562429 | G | A |
| 298 | DCC | rs10469016 | 18 | 48580882 | G | A |
| 299 | DCC | rs4939705 | 18 | 48588361 | G | A |
| 300 | DCC | rs2096994 | 18 | 48601486 | G | T |
| 301 | DCC | rs12605728 | 18 | 48602573 | G | A |
| 302 | DCC | rs9957838 | 18 | 48605651 | T | C |
| 303 | DCC | rs11082952 | 18 | 48611792 | G | A |
| 304 | DCC | rs2116378 | 18 | 48615581 | G | A |
| 305 | DCC | rs1573411 | 18 | 48621409 | G | A |
| 306 | DCC | rs1030594 | 18 | 48628027 | A | G |
| 307 | DCC | rs7506794 | 18 | 48629589 | C | A |
| 308 | DCC | rs752870 | 18 | 48640332 | G | A |
| 309 | DCC | rs11665486 | 18 | 48654443 | C | T |
| 310 | DCC | rs1431748 | 18 | 48654505 | T | G |
| 311 | DCC | rs9304435 | 18 | 48670226 | T | C |
| 312 | DCC | rs1943108 | 18 | 48679019 | A | G |
| 313 | DCC | rs1943107 | 18 | 48679129 | T | C |
| 314 | DCC | rs1943093 | 18 | 48681221 | T | C |
| 315 | DCC | rs2156283 | 18 | 48684272 | A | G |
| 316 | DCC | rs3737396 | 18 | 48688991 | G | A |
| 317 | DCC | rs1541287 | 18 | 48690658 | C | T |
| 318 | DCC | rs9964756 | 18 | 48695814 | G | T |
| 319 | DCC | rs1943097 | 18 | 48696063 | G | T |
| 320 | DCC | rs2298654 | 18 | 48705818 | A | C |
| 321 | DCC | rs4940230 | 18 | 48750681 | A | C |
| 322 | DCC | rs9965121 | 18 | 48759637 | A | G |
| 323 | DCC | rs8088528 | 18 | 48768381 | G | A |
| 324 | DCC | rs7504990 | 18 | 48771773 | C | T |
| 325 | DCC | rs7242428 | 18 | 48782879 | A | G |
| 326 | DCC | rs7506454 | 18 | 48787668 | A | G |
| 327 | DCC | rs7505465 | 18 | 48798868 | C | T |
| 328 | DCC | rs4078288 | 18 | 48801248 | T | C |
| 329 | DCC | rs7504750 | 18 | 48801637 | A | C |
| 330 | DCC | rs9950125 | 18 | 48802541 | G | A |
| 331 | DCC | rs4940235 | 18 | 48807970 | A | C |
| 332 | DCC | rs6508191 | 18 | 48831703 | T | C |
| 333 | DCC | rs4132357 | 18 | 48834972 | A | G |
| 334 | DCC | rs11874659 | 18 | 48842257 | C | T |
| 335 | DCC | rs8084454 | 18 | 48842942 | T | A |
| 336 | DCC | rs4129439 | 18 | 48843080 | A | G |
| 337 | DCC | rs9964201 | 18 | 48854549 | C | A |
| 338 | DCC | rs10502963 | 18 | 48875452 | T | C |
| 339 | DCC | rs4321259 | 18 | 48877637 | G | A |
| 340 | DCC | rs4414555 | 18 | 48897809 | C | T |
| 341 | DCC | rs1893368 | 18 | 48916170 | A | G |
| 342 | DCC | rs8088857 | 18 | 48923781 | C | T |
| 343 | DCC | rs8092993 | 18 | 48924108 | G | A |
| 344 | DCC | rs4939723 | 18 | 48927086 | G | A |
| 345 | DCC | rs9955274 | 18 | 48934174 | G | A |
| 346 | DCC | rs4940244 | 18 | 48945835 | A | G |
| 347 | DCC | rs6417121 | 18 | 48957743 | A | G |
| 348 | DCC | rs10775504 | 18 | 48960244 | G | A |
| 349 | DCC | rs10502962 | 18 | 48965411 | A | G |
| 350 | DCC | rs7238728 | 18 | 48966744 | G | A |
| 351 | DCC | rs3794923 | 18 | 48981515 | G | A |
| 352 | DCC | rs11662175 | 18 | 48982984 | T | C |
| 353 | DCC | rs9967014 | 18 | 48983808 | A | G |
| 354 | DCC | rs11082972 | 18 | 48987147 | T | C |
| 355 | DCC | rs12958954 | 18 | 48995506 | A | T |
| 356 | DCC | rs12964204 | 18 | 48996381 | G | A |
| 357 | DCC | rs9951763 | 18 | 48996517 | G | A |
| 358 | DCC | rs7243444 | 18 | 48997411 | G | A |
| 359 | DCC | rs4609919 | 18 | 48998836 | G | A |
| 360 | DCC | rs10502966 | 18 | 49002496 | A | G |
| 361 | DCC | rs7243756 | 18 | 49004872 | G | A |
| 362 | DCC | rs1431187 | 18 | 49057973 | G | A |
| 363 | DCC | rs1988344 | 18 | 49076735 | A | G |
| 364 | DCC | rs6508218 | 18 | 49078002 | A | G |
| 365 | DCC | rs6508220 | 18 | 49085173 | A | G |
| 366 | DCC | rs4940251 | 18 | 49087079 | A | C |
| 367 | DCC | rs8098084 | 18 | 49100966 | G | C |
| 368 | DCC | rs11872471 | 18 | 49114529 | G | T |
| 369 | DCC | rs9950970 | 18 | 49117805 | T | C |
| 370 | DCC | rs1367634 | 18 | 49118665 | C | A |
| 371 | DCC | rs12954274 | 18 | 49119871 | T | C |
| 372 | DCC | rs12968567 | 18 | 49127465 | C | T |
| 373 | DCC | rs11874922 | 18 | 49140165 | A | G |
| 374 | DCC | rs10502969 | 18 | 49141124 | A | C |
| 375 | DCC | rs10502970 | 18 | 49141838 | T | C |
| 376 | DCC | rs8095929 | 18 | 49148197 | T | C |
| 377 | DCC | rs8091068 | 18 | 49154429 | C | T |
| 378 | DCC | rs1910713 | 18 | 49158636 | C | A |
| 379 | DCC | rs1394466 | 18 | 49159203 | G | A |
| 380 | DCC | rs3764496 | 18 | 49166349 | G | A |
| 381 | DCC | rs3794922 | 18 | 49168244 | T | C |
| 382 | DCC | rs3794917 | 18 | 49170339 | A | C |
| 383 | DCC | rs1553334 | 18 | 49182224 | G | A |
| 384 | DCC | rs1010360 | 18 | 49182438 | A | C |
| 385 | DCC | rs11082993 | 18 | 49189350 | A | G |
| 386 | DCC | rs10853631 | 18 | 49190777 | A | G |
| 387 | DCC | rs10502975 | 18 | 49192238 | G | C |
| 388 | DCC | rs11876154 | 18 | 49194255 | A | G |
| 389 | DCC | rs10502976 | 18 | 49195959 | A | C |
| 390 | DCC | rs2270951 | 18 | 49196650 | A | G |
| 391 | DCC | rs9807370 | 18 | 49197242 | A | G |
| 392 | DCC | rs8095329 | 18 | 49213353 | A | G |
| 393 | DCC | rs12953529 | 18 | 49222970 | A | G |
| 394 | DCC | rs12604940 | 18 | 49226212 | A | G |
| 395 | DCC | rs869224 | 18 | 49231795 | C | T |
| 396 | DCC | rs1948783 | 18 | 49252758 | T | A |
| 397 | DCC | rs8082807 | 18 | 49264899 | A | C |
| 398 | DCC | rs1502223 | 18 | 49278513 | T | C |
| 399 | DCC | rs7232617 | 18 | 49278803 | C | T |
| 400 | DCC | rs1105642 | 18 | 49281084 | G | A |
| 401 | DCC | rs870508 | 18 | 49281552 | T | G |
| 402 | DCC | rs4453579 | 18 | 49301830 | G | A |
| 403 | DCC | rs1502219 | 18 | 49308886 | G | A |
| 404 | DCC | rs4245260 | 18 | 49309884 | T | C |
| 405 | DCC | rs2270954 | 18 | 49311295 | C | A |
| 406 | DPYSL2 | rs6998628 | 8 | 26489479 | G | A |
| 407 | DPYSL2 | rs6983262 | 8 | 26489802 | C | T |
| 408 | DPYSL2 | rs920697 | 8 | 26492992 | A | G |
| 409 | DPYSL2 | rs388047 | 8 | 26496145 | C | T |
| 410 | DPYSL2 | rs408753 | 8 | 26497478 | T | C |
| 411 | DPYSL2 | rs385351 | 8 | 26498999 | A | T |
| 412 | DPYSL2 | rs327234 | 8 | 26500585 | A | G |
| 413 | DPYSL2 | rs7825468 | 8 | 26506389 | A | G |
| 414 | DPYSL2 | rs5029306 | 8 | 26509021 | C | T |
| 415 | DPYSL2 | rs327232 | 8 | 26511631 | C | T |
| 416 | DPYSL2 | rs11780026 | 8 | 26518540 | A | G |
| 417 | DPYSL2 | rs11775967 | 8 | 26522776 | C | A |
| 418 | DPYSL2 | rs2585459 | 8 | 26529540 | A | G |
| 419 | DPYSL2 | rs403185 | 8 | 26532637 | G | A |
| 420 | DPYSL2 | rs7829347 | 8 | 26536533 | T | C |
| 421 | DPYSL2 | rs1561818 | 8 | 26538745 | C | A |
| 422 | DPYSL2 | rs327225 | 8 | 26539106 | T | G |
| 423 | DPYSL2 | rs327227 | 8 | 26539394 | A | G |
| 424 | DPYSL2 | rs999772 | 8 | 26542236 | C | T |
| 425 | DPYSL2 | rs327216 | 8 | 26547379 | C | T |
| 426 | DPYSL2 | rs1867042 | 8 | 26548476 | T | G |
| 427 | DPYSL2 | rs930470 | 8 | 26548804 | A | G |
| 428 | DPYSL2 | rs1972921 | 8 | 26551656 | C | T |
| 429 | DPYSL2 | rs4733044 | 8 | 26552587 | C | G |
| 430 | DPYSL2 | rs13277175 | 8 | 26553929 | T | G |
| 431 | DPYSL2 | rs4733048 | 8 | 26555005 | T | C |
| 432 | DPYSL2 | rs10093591 | 8 | 26556546 | A | G |
| 433 | DPYSL2 | rs9644117 | 8 | 26561434 | C | T |
| 434 | DPYSL2 | rs6992095 | 8 | 26564683 | T | C |
| 435 | DPYSL2 | rs11863 | 8 | 26570232 | C | T |
| 436 | DPYSL2 | rs920633 | 8 | 26570695 | C | T |
| 437 | DPYSL2 | rs17666 | 8 | 26571374 | T | C |
| 438 | DPYSL5 | rs6547284 | 2 | 26923865 | C | A |
| 439 | DPYSL5 | rs12615509 | 2 | 26952269 | G | A |
| 440 | DPYSL5 | rs7596707 | 2 | 26954784 | C | T |
| 441 | DPYSL5 | rs1371614 | 2 | 27006377 | C | T |
| 442 | DPYSL5 | rs3739088 | 2 | 27021287 | C | T |
| 443 | EFNA1 | rs4971066 | 1 | 153372505 | T | G |
| 444 | EFNA1 | rs9297 | 1 | 153373173 | A | G |
| 445 | EFNA2 | rs10424501 | 19 | 1248501 | G | A |
| 446 | EFNA3 | rs3766916 | 1 | 153316025 | C | T |
| 447 | EFNA3 | rs1462855 | 1 | 153320342 | T | C |
| 448 | EFNA3 | rs1870939 | 1 | 153322119 | G | A |
| 449 | EFNA3 | rs2306124 | 1 | 153326474 | A | G |
| 450 | EFNA4 | rs4045192 | 1 | 153305379 | A | G |
| 451 | EFNA5 | rs252815 | 5 | 106749581 | T | C |
| 452 | EFNA5 | rs252816 | 5 | 106752309 | A | G |
| 453 | EFNA5 | rs252818 | 5 | 106753589 | T | C |
| 454 | EFNA5 | rs461914 | 5 | 106771541 | T | C |
| 455 | EFNA5 | rs3797521 | 5 | 106775826 | G | A |
| 456 | EFNA5 | rs6868410 | 5 | 106777588 | A | G |
| 457 | EFNA5 | rs185221 | 5 | 106781603 | G | A |
| 458 | EFNA5 | rs252801 | 5 | 106785297 | A | G |
| 459 | EFNA5 | rs252802 | 5 | 106790482 | C | T |
| 460 | EFNA5 | rs252991 | 5 | 106795244 | G | A |
| 461 | EFNA5 | rs154648 | 5 | 106795835 | C | T |
| 462 | EFNA5 | rs152608 | 5 | 106799107 | G | A |
| 463 | EFNA5 | rs2283 | 5 | 106801521 | T | C |
| 464 | EFNA5 | rs152600 | 5 | 106805392 | T | G |
| 465 | EFNA5 | rs152599 | 5 | 106807203 | G | T |
| 466 | EFNA5 | rs537500 | 5 | 106821489 | C | T |
| 467 | EFNA5 | rs3909464 | 5 | 106821671 | A | C |
| 468 | EFNA5 | rs152581 | 5 | 106822182 | C | G |
| 469 | EFNA5 | rs152580 | 5 | 106823760 | A | G |
| 470 | EFNA5 | rs10491390 | 5 | 106828046 | G | A |
| 471 | EFNA5 | rs26247 | 5 | 106832261 | T | C |
| 472 | EFNA5 | rs2288385 | 5 | 106833949 | A | C |
| 473 | EFNA5 | rs636137 | 5 | 106834122 | C | T |
| 474 | EFNA5 | rs25994 | 5 | 106839543 | G | A |
| 475 | EFNA5 | rs26242 | 5 | 106842540 | C | T |
| 476 | EFNA5 | rs26244 | 5 | 106846241 | G | A |
| 477 | EFNA5 | rs25970 | 5 | 106854311 | C | T |
| 478 | EFNA5 | rs31717 | 5 | 106857437 | C | A |
| 479 | EFNA5 | rs27681 | 5 | 106859472 | G | A |
| 480 | EFNA5 | rs1196516 | 5 | 106862465 | G | A |
| 481 | EFNA5 | rs10491393 | 5 | 106864672 | A | G |
| 482 | EFNA5 | rs26249 | 5 | 106866305 | A | G |
| 483 | EFNA5 | rs10491394 | 5 | 106872849 | T | C |
| 484 | EFNA5 | rs152558 | 5 | 106875560 | A | G |
| 485 | EFNA5 | rs152555 | 5 | 106877572 | T | C |
| 486 | EFNA5 | rs250253 | 5 | 106880864 | C | T |
| 487 | EFNA5 | rs848827 | 5 | 106887382 | G | A |
| 488 | EFNA5 | rs464250 | 5 | 106891613 | A | C |
| 489 | EFNA5 | rs485241 | 5 | 106897222 | C | T |
| 490 | EFNA5 | rs662627 | 5 | 106909231 | C | T |
| 491 | EFNA5 | rs849520 | 5 | 106909445 | C | T |
| 492 | EFNA5 | rs553363 | 5 | 106909904 | C | T |
| 493 | EFNA5 | rs467089 | 5 | 106913812 | A | G |
| 494 | EFNA5 | rs341272 | 5 | 106914966 | A | G |
| 495 | EFNA5 | rs163888 | 5 | 106917834 | G | A |
| 496 | EFNA5 | rs352603 | 5 | 106921901 | G | A |
| 497 | EFNA5 | rs677797 | 5 | 106924382 | G | A |
| 498 | EFNA5 | rs164699 | 5 | 106925875 | T | C |
| 499 | EFNA5 | rs164700 | 5 | 106936631 | G | T |
| 500 | EFNA5 | rs6881619 | 5 | 106936958 | T | C |
| 501 | EFNA5 | rs6888194 | 5 | 106938555 | T | C |
| 502 | EFNA5 | rs12519552 | 5 | 106938808 | A | G |
| 503 | EFNA5 | rs607976 | 5 | 106941382 | C | T |
| 504 | EFNA5 | rs597083 | 5 | 106941487 | G | A |
| 505 | EFNA5 | rs641406 | 5 | 106941925 | A | G |
| 506 | EFNA5 | rs180791 | 5 | 106951467 | G | A |
| 507 | EFNA5 | rs9328010 | 5 | 106952727 | G | A |
| 508 | EFNA5 | rs3864269 | 5 | 106964863 | G | A |
| 509 | EFNA5 | rs17160255 | 5 | 106979255 | G | A |
| 510 | EFNA5 | rs4090594 | 5 | 106984341 | T | C |
| 511 | EFNA5 | rs164694 | 5 | 106988722 | G | A |
| 512 | EFNA5 | rs164840 | 5 | 106990247 | A | C |
| 513 | EFNA5 | rs352602 | 5 | 106994230 | T | C |
| 514 | EFNA5 | rs27550 | 5 | 106999466 | C | A |
| 515 | EFNA5 | rs17160324 | 5 | 107012706 | G | A |
| 516 | EFNA5 | rs7732162 | 5 | 107018929 | C | T |
| 517 | EFNB1 | rs638408 | X | 67967739 | T | C |
| 518 | EFNB1 | rs877818 | X | 67970231 | G | A |
| 519 | EFNB1 | rs7890214 | X | 67976912 | C | — |
| 520 | EFNB2 | rs9520087 | 13 | 105942320 | G | A |
| 521 | EFNB2 | rs9520088 | 13 | 105947684 | T | C |
| 522 | EFNB2 | rs2391331 | 13 | 105955709 | T | C |
| 523 | EFNB2 | rs7983579 | 13 | 105957446 | T | G |
| 524 | EFNB2 | rs9520090 | 13 | 105963255 | G | C |
| 525 | EFNB2 | rs2391333 | 13 | 105964694 | C | T |
| 526 | EFNB2 | rs2391335 | 13 | 105969985 | G | T |
| 527 | EFNB2 | rs2893262 | 13 | 105970806 | G | A |
| 528 | EFNB2 | rs2391340 | 13 | 105971753 | G | T |
| 529 | EFNB2 | rs8000078 | 13 | 105977974 | G | T |
| 530 | EFNB2 | rs9301142 | 13 | 105980436 | T | C |
| 531 | EFNB2 | rs3818377 | 13 | 105984934 | G | A |
| 532 | EFNB3 | rs3744262 | 17 | 7554489 | T | C |
| 533 | EFNB3 | rs7141 | 17 | 7555325 | A | G |
| 534 | EPHA2 | rs1566146 | 1 | 16325802 | A | T |
| 535 | EPHA2 | rs10907223 | 1 | 16332331 | G | A |
| 536 | EPHA2 | rs11260745 | 1 | 16338589 | T | C |
| 537 | EPHA2 | rs3820609 | 1 | 16340231 | C | A |
| 538 | EPHA2 | rs3768293 | 1 | 16340510 | A | C |
| 539 | EPHA2 | rs4661709 | 1 | 16351767 | C | T |
| 540 | EPHA3 | rs7428438 | 3 | 89242446 | A | G |
| 541 | EPHA3 | rs12714732 | 3 | 89261773 | A | G |
| 542 | EPHA3 | rs7429534 | 3 | 89273998 | C | T |
| 543 | EPHA3 | rs9815025 | 3 | 89297902 | G | A |
| 544 | EPHA3 | rs7433757 | 3 | 89300018 | G | A |
| 545 | EPHA3 | rs4128782 | 3 | 89320421 | T | C |
| 546 | EPHA3 | rs12486049 | 3 | 89323969 | T | G |
| 547 | EPHA3 | rs4132275 | 3 | 89330570 | A | G |
| 548 | EPHA3 | rs9310113 | 3 | 89343386 | T | A |
| 549 | EPHA3 | rs7373232 | 3 | 89350955 | A | G |
| 550 | EPHA3 | rs6551407 | 3 | 89353294 | T | C |
| 551 | EPHA3 | rs6551408 | 3 | 89353427 | T | C |
| 552 | EPHA3 | rs6419884 | 3 | 89362156 | T | C |
| 553 | EPHA3 | rs1512912 | 3 | 89390620 | G | A |
| 554 | EPHA3 | rs11916939 | 3 | 89392095 | G | A |
| 555 | EPHA3 | rs993186 | 3 | 89397551 | A | C |
| 556 | EPHA3 | rs3946805 | 3 | 89399065 | T | C |
| 557 | EPHA3 | rs1040017 | 3 | 89412321 | A | C |
| 558 | EPHA3 | rs1512910 | 3 | 89415142 | T | C |
| 559 | EPHA3 | rs1512909 | 3 | 89419106 | G | A |
| 560 | EPHA3 | rs9832305 | 3 | 89475467 | T | C |
| 561 | EPHA3 | rs9842599 | 3 | 89489945 | A | G |
| 562 | EPHA3 | rs870898 | 3 | 89499753 | C | T |
| 563 | EPHA3 | rs6551410 | 3 | 89524994 | T | A |
| 564 | EPHA3 | rs6803870 | 3 | 89526473 | C | T |
| 565 | EPHA3 | rs3792572 | 3 | 89539244 | A | T |
| 566 | EPHA3 | rs7632502 | 3 | 89552142 | T | A |
| 567 | EPHA3 | rs1398197 | 3 | 89581117 | C | T |
| 568 | EPHA3 | rs1028011 | 3 | 89581308 | C | T |
| 569 | EPHA3 | rs1157607 | 3 | 89589053 | C | T |
| 570 | EPHA3 | rs1054750 | 3 | 89604414 | T | C |
| 571 | EPHA3 | rs12636275 | 3 | 89605727 | C | A |
| 572 | EPHA3 | rs12631313 | 3 | 89611150 | A | T |
| 573 | EPHA4 | rs9758 | 2 | 221991243 | T | A |
| 574 | EPHA4 | rs3087584 | 2 | 221991524 | T | C |
| 575 | EPHA4 | rs957466 | 2 | 221991747 | G | A |
| 576 | EPHA4 | rs957465 | 2 | 221991757 | G | A |
| 577 | EPHA4 | rs3770208 | 2 | 221992889 | G | A |
| 578 | EPHA4 | rs10498110 | 2 | 221996112 | T | C |
| 579 | EPHA4 | rs16862636 | 2 | 221996623 | T | C |
| 580 | EPHA4 | rs2303901 | 2 | 221999145 | T | C |
| 581 | EPHA4 | rs3770205 | 2 | 222004003 | G | T |
| 582 | EPHA4 | rs4340507 | 2 | 222005350 | C | T |
| 583 | EPHA4 | rs3770199 | 2 | 222008242 | G | A |
| 584 | EPHA4 | rs3213844 | 2 | 222009600 | G | A |
| 585 | EPHA4 | rs2303900 | 2 | 222009956 | G | A |
| 586 | EPHA4 | rs3770196 | 2 | 222013204 | A | G |
| 587 | EPHA4 | rs3815971 | 2 | 222015538 | A | T |
| 588 | EPHA4 | rs2288628 | 2 | 222015777 | C | A |
| 589 | EPHA4 | rs2052943 | 2 | 222019309 | C | T |
| 590 | EPHA4 | rs3770186 | 2 | 222020582 | A | T |
| 591 | EPHA4 | rs962430 | 2 | 222020645 | G | A |
| 592 | EPHA4 | rs3770181 | 2 | 222022585 | C | T |
| 593 | EPHA4 | rs3770178 | 2 | 222022725 | C | T |
| 594 | EPHA4 | rs3770176 | 2 | 222025788 | G | A |
| 595 | EPHA4 | rs3815970 | 2 | 222028309 | C | T |
| 596 | EPHA4 | rs2288627 | 2 | 222028670 | T | C |
| 597 | EPHA4 | rs919502 | 2 | 222033574 | A | G |
| 598 | EPHA4 | rs1430217 | 2 | 222037268 | T | A |
| 599 | EPHA4 | rs16862708 | 2 | 222038365 | G | A |
| 600 | EPHA4 | rs975194 | 2 | 222045869 | T | C |
| 601 | EPHA4 | rs1430211 | 2 | 222047077 | A | G |
| 602 | EPHA4 | rs7566787 | 2 | 222051048 | T | A |
| 603 | EPHA4 | rs3770149 | 2 | 222051135 | A | G |
| 604 | EPHA4 | rs1367241 | 2 | 222061656 | G | A |
| 605 | EPHA4 | rs6727259 | 2 | 222066535 | G | C |
| 606 | EPHA4 | rs10498114 | 2 | 222067152 | A | G |
| 607 | EPHA4 | rs7598307 | 2 | 222077228 | T | C |
| 608 | EPHA4 | rs7575433 | 2 | 222078113 | G | A |
| 609 | EPHA4 | rs9288570 | 2 | 222087237 | G | A |
| 610 | EPHA4 | rs2710507 | 2 | 222089043 | G | A |
| 611 | EPHA4 | rs2250204 | 2 | 222108466 | T | C |
| 612 | EPHA4 | rs2710497 | 2 | 222114811 | C | T |
| 613 | EPHA4 | rs10498117 | 2 | 222120695 | C | T |
| 614 | EPHA4 | rs1568359 | 2 | 222120780 | G | A |
| 615 | EPHA4 | rs10498118 | 2 | 222131632 | G | A |
| 616 | EPHA4 | rs10498120 | 2 | 222133863 | A | G |
| 617 | EPHA4 | rs2056290 | 2 | 222136114 | C | T |
| 618 | EPHA4 | rs3770143 | 2 | 222139226 | A | G |
| 619 | EPHA5 | rs7688941 | 4 | 65879216 | C | A |
| 620 | EPHA5 | rs7349683 | 4 | 65880398 | C | T |
| 621 | EPHA5 | rs11945873 | 4 | 65881468 | A | G |
| 622 | EPHA5 | rs986208 | 4 | 65889086 | T | C |
| 623 | EPHA5 | rs2290254 | 4 | 65896637 | T | C |
| 624 | EPHA5 | rs1349446 | 4 | 65900928 | G | A |
| 625 | EPHA5 | rs1451179 | 4 | 65902236 | T | A |
| 626 | EPHA5 | rs1901264 | 4 | 65910336 | A | G |
| 627 | EPHA5 | rs13135753 | 4 | 65911321 | G | A |
| 628 | EPHA5 | rs12644356 | 4 | 65915355 | G | T |
| 629 | EPHA5 | rs7697278 | 4 | 65925720 | T | C |
| 630 | EPHA5 | rs1451173 | 4 | 65929032 | A | G |
| 631 | EPHA5 | rs1376416 | 4 | 65940867 | A | G |
| 632 | EPHA5 | rs6828375 | 4 | 65943120 | A | G |
| 633 | EPHA5 | rs1376412 | 4 | 65949609 | A | G |
| 634 | EPHA5 | rs7659284 | 4 | 65952367 | T | C |
| 635 | EPHA5 | rs2198103 | 4 | 65954773 | T | C |
| 636 | EPHA5 | rs13120093 | 4 | 65955005 | C | T |
| 637 | EPHA5 | rs2305351 | 4 | 65962765 | C | T |
| 638 | EPHA5 | rs1451171 | 4 | 65965920 | A | C |
| 639 | EPHA5 | rs6551922 | 4 | 65968694 | C | T |
| 640 | EPHA5 | rs12502991 | 4 | 65978142 | A | C |
| 641 | EPHA5 | rs6831652 | 4 | 65983019 | C | T |
| 642 | EPHA5 | rs10013782 | 4 | 65987556 | A | G |
| 643 | EPHA5 | rs10003638 | 4 | 65987744 | C | A |
| 644 | EPHA5 | rs10006892 | 4 | 65991075 | A | G |
| 645 | EPHA5 | rs9991256 | 4 | 66010448 | G | A |
| 646 | EPHA5 | rs4437303 | 4 | 66015513 | G | T |
| 647 | EPHA5 | rs4425430 | 4 | 66023561 | A | G |
| 648 | EPHA5 | rs4637457 | 4 | 66026264 | T | G |
| 649 | EPHA5 | rs11936348 | 4 | 66027548 | C | T |
| 650 | EPHA5 | rs10022719 | 4 | 66039479 | A | T |
| 651 | EPHA5 | rs4337775 | 4 | 66042171 | G | A |
| 652 | EPHA5 | rs6815080 | 4 | 66045081 | C | T |
| 653 | EPHA5 | rs7654891 | 4 | 66057227 | A | G |
| 654 | EPHA5 | rs4467633 | 4 | 66062314 | C | T |
| 655 | EPHA5 | rs12504386 | 4 | 66063457 | C | T |
| 656 | EPHA5 | rs4132313 | 4 | 66064175 | T | C |
| 657 | EPHA5 | rs11731307 | 4 | 66071595 | C | T |
| 658 | EPHA5 | rs7684022 | 4 | 66072295 | A | G |
| 659 | EPHA5 | rs6551932 | 4 | 66082433 | C | A |
| 660 | EPHA5 | rs6551933 | 4 | 66086161 | G | A |
| 661 | EPHA5 | rs13134593 | 4 | 66093687 | A | C |
| 662 | EPHA5 | rs4518304 | 4 | 66104260 | G | A |
| 663 | EPHA5 | rs11931601 | 4 | 66114503 | T | C |
| 664 | EPHA5 | rs9994092 | 4 | 66118708 | C | A |
| 665 | EPHA5 | rs13127631 | 4 | 66126468 | G | T |
| 666 | EPHA5 | rs4860188 | 4 | 66150975 | C | T |
| 667 | EPHA5 | rs11131604 | 4 | 66152034 | C | T |
| 668 | EPHA5 | rs4404602 | 4 | 66153407 | T | C |
| 669 | EPHA5 | rs6816785 | 4 | 66157875 | A | C |
| 670 | EPHA5 | rs7697951 | 4 | 66169735 | T | C |
| 671 | EPHA5 | rs4422467 | 4 | 66177239 | G | A |
| 672 | EPHA5 | rs4438803 | 4 | 66190037 | G | T |
| 673 | EPHA5 | rs4611973 | 4 | 66198990 | G | A |
| 674 | EPHA5 | rs4441811 | 4 | 66204083 | C | T |
| 675 | EPHA5 | rs7655988 | 4 | 66210549 | C | T |
| 676 | EPHA7 | rs431650 | 6 | 94010598 | C | T |
| 677 | EPHA7 | rs3823231 | 6 | 94016081 | A | G |
| 678 | EPHA7 | rs1386274 | 6 | 94018291 | T | C |
| 679 | EPHA7 | rs404201 | 6 | 94018612 | C | T |
| 680 | EPHA7 | rs17447037 | 6 | 94019482 | C | T |
| 681 | EPHA7 | rs345737 | 6 | 94020907 | A | G |
| 682 | EPHA7 | rs10806474 | 6 | 94034628 | T | C |
| 683 | EPHA7 | rs345713 | 6 | 94038844 | C | T |
| 684 | EPHA7 | rs1548297 | 6 | 94039774 | T | C |
| 685 | EPHA7 | rs345718 | 6 | 94043820 | T | C |
| 686 | EPHA7 | rs967520 | 6 | 94057233 | A | C |
| 687 | EPHA7 | rs346462 | 6 | 94061205 | A | G |
| 688 | EPHA7 | rs9363058 | 6 | 94065255 | C | T |
| 689 | EPHA7 | rs345716 | 6 | 94068961 | A | C |
| 690 | EPHA7 | rs164294 | 6 | 94082304 | G | A |
| 691 | EPHA7 | rs164298 | 6 | 94085685 | G | A |
| 692 | EPHA7 | rs164301 | 6 | 94086535 | G | T |
| 693 | EPHA7 | rs9294570 | 6 | 94093450 | G | A |
| 694 | EPHA7 | rs13200186 | 6 | 94106054 | C | T |
| 695 | EPHA7 | rs4706437 | 6 | 94107892 | G | A |
| 696 | EPHA7 | rs13203837 | 6 | 94108632 | A | C |
| 697 | EPHA7 | rs4707795 | 6 | 94111588 | G | A |
| 698 | EPHA7 | rs361505 | 6 | 94113922 | T | C |
| 699 | EPHA7 | rs164538 | 6 | 94117089 | T | C |
| 700 | EPHA7 | rs164540 | 6 | 94121126 | A | G |
| 701 | EPHA7 | rs164544 | 6 | 94124701 | A | G |
| 702 | EPHA7 | rs164546 | 6 | 94125428 | A | G |
| 703 | EPHA7 | rs164548 | 6 | 94128328 | G | A |
| 704 | EPHA7 | rs9354011 | 6 | 94132167 | G | A |
| 705 | EPHA7 | rs3778470 | 6 | 94132404 | C | T |
| 706 | EPHA7 | rs351328 | 6 | 94139843 | C | A |
| 707 | EPHA7 | rs549965 | 6 | 94161247 | C | T |
| 708 | EPHA7 | rs564158 | 6 | 94165593 | A | G |
| 709 | EPHA7 | rs3799792 | 6 | 94174266 | A | G |
| 710 | EPHA7 | rs1492968 | 6 | 94176629 | T | C |
| 711 | EPHA7 | rs474809 | 6 | 94176912 | G | A |
| 712 | EPHA7 | rs2278107 | 6 | 94177359 | T | C |
| 713 | EPHA7 | rs351329 | 6 | 94180877 | G | T |
| 714 | EPHA7 | rs575554 | 6 | 94182712 | G | A |
| 715 | EPHA7 | rs351331 | 6 | 94186744 | C | T |
| 716 | EPHA7 | rs351332 | 6 | 94186764 | T | C |
| 717 | EPHA8 | rs209736 | 1 | 22763995 | T | C |
| 718 | EPHA8 | rs209741 | 1 | 22771597 | A | G |
| 719 | EPHA8 | rs11580191 | 1 | 22775109 | C | T |
| 720 | EPHA8 | rs649754 | 1 | 22778833 | T | G |
| 721 | EPHA8 | rs606002 | 1 | 22788339 | A | G |
| 722 | EPHA8 | rs521570 | 1 | 22788564 | T | A |
| 723 | EPHA8 | rs671485 | 1 | 22789450 | A | G |
| 724 | EPHA8 | rs209696 | 1 | 22792490 | C | T |
| 725 | EPHA8 | rs209693 | 1 | 22794612 | T | G |
| 726 | EPHA8 | rs663002 | 1 | 22798199 | C | A |
| 727 | EPHA8 | rs209697 | 1 | 22801962 | T | C |
| 728 | EPHB1 | rs3732568 | 3 | 135998025 | C | A |
| 729 | EPHB1 | rs7648359 | 3 | 136006297 | C | T |
| 730 | EPHB1 | rs4955502 | 3 | 136015473 | T | C |
| 731 | EPHB1 | rs6802089 | 3 | 136026988 | C | T |
| 732 | EPHB1 | rs6789449 | 3 | 136027011 | A | G |
| 733 | EPHB1 | rs885594 | 3 | 136034217 | T | C |
| 734 | EPHB1 | rs2138213 | 3 | 136036852 | T | C |
| 735 | EPHB1 | rs3732569 | 3 | 136043481 | A | G |
| 736 | EPHB1 | rs12489360 | 3 | 136057408 | A | G |
| 737 | EPHB1 | rs868130 | 3 | 136061912 | C | T |
| 738 | EPHB1 | rs7651593 | 3 | 136067466 | A | C |
| 739 | EPHB1 | rs6762261 | 3 | 136073827 | C | T |
| 740 | EPHB1 | rs1515367 | 3 | 136075129 | G | T |
| 741 | EPHB1 | rs1399709 | 3 | 136086266 | C | T |
| 742 | EPHB1 | rs9868348 | 3 | 136089932 | C | T |
| 743 | EPHB1 | rs6786490 | 3 | 136096277 | C | A |
| 744 | EPHB1 | rs1996242 | 3 | 136101508 | T | C |
| 745 | EPHB1 | rs36221 | 3 | 136107329 | C | T |
| 746 | EPHB1 | rs10512935 | 3 | 136107944 | G | A |
| 747 | EPHB1 | rs36218 | 3 | 136109028 | C | T |
| 748 | EPHB1 | rs11709198 | 3 | 136119213 | A | C |
| 749 | EPHB1 | rs36198 | 3 | 136119485 | G | A |
| 750 | EPHB1 | rs11719058 | 3 | 136120635 | C | T |
| 751 | EPHB1 | rs11920780 | 3 | 136125356 | T | C |
| 752 | EPHB1 | rs635721 | 3 | 136127325 | T | G |
| 753 | EPHB1 | rs36178 | 3 | 136129858 | A | G |
| 754 | EPHB1 | rs40437 | 3 | 136133621 | C | T |
| 755 | EPHB1 | rs6788931 | 3 | 136139313 | G | A |
| 756 | EPHB1 | rs984709 | 3 | 136140541 | G | A |
| 757 | EPHB1 | rs9874260 | 3 | 136142240 | T | C |
| 758 | EPHB1 | rs983401 | 3 | 136144119 | A | C |
| 759 | EPHB1 | rs975149 | 3 | 136149164 | T | C |
| 760 | EPHB1 | rs972234 | 3 | 136151448 | C | T |
| 761 | EPHB1 | rs7644369 | 3 | 136153213 | C | T |
| 762 | EPHB1 | rs9815290 | 3 | 136154448 | G | A |
| 763 | EPHB1 | rs6781640 | 3 | 136164136 | T | C |
| 764 | EPHB1 | rs574719 | 3 | 136178135 | A | G |
| 765 | EPHB1 | rs39699 | 3 | 136180873 | C | T |
| 766 | EPHB1 | rs6776069 | 3 | 136181558 | T | C |
| 767 | EPHB1 | rs9855772 | 3 | 136187701 | T | C |
| 768 | EPHB1 | rs7613213 | 3 | 136192640 | C | T |
| 769 | EPHB1 | rs36074 | 3 | 136202104 | G | A |
| 770 | EPHB1 | rs36073 | 3 | 136202843 | G | A |
| 771 | EPHB1 | rs41182 | 3 | 136208970 | C | T |
| 772 | EPHB1 | rs36059 | 3 | 136215421 | G | T |
| 773 | EPHB1 | rs36057 | 3 | 136219360 | A | C |
| 774 | EPHB1 | rs36054 | 3 | 136223141 | C | T |
| 775 | EPHB1 | rs10935153 | 3 | 136235614 | C | A |
| 776 | EPHB1 | rs36039 | 3 | 136238280 | C | A |
| 777 | EPHB1 | rs6791224 | 3 | 136246149 | G | A |
| 778 | EPHB1 | rs6793611 | 3 | 136246206 | G | A |
| 779 | EPHB1 | rs36041 | 3 | 136248987 | C | T |
| 780 | EPHB1 | rs253876 | 3 | 136251604 | A | G |
| 781 | EPHB1 | rs12639432 | 3 | 136253209 | C | T |
| 782 | EPHB1 | rs6778165 | 3 | 136261181 | G | A |
| 783 | EPHB1 | rs6439552 | 3 | 136275533 | C | T |
| 784 | EPHB1 | rs6439553 | 3 | 136288270 | C | A |
| 785 | EPHB1 | rs959562 | 3 | 136288461 | T | C |
| 786 | EPHB1 | rs2030737 | 3 | 136292066 | A | G |
| 787 | EPHB1 | rs9847974 | 3 | 136299517 | A | C |
| 788 | EPHB1 | rs11711351 | 3 | 136303818 | G | A |
| 789 | EPHB1 | rs7372127 | 3 | 136304088 | A | C |
| 790 | EPHB1 | rs3732567 | 3 | 136308201 | C | G |
| 791 | EPHB1 | rs7374961 | 3 | 136308677 | A | G |
| 792 | EPHB1 | rs7374193 | 3 | 136312227 | G | A |
| 793 | EPHB1 | rs9813334 | 3 | 136312251 | A | G |
| 794 | EPHB1 | rs13074164 | 3 | 136319363 | A | G |
| 795 | EPHB1 | rs9864760 | 3 | 136319728 | G | A |
| 796 | EPHB1 | rs11706256 | 3 | 136324141 | C | T |
| 797 | EPHB1 | rs940639 | 3 | 136325112 | C | T |
| 798 | EPHB1 | rs7625747 | 3 | 136328382 | T | G |
| 799 | EPHB1 | rs1404579 | 3 | 136329499 | T | C |
| 800 | EPHB1 | rs1404577 | 3 | 136331640 | C | T |
| 801 | EPHB1 | rs940638 | 3 | 136333460 | A | G |
| 802 | EPHB1 | rs6772933 | 3 | 136335378 | A | G |
| 803 | EPHB1 | rs12488683 | 3 | 136337632 | G | A |
| 804 | EPHB1 | rs868909 | 3 | 136342523 | C | T |
| 805 | EPHB1 | rs3772647 | 3 | 136347218 | T | C |
| 806 | EPHB1 | rs3821504 | 3 | 136347567 | T | G |
| 807 | EPHB1 | rs12492126 | 3 | 136354264 | A | G |
| 808 | EPHB1 | rs2291989 | 3 | 136355229 | C | T |
| 809 | EPHB1 | rs7642038 | 3 | 136360324 | A | G |
| 810 | EPHB1 | rs7620133 | 3 | 136360370 | C | T |
| 811 | EPHB1 | rs1501205 | 3 | 136362243 | T | C |
| 812 | EPHB1 | rs905047 | 3 | 136364922 | A | C |
| 813 | EPHB1 | rs905048 | 3 | 136364978 | A | G |
| 814 | EPHB1 | rs3772638 | 3 | 136367162 | G | A |
| 815 | EPHB1 | rs4894248 | 3 | 136369579 | C | A |
| 816 | EPHB1 | rs6801620 | 3 | 136375175 | C | T |
| 817 | EPHB1 | rs10512944 | 3 | 136381033 | C | T |
| 818 | EPHB1 | rs10512945 | 3 | 136381649 | G | A |
| 819 | EPHB1 | rs9816567 | 3 | 136385156 | G | A |
| 820 | EPHB1 | rs6776570 | 3 | 136394606 | A | G |
| 821 | EPHB1 | rs3821502 | 3 | 136395256 | T | C |
| 822 | EPHB1 | rs6763009 | 3 | 136397877 | C | A |
| 823 | EPHB1 | rs6766459 | 3 | 136398732 | T | G |
| 824 | EPHB1 | rs6439563 | 3 | 136411406 | A | G |
| 825 | EPHB1 | rs931726 | 3 | 136413629 | C | T |
| 826 | EPHB1 | rs4309752 | 3 | 136416305 | G | A |
| 827 | EPHB1 | rs9858763 | 3 | 136419272 | A | G |
| 828 | EPHB1 | rs6802560 | 3 | 136419964 | T | G |
| 829 | EPHB1 | rs6777929 | 3 | 136420022 | G | A |
| 830 | EPHB1 | rs1392920 | 3 | 136424646 | G | A |
| 831 | EPHB1 | rs11917038 | 3 | 136427813 | C | T |
| 832 | EPHB1 | rs4894320 | 3 | 136428256 | G | A |
| 833 | EPHB1 | rs7613488 | 3 | 136428555 | A | G |
| 834 | EPHB1 | rs9871429 | 3 | 136433725 | C | A |
| 835 | EPHB1 | rs9865620 | 3 | 136438404 | G | A |
| 836 | EPHB1 | rs11719703 | 3 | 136440350 | A | G |
| 837 | EPHB1 | rs6793828 | 3 | 136443159 | T | C |
| 838 | EPHB1 | rs9874828 | 3 | 136443518 | T | A |
| 839 | EPHB1 | rs4569680 | 3 | 136445455 | T | C |
| 840 | EPHB1 | rs4456886 | 3 | 136452626 | C | T |
| 841 | EPHB1 | rs13085606 | 3 | 136459853 | T | C |
| 842 | EPHB1 | rs10608 | 3 | 136461858 | G | A |
| 843 | EPHB2 | rs734445 | 1 | 22917414 | T | C |
| 844 | EPHB2 | rs2869513 | 1 | 22918529 | T | C |
| 845 | EPHB2 | rs882025 | 1 | 22922938 | G | A |
| 846 | EPHB2 | rs4655093 | 1 | 22927946 | T | G |
| 847 | EPHB2 | rs1318720 | 1 | 22939098 | A | G |
| 848 | EPHB2 | rs876685 | 1 | 22945006 | A | G |
| 849 | EPHB2 | rs893964 | 1 | 22958682 | G | A |
| 850 | EPHB2 | rs7516175 | 1 | 22969404 | A | C |
| 851 | EPHB2 | rs12410046 | 1 | 22971568 | G | A |
| 852 | EPHB2 | rs1561624 | 1 | 22974274 | C | A |
| 853 | EPHB2 | rs2817902 | 1 | 22981450 | C | G |
| 854 | EPHB2 | rs2817901 | 1 | 22981877 | G | A |
| 855 | EPHB2 | rs750012 | 1 | 22983091 | C | T |
| 856 | EPHB2 | rs2817896 | 1 | 22988635 | A | G |
| 857 | EPHB2 | rs1982227 | 1 | 22994047 | C | G |
| 858 | EPHB2 | rs4506436 | 1 | 23000462 | C | T |
| 859 | EPHB2 | rs2582032 | 1 | 23004565 | G | A |
| 860 | EPHB2 | rs2869511 | 1 | 23013286 | A | G |
| 861 | EPHB2 | rs751022 | 1 | 23016233 | C | T |
| 862 | EPHB2 | rs893966 | 1 | 23016592 | G | A |
| 863 | EPHB2 | rs6667416 | 1 | 23017825 | G | A |
| 864 | EPHB2 | rs11800828 | 1 | 23044292 | A | G |
| 865 | EPHB2 | rs4655130 | 1 | 23048580 | A | G |
| 866 | EPHB2 | rs4654824 | 1 | 23055716 | C | T |
| 867 | EPHB2 | rs6670405 | 1 | 23061709 | T | C |
| 868 | EPHB2 | rs4655134 | 1 | 23071205 | T | G |
| 869 | EPHB2 | rs6426771 | 1 | 23077607 | G | A |
| 870 | EPHB2 | rs116119 | 1 | 23091773 | C | T |
| 871 | EPHB2 | rs2746331 | 1 | 23091930 | A | G |
| 872 | EPHB2 | rs2675494 | 1 | 23099752 | C | T |
| 873 | EPHB2 | rs309502 | 1 | 23104986 | A | G |
| 874 | EPHB2 | rs309499 | 1 | 23107532 | C | T |
| 875 | EPHB2 | rs309498 | 1 | 23108778 | C | A |
| 876 | EPHB2 | rs309495 | 1 | 23110609 | C | T |
| 877 | EPHB3 | rs11713341 | 3 | 185760796 | T | C |
| 878 | EPHB3 | rs4132006 | 3 | 185764349 | C | T |
| 879 | EPHB3 | rs9647371 | 3 | 185766972 | C | T |
| 880 | EPHB3 | rs9862375 | 3 | 185773041 | G | A |
| 881 | EPHB3 | rs9823034 | 3 | 185776303 | T | C |
| 882 | EPHB3 | rs7652597 | 3 | 185776462 | T | C |
| 883 | EPHB4 | rs314353 | 7 | 100244079 | C | T |
| 884 | EPHB4 | rs2230585 | 7 | 100248532 | C | T |
| 885 | EPHB4 | rs314358 | 7 | 100248592 | C | T |
| 886 | EPHB4 | rs144173 | 7 | 100254185 | C | T |
| 887 | EPHB4 | rs2250818 | 7 | 100260091 | G | A |
| 888 | EPHB4 | rs314310 | 7 | 100260104 | G | T |
| 889 | EPHB4 | rs314313 | 7 | 100261300 | A | G |
| 890 | EPHB6 | rs7789029 | 7 | 142262494 | A | C |
| 891 | EPHB6 | rs1009848 | 7 | 142265372 | T | C |
| 892 | EPHB6 | rs8177146 | 7 | 142273374 | T | G |
| 893 | EPHB6 | rs4987691 | 7 | 142278063 | T | C |
| 894 | FYN | rs6916861 | 6 | 112088949 | A | C |
| 895 | FYN | rs9387024 | 6 | 112090713 | G | C |
| 896 | FYN | rs2237252 | 6 | 112095917 | A | G |
| 897 | FYN | rs6919400 | 6 | 112098013 | T | C |
| 898 | FYN | rs9374273 | 6 | 112101515 | T | C |
| 899 | FYN | rs1998038 | 6 | 112113687 | C | T |
| 900 | FYN | rs11153311 | 6 | 112116017 | A | G |
| 901 | FYN | rs812500 | 6 | 112124140 | C | — |
| 902 | FYN | rs809193 | 6 | 112127106 | A | G |
| 903 | FYN | rs1022647 | 6 | 112136094 | C | T |
| 904 | FYN | rs1022648 | 6 | 112136296 | T | C |
| 905 | FYN | rs706882 | 6 | 112140062 | C | T |
| 906 | FYN | rs809192 | 6 | 112141694 | G | A |
| 907 | FYN | rs706895 | 6 | 112148051 | T | C |
| 908 | FYN | rs1740377 | 6 | 112149810 | C | A |
| 909 | FYN | rs1726113 | 6 | 112149825 | C | A |
| 910 | FYN | rs697642 | 6 | 112159797 | G | A |
| 911 | FYN | rs6568703 | 6 | 112172393 | T | A |
| 912 | FYN | rs6910116 | 6 | 112182620 | A | G |
| 913 | FYN | rs1465061 | 6 | 112188308 | G | A |
| 914 | FYN | rs11964650 | 6 | 112188630 | C | T |
| 915 | FYN | rs13218316 | 6 | 112189726 | C | T |
| 916 | FYN | rs11963612 | 6 | 112203037 | T | C |
| 917 | FYN | rs1405530 | 6 | 112204757 | C | T |
| 918 | FYN | rs9384805 | 6 | 112208417 | C | A |
| 919 | FYN | rs2148709 | 6 | 112227920 | G | A |
| 920 | FYN | rs2148710 | 6 | 112228918 | C | T |
| 921 | FYN | rs2182644 | 6 | 112245574 | G | A |
| 922 | FYN | rs9372313 | 6 | 112248782 | C | T |
| 923 | FYN | rs1327202 | 6 | 112259107 | G | A |
| 924 | FYN | rs7761071 | 6 | 112260713 | C | T |
| 925 | FYN | rs6923217 | 6 | 112263789 | T | C |
| 926 | FYN | rs7749147 | 6 | 112271005 | G | A |
| 927 | FYN | rs10499061 | 6 | 112288722 | C | T |
| 928 | FYN | rs9320379 | 6 | 112291313 | T | C |
| 929 | FYN | rs2024832 | 6 | 112296404 | T | G |
| 930 | FYN | rs1409837 | 6 | 112302303 | T | C |
| 931 | GNAI1 | rs2428469 | 7 | 79603557 | A | C |
| 932 | GNAI1 | rs2523189 | 7 | 79614363 | C | T |
| 933 | GNAI1 | rs10274707 | 7 | 79618227 | C | T |
| 934 | GNAI1 | rs2714454 | 7 | 79627115 | A | G |
| 935 | GNAI1 | rs2714449 | 7 | 79629882 | C | T |
| 936 | GNAI1 | rs1468242 | 7 | 79636559 | T | C |
| 937 | GNAI1 | rs916905 | 7 | 79638282 | T | C |
| 938 | GNAI1 | rs10230368 | 7 | 79647919 | A | G |
| 939 | GNAI1 | rs7808119 | 7 | 79654273 | G | T |
| 940 | GNAI1 | rs4512317 | 7 | 79655884 | A | C |
| 941 | GNAI1 | rs6467043 | 7 | 79660338 | T | C |
| 942 | GNAI1 | rs10272499 | 7 | 79667779 | C | T |
| 943 | GNAI1 | rs10248616 | 7 | 79679500 | T | G |
| 944 | GNAI1 | rs10238438 | 7 | 79679889 | A | G |
| 945 | GNAI1 | rs2886611 | 7 | 79681240 | C | T |
| 946 | GNAI1 | rs11767465 | 7 | 79686229 | A | G |
| 947 | GNAI2 | rs2282751 | 3 | 50266788 | G | A |
| 948 | GNAI2 | rs9830408 | 3 | 50269554 | A | G |
| 949 | GNAI3 | rs3737182 | 1 | 109892793 | G | A |
| 950 | GNAI3 | rs518076 | 1 | 109910561 | A | G |
| 951 | GNAI3 | rs6537837 | 1 | 109921254 | C | T |
| 952 | GNAI3 | rs6682956 | 1 | 109931786 | G | A |
| 953 | GNAI3 | rs12033376 | 1 | 109932163 | A | T |
| 954 | GNAI3 | rs7537507 | 1 | 109932627 | G | A |
| 955 | GNAI3 | rs541320 | 1 | 109933620 | A | G |
| 956 | GNAI3 | rs2483920 | 1 | 109935294 | G | T |
| 957 | GNAI3 | rs2301230 | 1 | 109937980 | T | C |
| 958 | GNAI3 | rs3525 | 1 | 109938368 | T | A |
| 959 | GSK3B | rs2873950 | 3 | 121029751 | A | C |
| 960 | GSK3B | rs6805251 | 3 | 121043295 | C | T |
| 961 | GSK3B | rs1732170 | 3 | 121066365 | G | A |
| 962 | GSK3B | rs3108749 | 3 | 121077177 | C | T |
| 963 | GSK3B | rs1719895 | 3 | 121078192 | C | T |
| 964 | GSK3B | rs4340737 | 3 | 121104703 | C | T |
| 965 | GSK3B | rs9872321 | 3 | 121110997 | C | — |
| 966 | GSK3B | rs13314595 | 3 | 121113787 | C | T |
| 967 | GSK3B | rs6438552 | 3 | 121114503 | A | G |
| 968 | GSK3B | rs4072520 | 3 | 121118082 | C | A |
| 969 | GSK3B | rs6438553 | 3 | 121122718 | A | T |
| 970 | GSK3B | rs7620750 | 3 | 121125870 | G | A |
| 971 | GSK3B | rs4688046 | 3 | 121146559 | T | C |
| 972 | GSK3B | rs4688054 | 3 | 121216399 | T | C |
| 973 | GSK3B | rs6770314 | 3 | 121225410 | G | A |
| 974 | GSK3B | rs11919783 | 3 | 121229322 | G | A |
| 975 | GSK3B | rs12630592 | 3 | 121250935 | G | T |
| 976 | GSK3B | rs2199503 | 3 | 121261178 | G | A |
| 977 | GSK3B | rs334566 | 3 | 121270682 | A | G |
| 978 | GSK3B | rs334559 | 3 | 121292294 | G | A |
| 979 | ITGB1 | rs2153875 | 10 | 33230572 | A | C |
| 980 | ITGB1 | rs2488318 | 10 | 33235174 | A | G |
| 981 | ITGB1 | rs3824602 | 10 | 33235882 | G | A |
| 982 | ITGB1 | rs2488320 | 10 | 33238916 | C | T |
| 983 | ITGB1 | rs4587680 | 10 | 33240787 | C | T |
| 984 | ITGB1 | rs2488326 | 10 | 33247502 | C | T |
| 985 | ITGB1 | rs2230396 | 10 | 33249271 | A | C |
| 986 | ITGB1 | rs3780873 | 10 | 33253685 | C | T |
| 987 | ITGB1 | rs2298141 | 10 | 33254807 | A | G |
| 988 | ITGB1 | rs2488331 | 10 | 33263769 | T | C |
| 989 | ITGB1 | rs2504001 | 10 | 33264892 | G | A |
| 990 | ITGB1 | rs2488336 | 10 | 33276310 | C | T |
| 991 | ITGB1 | rs1187070 | 10 | 33281143 | C | A |
| 992 | ITGB1 | rs11009157 | 10 | 33282406 | G | A |
| 993 | KRAS | rs13096 | 12 | 25251107 | G | A |
| 994 | KRAS | rs7960917 | 12 | 25252912 | T | C |
| 995 | KRAS | rs9266 | 12 | 25253483 | C | T |
| 996 | KRAS | rs4246229 | 12 | 25258935 | A | G |
| 997 | KRAS | rs11047901 | 12 | 25267595 | G | A |
| 998 | KRAS | rs12579942 | 12 | 25274460 | T | C |
| 999 | KRAS | rs2970532 | 12 | 25287301 | C | T |
| 1000 | KRAS | rs7312175 | 12 | 25295870 | G | A |
| 1001 | LIMK1 | rs6460071 | 7 | 73135131 | G | A |
| 1002 | LIMK1 | rs2855726 | 7 | 73155049 | G | A |
| 1003 | LIMK1 | rs2269081 | 7 | 73157822 | A | C |
| 1004 | LIMK1 | rs150862 | 7 | 73162142 | A | G |
| 1005 | LIMK1 | rs2285025 | 7 | 73167911 | T | C |
| 1006 | LIMK2 | rs5749237 | 22 | 29948957 | A | G |
| 1007 | LIMK2 | rs730286 | 22 | 29949701 | T | A |
| 1008 | LIMK2 | rs5997917 | 22 | 29951791 | G | A |
| 1009 | LIMK2 | rs5997919 | 22 | 29953441 | G | A |
| 1010 | LIMK2 | rs5997921 | 22 | 29960895 | G | T |
| 1011 | LIMK2 | rs713947 | 22 | 29969508 | A | C |
| 1012 | LIMK2 | rs2267168 | 22 | 29973479 | T | G |
| 1013 | LIMK2 | rs7287402 | 22 | 29984822 | T | C |
| 1014 | LIMK2 | rs2073856 | 22 | 29993984 | G | A |
| 1015 | LIMK2 | rs2413045 | 22 | 29995861 | G | A |
| 1016 | LIMK2 | rs2040532 | 22 | 29999266 | C | T |
| 1017 | LIMK2 | rs5753535 | 22 | 30003449 | A | G |
| 1018 | LIMK2 | rs2073860 | 22 | 30004199 | C | T |
| 1019 | LIMK2 | rs4141404 | 22 | 30005184 | G | T |
| 1020 | LRRC4C | rs2953310 | 11 | 40094118 | T | C |
| 1021 | LRRC4C | rs7930525 | 11 | 40099240 | C | T |
| 1022 | LRRC4C | rs11035695 | 11 | 40099919 | G | A |
| 1023 | LRRC4C | rs12281232 | 11 | 40111639 | T | G |
| 1024 | LRRC4C | rs7936167 | 11 | 40112634 | T | C |
| 1025 | LRRC4C | rs7952540 | 11 | 40118547 | G | A |
| 1026 | LRRC4C | rs11035715 | 11 | 40129547 | G | T |
| 1027 | LRRC4C | rs11035719 | 11 | 40137147 | A | C |
| 1028 | LRRC4C | rs10768581 | 11 | 40137677 | C | T |
| 1029 | LRRC4C | rs11035722 | 11 | 40145806 | A | G |
| 1030 | LRRC4C | rs4429048 | 11 | 40149101 | G | A |
| 1031 | LRRC4C | rs4237677 | 11 | 40149429 | A | C |
| 1032 | LRRC4C | rs7926722 | 11 | 40152668 | T | C |
| 1033 | LRRC4C | rs4319506 | 11 | 40161716 | T | C |
| 1034 | LRRC4C | rs4075903 | 11 | 40164498 | G | A |
| 1035 | LRRC4C | rs7924805 | 11 | 40167827 | C | T |
| 1036 | LRRC4C | rs10837363 | 11 | 40174632 | G | A |
| 1037 | LRRC4C | rs4073087 | 11 | 40176400 | A | G |
| 1038 | LRRC4C | rs7106166 | 11 | 40178082 | C | A |
| 1039 | LRRC4C | rs12575363 | 11 | 40185226 | G | A |
| 1040 | LRRC4C | rs2123267 | 11 | 40201926 | C | T |
| 1041 | LRRC4C | rs7933522 | 11 | 40203677 | T | C |
| 1042 | LRRC4C | rs7125624 | 11 | 40224672 | T | C |
| 1043 | LRRC4C | rs1452465 | 11 | 40231508 | G | A |
| 1044 | LRRC4C | rs1037412 | 11 | 40233300 | C | T |
| 1045 | LRRC4C | rs999004 | 11 | 40234463 | T | C |
| 1046 | LRRC4C | rs1020580 | 11 | 40236938 | A | G |
| 1047 | LRRC4C | rs1020579 | 11 | 40237011 | T | C |
| 1048 | LRRC4C | rs12577986 | 11 | 40252114 | G | A |
| 1049 | LRRC4C | rs1901583 | 11 | 40257768 | G | A |
| 1050 | LRRC4C | rs998447 | 11 | 40267364 | A | C |
| 1051 | MAPK1 | rs2298432 | 22 | 20453188 | C | A |
| 1052 | MAPK1 | rs743409 | 22 | 20459214 | C | T |
| 1053 | MAPK1 | rs2283792 | 22 | 20461124 | G | T |
| 1054 | MAPK1 | rs5749939 | 22 | 20471824 | C | T |
| 1055 | MAPK1 | rs5999515 | 22 | 20475100 | C | T |
| 1056 | MAPK1 | rs2266967 | 22 | 20486788 | A | C |
| 1057 | MAPK1 | rs5749998 | 22 | 20492373 | T | A |
| 1058 | MAPK1 | rs1892846 | 22 | 20516478 | C | A |
| 1059 | MAPK1 | rs8141815 | 22 | 20521584 | G | A |
| 1060 | MAPK1 | rs12172554 | 22 | 20525074 | C | T |
| 1061 | MAPK1 | rs9610417 | 22 | 20529969 | C | T |
| 1062 | MAPK1 | rs8136867 | 22 | 20534792 | A | G |
| 1063 | MAPK1 | rs2876981 | 22 | 20535610 | A | C |
| 1064 | MAPK1 | rs5750113 | 22 | 20550073 | T | C |
| 1065 | MAPK3 | rs11865086 | 16 | 30037993 | C | A |
| 1066 | MASA | rs8732 | 4 | 83570844 | A | G |
| 1067 | MASA | rs10516674 | 4 | 83577785 | A | G |
| 1068 | MASA | rs1878494 | 4 | 83581215 | A | G |
| 1069 | MASA | rs4285076 | 4 | 83588310 | G | A |
| 1070 | MASA | rs1980187 | 4 | 83597895 | A | C |
| 1071 | MASA | rs11929894 | 4 | 83598849 | C | T |
| 1072 | MASA | rs3796472 | 4 | 83599237 | A | G |
| 1073 | MET | rs38840 | 7 | 116100221 | C | T |
| 1074 | MET | rs714180 | 7 | 116106237 | G | A |
| 1075 | MET | rs38841 | 7 | 116107161 | A | G |
| 1076 | MET | rs38845 | 7 | 116109037 | G | A |
| 1077 | MET | rs10487353 | 7 | 116111171 | G | T |
| 1078 | MET | rs722134 | 7 | 116120944 | T | C |
| 1079 | MET | rs2237708 | 7 | 116124115 | T | C |
| 1080 | MET | rs2237710 | 7 | 116124587 | T | G |
| 1081 | MET | rs2237712 | 7 | 116125893 | A | G |
| 1082 | MET | rs2299436 | 7 | 116128618 | A | G |
| 1083 | MET | rs2299437 | 7 | 116128723 | G | A |
| 1084 | MET | rs2237713 | 7 | 116130375 | G | A |
| 1085 | MET | rs17138948 | 7 | 116131545 | C | T |
| 1086 | MET | rs10243024 | 7 | 116133838 | G | A |
| 1087 | MET | rs38855 | 7 | 116145279 | A | G |
| 1088 | MET | rs38857 | 7 | 116152648 | C | T |
| 1089 | MET | rs38859 | 7 | 116166329 | C | T |
| 1090 | MET | rs2237717 | 7 | 116192622 | C | T |
| 1091 | MET | rs2299439 | 7 | 116203245 | T | G |
| 1092 | MET | rs2299440 | 7 | 116203562 | C | T |
| 1093 | MET | rs3807997 | 7 | 116207793 | C | A |
| 1094 | MET | rs10435378 | 7 | 116212728 | A | G |
| 1095 | MET | rs2402118 | 7 | 116215808 | C | A |
| 1096 | MET | rs193688 | 7 | 116220762 | T | C |
| 1097 | MET | rs183642 | 7 | 116220865 | G | A |
| 1098 | MET | rs41736 | 7 | 116223003 | C | T |
| 1099 | MET | rs2023748 | 7 | 116223257 | G | A |
| 1100 | MET | rs1621 | 7 | 116224841 | A | G |
| 1101 | MET | rs6566 | 7 | 116225653 | G | A |
| 1102 | MET | rs41741 | 7 | 116225746 | T | G |
| 1103 | MRAS | rs1199334 | 3 | 139573829 | C | T |
| 1104 | MRAS | rs9289559 | 3 | 139573878 | G | A |
| 1105 | MRAS | rs1199333 | 3 | 139574390 | A | C |
| 1106 | MRAS | rs1720819 | 3 | 139583228 | A | C |
| 1107 | MRAS | rs1209710 | 3 | 139589387 | A | G |
| 1108 | MRAS | rs2306374 | 3 | 139602641 | T | C |
| 1109 | MRAS | rs1199332 | 3 | 139604381 | C | G |
| 1110 | MRAS | rs9818870 | 3 | 139604811 | C | T |
| 1111 | NCK1 | rs9846691 | 3 | 138088960 | C | T |
| 1112 | NCK1 | rs7648817 | 3 | 138132152 | T | C |
| 1113 | NCK1 | rs9833952 | 3 | 138145292 | A | G |
| 1114 | NCK1 | rs1433318 | 3 | 138149143 | T | C |
| 1115 | NCK1 | rs1433319 | 3 | 138149288 | G | A |
| 1116 | NCK2 | rs997136 | 2 | 105732453 | C | T |
| 1117 | NCK2 | rs997522 | 2 | 105732663 | A | G |
| 1118 | NCK2 | rs12987484 | 2 | 105733992 | T | C |
| 1119 | NCK2 | rs10496402 | 2 | 105737744 | A | G |
| 1120 | NCK2 | rs12712216 | 2 | 105739102 | G | A |
| 1121 | NCK2 | rs4851843 | 2 | 105744772 | T | C |
| 1122 | NCK2 | rs12470376 | 2 | 105762809 | C | T |
| 1123 | NCK2 | rs1897169 | 2 | 105764055 | T | C |
| 1124 | NCK2 | rs7588033 | 2 | 105774664 | C | T |
| 1125 | NCK2 | rs2047699 | 2 | 105779508 | G | A |
| 1126 | NCK2 | rs12464067 | 2 | 105782543 | A | G |
| 1127 | NCK2 | rs1367406 | 2 | 105786834 | A | G |
| 1128 | NCK2 | rs6747023 | 2 | 105798287 | A | G |
| 1129 | NCK2 | rs4851870 | 2 | 105803687 | C | T |
| 1130 | NCK2 | rs1465639 | 2 | 105805612 | A | G |
| 1131 | NCK2 | rs2163350 | 2 | 105811712 | C | T |
| 1132 | NCK2 | rs7589561 | 2 | 105820516 | T | C |
| 1133 | NCK2 | rs2377339 | 2 | 105823722 | A | G |
| 1134 | NCK2 | rs6543342 | 2 | 105828687 | C | T |
| 1135 | NCK2 | rs10180659 | 2 | 105831804 | C | T |
| 1136 | NCK2 | rs6745887 | 2 | 105834470 | G | A |
| 1137 | NCK2 | rs3769504 | 2 | 105843523 | C | T |
| 1138 | NCK2 | rs3754806 | 2 | 105853813 | T | C |
| 1139 | NCK2 | rs879900 | 2 | 105860778 | T | C |
| 1140 | NCK2 | rs2033008 | 2 | 105869016 | T | A |
| 1141 | NCK2 | rs6740343 | 2 | 105871909 | G | A |
| 1142 | NFAT5 | rs2161630 | 16 | 68163961 | G | A |
| 1143 | NFAT5 | rs244418 | 16 | 68180262 | G | A |
| 1144 | NFAT5 | rs12447326 | 16 | 68188353 | C | T |
| 1145 | NFAT5 | rs33063 | 16 | 68197717 | G | A |
| 1146 | NFAT5 | rs3848278 | 16 | 68198367 | T | C |
| 1147 | NFAT5 | rs9936464 | 16 | 68201549 | C | A |
| 1148 | NFAT5 | rs7187634 | 16 | 68220871 | T | C |
| 1149 | NFAT5 | rs244415 | 16 | 68224183 | G | A |
| 1150 | NFAT5 | rs9924271 | 16 | 68237721 | A | G |
| 1151 | NFAT5 | rs6499240 | 16 | 68244412 | G | A |
| 1152 | NFAT5 | rs9788837 | 16 | 68245913 | A | G |
| 1153 | NFAT5 | rs920191 | 16 | 68246620 | G | A |
| 1154 | NFAT5 | rs1437137 | 16 | 68262238 | G | A |
| 1155 | NFAT5 | rs3743673 | 16 | 68268811 | A | T |
| 1156 | NFAT5 | rs1064825 | 16 | 68281720 | G | A |
| 1157 | NFAT5 | rs3826154 | 16 | 68288055 | A | G |
| 1158 | NFAT5 | rs7359336 | 16 | 68290960 | A | G |
| 1159 | NFAT5 | rs7359387 | 16 | 68291165 | T | G |
| 1160 | NFAT5 | rs11641233 | 16 | 68292063 | C | T |
| 1161 | NFAT5 | rs12232410 | 16 | 68294351 | G | A |
| 1162 | NFATC1 | rs9962479 | 18 | 75256171 | G | A |
| 1163 | NFATC1 | rs2850723 | 18 | 75258204 | C | T |
| 1164 | NFATC1 | rs549752 | 18 | 75259212 | T | C |
| 1165 | NFATC1 | rs2958431 | 18 | 75261955 | C | T |
| 1166 | NFATC1 | rs643705 | 18 | 75262658 | G | C |
| 1167 | NFATC1 | rs2850724 | 18 | 75263700 | G | A |
| 1168 | NFATC1 | rs8090864 | 18 | 75265043 | A | G |
| 1169 | NFATC1 | rs1562724 | 18 | 75270558 | C | T |
| 1170 | NFATC1 | rs2230112 | 18 | 75272048 | G | T |
| 1171 | NFATC1 | rs1017860 | 18 | 75273193 | C | T |
| 1172 | NFATC1 | rs2002311 | 18 | 75274966 | T | C |
| 1173 | NFATC1 | rs4799055 | 18 | 75282990 | G | T |
| 1174 | NFATC1 | rs1562722 | 18 | 75288152 | A | G |
| 1175 | NFATC1 | rs11664725 | 18 | 75293103 | A | G |
| 1176 | NFATC1 | rs8097131 | 18 | 75293970 | C | A |
| 1177 | NFATC1 | rs8097537 | 18 | 75294233 | C | T |
| 1178 | NFATC1 | rs3826567 | 18 | 75300465 | C | T |
| 1179 | NFATC1 | rs306872 | 18 | 75301832 | T | C |
| 1180 | NFATC1 | rs3786194 | 18 | 75308111 | A | G |
| 1181 | NFATC1 | rs2290154 | 18 | 75312162 | T | C |
| 1182 | NFATC1 | rs9966033 | 18 | 75312751 | T | C |
| 1183 | NFATC1 | rs8090560 | 18 | 75314142 | G | A |
| 1184 | NFATC1 | rs7236492 | 18 | 75321603 | C | T |
| 1185 | NFATC1 | rs8090312 | 18 | 75326141 | A | G |
| 1186 | NFATC1 | rs7239147 | 18 | 75326899 | C | T |
| 1187 | NFATC1 | rs754505 | 18 | 75327355 | G | A |
| 1188 | NFATC1 | rs7227107 | 18 | 75328463 | A | G |
| 1189 | NFATC1 | rs11660128 | 18 | 75328937 | G | T |
| 1190 | NFATC1 | rs160189 | 18 | 75333696 | A | G |
| 1191 | NFATC1 | rs1667667 | 18 | 75345987 | A | G |
| 1192 | NFATC1 | rs754093 | 18 | 75347393 | T | G |
| 1193 | NFATC1 | rs396127 | 18 | 75351625 | T | C |
| 1194 | NFATC1 | rs1660154 | 18 | 75357049 | A | G |
| 1195 | NFATC1 | rs1667676 | 18 | 75357607 | C | T |
| 1196 | NFATC1 | rs1660153 | 18 | 75357928 | C | G |
| 1197 | NFATC1 | rs1660152 | 18 | 75357959 | C | G |
| 1198 | NFATC1 | rs409329 | 18 | 75358390 | A | G |
| 1199 | NFATC1 | rs8099113 | 18 | 75367141 | G | A |
| 1200 | NFATC1 | rs1078633 | 18 | 75369497 | G | T |
| 1201 | NFATC1 | rs372741 | 18 | 75370276 | T | C |
| 1202 | NFATC1 | rs177820 | 18 | 75377951 | T | C |
| 1203 | NFATC1 | rs2044750 | 18 | 75380737 | G | A |
| 1204 | NFATC1 | rs9518 | 18 | 75389793 | T | C |
| 1205 | NFATC2 | rs959996 | 20 | 49442815 | C | T |
| 1206 | NFATC2 | rs6021183 | 20 | 49455555 | T | C |
| 1207 | NFATC2 | rs2426302 | 20 | 49458143 | A | C |
| 1208 | NFATC2 | rs6021193 | 20 | 49470115 | A | G |
| 1209 | NFATC2 | rs6126229 | 20 | 49479043 | T | C |
| 1210 | NFATC2 | rs6013184 | 20 | 49480504 | G | A |
| 1211 | NFATC2 | rs4811174 | 20 | 49481539 | C | T |
| 1212 | NFATC2 | rs2244571 | 20 | 49482965 | A | G |
| 1213 | NFATC2 | rs6096431 | 20 | 49486047 | T | G |
| 1214 | NFATC2 | rs880324 | 20 | 49487596 | G | A |
| 1215 | NFATC2 | rs228832 | 20 | 49493554 | C | T |
| 1216 | NFATC2 | rs228835 | 20 | 49497652 | T | C |
| 1217 | NFATC2 | rs228838 | 20 | 49502107 | C | T |
| 1218 | NFATC2 | rs228840 | 20 | 49504664 | G | A |
| 1219 | NFATC2 | rs6091326 | 20 | 49514748 | C | T |
| 1220 | NFATC2 | rs228851 | 20 | 49518711 | T | G |
| 1221 | NFATC2 | rs8119860 | 20 | 49520169 | G | T |
| 1222 | NFATC2 | rs6013193 | 20 | 49525433 | T | G |
| 1223 | NFATC2 | rs6021231 | 20 | 49525693 | T | C |
| 1224 | NFATC2 | rs6021247 | 20 | 49542386 | A | G |
| 1225 | NFATC2 | rs6123045 | 20 | 49552248 | C | T |
| 1226 | NFATC2 | rs4811184 | 20 | 49558077 | T | C |
| 1227 | NFATC2 | rs6067804 | 20 | 49565470 | G | A |
| 1228 | NFATC2 | rs2273642 | 20 | 49566495 | G | A |
| 1229 | NFATC2 | rs6021264 | 20 | 49568089 | A | G |
| 1230 | NFATC2 | rs6067805 | 20 | 49568497 | T | C |
| 1231 | NFATC2 | rs6021266 | 20 | 49572653 | A | G |
| 1232 | NFATC2 | rs6021275 | 20 | 49588530 | A | G |
| 1233 | NFATC3 | rs3785109 | 16 | 66675811 | G | A |
| 1234 | NFATC3 | rs9932251 | 16 | 66689232 | G | A |
| 1235 | NFATC3 | rs2418736 | 16 | 66712362 | C | T |
| 1236 | NFATC3 | rs3815173 | 16 | 66717789 | A | C |
| 1237 | NFATC3 | rs7193701 | 16 | 66742660 | G | A |
| 1238 | NFATC3 | rs8044995 | 16 | 66746840 | G | A |
| 1239 | NFATC3 | rs4359427 | 16 | 66760247 | C | T |
| 1240 | NFATC3 | rs8046355 | 16 | 66762714 | T | A |
| 1241 | NFATC3 | rs7190307 | 16 | 66775836 | G | A |
| 1242 | NFATC3 | rs7199588 | 16 | 66782082 | C | T |
| 1243 | NFATC3 | rs12598 | 16 | 66783015 | G | A |
| 1244 | NFATC3 | rs1125332 | 16 | 66785280 | T | C |
| 1245 | NFATC3 | rs9938020 | 16 | 66809284 | C | G |
| 1246 | NFATC4 | rs1955915 | 14 | 23908460 | G | A |
| 1247 | NFATC4 | rs2228233 | 14 | 23909004 | C | T |
| 1248 | NFATC4 | rs2295298 | 14 | 23913459 | G | A |
| 1249 | NFATC4 | rs11628929 | 14 | 23913893 | G | A |
| 1250 | NFATC4 | rs3742517 | 14 | 23917790 | T | C |
| 1251 | NGEF | rs1083522 | 2 | 233453764 | G | A |
| 1252 | NGEF | rs2292724 | 2 | 233457921 | T | G |
| 1253 | NGEF | rs2292725 | 2 | 233458317 | C | T |
| 1254 | NGEF | rs11694000 | 2 | 233458910 | A | G |
| 1255 | NGEF | rs2166441 | 2 | 233465686 | A | G |
| 1256 | NGEF | rs748002 | 2 | 233465922 | G | A |
| 1257 | NGEF | rs938569 | 2 | 233467938 | G | C |
| 1258 | NGEF | rs7355380 | 2 | 233480543 | C | T |
| 1259 | NGEF | rs778356 | 2 | 233492961 | G | A |
| 1260 | NGEF | rs2004203 | 2 | 233498690 | G | A |
| 1261 | NGEF | rs1878288 | 2 | 233499633 | C | T |
| 1262 | NGEF | rs1878289 | 2 | 233499851 | C | G |
| 1263 | NGEF | rs11685503 | 2 | 233501760 | A | G |
| 1264 | NGEF | rs6437075 | 2 | 233507643 | G | A |
| 1265 | NGEF | rs4973578 | 2 | 233517487 | C | A |
| 1266 | NGEF | rs6751590 | 2 | 233523121 | C | A |
| 1267 | NGEF | rs6707071 | 2 | 233526574 | G | A |
| 1268 | NGEF | rs2176308 | 2 | 233532446 | G | A |
| 1269 | NGEF | rs2344968 | 2 | 233535030 | G | A |
| 1270 | NGEF | rs11678490 | 2 | 233539193 | G | A |
| 1271 | NGEF | rs4973588 | 2 | 233543218 | G | A |
| 1272 | NGEF | rs10933421 | 2 | 233546271 | T | C |
| 1273 | NGEF | rs3817321 | 2 | 233547295 | C | T |
| 1274 | NGEF | rs6437079 | 2 | 233547417 | A | G |
| 1275 | NGEF | rs6760133 | 2 | 233553748 | T | G |
| 1276 | NGEF | rs11885626 | 2 | 233562640 | A | G |
| 1277 | NGEF | rs6437080 | 2 | 233563704 | G | A |
| 1278 | NGEF | rs4973062 | 2 | 233566879 | T | C |
| 1279 | NGEF | rs3811588 | 2 | 233584218 | C | T |
| 1280 | NGEF | rs6725244 | 2 | 233585308 | C | T |
| 1281 | NGEF | rs6718480 | 2 | 233587309 | A | G |
| 1282 | NRAS | rs14804 | 1 | 115051365 | C | T |
| 1283 | NRAS | rs969273 | 1 | 115058191 | T | C |
| 1284 | NRAS | rs8453 | 1 | 115061121 | C | A |
| 1285 | NRP1 | rs1044268 | 10 | 33506571 | G | A |
| 1286 | NRP1 | rs10080 | 10 | 33507113 | C | T |
| 1287 | NRP1 | rs10827206 | 10 | 33507317 | C | T |
| 1288 | NRP1 | rs1044210 | 10 | 33507326 | C | A |
| 1289 | NRP1 | rs2506140 | 10 | 33507544 | A | G |
| 1290 | NRP1 | rs2506141 | 10 | 33508019 | T | C |
| 1291 | NRP1 | rs2506143 | 10 | 33508174 | A | G |
| 1292 | NRP1 | rs2506144 | 10 | 33508461 | C | T |
| 1293 | NRP1 | rs1048804 | 10 | 33509186 | T | C |
| 1294 | NRP1 | rs2506145 | 10 | 33511135 | T | C |
| 1295 | NRP1 | rs2474729 | 10 | 33513665 | C | T |
| 1296 | NRP1 | rs2228638 | 10 | 33515287 | G | A |
| 1297 | NRP1 | rs734186 | 10 | 33524834 | C | T |
| 1298 | NRP1 | rs1555320 | 10 | 33530024 | C | A |
| 1299 | NRP1 | rs1555319 | 10 | 33530168 | G | C |
| 1300 | NRP1 | rs2474714 | 10 | 33535988 | T | C |
| 1301 | NRP1 | rs2254826 | 10 | 33544470 | C | A |
| 1302 | NRP1 | rs2269101 | 10 | 33546184 | G | A |
| 1303 | NRP1 | rs2269100 | 10 | 33546401 | A | C |
| 1304 | NRP1 | rs1411923 | 10 | 33546783 | C | T |
| 1305 | NRP1 | rs1360456 | 10 | 33546933 | G | A |
| 1306 | NRP1 | rs2269096 | 10 | 33548147 | G | A |
| 1307 | NRP1 | rs4934837 | 10 | 33550619 | G | A |
| 1308 | NRP1 | rs2273466 | 10 | 33551052 | A | G |
| 1309 | NRP1 | rs1331317 | 10 | 33557652 | T | C |
| 1310 | NRP1 | rs3904032 | 10 | 33560273 | G | A |
| 1311 | NRP1 | rs927099 | 10 | 33565841 | A | G |
| 1312 | NRP1 | rs10490939 | 10 | 33574885 | C | T |
| 1313 | NRP1 | rs1888689 | 10 | 33575891 | T | G |
| 1314 | NRP1 | rs2383987 | 10 | 33579333 | T | C |
| 1315 | NRP1 | rs4934583 | 10 | 33583707 | A | G |
| 1316 | NRP1 | rs1319013 | 10 | 33583934 | C | A |
| 1317 | NRP1 | rs3780869 | 10 | 33587470 | G | A |
| 1318 | NRP1 | rs3780867 | 10 | 33587814 | C | T |
| 1319 | NRP1 | rs10490938 | 10 | 33588381 | G | A |
| 1320 | NRP1 | rs2070296 | 10 | 33592700 | G | A |
| 1321 | NRP1 | rs2073320 | 10 | 33593262 | C | T |
| 1322 | NRP1 | rs2243668 | 10 | 33603898 | G | A |
| 1323 | NRP1 | rs12774340 | 10 | 33606941 | T | C |
| 1324 | NRP1 | rs1952983 | 10 | 33608940 | A | C |
| 1325 | NRP1 | rs869636 | 10 | 33612044 | T | C |
| 1326 | NRP1 | rs7079372 | 10 | 33613811 | C | T |
| 1327 | NRP1 | rs2776928 | 10 | 33614410 | T | C |
| 1328 | NRP1 | rs1331326 | 10 | 33619895 | A | G |
| 1329 | NRP1 | rs6481844 | 10 | 33624136 | T | C |
| 1330 | NRP1 | rs2776932 | 10 | 33632411 | T | C |
| 1331 | NRP1 | rs2065364 | 10 | 33634007 | G | A |
| 1332 | NRP1 | rs2804492 | 10 | 33642051 | C | T |
| 1333 | NRP1 | rs2776937 | 10 | 33646194 | G | A |
| 1334 | NRP1 | rs4934597 | 10 | 33654726 | C | T |
| 1335 | NRP1 | rs1360457 | 10 | 33656934 | C | T |
| 1336 | NRP1 | rs2026319 | 10 | 33661507 | G | C |
| 1337 | NRP1 | rs1888684 | 10 | 33662982 | C | T |
| 1338 | NTN1 | rs940853 | 17 | 8867898 | T | C |
| 1339 | NTN1 | rs1473664 | 17 | 8869135 | C | A |
| 1340 | NTN1 | rs1880646 | 17 | 8870569 | T | C |
| 1341 | NTN1 | rs4791331 | 17 | 8872806 | C | T |
| 1342 | NTN1 | rs4791774 | 17 | 8872843 | A | G |
| 1343 | NTN1 | rs7223172 | 17 | 8873612 | C | A |
| 1344 | NTN1 | rs940854 | 17 | 8875487 | T | C |
| 1345 | NTN1 | rs9674710 | 17 | 8881933 | G | A |
| 1346 | NTN1 | rs10521156 | 17 | 8883454 | C | T |
| 1347 | NTN1 | rs8076457 | 17 | 8884653 | C | T |
| 1348 | NTN1 | rs9915089 | 17 | 8893618 | C | T |
| 1349 | NTN1 | rs8069536 | 17 | 8897009 | G | T |
| 1350 | NTN1 | rs1525758 | 17 | 8901607 | G | A |
| 1351 | NTN1 | rs8081823 | 17 | 8906275 | G | A |
| 1352 | NTN1 | rs2030795 | 17 | 8908000 | G | A |
| 1353 | NTN1 | rs7222847 | 17 | 8908104 | T | C |
| 1354 | NTN1 | rs12948329 | 17 | 8910381 | A | G |
| 1355 | NTN1 | rs10852910 | 17 | 8914276 | G | A |
| 1356 | NTN1 | rs8079384 | 17 | 8922856 | A | G |
| 1357 | NTN1 | rs869019 | 17 | 8925626 | G | A |
| 1358 | NTN1 | rs2030793 | 17 | 8931625 | G | T |
| 1359 | NTN1 | rs1568901 | 17 | 8933806 | A | G |
| 1360 | NTN1 | rs871827 | 17 | 8934625 | T | G |
| 1361 | NTN1 | rs7207041 | 17 | 8936270 | C | A |
| 1362 | NTN1 | rs9893768 | 17 | 8942955 | A | C |
| 1363 | NTN1 | rs4791779 | 17 | 8946117 | G | A |
| 1364 | NTN1 | rs9914560 | 17 | 8951167 | T | C |
| 1365 | NTN1 | rs2091317 | 17 | 8953425 | C | T |
| 1366 | NTN1 | rs4791334 | 17 | 8957732 | T | C |
| 1367 | NTN1 | rs4641781 | 17 | 8966801 | G | A |
| 1368 | NTN1 | rs4791794 | 17 | 8967534 | C | T |
| 1369 | NTN1 | rs4791337 | 17 | 8975449 | G | A |
| 1370 | NTN1 | rs4791798 | 17 | 8987680 | G | A |
| 1371 | NTN1 | rs1860283 | 17 | 8992041 | C | T |
| 1372 | NTN1 | rs3826464 | 17 | 8998633 | G | A |
| 1373 | NTN1 | rs726598 | 17 | 9000212 | G | A |
| 1374 | NTN1 | rs2072321 | 17 | 9006725 | C | T |
| 1375 | NTN1 | rs2286511 | 17 | 9006978 | T | C |
| 1376 | NTN1 | rs9901968 | 17 | 9011370 | G | A |
| 1377 | NTN1 | rs1034893 | 17 | 9013279 | A | G |
| 1378 | NTN1 | rs758413 | 17 | 9014434 | C | T |
| 1379 | NTN1 | rs3785975 | 17 | 9019051 | C | T |
| 1380 | NTN1 | rs3785977 | 17 | 9021295 | G | A |
| 1381 | NTN1 | rs2302196 | 17 | 9026952 | C | A |
| 1382 | NTN1 | rs3785982 | 17 | 9030948 | C | T |
| 1383 | NTN1 | rs6503186 | 17 | 9032985 | T | C |
| 1384 | NTN1 | rs7225607 | 17 | 9036406 | G | A |
| 1385 | NTN1 | rs8081873 | 17 | 9038657 | T | G |
| 1386 | NTN1 | rs740642 | 17 | 9042893 | C | T |
| 1387 | NTN1 | rs3785983 | 17 | 9048958 | T | C |
| 1388 | NTN1 | rs7224773 | 17 | 9053827 | G | T |
| 1389 | NTN1 | rs718097 | 17 | 9056129 | A | G |
| 1390 | NTN1 | rs9899990 | 17 | 9062303 | G | A |
| 1391 | NTN1 | rs9897341 | 17 | 9064618 | T | C |
| 1392 | NTN1 | rs1009675 | 17 | 9065602 | T | G |
| 1393 | NTN1 | rs3785988 | 17 | 9065629 | G | A |
| 1394 | NTN1 | rs12939263 | 17 | 9067734 | C | T |
| 1395 | NTN1 | rs3785991 | 17 | 9071042 | G | A |
| 1396 | NTN1 | rs3785996 | 17 | 9073585 | G | A |
| 1397 | NTN1 | rs11652483 | 17 | 9075421 | G | A |
| 1398 | NTN1 | rs3786003 | 17 | 9077877 | G | A |
| 1399 | NTN1 | rs9909240 | 17 | 9080815 | A | G |
| 1400 | NTN1 | rs3744656 | 17 | 9082047 | T | G |
| 1401 | NTN4 | rs8699 | 12 | 94576013 | T | C |
| 1402 | NTN4 | rs1362970 | 12 | 94576656 | G | T |
| 1403 | NTN4 | rs1052651 | 12 | 94576851 | C | T |
| 1404 | NTN4 | rs2367563 | 12 | 94581314 | T | C |
| 1405 | NTN4 | rs1421436 | 12 | 94583258 | C | T |
| 1406 | NTN4 | rs2367558 | 12 | 94590292 | G | A |
| 1407 | NTN4 | rs728661 | 12 | 94595450 | T | C |
| 1408 | NTN4 | rs11108193 | 12 | 94598453 | C | T |
| 1409 | NTN4 | rs1362971 | 12 | 94599653 | T | C |
| 1410 | NTN4 | rs2431014 | 12 | 94608310 | T | C |
| 1411 | NTN4 | rs2468366 | 12 | 94611999 | G | A |
| 1412 | NTN4 | rs1362966 | 12 | 94617488 | C | A |
| 1413 | NTN4 | rs10507058 | 12 | 94622877 | C | T |
| 1414 | NTN4 | rs2468358 | 12 | 94623606 | A | G |
| 1415 | NTN4 | rs11108203 | 12 | 94624724 | C | T |
| 1416 | NTN4 | rs10507059 | 12 | 94629964 | C | T |
| 1417 | NTN4 | rs4129599 | 12 | 94631073 | A | G |
| 1418 | NTN4 | rs10777720 | 12 | 94634521 | C | T |
| 1419 | NTN4 | rs4128893 | 12 | 94647739 | C | T |
| 1420 | NTN4 | rs7137835 | 12 | 94654537 | C | T |
| 1421 | NTN4 | rs11108225 | 12 | 94654843 | C | T |
| 1422 | NTN4 | rs10444523 | 12 | 94656230 | A | G |
| 1423 | NTN4 | rs11108230 | 12 | 94658784 | T | C |
| 1424 | NTN4 | rs7310388 | 12 | 94679759 | C | T |
| 1425 | NTN4 | rs10735333 | 12 | 94682958 | G | A |
| 1426 | NTN4 | rs4762617 | 12 | 94686198 | A | C |
| 1427 | NTN4 | rs4237913 | 12 | 94686418 | T | C |
| 1428 | NTN4 | rs4533108 | 12 | 94692535 | A | G |
| 1429 | NTN4 | rs7975425 | 12 | 94705995 | G | A |
| 1430 | NTN4 | rs7139030 | 12 | 94709442 | G | A |
| 1431 | NTN4 | rs2117915 | 12 | 94709872 | T | C |
| 1432 | NTNG1 | rs4481881 | 1 | 107487940 | T | C |
| 1433 | NTNG1 | rs4307594 | 1 | 107494689 | T | C |
| 1434 | NTNG1 | rs12743478 | 1 | 107502628 | C | T |
| 1435 | NTNG1 | rs4364907 | 1 | 107505395 | T | C |
| 1436 | NTNG1 | rs9628993 | 1 | 107511242 | A | G |
| 1437 | NTNG1 | rs10494061 | 1 | 107511900 | C | T |
| 1438 | NTNG1 | rs4385741 | 1 | 107525841 | T | G |
| 1439 | NTNG1 | rs10748503 | 1 | 107570350 | A | C |
| 1440 | NTNG1 | rs5012609 | 1 | 107576900 | G | A |
| 1441 | NTNG1 | rs12146111 | 1 | 107586886 | C | T |
| 1442 | NTNG1 | rs10494066 | 1 | 107591030 | A | G |
| 1443 | NTNG1 | rs3123373 | 1 | 107616649 | G | T |
| 1444 | NTNG1 | rs4244126 | 1 | 107618273 | A | G |
| 1445 | NTNG1 | rs12133394 | 1 | 107619072 | C | T |
| 1446 | NTNG1 | rs3125673 | 1 | 107623707 | G | A |
| 1447 | NTNG1 | rs3123376 | 1 | 107627388 | A | G |
| 1448 | NTNG1 | rs10494067 | 1 | 107632380 | A | C |
| 1449 | NTNG1 | rs4604720 | 1 | 107638681 | A | G |
| 1450 | NTNG1 | rs4132604 | 1 | 107644361 | G | T |
| 1451 | NTNG1 | rs10494068 | 1 | 107646032 | G | A |
| 1452 | NTNG1 | rs4244127 | 1 | 107650562 | A | G |
| 1453 | NTNG1 | rs6692538 | 1 | 107657725 | G | A |
| 1454 | NTNG1 | rs7411507 | 1 | 107667500 | A | G |
| 1455 | NTNG1 | rs7531522 | 1 | 107672804 | C | G |
| 1456 | NTNG1 | rs6670042 | 1 | 107676298 | A | G |
| 1457 | NTNG1 | rs6672549 | 1 | 107676481 | A | G |
| 1458 | NTNG1 | rs12066815 | 1 | 107684565 | A | G |
| 1459 | NTNG1 | rs7555385 | 1 | 107691170 | T | G |
| 1460 | NTNG1 | rs6664221 | 1 | 107711400 | T | C |
| 1461 | NTNG1 | rs11185100 | 1 | 107719841 | T | G |
| 1462 | NTNG1 | rs6662274 | 1 | 107720888 | C | A |
| 1463 | NTNG1 | rs12736002 | 1 | 107725672 | T | C |
| 1464 | NTNG1 | rs6681988 | 1 | 107731887 | T | C |
| 1465 | NTNG1 | rs746603 | 1 | 107732682 | T | C |
| 1466 | NTNG1 | rs10494069 | 1 | 107736168 | A | G |
| 1467 | NTNG1 | rs2587895 | 1 | 107737915 | C | T |
| 1468 | NTNG1 | rs920835 | 1 | 107748324 | G | A |
| 1469 | NTNG1 | rs2218404 | 1 | 107754127 | G | T |
| 1470 | NTNG1 | rs1444040 | 1 | 107766001 | G | C |
| 1471 | NTNG1 | rs600647 | 1 | 107773543 | G | A |
| 1472 | NTNG1 | rs480392 | 1 | 107780918 | C | — |
| 1473 | NTNG1 | rs483206 | 1 | 107781201 | G | T |
| 1474 | NTNG1 | rs1444038 | 1 | 107782838 | G | A |
| 1475 | NTNG1 | rs11803905 | 1 | 107790836 | C | T |
| 1476 | NTNG1 | rs640554 | 1 | 107791587 | C | T |
| 1477 | NTNG1 | rs628117 | 1 | 107798628 | A | G |
| 1478 | NTNG1 | rs521721 | 1 | 107802970 | C | T |
| 1479 | NTNG1 | rs977799 | 1 | 107807375 | C | T |
| 1480 | NTNG1 | rs12354155 | 1 | 107812742 | G | A |
| 1481 | NTNG1 | rs3813219 | 1 | 107817853 | T | C |
| 1482 | NTNG1 | rs96501 | 1 | 107820369 | T | C |
| 1483 | NTNG1 | rs594165 | 1 | 107820486 | C | A |
| 1484 | NTNG1 | rs4915045 | 1 | 107825111 | A | C |
| 1485 | NTNG1 | rs597332 | 1 | 107826390 | G | A |
| 1486 | PAK1 | rs2729762 | 11 | 76711346 | T | C |
| 1487 | PAK1 | rs2844337 | 11 | 76711355 | T | G |
| 1488 | PAK1 | rs1377470 | 11 | 76728361 | A | C |
| 1489 | PAK1 | rs51500 | 11 | 76729467 | G | A |
| 1490 | PAK1 | rs2250647 | 11 | 76747393 | T | C |
| 1491 | PAK1 | rs2729760 | 11 | 76766994 | T | C |
| 1492 | PAK1 | rs562939 | 11 | 76767858 | C | T |
| 1493 | PAK1 | rs2298793 | 11 | 76768165 | T | C |
| 1494 | PAK1 | rs2729747 | 11 | 76776126 | A | G |
| 1495 | PAK1 | rs618012 | 11 | 76777464 | C | G |
| 1496 | PAK1 | rs472691 | 11 | 76781645 | G | C |
| 1497 | PAK1 | rs558470 | 11 | 76784347 | G | A |
| 1498 | PAK1 | rs2725830 | 11 | 76819365 | A | G |
| 1499 | PAK1 | rs527589 | 11 | 76832286 | A | G |
| 1500 | PAK1 | rs7109645 | 11 | 76863970 | A | C |
| 1501 | PAK2 | rs1357287 | 3 | 197969363 | C | T |
| 1502 | PAK2 | rs1798619 | 3 | 197970247 | T | G |
| 1503 | PAK2 | rs9872035 | 3 | 197979098 | T | C |
| 1504 | PAK2 | rs2089979 | 3 | 197985809 | A | G |
| 1505 | PAK2 | rs9852432 | 3 | 197991477 | G | A |
| 1506 | PAK2 | rs1718414 | 3 | 197999032 | A | G |
| 1507 | PAK2 | rs7646247 | 3 | 198003605 | C | T |
| 1508 | PAK2 | rs6583176 | 3 | 198009974 | G | A |
| 1509 | PAK2 | rs11185455 | 3 | 198025976 | A | G |
| 1510 | PAK2 | rs2166799 | 3 | 198031934 | T | C |
| 1511 | PAK2 | rs6583177 | 3 | 198034138 | G | T |
| 1512 | PAK2 | rs2084385 | 3 | 198038263 | G | A |
| 1513 | PAK2 | rs9109 | 3 | 198041239 | G | A |
| 1514 | PAK2 | rs15614 | 3 | 198043345 | A | G |
| 1515 | PAK3 | rs1890141 | X | 110232669 | C | — |
| 1516 | PAK3 | rs2050750 | X | 110248151 | T | A |
| 1517 | PAK3 | rs1884552 | X | 110289631 | T | A |
| 1518 | PAK3 | rs9320060 | X | 110300105 | T | C |
| 1519 | PAK3 | rs10521532 | X | 110304158 | A | G |
| 1520 | PAK3 | rs10521533 | X | 110307886 | A | G |
| 1521 | PAK3 | rs10521535 | X | 110346482 | C | G |
| 1522 | PAK4 | rs12609418 | 19 | 44309291 | C | T |
| 1523 | PAK4 | rs692191 | 19 | 44314685 | T | C |
| 1524 | PAK4 | rs11669124 | 19 | 44315091 | G | A |
| 1525 | PAK4 | rs17722300 | 19 | 44322374 | G | A |
| 1526 | PAK4 | rs879689 | 19 | 44342669 | C | T |
| 1527 | PAK4 | rs7257109 | 19 | 44354746 | G | A |
| 1528 | PAK4 | rs11671084 | 19 | 44357079 | G | C |
| 1529 | PAK4 | rs1529712 | 19 | 44358806 | T | C |
| 1530 | PAK6 | rs2306480 | 15 | 38326664 | T | C |
| 1531 | PAK6 | rs2277562 | 15 | 38329490 | C | T |
| 1532 | PAK6 | rs1048163 | 15 | 38330514 | T | C |
| 1533 | PAK6 | rs11636097 | 15 | 38332729 | A | G |
| 1534 | PAK6 | rs2068001 | 15 | 38337440 | G | A |
| 1535 | PAK6 | rs731497 | 15 | 38342356 | A | G |
| 1536 | PAK6 | rs936216 | 15 | 38344559 | T | G |
| 1537 | PAK6 | rs2412504 | 15 | 38345355 | A | G |
| 1538 | PAK6 | rs2016902 | 15 | 38345378 | C | T |
| 1539 | PAK6 | rs936214 | 15 | 38352996 | C | T |
| 1540 | PAK6 | rs2242119 | 15 | 38356114 | G | T |
| 1541 | PAK7 | rs3185693 | 20 | 9467466 | C | A |
| 1542 | PAK7 | rs2072952 | 20 | 9473600 | A | G |
| 1543 | PAK7 | rs6056693 | 20 | 9479739 | A | T |
| 1544 | PAK7 | rs6039498 | 20 | 9480699 | C | T |
| 1545 | PAK7 | rs6039502 | 20 | 9488837 | G | A |
| 1546 | PAK7 | rs2297348 | 20 | 9491832 | G | A |
| 1547 | PAK7 | rs10485733 | 20 | 9492721 | T | C |
| 1548 | PAK7 | rs2026338 | 20 | 9502027 | T | G |
| 1549 | PAK7 | rs2026339 | 20 | 9502124 | T | C |
| 1550 | PAK7 | rs9967942 | 20 | 9503780 | A | C |
| 1551 | PAK7 | rs6056712 | 20 | 9504907 | C | T |
| 1552 | PAK7 | rs3746545 | 20 | 9508711 | T | C |
| 1553 | PAK7 | rs6077567 | 20 | 9517768 | G | A |
| 1554 | PAK7 | rs6056725 | 20 | 9522062 | A | C |
| 1555 | PAK7 | rs6056733 | 20 | 9524642 | A | G |
| 1556 | PAK7 | rs6056740 | 20 | 9529875 | T | C |
| 1557 | PAK7 | rs6140981 | 20 | 9533872 | T | C |
| 1558 | PAK7 | rs928366 | 20 | 9537195 | T | C |
| 1559 | PAK7 | rs2225471 | 20 | 9537576 | T | C |
| 1560 | PAK7 | rs6056755 | 20 | 9540616 | A | G |
| 1561 | PAK7 | rs6039524 | 20 | 9540675 | C | A |
| 1562 | PAK7 | rs7272361 | 20 | 9542880 | T | C |
| 1563 | PAK7 | rs219861 | 20 | 9545554 | A | G |
| 1564 | PAK7 | rs219870 | 20 | 9550874 | T | G |
| 1565 | PAK7 | rs6039529 | 20 | 9556976 | G | A |
| 1566 | PAK7 | rs6086964 | 20 | 9559550 | C | A |
| 1567 | PAK7 | rs12624866 | 20 | 9564074 | A | G |
| 1568 | PAK7 | rs6516479 | 20 | 9566760 | A | G |
| 1569 | PAK7 | rs6056774 | 20 | 9572586 | T | C |
| 1570 | PAK7 | rs10485734 | 20 | 9573786 | G | A |
| 1571 | PAK7 | rs6516482 | 20 | 9575371 | A | G |
| 1572 | PAK7 | rs6086972 | 20 | 9575907 | C | A |
| 1573 | PAK7 | rs6039533 | 20 | 9578907 | G | A |
| 1574 | PAK7 | rs4816158 | 20 | 9580608 | G | A |
| 1575 | PAK7 | rs6039536 | 20 | 9582409 | C | T |
| 1576 | PAK7 | rs6133735 | 20 | 9594833 | C | T |
| 1577 | PAK7 | rs2423401 | 20 | 9597073 | C | T |
| 1578 | PAK7 | rs6118687 | 20 | 9597233 | G | A |
| 1579 | PAK7 | rs2423412 | 20 | 9603983 | G | A |
| 1580 | PAK7 | rs6118695 | 20 | 9604405 | G | A |
| 1581 | PAK7 | rs6141004 | 20 | 9611863 | C | T |
| 1582 | PAK7 | rs6077585 | 20 | 9613894 | T | C |
| 1583 | PAK7 | rs2050104 | 20 | 9619083 | T | C |
| 1584 | PAK7 | rs6056809 | 20 | 9625745 | C | A |
| 1585 | PAK7 | rs6086987 | 20 | 9633154 | A | G |
| 1586 | PAK7 | rs6056812 | 20 | 9633524 | C | T |
| 1587 | PAK7 | rs6086988 | 20 | 9633975 | G | A |
| 1588 | PAK7 | rs722483 | 20 | 9652303 | G | C |
| 1589 | PAK7 | rs2423438 | 20 | 9652942 | T | G |
| 1590 | PAK7 | rs1033470 | 20 | 9673696 | A | G |
| 1591 | PAK7 | rs2423462 | 20 | 9682769 | T | C |
| 1592 | PAK7 | rs1569625 | 20 | 9688440 | T | C |
| 1593 | PAK7 | rs2423467 | 20 | 9689875 | A | G |
| 1594 | PAK7 | rs926502 | 20 | 9692972 | T | G |
| 1595 | PAK7 | rs6141031 | 20 | 9697517 | A | G |
| 1596 | PAK7 | rs6516498 | 20 | 9707413 | G | A |
| 1597 | PAK7 | rs2223569 | 20 | 9712262 | A | C |
| 1598 | PAK7 | rs994487 | 20 | 9716981 | C | T |
| 1599 | PAK7 | rs6141035 | 20 | 9720903 | A | G |
| 1600 | PAK7 | rs10485735 | 20 | 9721876 | G | A |
| 1601 | PAK7 | rs2206481 | 20 | 9723550 | G | T |
| 1602 | PAK7 | rs6087032 | 20 | 9747062 | C | T |
| 1603 | PAK7 | rs6118746 | 20 | 9749669 | T | C |
| 1604 | PAK7 | rs6516503 | 20 | 9752834 | T | C |
| 1605 | PAK7 | rs6056909 | 20 | 9755526 | T | C |
| 1606 | PAK7 | rs221015 | 20 | 9762705 | T | C |
| 1607 | PAK7 | rs221013 | 20 | 9763527 | C | T |
| 1608 | PAK7 | rs761226 | 20 | 9769006 | T | C |
| 1609 | PLXNA1 | rs747967 | 3 | 128203253 | C | A |
| 1610 | PLXNA1 | rs1347003 | 3 | 128214736 | A | G |
| 1611 | PLXNA1 | rs9870165 | 3 | 128214848 | C | A |
| 1612 | PLXNA1 | rs4679323 | 3 | 128219957 | A | C |
| 1613 | PLXNA1 | rs9289290 | 3 | 128223700 | G | A |
| 1614 | PLXNA1 | rs4679325 | 3 | 128224167 | C | T |
| 1615 | PLXNA1 | rs9851451 | 3 | 128229379 | A | G |
| 1616 | PLXNA1 | rs9833827 | 3 | 128229420 | T | G |
| 1617 | PLXNA1 | rs3749395 | 3 | 128237823 | G | A |
| 1618 | PLXNA2 | rs3811386 | 1 | 206271021 | T | G |
| 1619 | PLXNA2 | rs1409831 | 1 | 206276459 | G | T |
| 1620 | PLXNA2 | rs2767566 | 1 | 206279019 | T | C |
| 1621 | PLXNA2 | rs2785622 | 1 | 206281126 | T | C |
| 1622 | PLXNA2 | rs1536471 | 1 | 206285786 | G | T |
| 1623 | PLXNA2 | rs6540451 | 1 | 206291979 | G | A |
| 1624 | PLXNA2 | rs841865 | 1 | 206292531 | G | A |
| 1625 | PLXNA2 | rs753264 | 1 | 206295998 | T | C |
| 1626 | PLXNA2 | rs12057548 | 1 | 206302786 | G | A |
| 1627 | PLXNA2 | rs752016 | 1 | 206304299 | T | C |
| 1628 | PLXNA2 | rs2478816 | 1 | 206311475 | C | T |
| 1629 | PLXNA2 | rs2274446 | 1 | 206319078 | G | A |
| 1630 | PLXNA2 | rs6540453 | 1 | 206322499 | C | T |
| 1631 | PLXNA2 | rs716461 | 1 | 206324251 | G | A |
| 1632 | PLXNA2 | rs2498024 | 1 | 206329772 | G | A |
| 1633 | PLXNA2 | rs2498023 | 1 | 206329844 | T | G |
| 1634 | PLXNA2 | rs2148699 | 1 | 206334200 | A | G |
| 1635 | PLXNA2 | rs3818517 | 1 | 206336866 | T | G |
| 1636 | PLXNA2 | rs12083980 | 1 | 206345499 | C | T |
| 1637 | PLXNA2 | rs11118988 | 1 | 206362705 | A | G |
| 1638 | PLXNA2 | rs6660047 | 1 | 206363720 | G | A |
| 1639 | PLXNA2 | rs681241 | 1 | 206367113 | T | G |
| 1640 | PLXNA2 | rs895241 | 1 | 206369255 | T | C |
| 1641 | PLXNA2 | rs3811390 | 1 | 206370675 | G | A |
| 1642 | PLXNA2 | rs473500 | 1 | 206377531 | C | T |
| 1643 | PLXNA2 | rs668878 | 1 | 206379932 | T | C |
| 1644 | PLXNA2 | rs2007188 | 1 | 206386343 | G | C |
| 1645 | PLXNA2 | rs1875431 | 1 | 206387740 | C | T |
| 1646 | PLXNA2 | rs550948 | 1 | 206394334 | C | T |
| 1647 | PLXNA2 | rs1604364 | 1 | 206396390 | A | G |
| 1648 | PLXNA2 | rs2256712 | 1 | 206404029 | G | A |
| 1649 | PLXNA2 | rs6656560 | 1 | 206427769 | G | A |
| 1650 | PLXNA2 | rs7411544 | 1 | 206428792 | G | T |
| 1651 | PLXNA2 | rs11119011 | 1 | 206440017 | A | G |
| 1652 | PLXNA2 | rs1664232 | 1 | 206448662 | C | T |
| 1653 | PLXNA2 | rs1664227 | 1 | 206456708 | G | C |
| 1654 | PLXNA2 | rs4844657 | 1 | 206456777 | C | T |
| 1655 | PLXNA2 | rs4844658 | 1 | 206456784 | T | C |
| 1656 | PLXNA2 | rs3748735 | 1 | 206457091 | C | T |
| 1657 | PLXNA2 | rs11119014 | 1 | 206457720 | T | C |
| 1658 | PLXNA2 | rs881758 | 1 | 206460170 | C | A |
| 1659 | PLXNA2 | rs3748737 | 1 | 206462778 | T | C |
| 1660 | PLXNA2 | rs1262046 | 1 | 206467114 | T | C |
| 1661 | PLXNA2 | rs2782934 | 1 | 206475125 | A | G |
| 1662 | PLXNA2 | rs6685511 | 1 | 206476782 | G | A |
| 1663 | PLXNA2 | rs1272623 | 1 | 206477257 | A | C |
| 1664 | PLXNA2 | rs1335045 | 1 | 206480655 | T | C |
| 1665 | PLXNA3 | rs5987266 | X | 153349440 | G | C |
| 1666 | PLXNA3 | rs5987267 | X | 153352557 | G | C |
| 1667 | PLXNA3 | rs5945431 | X | 153354909 | A | G |
| 1668 | PLXNB1 | rs9812647 | 3 | 48423917 | C | G |
| 1669 | PLXNB1 | rs11130170 | 3 | 48424900 | C | T |
| 1670 | PLXNB1 | rs10470686 | 3 | 48424959 | C | T |
| 1671 | PLXNB1 | rs2362450 | 3 | 48436316 | C | G |
| 1672 | PLXNB1 | rs6442120 | 3 | 48439506 | C | T |
| 1673 | PLXNC1 | rs4761585 | 12 | 93064805 | T | C |
| 1674 | PLXNC1 | rs4761586 | 12 | 93065045 | A | G |
| 1675 | PLXNC1 | rs2230754 | 12 | 93067636 | C | T |
| 1676 | PLXNC1 | rs7131826 | 12 | 93070450 | T | C |
| 1677 | PLXNC1 | rs10507030 | 12 | 93072090 | A | T |
| 1678 | PLXNC1 | rs4237889 | 12 | 93073104 | G | A |
| 1679 | PLXNC1 | rs3847801 | 12 | 93074138 | T | C |
| 1680 | PLXNC1 | rs11107420 | 12 | 93078943 | G | A |
| 1681 | PLXNC1 | rs3843641 | 12 | 93083377 | C | T |
| 1682 | PLXNC1 | rs2361359 | 12 | 93087005 | G | C |
| 1683 | PLXNC1 | rs2365736 | 12 | 93088048 | T | C |
| 1684 | PLXNC1 | rs3847804 | 12 | 93090428 | A | G |
| 1685 | PLXNC1 | rs4761466 | 12 | 93093490 | G | T |
| 1686 | PLXNC1 | rs4761590 | 12 | 93097941 | T | C |
| 1687 | PLXNC1 | rs3847810 | 12 | 93103155 | A | G |
| 1688 | PLXNC1 | rs3751189 | 12 | 93104416 | A | G |
| 1689 | PLXNC1 | rs769283 | 12 | 93104567 | A | G |
| 1690 | PLXNC1 | rs3847812 | 12 | 93105517 | T | C |
| 1691 | PLXNC1 | rs10777585 | 12 | 93110843 | G | A |
| 1692 | PLXNC1 | rs10745679 | 12 | 93113117 | G | A |
| 1693 | PLXNC1 | rs12425752 | 12 | 93117092 | C | T |
| 1694 | PLXNC1 | rs10507031 | 12 | 93117122 | T | C |
| 1695 | PLXNC1 | rs10859685 | 12 | 93118767 | G | A |
| 1696 | PLXNC1 | rs7296806 | 12 | 93120688 | C | T |
| 1697 | PLXNC1 | rs2305971 | 12 | 93127430 | T | C |
| 1698 | PLXNC1 | rs10507033 | 12 | 93129262 | T | C |
| 1699 | PLXNC1 | rs7298918 | 12 | 93135142 | T | C |
| 1700 | PLXNC1 | rs7969106 | 12 | 93136882 | A | C |
| 1701 | PLXNC1 | rs10507034 | 12 | 93138432 | G | A |
| 1702 | PLXNC1 | rs2291333 | 12 | 93145188 | A | G |
| 1703 | PLXNC1 | rs2291332 | 12 | 93145268 | C | T |
| 1704 | PLXNC1 | rs10745681 | 12 | 93148833 | G | T |
| 1705 | PLXNC1 | rs9943825 | 12 | 93149802 | T | C |
| 1706 | PLXNC1 | rs2361355 | 12 | 93154159 | T | C |
| 1707 | PLXNC1 | rs2230755 | 12 | 93155626 | C | T |
| 1708 | PLXNC1 | rs2291327 | 12 | 93161430 | T | C |
| 1709 | PLXNC1 | rs864106 | 12 | 93161687 | A | G |
| 1710 | PLXNC1 | rs870034 | 12 | 93164225 | C | T |
| 1711 | PLXNC1 | rs2054724 | 12 | 93166995 | G | T |
| 1712 | PLXNC1 | rs6538491 | 12 | 93169385 | G | A |
| 1713 | PLXNC1 | rs2291326 | 12 | 93173075 | A | G |
| 1714 | PLXNC1 | rs865569 | 12 | 93173233 | C | T |
| 1715 | PLXNC1 | rs2242499 | 12 | 93174455 | G | A |
| 1716 | PLXNC1 | rs2306664 | 12 | 93177357 | G | A |
| 1717 | PLXNC1 | rs10507035 | 12 | 93179116 | G | A |
| 1718 | PLXNC1 | rs10507036 | 12 | 93186877 | G | A |
| 1719 | PLXNC1 | rs7970004 | 12 | 93189312 | C | T |
| 1720 | PLXNC1 | rs12579612 | 12 | 93189923 | A | G |
| 1721 | PLXNC1 | rs1175500 | 12 | 93189938 | A | G |
| 1722 | PLXNC1 | rs832503 | 12 | 93198058 | G | A |
| 1723 | PLXNC1 | rs832502 | 12 | 93199272 | T | C |
| 1724 | PLXNC1 | rs10777588 | 12 | 93202502 | C | T |
| 1725 | PLXNC1 | rs7302972 | 12 | 93206737 | G | A |
| 1726 | PLXNC1 | rs4761470 | 12 | 93207334 | G | A |
| 1727 | PLXNC1 | rs10745682 | 12 | 93212040 | C | T |
| 1728 | PLXNC1 | rs17302436 | 12 | 93217381 | T | C |
| 1729 | PPP3CA | rs1042094 | 4 | 102165970 | A | G |
| 1730 | PPP3CA | rs3804350 | 4 | 102166952 | C | T |
| 1731 | PPP3CA | rs2251238 | 4 | 102180969 | C | T |
| 1732 | PPP3CA | rs1358312 | 4 | 102190473 | G | A |
| 1733 | PPP3CA | rs10026659 | 4 | 102194877 | G | A |
| 1734 | PPP3CA | rs17124 | 4 | 102198483 | C | G |
| 1735 | PPP3CA | rs6851231 | 4 | 102202629 | T | G |
| 1736 | PPP3CA | rs10516474 | 4 | 102208838 | G | A |
| 1737 | PPP3CA | rs2695203 | 4 | 102214409 | T | C |
| 1738 | PPP3CA | rs3804357 | 4 | 102221145 | C | A |
| 1739 | PPP3CA | rs9307252 | 4 | 102221388 | C | T |
| 1740 | PPP3CA | rs2732504 | 4 | 102228849 | A | G |
| 1741 | PPP3CA | rs9990461 | 4 | 102238087 | C | T |
| 1742 | PPP3CA | rs9990811 | 4 | 102238273 | G | T |
| 1743 | PPP3CA | rs931870 | 4 | 102247952 | G | C |
| 1744 | PPP3CA | rs2732510 | 4 | 102250797 | A | C |
| 1745 | PPP3CA | rs10516473 | 4 | 102251530 | C | A |
| 1746 | PPP3CA | rs2659546 | 4 | 102256741 | G | A |
| 1747 | PPP3CA | rs6820738 | 4 | 102260258 | G | T |
| 1748 | PPP3CA | rs1506801 | 4 | 102262632 | G | A |
| 1749 | PPP3CA | rs2732514 | 4 | 102277864 | G | A |
| 1750 | PPP3CA | rs963065 | 4 | 102284653 | G | A |
| 1751 | PPP3CA | rs1405688 | 4 | 102293156 | T | C |
| 1752 | PPP3CA | rs2063058 | 4 | 102296004 | C | T |
| 1753 | PPP3CA | rs2695213 | 4 | 102298698 | G | A |
| 1754 | PPP3CA | rs2695219 | 4 | 102302914 | A | C |
| 1755 | PPP3CA | rs4478191 | 4 | 102303777 | C | T |
| 1756 | PPP3CA | rs4699110 | 4 | 102313475 | T | G |
| 1757 | PPP3CA | rs2850982 | 4 | 102319393 | T | C |
| 1758 | PPP3CA | rs1848116 | 4 | 102322962 | T | C |
| 1759 | PPP3CA | rs2659506 | 4 | 102328086 | C | T |
| 1760 | PPP3CA | rs2659509 | 4 | 102332046 | C | A |
| 1761 | PPP3CA | rs2659511 | 4 | 102332103 | G | A |
| 1762 | PPP3CA | rs3811063 | 4 | 102339312 | G | A |
| 1763 | PPP3CA | rs2850992 | 4 | 102341777 | T | C |
| 1764 | PPP3CA | rs2659533 | 4 | 102348552 | C | T |
| 1765 | PPP3CA | rs1157038 | 4 | 102359279 | C | A |
| 1766 | PPP3CA | rs713455 | 4 | 102366667 | C | G |
| 1767 | PPP3CA | rs978373 | 4 | 102373366 | C | T |
| 1768 | PPP3CA | rs2850950 | 4 | 102377472 | G | A |
| 1769 | PPP3CA | rs2850965 | 4 | 102397985 | T | G |
| 1770 | PPP3CA | rs3804420 | 4 | 102400605 | G | A |
| 1771 | PPP3CA | rs2850971 | 4 | 102403648 | C | T |
| 1772 | PPP3CA | rs2583401 | 4 | 102423551 | G | A |
| 1773 | PPP3CA | rs1441433 | 4 | 102428382 | A | C |
| 1774 | PPP3CA | rs6852347 | 4 | 102436589 | T | C |
| 1775 | PPP3CA | rs1348161 | 4 | 102471718 | G | A |
| 1776 | PPP3CA | rs10516469 | 4 | 102480275 | G | A |
| 1777 | PPP3CA | rs6810812 | 4 | 102483984 | G | A |
| 1778 | PPP3CB | rs12644 | 10 | 74867056 | G | A |
| 1779 | PPP3CB | rs11000666 | 10 | 74902999 | G | T |
| 1780 | PPP3CB | rs11000671 | 10 | 74909982 | C | T |
| 1781 | PPP3CB | rs7099640 | 10 | 74926429 | C | A |
| 1782 | PPP3CB | rs7099917 | 10 | 74926606 | C | A |
| 1783 | PPP3CC | rs7837713 | 8 | 22375142 | T | C |
| 1784 | PPP3CC | rs7827093 | 8 | 22390554 | T | C |
| 1785 | PPP3CC | rs2443502 | 8 | 22396443 | G | A |
| 1786 | PPP3CC | rs2461493 | 8 | 22409513 | A | G |
| 1787 | PPP3CC | rs2469749 | 8 | 22413542 | C | T |
| 1788 | PPP3CC | rs2469760 | 8 | 22421889 | A | G |
| 1789 | PPP3CC | rs2461490 | 8 | 22426059 | C | G |
| 1790 | PPP3CC | rs2461489 | 8 | 22426197 | C | T |
| 1791 | PPP3CC | rs2100291 | 8 | 22430495 | G | A |
| 1792 | PPP3CC | rs2461483 | 8 | 22442423 | C | T |
| 1793 | PPP3CC | rs2449340 | 8 | 22445844 | T | G |
| 1794 | PPP3R1 | rs1868402 | 2 | 68262540 | T | C |
| 1795 | PPP3R1 | rs6757991 | 2 | 68264163 | G | T |
| 1796 | PPP3R1 | rs6546358 | 2 | 68264917 | A | G |
| 1797 | PPP3R1 | rs6758449 | 2 | 68290611 | G | A |
| 1798 | PPP3R1 | rs6754495 | 2 | 68296768 | G | A |
| 1799 | PPP3R2 | rs3739723 | 9 | 103394170 | T | A |
| 1800 | PPP3R2 | rs1407877 | 9 | 103395275 | G | A |
| 1801 | PPP3R2 | rs3739724 | 9 | 103396259 | T | C |
| 1802 | PPP3R2 | rs6479056 | 9 | 103399081 | A | G |
| 1803 | PTK2 | rs7460 | 8 | 141738041 | A | T |
| 1804 | PTK2 | rs13273096 | 8 | 141745112 | T | C |
| 1805 | PTK2 | rs11166992 | 8 | 141746651 | T | C |
| 1806 | PTK2 | rs6998337 | 8 | 141754822 | A | C |
| 1807 | PTK2 | rs7820179 | 8 | 141770657 | T | C |
| 1808 | PTK2 | rs11785444 | 8 | 141774177 | G | A |
| 1809 | PTK2 | rs4961287 | 8 | 141775501 | G | A |
| 1810 | PTK2 | rs4596627 | 8 | 141777575 | C | T |
| 1811 | PTK2 | rs10109684 | 8 | 141782955 | A | G |
| 1812 | PTK2 | rs10100025 | 8 | 141785465 | G | A |
| 1813 | PTK2 | rs11166995 | 8 | 141788432 | G | A |
| 1814 | PTK2 | rs4246122 | 8 | 141794981 | T | C |
| 1815 | PTK2 | rs13280245 | 8 | 141796491 | G | T |
| 1816 | PTK2 | rs7839832 | 8 | 141797655 | C | T |
| 1817 | PTK2 | rs13257090 | 8 | 141798644 | C | T |
| 1818 | PTK2 | rs11997161 | 8 | 141807768 | C | T |
| 1819 | PTK2 | rs6994744 | 8 | 141810049 | A | C |
| 1820 | PTK2 | rs4961289 | 8 | 141817446 | G | T |
| 1821 | PTK2 | rs3923115 | 8 | 141823632 | C | T |
| 1822 | PTK2 | rs6993266 | 8 | 141831840 | A | G |
| 1823 | PTK2 | rs7840381 | 8 | 141843463 | A | C |
| 1824 | PTK2 | rs4378011 | 8 | 141843757 | A | G |
| 1825 | PTK2 | rs4246123 | 8 | 141844349 | C | A |
| 1826 | PTK2 | rs4961233 | 8 | 141878129 | C | G |
| 1827 | PTK2 | rs10090774 | 8 | 141895920 | C | T |
| 1828 | PTK2 | rs4434666 | 8 | 141909349 | A | G |
| 1829 | PTK2 | rs4246128 | 8 | 141909920 | G | A |
| 1830 | PTK2 | rs13438888 | 8 | 141911123 | T | A |
| 1831 | PTK2 | rs7831543 | 8 | 141916430 | T | C |
| 1832 | PTK2 | rs7815191 | 8 | 141924837 | C | G |
| 1833 | PTK2 | rs6999145 | 8 | 141941542 | G | — |
| 1834 | PTK2 | rs13271554 | 8 | 141942869 | G | A |
| 1835 | PTK2 | rs7008498 | 8 | 141946209 | G | A |
| 1836 | PTK2 | rs13261338 | 8 | 141961002 | G | A |
| 1837 | PTK2 | rs1868280 | 8 | 141965435 | T | C |
| 1838 | PTK2 | rs11991796 | 8 | 141967699 | C | T |
| 1839 | PTK2 | rs4961237 | 8 | 141971674 | G | A |
| 1840 | PTK2 | rs12544802 | 8 | 142012927 | G | A |
| 1841 | PTK2 | rs1375062 | 8 | 142034962 | A | C |
| 1842 | PTK2 | rs4440675 | 8 | 142044638 | C | T |
| 1843 | PTK2 | rs11781807 | 8 | 142063574 | G | T |
| 1844 | PTK2 | rs11167016 | 8 | 142064361 | G | A |
| 1845 | RAC1 | rs7456834 | 7 | 6383112 | G | C |
| 1846 | RAC1 | rs2347338 | 7 | 6384802 | C | T |
| 1847 | RAC1 | rs10951982 | 7 | 6389080 | G | A |
| 1848 | RAC1 | rs6967221 | 7 | 6397615 | G | A |
| 1849 | RAC1 | rs836480 | 7 | 6397928 | C | A |
| 1850 | RAC1 | rs836547 | 7 | 6405442 | G | T |
| 1851 | RAC1 | rs2303364 | 7 | 6408212 | T | C |
| 1852 | RAC2 | rs6572 | 22 | 35951390 | C | G |
| 1853 | RAC2 | rs9610680 | 22 | 35951896 | G | A |
| 1854 | RAC2 | rs739041 | 22 | 35954944 | A | G |
| 1855 | RAC2 | rs9610683 | 22 | 35957191 | G | C |
| 1856 | RAC2 | rs1476002 | 22 | 35960733 | C | T |
| 1857 | RAC2 | rs12170612 | 22 | 35962125 | G | T |
| 1858 | RAC2 | rs2284038 | 22 | 35965000 | A | G |
| 1859 | RAC2 | rs6000622 | 22 | 35967552 | A | — |
| 1860 | RAC2 | rs2239774 | 22 | 35967598 | G | C |
| 1861 | RAC2 | rs2239775 | 22 | 35967786 | C | A |
| 1862 | RAC2 | rs2213430 | 22 | 35968905 | C | T |
| 1863 | RASA1 | rs3734000 | 5 | 86600750 | T | A |
| 1864 | RASA1 | rs1567348 | 5 | 86621629 | G | A |
| 1865 | RASA1 | rs6893772 | 5 | 86632986 | G | A |
| 1866 | RASA1 | rs13362486 | 5 | 86639580 | C | A |
| 1867 | RASA1 | rs3752862 | 5 | 86669728 | G | A |
| 1868 | RASA1 | rs10942502 | 5 | 86671697 | A | C |
| 1869 | RASA1 | rs4421140 | 5 | 86692719 | T | C |
| 1870 | RASA1 | rs10067098 | 5 | 86706725 | T | C |
| 1871 | RASA1 | rs3827607 | 5 | 86712975 | G | A |
| 1872 | RGS3 | rs6478011 | 9 | 115284802 | T | C |
| 1873 | RGS3 | rs6478012 | 9 | 115286705 | T | C |
| 1874 | RGS3 | rs7860673 | 9 | 115298375 | G | A |
| 1875 | RGS3 | rs3761820 | 9 | 115306524 | T | C |
| 1876 | RGS3 | rs10481676 | 9 | 115312501 | T | C |
| 1877 | RGS3 | rs7045347 | 9 | 115314020 | G | C |
| 1878 | RGS3 | rs12342508 | 9 | 115314400 | A | G |
| 1879 | RGS3 | rs7852118 | 9 | 115328084 | T | C |
| 1880 | RGS3 | rs7851718 | 9 | 115343985 | G | A |
| 1881 | RGS3 | rs4979257 | 9 | 115374597 | A | G |
| 1882 | RGS3 | rs3810927 | 9 | 115386107 | C | T |
| 1883 | RGS3 | rs10739393 | 9 | 115393182 | T | C |
| 1884 | RGS3 | rs4979261 | 9 | 115395235 | A | G |
| 1885 | RGS3 | rs12341266 | 9 | 115396336 | G | A |
| 1886 | RHOA | rs3448 | 3 | 49371754 | C | T |
| 1887 | RHOA | rs7621003 | 3 | 49386407 | T | C |
| 1888 | RHOA | rs940045 | 3 | 49424641 | T | C |
| 1889 | RHOD | rs7112925 | 11 | 66582735 | C | T |
| 1890 | RHOD | rs2282502 | 11 | 66590827 | C | T |
| 1891 | RND1 | rs12828980 | 12 | 47538767 | A | — |
| 1892 | RND1 | rs1565933 | 12 | 47542064 | G | C |
| 1893 | RND1 | rs3782360 | 12 | 47543058 | G | A |
| 1894 | ROBO1 | rs1444480 | 3 | 78731221 | A | G |
| 1895 | ROBO1 | rs6786778 | 3 | 78733249 | A | G |
| 1896 | ROBO1 | rs723766 | 3 | 78740463 | G | A |
| 1897 | ROBO1 | rs723765 | 3 | 78740687 | G | A |
| 1898 | ROBO1 | rs1027828 | 3 | 78743374 | C | T |
| 1899 | ROBO1 | rs3773195 | 3 | 78745963 | G | A |
| 1900 | ROBO1 | rs1025946 | 3 | 78747341 | T | C |
| 1901 | ROBO1 | rs9839790 | 3 | 78749454 | C | T |
| 1902 | ROBO1 | rs3773201 | 3 | 78751142 | C | T |
| 1903 | ROBO1 | rs17016413 | 3 | 78753343 | C | T |
| 1904 | ROBO1 | rs7625555 | 3 | 78759094 | T | C |
| 1905 | ROBO1 | rs7614084 | 3 | 78759111 | G | A |
| 1906 | ROBO1 | rs6809232 | 3 | 78774965 | A | G |
| 1907 | ROBO1 | rs1995693 | 3 | 78777619 | C | T |
| 1908 | ROBO1 | rs1032966 | 3 | 78781077 | G | A |
| 1909 | ROBO1 | rs6795556 | 3 | 78783590 | G | T |
| 1910 | ROBO1 | rs998146 | 3 | 78787331 | G | A |
| 1911 | ROBO1 | rs7613490 | 3 | 78794473 | G | A |
| 1912 | ROBO1 | rs2271151 | 3 | 78800032 | G | A |
| 1913 | ROBO1 | rs2255164 | 3 | 78800197 | T | C |
| 1914 | ROBO1 | rs2043661 | 3 | 78816410 | A | G |
| 1915 | ROBO1 | rs3773228 | 3 | 78820081 | G | — |
| 1916 | ROBO1 | rs9880072 | 3 | 78836047 | C | T |
| 1917 | ROBO1 | rs767182 | 3 | 78837320 | C | T |
| 1918 | ROBO1 | rs984138 | 3 | 78839492 | A | G |
| 1919 | ROBO1 | rs2118067 | 3 | 78847345 | T | C |
| 1920 | ROBO1 | rs6787349 | 3 | 78851631 | C | T |
| 1921 | ROBO1 | rs3773234 | 3 | 78860200 | A | G |
| 1922 | ROBO1 | rs3773236 | 3 | 78861590 | G | T |
| 1923 | ROBO1 | rs11927671 | 3 | 78870558 | G | T |
| 1924 | ROBO1 | rs2608021 | 3 | 78872051 | G | A |
| 1925 | ROBO1 | rs2304503 | 3 | 78878767 | A | C |
| 1926 | ROBO1 | rs1865862 | 3 | 78886539 | C | A |
| 1927 | ROBO1 | rs333472 | 3 | 78907601 | T | C |
| 1928 | ROBO1 | rs333473 | 3 | 78907737 | A | — |
| 1929 | ROBO1 | rs7650002 | 3 | 78926963 | G | A |
| 1930 | ROBO1 | rs9309819 | 3 | 78930721 | C | T |
| 1931 | ROBO1 | rs11127635 | 3 | 78940491 | A | G |
| 1932 | ROBO1 | rs1355983 | 3 | 78945367 | T | G |
| 1933 | ROBO1 | rs7629522 | 3 | 78945847 | C | T |
| 1934 | ROBO1 | rs162870 | 3 | 78959906 | A | C |
| 1935 | ROBO1 | rs162871 | 3 | 78960198 | C | A |
| 1936 | ROBO1 | rs7617433 | 3 | 78966168 | G | A |
| 1937 | ROBO1 | rs1843786 | 3 | 78967413 | A | G |
| 1938 | ROBO1 | rs331158 | 3 | 78975041 | C | A |
| 1939 | ROBO1 | rs1480689 | 3 | 78983701 | C | A |
| 1940 | ROBO1 | rs1383407 | 3 | 78990860 | T | C |
| 1941 | ROBO1 | rs1031932 | 3 | 78995892 | A | C |
| 1942 | ROBO1 | rs331149 | 3 | 78997894 | A | G |
| 1943 | ROBO1 | rs331147 | 3 | 78999701 | A | C |
| 1944 | ROBO1 | rs331146 | 3 | 78999972 | A | G |
| 1945 | ROBO1 | rs11127636 | 3 | 79005541 | A | C |
| 1946 | ROBO1 | rs331199 | 3 | 79033571 | T | C |
| 1947 | ROBO1 | rs498805 | 3 | 79051530 | T | C |
| 1948 | ROBO1 | rs9839572 | 3 | 79066612 | A | C |
| 1949 | ROBO1 | rs328049 | 3 | 79068494 | C | T |
| 1950 | ROBO1 | rs10511126 | 3 | 79073205 | C | G |
| 1951 | ROBO1 | rs9832632 | 3 | 79098153 | C | T |
| 1952 | ROBO1 | rs1009682 | 3 | 79104085 | G | A |
| 1953 | ROBO1 | rs4680919 | 3 | 79108258 | T | C |
| 1954 | ROBO1 | rs1447833 | 3 | 79122095 | A | G |
| 1955 | ROBO1 | rs1374883 | 3 | 79146521 | T | C |
| 1956 | ROBO1 | rs6766522 | 3 | 79160576 | G | A |
| 1957 | ROBO1 | rs6548612 | 3 | 79206159 | C | T |
| 1958 | ROBO1 | rs1455824 | 3 | 79219919 | G | A |
| 1959 | ROBO1 | rs9866364 | 3 | 79240043 | T | C |
| 1960 | ROBO1 | rs1455833 | 3 | 79258556 | G | A |
| 1961 | ROBO1 | rs6786179 | 3 | 79259719 | G | A |
| 1962 | ROBO1 | rs7349498 | 3 | 79268773 | G | A |
| 1963 | ROBO1 | rs7631406 | 3 | 79273279 | C | T |
| 1964 | ROBO1 | rs1378633 | 3 | 79278481 | G | A |
| 1965 | ROBO1 | rs12486635 | 3 | 79280399 | A | G |
| 1966 | ROBO1 | rs9823652 | 3 | 79304260 | G | A |
| 1967 | ROBO1 | rs2077753 | 3 | 79347300 | T | C |
| 1968 | ROBO1 | rs11924816 | 3 | 79369490 | C | T |
| 1969 | ROBO1 | rs9880736 | 3 | 79401677 | A | C |
| 1970 | ROBO1 | rs12629261 | 3 | 79402666 | C | T |
| 1971 | ROBO1 | rs1489850 | 3 | 79411848 | T | C |
| 1972 | ROBO1 | rs7618349 | 3 | 79422279 | T | C |
| 1973 | ROBO1 | rs9832396 | 3 | 79436876 | G | A |
| 1974 | ROBO1 | rs6769328 | 3 | 79455525 | A | G |
| 1975 | ROBO1 | rs4362686 | 3 | 79476046 | G | A |
| 1976 | ROBO1 | rs13073607 | 3 | 79482169 | C | T |
| 1977 | ROBO1 | rs6548616 | 3 | 79482264 | T | C |
| 1978 | ROBO1 | rs9842630 | 3 | 79489208 | C | T |
| 1979 | ROBO1 | rs6802848 | 3 | 79493703 | G | A |
| 1980 | ROBO1 | rs6784911 | 3 | 79494881 | A | G |
| 1981 | ROBO1 | rs12633213 | 3 | 79504371 | T | C |
| 1982 | ROBO1 | rs4680960 | 3 | 79532255 | C | T |
| 1983 | ROBO1 | rs7653197 | 3 | 79553473 | C | A |
| 1984 | ROBO1 | rs4535189 | 3 | 79572660 | C | T |
| 1985 | ROBO1 | rs12635896 | 3 | 79613437 | T | C |
| 1986 | ROBO1 | rs4564923 | 3 | 79615784 | C | T |
| 1987 | ROBO1 | rs4264688 | 3 | 79629037 | G | A |
| 1988 | ROBO1 | rs6548621 | 3 | 79633062 | G | A |
| 1989 | ROBO1 | rs7622444 | 3 | 79640616 | A | G |
| 1990 | ROBO1 | rs7637338 | 3 | 79643293 | G | A |
| 1991 | ROBO1 | rs7614913 | 3 | 79650318 | T | C |
| 1992 | ROBO1 | rs7651819 | 3 | 79653121 | A | C |
| 1993 | ROBO1 | rs4527338 | 3 | 79671721 | G | A |
| 1994 | ROBO1 | rs7623728 | 3 | 79679724 | C | T |
| 1995 | ROBO1 | rs11924965 | 3 | 79693314 | C | T |
| 1996 | ROBO1 | rs9863111 | 3 | 79711783 | A | G |
| 1997 | ROBO1 | rs9872108 | 3 | 79716755 | G | T |
| 1998 | ROBO1 | rs12714483 | 3 | 79720107 | T | G |
| 1999 | ROBO1 | rs9855098 | 3 | 79722264 | C | A |
| 2000 | ROBO2 | rs3923745 | 3 | 77172084 | A | G |
| 2001 | ROBO2 | rs6784307 | 3 | 77176728 | C | A |
| 2002 | ROBO2 | rs3934881 | 3 | 77177488 | T | C |
| 2003 | ROBO2 | rs6779154 | 3 | 77187228 | T | C |
| 2004 | ROBO2 | rs7640155 | 3 | 77197617 | C | T |
| 2005 | ROBO2 | rs7628843 | 3 | 77216562 | A | G |
| 2006 | ROBO2 | rs9816491 | 3 | 77220967 | A | G |
| 2007 | ROBO2 | rs9873691 | 3 | 77229728 | C | A |
| 2008 | ROBO2 | rs9874095 | 3 | 77229801 | G | A |
| 2009 | ROBO2 | rs4624600 | 3 | 77234476 | A | G |
| 2010 | ROBO2 | rs9859970 | 3 | 77237122 | T | C |
| 2011 | ROBO2 | rs13322517 | 3 | 77238513 | A | G |
| 2012 | ROBO2 | rs13061481 | 3 | 77240876 | T | C |
| 2013 | ROBO2 | rs12185957 | 3 | 77247985 | A | C |
| 2014 | ROBO2 | rs6796664 | 3 | 77250164 | A | G |
| 2015 | ROBO2 | rs4077419 | 3 | 77270730 | A | C |
| 2016 | ROBO2 | rs9682089 | 3 | 77273493 | G | A |
| 2017 | ROBO2 | rs7615545 | 3 | 77278860 | G | A |
| 2018 | ROBO2 | rs7432300 | 3 | 77289776 | T | G |
| 2019 | ROBO2 | rs7428284 | 3 | 77301697 | G | T |
| 2020 | ROBO2 | rs9852548 | 3 | 77320759 | G | A |
| 2021 | ROBO2 | rs11709672 | 3 | 77326174 | T | C |
| 2022 | ROBO2 | rs9822245 | 3 | 77330939 | T | G |
| 2023 | ROBO2 | rs1110226 | 3 | 77332386 | A | G |
| 2024 | ROBO2 | rs9812479 | 3 | 77332426 | G | A |
| 2025 | ROBO2 | rs4684028 | 3 | 77355344 | A | G |
| 2026 | ROBO2 | rs7432274 | 3 | 77356600 | A | G |
| 2027 | ROBO2 | rs12487684 | 3 | 77364366 | A | G |
| 2028 | ROBO2 | rs17769969 | 3 | 77366336 | G | A |
| 2029 | ROBO2 | rs6772736 | 3 | 77368733 | G | A |
| 2030 | ROBO2 | rs6768395 | 3 | 77370794 | T | C |
| 2031 | ROBO2 | rs6764328 | 3 | 77378350 | C | T |
| 2032 | ROBO2 | rs4072806 | 3 | 77382123 | C | A |
| 2033 | ROBO2 | rs7430171 | 3 | 77391012 | C | T |
| 2034 | ROBO2 | rs1372423 | 3 | 77393053 | A | G |
| 2035 | ROBO2 | rs7640201 | 3 | 77394263 | T | C |
| 2036 | ROBO2 | rs1470203 | 3 | 77398107 | G | A |
| 2037 | ROBO2 | rs9882239 | 3 | 77405023 | T | C |
| 2038 | ROBO2 | rs9309764 | 3 | 77408430 | G | A |
| 2039 | ROBO2 | rs1992449 | 3 | 77411693 | A | G |
| 2040 | ROBO2 | rs10511057 | 3 | 77412081 | T | C |
| 2041 | ROBO2 | rs1441956 | 3 | 77415339 | G | A |
| 2042 | ROBO2 | rs2875436 | 3 | 77418845 | G | A |
| 2043 | ROBO2 | rs998037 | 3 | 77429652 | C | T |
| 2044 | ROBO2 | rs1899373 | 3 | 77436099 | G | A |
| 2045 | ROBO2 | rs10511054 | 3 | 77442048 | C | T |
| 2046 | ROBO2 | rs1372427 | 3 | 77451047 | A | G |
| 2047 | ROBO2 | rs1978940 | 3 | 77452600 | C | A |
| 2048 | ROBO2 | rs2028510 | 3 | 77460222 | G | T |
| 2049 | ROBO2 | rs2028512 | 3 | 77466145 | T | C |
| 2050 | ROBO2 | rs9868442 | 3 | 77466639 | G | A |
| 2051 | ROBO2 | rs10511055 | 3 | 77469824 | A | G |
| 2052 | ROBO2 | rs6548499 | 3 | 77472055 | C | T |
| 2053 | ROBO2 | rs4449296 | 3 | 77477229 | G | A |
| 2054 | ROBO2 | rs4539916 | 3 | 77479333 | G | A |
| 2055 | ROBO2 | rs10865559 | 3 | 77489197 | C | T |
| 2056 | ROBO2 | rs7642312 | 3 | 77491879 | G | A |
| 2057 | ROBO2 | rs9869100 | 3 | 77494922 | A | G |
| 2058 | ROBO2 | rs9809640 | 3 | 77497751 | A | G |
| 2059 | ROBO2 | rs9872037 | 3 | 77507389 | A | G |
| 2060 | ROBO2 | rs6768736 | 3 | 77513319 | G | A |
| 2061 | ROBO2 | rs6548505 | 3 | 77516661 | A | G |
| 2062 | ROBO2 | rs9873887 | 3 | 77518312 | C | T |
| 2063 | ROBO2 | rs4684005 | 3 | 77520492 | T | G |
| 2064 | ROBO2 | rs9836971 | 3 | 77525970 | G | A |
| 2065 | ROBO2 | rs6548507 | 3 | 77527055 | G | A |
| 2066 | ROBO2 | rs9883718 | 3 | 77527121 | A | G |
| 2067 | ROBO2 | rs7432690 | 3 | 77533052 | C | T |
| 2068 | ROBO2 | rs12492221 | 3 | 77538614 | G | A |
| 2069 | ROBO2 | rs4327364 | 3 | 77544044 | G | T |
| 2070 | ROBO2 | rs9830337 | 3 | 77554151 | A | G |
| 2071 | ROBO2 | rs4459877 | 3 | 77556150 | G | A |
| 2072 | ROBO2 | rs11127595 | 3 | 77557820 | G | A |
| 2073 | ROBO2 | rs12634690 | 3 | 77568827 | C | T |
| 2074 | ROBO2 | rs6777631 | 3 | 77581313 | C | A |
| 2075 | ROBO2 | rs9840819 | 3 | 77586187 | G | A |
| 2076 | ROBO2 | rs7651943 | 3 | 77590213 | C | T |
| 2077 | ROBO2 | rs4684011 | 3 | 77600300 | C | T |
| 2078 | ROBO2 | rs7614439 | 3 | 77603995 | T | C |
| 2079 | ROBO2 | rs1345179 | 3 | 77607822 | C | T |
| 2080 | ROBO2 | rs12487172 | 3 | 77610216 | G | A |
| 2081 | ROBO2 | rs1470569 | 3 | 77614615 | T | C |
| 2082 | ROBO2 | rs6789690 | 3 | 77621228 | A | T |
| 2083 | ROBO2 | rs13064369 | 3 | 77621907 | T | C |
| 2084 | ROBO2 | rs6779560 | 3 | 77628953 | A | G |
| 2085 | ROBO2 | rs13077150 | 3 | 77641074 | G | A |
| 2086 | ROBO2 | rs935526 | 3 | 77642706 | T | C |
| 2087 | ROBO2 | rs1447846 | 3 | 77652510 | G | A |
| 2088 | ROBO2 | rs812887 | 3 | 77658851 | C | T |
| 2089 | ROBO2 | rs775762 | 3 | 77671178 | T | C |
| 2090 | ROBO2 | rs775764 | 3 | 77672606 | C | T |
| 2091 | ROBO2 | rs1666130 | 3 | 77681632 | A | G |
| 2092 | ROBO2 | rs1523759 | 3 | 77686931 | T | C |
| 2093 | ROBO2 | rs10514734 | 3 | 77687379 | C | T |
| 2094 | ROBO2 | rs1403848 | 3 | 77692344 | A | C |
| 2095 | ROBO2 | rs1721188 | 3 | 77692564 | A | G |
| 2096 | ROBO2 | rs775713 | 3 | 77699908 | C | G |
| 2097 | ROBO2 | rs1107432 | 3 | 77715083 | C | T |
| 2098 | ROBO2 | rs775733 | 3 | 77720356 | C | T |
| 2099 | ROBO2 | rs1403847 | 3 | 77730422 | G | C |
| 2100 | ROBO2 | rs1721175 | 3 | 77733383 | T | C |
| 2101 | ROBO2 | rs9852724 | 3 | 77734674 | C | T |
| 2102 | ROBO2 | rs1523768 | 3 | 77749733 | T | C |
| 2103 | ROBO2 | rs9848106 | 3 | 77751035 | A | G |
| 2104 | ROBO2 | rs4684016 | 3 | 77756597 | A | G |
| 2105 | ROBO2 | rs876675 | 3 | 77758327 | A | G |
| 2106 | ROBO2 | rs10779982 | 3 | 77760995 | G | A |
| 2107 | ROBO2 | rs3821735 | 3 | 77766911 | G | A |
| 2108 | ROBO2 | rs9845024 | 3 | 77775532 | A | C |
| 2109 | ROBO2 | rs1839795 | 3 | 77775814 | C | T |
| 2110 | ROBO2 | rs1031377 | 3 | 77779111 | T | C |
| 2111 | ROBO3 | rs5017048 | 11 | 124239680 | G | T |
| 2112 | ROBO3 | rs7951801 | 11 | 124239937 | G | A |
| 2113 | ROBO3 | rs7102701 | 11 | 124242491 | T | C |
| 2114 | ROBO3 | rs4936957 | 11 | 124245197 | C | T |
| 2115 | ROBO3 | rs3862618 | 11 | 124247574 | C | T |
| 2116 | ROBO3 | rs4606490 | 11 | 124247940 | T | C |
| 2117 | ROBO3 | rs10790713 | 11 | 124252542 | A | G |
| 2118 | ROBO3 | rs3802905 | 11 | 124254793 | C | G |
| 2119 | ROCK1 | rs7244299 | 18 | 16810502 | C | T |
| 2120 | ROCK1 | rs8089751 | 18 | 16821951 | T | — |
| 2121 | ROCK1 | rs8085654 | 18 | 16824232 | G | A |
| 2122 | ROCK1 | rs11877273 | 18 | 16848679 | C | T |
| 2123 | ROCK1 | rs288980 | 18 | 16863577 | C | T |
| 2124 | ROCK1 | rs7239317 | 18 | 16864064 | C | T |
| 2125 | ROCK1 | rs7240577 | 18 | 16880539 | C | — |
| 2126 | ROCK1 | rs7238386 | 18 | 16881027 | T | C |
| 2127 | ROCK1 | rs2127958 | 18 | 16907607 | T | C |
| 2128 | ROCK1 | rs1481280 | 18 | 16909448 | C | A |
| 2129 | ROCK1 | rs1006881 | 18 | 16935290 | G | A |
| 2130 | ROCK1 | rs11874761 | 18 | 16945763 | G | A |
| 2131 | ROCK2 | rs6753921 | 2 | 11244205 | G | A |
| 2132 | ROCK2 | rs2290156 | 2 | 11250658 | G | C |
| 2133 | ROCK2 | rs726843 | 2 | 11273640 | C | T |
| 2134 | ROCK2 | rs1515219 | 2 | 11286528 | C | T |
| 2135 | ROCK2 | rs1868585 | 2 | 11300279 | C | T |
| 2136 | ROCK2 | rs6716817 | 2 | 11303284 | G | A |
| 2137 | ROCK2 | rs6730673 | 2 | 11311122 | G | T |
| 2138 | ROCK2 | rs10192593 | 2 | 11318862 | A | G |
| 2139 | ROCK2 | rs7563468 | 2 | 11348725 | A | G |
| 2140 | ROCK2 | rs10203916 | 2 | 11369434 | T | C |
| 2141 | ROCK2 | rs12622447 | 2 | 11392895 | G | A |
| 2142 | ROCK2 | rs12692437 | 2 | 11398011 | T | C |
| 2143 | RRAS | rs1368464 | 19 | 54831879 | A | G |
| 2144 | RRAS | rs1865077 | 19 | 54831903 | T | C |
| 2145 | RRAS2 | rs2303972 | 11 | 14259857 | A | T |
| 2146 | RRAS2 | rs11023177 | 11 | 14263155 | G | A |
| 2147 | RRAS2 | rs3900595 | 11 | 14269904 | A | C |
| 2148 | RRAS2 | rs12421958 | 11 | 14274838 | C | A |
| 2149 | RRAS2 | rs2970334 | 11 | 14285930 | G | A |
| 2150 | RRAS2 | rs10832242 | 11 | 14297079 | T | C |
| 2151 | RRAS2 | rs2915409 | 11 | 14312307 | T | C |
| 2152 | RRAS2 | rs2915400 | 11 | 14321526 | C | T |
| 2153 | RRAS2 | rs11023194 | 11 | 14326344 | T | C |
| 2154 | SEMA3A | rs10239654 | 7 | 83428166 | G | A |
| 2155 | SEMA3A | rs797821 | 7 | 83428787 | G | A |
| 2156 | SEMA3A | rs797810 | 7 | 83434618 | G | A |
| 2157 | SEMA3A | rs740948 | 7 | 83437270 | C | T |
| 2158 | SEMA3A | rs701320 | 7 | 83444453 | T | C |
| 2159 | SEMA3A | rs2535785 | 7 | 83451992 | G | C |
| 2160 | SEMA3A | rs740947 | 7 | 83454463 | A | G |
| 2161 | SEMA3A | rs3801599 | 7 | 83470484 | C | T |
| 2162 | SEMA3A | rs7804122 | 7 | 83472648 | A | G |
| 2163 | SEMA3A | rs4732529 | 7 | 83473521 | C | T |
| 2164 | SEMA3A | rs1404480 | 7 | 83476774 | T | C |
| 2165 | SEMA3A | rs10499878 | 7 | 83477622 | A | G |
| 2166 | SEMA3A | rs17158505 | 7 | 83480490 | G | T |
| 2167 | SEMA3A | rs1534075 | 7 | 83482920 | T | G |
| 2168 | SEMA3A | rs6949238 | 7 | 83484751 | T | C |
| 2169 | SEMA3A | rs1533997 | 7 | 83489736 | A | T |
| 2170 | SEMA3A | rs6976211 | 7 | 83489789 | C | T |
| 2171 | SEMA3A | rs1404473 | 7 | 83505108 | T | C |
| 2172 | SEMA3A | rs2372025 | 7 | 83512258 | T | C |
| 2173 | SEMA3A | rs4355720 | 7 | 83516887 | A | G |
| 2174 | SEMA3A | rs2527040 | 7 | 83524083 | A | G |
| 2175 | SEMA3A | rs2527032 | 7 | 83525557 | C | T |
| 2176 | SEMA3A | rs2189849 | 7 | 83529506 | C | A |
| 2177 | SEMA3A | rs2527031 | 7 | 83536112 | T | C |
| 2178 | SEMA3A | rs10488266 | 7 | 83539612 | T | C |
| 2179 | SEMA3A | rs2691694 | 7 | 83540411 | G | A |
| 2180 | SEMA3A | rs2691699 | 7 | 83554955 | A | G |
| 2181 | SEMA3A | rs3801615 | 7 | 83558709 | C | T |
| 2182 | SEMA3A | rs917089 | 7 | 83568384 | A | C |
| 2183 | SEMA3A | rs917090 | 7 | 83571952 | T | C |
| 2184 | SEMA3A | rs3801631 | 7 | 83572924 | G | A |
| 2185 | SEMA3A | rs764116 | 7 | 83576416 | T | C |
| 2186 | SEMA3A | rs1990044 | 7 | 83577697 | C | T |
| 2187 | SEMA3A | rs3801650 | 7 | 83580879 | T | A |
| 2188 | SEMA3A | rs929334 | 7 | 83582308 | C | T |
| 2189 | SEMA3A | rs2372033 | 7 | 83584248 | T | C |
| 2190 | SEMA3A | rs7793598 | 7 | 83597043 | T | C |
| 2191 | SEMA3A | rs2067693 | 7 | 83597505 | A | G |
| 2192 | SEMA3A | rs10488270 | 7 | 83608283 | C | A |
| 2193 | SEMA3A | rs7781152 | 7 | 83620370 | C | T |
| 2194 | SEMA3A | rs17158686 | 7 | 83632691 | T | G |
| 2195 | SEMA3A | rs11974667 | 7 | 83634555 | C | A |
| 2196 | SEMA3A | rs12666837 | 7 | 83639292 | G | A |
| 2197 | SEMA3A | rs13224698 | 7 | 83647046 | A | C |
| 2198 | SEMA3A | rs12707628 | 7 | 83655830 | C | T |
| 2199 | SEMA3C | rs1527473 | 7 | 80211257 | C | A |
| 2200 | SEMA3C | rs12113860 | 7 | 80211663 | C | T |
| 2201 | SEMA3C | rs1949972 | 7 | 80212373 | C | T |
| 2202 | SEMA3C | rs1358340 | 7 | 80213121 | C | A |
| 2203 | SEMA3C | rs2886793 | 7 | 80216110 | C | T |
| 2204 | SEMA3C | rs3807094 | 7 | 80226071 | G | A |
| 2205 | SEMA3C | rs3807101 | 7 | 80231353 | C | T |
| 2206 | SEMA3C | rs12671126 | 7 | 80241120 | C | T |
| 2207 | SEMA3C | rs3778677 | 7 | 80249039 | C | T |
| 2208 | SEMA3C | rs17275986 | 7 | 80256624 | A | G |
| 2209 | SEMA3C | rs1544005 | 7 | 80262026 | G | A |
| 2210 | SEMA3C | rs12666387 | 7 | 80279412 | A | G |
| 2211 | SEMA3C | rs12668734 | 7 | 80282511 | T | C |
| 2212 | SEMA3C | rs12535655 | 7 | 80289750 | T | C |
| 2213 | SEMA3C | rs12670551 | 7 | 80291679 | A | G |
| 2214 | SEMA3C | rs12155128 | 7 | 80292870 | T | C |
| 2215 | SEMA3C | rs2040908 | 7 | 80297951 | A | C |
| 2216 | SEMA3C | rs929346 | 7 | 80300839 | C | T |
| 2217 | SEMA3C | rs17228184 | 7 | 80306736 | A | G |
| 2218 | SEMA3C | rs4731861 | 7 | 80309470 | C | T |
| 2219 | SEMA3C | rs1016840 | 7 | 80311981 | T | C |
| 2220 | SEMA3C | rs6956461 | 7 | 80357189 | T | C |
| 2221 | SEMA3C | rs7793555 | 7 | 80384680 | G | A |
| 2222 | SEMA3C | rs2057880 | 7 | 80387868 | C | A |
| 2223 | SEMA3D | rs2040957 | 7 | 84464323 | C | T |
| 2224 | SEMA3D | rs10261725 | 7 | 84465166 | G | A |
| 2225 | SEMA3D | rs7800072 | 7 | 84466924 | T | G |
| 2226 | SEMA3D | rs2286184 | 7 | 84468451 | C | T |
| 2227 | SEMA3D | rs10486845 | 7 | 84477067 | G | A |
| 2228 | SEMA3D | rs1468396 | 7 | 84481699 | T | C |
| 2229 | SEMA3D | rs7793649 | 7 | 84484404 | A | G |
| 2230 | SEMA3D | rs1016027 | 7 | 84490923 | A | C |
| 2231 | SEMA3D | rs2158727 | 7 | 84506380 | A | C |
| 2232 | SEMA3D | rs2040954 | 7 | 84507604 | T | C |
| 2233 | SEMA3D | rs2286190 | 7 | 84527928 | A | T |
| 2234 | SEMA3D | rs2240394 | 7 | 84530876 | T | C |
| 2235 | SEMA3D | rs6966472 | 7 | 84537322 | T | C |
| 2236 | SEMA3D | rs12673176 | 7 | 84538568 | G | T |
| 2237 | SEMA3D | rs6962019 | 7 | 84545860 | C | T |
| 2238 | SEMA3D | rs10260011 | 7 | 84547291 | C | T |
| 2239 | SEMA3D | rs12536409 | 7 | 84559062 | G | T |
| 2240 | SEMA3D | rs10486848 | 7 | 84575618 | G | A |
| 2241 | SEMA3D | rs1029563 | 7 | 84576430 | T | G |
| 2242 | SEMA3D | rs993312 | 7 | 84585779 | C | A |
| 2243 | SEMA3D | rs2190208 | 7 | 84589886 | T | C |
| 2244 | SEMA3E | rs2371542 | 7 | 82831743 | T | — |
| 2245 | SEMA3E | rs2371543 | 7 | 82831768 | T | — |
| 2246 | SEMA3E | rs6961363 | 7 | 82833008 | A | — |
| 2247 | SEMA3E | rs4473980 | 7 | 82834791 | T | — |
| 2248 | SEMA3E | rs3801479 | 7 | 82852931 | A | G |
| 2249 | SEMA3E | rs2709922 | 7 | 82863253 | G | A |
| 2250 | SEMA3E | rs7777731 | 7 | 82865280 | A | G |
| 2251 | SEMA3E | rs2255906 | 7 | 82867215 | C | A |
| 2252 | SEMA3E | rs2722974 | 7 | 82867373 | C | T |
| 2253 | SEMA3E | rs2722976 | 7 | 82870122 | C | T |
| 2254 | SEMA3E | rs17285647 | 7 | 82871576 | T | C |
| 2255 | SEMA3E | rs2245312 | 7 | 82874302 | C | G |
| 2256 | SEMA3E | rs2722983 | 7 | 82874829 | T | C |
| 2257 | SEMA3E | rs2722985 | 7 | 82875686 | A | C |
| 2258 | SEMA3E | rs2713154 | 7 | 82881203 | A | G |
| 2259 | SEMA3E | rs2722964 | 7 | 82896762 | C | T |
| 2260 | SEMA3E | rs2713174 | 7 | 82913892 | T | C |
| 2261 | SEMA3E | rs2723022 | 7 | 82917344 | A | G |
| 2262 | SEMA3E | rs2713178 | 7 | 82925128 | C | A |
| 2263 | SEMA3E | rs2722990 | 7 | 82939419 | T | G |
| 2264 | SEMA3E | rs4732512 | 7 | 82941990 | G | A |
| 2265 | SEMA3E | rs10270844 | 7 | 82946972 | C | T |
| 2266 | SEMA3E | rs2255614 | 7 | 82954321 | C | T |
| 2267 | SEMA3E | rs9640942 | 7 | 82956810 | G | T |
| 2268 | SEMA3E | rs2713192 | 7 | 82962945 | C | T |
| 2269 | SEMA3E | rs11982066 | 7 | 82972394 | T | C |
| 2270 | SEMA3E | rs2535372 | 7 | 82982047 | T | C |
| 2271 | SEMA3E | rs12530588 | 7 | 82986147 | G | A |
| 2272 | SEMA3E | rs1860610 | 7 | 82987499 | A | C |
| 2273 | SEMA3E | rs7779140 | 7 | 82987670 | G | A |
| 2274 | SEMA3E | rs2535368 | 7 | 82995142 | G | A |
| 2275 | SEMA3E | rs988578 | 7 | 82999149 | C | T |
| 2276 | SEMA3E | rs10255137 | 7 | 83008912 | A | G |
| 2277 | SEMA3E | rs1693381 | 7 | 83011867 | T | C |
| 2278 | SEMA3E | rs2058346 | 7 | 83012363 | G | A |
| 2279 | SEMA3E | rs1693389 | 7 | 83014197 | T | C |
| 2280 | SEMA3E | rs1639304 | 7 | 83017557 | A | G |
| 2281 | SEMA3E | rs2371784 | 7 | 83021658 | G | A |
| 2282 | SEMA3E | rs1468621 | 7 | 83025520 | A | G |
| 2283 | SEMA3E | rs215276 | 7 | 83031304 | T | C |
| 2284 | SEMA3E | rs1979768 | 7 | 83044508 | G | T |
| 2285 | SEMA3E | rs215311 | 7 | 83057202 | A | G |
| 2286 | SEMA3E | rs215314 | 7 | 83059943 | C | T |
| 2287 | SEMA3E | rs727549 | 7 | 83065909 | A | G |
| 2288 | SEMA3E | rs42002 | 7 | 83099020 | A | G |
| 2289 | SEMA3E | rs536299 | 7 | 83105918 | C | A |
| 2290 | SEMA3E | rs3757621 | 7 | 83107428 | G | A |
| 2291 | SEMA3E | rs302119 | 7 | 83113318 | G | A |
| 2292 | SEMA3E | rs302113 | 7 | 83118133 | G | T |
| 2293 | SEMA3F | rs2624842 | 3 | 50171421 | T | G |
| 2294 | SEMA3F | rs2072054 | 3 | 50171626 | T | C |
| 2295 | SEMA3F | rs2624839 | 3 | 50177234 | A | G |
| 2296 | SEMA3F | rs1005678 | 3 | 50185292 | G | C |
| 2297 | SEMA3F | rs12632110 | 3 | 50199228 | G | A |
| 2298 | SEMA3F | rs11711407 | 3 | 50200032 | G | A |
| 2299 | SEMA4A | rs3738582 | 1 | 154389853 | C | G |
| 2300 | SEMA4A | rs12128066 | 1 | 154393991 | T | C |
| 2301 | SEMA4A | rs510441 | 1 | 154396591 | A | G |
| 2302 | SEMA4A | rs1984508 | 1 | 154398240 | C | T |
| 2303 | SEMA4A | rs12401573 | 1 | 154412841 | T | C |
| 2304 | SEMA4A | rs7695 | 1 | 154413949 | T | C |
| 2305 | SEMA4A | rs3738581 | 1 | 154414181 | G | A |
| 2306 | SEMA4B | rs6496644 | 15 | 88547159 | C | T |
| 2307 | SEMA4B | rs1543116 | 15 | 88561994 | A | G |
| 2308 | SEMA4B | rs1543115 | 15 | 88562139 | C | T |
| 2309 | SEMA4B | rs4932305 | 15 | 88565222 | G | A |
| 2310 | SEMA4B | rs3751656 | 15 | 88572707 | C | T |
| 2311 | SEMA4B | rs3751655 | 15 | 88572753 | C | A |
| 2312 | SEMA4B | rs11547962 | 15 | 88573008 | G | A |
| 2313 | SEMA4B | rs1044246 | 15 | 88573203 | G | A |
| 2314 | SEMA4B | rs7167139 | 15 | 88573385 | G | C |
| 2315 | SEMA4B | rs8033436 | 15 | 88574145 | G | A |
| 2316 | SEMA4C | rs6576989 | 2 | 96888825 | T | C |
| 2317 | SEMA4D | rs9410474 | 9 | 91202539 | C | T |
| 2318 | SEMA4D | rs7045789 | 9 | 91206735 | G | A |
| 2319 | SEMA4D | rs11265799 | 9 | 91212290 | G | A |
| 2320 | SEMA4D | rs9410321 | 9 | 91220005 | T | C |
| 2321 | SEMA4D | rs4074459 | 9 | 91224757 | A | C |
| 2322 | SEMA4D | rs4132699 | 9 | 91226246 | A | C |
| 2323 | SEMA4D | rs10512197 | 9 | 91230294 | G | A |
| 2324 | SEMA4D | rs10797126 | 9 | 91233003 | G | A |
| 2325 | SEMA4D | rs4078338 | 9 | 91237252 | C | T |
| 2326 | SEMA4D | rs4073713 | 9 | 91242077 | G | A |
| 2327 | SEMA4D | rs9410486 | 9 | 91259490 | A | G |
| 2328 | SEMA4D | rs4360376 | 9 | 91264248 | A | C |
| 2329 | SEMA4D | rs9969727 | 9 | 91264653 | C | T |
| 2330 | SEMA4D | rs11265909 | 9 | 91267965 | T | C |
| 2331 | SEMA4D | rs10908927 | 9 | 91280760 | T | A |
| 2332 | SEMA4F | rs205648 | 2 | 74754002 | A | G |
| 2333 | SEMA4F | rs363608 | 2 | 74755109 | C | T |
| 2334 | SEMA4F | rs363611 | 2 | 74759707 | C | T |
| 2335 | SEMA4F | rs3025994 | 2 | 74761806 | C | T |
| 2336 | SEMA4G | rs4919510 | 10 | 102724767 | C | G |
| 2337 | SEMA4G | rs2295716 | 10 | 102734203 | A | G |
| 2338 | SEMA4G | rs722435 | 10 | 102735051 | G | T |
| 2339 | SEMA5A | rs786843 | 5 | 9093140 | C | G |
| 2340 | SEMA5A | rs2290735 | 5 | 9097410 | T | C |
| 2341 | SEMA5A | rs257098 | 5 | 9103409 | A | G |
| 2342 | SEMA5A | rs257095 | 5 | 9106944 | A | G |
| 2343 | SEMA5A | rs786847 | 5 | 9119463 | C | T |
| 2344 | SEMA5A | rs786846 | 5 | 9119801 | T | C |
| 2345 | SEMA5A | rs2892421 | 5 | 9129916 | A | G |
| 2346 | SEMA5A | rs1505062 | 5 | 9135480 | G | A |
| 2347 | SEMA5A | rs1013720 | 5 | 9140920 | T | C |
| 2348 | SEMA5A | rs253634 | 5 | 9144163 | G | A |
| 2349 | SEMA5A | rs2456237 | 5 | 9158282 | T | C |
| 2350 | SEMA5A | rs3777245 | 5 | 9163491 | A | G |
| 2351 | SEMA5A | rs3777247 | 5 | 9163735 | A | T |
| 2352 | SEMA5A | rs3777253 | 5 | 9174590 | G | T |
| 2353 | SEMA5A | rs6894626 | 5 | 9175925 | C | T |
| 2354 | SEMA5A | rs10513005 | 5 | 9183960 | A | G |
| 2355 | SEMA5A | rs1382918 | 5 | 9185189 | T | C |
| 2356 | SEMA5A | rs6883525 | 5 | 9189486 | A | C |
| 2357 | SEMA5A | rs7723025 | 5 | 9204236 | C | A |
| 2358 | SEMA5A | rs984425 | 5 | 9206378 | C | T |
| 2359 | SEMA5A | rs899576 | 5 | 9208419 | C | A |
| 2360 | SEMA5A | rs886509 | 5 | 9209011 | G | A |
| 2361 | SEMA5A | rs2526121 | 5 | 9211741 | C | T |
| 2362 | SEMA5A | rs40721 | 5 | 9221230 | C | T |
| 2363 | SEMA5A | rs40718 | 5 | 9226879 | T | C |
| 2364 | SEMA5A | rs3777281 | 5 | 9230931 | T | C |
| 2365 | SEMA5A | rs42353 | 5 | 9240034 | G | A |
| 2366 | SEMA5A | rs40701 | 5 | 9242566 | T | C |
| 2367 | SEMA5A | rs3797937 | 5 | 9244361 | G | A |
| 2368 | SEMA5A | rs40687 | 5 | 9248232 | T | C |
| 2369 | SEMA5A | rs1806086 | 5 | 9250547 | T | C |
| 2370 | SEMA5A | rs10513011 | 5 | 9252651 | G | A |
| 2371 | SEMA5A | rs1806112 | 5 | 9253136 | T | C |
| 2372 | SEMA5A | rs1806110 | 5 | 9253539 | C | T |
| 2373 | SEMA5A | rs1805971 | 5 | 9257141 | G | A |
| 2374 | SEMA5A | rs1805964 | 5 | 9262830 | C | A |
| 2375 | SEMA5A | rs3797954 | 5 | 9275044 | C | T |
| 2376 | SEMA5A | rs40669 | 5 | 9275810 | G | A |
| 2377 | SEMA5A | rs40666 | 5 | 9281170 | G | T |
| 2378 | SEMA5A | rs1805958 | 5 | 9287540 | C | T |
| 2379 | SEMA5A | rs187578 | 5 | 9287645 | G | A |
| 2380 | SEMA5A | rs10075485 | 5 | 9295883 | A | T |
| 2381 | SEMA5A | rs28181 | 5 | 9297525 | C | T |
| 2382 | SEMA5A | rs40713 | 5 | 9307908 | A | G |
| 2383 | SEMA5A | rs40708 | 5 | 9317681 | G | A |
| 2384 | SEMA5A | rs1805939 | 5 | 9319754 | T | C |
| 2385 | SEMA5A | rs1805918 | 5 | 9321736 | G | A |
| 2386 | SEMA5A | rs28802 | 5 | 9321808 | C | T |
| 2387 | SEMA5A | rs3777303 | 5 | 9333337 | A | G |
| 2388 | SEMA5A | rs6873909 | 5 | 9340072 | A | G |
| 2389 | SEMA5A | rs3777308 | 5 | 9349821 | T | C |
| 2390 | SEMA5A | rs3777324 | 5 | 9372586 | T | C |
| 2391 | SEMA5A | rs3777325 | 5 | 9372777 | C | G |
| 2392 | SEMA5A | rs3797980 | 5 | 9381315 | G | A |
| 2393 | SEMA5A | rs1205731 | 5 | 9388343 | T | C |
| 2394 | SEMA5A | rs1205729 | 5 | 9389544 | G | C |
| 2395 | SEMA5A | rs1205725 | 5 | 9396912 | C | T |
| 2396 | SEMA5A | rs1024488 | 5 | 9409923 | G | C |
| 2397 | SEMA5A | rs1024487 | 5 | 9410006 | G | A |
| 2398 | SEMA5A | rs3797996 | 5 | 9413640 | C | T |
| 2399 | SEMA5A | rs3798004 | 5 | 9423944 | C | T |
| 2400 | SEMA5A | rs194261 | 5 | 9429117 | A | G |
| 2401 | SEMA5A | rs40725 | 5 | 9438812 | G | A |
| 2402 | SEMA5A | rs42196 | 5 | 9439590 | G | T |
| 2403 | SEMA5A | rs390322 | 5 | 9443216 | G | A |
| 2404 | SEMA5A | rs10513007 | 5 | 9448452 | T | C |
| 2405 | SEMA5A | rs268489 | 5 | 9448672 | C | T |
| 2406 | SEMA5A | rs410741 | 5 | 9454605 | C | A |
| 2407 | SEMA5A | rs268487 | 5 | 9456973 | C | T |
| 2408 | SEMA5A | rs2463503 | 5 | 9462055 | G | A |
| 2409 | SEMA5A | rs6879538 | 5 | 9464248 | C | T |
| 2410 | SEMA5A | rs666617 | 5 | 9473373 | T | C |
| 2411 | SEMA5A | rs406898 | 5 | 9473644 | A | C |
| 2412 | SEMA5A | rs7724626 | 5 | 9477800 | G | T |
| 2413 | SEMA5A | rs3846587 | 5 | 9482199 | T | C |
| 2414 | SEMA5A | rs2135074 | 5 | 9486319 | G | A |
| 2415 | SEMA5A | rs1858428 | 5 | 9491537 | C | T |
| 2416 | SEMA5A | rs3822796 | 5 | 9495028 | C | T |
| 2417 | SEMA5A | rs268529 | 5 | 9495993 | T | G |
| 2418 | SEMA5A | rs1512508 | 5 | 9498460 | G | A |
| 2419 | SEMA5A | rs3798024 | 5 | 9500038 | A | G |
| 2420 | SEMA5A | rs268525 | 5 | 9500500 | G | A |
| 2421 | SEMA5A | rs3798028 | 5 | 9500716 | G | T |
| 2422 | SEMA5A | rs436243 | 5 | 9506088 | T | G |
| 2423 | SEMA5A | rs268542 | 5 | 9509165 | G | T |
| 2424 | SEMA5A | rs3798031 | 5 | 9510242 | C | T |
| 2425 | SEMA5A | rs3822803 | 5 | 9511878 | C | A |
| 2426 | SEMA5A | rs397287 | 5 | 9516290 | C | A |
| 2427 | SEMA5A | rs12657858 | 5 | 9521864 | G | A |
| 2428 | SEMA5A | rs418768 | 5 | 9535177 | A | G |
| 2429 | SEMA5A | rs418446 | 5 | 9536208 | C | T |
| 2430 | SEMA5A | rs950050 | 5 | 9541688 | A | G |
| 2431 | SEMA5A | rs268498 | 5 | 9546735 | A | T |
| 2432 | SEMA5A | rs421548 | 5 | 9561978 | C | T |
| 2433 | SEMA5A | rs11959773 | 5 | 9562810 | A | C |
| 2434 | SEMA5A | rs3798063 | 5 | 9572909 | C | T |
| 2435 | SEMA5A | rs3798064 | 5 | 9572977 | A | G |
| 2436 | SEMA5A | rs465107 | 5 | 9575302 | A | G |
| 2437 | SEMA5A | rs465991 | 5 | 9582243 | C | T |
| 2438 | SEMA5A | rs6881628 | 5 | 9592539 | T | G |
| 2439 | SEMA5A | rs3798089 | 5 | 9594288 | A | G |
| 2440 | SEMA5A | rs605126 | 5 | 9594865 | T | C |
| 2441 | SEMA5A | rs3798097 | 5 | 9595528 | C | T |
| 2442 | SEMA5A | rs1651282 | 5 | 9600020 | T | C |
| 2443 | SEMA5B | rs2303982 | 3 | 124113012 | G | T |
| 2444 | SEMA5B | rs2303983 | 3 | 124113035 | C | T |
| 2445 | SEMA5B | rs1011151 | 3 | 124121278 | G | A |
| 2446 | SEMA5B | rs4378918 | 3 | 124121698 | G | A |
| 2447 | SEMA5B | rs2276779 | 3 | 124125104 | G | C |
| 2448 | SEMA5B | rs2276778 | 3 | 124125279 | T | C |
| 2449 | SEMA5B | rs2276777 | 3 | 124127966 | G | A |
| 2450 | SEMA5B | rs2276774 | 3 | 124129517 | T | C |
| 2451 | SEMA5B | rs9873067 | 3 | 124132211 | G | A |
| 2452 | SEMA5B | rs13099843 | 3 | 124133579 | A | C |
| 2453 | SEMA5B | rs4677976 | 3 | 124138705 | C | A |
| 2454 | SEMA5B | rs10934622 | 3 | 124142518 | A | G |
| 2455 | SEMA5B | rs2043172 | 3 | 124145444 | A | G |
| 2456 | SEMA5B | rs4677868 | 3 | 124147541 | A | G |
| 2457 | SEMA5B | rs9831044 | 3 | 124148737 | G | A |
| 2458 | SEMA5B | rs4677983 | 3 | 124151846 | G | A |
| 2459 | SEMA5B | rs4478030 | 3 | 124161196 | A | G |
| 2460 | SEMA5B | rs4677985 | 3 | 124162860 | A | G |
| 2461 | SEMA5B | rs17171 | 3 | 124164619 | A | G |
| 2462 | SEMA5B | rs4677872 | 3 | 124166728 | G | A |
| 2463 | SEMA5B | rs6764971 | 3 | 124168802 | A | G |
| 2464 | SEMA5B | rs9816759 | 3 | 124170859 | A | C |
| 2465 | SEMA5B | rs895075 | 3 | 124177216 | A | G |
| 2466 | SEMA5B | rs9968193 | 3 | 124177619 | C | T |
| 2467 | SEMA5B | rs7432970 | 3 | 124177977 | G | A |
| 2468 | SEMA5B | rs7624272 | 3 | 124178240 | A | G |
| 2469 | SEMA5B | rs1470399 | 3 | 124181450 | C | T |
| 2470 | SEMA5B | rs1444820 | 3 | 124194033 | T | C |
| 2471 | SEMA5B | rs2120806 | 3 | 124198501 | C | T |
| 2472 | SEMA5B | rs9874307 | 3 | 124207630 | G | A |
| 2473 | SEMA5B | rs2034177 | 3 | 124211778 | A | G |
| 2474 | SEMA5B | rs9864897 | 3 | 124213404 | G | T |
| 2475 | SEMA5B | rs6804078 | 3 | 124224073 | G | A |
| 2476 | SEMA6A | rs3733724 | 5 | 115807871 | C | T |
| 2477 | SEMA6A | rs2303752 | 5 | 115813410 | C | T |
| 2478 | SEMA6A | rs2303753 | 5 | 115813420 | T | C |
| 2479 | SEMA6A | rs10447236 | 5 | 115815121 | G | T |
| 2480 | SEMA6A | rs26602 | 5 | 115816002 | G | A |
| 2481 | SEMA6A | rs1963858 | 5 | 115817497 | G | A |
| 2482 | SEMA6A | rs187710 | 5 | 115824360 | A | G |
| 2483 | SEMA6A | rs154555 | 5 | 115825750 | C | G |
| 2484 | SEMA6A | rs10077506 | 5 | 115828087 | G | A |
| 2485 | SEMA6A | rs28029 | 5 | 115828861 | A | G |
| 2486 | SEMA6A | rs27644 | 5 | 115829600 | T | C |
| 2487 | SEMA6A | rs258019 | 5 | 115835586 | A | G |
| 2488 | SEMA6A | rs423834 | 5 | 115839118 | A | G |
| 2489 | SEMA6A | rs3213797 | 5 | 115839417 | T | C |
| 2490 | SEMA6A | rs34967 | 5 | 115842004 | C | T |
| 2491 | SEMA6A | rs2278264 | 5 | 115844721 | G | A |
| 2492 | SEMA6A | rs2059874 | 5 | 115850290 | G | A |
| 2493 | SEMA6A | rs2287685 | 5 | 115850821 | G | C |
| 2494 | SEMA6A | rs154599 | 5 | 115855254 | A | G |
| 2495 | SEMA6A | rs1014643 | 5 | 115858367 | A | G |
| 2496 | SEMA6A | rs32972 | 5 | 115858990 | G | A |
| 2497 | SEMA6A | rs32973 | 5 | 115859203 | T | C |
| 2498 | SEMA6A | rs2287686 | 5 | 115859228 | T | C |
| 2499 | SEMA6A | rs32974 | 5 | 115859771 | G | A |
| 2500 | SEMA6A | rs41099 | 5 | 115859954 | G | A |
| 2501 | SEMA6A | rs258014 | 5 | 115861542 | G | T |
| 2502 | SEMA6A | rs2252892 | 5 | 115861751 | G | A |
| 2503 | SEMA6A | rs258015 | 5 | 115862331 | G | C |
| 2504 | SEMA6A | rs10519473 | 5 | 115863269 | A | G |
| 2505 | SEMA6A | rs194450 | 5 | 115866580 | C | G |
| 2506 | SEMA6A | rs154575 | 5 | 115868176 | T | G |
| 2507 | SEMA6A | rs918425 | 5 | 115873709 | A | C |
| 2508 | SEMA6A | rs6897947 | 5 | 115875944 | T | C |
| 2509 | SEMA6A | rs7728185 | 5 | 115878296 | G | A |
| 2510 | SEMA6A | rs7734164 | 5 | 115879809 | C | T |
| 2511 | SEMA6A | rs154577 | 5 | 115885389 | G | T |
| 2512 | SEMA6A | rs154576 | 5 | 115889530 | T | C |
| 2513 | SEMA6A | rs153645 | 5 | 115892476 | T | C |
| 2514 | SEMA6A | rs7734835 | 5 | 115897158 | C | T |
| 2515 | SEMA6A | rs254228 | 5 | 115913498 | T | C |
| 2516 | SEMA6A | rs254230 | 5 | 115913756 | A | G |
| 2517 | SEMA6A | rs153641 | 5 | 115919687 | G | A |
| 2518 | SEMA6A | rs10055012 | 5 | 115928561 | T | G |
| 2519 | SEMA6A | rs1862372 | 5 | 115938131 | T | C |
| 2520 | SEMA6A | rs3806914 | 5 | 115940394 | A | T |
| 2521 | SEMA6B | rs10426311 | 19 | 4496716 | C | T |
| 2522 | SEMA6B | rs4807602 | 19 | 4499014 | G | A |
| 2523 | SEMA6B | rs4807604 | 19 | 4504184 | G | A |
| 2524 | SEMA6B | rs1610094 | 19 | 4505582 | G | T |
| 2525 | SEMA6B | rs1610093 | 19 | 4505773 | A | G |
| 2526 | SEMA6B | rs7249087 | 19 | 4510645 | G | A |
| 2527 | SEMA6C | rs4971004 | 1 | 149370701 | C | T |
| 2528 | SEMA6C | rs4971005 | 1 | 149371221 | G | A |
| 2529 | SEMA6C | rs4971007 | 1 | 149374760 | G | T |
| 2530 | SEMA6C | rs4971008 | 1 | 149376264 | A | G |
| 2531 | SEMA6C | rs4971009 | 1 | 149383119 | C | T |
| 2532 | SEMA6C | rs11204782 | 1 | 149384658 | C | G |
| 2533 | SEMA6C | rs12087892 | 1 | 149385123 | T | C |
| 2534 | SEMA6D | rs514327 | 15 | 45811161 | T | G |
| 2535 | SEMA6D | rs586799 | 15 | 45812213 | A | G |
| 2536 | SEMA6D | rs687566 | 15 | 45818572 | G | A |
| 2537 | SEMA6D | rs11070610 | 15 | 45831978 | C | T |
| 2538 | SEMA6D | rs501916 | 15 | 45840520 | C | A |
| 2539 | SEMA6D | rs599111 | 15 | 45840625 | G | T |
| 2540 | SEMA6D | rs3743276 | 15 | 45842410 | T | C |
| 2541 | SEMA6D | rs565627 | 15 | 45844026 | T | G |
| 2542 | SEMA6D | rs537052 | 15 | 45844910 | A | G |
| 2543 | SEMA6D | rs532598 | 15 | 45845362 | C | T |
| 2544 | SEMA6D | rs616871 | 15 | 45845550 | A | T |
| 2545 | SEMA6D | rs8037600 | 15 | 45847622 | A | G |
| 2546 | SEMA6D | rs568215 | 15 | 45850367 | G | A |
| 2547 | SEMA7A | rs2075594 | 15 | 72489713 | A | G |
| 2548 | SEMA7A | rs741761 | 15 | 72490981 | A | G |
| 2549 | SEMA7A | rs11852686 | 15 | 72502257 | T | C |
| 2550 | SEMA7A | rs8036030 | 15 | 72503661 | G | A |
| 2551 | SEMA7A | rs1992145 | 15 | 72510696 | T | C |
| 2552 | SLIT1 | rs4917753 | 10 | 98756579 | C | A |
| 2553 | SLIT1 | rs11188985 | 10 | 98757831 | T | G |
| 2554 | SLIT1 | rs12254525 | 10 | 98764091 | T | C |
| 2555 | SLIT1 | rs3740527 | 10 | 98768466 | C | T |
| 2556 | SLIT1 | rs7896883 | 10 | 98777343 | T | C |
| 2557 | SLIT1 | rs7086186 | 10 | 98782414 | A | G |
| 2558 | SLIT1 | rs7089880 | 10 | 98782548 | A | G |
| 2559 | SLIT1 | rs7075194 | 10 | 98786462 | G | A |
| 2560 | SLIT1 | rs12244078 | 10 | 98792484 | C | G |
| 2561 | SLIT1 | rs2636770 | 10 | 98795601 | G | A |
| 2562 | SLIT1 | rs2805588 | 10 | 98798855 | G | C |
| 2563 | SLIT1 | rs902470 | 10 | 98800870 | G | A |
| 2564 | SLIT1 | rs2306951 | 10 | 98806288 | C | T |
| 2565 | SLIT1 | rs2817675 | 10 | 98806775 | C | T |
| 2566 | SLIT1 | rs2817676 | 10 | 98809306 | G | A |
| 2567 | SLIT1 | rs2306953 | 10 | 98810303 | G | A |
| 2568 | SLIT1 | rs2817677 | 10 | 98811817 | A | G |
| 2569 | SLIT1 | rs12773769 | 10 | 98822686 | A | G |
| 2570 | SLIT1 | rs2817683 | 10 | 98828683 | C | T |
| 2571 | SLIT1 | rs2817685 | 10 | 98835525 | A | G |
| 2572 | SLIT1 | rs2817688 | 10 | 98840789 | A | G |
| 2573 | SLIT1 | rs2817689 | 10 | 98847912 | T | C |
| 2574 | SLIT1 | rs2279508 | 10 | 98852730 | A | G |
| 2575 | SLIT1 | rs2279510 | 10 | 98853178 | C | T |
| 2576 | SLIT1 | rs2817695 | 10 | 98859377 | A | T |
| 2577 | SLIT1 | rs2817698 | 10 | 98861594 | A | G |
| 2578 | SLIT1 | rs2817701 | 10 | 98864102 | A | G |
| 2579 | SLIT1 | rs2636788 | 10 | 98866930 | A | G |
| 2580 | SLIT1 | rs902475 | 10 | 98872414 | G | A |
| 2581 | SLIT1 | rs1336258 | 10 | 98874391 | T | G |
| 2582 | SLIT1 | rs2817708 | 10 | 98879702 | C | T |
| 2583 | SLIT1 | rs2817649 | 10 | 98889144 | T | C |
| 2584 | SLIT1 | rs2817656 | 10 | 98895212 | A | G |
| 2585 | SLIT1 | rs2636809 | 10 | 98900752 | T | C |
| 2586 | SLIT1 | rs2784921 | 10 | 98902985 | C | T |
| 2587 | SLIT1 | rs2784920 | 10 | 98903617 | A | C |
| 2588 | SLIT1 | rs3000208 | 10 | 98911053 | C | A |
| 2589 | SLIT1 | rs2817661 | 10 | 98912748 | G | A |
| 2590 | SLIT1 | rs902468 | 10 | 98914210 | T | G |
| 2591 | SLIT1 | rs7073919 | 10 | 98916950 | A | G |
| 2592 | SLIT1 | rs4919077 | 10 | 98918560 | C | T |
| 2593 | SLIT1 | rs2636802 | 10 | 98920381 | G | A |
| 2594 | SLIT1 | rs2817666 | 10 | 98923842 | G | A |
| 2595 | SLIT1 | rs1565495 | 10 | 98928796 | T | G |
| 2596 | SLIT1 | rs902472 | 10 | 98932857 | A | T |
| 2597 | SLIT1 | rs3758587 | 10 | 98936233 | C | T |
| 2598 | SLIT2 | rs7655084 | 4 | 19864403 | T | G |
| 2599 | SLIT2 | rs2196476 | 4 | 19879697 | C | T |
| 2600 | SLIT2 | rs587032 | 4 | 19880870 | T | C |
| 2601 | SLIT2 | rs6447952 | 4 | 19885349 | G | A |
| 2602 | SLIT2 | rs7680377 | 4 | 19888955 | T | G |
| 2603 | SLIT2 | rs17601458 | 4 | 19892166 | C | T |
| 2604 | SLIT2 | rs6828506 | 4 | 19900020 | T | C |
| 2605 | SLIT2 | rs593943 | 4 | 19901336 | A | G |
| 2606 | SLIT2 | rs12233914 | 4 | 19906677 | A | G |
| 2607 | SLIT2 | rs624354 | 4 | 19909289 | C | T |
| 2608 | SLIT2 | rs13140329 | 4 | 19917019 | C | T |
| 2609 | SLIT2 | rs1323062 | 4 | 19918839 | G | A |
| 2610 | SLIT2 | rs13434430 | 4 | 19919270 | T | C |
| 2611 | SLIT2 | rs12646142 | 4 | 19927934 | T | C |
| 2612 | SLIT2 | rs6447955 | 4 | 19957036 | G | A |
| 2613 | SLIT2 | rs5016268 | 4 | 19979240 | A | G |
| 2614 | SLIT2 | rs13142826 | 4 | 20000712 | G | A |
| 2615 | SLIT2 | rs7435615 | 4 | 20008265 | G | A |
| 2616 | SLIT2 | rs9996157 | 4 | 20014129 | C | T |
| 2617 | SLIT2 | rs6813778 | 4 | 20025402 | A | G |
| 2618 | SLIT2 | rs6447961 | 4 | 20041522 | T | C |
| 2619 | SLIT2 | rs6849536 | 4 | 20049573 | C | T |
| 2620 | SLIT2 | rs884770 | 4 | 20056137 | A | G |
| 2621 | SLIT2 | rs12512717 | 4 | 20058862 | A | G |
| 2622 | SLIT2 | rs16869615 | 4 | 20063981 | T | C |
| 2623 | SLIT2 | rs9990844 | 4 | 20065052 | A | C |
| 2624 | SLIT2 | rs11733205 | 4 | 20067785 | T | C |
| 2625 | SLIT2 | rs11942897 | 4 | 20068215 | C | T |
| 2626 | SLIT2 | rs1033111 | 4 | 20076864 | C | T |
| 2627 | SLIT2 | rs7691135 | 4 | 20079490 | T | G |
| 2628 | SLIT2 | rs11936122 | 4 | 20082375 | G | T |
| 2629 | SLIT2 | rs1902845 | 4 | 20082896 | T | C |
| 2630 | SLIT2 | rs491795 | 4 | 20087381 | T | C |
| 2631 | SLIT2 | rs17612037 | 4 | 20093353 | A | G |
| 2632 | SLIT2 | rs569230 | 4 | 20098121 | T | C |
| 2633 | SLIT2 | rs3756147 | 4 | 20098576 | C | T |
| 2634 | SLIT2 | rs4443280 | 4 | 20099764 | G | A |
| 2635 | SLIT2 | rs522501 | 4 | 20106374 | T | C |
| 2636 | SLIT2 | rs490478 | 4 | 20107581 | G | A |
| 2637 | SLIT2 | rs729918 | 4 | 20115198 | G | A |
| 2638 | SLIT2 | rs6810790 | 4 | 20124767 | A | G |
| 2639 | SLIT2 | rs6447967 | 4 | 20125438 | T | C |
| 2640 | SLIT2 | rs6825654 | 4 | 20127700 | A | C |
| 2641 | SLIT2 | rs519813 | 4 | 20130090 | G | A |
| 2642 | SLIT2 | rs7690660 | 4 | 20136961 | T | G |
| 2643 | SLIT2 | rs3775815 | 4 | 20138767 | G | T |
| 2644 | SLIT2 | rs663032 | 4 | 20143088 | C | T |
| 2645 | SLIT2 | rs573118 | 4 | 20143783 | T | C |
| 2646 | SLIT2 | rs3775816 | 4 | 20144814 | T | C |
| 2647 | SLIT2 | rs3775820 | 4 | 20149637 | G | A |
| 2648 | SLIT2 | rs16869739 | 4 | 20152806 | A | G |
| 2649 | SLIT2 | rs7690492 | 4 | 20153281 | C | T |
| 2650 | SLIT2 | rs2292439 | 4 | 20156935 | G | A |
| 2651 | SLIT2 | rs636798 | 4 | 20161254 | T | G |
| 2652 | SLIT2 | rs575088 | 4 | 20166556 | A | C |
| 2653 | SLIT2 | rs11945956 | 4 | 20166861 | T | — |
| 2654 | SLIT2 | rs10516357 | 4 | 20172205 | G | A |
| 2655 | SLIT2 | rs506358 | 4 | 20174958 | C | T |
| 2656 | SLIT2 | rs2290750 | 4 | 20178196 | A | G |
| 2657 | SLIT2 | rs2290751 | 4 | 20178356 | C | T |
| 2658 | SLIT2 | rs644607 | 4 | 20180433 | C | T |
| 2659 | SLIT2 | rs7666974 | 4 | 20188469 | G | T |
| 2660 | SLIT2 | rs2168802 | 4 | 20194980 | C | T |
| 2661 | SLIT2 | rs9992591 | 4 | 20202843 | A | C |
| 2662 | SLIT2 | rs2322559 | 4 | 20214691 | A | G |
| 2663 | SLIT2 | rs1350400 | 4 | 20222727 | A | G |
| 2664 | SLIT2 | rs1379659 | 4 | 20229780 | A | G |
| 2665 | SLIT3 | rs12521041 | 5 | 168041433 | C | T |
| 2666 | SLIT3 | rs292488 | 5 | 168041836 | G | A |
| 2667 | SLIT3 | rs898401 | 5 | 168048441 | T | C |
| 2668 | SLIT3 | rs1864954 | 5 | 168055890 | T | C |
| 2669 | SLIT3 | rs6869359 | 5 | 168057576 | C | T |
| 2670 | SLIT3 | rs4868489 | 5 | 168066406 | A | G |
| 2671 | SLIT3 | rs1864952 | 5 | 168071816 | G | A |
| 2672 | SLIT3 | rs7715777 | 5 | 168079906 | T | C |
| 2673 | SLIT3 | rs11746295 | 5 | 168082336 | A | G |
| 2674 | SLIT3 | rs6890724 | 5 | 168096654 | G | A |
| 2675 | SLIT3 | rs10516049 | 5 | 168097246 | A | G |
| 2676 | SLIT3 | rs2337552 | 5 | 168099869 | C | T |
| 2677 | SLIT3 | rs4502845 | 5 | 168107617 | T | G |
| 2678 | SLIT3 | rs3822657 | 5 | 168107868 | T | C |
| 2679 | SLIT3 | rs2304035 | 5 | 168109094 | A | G |
| 2680 | SLIT3 | rs10060585 | 5 | 168111620 | T | C |
| 2681 | SLIT3 | rs10036727 | 5 | 168112658 | T | C |
| 2682 | SLIT3 | rs1549910 | 5 | 168113166 | A | G |
| 2683 | SLIT3 | rs7725943 | 5 | 168120735 | C | T |
| 2684 | SLIT3 | rs17070430 | 5 | 168121257 | G | A |
| 2685 | SLIT3 | rs2288796 | 5 | 168122078 | G | A |
| 2686 | SLIT3 | rs11743764 | 5 | 168125452 | G | A |
| 2687 | SLIT3 | rs1014194 | 5 | 168125521 | T | G |
| 2688 | SLIT3 | rs6860854 | 5 | 168126753 | T | C |
| 2689 | SLIT3 | rs9784690 | 5 | 168128611 | C | T |
| 2690 | SLIT3 | rs1368355 | 5 | 168129276 | G | C |
| 2691 | SLIT3 | rs10516050 | 5 | 168131878 | C | T |
| 2692 | SLIT3 | rs2288794 | 5 | 168132681 | T | C |
| 2693 | SLIT3 | rs871097 | 5 | 168140684 | T | C |
| 2694 | SLIT3 | rs2163761 | 5 | 168143831 | G | A |
| 2695 | SLIT3 | rs2292141 | 5 | 168145264 | G | A |
| 2696 | SLIT3 | rs891921 | 5 | 168145528 | C | T |
| 2697 | SLIT3 | rs2292143 | 5 | 168145584 | G | A |
| 2698 | SLIT3 | rs891928 | 5 | 168149344 | G | A |
| 2699 | SLIT3 | rs891927 | 5 | 168149456 | A | T |
| 2700 | SLIT3 | rs3797717 | 5 | 168149975 | A | T |
| 2701 | SLIT3 | rs3822666 | 5 | 168150045 | G | A |
| 2702 | SLIT3 | rs7728604 | 5 | 168152679 | C | T |
| 2703 | SLIT3 | rs11747556 | 5 | 168159407 | G | A |
| 2704 | SLIT3 | rs884787 | 5 | 168167094 | A | G |
| 2705 | SLIT3 | rs883241 | 5 | 168167414 | G | A |
| 2706 | SLIT3 | rs867477 | 5 | 168167568 | G | T |
| 2707 | SLIT3 | rs883242 | 5 | 168167757 | T | G |
| 2708 | SLIT3 | rs9885172 | 5 | 168174540 | T | C |
| 2709 | SLIT3 | rs1109105 | 5 | 168177603 | A | G |
| 2710 | SLIT3 | rs17635284 | 5 | 168178678 | A | G |
| 2711 | SLIT3 | rs17665948 | 5 | 168179662 | A | G |
| 2712 | SLIT3 | rs1875977 | 5 | 168183006 | T | C |
| 2713 | SLIT3 | rs7726584 | 5 | 168187474 | T | G |
| 2714 | SLIT3 | rs4282339 | 5 | 168188817 | G | A |
| 2715 | SLIT3 | rs949780 | 5 | 168190700 | C | T |
| 2716 | SLIT3 | rs1394618 | 5 | 168192793 | C | T |
| 2717 | SLIT3 | rs2974428 | 5 | 168197821 | C | T |
| 2718 | SLIT3 | rs1979923 | 5 | 168198468 | T | C |
| 2719 | SLIT3 | rs1875969 | 5 | 168201890 | G | C |
| 2720 | SLIT3 | rs2938767 | 5 | 168212966 | A | G |
| 2721 | SLIT3 | rs6555834 | 5 | 168218793 | T | C |
| 2722 | SLIT3 | rs1875972 | 5 | 168221569 | G | A |
| 2723 | SLIT3 | rs2938769 | 5 | 168225316 | C | T |
| 2724 | SLIT3 | rs1466947 | 5 | 168231235 | A | C |
| 2725 | SLIT3 | rs2938772 | 5 | 168239624 | G | A |
| 2726 | SLIT3 | rs1553536 | 5 | 168242669 | G | T |
| 2727 | SLIT3 | rs7707709 | 5 | 168246008 | A | G |
| 2728 | SLIT3 | rs4616916 | 5 | 168246138 | C | G |
| 2729 | SLIT3 | rs4616917 | 5 | 168246538 | C | T |
| 2730 | SLIT3 | rs11134544 | 5 | 168252633 | G | T |
| 2731 | SLIT3 | rs10516053 | 5 | 168263028 | T | C |
| 2732 | SLIT3 | rs10069222 | 5 | 168263915 | G | A |
| 2733 | SLIT3 | rs7709047 | 5 | 168270631 | G | T |
| 2734 | SLIT3 | rs10063042 | 5 | 168285385 | G | A |
| 2735 | SLIT3 | rs1982062 | 5 | 168288224 | G | A |
| 2736 | SLIT3 | rs6879973 | 5 | 168295767 | C | T |
| 2737 | SLIT3 | rs919753 | 5 | 168296181 | G | A |
| 2738 | SLIT3 | rs11134553 | 5 | 168305255 | A | C |
| 2739 | SLIT3 | rs10042354 | 5 | 168306548 | A | G |
| 2740 | SLIT3 | rs6555837 | 5 | 168308871 | C | T |
| 2741 | SLIT3 | rs7703544 | 5 | 168313531 | T | G |
| 2742 | SLIT3 | rs10054320 | 5 | 168318771 | A | G |
| 2743 | SLIT3 | rs6555842 | 5 | 168320021 | C | T |
| 2744 | SLIT3 | rs1432910 | 5 | 168324134 | A | G |
| 2745 | SLIT3 | rs6555845 | 5 | 168344146 | C | T |
| 2746 | SLIT3 | rs11747642 | 5 | 168356484 | T | C |
| 2747 | SLIT3 | rs10062450 | 5 | 168362983 | A | G |
| 2748 | SLIT3 | rs12152726 | 5 | 168365990 | T | C |
| 2749 | SLIT3 | rs2163783 | 5 | 168372053 | G | A |
| 2750 | SLIT3 | rs1432906 | 5 | 168378947 | G | A |
| 2751 | SLIT3 | rs6890327 | 5 | 168385054 | C | A |
| 2752 | SLIT3 | rs2879135 | 5 | 168387514 | A | G |
| 2753 | SLIT3 | rs1432899 | 5 | 168391546 | C | T |
| 2754 | SLIT3 | rs7447181 | 5 | 168391829 | C | T |
| 2755 | SLIT3 | rs2278386 | 5 | 168397803 | G | A |
| 2756 | SLIT3 | rs7717558 | 5 | 168402555 | G | T |
| 2757 | SLIT3 | rs7737524 | 5 | 168411729 | C | T |
| 2758 | SLIT3 | rs7735291 | 5 | 168416171 | G | A |
| 2759 | SLIT3 | rs13154825 | 5 | 168417071 | A | C |
| 2760 | SLIT3 | rs10516057 | 5 | 168418547 | T | C |
| 2761 | SLIT3 | rs882015 | 5 | 168418870 | G | T |
| 2762 | SLIT3 | rs1469683 | 5 | 168421910 | A | G |
| 2763 | SLIT3 | rs4868223 | 5 | 168423757 | A | G |
| 2764 | SLIT3 | rs7701998 | 5 | 168427718 | A | C |
| 2765 | SLIT3 | rs2338296 | 5 | 168428921 | T | C |
| 2766 | SLIT3 | rs6864266 | 5 | 168431254 | T | C |
| 2767 | SLIT3 | rs188433 | 5 | 168434906 | T | C |
| 2768 | SLIT3 | rs296009 | 5 | 168445761 | G | T |
| 2769 | SLIT3 | rs296007 | 5 | 168446205 | T | C |
| 2770 | SLIT3 | rs296006 | 5 | 168447444 | G | A |
| 2771 | SLIT3 | rs296002 | 5 | 168454445 | G | T |
| 2772 | SLIT3 | rs295999 | 5 | 168458444 | G | A |
| 2773 | SLIT3 | rs295996 | 5 | 168465044 | A | G |
| 2774 | SLIT3 | rs2569114 | 5 | 168471817 | C | T |
| 2775 | SLIT3 | rs297808 | 5 | 168483142 | A | G |
| 2776 | SLIT3 | rs17070834 | 5 | 168486833 | T | C |
| 2777 | SLIT3 | rs2668046 | 5 | 168491380 | G | A |
| 2778 | SLIT3 | rs888688 | 5 | 168492756 | T | C |
| 2779 | SLIT3 | rs2338297 | 5 | 168497135 | T | C |
| 2780 | SLIT3 | rs12522564 | 5 | 168503023 | G | A |
| 2781 | SLIT3 | rs297836 | 5 | 168503882 | G | A |
| 2782 | SLIT3 | rs297832 | 5 | 168508528 | C | T |
| 2783 | SLIT3 | rs6884996 | 5 | 168509506 | C | T |
| 2784 | SLIT3 | rs297827 | 5 | 168512279 | G | A |
| 2785 | SLIT3 | rs297812 | 5 | 168516543 | A | G |
| 2786 | SLIT3 | rs7722860 | 5 | 168521503 | G | A |
| 2787 | SLIT3 | rs297823 | 5 | 168527580 | C | T |
| 2788 | SLIT3 | rs9313446 | 5 | 168530764 | G | A |
| 2789 | SLIT3 | rs10516061 | 5 | 168532206 | G | A |
| 2790 | SLIT3 | rs1006329 | 5 | 168532428 | A | G |
| 2791 | SLIT3 | rs297879 | 5 | 168543365 | C | A |
| 2792 | SLIT3 | rs9313447 | 5 | 168544309 | G | A |
| 2793 | SLIT3 | rs297883 | 5 | 168545253 | G | A |
| 2794 | SLIT3 | rs297884 | 5 | 168545412 | A | C |
| 2795 | SLIT3 | rs7733887 | 5 | 168551432 | A | G |
| 2796 | SLIT3 | rs177074 | 5 | 168551973 | C | T |
| 2797 | SLIT3 | rs297838 | 5 | 168554350 | C | T |
| 2798 | SLIT3 | rs17643060 | 5 | 168565606 | T | C |
| 2799 | SLIT3 | rs1345592 | 5 | 168573567 | A | G |
| 2800 | SLIT3 | rs4868388 | 5 | 168575141 | A | C |
| 2801 | SLIT3 | rs716866 | 5 | 168578300 | T | C |
| 2802 | SLIT3 | rs978902 | 5 | 168582773 | C | T |
| 2803 | SLIT3 | rs6893511 | 5 | 168586690 | C | A |
| 2804 | SLIT3 | rs10079601 | 5 | 168590743 | T | C |
| 2805 | SLIT3 | rs10038332 | 5 | 168605983 | T | C |
| 2806 | SLIT3 | rs972031 | 5 | 168610370 | C | T |
| 2807 | SLIT3 | rs11954621 | 5 | 168612480 | G | A |
| 2808 | SLIT3 | rs7716873 | 5 | 168613886 | G | A |
| 2809 | SLIT3 | rs11134564 | 5 | 168621227 | A | G |
| 2810 | SLIT3 | rs754431 | 5 | 168636528 | G | A |
| 2811 | SLIT3 | rs11134568 | 5 | 168640139 | C | T |
| 2812 | SLIT3 | rs888781 | 5 | 168641644 | C | T |
| 2813 | SLIT3 | rs888783 | 5 | 168655236 | G | A |
| 2814 | SLIT3 | rs7380303 | 5 | 168655514 | T | C |
| 2815 | SRGAP1 | rs11175189 | 12 | 62532341 | A | C |
| 2816 | SRGAP1 | rs7304340 | 12 | 62549194 | T | C |
| 2817 | SRGAP1 | rs7966445 | 12 | 62552193 | A | G |
| 2818 | SRGAP1 | rs7953208 | 12 | 62554271 | C | T |
| 2819 | SRGAP1 | rs10878092 | 12 | 62560946 | G | T |
| 2820 | SRGAP1 | rs4346062 | 12 | 62570549 | A | G |
| 2821 | SRGAP1 | rs2335882 | 12 | 62593702 | C | T |
| 2822 | SRGAP1 | rs11175208 | 12 | 62602703 | T | C |
| 2823 | SRGAP1 | rs9668434 | 12 | 62604999 | G | T |
| 2824 | SRGAP1 | rs3911400 | 12 | 62609763 | A | T |
| 2825 | SRGAP1 | rs11175211 | 12 | 62611920 | G | A |
| 2826 | SRGAP1 | rs1989584 | 12 | 62619478 | G | T |
| 2827 | SRGAP1 | rs10784374 | 12 | 62622044 | A | G |
| 2828 | SRGAP1 | rs10878096 | 12 | 62627371 | A | G |
| 2829 | SRGAP1 | rs1350136 | 12 | 62637172 | G | A |
| 2830 | SRGAP1 | rs3741607 | 12 | 62669936 | T | C |
| 2831 | SRGAP1 | rs1350140 | 12 | 62673595 | G | A |
| 2832 | SRGAP1 | rs7311377 | 12 | 62690200 | G | T |
| 2833 | SRGAP1 | rs6581522 | 12 | 62701296 | A | G |
| 2834 | SRGAP1 | rs4436640 | 12 | 62719724 | G | C |
| 2835 | SRGAP1 | rs11175238 | 12 | 62719998 | A | C |
| 2836 | SRGAP1 | rs10878102 | 12 | 62720280 | A | G |
| 2837 | SRGAP1 | rs11175240 | 12 | 62728701 | C | A |
| 2838 | SRGAP1 | rs990309 | 12 | 62734562 | T | C |
| 2839 | SRGAP1 | rs789716 | 12 | 62740754 | G | A |
| 2840 | SRGAP1 | rs1827073 | 12 | 62744467 | T | G |
| 2841 | SRGAP1 | rs789709 | 12 | 62757573 | G | C |
| 2842 | SRGAP1 | rs11175250 | 12 | 62761279 | T | C |
| 2843 | SRGAP1 | rs7980267 | 12 | 62770299 | T | C |
| 2844 | SRGAP1 | rs1874314 | 12 | 62770980 | C | T |
| 2845 | SRGAP1 | rs4763138 | 12 | 62792577 | G | A |
| 2846 | SRGAP1 | rs789722 | 12 | 62806072 | C | T |
| 2847 | SRGAP1 | rs10878106 | 12 | 62807833 | C | G |
| 2848 | SRGAP2 | rs9660435 | 1 | 204654544 | T | A |
| 2849 | SRGAP2 | rs11120040 | 1 | 204667948 | C | T |
| 2850 | SRGAP2 | rs2297539 | 1 | 204670157 | G | A |
| 2851 | SRGAP2 | rs4845104 | 1 | 204675942 | T | C |
| 2852 | SRGAP2 | rs2483064 | 1 | 204681394 | T | C |
| 2853 | SRGAP2 | rs2483062 | 1 | 204684304 | C | T |
| 2854 | SRGAP2 | rs2336938 | 1 | 204685421 | C | A |
| 2855 | SRGAP2 | rs2483060 | 1 | 204690574 | G | A |
| 2856 | SRGAP2 | rs3860302 | 1 | 204691265 | C | G |
| 2857 | SRGAP2 | rs11120575 | 1 | 204693229 | A | G |
| 2858 | SRGAP2 | rs12130290 | 1 | 204693799 | G | A |
| 2859 | SRGAP2 | rs3813982 | 1 | 204696296 | T | C |
| 2860 | SRGAP2 | rs7549189 | 1 | 204701917 | T | G |
| 2861 | SRGAP3 | rs341792 | 3 | 8998218 | T | C |
| 2862 | SRGAP3 | rs341791 | 3 | 8999016 | G | A |
| 2863 | SRGAP3 | rs341790 | 3 | 8999145 | A | C |
| 2864 | SRGAP3 | rs1043951 | 3 | 8999520 | T | C |
| 2865 | SRGAP3 | rs186889 | 3 | 9001817 | C | T |
| 2866 | SRGAP3 | rs413789 | 3 | 9009144 | C | A |
| 2867 | SRGAP3 | rs511815 | 3 | 9009557 | A | G |
| 2868 | SRGAP3 | rs523684 | 3 | 9014468 | G | A |
| 2869 | SRGAP3 | rs341788 | 3 | 9025658 | T | C |
| 2870 | SRGAP3 | rs932145 | 3 | 9025962 | A | T |
| 2871 | SRGAP3 | rs6443217 | 3 | 9037145 | A | G |
| 2872 | SRGAP3 | rs421041 | 3 | 9041852 | G | A |
| 2873 | SRGAP3 | rs370477 | 3 | 9042596 | T | C |
| 2874 | SRGAP3 | rs1703128 | 3 | 9043576 | A | G |
| 2875 | SRGAP3 | rs1703127 | 3 | 9043857 | T | C |
| 2876 | SRGAP3 | rs10510396 | 3 | 9044872 | A | G |
| 2877 | SRGAP3 | rs370526 | 3 | 9045494 | G | A |
| 2878 | SRGAP3 | rs6777129 | 3 | 9049842 | T | C |
| 2879 | SRGAP3 | rs6762129 | 3 | 9049864 | C | T |
| 2880 | SRGAP3 | rs3755784 | 3 | 9051136 | C | A |
| 2881 | SRGAP3 | rs3732977 | 3 | 9052449 | A | G |
| 2882 | SRGAP3 | rs557377 | 3 | 9053119 | G | A |
| 2883 | SRGAP3 | rs2670009 | 3 | 9053200 | T | G |
| 2884 | SRGAP3 | rs2271205 | 3 | 9054448 | C | G |
| 2885 | SRGAP3 | rs884237 | 3 | 9054895 | T | C |
| 2886 | SRGAP3 | rs2670008 | 3 | 9057850 | T | C |
| 2887 | SRGAP3 | rs667714 | 3 | 9060122 | C | T |
| 2888 | SRGAP3 | rs2244864 | 3 | 9064253 | C | G |
| 2889 | SRGAP3 | rs611406 | 3 | 9065904 | G | A |
| 2890 | SRGAP3 | rs610457 | 3 | 9066150 | C | G |
| 2891 | SRGAP3 | rs2662090 | 3 | 9066945 | A | C |
| 2892 | SRGAP3 | rs2664098 | 3 | 9073422 | T | C |
| 2893 | SRGAP3 | rs2664095 | 3 | 9074652 | G | A |
| 2894 | SRGAP3 | rs7644129 | 3 | 9076596 | C | A |
| 2895 | SRGAP3 | rs2669995 | 3 | 9081892 | G | A |
| 2896 | SRGAP3 | rs6443220 | 3 | 9081967 | T | C |
| 2897 | SRGAP3 | rs1162395 | 3 | 9086220 | C | T |
| 2898 | SRGAP3 | rs2675173 | 3 | 9089326 | G | A |
| 2899 | SRGAP3 | rs6793348 | 3 | 9089758 | T | C |
| 2900 | SRGAP3 | rs3887568 | 3 | 9092544 | G | A |
| 2901 | SRGAP3 | rs9825026 | 3 | 9092787 | A | G |
| 2902 | SRGAP3 | rs2669989 | 3 | 9095003 | G | A |
| 2903 | SRGAP3 | rs2664079 | 3 | 9106598 | C | A |
| 2904 | SRGAP3 | rs2664076 | 3 | 9108970 | C | T |
| 2905 | SRGAP3 | rs386369 | 3 | 9117834 | C | G |
| 2906 | SRGAP3 | rs386423 | 3 | 9117889 | C | T |
| 2907 | SRGAP3 | rs1861941 | 3 | 9122177 | A | G |
| 2908 | SRGAP3 | rs369068 | 3 | 9128129 | C | T |
| 2909 | SRGAP3 | rs391923 | 3 | 9131032 | A | G |
| 2910 | SRGAP3 | rs2543491 | 3 | 9135464 | A | G |
| 2911 | SRGAP3 | rs6781236 | 3 | 9138205 | G | A |
| 2912 | SRGAP3 | rs2077115 | 3 | 9140839 | A | G |
| 2913 | SRGAP3 | rs2287492 | 3 | 9147577 | A | G |
| 2914 | SRGAP3 | rs1300250 | 3 | 9147944 | C | A |
| 2915 | SRGAP3 | rs2675188 | 3 | 9151073 | C | T |
| 2916 | SRGAP3 | rs2675187 | 3 | 9154970 | T | G |
| 2917 | SRGAP3 | rs2668361 | 3 | 9156402 | G | A |
| 2918 | SRGAP3 | rs2257056 | 3 | 9163614 | T | G |
| 2919 | SRGAP3 | rs2675186 | 3 | 9168323 | G | A |
| 2920 | SRGAP3 | rs2675181 | 3 | 9172230 | A | G |
| 2921 | SRGAP3 | rs17530219 | 3 | 9173119 | T | C |
| 2922 | SRGAP3 | rs17530358 | 3 | 9175374 | G | A |
| 2923 | SRGAP3 | rs2664108 | 3 | 9182124 | C | T |
| 2924 | SRGAP3 | rs986119 | 3 | 9186355 | A | C |
| 2925 | SRGAP3 | rs446975 | 3 | 9192382 | G | T |
| 2926 | SRGAP3 | rs400052 | 3 | 9193726 | C | T |
| 2927 | SRGAP3 | rs802792 | 3 | 9199174 | G | A |
| 2928 | SRGAP3 | rs1154386 | 3 | 9204432 | T | C |
| 2929 | SRGAP3 | rs10490820 | 3 | 9205005 | G | A |
| 2930 | SRGAP3 | rs1154389 | 3 | 9207361 | G | A |
| 2931 | SRGAP3 | rs1145153 | 3 | 9209762 | G | A |
| 2932 | SRGAP3 | rs6776837 | 3 | 9210956 | A | G |
| 2933 | SRGAP3 | rs1145154 | 3 | 9214975 | C | A |
| 2934 | SRGAP3 | rs6781447 | 3 | 9215886 | C | T |
| 2935 | SRGAP3 | rs1145157 | 3 | 9216748 | C | T |
| 2936 | SRGAP3 | rs1145158 | 3 | 9220528 | C | T |
| 2937 | SRGAP3 | rs1642756 | 3 | 9223378 | A | G |
| 2938 | SRGAP3 | rs6797462 | 3 | 9225652 | C | T |
| 2939 | SRGAP3 | rs390151 | 3 | 9230417 | G | A |
| 2940 | SRGAP3 | rs4684627 | 3 | 9231397 | T | C |
| 2941 | SRGAP3 | rs1710903 | 3 | 9232979 | A | G |
| 2942 | SRGAP3 | rs1995994 | 3 | 9237649 | G | A |
| 2943 | SRGAP3 | rs1028154 | 3 | 9240619 | G | A |
| 2944 | SRGAP3 | rs2700331 | 3 | 9241055 | G | A |
| 2945 | SRGAP3 | rs2700326 | 3 | 9246858 | A | G |
| 2946 | SRGAP3 | rs938116 | 3 | 9251082 | A | C |
| 2947 | SRGAP3 | rs2137215 | 3 | 9253829 | A | G |
| 2948 | SRGAP3 | rs2875427 | 3 | 9258298 | T | C |
| 2949 | SRGAP3 | rs4684630 | 3 | 9262143 | A | C |
| 2950 | SRGAP3 | rs2137216 | 3 | 9263544 | T | C |
| 2951 | SRGAP3 | rs2600174 | 3 | 9264085 | C | A |
| 2952 | SRGAP3 | rs11131163 | 3 | 9266860 | A | G |
| 2953 | STK23 | rs762650 | X | 152698395 | G | A |
| 2954 | UNC5A | rs692653 | 5 | 176172371 | C | T |
| 2955 | UNC5A | rs2940520 | 5 | 176174930 | A | G |
| 2956 | UNC5A | rs12521024 | 5 | 176195032 | G | A |
| 2957 | UNC5A | rs480782 | 5 | 176205975 | T | C |
| 2958 | UNC5A | rs7380999 | 5 | 176226308 | T | C |
| 2959 | UNC5A | rs11954962 | 5 | 176227500 | G | T |
| 2960 | UNC5A | rs2304517 | 5 | 176237004 | C | T |
| 2961 | UNC5A | rs2304516 | 5 | 176238071 | T | A |
| 2962 | UNC5A | rs478253 | 5 | 176238283 | G | A |
| 2963 | UNC5A | rs3736432 | 5 | 176240109 | G | A |
| 2964 | UNC5B | rs10762430 | 10 | 72644933 | T | G |
| 2965 | UNC5B | rs12264668 | 10 | 72645439 | C | T |
| 2966 | UNC5B | rs2244140 | 10 | 72652259 | T | C |
| 2967 | UNC5B | rs875001 | 10 | 72654702 | T | C |
| 2968 | UNC5B | rs7893421 | 10 | 72661390 | C | T |
| 2969 | UNC5B | rs10999739 | 10 | 72668239 | C | T |
| 2970 | UNC5B | rs2894147 | 10 | 72670948 | A | G |
| 2971 | UNC5B | rs7899034 | 10 | 72680069 | G | A |
| 2972 | UNC5B | rs7915759 | 10 | 72680327 | T | G |
| 2973 | UNC5B | rs7907878 | 10 | 72682294 | G | A |
| 2974 | UNC5B | rs2394784 | 10 | 72689764 | G | A |
| 2975 | UNC5B | rs7902476 | 10 | 72693741 | C | A |
| 2976 | UNC5B | rs11598330 | 10 | 72704958 | G | T |
| 2977 | UNC5B | rs11593534 | 10 | 72713021 | T | C |
| 2978 | UNC5B | rs6415905 | 10 | 72715289 | G | C |
| 2979 | UNC5B | rs2277259 | 10 | 72717695 | A | G |
| 2980 | UNC5B | rs10762435 | 10 | 72717902 | G | A |
| 2981 | UNC5B | rs3740462 | 10 | 72720988 | A | G |
| 2982 | UNC5B | rs3740460 | 10 | 72721393 | A | G |
| 2983 | UNC5B | rs1105174 | 10 | 72725854 | G | A |
| 2984 | UNC5B | rs1060184 | 10 | 72729363 | C | G |
| 2985 | UNC5C | rs2241743 | 4 | 96310546 | T | C |
| 2986 | UNC5C | rs2289043 | 4 | 96325344 | C | T |
| 2987 | UNC5C | rs3755880 | 4 | 96327585 | C | T |
| 2988 | UNC5C | rs3775059 | 4 | 96336252 | A | C |
| 2989 | UNC5C | rs3775053 | 4 | 96342488 | G | A |
| 2990 | UNC5C | rs3733212 | 4 | 96343003 | A | G |
| 2991 | UNC5C | rs3775044 | 4 | 96345990 | C | T |
| 2992 | UNC5C | rs2276322 | 4 | 96346891 | C | A |
| 2993 | UNC5C | rs1865378 | 4 | 96353861 | C | A |
| 2994 | UNC5C | rs995062 | 4 | 96356534 | G | A |
| 2995 | UNC5C | rs2571037 | 4 | 96357205 | T | C |
| 2996 | UNC5C | rs7653969 | 4 | 96362573 | T | C |
| 2997 | UNC5C | rs7676599 | 4 | 96362722 | G | C |
| 2998 | UNC5C | rs2865384 | 4 | 96364142 | T | C |
| 2999 | UNC5C | rs6850673 | 4 | 96366882 | T | C |
| 3000 | UNC5C | rs10516960 | 4 | 96378699 | T | G |
| 3001 | UNC5C | rs4699412 | 4 | 96379139 | T | C |
| 3002 | UNC5C | rs11730446 | 4 | 96382266 | A | G |
| 3003 | UNC5C | rs2865414 | 4 | 96387741 | A | G |
| 3004 | UNC5C | rs3775003 | 4 | 96390233 | G | A |
| 3005 | UNC5C | rs4254782 | 4 | 96400056 | A | G |
| 3006 | UNC5C | rs2865416 | 4 | 96412721 | G | T |
| 3007 | UNC5C | rs10213145 | 4 | 96416959 | A | G |
| 3008 | UNC5C | rs6820369 | 4 | 96421244 | A | G |
| 3009 | UNC5C | rs10516962 | 4 | 96422400 | G | A |
| 3010 | UNC5C | rs2865423 | 4 | 96423795 | A | G |
| 3011 | UNC5C | rs6857589 | 4 | 96432905 | C | T |
| 3012 | UNC5C | rs4699850 | 4 | 96444976 | A | C |
| 3013 | UNC5C | rs1483735 | 4 | 96454543 | G | A |
| 3014 | UNC5C | rs2621439 | 4 | 96462026 | C | A |
| 3015 | UNC5C | rs2170780 | 4 | 96463263 | A | G |
| 3016 | UNC5C | rs10002089 | 4 | 96465483 | G | A |
| 3017 | UNC5C | rs2626053 | 4 | 96474110 | C | T |
| 3018 | UNC5C | rs10516965 | 4 | 96474836 | T | C |
| 3019 | UNC5C | rs1843018 | 4 | 96475695 | T | C |
| 3020 | UNC5C | rs1552225 | 4 | 96479864 | A | G |
| 3021 | UNC5C | rs2626050 | 4 | 96480371 | A | T |
| 3022 | UNC5C | rs2626074 | 4 | 96498746 | C | A |
| 3023 | UNC5C | rs2621456 | 4 | 96517597 | A | G |
| 3024 | UNC5C | rs4699264 | 4 | 96517749 | A | T |
| 3025 | UNC5C | rs10461127 | 4 | 96523030 | C | T |
| 3026 | UNC5C | rs7674643 | 4 | 96523190 | T | C |
| 3027 | UNC5C | rs17437679 | 4 | 96524606 | C | A |
| 3028 | UNC5C | rs1384862 | 4 | 96528233 | T | C |
| 3029 | UNC5C | rs10516969 | 4 | 96529185 | G | A |
| 3030 | UNC5C | rs10516970 | 4 | 96529258 | C | T |
| 3031 | UNC5C | rs1483739 | 4 | 96533282 | G | T |
| 3032 | UNC5C | rs4406027 | 4 | 96541826 | T | C |
| 3033 | UNC5C | rs7691901 | 4 | 96549439 | T | G |
| 3034 | UNC5C | rs1384863 | 4 | 96557855 | G | A |
| 3035 | UNC5C | rs10516971 | 4 | 96560068 | A | G |
| 3036 | UNC5C | rs9307159 | 4 | 96560908 | C | T |
| 3037 | UNC5C | rs900617 | 4 | 96563289 | G | A |
| 3038 | UNC5C | rs1483749 | 4 | 96568442 | G | A |
| 3039 | UNC5C | rs1483748 | 4 | 96572195 | C | T |
| 3040 | UNC5C | rs1384866 | 4 | 96573343 | A | G |
| 3041 | UNC5C | rs1026926 | 4 | 96575556 | C | T |
| 3042 | UNC5C | rs3846457 | 4 | 96579664 | T | C |
| 3043 | UNC5C | rs265015 | 4 | 96579818 | G | A |
| 3044 | UNC5C | rs9307160 | 4 | 96586976 | T | C |
| 3045 | UNC5C | rs265063 | 4 | 96587444 | T | C |
| 3046 | UNC5C | rs9995935 | 4 | 96588758 | G | A |
| 3047 | UNC5C | rs265047 | 4 | 96592702 | A | G |
| 3048 | UNC5C | rs265044 | 4 | 96593142 | T | C |
| 3049 | UNC5C | rs265043 | 4 | 96594188 | G | A |
| 3050 | UNC5C | rs265042 | 4 | 96594840 | A | G |
| 3051 | UNC5C | rs265039 | 4 | 96598248 | T | C |
| 3052 | UNC5C | rs3843446 | 4 | 96599295 | T | C |
| 3053 | UNC5C | rs4444836 | 4 | 96607673 | T | C |
| 3054 | UNC5C | rs6832346 | 4 | 96611273 | C | T |
| 3055 | UNC5C | rs265060 | 4 | 96612587 | T | C |
| 3056 | UNC5C | rs265070 | 4 | 96620669 | A | G |
| 3057 | UNC5C | rs4505826 | 4 | 96625972 | C | T |
| 3058 | UNC5C | rs6532561 | 4 | 96635494 | C | T |
| 3059 | UNC5C | rs7699716 | 4 | 96648014 | A | G |
| 3060 | UNC5C | rs10004953 | 4 | 96653229 | C | T |
| 3061 | UNC5C | rs17024125 | 4 | 96666526 | A | G |
| 3062 | UNC5C | rs1564896 | 4 | 96667709 | A | G |
| 3063 | UNC5C | rs1385669 | 4 | 96677874 | G | A |
| 3064 | UNC5C | rs10516978 | 4 | 96680436 | G | A |
| 3065 | UNC5C | rs7686209 | 4 | 96683219 | G | A |
| 3066 | UNC5C | rs1564895 | 4 | 96686399 | G | A |
| 3067 | UNC5C | rs998065 | 4 | 96687408 | G | A |
| 3068 | UNC5C | rs4699431 | 4 | 96688730 | A | T |
| 3069 | UNC5D | rs7002810 | 8 | 35521154 | C | T |
| 3070 | UNC5D | rs7833633 | 8 | 35529966 | A | G |
| 3071 | UNC5D | rs6989128 | 8 | 35531299 | G | A |
| 3072 | UNC5D | rs6980691 | 8 | 35531939 | T | G |
| 3073 | UNC5D | rs983271 | 8 | 35534193 | T | C |
| 3074 | UNC5D | rs983273 | 8 | 35534271 | T | G |
| 3075 | UNC5D | rs2579896 | 8 | 35543088 | T | C |
| 3076 | UNC5D | rs2589338 | 8 | 35548614 | C | T |
| 3077 | UNC5D | rs2067700 | 8 | 35552624 | C | T |
| 3078 | UNC5D | rs2589746 | 8 | 35555577 | T | C |
| 3079 | UNC5D | rs2579903 | 8 | 35561854 | T | C |
| 3080 | UNC5D | rs17353731 | 8 | 35599009 | A | G |
| 3081 | UNC5D | rs13250548 | 8 | 35627941 | A | G |
| 3082 | UNC5D | rs7845368 | 8 | 35631935 | T | G |
| 3083 | UNC5D | rs1946745 | 8 | 35642313 | C | T |
| 3084 | UNC5D | rs1898259 | 8 | 35644422 | G | A |
| 3085 | UNC5D | rs10112630 | 8 | 35654667 | G | A |
| 3086 | UNC5D | rs4739422 | 8 | 35687222 | T | C |
| 3087 | UNC5D | rs6983275 | 8 | 35703233 | T | C |
| 3088 | UNC5D | rs6996645 | 8 | 35703356 | G | C |
| 3089 | UNC5D | rs7007342 | 8 | 35710478 | C | T |
| 3090 | UNC5D | rs1436613 | 8 | 35716696 | A | G |
| 3091 | UNC5D | rs6468329 | 8 | 35727915 | T | C |
| 3092 | UNC5D | rs6468336 | 8 | 35732601 | T | C |
| 3093 | UNC5D | rs1347609 | 8 | 35749643 | T | G |
| 3094 | UNC5D | rs7836205 | 8 | 35761483 | A | G |
| 3095 | UNC5D | rs13273386 | 8 | 35765532 | T | C |

TABLE 17
SNPs in Axon Guidance Pathway Genes Predicting PD Susceptibility.

| Gene* (rs ID), | Odds Ratio | | | Odds Ratio | |
|---|---|---|---|---|---|
| Final Coding Scheme^{†} | (95% CI)** | P value** | Interaction* | (95% CI)** | P value** |
| ABLIM2 (rs3814062), a | 0.27 (0.139-0.53) | 1.1 × 10⁻⁰⁴ | DCC₂*PLXNA2₂ | 0.03 (0.003-0.25) | 1.3 × 10⁻⁰³ |
| CDC42 (rs2056975), d | 6.01 (1.854-19.46) | 2.8 × 10⁻⁰³ | SEMA3E₂*GSK3B | 74.96 (7.116-789.65) | 3.3 × 10⁻⁰⁴ |
| DCC₁ (rs7506794), a | 0.10 (0.025-0.42) | 1.5 × 10⁻⁰³ | | | |
| DCC₂ (rs1466373), d | 28.48 (6.346-127.85) | 1.2 × 10⁻⁰⁵ | | | |
| EFNA5 (rs152580), r | 4.43 (1.528-12.83) | 6.1 × 10⁻⁰³ | | | |
| EFNB2 (rs2391335), r | 5.51 (1.921-15.79) | 1.5 × 10⁻⁰³ | | | |
| EPHA3 (rs6419884), r | 0.03 (0.006-0.20) | 1.4 × 10⁻⁰⁴ | | | |
| EPHA4 (rs10498114), d | 0.20 (0.075-0.54) | 1.5 × 10⁻⁰³ | | | |
| EPHA4 (rs10498118), a | 0.24 (0.112-0.50) | 1.5 × 10⁻⁰⁴ | | | |
| EPHB1 (rs9871429), r | 10.15 (3.417-30.15) | 3.0 × 10⁻⁰⁵ | | | |
| EPHB1 (rs1404577), r | 0.04 (0.006-0.33) | 2.4 × 10⁻⁰³ | | | |
| EPHB1 (rs9865620), d | 0.14 (0.056-0.37) | 6.4 × 10⁻⁰⁵ | | | |
| EPHB1 (rs6793828), r | 0.06 (0.015-0.23) | 5.5 × 10⁻⁰⁵ | | | |
| FYN (rs809193), a | 34.03 (10.029-115.48) | 1.5 × 10⁻⁰⁸ | | | |
| GSK3B (rs3108749), a | 0.02 (0.002-0.10) | 9.8 × 10⁻⁰⁶ | | | |
| ITGB1 (rs11009157), a | 0.18 (0.084-0.37) | 4.3 × 10⁻⁰⁶ | | | |
| LIMK2 (rs5997921), a | 0.12 (0.046-0.30) | 9.3 × 10⁻⁰⁶ | | | |
| LRRC4C (rs1901583), a | 2.58 (1.374-4.83) | 3.2 × 10⁻⁰³ | | | |
| NGEF (rs4973578), r | 0.02 (0.004-0.11) | 2.5 × 10⁻⁰⁶ | | | |
| NTN1 (rs2091317), r | 0.09 (0.018-0.46) | 3.8 × 10⁻⁰³ | | | |
| NTN4 (rs4533108), d | 0.34 (0.149-0.79) | 1.2 × 10⁻⁰² | | | |
| NTNG1 (rs7411507), d | 8.18 (3.112-21.51) | 2.0 × 10⁻⁰⁵ | | | |
| PAK2 (rs9872035), r | 0.07 (0.019-0.23) | 2.2 × 10⁻⁰⁵ | | | |
| PAK7 (rs221015), d | 22.43 (7.218-69.67) | 7.5 × 10⁻⁰⁸ | | | |
| PAK7 (rs1033470), d | 2.89 (1.126-7.40) | 2.7 × 10⁻⁰² | | | |
| PLXNA2₁ (rs2782934), r | 0.03 (0.005-0.19) | 2.2 × 10⁻⁰⁴ | | | |
| PLXNA2₂ (rs473500), d | 2.62 (0.486-14.08) | 2.6 × 10⁻⁰¹ | | | |
| PLXNA2₃ (rs6685511), d | 13.34 (4.221-42.14) | 1.0 × 10⁻⁰⁵ | | | |
| PPP3CA (rs2732504), d | 0.25 (0.097-0.62) | 2.9 × 10⁻⁰³ | | | |
| PTK2 (rs4596627), d | 0.20 (0.076-0.53) | 1.2 × 10⁻⁰³ | | | |
| ROBO1 (rs723765), r | 0.10 (0.026-0.41) | 1.2 × 10⁻⁰³ | | | |
| ROBO1 (rs2255164), a | 3.87 (2.011-7.46) | 5.1 × 10⁻⁰⁵ | | | |
| ROBO2 (rs9852548), a | 0.26 (0.114-0.59) | 1.3 × 10⁻⁰³ | | | |
| ROBO2 (rs6779154), a | 4.23 (1.989-9.00) | 1.8 × 10⁻⁰⁴ | | | |
| ROCK1 (rs1481280), d | 3.09 (1.255-7.60) | 1.4 × 10⁻⁰² | | | |
| SEMA3A (rs7793598), d | 0.22 (0.080-0.60) | 3.3 × 10⁻⁰³ | | | |
| SEMA3A (rs10488266), a | 0.15 (0.046-0.46) | 1.0 × 10⁻⁰³ | | | |
| SEMA3C (rs3807094), a | 5.76 (2.269-14.64) | 2.3 × 10⁻⁰⁴ | | | |
| SEMA3E₁ (rs2722990), d | 0.09 (0.023-0.37) | 7.3 × 10⁻⁰⁴ | | | |
| SEMA3E₂ (rs2722964), d | 2.07 (0.811-5.30) | 1.3 × 10⁻⁰¹ | | | |
| SEMA4G (rs722435), d | 6.01 (2.334-15.47) | 2.0 × 10⁻⁰⁴ | | | |
| SEMA5A (rs2290735), a | 0.06 (0.014-0.23) | 4.9 × 10⁻⁰⁵ | | | |
| SEMA5B (rs2120806), d | 92.12 (18.175-466.87) | 4.7 × 10⁻⁰⁸ | | | |
| SEMA6A (rs187710), r | 25.02 (4.418-141.63) | 2.7 × 10⁻⁰⁴ | | | |
| SLIT1 (rs1336258), a | 0.07 (0.019-0.26) | 5.8 × 10⁻⁰⁵ | | | |
| SLIT2 (rs2196476), r | 19.53 (4.565-83.53) | 6.1 × 10⁻⁰⁵ | | | |
| SLIT3 (rs297884), d | 3.60 (1.439-8.99) | 6.2 × 10⁻⁰³ | | | |
| SLIT3 (rs884787), d | 28.40 (6.293-128.16) | 1.3 × 10⁻⁰⁵ | | | |
| SLIT3 (rs2304035), a | 2.68 (1.377-5.21) | 3.7 × 10⁻⁰³ | | | |
| SRGAP1 (rs10878106), d | 0.31 (0.126-0.78) | 1.2 × 10⁻⁰² | | | |
| SRGAP3 (rs610457), r | 0.01 (0.002-0.04) | 5.9 × 10⁻¹⁰ | | | |
| UNC5C (rs10516969), d | 5.29 (2.030-13.76) | 6.5 × 10⁻⁰⁴ | | | |
| UNC5C (rs265047), a | 10.10 (3.028-33.68) | 1.7 × 10⁻⁰⁴ | | | |
| UNC5C (rs4254782), r | 0.18 (0.056-0.55) | 2.9 × 10⁻⁰³ | | | |

*Subscript is used to identify individual SNPs in interactions.
**Results adjusted for age and gender.
^{†}a = log-additive, d = Mendelian dominant, r = Mendelian recessive.

### Survival Free of PD

Of the 3,095 SNPs, 327 (10.6%) were individually associated with survival free of PD (hazard function) using Cox proportional hazards models, as detailed in Table 16. Table 18 contains results for the final proportional hazards model produced by running SNPs through the multi-stage process to predict survival free of PD. This model used data from 264 PD patients (4 patients were missing data on one or more SNPs). In this case, the model had an overall p value of 9.02×10⁻⁵⁸ (95% CI 1.48×10⁻⁴⁶-7.90×10⁻⁷⁰). By contrast, the model was not significant at predicting survival (age at study) of the matched sibling controls (p=0.80). This last finding suggests that the model predicts survival free of PD (hazard function), but not survival in general, and that the model is specific for PD cases.

TABLE 18
SNPs in Axon Guidance Pathway Genes Predicting Survival Free of PD.

| Gene* (rs ID), | Hazard Ratio | | | Hazard Ratio | |
|---|---|---|---|---|---|
| Final Coding Scheme^{†} | (95% CI) | P value | Interaction* | (95% CI) | P value |
| CXCLI2 (rs266087), r | 2.36 (1.537-3.62) | 8.6 × 10⁻⁰⁵ | EPHA5₂* EPHB2 | 44.93 (6.386-316.06) | 1.3 × 10⁻⁰⁴ |
| DPYSL2 (rs2585459), r | 0.22 (0.131-0.36) | 2.5 × 10⁻⁰⁹ | NRP1* EPHB1₂ | 7.26 (3.008-17.52) | 1.0 × 10⁻⁰⁵ |
| EFNA5₁ (rs352602), r | 1.62 (0.864-3.02) | 1.3 × 10⁻⁰¹ | PAK7₁* NFATC2 | 12.33 (2.312-65.73) | 3.3 × 10⁻⁰³ |
| EPHA5₂ (rs13127631), r | 8.19 (3.446-19.45) | 1.9 × 10⁻⁰⁶ | EFNA5₁* EPHB1₂ | 16.84 (5.636-50.31) | 4.3 × 10⁻⁰⁷ |
| EPHA7 (rs164540), d | 2.18 (1.454-3.27) | 1.6 × 10⁻⁰⁴ | EPHA7* ROBO1 | 0.16 (0.044-0.56) | 4.3 × 10⁻⁰³ |
| EPHB1₁ (rs6439552), d | 0.63 (0.405-0.98) | 4.0 × 10⁻⁰² | EPHA5₂* EPHB1₂ | 0.03 (0.007-0.13) | 1.7 × 10⁻⁰⁶ |
| EPHB1₂ (rs11920780), d | 1.29 (0.907-1.85) | 1.5 × 10⁻⁰¹ | EPHA5₂* NTNG1₂ | 15.63 (4.400-55.53) | 2.1 × 10⁻⁰⁵ |
| EPHB2 (rs876685), a | 2.58 (1.854-3.59) | 1.9 × 10⁻⁰⁸ | PAK7₁* ROBO1 | 48.57 (6.627-355.98) | 1.3 × 10⁻⁰⁴ |
| ITGB1 (rs4587680), d | 0.32 (0.222-0.47) | 5.6 × 10⁻⁰⁹ | EPHB1₂* SRGAP3₂ | 0.10 (0.036-0.29) | 2.0 × 10⁻⁰⁵ |
| NFATC2 (rs4811184), r | 12.87 (2.592-63.93) | 1.8 × 10⁻⁰³ | ROCK1* SLIT1 | 3.14 (1.491-6.60) | 2.6 × 10⁻⁰³ |
| NGEF (rs1878289), r | 1.68 (1.119-2.51) | 1.2 × 10⁻⁰² | ITGB1* ROBO2 | 6.20 (1.478-26.02) | 1.3 × 10⁻⁰² |
| NRP1 (rs2776928), d | 1.43 (0.890-2.29) | 1.4 × 10⁻⁰¹ | EPHB1₁* EPHB2 | 0.26 (0.112-0.61) | 1.8 × 10⁻⁰³ |
| NTN1 (rs2302196), r | 0.20 (0.103-0.37) | 5.1 × 10⁻⁰⁷ | EPHA7* NTNG1₃ | 2.94 (1.419-6.09) | 3.7 × 10⁻⁰³ |
| NTN4 (rs4129599), r | 3.15 (1.366-7.24) | 7.1 × 10⁻⁰³ | PPP3CA * NTN1 | 2.22 (1.216-4.06) | 9.5 × 10⁻⁰³ |
| NTN4 (rs7137835), a | 1.56 (1.272-1.91) | 1.9 × 10⁻⁰⁵ | EPHA7* NFATC2 | 0.15 (0.027-0.83) | 3.0 × 10⁻⁰² |
| NTNG1₁ (rs7555385), a | 2.00 (1.617-2.47) | 1.6 × 10⁻¹⁰ | | | |
| NTNG1₂ (rs5012609), d | 0.04 (0.013-0.14) | 1.1 × 10⁻⁰⁷ | | | |
| NTNG1₃ (rs12146111), d | 6.69 (1.855-24.13) | 3.7 × 10⁻⁰³ | | | |
| PAK7₁ (rs7272361), d | 1.39 (0.830-2.33) | 2.1 × 10⁻⁰¹ | | | |
| PAK7₂ (rs3746545), r | 3.78 (2.068-6.90) | 1.5 × 10⁻⁰⁵ | | | |
| PLXNC1 (rs12579612), r | 8.49 (4.025-17.91) | 2.0 × 10⁻⁰⁸ | | | |
| PPP3CA (rs2732504), a | 0.48 (0.380-0.60) | 3.4 × 10⁻¹⁰ | | | |
| PPP3R2 (rs3739724), d | 2.22 (1.521-3.25) | 3.7 × 10⁻⁰⁵ | | | |
| ROBO1 (rs12486635), r | 9.60 (2.956-31.20) | 1.7 × 10⁻⁰⁴ | | | |
| ROBO2 (rs7640201), d | 7.39 (2.962-18.44) | 1.8 × 10⁻⁰⁵ | | | |
| ROCK1 (rs1481280), a | 1.34 (1.074-1.68) | 9.8 × 10⁻⁰³ | | | |
| SEMA5A (rs6873909), r | 6.53 (3.273-13.05) | 1.0 × 10⁻⁰⁷ | | | |
| SEMA6A (rs258014), d | 1.48 (1.092-2.00) | 1.1 × 10⁻⁰² | | | |
| SLIT1 (rs2279508), r | 0.14 (0.053-0.35) | 3.7 × 10⁻⁰⁵ | | | |
| SLIT2 (rs10516357), d | 1.58 (1.175-2.13) | 2.5 × 10⁻⁰³ | | | |
| SLIT3 (rs11954621), d | 0.48 (0.350-0.66) | 6.0 × 10⁻⁰⁶ | | | |
| SRGAP3₁ (rs1043951), a | 2.44 (1.721-3.47) | 6.0 × 10⁻⁰⁷ | | | |
| SRGAP3₂ (rs17530358), r | 8.05 (3.845-16.87) | 3.2 × 10⁻⁰⁸ | | | |
| UNC5C (rs7674643), r | 1.85 (1.228-2.78) | 3.2 × 10⁻⁰³ | | | |
| UNC5C (rs1483749), r | 2.28 (1.423-3.65) | 6.1 × 10⁻⁰⁴ | | | |

*Subscript is used to identify individual SNPs in interactions.
^{†}a = log-additive, d = Mendelian dominant, r = Mendelian recessive.

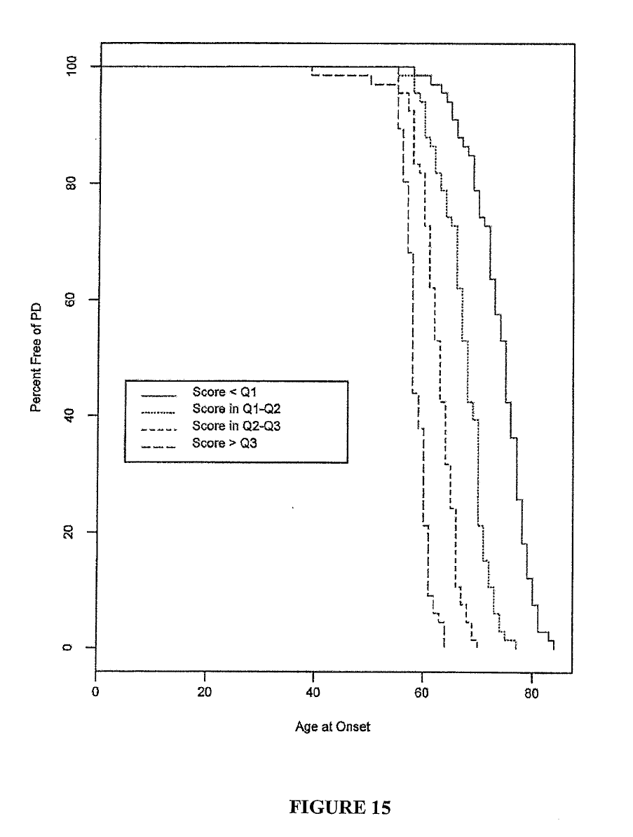

FIG. 15 shows a Kaplan-Meier plot to describe the results of the model. The groups were formed by calculating a risk score for each PD patient using the equation from the proportional hazards model, then categorizing the score at the 25^{th} (Q1), 50^{th} (Q2), and 75^{th} (Q3) percentiles. The survival curves separated nicely right from the earliest ages of onset. By age 60, only 21% of PD patients in the predicted highest risk group were still free of PD, whereas 98% of PD patients in the predicted lowest risk group were free of PD. By age 70, none of the PD patients in the predicted highest risk group were still free of PD, whereas 74% of PD patients in the predicted lowest risk group were free of PD. The median ages at onset for each group, from lowest risk group to highest risk group, were 75, 68, 63, and 58, a difference in survival free of PD of 17 years from lowest to highest. The concordance for this model was 0.85. The hazard ratios (95% CIs) for the four groups, from lowest to highest risk, were 1 (reference), 4.65 (3.05-7.08), 17.36 (10.54-28.57), and 72.90 (41.52-128.00).

### Age at Onset of PD

Of the 3,095 SNPs, 326 (10.5%) were individually associated with age at onset of PD using linear regression models, as detailed in Table 16. Table 19 contains results for the final model produced by running SNPs through the multi-stage process to predict PD age at onset. This model used data from 261 PD patients (7 patients were missing data on one or more SNPs). In this case, the model had an overall p value of 2.12×10⁻⁷¹ (95% CI 1.06×10⁻⁵²⁸-6.47×10⁻⁸¹). By contrast, the set of SNPs was not significant at predicting age at study of the matched sibling controls (p=0.98). This last finding suggests that the model predicts age at onset of PD, not age at the time of the study, and that the model is specific for PD cases.

TABLE 19
SNPs in Axon Guidance Pathway Genes Predicting Age at Onset of PD.

| | Regression | | | Regression | |
|---|---|---|---|---|---|
| Gene (rs ID), | coefficient | | | coefficient | |
| Final Coding Scheme^{†} | (standard error) | P value | Interaction* | (standard error) | P value |
| DCC₁ (rs1237775), a | −150.50 (51.53) | 3.9 × 10⁻⁰³ | DPYSL2* NTN1₂ | −928.33 (209.71) | 1.6 × 10⁻⁰⁵ |
| DCC₂ (rs6508153), d | 181.44 (58.96) | 2.4 × 10⁻⁰³ | EPHB1₁* NGEF | −654.77 (302.27) | 3.1 × 10⁻⁰² |
| DCC₃ (rs1221869), a | 229.56 (44.67) | 6.5 × 10⁻⁰⁷ | EPHA7* SLIT2 | −866.21 (175.30) | 1.6 × 10⁻⁰⁶ |
| DPYSL2 (rs2585459), r | 1104.14 (173.43) | 1.3 × 10⁻⁰⁹ | PTK2* DCC₃ | −333.36 (95.23) | 5.7 × 10⁻⁰⁴ |
| EFNA5₁ (rs252816), a | 142.05 (82.61) | 8.7 × 10⁻⁰² | SLIT2* PTK2 | −797.03 (192.86) | 5.2 × 10⁻⁰⁵ |
| EFNA5₂ (rs252991), d | 479.94 (60.88) | 1.9 × 10⁻¹³ | NTN4* PPP3CC | 551.80 (148.09) | 2.5 × 10⁻⁰⁴ |
| EPHA5₃ (rs13127631), r | −330.98 (119.84) | 6.3 × 10⁻⁰³ | EPHB1₃* SLIT2 | −1523.61 (486.87) | 2.0 × 10⁻⁰³ |
| EPHA7 (rs164540), d | −78.41 (68.15) | 2.5 × 10⁻⁰¹ | EPHB1₃* SRGAP3₁ | −1156.86 (306.03) | 2.1 × 10⁻⁰⁴ |
| EPHB1₁ (rs959562), d | 461.62 (86.58) | 2.6 × 10⁻⁰⁷ | EPHB2* SRGAP3₂ | 232.32 (88.20) | 9.1 × 10⁻⁰³ |
| EPHB1₂ (rs11920780), d | −290.82 (57.65) | 1.0 × 10⁻⁰⁶ | ROCK1* SEMA5B | −276.78 (101.91) | 7.2 × 10⁻⁰³ |
| EPHB1₃ (rs12488683), r | −180.50 (107.31) | 9.4 × 10⁻⁰² | ROCK1* SEMA4D | −359.40 (102.45) | 5.6 × 10⁻⁰⁴ |
| EPHB1₄ (rs2291989), r | −69.81 (85.34) | 4.1 × 10⁻⁰¹ | EFNA5₁* EPHB2 | −291.64 (94.46) | 2.3 × 10⁻⁰³ |
| EPHB2 (rs876685), a | −94.67 (103.33) | 3.6 × 10⁻⁰¹ | DCC₁* SEMA4D | −398.04 (124.96) | 1.7 × 10⁻⁰³ |
| ITGB1 (rs4587680), d | 257.62 (72.51) | 4.7 × 10⁻⁰⁴ | EPHB1₁* SLIT1 | −1440.10 (508.02) | 5.0 × 10⁻⁰³ |
| NGEF (rs2166441), r | 520.64 (126.72) | 5.8 × 10⁻⁰⁵ | EFNA5₁*SRGAP3₂ | −204.18 (64.16) | 1.7 × 10⁻⁰³ |
| NTN1₁ (rs1473664), r | 590.77 (121.55) | 2.3 × 10⁻⁰⁶ | SLIT3₂* EPHB1₄ | −418.63 (162.82) | 1.1 × 10⁻⁰² |
| NTN1₂ (rs4791331), d | −67.83 (71.23) | 3.4 × 10⁻⁰¹ | SLIT3₂* SRGAP3₂ | 247.52 (96.48) | 1.1 × 10⁻⁰² |
| NTN4 (rs11108230), a | 153.00 (69.47) | 2.9 × 10⁻⁰² | | | |
| NTNG1 (rs11803905), r | 363.75 (99.05) | 3.1 × 10⁻⁰⁴ | | | |
| PAK2 (rs11185455), a | 239.54 (49.45) | 2.5 × 10⁻⁰⁶ | | | |
| PPP3CC (rs2449340), r | 145.91 (81.37) | 7.4 × 10⁻⁰² | | | |
| PTK2 (rs4596627), d | 599.37 (110.56) | 1.7 × 10⁻⁰⁷ | | | |
| ROBO2 (rs7640201), d | −614.78 (132.96) | 6.7 × 10⁻⁰⁶ | | | |
| ROBO2 (rs1031377), r | 410.22 (81.18) | 9.7 × 10⁻⁰⁷ | | | |
| ROCK1 (rs1481280), a | −58.03 (52.79) | 2.7 × 10⁻⁰¹ | | | |
| ROCK2 (rs726843), r | 208.94 (65.01) | 1.5 × 10⁻⁰³ | | | |
| RRAS (rs1368464), d | 144.20 (55.57) | 1.0 × 10⁻⁰² | | | |
| SEMA3E (rs42002), r | −334.91 (80.40) | 4.6 × 10⁻⁰⁵ | | | |
| SEMA4D (rs9969727), r | 762.61 (119.60) | 1.2 × 10⁻⁰⁹ | | | |
| SEMA5A (rs40721), d | −229.78 (57.36) | 8.7 × 10⁻⁰⁵ | | | |
| SEMA5A (rs6873909), r | −531.17 (132.16) | 8.2 × 10⁻⁰⁵ | | | |
| SEMA5B (rs4677983), a | 605.55 (109.38) | 9.5 × 10⁻⁰⁸ | | | |
| SLIT1 (rs2279508), r | 286.19 (121.37) | 1.9 × 10⁻⁰² | | | |
| SLIT2 (rs636798), r | 1187.54 (148.70) | 1.0 × 10⁻¹³ | | | |
| SLIT3₁ (rs6864266), r | −351.13 (72.01) | 2.2 × 10⁻⁰⁶ | | | |
| SLIT3₂ (rs1432906), d | 138.69 (115.91) | 2.3 × 10⁻⁰¹ | | | |
| SLIT3₃ (rs11954621), d | 202.74 (60.83) | 1.0 × 10⁻⁰³ | | | |
| SRGAP3₁ (rs557377), r | 1204.44 (129.25) | 5.2 × 10⁻¹⁸ | | | |
| SRGAP3₂ (rs6777129), a | 242.30 (73.55) | 1.2 × 10⁻⁰³ | | | |
| UNC5C (rs7674643), r | −245.57 (76.16) | 1.5 × 10⁻⁰³ | | | |

*Subscript is used to identify individual SNPs in interactions.
^{†}a = log-additive, d = Mendelian dominant, r = Mendelian recessive.

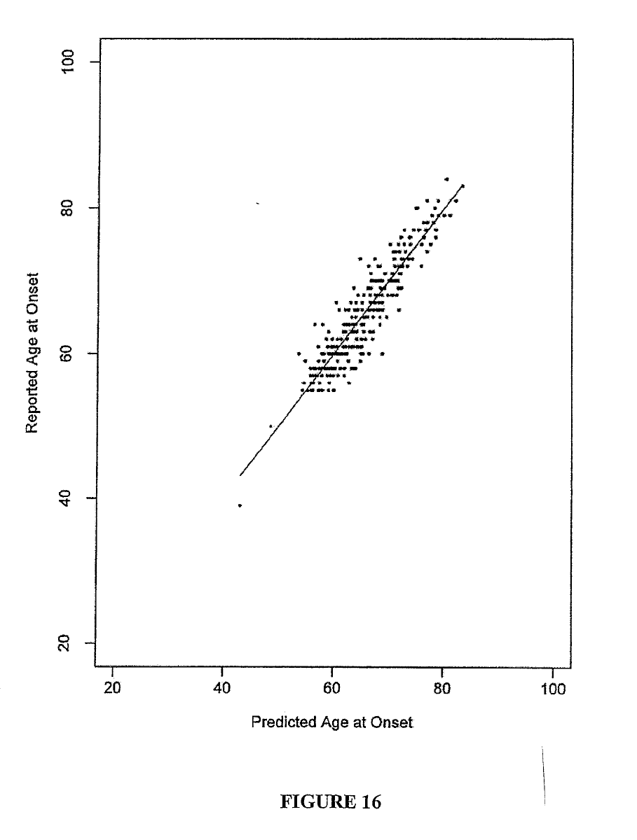

FIG. 16 shows a plot of predicted age at onset vs. reported age at onset to summarize the results of the model. The plot showed a nice elliptical pattern, reflecting the model R² of 0.86 (95% CI 0.83-0.89). The model explained about 86% of the variability in age at onset of PD.

Other combinations of SNPs from the axon guidance pathway also performed quite well in predicting PD susceptibility, survival free of PD, and age at onset of PD. Although the models provided herein provided good fits to the data, the results do not preclude other combinations of axon guidance pathway SNPs as significant predictors of PD. The SNPs in the final models selected showed no significant linkage disequilibrium in unrelated controls.

### Example 5

### Comparison of Final Models for ALS and PD from Examples 3 and 4

The final model predicting ALS susceptibility contained 31 genes, and the final model predicting PD susceptibility contained 39 genes. In total, the models contained 46 genes, of which 24 (52.2%) were shared, and 22 (47.8%) were not. Of the 22, 7 were in the ALS model but not the PD model, and 15 were in the PD model but not in the ALS model.

The final model predicting survival free of ALS contained 34 genes, and the final model predicting survival free of PD contained 28 genes. In total, the models contained 45 genes, of which 17 (37.8%) were shared, and 28 (62.2%) were not. Of the 28, 17 were in the ALS model but not the PD model, and 11 were in the PD model but not in the ALS model.

The final model predicting age at onset of ALS contained 30 genes, and the final model predicting age at onset of PD contained 28 genes. In total, the models contained 43 genes, of which 15 (34.9%) were shared, and 28 (65.1%) were not. Of the 28, 15 were in the ALS model but not the PD model, and 13 were in the PD model but not in the ALS model.

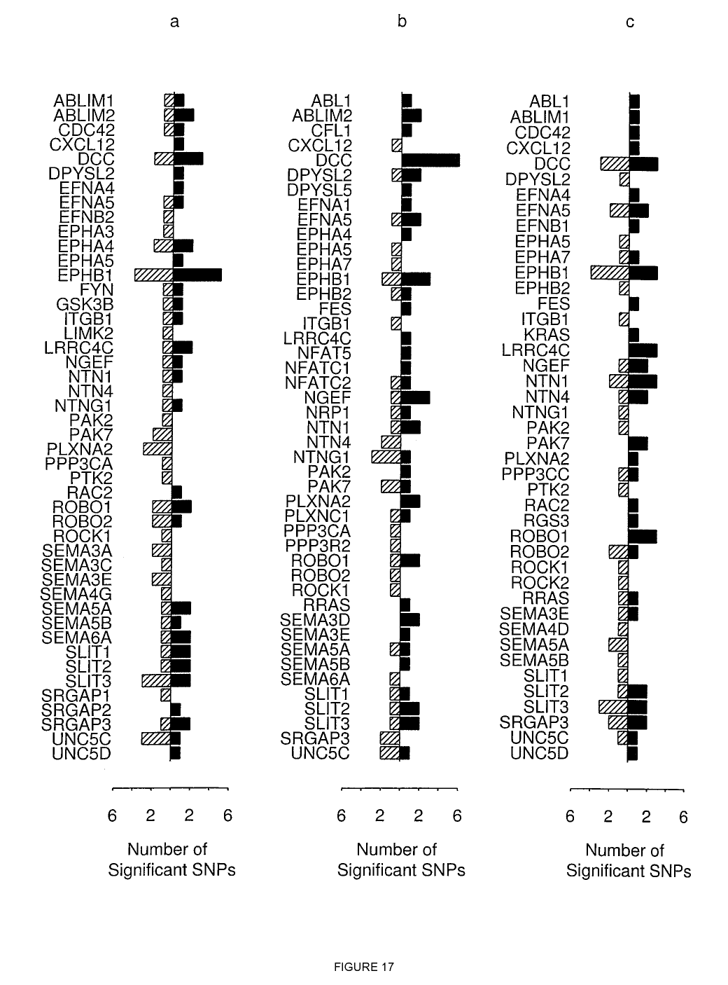

FIG. 17 displays the genes and numbers of significant SNPs in each. There are some interesting commonalities and differences. Since the ALS and PD data were generated using different chips, and the final models are not exclusive, other combinations of SNPs can have predictive value.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for assessing Parkinson's disease susceptibility, wherein said method comprises determining whether or not a human contains an axon guidance pathway genotype predisposing said human to develop Parkinson's disease.

2. The method of claim 1, wherein said determining step comprises sequencing nucleic acid from said human.

3. The method of claim 1, wherein said determining step comprises using a probe to determine the presence or absence of a polymorphism in said human.

4. The method of claim 1, wherein said genotype comprises at least two of the polymorphisms of Table 1.

5. A method for predicting age of Parkinson's disease onset in a human, wherein said method comprises:
(a) determining the presence or absence of polymorphisms in a set of axon guidance pathway genes of said human to obtain information about the axon guidance pathway genotype of said human, and
(b) calculating an age of Parkinson's disease onset for said human based on said information.

6. The method of claim 5, wherein said determining step comprises sequencing nucleic acid from said human.

7. The method of claim 5, wherein said determining step comprises using a probe to determine the presence or absence of a polymorphism in said human.

8. The method of claim 5, wherein said genotype comprises at least two of the polymorphisms of Table 3.

9. A method for diagnosing a brain disorder or assessing brain disorder susceptibility, wherein said method comprises using a model to determine whether or not a human contains an axon guidance pathway genotype diagnositic for said brain disorder or predisposing said human to develop said brain disorder, wherein said model comprises a model concordance of at least 0.6, an r-squared of at least 0.6, or a sensitivity and specificity of at least 0.6.

10. The method of claim 9, wherein said brain disorder is selected from the group consisting of Parkinson's disease, Alzheimer's disease, ALS, Tourette's syndrome, dyslexia, autism, mental retardation, epilepsy, stuttering, schizophrenia, addiction, anxiety, depression, obsessive-compulsive disorder, ADHD, MS, brain tumors, brain injury, and spinal cord injury.
